(19) United States
(12) Patent Application Publication
(10) Pub. No.: US 2009/0176975 A1
(43) Pub. Date: Jul. 9, 2009

(54) METHODS AND COMPOSITIONS FOR DETECTING PROMOTER ACTIVITY AND EXPRESSING FUSION PROTEINS

(75) Inventors: Harry Yim, Vista, CA (US); James Fan, Carlsbad, CA (US); Jonathan Chesnut, Carlsbad, CA (US); Kenneth Frimpong, San Diego, CA (US); Laura Vozza-Brown, Carlsbad, CA (US); Louis Leong, Junction City, OR (US); Peter Welch, San Diego, CA (US); Robert Bennett, Encinitas, CA (US)

Correspondence Address: INVITROGEN CORPORATION;C/O INTELLEVATE, P.O. BOX 52050, MINNEAPOLIS, MN, 55402, US

(73) Assignee: INVITROGEN CORPORATION, Carlsbad, CA (US)

(21) Appl. No.: 12/019,785

(22) Filed: Jan. 25, 2008

Related U.S. Application Data
(63) Continuation of application No. 10/877,952, filed on Jun. 28, 2004, now abandoned.
(60) Provisional application No. 60/511,634, filed on Oct. 17, 2003.
(60) Provisional application No. 60/487,301, filed on Jul. 16, 2003.
(60) Provisional application No. 60/482,504, filed on Jun. 26, 2003.

(51) Int. Cl. C07H 21/04

(52) U.S. Cl. 536/23.2

(57) ABSTRACT

The present invention provides nucleic acid molecules comprising one or more nucleic acid sequences encoding a polypeptide having a detectable activity. The present invention also provides methods of joining such nucleic acid molecules to nucleic acid molecules to be assayed for promoter activity. The present invention also relates to methods of preparing fusion proteins comprising a polypeptide of interest and a polypeptide having a detectable activity.

### CROSS-REFERENCE TO RELATED APPLICATIONS

This application claims the benefit of U.S. Provisional Patent Application Nos. 60/482,504, filed Jun. 26, 2003, 60/487,301, filed Jul. 16, 2003, and 60/511,634, filed Oct. 17, 2003, the contents of which are relied upon and incorporated by reference in their entireties.

### BACKGROUND OF THE INVENTION

1. Field of the Invention

The present invention relates to the fields of biotechnology and molecular biology. In particular, the present invention relates to the construction and use of nucleic acid molecules comprising sequences encoding polypeptides having a detectable activity. In a particular embodiment, the present invention relates to nucleic acid molecules encoding all or a portion of a polypeptide having β-lactamase activity.

2. Related Art

Reporter genes have found widespread use in the practice of biotechnology (see, for example, Molecular Cloning, second edition, editor J. Sambrook et al., Cold Spring Harbor Laboratory Press (1989)). One application of reporter genes is for the measurement of the promoter activity of a nucleotide sequence. This permits the identification of nucleotide sequences that promote the expression of particular sequences of interest in a host cell. This is particularly useful in identifying promoters that function in specific cell types (e.g., tissue-specific promoters).

To determine the promoter activity of a nucleic acid sequence, a nucleic acid molecule is constructed in which a nucleic acid sequence encoding a polypeptide having a detectable activity (i.e., a reporter gene) is operably linked to a nucleic acid sequence to be tested as a promoter. The nucleic acid molecule is then introduced in a host cell and the host cells are assayed for the presence and/or amount of the detectable activity. The amount of activity detected is indicative of the relative strength of the tested sequence as a promoter. Typically, reporter genes are selected for the ease with which their activity can be determined. Another consideration is whether the host cells contain an activity that can interfere with the assay.

Another use of reporter genes is in the construction of fusion proteins. Typically, a nucleic acid molecule is constructed such that a nucleic acid sequence encoding a polypeptide having a detectable activity (i.e., a reporter gene) is placed adjacent to a nucleic acid sequence encoding a polypeptide of interest. As is well known in the art, the two sequences may be placed such that the coding sequences of the two polypeptides are in the same reading frame. This results in the expression of a fusion polypeptide containing both the polypeptide encoded by the reporter gene and the polypeptide of interest. Cells containing the fusion polypeptide can be detected by assaying for the detectable activity.

Nucleic acid sequences encoding a wide variety of polypeptides have been used as reporter genes. Some of the polypeptides encoded include, but are not limited to, enzymes (e.g., chloramphenicol acetyl transferase, alkaline phosphatase, luciferase, β-galactosidase, β-glucuronidase, etc.) and fluorescent proteins (e.g., green fluorescent protein, yellow fluorescent protein, red fluorescent protein, cyan fluorescent protein, etc.). The β-lactamase gene has been used as a reporter and detection system for protein expression in mammalian cells (see, for example, Whitney et al. (1998) Nat. Biotechnol. 16:1329-33; and Zlokarnik, et al. (1998) Science 279:84-88).

After expression of a polypeptide in a host cell (e.g., a fusion polypeptide), it is typically necessary to separate the desired polypeptide from the other components of the host cell. Affinity chromatography is often the preferred method for polypeptide purification and can often be used to purify polypeptides from complex mixtures with high yield. Affinity chromatography is based on the ability of polypeptides to bind non-covalently but specifically to an immobilized ligand for the desired polypeptide. A number of peptides and polypeptides have been used for affinity chromatography, for example, the six histidine peptide, various epitopes (e.g., the V5 epitope), glutathione S-transferase (GST), the maltose-binding protein, etc. Peptides having an affinity for a biarsenical compound have been used for affinity purification (see, for example, U.S. Pat. Nos. 5,932,474, 6,008,378, 6,054,271, and 6,451,569 and published international patent application WO 01/53325A2).

### SUMMARY OF THE INVENTION

The present invention relates to nucleic acid sequences encoding polypeptides having a detectable activity and nucleic acid molecules comprising such sequences. Detectable activity may be any characteristic that can be detected, for example, enzymatic activity, fluorescence, binding activity, and the like. In some embodiments, detectable activity may be a β-lactamase activity. In particular embodiments, a detectable activity may be an activity that can alter the fluorescence (e.g., increase florescence yield, decrease fluorescence yield, change the emission wavelength, etc.) of a fluorescent substrate with which the polypeptide interacts. In some embodiments, a detectable activity may involve binding of the polypeptide to specific molecules (e.g., molecules comprising one or more arsenic atoms). Nucleic acid molecules of the invention may also comprise one or more (e.g., one, two, three, four, five, etc.) recombination sites (e.g., one or more att sites, one or more lox sites, etc.) and/or one or more (e.g., one, two, three, four, five, etc.) topoisomerase recognition sites (e.g., one or more recognition sites for a type IA topoisomerase, a type EB topoisomerase, a type II topoisomerase, etc.). Such nucleic acid molecules also include nucleic acid molecules that have undergone cleavage (e.g., cleavage of one strand of the nucleic acid molecules) with a topoisomerase (e.g., a site specific topoisomerase). Further, one or more topoisomerase molecules may be bound (e.g., covalently bound) to each nucleic acid molecule which is cleaved. Optionally, nucleic acid molecules comprising a sequence encoding a polypeptide having a detectable activity may comprise one or more recombination sites and/or one or more topoisomerases. The invention also relates to vectors comprising one or more nucleic acid molecules of the invention as well as variants and derivatives of these vectors.

In particular embodiments, the invention relates to combining or joining at least a first nucleic acid molecule which comprises at least a first nucleic acid sequence encoding a polypeptide having a detectable activity (e.g., a β-lactamase) and also comprises at least one topoisomerase site and/or topoisomerase and at least a second nucleic acid molecule that comprises a nucleic acid sequence to be assayed for promoter activity (e.g., a nucleic acid sequence which potentially has one or more activities associated with promoters). Optionally, the first nucleic acid molecule comprises one or more recombination sites. When a first nucleic acid molecule comprises two or more recombination sites, such sites may be engineered recombination sites and may not recombine or substantially recombine with each other. Optionally, a second nucleic acid molecule may comprise one or more topoisomerase recognition sites and/or one or more topoisomerases and/or one or more recombination sites. When a second nucleic acid molecule comprises two or more recombination sites, such sites may be engineered recombination sites and may not recombine with each other.

Upon joining the at least first and second molecules, the sequence encoding a polypeptide having a detectable activity may be operably-linked to the sequence to be assayed for promoter activity. These nucleic acid molecules may be linear or closed circular (e.g., relaxed, supercoiled, etc.). Such recombination sites, topoisomerase recognition sites and topoisomerases can be located at any position on any number of nucleic acid molecules of the invention, including at or near the termini of the nucleic acid molecules and/or within the nucleic acid molecules. Moreover, any combination of the same or different recombination sites, topoisomerase recognition sites and/or topoisomerases may be used in accordance with the invention.

The invention also relates to nucleic acid molecules comprising nucleic acid sequences encoding polypeptides having a detectable activity and also comprising one or more recombination sites. Optionally, such nucleic acid molecules may comprise two recombination sites that do not recombine with each other. Such recombination sites may be located anywhere in the nucleic acid molecule and may be located such that at least one of the recombination sites is adjacent to the sequence encoding a polypeptide having a detectable activity. Optionally, a recombination site may have a sequence that encodes one or more amino acids in one or more reading frames. In some embodiments, a recombination site having a sequence encoding one or more amino acids in one or more reading frames may be located adjacent to the sequence encoding a polypeptide having a detectable activity. In such embodiments, amino acids encoded by the recombination site may be in the same reading frame as the polypeptide having a detectable activity. Such embodiments may produce a fusion protein comprising the polypeptide having a detectable activity and a peptide having one or more amino acids encoded by the sequence of the recombination site. In some embodiments, the peptide having one or more amino acids encoded by the sequence of the recombination site may comprise all of the amino acids encoded by the recombination site.

In some aspects, the present invention provides one or more methods for making nucleic acid molecules. Such methods may entail: (a) providing a first nucleic acid molecule comprising a first nucleic acid sequence encoding a polypeptide having a detectable activity and at least a first recombination site; (b) providing a second nucleic acid molecule comprising a second nucleic acid sequence to be assayed as a promoter and at least a second recombination site; and (c) forming a mixture in vitro between said first and second nucleic acid molecules and at least one recombination protein, under conditions sufficient to cause recombination in vitro between said first and second recombination sites, thereby producing a third nucleic acid molecule in which said first and second nucleic acid sequences are operably linked. Methods of the invention may further comprise (d) contacting one or more hosts or host cells with said mixture; and (e) selecting for a host or host cell comprising said third nucleic acid molecule, and selecting against a host or host cell comprising said first nucleic acid molecule and against a host or host cell comprising said second nucleic acid molecule. In particular embodiments, the second nucleic acid molecule above may be a member of a population of nucleic acid molecules which differ in sequence. Thus, the invention include methods for identifying nucleic acid molecules present in a mixed population which have one or more activities of associated with a promoter.

In another aspect, methods of making nucleic acid molecules of the invention may entail: (a) providing a first nucleic acid molecule comprising a first nucleic acid sequence encoding a polypeptide having a detectable activity and at least a first recombination site; (b) providing a second nucleic acid molecule comprising a nucleic acid sequence encoding a polypeptide of interest and at least a second recombination site; and (c) forming a mixture in vitro between said first and second nucleic acid molecules and at least one recombination protein, under conditions sufficient to cause recombination in vitro between said first and second recombination sites, thereby producing a third nucleic acid molecule comprising a third nucleic acid sequence that encodes all or a portion of the polypeptide having a detectable activity and all or a portion of the polypeptide of interest in the same reading frame and comprising a third recombination site that is the product of the recombination of the first and second recombination sites. In methods of this type, in the third nucleic acid molecule, the third recombination site may be located between the nucleic acid sequence encoding a polypeptide having a detectable activity and the nucleic acid sequence encoding a polypeptide of interest. In some embodiments, a fusion protein comprising all or a portion of the amino acid sequence of the polypeptide having a detectable activity, all or a portion of the amino acid sequence of the polypeptide interest and comprising at least one amino acid encoded by the third recombination site may be produced from the third nucleic acid molecule.

The invention includes, in part, nucleic acid molecules and compositions comprising nucleic acid molecules (e.g., reaction mixtures), wherein the nucleic acid molecules comprise (1) at least one (e.g., one, two, three, four, five, six, seven eight, etc.) recombination site and (2) at least one (e.g., one, two, three, four, five, six, seven eight, etc.) topoisomerase (e.g., a covalently linked topoisomerase) or at least one (e.g., one, two, three, four, five, six, seven eight, etc.) topoisomerase recognition site. In particular embodiments, the topoisomerases or topoisomerase recognition sites, as well as the recombination sites, of the nucleic acid molecules referred to above can be either internal or at or near one or both termini. For example, one or more (e.g., one, two, three, four, five, six, seven eight, etc.) of the at least one topoisomerase or the at least one topoisomerase recognition site, as well as one or more of the at least one recombination site, can be located at or near a 5′ terminus, at or near a 3′ terminus, at or near both 5′ termini, at or near both 3′ termini, at or near a 5′ terminus and a 3′ terminus, at or near a 5′ terminus and both 3′ termini, or at or near a 3′ terminus and both 5′ termini. The invention further provides methods for preparing and using nucleic acid molecules and compositions of the invention.

In a specific aspect, the invention provides nucleic acid molecules which comprise at least a first nucleic acid sequence encoding a polypeptide having a detectable activity to which topoisomerases of various types (e.g., a type IA topoisomerase, a type IB topoisomerase, a type II topoisomerase, etc.) are attached (e.g., covalently bound). In another specific aspect, the invention provides nucleic acid molecules which comprise at least a first nucleic acid sequence encoding a polypeptide having a detectable activity which contains two or more topoisomerase recognition sites which are recognized by one or more types of topoisomerases. The present invention also provides methods for preparing and using compositions comprising such nucleic acid molecules. In many embodiments, these nucleic acid molecules will further comprise one or more (e.g., one, two, three, four, five, six, seven, eight, etc.) recombination sites.

The invention further provides methods for joining two or more nucleic acid segments, at least one of which comprises at least a first nucleic acid sequence encoding a polypeptide having a detectable activity, wherein at least one of the nucleic acid segments contains at least one topoisomerase or topoisomerase recognition site and/or one or more recombination sites. Further, when nucleic acid segments used in methods of the invention contain more than one (e.g., two, three, four, five, six, seven eight, etc.) topoisomerase, either on the same or different nucleic acid segments, these topoisomerases may be of the same type or of different types. Similarly, when nucleic acid segments used in methods of the invention contain more than one topoisomerase recognition site, either on the same or different nucleic acid segments, these topoisomerase recognition sites may be recognized by topoisomerases of the same type or of different types. Additionally, when nucleic acid segments used in methods of the invention contain one or more recombination sites, these recombination sites may be able to recombine with one or more recombination sites on the same or different nucleic acid segments. Thus, the invention provides methods for joining nucleic acid segments using methods employing any one topoisomerase or topoisomerase recognition site. The invention provides further methods for joining nucleic acid segments using methods employing (1) any combination of topoisomerases or topoisomerase recognition sites and/or (2) any combination of recombination sites. The invention also provides nucleic acid molecules produced by the methods described above, as well as uses of these molecules and compositions comprising these molecules.

In general, the invention provides, in part, methods for joining one or more nucleic acid molecules or segments which comprises at least a first nucleic acid sequence encoding a polypeptide having a detectable activity with any number of nucleic acid segments (e.g., two, three, four, five, six, seven, eight, nine, ten, etc.) which contain different functional or structural elements. The invention thus provides, in part, methods for bringing together any number of nucleic acid segments (e.g., two, three, four, five, six, seven, eight, nine, ten, etc.) which confer different properties upon a nucleic acid molecule product. In many instances, methods of the invention will result in the formation of nucleic acid molecules wherein there is operable interaction between properties and/or elements of individual nucleic acid segments which are joined (e.g., operable interaction/linkage between an expression control sequence and at least a first nucleic acid sequence encoding a polypeptide having a detectable activity). Examples of (1) functional and structural elements and (2) properties which may be conferred upon product molecules include, but are not limited to, multiple cloning sites (e.g., nucleic acid regions which contain at least two restriction endonuclease cleavage sites), packaging signals (e.g., adenoviral packaging signals, alphaviral packaging signals, etc.), restriction endonuclease cleavage sites, open reading frames (e.g., intein coding sequence, affinity purification tag coding sequences, etc.), expression control sequences (e.g., promoters, operators, etc.), etc. Additional elements and properties which can be conferred by nucleic acid segments upon a product nucleic acid molecule are described elsewhere herein. The invention also provides nucleic acid molecules produced by the methods described above, as well as uses of these molecules and compositions comprising these molecules.

The invention further includes, in part, methods for joining two or more (e.g., 2, 3, 4, 5, 6, 7, 8, etc.) nucleic acid segments, wherein at least one (e.g., 1, 2, 3, 4, 5, 6, 7, 8, etc.) of the nucleic acid segments comprises at least a first nucleic acid sequence encoding a polypeptide having a detectable activity and comprises one or more (e.g., 1, 2, 3, 4, 5, 6, 7, 8, etc.) topoisomerases and/or one or more topoisomerase recognition sites and comprises one or more recombination sites. Thus, methods of the invention can be used to prepare joined or chimeric nucleic acid molecules by the joining of nucleic acid segments, wherein the product nucleic acid molecules comprise (1) one or more (e.g., 1, 2, 3, 4, 5, 6, 7, 8, etc.) topoisomerases and/or one or more (e.g., 1, 2, 3, 4, 5, 6, 7, 8, etc.) topoisomerase recognition sites and (2) one or more (e.g., 1, 2, 3, 4, 5, 6, 7, 8, etc.) recombination sites. The invention further provides nucleic acid molecules prepared by such methods, compositions comprising such nucleic acid molecules, and methods for using such nucleic acid molecules.

The invention also provides compositions comprising one or more nucleic acid segments and/or nucleic acid molecules described herein. Such compositions may comprise one or a number of other components selected from the group consisting of one or more other nucleic acid molecules (which may comprise recombination sites, topoisomerase recognition sites, topoisomerases, etc.), one or more nucleotides, one or more polymerases, one or more reverse transcriptases, one or more recombination proteins, one or more topoisomerases, one or more buffers and/or salts, one or more solid supports, one or more polyamines, one or more vectors, one or more restriction enzymes and the like. For example, compositions of the invention include, but are not limited to, mixtures (e.g., reaction mixtures) comprising a nucleic acid segment comprising a first nucleic acid sequence encoding a polypeptide having a detectable activity and at least one topoisomerase recognition site, and at least one topoisomerase which recognizes at least one of the at least one topoisomerase recognition sites of the nucleic acid segment. Compositions of the invention further include at least one nucleic acid segment comprising (1) a first nucleic acid sequence encoding a polypeptide having a detectable activity and at least one topoisomerase recognition site or at least one nucleic acid segment comprising a first nucleic acid sequence encoding a polypeptide having a detectable activity to which at least one topoisomerase is attached (e.g., covalently bound) and (2) one or more additional components. Examples of such additional components include, but are not limited to, topoisomerases; additional nucleic acid segments, which may or may not comprise one or more topoisomerases or topoisomerase recognition sites; buffers; salts; polyamines (e.g., spermine, spermidine, etc.); water; etc. Nucleic acid segments present in compositions of the invention may further comprise one or more recombination sites and/or one or more recombinase.

Often, nucleic acid molecules which have undergone cleavage with a topoisomerase (e.g., a site specific topoisomerase) will further have a topoisomerase molecule covalently bound to a phosphate group of the nucleic acid molecules. The invention further includes methods for preparing nucleic acid molecules described above and elsewhere herein, as well as recombinant methods for using such molecules.

In particular embodiments, nucleic acid molecules of the invention will be vectors. In additional embodiments, the invention includes host cells which contain nucleic acid molecules of the invention, as well as methods for making and using such host cells, for example, to produce expression products (e.g., proteins, polypeptides, antigens, antigenic determinants, epitopes, and the like, or fragments thereof).

In specific embodiments, nucleic acid molecules of the invention comprise two or more recombination sites with one or more (e.g., one, two, three, four, five, etc.) topoisomerase recognition site located between the recombination sites and comprise a first nucleic acid sequence encoding a polypeptide having a detectable activity that may be located outside the recombination sites.

In additional specific embodiments, circular nucleic acid molecules of the invention comprise two recombination sites with two topoisomerase recognition sites located between the two recombination sites and comprise a first nucleic acid sequence encoding a polypeptide having a detectable activity that may be located outside the recombination sites. Thus, if such molecules are linearized by cleavage between the topoisomerase recognition sites, the topoisomerase recognition sites in the resulting linear molecule will be located distal (i.e., closer to the two ends of the linear molecule) to the recombination sites and the sequence encoding a polypeptide having a detectable activity will be between the recombination sites. The invention thus provides linear nucleic acid molecules which contain at least a first nucleic acid sequence encoding a polypeptide having a detectable activity and one or more recombination sites and one or more topoisomerase recognition sites. In particular embodiments, the one or more topoisomerase recognition sites are located distal to the one or more recombination sites.

Recombination sites for use in the invention may be any recognition sequence on a nucleic acid molecule which participates in a recombination reaction catalyzed or facilitated by recombination proteins. In those embodiments of the present invention utilizing more than one recombination site, such recombination sites may be the same or different and may recombine with each other or may not recombine or not substantially recombine with each other. Recombination sites contemplated by the invention also include mutants, derivatives or variants of wild-type or naturally occurring recombination sites. Recombination site modifications include those that enhance recombination, such enhancement selected from the group consisting of substantially (i) favoring integrative recombination; (ii) favoring excisive recombination; (iii) relieving the requirement for host factors; (iv) increasing the efficiency of co-integrate or product formation; and (v) increasing the specificity of co-integrate or product formation. Particular modifications include those that enhance recombination specificity, remove one or more stop codons, and/or avoid hair-pin formation. Desired modifications can also be made to the recombination sites to include desired amino acid changes to the transcription or translation product (e.g., mRNA or protein) when translation or transcription occurs across the modified recombination site. Recombination sites that may be used in accordance with the invention include att sites, frt sites, dif sites, psi sites, cer sites, and lox sites or mutants, derivatives and variants thereof (or combinations thereof). Recombination sites contemplated by the invention also include portions of such recombination sites.

Topoisomerase recognition sites advantageously used in the nucleic acid molecules of this aspect of the invention will often be recognized and bound by a type I topoisomerase (such as type IA topoisomerases (including but not limited to E. coli topoisomerase I, E. coli topoisomerase III, eukaryotic topoisomerase II, archeal reverse gyrase, yeast topoisomerase III, Drosophila topoisomerase III, human topoisomerase III, Streptococcus pneumoniae topoisomerase III, and the traE protein of plasmid RP4) and type IB topoisomerases (including but not limited to eukaryotic nuclear type I topoisomerase and a poxvirus (such as that isolated from or produced by vaccinia virus, Shope fibroma virus, ORF virus, fowlpox virus, molluscum contagiosum virus and Amsacta moorei entomopoxvirus)), and type II topoisomerase (including, but not limited to, bacterial gyrase, bacterial DNA topoisomerase IV, eukaryotic DNA topoisomerase II (such as calf thymus type II topoisomerase), and T-even phage-encoded DNA topoisomerase).

Each starting nucleic acid molecule may comprise, in addition to at least a first nucleic acid sequence encoding a polypeptide having a detectable activity, a variety of sequences (or combinations thereof) including, but not limited to one or more recombination sites and/or one or more topoisomerase recognition sites and/or one or more topoisomerases, sequences suitable for use as primer sites (e.g., sequences which a primer such as a sequencing primer or amplification primer may hybridize to initiate nucleic acid synthesis, amplification or sequencing), transcription or translation signals or regulatory sequences such as promoters and/or operators, ribosomal binding sites, Kozak sequences, and start codons, transcription and/or translation termination signals such as stop codons (which may be optimally suppressed by one or more suppressor tRNA molecules), tRNAs (e.g., suppressor tRNAs), origins of replication, selectable markers, and genes or portions of genes which may be used to create protein fusion (e.g., N-terminal or carboxy terminal) such as GST, GUS, GFP, open reading frame (orf) sequences, and any other sequence of interest which may be desired or used in various molecular biology techniques including sequences for use in homologous recombination (e.g., gene targeting).

In another aspect of the invention, nucleic acid molecules of the invention include those which contain at least (1) one or more (e.g., one, two, three, four, five, six, seven, eight, nine, etc.) components of one or more of the vectors represented in FIGS. 1, 7, 8, 9, 13, 14, 15, 16, 17, 18, 19, 26, 30, 31, 32, 33, 34, 35, 36, 37, 41, 43, 44, 45, 46, 52 and/or 53, or (2) one or more components of such vectors which confer the same or similar feature upon a nucleic acid molecule. As a specific example, a nucleic acid molecule of the invention may be a vector which comprises, in addition to recombination sites, at least one blasticidin resistance marker (see, e.g., FIG. 30), at least one CMV promoter (see, e.g., FIG. 30), at least one EM7 promoter (see, e.g., FIG. 37A), at least one ampicillin resistance marker (see, e.g., FIG. 37A), and at least one bacterial origin of replication (see, e.g., FIG. 37A). In most instances, the combinations of components selected for inclusion in a nucleic acid molecule will be designed to provide activities intended for a particular use. For example, a vector which is capable of expressing a nucleic acid insert in more than one type of eukaryotic cells (e.g., human cells and insect cells) and is replicable in prokaryotic cells (e.g., E. coli cells) may be desired. Thus, the components which are selected for inclusion in nucleic acid molecules of the invention will typically be determined by the particular use for which it is designed. The invention further includes methods for making and using such nucleic acid molecules as described, for example, elsewhere herein.

In one embodiment, a method of the invention is performed such that the first nucleic acid molecule (which may be ss or ds), as well as other nucleic acids used in methods of the invention, comprises at least a first nucleic acid sequence encoding a polypeptide having a detectable activity, and a second nucleic acid molecule (which may be ss or ds) is one of a plurality of nucleotide sequences, for example, a library, a combinatorial library of nucleotide sequences, or a variegated population of nucleotide sequences.

The present invention also relates to compositions prepared according to the methods of the invention, and to compositions useful for practicing the methods. Such compositions can include one or more reactants used in the methods of the invention and/or one or more ds recombinant nucleic acid molecules produced according to a method of the invention. Such compositions can include, for example, one or more nucleic acid molecules having at least one nucleic acid sequence encoding a polypeptide having a detectable activity, one or more nucleic acid molecules with one or more topoisomerase recognition sites; one or more topoisomerase-charge nucleic acid molecules; one or more nucleic acid molecules comprising one or more recombination sites; one or more primers useful for preparing a nucleic acid molecule containing a topoisomerase recognition site at one or both termini of one or both ends of an amplification product prepared using the primer; one or more topoisomerases; one or more substrate nucleic acid molecules, including, for example, nucleotide sequences encoding tags, markers, regulatory elements, or the like; one or more covalently linked ds recombinant nucleic acid molecules produced according to a method of the invention; one or more cells containing or useful for containing a nucleic acid molecule, primer, or recombinant nucleic acid molecule as disclosed herein; one or more polymerases for performing a primer extension or amplification reaction; one or more reaction buffers; and the like. In one embodiment, a composition of the invention comprises two or more different topoisomerase-charged nucleic acid molecules and/or two or more different recombination sites. The composition can further comprise at least one topoisomerase. A composition of the invention also can comprise a site specific topoisomerase and a covalently linked ds recombinant nucleic acid molecule, wherein the recombinant nucleic acid molecule contains at least one topoisomerase recognition site for the site specific topoisomerase in each strand, and wherein a topoisomerase recognition site in one strand is within about 100 nucleotides of a topoisomerase recognition site in the complementary strand, generally within about five, ten, twenty or thirty nucleotides.

Methods of the invention may comprise expressing a protein from one or more nucleic acid molecules of the invention. Protein expression steps, according to the invention, may comprise:

(a) obtaining a nucleic acid molecule to be expressed which comprises one or more expression signals; and

(b) expressing all or a portion of the nucleic acid molecule under control of said expression signal thereby producing a peptide or protein encoded by said molecule or portion thereof.

In this context, the expression signal may be said to be operably linked to the sequence to be expressed. The protein or peptide expressed is will often be expressed in a host cell (in vivo), although expression may be conducted in vitro using techniques well known in the art. Upon expression of the protein or peptide, the protein or peptide product may optionally be isolated or purified.

Compositions, methods and kits of the invention may be prepared and carried out using a phage-lambda site-specific recombination system. Further, such compositions, methods and kits may be prepared and carried out using the GATEWAY® Recombinational Cloning System and/or the TOPO® Cloning System and/or the pENTR Directional TOPO® Cloning System, which are available from Invitrogen Corporation (Carlsbad, Calif.).

Recombination sites and topoisomerase recognition sites used in the methods of this aspect of the invention include, but are not limited to, those described elsewhere herein. In particular methods, nucleic acid molecules of the invention are joined with other nucleic acid molecules in the presence of at least one recombination protein, which may be but is not limited to Cre, Int, IHF, Xis, Fis, Hin, Gin, Cin, Tn3 resolvase, TndX, XerC, or XerD. In certain such embodiments, the recombination protein is Cre, Int, Xis, IHF or Fis.

The invention also provides kits comprising these isolated nucleic acid molecules of the invention, which may optionally comprise one or more additional components selected from the group consisting of one or more topoisomerases, one or more recombination proteins, one or more vectors, one or more polypeptides having polymerase activity, and one or more host cells.

Other embodiments of the invention will be apparent to one or ordinary skill in the art in light of what is known in the art, in light of the following drawings and description of the invention, and in light of the claims.

### BRIEF DESCRIPTION OF THE DRAWINGS/FIGURES

FIG. 1 is a schematic representation of a basic recombinational cloning reaction.

FIG. 2 provides the structure of the fluorescent substrate CCF2-AM.

FIG. 3 provides the structure of the fluorescent substrate CCF4-AM.

FIG. 4 provides a schematic representation of the hydrolysis of the fluorescent substrates used in some embodiments of the invention.

FIGS. 5A-5D illustrate various embodiments of compositions and methods of the invention for generating a covalently linked ds recombinant nucleic acid molecule. Topoisomerase is shown as a solid circle, and is either attached to a terminus of a substrate nucleic acid molecule or is released following a linking reaction. As illustrated, the substrate nucleic acid molecules have 5′ overhangs, although they similarly can have 3′ overhangs or can be blunt ended. In addition, while the illustrated nucleic acid molecules are shown having the topoisomerases bound thereto (topoisomerase-charged), one or more of the termini shown as having a topoisomerase bound thereto also can be represented as having a topoisomerase recognition site, in which case the joining reaction would further require addition of one or more site specific topoisomerases, as appropriate.

FIG. 5A shows a first nucleic acid molecule having a topoisomerase linked to each of the 5′ terminus and 3′ terminus of one end, and further shows linkage of the first nucleic acid molecule to a second nucleic acid molecule.

FIG. 5B shows a first nucleic acid molecule having a topoisomerase bound to the 3′ terminus of one end, and a second nucleic acid molecule having a topoisomerase bound to the 3′ terminus of one end, and further shows a covalently linked ds recombinant nucleic acid molecule generated due to contacting the ends containing the topoisomerase-charged substrate nucleic acid molecules.

FIG. 5C shows a first nucleic acid molecule having a topoisomerase bound to the 5′ terminus of one end, and a second nucleic acid molecule having a topoisomerase bound to the 5′ terminus of one end, and further shows a covalently linked ds recombinant nucleic acid molecule generated due to contacting the ends containing the topoisomerase-charged substrate nucleic acid molecules.

FIG. 5D shows a nucleic acid molecule having a topoisomerase linked to each of the 5′ terminus and 3′ terminus of both ends, and further shows linkage of the topoisomerase-charged nucleic acid molecule to two nucleic acid molecules, one at each end. The topoisomerases at each of the 5′ termini and/or at each of the 3′ termini can be the same or different.

FIG. 6 provides a schematic representation of directionally controlled topoisomerase mediated joining of nucleic acid molecules.

FIG. 7 provides a vector map of pGeneBLAzer™-TOPO®.

FIG. 8 is a vector map of pGeneBLAzer™/UBC.

FIG. 9 provides the nucleotide sequence of the TOPO® cloning site of pGeneBLAzer-TOPO® (SEQ ID NO: 1). The partial amino acid sequence of the β-lactamase reporter is also shown (SEQ ID NO:2).

FIGS. 10A-10B show a schematic representation of how FRET in the fluorescent substrate CCF2 is abolished upon hydrolysis by a β-lactamase (FIG. 10A) and a graph showing the change in fluorescence wavelength upon hydrolysis of CCF2 (FIG. 10B).

FIGS. 11A-11B provide the structure of CCF2-FA (FIG. 11A) and the structure of CCF2-AM (FIG. 11B).

FIG. 12 is a schematic representation showing the conversion of CCF2-AM into CCF2-FA upon uptake by a cell.

FIG. 13 provides a vector map of pcDNA™6.2/cGeneBLAzer™-DEST.

FIG. 14 provides a vector map of pcDNA™6.2/nGeneBLAzer™-DEST.

FIG. 15 provides the nucleotide sequence of the recombination region of pcDNA™6.2/cGeneBLAzer™-DEST (SEQ ID NO:3). The amino acid sequence encoded by a portion of this region is also shown. (SEQ ID NO:4).

FIG. 16 provides the nucleotide sequence of the recombination region of pcDNA™6.2/nGeneBLAzer™-DEST (SEQ ID NO:5). The amino acid sequence encoded by a portion of this region is also shown. (SEQ ID NO:6).

FIG. 17 provides a vector map of pcDNA™6.2/cGeneBLAzer™-GW/lacZ.

FIG. 18 provides a vector map of pcDNA™6.2/nGeneBLAzer™-GW/lacZ.

FIG. 19 provides a vector map of supercoiled pGeneBLAzer™.

FIG. 20 shows photographs of cells transfected with various pGeneBLAzer™ constructs and loaded with a fluorescent β-lactamase substrate.

FIGS. 21A and 21B provide graphs of β-lactamase activity in cells transfected with various pGeneBLAzer™ constructs.

FIGS. 22A-22D show photographs of cells transfected with pcDNA™6.2/cGeneBLAzer™-GW/lacZ and pcDNA™6.2/nGeneBLAzer™-GW/lacZ constructs and loaded with a fluorescent β-lactamase substrate.

FIGS. 23A-23B show the analysis of cells transfected with pcDNA™6.2/cGeneBLAzer™-GW/lacZ and pcDNA™6.2/nGeneBLAzer™-GW/lacZ constructs by Western blot (23A) and Tropix assay (23B).

FIGS. 24A-24E shows a comparison of photographs of cells transfected with either pcDNA™6.2/FRTNV5-2-GW/GeneBLAzer™, pcDNA™6.2/nGeneBLAzer™-GW/lacZ, or pcDNA™6.2/cGeneBLAzer™-GW/lacZ and loaded with the fluorescent β-lactamase substrate CCF4-AM.

FIGS. 25A-25B show a comparison of activity measured in cells transfected with either pcDNA™6.2/FRTNV572-GW/GeneBLAzer™, pcDNA™6.2/nGeneBLAzer™-GW/lacZ, or pcDNA™6.2/cGeneBLAzer™-GW/lacZ and loaded with the fluorescent β-lactamase substrate CCF4-AM.

FIG. 26 provide a vector map of pENTR Spec-ccdB D-Topo, which contains a spectinomycin resistance marker (labeled “aad A”).

FIGS. 27A-27B shows the results of an assay of 293 cells transfected with various constructs.

FIGS. 28A-28C show photographs of cells transfected with various constructs and loaded with fluorescent substrate.

FIG. 29 shows a Western blot of cells transfected with various constructs.

FIG. 30 provides a vector map of pcDNA™6.2/cFLASH™-DEST an exemplary vector of the invention.

FIG. 31 provides a vector map of pcDNA™6.2/nFLASH™-DEST an exemplary vector of the invention.

FIG. 32 provides a vector map of pcDNA™6.2/cFLASH™ GW/TOPO an exemplary vector of the invention.

FIG. 33 provides a vector map pcDNA™6.2/nFLASH™ GW/TOPO an exemplary vector of the invention.

FIG. 34 provides a vector map of pcDNA™6.2/cGeneBLAzer™GW/DTOPO an exemplary vector of the invention.

FIG. 35 provides a vector map of pcDNA™6.2/nGeneBLAzer™ GW/DTOPO an exemplary vector of the invention.

FIG. 36 provides a vector map of plasmid D-T Entry ccdb spec an exemplary vector of the invention.

FIGS. 37A-37B provide a vector map of pcDNA™6.2/cGeneBLAzer™ GW/D.3 an exemplary vector of the invention (FIG. 37A) and a vector map of pcDNA™6.2/nGeneBLAzer™ GW/D.3 (FIG. 37B), which are exemplary vectors of the invention.

FIGS. 38A-38B provide the chemical structure of FLASH™-EDT₂ (FIG. 38A) and the chemical structure of REASH-EDT₂ (FIG. 38B), which are examples of a molecule containing one or more arsenic atoms according to specific aspects of the invention.

FIG. 39 provides the sequences of oligonucleotides useful for TOPO-adapting nucleic acid molecules of the invention: D92 (SEQ ID NO:7), D91 (SEQ ID NO:8), D90 (SEQ ID NO:9), D89 (SEQ ID NO:10), D76 (SEQ ID NO:11), D75 (SEQ ID NO:12), D74 (SEQ ID NO:13), D73 (SEQ ID NO:14), D72 (SEQ ID NO:15), D71 (SEQ ID NO:16), and D70 (SEQ ID NO:17).

FIG. 40 provides the amino acid sequence of a polypeptide having β-lactamase activity (SEQ ID NO:18).

FIG. 41 provides a vector map of pENTR/GeneBLAzer™, a nucleic acid molecule of the invention.

FIG. 42 shows a tetracysteine motif and the binding of this motif to form a chemical complex. LUMIO™ is a labeling technology that relies upon covalent bond formation between organo-arsenicals and pairs of thiols. This schematic representation depicts the formation of the fluorescent complex when the FLASH™ reagent binds to the tetracysteine motif in the target protein.

FIGS. 43A-43H show a map of pET160-DEST (also referred to as pET160/LUMIO™-DEST and pET160/Smartag-DEST) and annotated sequence data (SEQ ID NO: 19). The amino acid sequence encoded by a portion of this sequence is also shown. (SEQ ID NO:20). Features of the pET160 vectors include an N-terminal His, a LUMIO™ tag and TEV protease recognition site.

FIGS. 44A-44I show a map of pET161-DEST (also referred to as pET161/LUMIO™-DEST and pET161/Smartag-DEST) and annotated sequence data (SEQ ID NO:21). The amino acid sequences encoded by portions of this sequence are also shown. (sequence before attR1: SEQ ID NO:22, sequence encoded by chloramphenicol resistance gene: SEQ ID NO:23, sequence encoded by ccdb: SEQ ID NO:24, sequence encoded by Smartag+6His SEQ ID NO:25, sequence encoded by AP(R): SEQ ID NO:26, sequence of Rop protein: SEQ ID NO:27, sequence encoded by lacI: SEQ ID NO:28). Features of the pET161 include an N-terminal RBS, start codon and translational enhancer along with a C-terminal LUMIO™ tag-His epitope.

FIGS. 45A-45H show a map of pET160/D-TOPO™ and annotated sequence data (SEQ ID NO:29). The amino acid sequences encoded by portions of this sequence are also shown. (His6+FLASH sequence: SEQ ID NO:30, ROP sequence: SEQ ID NO:31, lacI sequence: SEQ ID NO:32). Features of the pET160 include an N-terminal His, a LUMIO™ tag and TEV protease recognition site.

FIGS. 46A-46H show a map of pET161/D-TOPO™ and annotated sequence data (SEQ ID NO:33). The amino acid sequences encoded by portions of this sequence are also shown. (Sequence before DTopo Site: SEQ ID NO:34, Smartag+6His sequence: SEQ ID NO:35, AP(R) sequence: SEQ ID NO:36, Rop protein sequence: SEQ ID NO:37, lacI sequence: SEQ ID NO:38). Features of the pET161 include an N-terminal RBS, start codon and translational enhancer along with a C-terminal LUMIO™ tag-His epitope.

FIGS. 47A-47D show the detection of proteins expressed from pET160 control vectors. Cell lysates from expression of pET160-GW-CAT and pET160/DT-CAT were analyzed by SIMPLYBLUE™ staining (panel A), In-Gel detection of LUMIO™ tag (panel B), and Western blotting (panel C) with the mouse anti-HisG antibody and the WESTERNBREEZE® Chemiluminescent Detection Kit (Anti-Mouse) (Invitrogen Corp., Carlsbad, Calif., cat. no. WB7104). Tagged protein was also detected by UV illumination of the PVDF filter after protein transfer (panel D). Lanes 1 and 3 are uninduced samples, lane 2 is induced pET160-GW-CAT and lane 4 is induced pET160/DT-CAT. Lane 5 of panel A and B are SEEBLUE® Standards, and lane 5 of panel C is the MAGICMARK™ Marker.

FIGS. 48A-48B show IMAC purification of proteins expressed from pET160 and pET161 vectors. Panel A shows IMAC purification or proteins expressed using pET160-GW-CAT. The purification profile is shown with the sample lystate (Lane 1), flow-through (lane 2), six washes (lanes 3 thru 8), the elution fractions (lanes 9 thru 13) and SEEBLUE® markers (Lanes 15). Panel B shows IMAC purification or proteins expressed using pET161-kinase H5. The purification profile is shown with the sample lysate (lane 1), the flow-through (lane 2) and several washes (lanes 3-5). The elution fractions (lanes 6-10) and SEEBLUE® markers (lane 11).

FIGS. 49A-49H show in-gel detection of LUMIO™ labeled human kinase 96-well plate expression. The lysates from the 96-well Human Kinase Plate were run on 4-20% Tris-Glycine gels and observed on a UV light box.

FIGS. 50A and 50B show in-gel detection of LUMIO™ labeled human kinase 96-well plate expression. The lysates from the 96-well Human Kinase Plate were run on 4-20% Tris-Glycine gels and observed under fluorescence.

FIGS. 51A-51H show in-gel detection of LUMIO™ labeled human kinase 96-well plate expression. The lysates from the 96-well Human Kinase Plate were run on 4-20% Tris-Glycine gels and stained with SIMPLYBLUE™ Safe Stain (Invitrogen Corp., Carlsbad, Calif., cat. no. LC6060).

FIGS. 52A-52I show a map of pET-DEST151 and annotated sequence data (SEQ ID NO:39). The amino acid sequences encoded by portions of this sequence are also shown. (Sequence encoded by chloramphenicol resistance gene: SEQ ID NO:40, ccdB sequence: SEQ ID NO:41, V5-2+FLASH sequence: SEQ ID NO:42, BsdR sequence: SEQ ID NO:43, AmpR sequence: SEQ ID NO:44).

FIGS. 53A-53I show a map of pENTR-DT.2/BaeIv.2/ccdB/DT and annotated sequence data (SEQ ID NO:45). The amino acid sequences encoded by portions of this sequence are also shown. (FLASH+V5-2 sequence: SEQ ID NO:46, sequence encoded by CmR gene: SEQ ID NO:47, ccdB sequence: SEQ ID NO:48, BsdR sequence: SEQ ID NO:49, AmpR sequence: SEQ ID NO:50).

FIGS. 54A-54I show a map of pET-DEST151 and annotated sequence data (SEQ ID NO:51). The amino acid sequences encoded by portions of this sequence are also shown. (His6+V5 sequence: SEQ ID NO:52, lacI sequence: SEQ ID NO:53).

FIGS. 55A-55E show a map of pENTR-DT.2 BaeIv.2 ccdB DT and annotated sequence data (SEQ ID NO:54). The amino acid sequence encoded by a portion of this sequence is also shown. (KmR sequence: SEQ ID NO:55).

FIG. 56 shows a graph of the cloning efficiency of TOPO cloning reactions as a function of the molar ratio of PCR product:vector.

### DETAILED DESCRIPTION OF THE INVENTION

### Definitions

In the description that follows, a number of terms used in recombinant nucleic acid technology are utilized extensively. In order to provide a clear and more consistent understanding of the specification and claims, including the scope to be given such terms, the following definitions are provided.

As used herein, the following is the set of 20 naturally occurring amino acids commonly found in proteins and the one and three letter codes associated with each amino acid:

| Full name | Three-letter Code | One-letter Code |
|---|---|---|
| Alanine | Ala | A |
| Arginine | Arg | R |
| Asparagine | Asn | N |
| Aspartic Acid | Asp | D |
| Cysteine | Cys | C |
| Glutamine | Gln | Q |
| Glutamic Acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

Gene: As used herein, the term “gene” refers to a nucleic acid that contains information necessary for expression of a polypeptide, protein, or untranslated RNA (e.g., rRNA, tRNA, anti-sense RNA). When the gene encodes a protein, it includes the promoter and the structural gene open reading frame sequence (ORF), as well as other sequences involved in expression of the protein. When the gene encodes an untranslated RNA, it includes the promoter and the nucleic acid that encodes the untranslated RNA.

Structural Gene: As used herein, the phrase “structural gene” refers to refers to a nucleic acid that is transcribed into messenger RNA that is then translated into a sequence of amino acids characteristic of a specific polypeptide.

Host: As used herein, the term “host” refers to any prokaryotic or eukaryotic (e.g., mammalian, insect, yeast, plant, avian, animal, etc.) organism that is a recipient of a replicable expression vector, cloning vector or any nucleic acid molecule. The nucleic acid molecule may contain, but is not limited to, a sequence of interest, a transcriptional regulatory sequence (such as a promoter, enhancer, repressor, and the like) and/or an origin of replication. As used herein, the terms “host,” “host cell,” “recombinant host” and “recombinant host cell” may be used interchangeably. For examples of such hosts, see Sambrook, et al., Molecular Cloning: A Laboratory Manual, Cold Spring Harbor Laboratory, Cold Spring Harbor, N.Y.

Transcriptional Regulatory Sequence: As used herein, the phrase “transcriptional regulatory sequence” refers to a functional stretch of nucleotides contained on a nucleic acid molecule, in any configuration or geometry, that act to regulate the transcription of (1) one or more structural genes (e.g., two, three, four, five, seven, ten, etc.) into messenger RNA or (2) one or more genes into untranslated RNA. Examples of transcriptional regulatory sequences include, but are not limited to, promoters, enhancers, repressors, operators (e.g., the tet operator), and the like.

Promoter: As used herein, a promoter is an example of a transcriptional regulatory sequence, and is specifically a nucleic acid generally described as the 5′-region of a gene located proximal to the start codon or nucleic acid that encodes untranslated RNA. The transcription of an adjacent nucleic acid segment is initiated at or near the promoter. A repressible promoter's rate of transcription decreases in response to a repressing agent. An inducible promoter's rate of transcription increases in response to an inducing agent. A constitutive promoter's rate of transcription is not specifically regulated, though it can vary under the influence of general metabolic conditions.

Target Nucleic Acid Molecule: As used herein, the phrase “target nucleic acid molecule” refers to a nucleic acid segment of interest, preferably nucleic acid that is to be acted upon using the compounds and methods of the present invention. Such target nucleic acid molecules may contain one or more (e.g., two, three, four, five, seven, ten, twelve, fifteen, twenty, thirty, fifty, etc.) genes or one or more portions of genes.

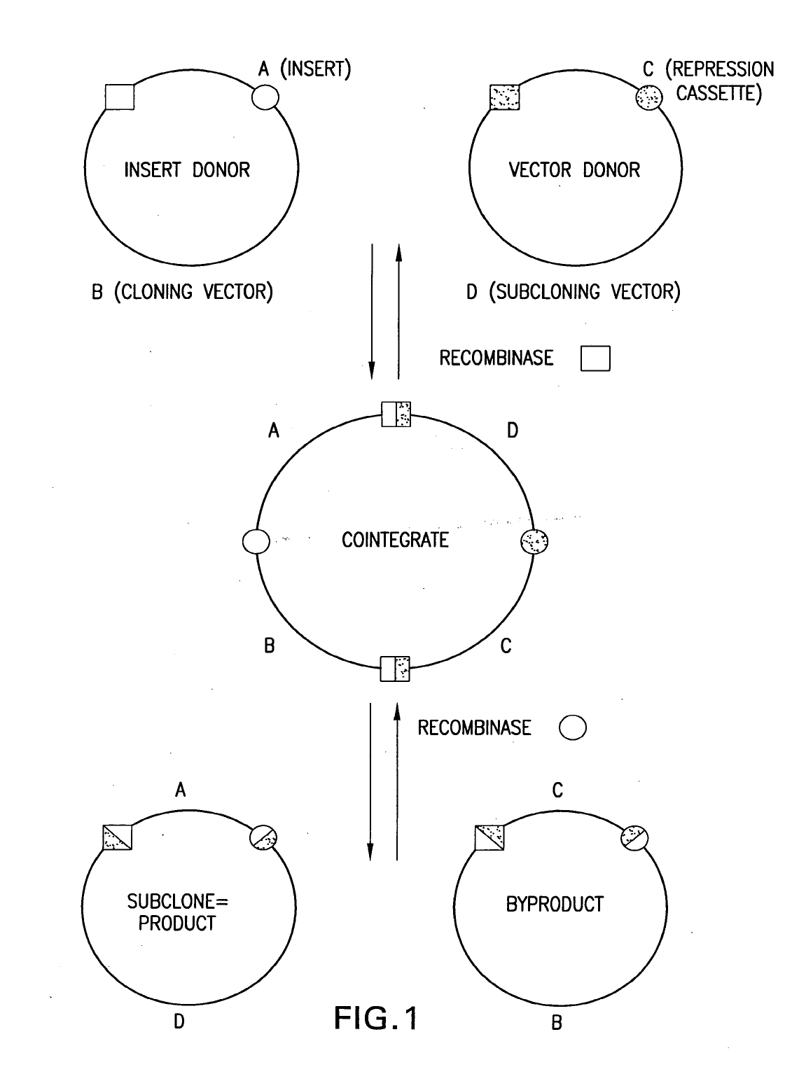

Insert Donor: As used herein, the phrase “Insert Donor” refers to one of the two parental nucleic acid molecules (e.g., RNA or DNA) of the present invention that carries an insert (see FIG. 1). The Insert Donor molecule comprises the insert flanked on both sides with recombination sites. The Insert Donor can be linear or circular. In one embodiment of the invention, the Insert Donor is a circular nucleic acid molecule, optionally supercoiled, and further comprises a cloning vector sequence outside of the recombination signals. When a population of inserts or population of nucleic acid segments are used to make the Insert Donor, a population of Insert Donors result and may be used in accordance with the invention.

Insert: As used herein, the term “insert” refers to a desired nucleic acid segment that is a part of a larger nucleic acid molecule. In many instances, the insert will be introduced into the larger nucleic acid molecule. For example, the nucleic acid segments labeled A in FIG. 1, is an insert with respect to the larger nucleic acid molecule (labeled B) shown therein. In most instances, the insert will be flanked by recombination sites, topoisomerase sites and/or other recognition sequences (e.g., at least one recognition sequence will be located at each end). In certain embodiments, however, the insert will only contain a recognition sequence on one end.

Product: As used herein, the term “Product” refers to one the desired daughter molecules comprising the A and D sequences that is produced after the second recombination event during the recombinational cloning process (see FIG. 1). The Product contains the nucleic acid that was to be cloned or subcloned. In accordance with the invention, when a population of Insert Donors are used, the resulting population of Product molecules will contain all or a portion of the population of Inserts of the Insert Donors and often will contain a representative population of the original molecules of the Insert Donors.

Byproduct: As used herein, the term “Byproduct” refers to a daughter molecule (a new clone produced after the second recombination event during the recombinational cloning process) lacking the segment that is desired to be cloned or subcloned.

Cointegrate: As used herein, the term “Cointegrate” refers to at least one recombination intermediate nucleic acid molecule of the present invention that contains both parental (starting) molecules. Cointegrates may be linear or circular. RNA and polypeptides may be expressed from cointegrates using an appropriate host cell strain, for example E. coli DB3.1 (particularly E. coli LIBRARY EFFICIENCY® DB3.1™ Competent Cells), and selecting for both selection markers found on the cointegrate molecule.

Recognition Sequence: As used herein, the phrase “recognition sequence” or “recognition site” refers to a particular sequence to which a protein, chemical compound, DNA, or RNA molecule (e.g., restriction endonuclease, a modification methylase, topoisomerases, or a recombinase) recognizes and binds. In some embodiments of the present invention, a recognition sequence may refer to a recombination site or topoisomerases site. For example, the recognition sequence for Cre recombinase is loxP which is a 34 base pair sequence comprising two 13 base pair inverted repeats (serving as the recombinase binding sites) flanking an 8 base pair core sequence (see FIG. 1 of Sauer, B., Current Opinion in Biotechnology 5:521-527 (1994)). Other examples of recognition sequences are the attB, attP, attL, and attR sequences, which are recognized by the recombinase enzyme λ Integrase. attB is an approximately 25 base pair sequence containing two 9 base pair core-type Int binding sites and a 7 base pair overlap region. attP is an approximately 240 base pair sequence containing core-type Int binding sites and arm-type Int binding sites as well as sites for auxiliary proteins integration host factor (IHF), FIS and excisionase (Xis) (see Landy, Current Opinion in Biotechnology 3:699-707 (1993)). Such sites may also be engineered according to the present invention to enhance production of products in the methods of the invention. For example, when such engineered sites lack the P1 or H1 domains to make the recombination reactions irreversible (e.g., attR or attP), such sites may be designated attR′ or attP′ to show that the domains of these sites have been modified in some way.

Recombination Proteins: As used herein, the phrase “recombination proteins” includes excisive or integrative proteins, enzymes, co-factors or associated proteins that are involved in recombination reactions involving one or more recombination sites (e.g., two, three, four, five, seven, ten, twelve, fifteen, twenty, thirty, fifty, etc.), which may be wild-type proteins (see Landy, Current Opinion in Biotechnology 3:699-707 (1993)), or mutants, derivatives (e.g., fusion proteins containing the recombination protein sequences or fragments thereof), fragments, and variants thereof. Examples of recombination proteins include Cre, Int, IHF, Xis, Flp, Fis, Hin, Gin, ΦC31, Cin, Tn3 resolvase, TndX, XerC, XerD, TnpX, Hjc, SpCCE1, and ParA.

Recombinases: As used herein, the term “recombinases” is used to refer to the protein that catalyzes strand cleavage and re-ligation in a recombination reaction. Site-specific recombinases are proteins that are present in many organisms (e.g., viruses and bacteria) and have been characterized as having both endonuclease and ligase properties. These recombinases (along with associated proteins in some cases) recognize specific sequences of bases in a nucleic acid molecule and exchange the nucleic acid segments flanking those sequences. The recombinases and associated proteins are collectively referred to as “recombination proteins” (see, e.g., Landy, A., Current Opinion in Biotechnology 3:699-707 (1993)).

Numerous recombination systems from various organisms have been described. See, e.g., Hoess, et al., Nucleic Acids Research 14(6):2287 (1986); Abremski, et al., J. Biol. Chem. 261(1):391 (1986); Campbell, J. Bacteriol. 174(23):7495 (1992); Qian, et al., J. Biol. Chem. 267(11):7794 (1992); Araki, et al., J. Mol. Biol. 225(1):25 (1992); Maeser and Kahnmann, Mol. Gen. Genet. 230:170-176) (1991); Esposito, et al., Nucl. Acids Res. 25(18):3605 (1997). Many of these belong to the integrase family of recombinases (Argos, et al., EMBO J. 5:433-440 (1986); Voziyanov, et al., Nucl. Acids Res. 27:930 (1999)). Perhaps the best studied of these are the Integrase/att system from bacteriophage λ (Landy, A. Current Opinions in Genetics and Devel. 3:699-707 (1993)), the Cre/loxP system from bacteriophage P1 (Hoess and Abremski (1990) In Nucleic Acids and Molecular Biology, vol. 4. Eds.: Eckstein and Lilley, Berlin-Heidelberg: Springer-Verlag; pp. 90-109), and the FLP/FRT system from the Saccharomyces cerevisiae 2μ circle plasmid (Broach, et al., Cell 29:227-234 (1982)).

Recombination Site: A used herein, the phrase “recombination site” refers to a recognition sequence on a nucleic acid molecule that participates in an integration/recombination reaction by recombination proteins. Recombination sites are discrete sections or segments of nucleic acid on the participating nucleic acid molecules that are recognized and bound by a site-specific recombination protein during the initial stages of integration or recombination. For example, the recombination site for Cre recombinase is loxP, which is a 34 base pair sequence comprised of two 13 base pair inverted repeats (serving as the recombinase binding sites) flanking an 8 base pair core sequence (see FIG. 1 of Sauer, B., Curr. Opin. Biotech. 5:521-527 (1994)). Other examples of recombination sites include the attB, attP, attL, and attR sequences described in U.S. provisional patent applications 60/136,744, filed May 28, 1999, and 60/188,000, filed Mar. 9, 2000, and in co-pending U.S. patent application Ser. Nos. 09/517,466 and 09/732,91—all of which are specifically incorporated herein by reference—and mutants, fragments, variants and derivatives thereof, which are recognized by the recombination protein λ Int and by the auxiliary proteins integration host factor (IHF), FIS and excisionase (Xis) (see Landy, Curr. Opin. Biotech. 3:699-707 (1993)).

Recombination sites may be added to molecules by any number of known methods. For example, recombination sites can be added to nucleic acid molecules by blunt end ligation, PCR performed with fully or partially random primers, or inserting the nucleic acid molecules into an vector using a restriction site flanked by recombination sites.

Topoisomerase recognition site. As used herein, the term “topoisomerase recognition site” or “topoisomerase site” means a defined nucleotide sequence that is recognized and bound by a site specific topoisomerase. For example, the nucleotide sequence 5′-(C/T)CCTT-3′ is a topoisomerase recognition site that is bound specifically by most poxvirus-topoisomerases, including vaccinia virus DNA topoisomerase I, which then can cleave the strand after the 3′-most thymidine of the recognition site to produce a nucleotide sequence comprising 5′-(C/T)CCTT-PO₄-TOPO, i.e., a complex of the topoisomerase covalently bound to the 3′ phosphate through a tyrosine residue in the topoisomerase (see Shuman, J. Biol. Chem. 266:11372-11379, 1991; Sekiguchi and Shuman, Nucl. Acids Res. 22:5360-5365, 1994; each of which is incorporated herein by reference; see, also, U.S. Pat. No. 5,766,891; PCT/US95/16099; and PCT/US98/12372 also incorporated herein by reference). In comparison, the nucleotide sequence 5′-GCAACTT-3′ is the topoisomerase recognition site for type IA E. coli topoisomerase III.

Recombinational Cloning: As used herein, the phrase “recombinational cloning” refers to a method, such as that described in U.S. Pat. Nos. 5,888,732; 6,143,557; 6,171,861; 6,270,969; and 6,277,608 (the contents of which are fully incorporated herein by reference), whereby segments of nucleic acid molecules or populations of such molecules are exchanged, inserted, replaced, substituted or modified, in vitro or in vivo. In many instances, the cloning method is an in vitro method.

Cloning systems that utilize recombination at defined recombination sites have been previously described in U.S. Pat. No. 5,888,732, U.S. Pat. No. 6,143,557, U.S. Pat. No. 6,171,861, U.S. Pat. No. 6,270,969, and U.S. Pat. No. 6,277,608, and in pending U.S. application Ser. No. 09/517,466 filed Mar. 2, 2000, and in published United States application no. 2002 0007051-A1, all assigned to the Invitrogen Corporation, Carlsbad, Calif., the disclosures of which are specifically incorporated herein in their entirety. In brief, the GATEWAY® Cloning System described in these patents and applications utilizes vectors that contain at least one recombination site to clone desired nucleic acid molecules in vivo or in vitro. In some embodiments, the system utilizes vectors that contain at least two different site-specific recombination sites that may be based on the bacteriophage lambda system (e.g., att1 and att2) that are mutated from the wild-type (att0) sites. Each mutated site has a unique specificity for its cognate partner att site (i.e., its binding partner recombination site) of the same type (for example attB1 with attP1, or attL1 with attR1) and will not cross-react with recombination sites of the other mutant type or with the wild-type att0 site. Different site specificities allow directional cloning or linkage of desired molecules thus providing desired orientation of the cloned molecules. Nucleic acid fragments flanked by recombination sites are cloned and subcloned using the GATEWAY® system by replacing a selectable marker (for example, ccdB) flanked by att sites on the recipient plasmid molecule, sometimes termed the Destination Vector. Desired clones are then selected by transformation of a ccdB sensitive host strain and positive selection for a marker on the recipient molecule. Similar strategies for negative selection (e.g., use of toxic genes) can be used in other organisms such as thymidine kinase (TK) in mammals and insects.

Mutating specific residues in the core region of the att site can generate a large number of different att sites. As with the att1 and att2 sites utilized in GATEWAY®, each additional mutation potentially creates a novel att site with unique specificity that will recombine only with its cognate partner att site bearing the same mutation and will not cross-react with any other mutant or wild-type att site. Novel mutated att sites (e.g., attB 1-10, attP 1-10, attR 1-10 and attL 1-10) are described in previous patent application Ser. No. 09/517,466, filed Mar. 2, 2000, which is specifically incorporated herein by reference. Other recombination sites having unique specificity (i.e., a first site will recombine with its corresponding site and will not recombine or not substantially recombine with a second site having a different specificity) may be used to practice the present invention. Examples of suitable recombination sites include, but are not limited to, loxP sites; loxP site mutants, variants or derivatives such as loxP511 (see U.S. Pat. No. 5,851,808);frt sites;frt site mutants, variants or derivatives; dif sites; dif site mutants, variants or derivatives; psi sites; psi site mutants, variants or derivatives; cer sites; and cer site mutants, variants or derivatives.

Reaction Buffers: The invention further includes reaction buffers for performing recombination reactions (e.g., LxR reaction, B×P reactions, etc.) and reaction mixtures which comprise such reaction buffer, as well as methods employing reaction buffers of the invention for performing recombination reactions and products of recombination reactions produced using such reaction buffers. The components of an enzyme mix for performing B×P reactions may include phage-encoded Integrase (Int) protein as well as Integration Host Factor (IHF). The components of an enzyme mix for performing LxR reactions may include Int, MIM, and Exisionase (Xis).

Typically, reaction buffers of the invention will contain one or more of the following components: (1) one or more buffering agent (e.g., sodium phosphate, sodium acetate, 2-(N-moropholino)-ethanesulfonic acid (MES), tris-(hydroxymethyl)aminomethane (Tris), 3-(cyclohexylamino)-2-hydroxy-1-propanesulfonic acid (CAPS), citrate, N²-hydroxyethylpiperazine-N′-2-ethanesulfonic acid (HEPES), acetate, 3-(N-morpholino)prpoanesulfonic acid (MOPS), N-tris(hydroxymethyl)methyl-3-aminopropanesulfonio acid (TAPS), etc.), (2) one or more salt (e.g., NaCl, KCl, etc.), (3) one or more chelating agent (e.g., one of more chelating agent which predominantly chelate divalent metal ions such as EDTA or EGTA), (4) one or more polyamine (e.g., spermidine, spermine, etc.), (5) one or more protein which is not typically directly involved in recombination reactions (e.g., BSA, ovalbumin, etc.), or (6) one or more diluent (e.g., water).

The concentration of the buffering agent in the reaction buffer of the invention will vary with the particular buffering agent used. Typically, the working concentration (i.e., the concentration in the reaction mixture) of the buffering agent will be from about 5 mM to about 500 mM (e.g., about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, about 100 mM, from about 5 mM to about 500 mM, from about 10 mM to about 500 mM, from about 20 mM to about 500 mM, from about 25 mM to about 500 mM, from about 30 mM to about 500 mM, from about 40 mM to about 500 mM, from about 50 mM to about 500 mM, from about 75 mM to about 500 mM, from about 100 mM to about 500 mM, from about 25 mM to about 50 mM, from about 25 mM to about 75 mM, from about 25 mM to about 100 mM, from about 25 mM to about 200 mM, from about 25 mM to about 300 mM, etc.). When Tris (e.g., Tris-HCl) is used, the Tris working concentration will typically be from about 5 mM to about 100 mM, from about 5 mM to about 75 mM, from about 10 mM to about 75 mM, from about 10 mM to about 60 mM, from about 10 mM to about 50 mM, from about 25 mM to about 50 mM, etc.

The final pH of solutions of the invention will generally be set and maintained by buffering agents present in reaction buffers of the invention. The pH of reaction buffers of the invention, and hence reaction mixtures of the invention, will vary with the particular use and the buffering agent present but will often be from about pH 5.5 to about pH 9.0 (e.g., about pH 6.0, about pH 6.5, about pH 7.0, about pH 7.1, about pH 7.2, about pH 7.3, about pH 7.4, about pH 7.5, about pH 7.6, about pH 7.7, about pH 7.8, about pH 7.9, about pH 8.0, about pH 8.1, about pH 8.5, about pH 9.0, from about pH 6.0 to about pH 8.5, from about pH 6.5 to about pH 8.5, from about pH 7.0 to about pH 8.5, from about pH 7.5 to about pH 8.5, from about pH 6.0 to about pH 8.0, from about pH 6.0 to about pH 7.7, from about pH 6.0 to about pH 7.5, from about pH 6.0 to about pH 7.0, from about pH 7.2 to about pH 7.7, from about pH 7.3 to about pH 7.7, from about pH 7.4 to about pH 7.6, from about pH 7.0 to about pH 7.4, from about pH 7.6 to about pH 8.0, from about pH 7.6 to about pH 8.5, etc.)

As indicated, one or more salts (e.g., NaCl, KCl, etc.) may be included in reaction buffers of the invention. In many instances, salts used in reaction buffers of the invention will dissociate in solution to generate at least one species which is monovalent (e.g., Na+, K+, etc.) When included in reaction buffers of the invention, salts will often be present either individually or in a combined concentration of from about 0.5 mM to about 500 mM (e.g., about 1 mM, about 2 mM, about 3 mM, about 5 mM, about 10 mM, about 12 mM, about 15 mM, about 17 mM, about 20 mM, about 22 mM, about 23 mM, about 24 mM, about 25 mM, about 27 mM, about 30 mM, about 35 mM, about 40 mM, about 45 mM, about 50 mM, about 55 mM, about 60 mM, about 64 mM, about 65 mM, about 70 mM, about 75 mM, about 80 mM, about 85 mM, about 90 mM, about 95 mM, about 100 mM, about 120 mM, about 140 mM, about 150 mM, about 175 mM, about 200 mM, about 225 mM, about 250 mM, about 275 mM, about 300 mM, about 325 mM, about 350 mM, about 375 mM, about 400 mM, from about 1 mM to about 500 mM, from about 5 mM to about 500 mM, from about 10 mM to about 500 mM, from about 20 mM to about 500 mM, from about 30 mM to about 500 mM, from about 40 mM to about 500 mM, from about 50 mM to about 500 mM, from about 60 mM to about 500 mM, from about 65 mM to about 500 mM, from about 75 mM to about 500 mM, from about 85 mM to about 500 mM, from about 90 mM to about 500 mM, from about 100 mM to about 500 mM, from about 125 mM to about 500 mM, from about 150 mM to about 500 mM, from about 200 mM to about 500 mM, from about 10 mM to about 100 mM, from about 10 mM to about 75 mM, from about 10 mM to about 50 mM, from about 20 mM to about 200 mM, from about 20 mM to about 150 mM, from about 20 mM to about 125 mM, from about 20 mM to about 100 mM, from about 20 mM to about 80 mM, from about 20 mM to about 75 mM, from about 20 mM to about 60 mM, from about 20 mM to about 50 mM, from about 30 mM to about 500 mM, from about 30 mM to about 100 mM, from about 30 mM to about 70 mM, from about 30 mM to about 50 mM, etc.).

As also indicated above, one or more agents which chelate metal ions (e.g., monovalent or divalent metal ions) with relatively high affinity may also be present in reaction buffers of the invention. Examples of compounds which chelate metal ions with relatively high affinity include ethylenediamine tetraacetic acid (EDTA), diethylenetriaminepentaacetic acid (DTPA), triethylenetetraamine hexaacetic acid (TTHA), ethylenebis(oxyethylenenitrilo)]tetraacetic acid (EGTA), and propylenetriaminepentaacetic acid (PTPA). The free acid or salt of chelating agents may be used to prepare reaction buffers of the invention.

When included in reaction buffers of the invention, chelating agents will often be present either individually or in a combined concentration of from about 0.1 mM to about 50 mM (e.g., about 0.2 mM, about 0.3 mM, about 0.5 mM, about 0.7 mM, about 0.9 mM, about 1 mM, about 2 mM, about 3 mM, about 4 mM, about 5 mM, about 6 mM, about 10 mM, about 12 mM, about 15 mM, about 17 mM, about 20 mM, about 22 mM, about 23 mM, about 24 mM, about 25 mM, about 27 mM, about 30 mM, about 35 mM, about 40 mM, about 45 mM, about 50 mM, from about 0.1 mM to about 50 mM, from about 0.5 mM to about 50 mM, from about 1 mM to about 50 mM, from about 2 mM to about 50 mM, from about 3 mM to about 50 mM, from about 0.5 mM to about 20 mM, from about 0.5 mM to about 10 mM, from about 0.5 mM to about 5 mM, from about 0.5 mM to about 2.5 mM, from about 1 mM to about 20 mM, from about 1 mM to about 10 mM, from about 1 mM to about 5 mM, from about 1 mM to about 3.4 mM, from about 0.5 mM to about 3.0 mM, from about 1 mM to about 3.0 mM, from about 1.5 mM to about 3.0 mM, from about 2 mM to about 3.0 mM, from about 0.5 mM to about 2.5 mM, from about 1 mM to about 2.5 mM, from about 1.5 mM to about 2.5 mM, from about 2 mM to about 3.0 mM, from about 2.5 mM to about 3.0 mM, from about 0.5 mM to about 2 mM, from about 0.5 mM to about 1.5 mM, from about 0.5 mM to about 1.1 mM, etc.)

Reaction buffers of the invention may also contain one or more polyamine (e.g., spermine, spermidine, protamine, polylysine, and polyethylenimine, etc.), which may be synthetic or naturally occurring. When included in reaction buffers of the invention, polyamines will often be present either individually or in a combined concentration of from about 0.1 mM to about 50 mM (e.g., about 0.2 mM, about 0.3 mM, about 0.5 mM, about 0.7 mM, about 0.9 mM, about 1 mM, about 2 mM, about 3 mM, about 4 mM, about 5 mM, about 6 mM, about 6.5 mM, about 7 mM, about 7.5 mM, about 8 mM, about 8.5 mM, about 9 mM, about 9.5 mM, about 10 mM, about 12 mM, about 15 mM, about 17 mM, about 20 mM, about 22 mM, about 23 mM, about 24 mM, about 25 mM, about 27 mM, about 30 mM, about 35 mM, about 40 mM, about 45 mM, about 50 mM, from about 0.1 mM to about 50 mM, from about 0.5 mM to about 50 mM, from about 1 mM to about 50 mM, from about 2 mM to about 50 mM, from about 3 mM to about 50 mM, from about 0.5 mM to about 20 mM, from about 0.5 mM to about 10 mM, from about 0.5 mM to about 5 mM, from about 0.5 mM to about 2.5 mM, from about 1 mM to about 20 mM, from about 1 mM to about 10 mM, from about 1 mM to about 5 mM, from about 1 mM to about 3.4 mM, from about 0.5 mM to about 3.0 mM, from about 1 mM to about 3.0 mM, from about 1.5 mM to about 3.0 mM, from about 2 mM to about 3.0 mM, from about 0.5 mM to about 2.5 mM, from about 1 mM to about 2.5 mM, from about 1.5 mM to about 2.5 mM, from about 2 mM to about 3.0 mM, from about 2.5 mM to about 3.0 mM, from about 0.5 mM to about 2 mM, from about 0.5 mM to about 1.5 mM, from about 0.5 mM to about 1.1 mM, from about 7.6 mM to about 20 mM, from about 7.7 mM to about 20 mM, from about 7.8 mM to about 20 mM, from about 8.0 mM to about 20 mM, from about 8.1 mM to about 20 mM, from about 8.2 mM to about 20 mM, from about 8.3 mM to about 20 mM, from about 8.4 mM to about 20 mM, from about 8.5 mM to about 20 mM, from about 9.0 mM to about 20 mM, from about 10.0 mM to about 20 mM, from about 12.0 mM to about 20 mM, from about 7.6 mM to about 50 mM, from about 8.0 mM to about 50 mM, etc.). For example, reaction buffers of the invention may contain spermidine at a concentration of from about 7.6 mM to about 20 mM, from about 7.7 mM to about 20 mM, from about 7.8 mM to about 20 mM, from about 8.0 mM to about 20 mM, from about 8.1 mM to about 20 mM, from about 8.2 mM to about 20 mM, from about 8.3 mM to about 20 mM, from about 8.4 mM to about 20 mM, from about 8.5 mM to about 20 mM, from about 9.0 mM to about 20 mM, from about 10.0 mM to about 20 mM, from about 12.0 mM to about 20 mM, from about 7.6 mM to about 50 mM, from about 8.0 mM to about 50 mM, etc.

Reaction buffers of the invention may also contain one or more protein which is not typically directly involved in recombination reactions (e.g., bovine serum albumin (BSA); ovalbumin; immunoglobins, such as IgE, IgG, IgD; etc.). When included in reaction buffers of the invention, such proteins will often be present either individually or in a combined concentration of from about 0.1 mg/ml to about 50 mg/ml (e.g., about 0.1 mg/ml, about 0.2 mg/ml, about 0.3 mg/ml, about 0.4 mg/ml, about 0.5 mg/ml, about 0.6 mg/ml, about 0.7 mg/ml, about 0.8 mg/ml, about 0.9 mg/ml, about 1.0 mg/ml, about 1.1 mg/ml, about 1.3 mg/ml, about 1.5 mg/ml, about 1.7 mg/ml, about 2.0 mg/ml, about 2.5 mg/ml, about 3.5 mg/ml, about 5.0 mg/ml, about 7.5 mg/ml, about 10 mg/ml, about 15 mg/ml, about 20 mg/ml, about 25 mg/ml, about 30 mg/ml, about 35 mg/ml, about 40 mg/ml, from about 0.5 mg/ml to about 30 mg/ml, from about 0.75 mg/ml to about 30 mg/ml, from about 1.0 mg/ml to about 30 mg/ml, from about 2.0 mg/ml to about 30 mg/ml, from about 3.0 mg/ml to about 30 mg/ml, from about 4.0 mg/ml to about 30 mg/ml, from about 5.0 mg/ml to about 30 mg/ml, from about 7.5 mg/ml to about 30 mg/ml, from about 10 mg/ml to about 30 mg/ml, from about 15 mg/ml to about 30 mg/ml, from about 0.5 mg/ml to about 20 mg/ml, from about 0.5 mg/ml to about 10 mg/ml, from about 0.5 mg/ml to about 5 mg/ml, from about 0.5 mg/ml to about 2 mg/ml, from about 0.5 mg/ml to about 1 mg/ml, from about 1 mg/ml to about 10 mg/ml, from about 1 mg/ml to about 5 mg/ml, from about 1 mg/ml to about 2 mg/ml, etc.).

Examples of reaction buffers of the invention include the following: (1) 50 mM Tris-HCl (pH 7.5), 1 mM EDTA, 1 mg/ml BSA, 64 mM NaCl, 8 mM spermidine; (2) 50 mM Tris-HCl (pH 7.5), 1 mM EDTA, 1 mg/ml BSA, 64 mM NaCl, 10 mM spermidine; (3) 50 mM Tris-HCl (pH 7.5), 1 mM EDTA, 1 mg/ml BSA, 64 mM NaCl, 12 mM spermidine; (4) 50 mM Tris-HCl (pH 7.5), 1 mM EDTA, 1 mg/ml BSA, 75 mM NaCl, 8 mM spermidine; (5) 50 mM Tris-HCl (pH 7.5), 1 mM EDTA, 1 mg/ml BSA, 64 mM NaCl, 15 mM spermidine; (6) 25 mM Tris-HCl (pH 7.5), 1 mM EDTA, 1 mg/ml BSA, 64 mM NaCl, 8 mM spermidine; (7) 50 mM Tris-HCl (pH 7.5), 1 mM EDTA, 2 mg/ml BSA, 64 mM NaCl, 8 mM spermidine; (8) 25 mM Tris-HCl (pH 7.5), 5 mM EDTA, 1 mg/ml BSA, 64 mM NaCl, 8 mM spermidine; (9) 25 mM Tris-HCl (pH 7.5), 1 mM EDTA, 2 mg/ml BSA, 64 mM NaCl, 8 mM spermidine; (10) 100 mM Tris-HCl (pH 7.5), 1 mM EDTA, 1 mg/ml BSA, 64 mM NaCl, 10 mM spermidine; (11) 75 mM Tris-HCl (pH 7.5), 1 mM EDTA, 1 mg/ml BSA, 65 mM NaCl, 8 mM spermidine; (12) 50 mM Tris-HCl (pH 7.5), 1 mM EDTA, 1 mg/ml BSA, 64 mM NaCl, 8 mM spermine; (13) 50 mM Tris-HCl (pH 7.5), 1 mM EDTA, 1 mg/ml BSA, 65 mM NaCl, 8 mM spermidine; (14) 50 mM Tris-HCl (pH 7.5), 1 mM EDTA, 1 mg/ml BSA, 64 mM KCl, 8 mM spermidine; and (15) 75 mM Tris-HCl (pH 7.5), 1 mM EDTA, 1 mg/ml BSA, 64 mM KCl, 8 mM spermidine.

Reaction buffers of the invention may be prepared as concentrated solutions which are diluted to a working concentration for final use. For example, a reaction buffer of the invention may be prepared as a 5× concentrate with the following working concentrations of components being 50 mM Tris-HCl (pH 7.5), 1 mM EDTA, 1 mg/ml BSA, 64 mM NaCl, 8 mM spermidine. Such a 5× solution would contain 200 mM Tris-HCl (pH 7.5), 5 mM EDTA, 5 mg/ml BSA, 325 mM NaCl, and 40 mM spermidine. As another example, a reaction buffer of the invention for performing a LR reaction may be prepared as a 5× concentrate with the following working concentrations of components being 30 mM Tris-HCl (pH 7.4), 4.05 mM EDTA, 0.84 mg/ml BSA, 27.6 mM NaCl, 4.5 mM spermidine, 10% glycerol, 4.4 μg/ml Int, 1.5 μg/ml IHF, and 0.82 μg/ml Xis. Such a 5× solution would contain 150 mM Tris-HCl (pH 7.4), 20.25 mM EDTA, 4.2 mg/ml BSA, 138 mM NaCl, 22.5 mM spermidine, 50% glycerol, 22 μg/ml Int, 7.5 μg/ml IHF, and 4.1 μg/ml Xis. As yet another example, a reaction buffer of the invention for performing a BP reaction may be prepared as a 5× concentrate with the following working concentrations of components being 25 mM Tris-HCl (pH 7.4), 5 mM EDTA, 1 mg/ml BSA, 22 mM NaCl, 5 mM spermidine, 0.2 mM dithiothreitol (DTT), 0.0005% TRITON X-100™, 10% glycerol, 6.6 μg/ml Int, and 4 μg/ml IHF. Such a 5× solution would contain 125 mM Tris-HCl (pH 7.4), 25 mM EDTA, 4.2 mg/ml BSA, 110 mM NaCl, 25 mM spermidine, 1 mM DTT, 0.0025% TRITON X-100™, 50% glycerol, 33 μg/ml Int, and 20 μg/ml IHF. Thus, a 5:1 dilution is required to bring such 5× solutions to a working concentration. Reaction buffers of the invention may be prepared, for examples, as a 2×, a 3×, a 4×, a 5×, a 6×, a 7×, a 8×, a 9×, a 10×, etc. solutions. One major limitation on the fold concentration of such solutions is that, when compounds reach particular concentrations in solution, precipitation occurs. Thus, concentrated reaction buffers will generally be prepared such that the concentrations of the various components are low enough so that precipitation of buffer components will not occur. As one skilled in the art would recognize, the upper limit of concentration which is feasible for each solution will vary with the particular solution and the components present.

In some instances, the recombination reaction mixture buffer contain all of the components necessary for performing the reaction except for the nucleic acid. For example, recombination reaction mixtures will be started by adding the nucleic acids to be recombined, which may be added in one solution or in two different solutions. In many instances, the nucleic acids which are added to the reaction mixture buffer will be in water.

In many instances, reaction buffers of the invention will be provided in sterile form. Sterilization may be performed on the individual components of reaction buffers prior to mixing or on reaction buffers after they are prepared. Sterilization of such solutions may be performed by any suitable means including autoclaving or ultrafiltration.

Nucleic acid molecules used in methods of the invention, as well as those prepared by methods of the invention, may be dissolved in an aqueous buffer and added to the reaction mixture. One suitable set of conditions is 4 μl CLONASE™ enzyme mixture (e.g., Invitrogen Corporation, Cat. Nos. 11791-019 and 11789-013), 4 μl 5× reaction buffer and nucleic acid and water to a final volume of 20 μl. This will typically result in the inclusion of about 200 ng of Int and about 80 ng of IHF in a 20 μl BP reaction and about 150 ng Int, about 25 ng IHF and about 30 ng Xis in a 20 μl LR reaction.

Additional suitable sets of conditions include the use of smaller reaction volumes, for example, 2 μl CLONASE™ enzyme mixture (e.g., Invitrogen Corporation, Cat. Nos. 11791-019 and 11789-013), 2 μl 5× reaction buffer and nucleic acid and water to a final volume of 10 μl. In other embodiments, a suitable set of conditions includes 2 μl CLONASE™ enzyme mixture (e.g., Invitrogen Corporation, Cat. Nos. 11791-019 and 11789-013), 1 μl 10× reaction buffer and nucleic acid and water to a final volume of 10 μl.

Proteins for conducting an LR reaction may be stored in a suitable buffer, for example, LR Storage Buffer, which may comprise about 50 mM Tris at about pH 7.5, about 50 mM NaCl, about 0.25 mM EDTA, about 2.5 mM spermidine, and about 0.2 mg/ml BSA. When stored, proteins for an LR reaction may be stored at a concentration of about 37.5 ng/μl INT, 10 ng/μl IHF and 15 ng/μl XIS. Proteins for conducting a BP reaction may be stored in a suitable buffer, for example, BP Storage Buffer, which may comprise about 25 mM Tris at about pH 7.5, about 22 mM NaCl, about 5 mM EDTA, about 5 mM spermidine, about 1 mg/ml BSA, and about 0.0025% TRITON X-100™. When stored, proteins for an BP reaction may be stored at a concentration of about 37.5 ng/μl INT and 20 ng/μl IHF. One skilled in the art will recognize that enzymatic activity may vary in different preparations of enzymes. The amounts suggested above may be modified to adjust for the amount of activity in any specific preparation of enzymes.

A suitable 5× reaction buffer for conducting recombination reactions may comprise 100 mM Tris pH 7.5, 88 mM NaCl, 20 mM EDTA, 20 mM spermidine, and 4 mg/ml BSA. Thus, in a recombination reaction, the final buffer concentrations may be 20 mM Tris pH 7.5, 17.6 mM NaCl, 4 mM EDTA, 4 mM spermidine, and 0.8 mg/ml BSA. Those skilled in the art will appreciate that the final reaction mixture may incorporate additional components added with the reagents used to prepare the mixture, for example, a BP reaction may include 0.005% TRITON X-100™ incorporated from the BP Clonase™.

In additional embodiments, a 10× reaction buffer for conducting recombination reactions may be prepared and comprise 200 mM Tris pH 7.5, 176 mM NaCl, 40 mM EDTA, 40 mM spermidine, and 8 mg/ml BSA. Thus, in a recombination reaction, the final buffer concentrations may be 20 mM Tris pH 7.5, 17.6 mM NaCl, 4 mM EDTA, 4 mM spermidine, and 0.8 mg/ml BSA. Those skilled in the art will appreciate that the final reaction mixture may incorporate additional components added with the reagents used to prepare the mixture, for example, a BP reaction may include 0.01% TRITON X-100™ incorporated from the BP Clonase™.

In particular embodiments, particularly those in which attL sites are to be recombined with attR sites, the final reaction mixture may include about 50 mM Tris HCl, pH 7.5, about 1 mM EDTA, about 1 mg/ml BSA, about 75 mM NaCl and about 7.5 mM spermidine in addition to recombination enzymes and the nucleic acids to be combined. In other embodiments, particularly those in which an attB site is to be recombined with an attP site, the final reaction mixture may include about 25 mM Tris HCl, pH 7.5, about 5 mM EDTA, about 1 mg/ml bovine serum albumin (BSA), about 22 mM NaCl, and about 5 mM spermidine.

In some embodiments, particularly those in which attL sites are to be recombined with attR sites, the final reaction mixture may include about 40 mM Tris HCl, pH 7.5, about 1 mM EDTA, about 1 mg/ml BSA, about 64 mM NaCl and about 8 mM spermidine in addition to recombination enzymes and the nucleic acids to be combined. One of skill in the art will appreciate that the reaction conditions may be varied somewhat without departing from the invention. For example, the pH of the reaction may be varied from about 7.0 to about 8.0; the concentration of buffer may be varied from about 25 mM to about 100 mM; the concentration of EDTA may be varied from about 0.5 mM to about 2 mM; the concentration of NaCl may be varied from about 25 mM to about 150 mM; and the concentration of BSA may be varied from 0.5 mg/ml to about 5 mg/ml. In other embodiments, particularly those in which an attB site is to be recombined with an attP site, the final reaction mixture may include about 25 mM Tris HCl, pH 7.5, about 5 mM EDTA, about 1 mg/ml bovine serum albumin (BSA), about 22 mM NaCl, about 5 mM spermidine and about 0.005% detergent (e.g., TRITON X-100™).

In other embodiments, the recombination reactions may be prepared using a buffer which performs the functions of both the storage and reaction buffers in one. Suitably, in such embodiments, this buffer may comprise between about 100-200 mM Tris pH 7.5, between about 88-176 mM NaCl, between about 20-40 mM EDTA, between about 20-40 mM spermidine, and between about 4-8 mg/ml BSA. Those skilled in the art will appreciate that the final reaction mixture may incorporate additional components added with the reagents used to prepare the mixture, for example, a BP reaction may include between about 0.005-0.01% TRITON X-100™ incorporated from the BP Clonase™. These combination buffers would also include proteins for conducting an LR or a BP reaction. When stored, proteins for an LR reaction may be stored at a concentration of between about 37.5-75 ng/μl INT, between about 10-20 ng/μl IHF and between about 15-30 ng/μl XIS; proteins for an BP reaction may be stored at a concentration of between about 37.5-75 ng/μl INT and between about 20-40 ng/μl IHF.

The amount of nucleic acid which is the subject of recombination reactions may vary considerably. Typically, the amount of nucleic acid present in a 10 μl final reaction mixture will be between 50 and 500 ng, 10 and 500 ng, 25 and 500 ng, 75 and 500 ng, 100 and 500 ng, 200 and 500 ng, 300 and 500 ng, 50 and 300 ng, 50 and 250 ng, 250 and 500 ng, or 50 and 400 ng. Further, the nucleic acids which are the subject of the recombination reaction need not be present in equal amounts. For example, when two nucleic acids are to be recombined, they may be present in an amount defined by a ratio of 1:1.1, 1:1.2, 1:1.3, 1:1.4, 1:1.5, 1:2.0. 1:2.5, or 1:3.0.

Repression Cassette: As used herein, the phrase “repression cassette” refers to a nucleic acid segment that contains a repressor or a selectable marker present in the subcloning vector.

Selectable Marker: As used herein, the phrase “selectable marker” refers to a nucleic acid segment that allows one to select for or against a molecule (e.g., a replicon) or a cell that contains it and/or permits identification of a cell or organism that contains or does not contain the nucleic acid segment. Frequently, selection and/or identification occur under particular conditions and do not occur under other conditions.

Markers can encode an activity, such as, but not limited to, production of RNA, peptide, or protein, or can provide a binding site for RNA, peptides, proteins, inorganic and organic compounds or compositions and the like. Examples of selectable markers include but are not limited to: (1) nucleic acid segments that encode products that provide resistance against otherwise toxic compounds (e.g., antibiotics); (2) nucleic acid segments that encode products that are otherwise lacking in the recipient cell (e.g., tRNA genes, auxotrophic markers); (3) nucleic acid segments that encode products that suppress the activity of a gene product; (4) nucleic acid segments that encode products that can be readily identified (e.g., phenotypic markers such as β-lactamase, β-galactosidase, green fluorescent protein (GFP), yellow flourescent protein (YFP), red fluorescent protein (RFP), cyan fluorescent protein (CFP), and cell surface proteins); (5) nucleic acid segments that bind products that are otherwise detrimental to cell survival and/or function; (6) nucleic acid segments that otherwise inhibit the activity of any of the nucleic acid segments described in Nos. 1-5 above (e.g., antisense oligonucleotides); (7) nucleic acid segments that bind products that modify a substrate (e.g., restriction endonucleases); (8) nucleic acid segments that can be used to isolate or identify a desired molecule (e.g., specific protein binding sites); (9) nucleic acid segments that encode a specific nucleotide sequence that can be otherwise non-functional (e.g., for PCR amplification of subpopulations of molecules); (10) nucleic acid segments that, when absent, directly or indirectly confer resistance or sensitivity to particular compounds; and/or (11) nucleic acid segments that encode products that either are toxic (e.g., Diphtheria toxin) or convert a relatively non-toxic compound to a toxic compound (e.g., Herpes simplex thymidine kinase, cytosine deaminase) in recipient cells; (12) nucleic acid segments that inhibit replication, partition or heritability of nucleic acid molecules that contain them; and/or (13) nucleic acid segments that encode conditional replication functions, e.g., replication in certain hosts or host cell strains or under certain environmental conditions (e.g., temperature, nutritional conditions, etc.).

Selection and/or identification may be accomplished using techniques well known in the art. For example, a selectable marker may confer resistance to an otherwise toxic compound and selection may be accomplished by contacting a population of host cells with the toxic compound under conditions in which only those host cells containing the selectable marker are viable. In another example, a selectable marker may confer sensitivity to an otherwise benign compound and selection may be accomplished by contacting a population of host cells with the benign compound under conditions in which only those host cells that do not contain the selectable marker are viable. A selectable marker may make it possible to identify host cells containing or not containing the marker by selection of appropriate conditions. In one aspect, a selectable marker may enable visual screening of host cells to determine the presence or absence of the marker. For example, a selectable marker may alter the color and/or fluorescence characteristics of a cell containing it. This alteration may occur in the presence of one or more compounds, for example, as a result of an interaction between a polypeptide encoded by the selectable marker and the compound (e.g., an enzymatic reaction using the compound as a substrate). Such alterations in visual characteristics can be used to physically separate the cells containing the selectable marker from those not contain it by, for example, fluorescent activated cell sorting (FACS).

Multiple selectable markers may be simultaneously used to distinguish various populations of cells. For example, a nucleic acid molecule of the invention may have multiple selectable markers, one or more of which may be removed from the nucleic acid molecule by a suitable reaction (e.g., a recombination reaction). After the reaction, the nucleic acid molecules may be introduced into a host cell population and those host cells comprising nucleic acid molecules having all of the selectable markers may be distinguished from host cells comprising nucleic acid molecules in which one or more selectable markers have been removed (e.g., by the recombination reaction). For example, a nucleic acid molecule of the invention may have a blasticidin resistance marker outside a pair of recombination sites and a β-lactamase encoding selectable marker inside the recombination sites. After a recombination reaction and introduction of the reaction mixture into a cell population, cells comprising any nucleic acid molecule can be selected for by contacting the cell population with blasticidin. Those cell comprising a nucleic acid molecule that has undergone a recombination reaction can be distinguished from those containing an unreacted nucleic acid molecules by contacting the cell population with a fluorogenic β-lactamase substrate as described below and observing the fluorescence of the cell population. Optionally, the desired cells can be physically separated from undesirable cells, for example, by FACS.

Selection Scheme: As used herein, the phrase “selection scheme” refers to any method that allows selection, enrichment, or identification of a desired nucleic acid molecules or host cells containing them (in particular Product or Product(s) from a mixture containing an Entry Clone or Vector, a Destination Vector, a Donor Vector, an Expression Clone or Vector, any intermediates (e.g., a Cointegrate or a replicon), and/or Byproducts). In one aspect, selection schemes of the invention rely on one or more selectable markers. The selection schemes of one embodiment have at least two components that are either linked or unlinked during recombinational cloning. One component is a selectable marker. The other component controls the expression in vitro or in vivo of the selectable marker, or survival of the cell (or the nucleic acid molecule, e.g., a replicon) harboring the plasmid carrying the selectable marker. Generally, this controlling element will be a repressor or inducer of the selectable marker, but other means for controlling expression or activity of the selectable marker can be used. Whether a repressor or activator is used will depend on whether the marker is for a positive or negative selection, and the exact arrangement of the various nucleic acid segments, as will be readily apparent to those skilled in the art. In some embodiments, the selection scheme results in selection of, or enrichment for, only one or more desired nucleic acid molecules (such as Products). As defined herein, selecting for a nucleic acid molecule includes (a) selecting or enriching for the presence of the desired nucleic acid molecule (referred to as a “positive selection scheme”), and (b) selecting or enriching against the presence of nucleic acid molecules that are not the desired nucleic acid molecule (referred to as a “negative selection scheme”).

In one embodiment, the selection schemes (which can be carried out in reverse) will take one of three forms, which will be discussed in terms of FIG. 1. The first, exemplified herein with a selectable marker and a repressor therefore, selects for molecules having segment D and lacking segment C. The second selects against molecules having segment C and for molecules having segment D. Possible embodiments of the second form would have a nucleic acid segment carrying a gene toxic to cells into which the in vitro reaction products are to be introduced. A toxic gene can be a nucleic acid that is expressed as a toxic gene product (a toxic protein or RNA), or can be toxic in and of itself. (In the latter case, the toxic gene is understood to carry its classical definition of “heritable trait.”)

Examples of such toxic gene products are well known in the art, and include, but are not limited to, restriction endonucleases (e.g., DpnI, Nla3, etc.); apoptosis-related genes (e.g., ASK1 or members of the bcl-2/ced-9 family); retroviral genes; including those of the human immunodeficiency virus (HIV); defensins such as NP-1; inverted repeats or paired palindromic nucleic acid sequences; bacteriophage lytic genes such as those from φX174 or bacteriophage T4; antibiotic sensitivity genes such as rpsL; antimicrobial sensitivity genes such as pheS; plasmid killer genes' eukaryotic transcriptional vector genes that produce a gene product toxic to bacteria, such as GATA-1; genes that kill hosts in the absence of a suppressing function, e.g., kicB, ccdB, ΦX174 E (Liu, Q., et al., Curr. Biol. 8:1300-1309 (1998)); and other genes that negatively affect replicon stability and/or replication. A toxic gene can alternatively be selectable in vitro, e.g., a restriction site.

Many genes coding for restriction endonucleases operably linked to inducible promoters are known, and may be used in the present invention (see, e.g., U.S. Pat. Nos. 4,960,707 (DpnI and DpnII); 5,082,784 and 5,192,675 (KpnI); 5,147,800 (NgoAIII and NgoAI); 5,179,015 (FspI and HaeIII): 5,200,333 (HaeII and TaqI); 5,248,605 (HpaII); 5,312,746 (ClaI); 5,231,021 and 5,304,480 (XhoI and XhoII); 5,334,526 (Alul); 5,470,740 (NsiI); 5,534,428 (SstI/SacI); 5,202,248 (NcoI); 5,139,942 (NdeI); and 5,098,839 (PacI). (See also Wilson, G.G., Nucl. Acids Res. 19:2539-2566 (1991); and Lunnen, K. D., et al., Gene 74:25-32 (1988)).

In the second form, segment D carries a selectable marker. The toxic gene would eliminate transformants harboring the Vector Donor, Cointegrate, and Byproduct molecules, while the selectable marker can be used to select for cells containing the Product and against cells harboring only the Insert Donor.

The third form selects for cells that have both segments A and D in cis on the same molecule, but not for cells that have both segments in trans on different molecules. This could be embodied by a selectable marker that is split into two inactive fragments, one each on segments A and D.

The fragments are so arranged relative to the recombination sites that when the segments are brought together by the recombination event, they reconstitute a functional selectable marker. For example, the recombinational event can link a promoter with a structural nucleic acid molecule (e.g., a gene), can link two fragments of a structural nucleic acid molecule, or can link nucleic acid molecules that encode a heterodimeric gene product needed for survival, or can link portions of a replicon.

Site-Specific Recombinase: As used herein, the phrase “site-specific recombinase” refers to a type of recombinase that typically has at least the following four activities (or combinations thereof): (1) recognition of specific nucleic acid sequences; (2) cleavage of said sequence or sequences; (3) topoisomerase activity involved in strand exchange; and (4) ligase activity to reseal the cleaved strands of nucleic acid (see Sauer, B., Current Opinions in Biotechnology 5:521-527 (1994)). Conservative site-specific recombination is distinguished from homologous recombination and transposition by a high degree of sequence specificity for both partners. The strand exchange mechanism involves the cleavage and rejoining of specific nucleic acid sequences in the absence of DNA synthesis (Landy, A. (1989) Ann. Rev. Biochem. 58:913-949).

Suppressor tRNA. As used herein, the phrase “suppressor tRNA” is used to indicate a tRNA molecule that results in the incorporation of an amino acid in a polypeptide in a position corresponding to a stop codon in the mRNA being translated.

Homologous Recombination: As used herein, the phrase “homologous recombination” refers to the process in which nucleic acid molecules with similar nucleotide sequences associate and exchange nucleotide strands. A nucleotide sequence of a first nucleic acid molecule that is effective for engaging in homologous recombination at a predefined position of a second nucleic acid molecule will therefore have a nucleotide sequence that facilitates the exchange of nucleotide strands between the first nucleic acid molecule and a defined position of the second nucleic acid molecule. Thus, the first nucleic acid will generally have a nucleotide sequence that is sufficiently complementary to a portion of the second nucleic acid molecule to promote nucleotide base pairing.

Homologous recombination requires homologous sequences in the two recombining partner nucleic acids but does not require any specific sequences. As indicated above, site-specific recombination that occurs, for example, at recombination sites such as att sites, is not considered to be “homologous recombination,” as the phrase is used herein.

Vector: As used herein, the term “vector” refers to a nucleic acid molecule (e.g., DNA) that provides a useful biological or biochemical property to an insert. Examples include plasmids, phages, autonomously replicating sequences (ARS), centromeres, and other sequences that are able to replicate or be replicated in vitro or in a host cell, or to convey a desired nucleic acid segment to a desired location within a host cell. A vector can have one or more recognition sites (e.g., two, three, four, five, seven, ten, etc. recombination sites, restriction sites, and/or topoisomerases sites) at which the sequences can be manipulated in a determinable fashion without loss of an essential biological function of the vector, and into which a nucleic acid fragment can be spliced in order to bring about its replication and cloning. Vectors can further provide primer sites (e.g., for PCR), transcriptional and/or translational initiation and/or regulation sites, recombinational signals, replicons, selectable markers, etc. Clearly, methods of inserting a desired nucleic acid fragment that do not require the use of recombination, transpositions or restriction enzymes (such as, but not limited to, uracil N-glycosylase (UDG) cloning of PCR fragments (U.S. Pat. Nos. 5,334,575 and 5,888,795, both of which are entirely incorporated herein by reference), T:A cloning, and the like) can also be applied to clone a fragment into a cloning vector to be used according to the present invention. The cloning vector can further contain one or more selectable markers (e.g., two, three, four, five, seven, ten, etc.) suitable for use in the identification of cells transformed with the cloning vector.

Subcloning Vector: As used herein, the phrase “subcloning vector” refers to a cloning vector comprising a circular or linear nucleic acid molecule that includes, in many instances, an appropriate replicon. In the present invention, the subcloning vector (segment D in FIG. 1) can also contain functional and/or regulatory elements that are desired to be incorporated into the final product to act upon or with the cloned nucleic acid insert (segment A in FIG. 1). The subcloning vector can also contain a selectable marker (e.g., DNA).

Vector Donor: As used herein, the phrase “Vector Donor” refers to one of the two parental nucleic acid molecules (e.g., RNA or DNA) of the present invention that carries the nucleic acid segments comprising the nucleic acid vector that is to become part of the desired Product. The Vector Donor comprises a subcloning vector D (or it can be called the cloning vector if the Insert Donor does not already contain a cloning vector) and a segment C flanked by recombination sites (see FIG. 1). Segments C and/or D can contain elements that contribute to selection for the desired Product daughter molecule, as described above for selection schemes. The recombination signals can be the same or different, and can be acted upon by the same or different recombinases. In addition, the Vector Donor can be linear or circular.

Primer: As used herein, the term “primer” refers to a single stranded or double stranded oligonucleotide that is extended by covalent bonding of nucleotide monomers during amplification or polymerization of a nucleic acid molecule (e.g., a DNA molecule). In one aspect, the primer may be a sequencing primer (for example, a universal sequencing primer). In another aspect, the primer may comprise a recombination site or portion thereof.

Adapter: As used herein, the term “adapter” refers to an oligonucleotide or nucleic acid fragment or segment (e.g., DNA) that comprises one or more recombination sites and/or topoisomerase site (or portions of such sites) that can be added to a circular or linear Insert Donor molecule as well as to other nucleic acid molecules described herein. When using portions of sites, the missing portion may be provided by the Insert Donor molecule. Such adapters may be added at any location within a circular or linear molecule, although the adapters are typically added at or near one or both termini of a linear molecule. Adapters may be positioned, for example, to be located on both sides (flanking) a particular nucleic acid molecule of interest. In accordance with the invention, adapters may be added to nucleic acid molecules of interest by standard recombinant techniques (e.g., restriction digest and ligation). For example, adapters may be added to a circular molecule by first digesting the molecule with an appropriate restriction enzyme, adding the adapter at the cleavage site and reforming the circular molecule that contains the adapter(s) at the site of cleavage. In other aspects, adapters may be added by homologous recombination, by integration of RNA molecules, and the like. Alternatively, adapters may be ligated directly to one or more terminus or both termini of a linear molecule thereby resulting in linear molecule(s) having adapters at one or both termini. In one aspect of the invention, adapters may be added to a population of linear molecules, (e.g., a cDNA library or genomic DNA that has been cleaved or digested) to form a population of linear molecules containing adapters at one terminus or both termini of all or substantial portion of said population.

Adapter-Primer: As used herein, the phrase “adapter-primer” refers to a primer molecule that comprises one or more recombination sites (or portions of such recombination sites) that can be added to a circular or to a linear nucleic acid molecule described herein. When using portions of recombination sites, the missing portion may be provided by a nucleic acid molecule (e.g., an adapter) of the invention. Such adapter-primers may be added at any location within a circular or linear molecule, although the adapter-primers may be added at or near one or both termini of a linear molecule. Such adapter-primers may be used to add one or more recombination sites or portions thereof to circular or linear nucleic acid molecules in a variety of contexts and by a variety of techniques, including but not limited to amplification (e.g., PCR), ligation (e.g., enzymatic or chemical/synthetic ligation), recombination (e.g., homologous or non-homologous (illegitimate) recombination) and the like.

Template: As used herein, the term “template” refers to a double stranded or single stranded nucleic acid molecule that is to be amplified, synthesized or sequenced. In the case of a double-stranded DNA molecule, denaturation of its strands to form a first and a second strand may be performed before these molecules may be amplified, synthesized or sequenced, or the double stranded molecule may be used directly as a template. For single stranded templates, a primer complementary to at least a portion of the template hybridizes under appropriate conditions and one or more polypeptides having polymerase activity (e.g., two, three, four, five, or seven DNA polymerases and/or reverse transcriptases) may then synthesize a molecule complementary to all or a portion of the template. Alternatively, for double stranded templates, one or more transcriptional regulatory sequences (e.g., two, three, four, five, seven or more promoters) may be used in combination with one or more polymerases to make nucleic acid molecules complementary to all or a portion of the template. The newly synthesized molecule, according to the invention, may be of equal or shorter length compared to the original template. Mismatch incorporation or strand slippage during the synthesis or extension of the newly synthesized molecule may result in one or a number of mismatched base pairs. Thus, the synthesized molecule need not be exactly complementary to the template. Additionally, a population of nucleic acid templates may be used during synthesis or amplification to produce a population of nucleic acid molecules typically representative of the original template population.

Incorporating: As used herein, the term “incorporating” means becoming a part of a nucleic acid (e.g., DNA) molecule or primer.

Library: As used herein, the term “library” refers to a collection of nucleic acid molecules (circular or linear). In one embodiment, a library may comprise a plurality of nucleic acid molecules (e.g., two, three, four, five, seven, ten, twelve, fifteen, twenty, thirty, fifty, one hundred, two hundred, five hundred one thousand, five thousand, or more), that may or may not be from a common source organism, organ, tissue, or cell. In another embodiment, a library is representative of all or a portion or a significant portion of the nucleic acid content of an organism (a “genomic” library), or a set of nucleic acid molecules representative of all or a portion or a significant portion of the expressed nucleic acid molecules (a cDNA library or segments derived there from) in a cell, tissue, organ or organism. A library may also comprise nucleic acid molecules having random sequences made by de novo synthesis, mutagenesis of one or more nucleic acid molecules, and the like. Such libraries may or may not be contained in one or more vectors (e.g., two, three, four, five, seven, ten, twelve, fifteen, twenty, thirty, fifty, etc.).

Amplification: As used herein, the term “amplification” refers to any in vitro method for increasing the number of copies of a nucleic acid molecule with the use of one or more polypeptides having polymerase activity (e.g., one, two, three, four or more nucleic acid polymerases or reverse transcriptases). Nucleic acid amplification results in the incorporation of nucleotides into a DNA and/or RNA molecule or primer thereby forming a new nucleic acid molecule complementary to a template. The formed nucleic acid molecule and its template can be used as templates to synthesize additional nucleic acid molecules. As used herein, one amplification reaction may consist of many rounds of nucleic acid replication. DNA amplification reactions include, for example, polymerase chain reaction (PCR). One PCR reaction may consist of 5 to 100 cycles of denaturation and synthesis of a DNA molecule.

Nucleotide: As used herein, the term “nucleotide” refers to a base-sugar-phosphate combination. Nucleotides are monomeric units of a nucleic acid molecule (DNA and RNA). The term nucleotide includes ribonucleoside triphosphates ATP, UTP, CTG, GTP and deoxyribonucleoside triphosphates such as DATP, dCTP, dITP, dUTP, dGTP, dTTP, or derivatives thereof. Such derivatives include, for example, [α-S]dATP, 7-deaza-dGTP and 7-deaza-dATP. The term nucleotide as used herein also refers to dideoxyribonucleoside triphosphates (ddNTPs) and their derivatives. Illustrated examples of dideoxyribonucleoside triphosphates include, but are not limited to, ddATP, ddCTP, ddGTP, ddITP, and ddTTP. According to the present invention, a “nucleotide” may be unlabeled or detectably labeled by well known techniques. Detectable labels include, for example, radioactive isotopes, fluorescent labels, chemiluminescent labels, bioluminescent labels and enzyme labels.

Nucleic Acid Molecule: As used herein, the phrase “nucleic acid molecule” refers to a sequence of contiguous nucleotides (riboNTPs, dNTPs, ddNTPs, or combinations thereof) of any length. A nucleic acid molecule may encode a full-length polypeptide or a fragment of any length thereof, or may be non-coding. As used herein, the terms “nucleic acid molecule” and “polynucleotide” may be used interchangeably and include both RNA and DNA.

Oligonucleotide: As used herein, the term “oligonucleotide” refers to a synthetic or natural molecule comprising a covalently linked sequence of nucleotides that are joined by a phosphodiester bond between the 3′ position of the pentose of one nucleotide and the 5′ position of the pentose of the adjacent nucleotide.

Polypeptide: As used herein, the term “polypeptide” refers to a sequence of contiguous amino acids of any length. The terms “peptide,” “oligopeptide,” or “protein” may be used interchangeably herein with the term “polypeptide.”

Hybridization: As used herein, the terms “hybridization” and “hybridizing” refer to base pairing of two complementary single-stranded nucleic acid molecules (RNA and/or DNA) to give a double stranded molecule. As used herein, two nucleic acid molecules may hybridize, although the base pairing is not completely complementary.

Accordingly, mismatched bases do not prevent hybridization of two nucleic acid molecules provided that appropriate conditions, well known in the art, are used. In some aspects, hybridization is said to be under “stringent conditions.” By “stringent conditions,” as the phrase is used herein, is meant overnight incubation at 42° C. in a solution comprising: 50% formamide, 5×SSC (750 mM NaCl, 75m M trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at about 65° C.

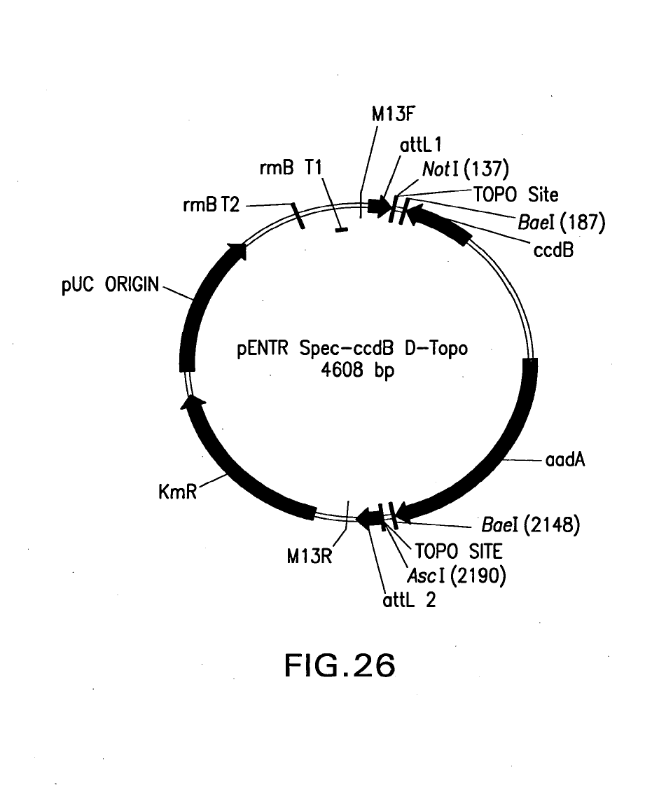

Derivative: As used herein the term “derivative”, when used in reference to a vector, means that the derivative vector contains one or more (e.g., one, two, three, four five, etc.) nucleic acid segments which share sequence similar to at least one vector represented in one or more of FIG. 1, 7, 8, 9, 13, 14, 15, 16, 17, 18, 19, 26, 30, 31, 32, 33, 34, 35, 36, 37, 41, 43, 44, 45, 46, 52, 53, 54, or 55. In particular embodiments, a derivative vector (1) may be obtained by alteration of a vector represented in FIG. 1, 7, 8, 9, 13, 14, 15, 16, 17, 18, 19, 26, 30, 31, 32, 33, 34, 35, 36, 37, 41, 43, 44, 45, 46, 52, 53, 54, or 55, or (2) may contain one or more elements (e.g., ampicillin resistance marker, attL1 recombination site, TOPO site, etc.) of a vector represented in FIG. 1, 7, 8, 9, 13, 14, 15, 16, 17, 18, 19, 26, 30, 31, 32, 33, 34, 35, 36, 37, 41, 43, 44, 45, 46, 52, 53, 54, or 55. Further, as noted above, a derivative vector may contain one or more element which shares sequence similarity (e.g., at least 50%, at least 60%, at least 70%, at least 80%, at least 90%, at least 95%, etc. sequence identity at the nucleotide level) to one or more element of a vector represented in FIG. 1, 7, 8, 9, 13, 14, 15, 16, 17, 18, 19, 26, 30, 31, 32, 33, 34, 35, 36, 37, 41, 43, 44, 45, 46, 52, 53, 54, or 55. Derivative vectors may also share at least at least 50%, at least 60%, at least 70%, at least 80%, at least 90%, at least 95%, etc. sequence identity at the nucleotide level to the complete nucleotide sequence of a vector represented in FIG. 1, 7, 8, 9, 13, 14, 15, 16, 17, 18, 19, 26, 30, 31, 32, 33, 34, 35, 36, 37, 41, 43, 44, 45, 46, 52, 53, 54, or 55. One example of a derivative vectors is the vector represented in FIG. 26 after the ccdB/spectinomycin resistance cassette has been replaced by another nucleic acid segment using a recombination reaction. Thus, derivative vectors include those which have been generated by performing a cloning reaction upon a vector represented in FIG. 1, 7, 8, 9, 13, 14, 15, 16, 17, 18, 19, 26, 30, 31, 32, 33, 34, 35, 36, 37, 41, 43, 44, 45, 46, 52, 53, 54, or 55. Derivative vectors also include vectors which have been generated by the insertion of elements of a vector represented in FIG. 1, 7, 8, 9, 13, 14, 15, 16, 17, 18, 19, 26, 30, 31, 32, 33, 34, 35, 36, 37, 41, 43, 44, 45, 46, 52, 53, 54, or 55 into another vector. Often these derivative vectors will contain at least 50%, at least 60%, at least 70%, at least 80%, at least 90%, at least 95%, etc. of the nucleic acid present in a vector represented in FIG. 1, 7, 8, 9, 13, 14, 15, 16, 17, 18, 19, 26, 30, 31, 32, 33, 34, 35, 36, 37, 41, 43, 44, 45, 46, 52, 53, 54, or 55. Derivative vectors also include progeny of any of the vectors referred to above, as well as vectors referred to above which have been subjected to mutagenesis (e.g., random mutagenesis).

Other terms used in the fields of recombinant nucleic acid technology and molecular and cell biology as used herein will be generally understood by one of ordinary skill in the applicable arts.

### Overview

The present invention relates to nucleic acid sequences encoding a polypeptide having a detectable activity, nucleic acid molecules comprising such sequences, and methods of joining nucleic acid molecules comprising such sequences to other nucleic acid molecules (which may comprise sequences encoding one or more polypeptides). The invention also relates to compositions comprising nucleic acid molecules of the invention, polypeptides (e.g., fusion polypeptides) encoded by such nucleic acid molecules, vectors comprising such nucleic acid molecules and derivatives thereof, and kits comprising such compositions.

### Polypeptides of the Invention

The invention also includes nucleic acid molecules that encode fusion proteins comprising the following three polypeptide portions: (1) a polypeptide encoded by a nucleic acid of interest (e.g., a nucleic acid segment which has been inserted into a vector), (2) a peptide or polypeptide encoded by all or part of cloning site (e.g., a restriction enzyme recognition site, a recombination site, a topoisomerase recognition site, etc.), and (3) a polypeptide having a detectable activity. The invention further includes fusion proteins which are encoded by such nucleic acid molecules, as well as (a) methods for making such nucleic acid molecules and fusions proteins and (b) compositions (e.g., reaction mixtures) comprising such nucleic acid molecules and fusions proteins.

The polypeptide portions referred to above may be connected in any order to form fusion proteins of the invention but typical orders included (1)-(2)-(3) and (3)-(2)-(1). In particular instances, a peptide or polypeptide encoded by all or part of cloning site may comprise one to three, three to five, five to eight, eight to ten, ten to fifteen, or fourteen to twenty amino acids.

As noted above, one component of fusion proteins of the invention may be encoded by a cloning site, such as a topoisomerase recognition site. Exemplary topoisomerase recognition sites comprise the sequences CCCTT and TCCTT. Topoisomerase recognition sequences are five nucleotides in length. Depending upon the reading frame of the polypeptides on either side of the topoisomerase site, it may be desirable to add one or two nucleotides on either side of the site and introduce either a di- or tri-peptide into the final fusion protein. For example, one nucleotide may be added at either end of the topoisomerase site, for example, so that the site with the additional nucleotide encodes a di-peptide. For the topoisomerase recognition sequence CCCTT, the codon duplexes thus generated are ACC CTT (encoding Thr-Leu), GCC CTT, (encoding Ala-Leu), TCC CTT, (encoding Ser-Leu), CCC CTT, (encoding Pro-Leu), CCC TTA, (encoding Pro-Leu), CCC TTG, (encoding Pro-Leu), CCC TTT, (encoding Pro-Phe), and CCC TTC, (encoding Pro-Phe). In many organisms, the dipeptides encoded by these codon duplexes would be Thr-Leu, Ser-Leu, Pro-Leu, Ala-Leu, Pro-Leu, and Pro-Phe. Thus, fusion proteins of the invention include those which comprise the following polypeptide portions: (1)-Thr-Leu-(3), (3)-Thr-Leu-(1), (1)-Ser-Leu-(3), (3)-Ser-Leu-(1), (1)-Pro-Leu-(3), (3)-Pro-Leu-(1), (1)-Ala-Leu-(3), (3)-Ala-Leu-(1), (1)-Pro-Leu-(3), (3)-Pro-Leu-(1), (1)-Pro-Phe-(3), and (3)-Pro-Phe-(1).

In some embodiments, it may be desirable to add two nucleotides on either side of a topoisomerase site so as to bring polypeptides encoded on the nucleic acid molecules to be joined into the same reading frame. This may result in the addition of a tri-peptide to the final fusion protein. For example, if the polypeptide encoded by the nucleic acid molecule on one side of the topoisomerase site is in the first reading frame and the polypeptide encoded by the nucleic acid molecule on the other side of the topoisomerase site is in the third reading frame, it may be desirable to add two nucleotides to either side of the topoisomerase site (or equivalently to either nucleic acid molecule) to bring the polypeptides into the same reading frame. For example, in the sequence ATG-CCCTT-XXATG, the first ATG represents a polypeptide in the first reading frame of a first nucleic acid molecule CCCTT represents the nucleotides of the topoisomerase site and XXATG represents the nucleic acid sequence encoding a polypeptide in the third reading frame on the second nucleic acid molecule. In order to bring the two polypeptides into the same reading frame (i.e., put the ATG codons in the same reading frame) two nucleotides must be added to either side of the topoisomerase site or one to each side. When two nucleotides are added, for example, on the 3′ side of the topoisomerase site, the nucleic acid sequence and first two amino acids would be as above (i.e., CCC TTA, (encoding Pro-Leu), CCC TTG, (encoding Pro-Leu), CCC TTT, (encoding Pro-Phe), and CCC TTC, (encoding Pro-Phe) and the third amino acid could be any of the twenty naturally occurring amino acids depending upon the nucleotides one the second nucleic acid molecule (i.e., XX) and the second of the two nucleotides added. If the two nucleotides added are N₁ and N₂ the final nucleic acid molecule would have the sequence ATG-CCC-TTN₁-N₂XX-ATG. Thus, the tri-peptide may have the sequence Pro-(Phe or Leu)-Xaa where Xaa represents any of the naturally occurring amino acids. In like fashion, one skilled in the art can readily determine the peptide sequences generated by adding two nucleotides to the 5′-side of the topoisomerase site, or by adding one nucleotide to either side of the topoisomerase site. Fusion proteins comprising such sequences are within the scope of the present invention.

One example of an amino acid sequence which may be encoded by a cloning site is the following: Pro-Ala-Phe-Leu-Tyr-Lys-Val-Gly-Ile-Ile-Arg-Lys-His-Cys-Leu-Ser-Ile-Cys-Cys-Asn-Glu-Gln-Val-Thr-Ile-Ser-Gln-Asn-Lys-Ile-Ile-Ile (SEQ ID NO:56). This amino acid sequence is encoded by one of the six reading frames of an attL2 recombination site. This amino acid sequence may be present in fusion proteins due to the fact that there are no stop codons present in the reading of the attL2 site which encodes this amino acid sequence. Thus, when a fusion protein of the order (1)-(2)-(3) or (3)-(2)-(1) contains an attL2 site as the cloning site (i.e., component (2)). The amino acid sequence referred to above will often be encoded by an attL2 recombination site. Further this amino acid sequence may only comprise part of the amino acid sequence encoded by a portion of an attL2 recombination site. Thus, in particular embodiments, proteins of the invention will contain at least two, three, four, five, six, seven, eight, nine, ten, eleven, twelve, thirteen, fourteen, fifteen, sixteen, seventeen, eighteen, nineteen, twenty, twenty-five, or thirty amino acids of the sequence Pro-Ala-Phe-Leu-Tyr-Lys-Val-Gly-Ile-Ile-Arg-Lys-His-Cys-Leu-Ser-Ile-Cys-Cys-Asn-Glu-Gln-Val-Thr-Ile-Ser-Gln-Asn-Lys-Ile-Ile-Ile (SEQ ID NO:57). The invention further includes fusion proteins which contain a full-length amino acid sequence encoded by any of the six reading frames of any of the recombination sites set out in Table 4, as well as sub-portions of such amino acid sequences of the lengths set out above for the attL2 recombination site.

Polypeptides having a detectable activity which may be included in fusion proteins of the invention include those which function as reporters. Examples of suitable reporters are β-lactamases. When export of the fusion protein from the cell is not desired, β-lactamase polypeptides which may be used in methods and compositions of the invention will typically not contain a functional signal peptide. This is so because signal peptides of some β-lactamase polypeptides have been found to function in both eukaryotic and prokaryotic cells. In contrast, when export of the fusion protein from the cell is desired, β-lactamase polypeptides which may be used in methods and compositions of the invention may contain a functional signal peptide. Further, in such instances, the β-lactamase polypeptide portion of the fusion protein may be located at the amino-terminus.

Polypeptides having a detectable activity which may be included in fusion proteins of the invention include those which function as detectable tags or affinity tags. Examples of such tags include peptides such as those which have affinity for molecules containing one or more arsenic atoms (e.g., FLASH™ peptides). Such tags include those which may contain one or more cysteines and are capable of specifically reacting with a biarsenical molecule. In many instances, these tag sequences contain four cysteines. These tags may contain, for example, the sequence Cys-Cys-X-Y-Cys-Cys, where X and Y are the same or different amino acids. Examples of such tags include the following:

| | |
|---|---|
| (1) Cys-Cys-Arg-Glu-Cys-Cys, | (SEQ ID NO:58) |
| (2) Cys-Cys-Pro-Gly-Gys-Cys, | (SEQ ID NO:59) |
| and | |
| (3) Ala-Gly-Gly-Cys-Cys-Pro-Gly-Cys- | (SEQ ID NO:60) |
| Cys-Gly-Gly-Gly. | |

The invention further includes peptides which are designed to bind to or binds to biarsenical compounds, as well as nucleic acid which encodes such peptides and proteins which contain such peptides. Such peptides, as well as biarsenical compounds themselves, are described in U.S. Pat. No. 6,451,569, the entire disclosure of which is incorporated herein by reference. One specific example of a peptide of the invention, which may be referred to as a tag, is Ala-Gly-Gly-Cys-Cys-Pro-Gly-Cys-Cys-Gly-Gly-Gly (SEQ ID NO:61). The invention thus includes peptides comprising this sequence, proteins which contain this sequence, and nucleic acids which encode this sequence.

Nucleic acids of the invention include those which have been adapted to encode tags but have been modified to have one or more particular activities or lack one or more particular activities. As an example, the nucleotide sequence shown in Table 10 was designed to encodes a tag which binds biarsenical compounds and to avoid hairpin loops, palindromes, dimer formation and the use of any rare tRNA codons. Thus, nucleic acids of the invention may be designed or selected such that they have particular properties both at the nucleic acid and amino acid level. For example, nucleic acids of the invention may be designed or selected such that they encode particular amino acid sequences but also have particular properties as nucleic acids either themselves or upon transcription. For example, such nucleic acid may be designed or selected such that they either contain particular restriction sites or that they lack sequences which are often recognized by restriction endonucleases (e.g., palindromes).

When nucleic acids of the invention are designed, codons may be selected to encode particular amino acids. These codons vary, to some extent, with the translation system of the organism used but one example of a codon usage chart is set out below in Table 1. Codon selection is one example of a way that nucleic acids of the invention (e.g., nucleic acids which encode particular tags such as a tetracysteine sequence) may be designed to have one or more desired properties (e.g., containing particular restriction sites, avoiding rare codons for a particular organism, etc.).

TABLE 1
Codon usage Chart

| | | | |
|---|---|---|---|
| TTT F Phe | TCT S Ser | TAT Y Tyr | TGT C Cys |
| TTC F Phe | TCC S Ser | TAC Y Tyr | TGC C Cys |
| TTA L Leu | TCA S Ser | TAA * Ter | TGA * Ter |
| TTG L Leu | TCG S Ser | TAG * Ter | TGG W Trp |
| CTT L Leu | CCT P Pro | CAT H His | CGT R Arg |
| CTC L Leu | CCC P Pro | CAC H His | CGC R Arg |
| CTA L Leu | CCA P Pro | CAA Q Gln | CGA R Arg |
| CTG L Leu | CCG P Pro | CAG Q Gln | CGG R Arg |
| ATT I Ile | ACT T Thr | AAT N Asn | AGT S Ser |
| ATC I Ile | ACC T Thr | AAC N Asn | AGC S Ser |
| ATA I Ile | ACA T Thr | AAA K Lys | AGA R Arg |
| ATG M Met | ACG T Thr | AAG K Lys | AGG R Arg |
| GTT V Val | GCT A Ala | GAT D Asp | GGT G Gly |
| GTC V Val | GCC A Ala | GAC D Asp | GGC G Gly |
| GTA V Val | GCA A Aia | GAA E Glu | GGA G Gly |
| GTG V Val | GCG A Ala | GAG E Glu | GGG G Gly |

For each triplet, the single and three letter abbreviation for the encoded amino acid is shown.
Stop codons are represented by *.

The invention thus includes variations of the nucleotide sequence GCT GGT GGC TGT TGT CCT GGC TGT TGC GGT GGC GGC (SEQ ID NO:62), set out in Table 10, but which encode the same amino acid sequence. Examples of such sequences include the following: (1) GCC GGC GGC TGT TGT CCT GGC TGT TGC GGT GGC GGC (SEQ ID NO:63), (2) GCT GGT GGC TGC TGC CCT GGC TGT TGC GGT GGC GGC (SEQ ID NO:64), (3) GCT GGT GGC TGT TGT CCT GGC TGT TGC GGT GGC GGC (SEQ ID NO:65), and (4) GCT GGT GGC TGT TGT CCA GGC TGT TGC GGT GGC GGC (SEQ ID NO:66), as well as sub-portions of these nucleotide sequences which encode the amino acid sequence Cys-Cys-X-X-Cys-Cys (e.g., Cys-Cys-Pro-Gly-Cys-Cys (SEQ ID NO:67)), wherein “X” is any amino acid. In particular embodiments, nucleic acid which encodes a tag of the invention will not contain a particular nucleotide (e.g., adenosine, guanine, thymine, or cytosine). As an example, several of the nucleotide sequence shown above do not contain any adenosines. Transcription products of such nucleic acids are less likely, for example, to form hairpins than transcription products which contain all four nucleotides commonly found in RNA.

The Xs in the tetracysteine sequence may be any amino acids and may be the same or different. Examples of dipeptides which may be positioned between the two sets of cysteine residues include the following: (1) Pro-Gly, (2) Gly-Gly, (3) Ala-Gly, (4) Gly-Pro, (5) Ser-Gly, (6) Pro-Pro, (7) Ala-Ser, (8) Ser-Ser, (9) Trp-Gly, (10) Pro-Trp, (11) Phe-Gly, etc.

Tag sequence of the invention include those which contain the sequence (N-terminus) Cys-Cys-X-X-Cys-Cys (C-terminus) but have one or more amino acids associated with (1) their N-terminus, (2) their C-terminus, or (3) both their N-terminus and C-terminus. These amino acid at either the N-terminus, the C-terminus, or both termini may be designed to confer one or more particular conformations (e.g., random coil, beta-sheet, alpha helix, etc.) upon the tetracysteine sequence, when the tag is present either alone or bound to another amino acid sequence (e.g., when the tag is one component of a fusion protein). Examples of peptides which may be located at either the N-terminus, the C-terminus, or both termini of the tag include the following: (1) Ala-Gly-Gly, (2) Gly-Ala-Gly, (3) Gly-Ala-Ala, (4) Ala-Ala-Gly, (5) Ala-Ala-Ala, (6) Ser-Gly-Gly, (7) Gly-Ser-Gly, (8) Gly-Gly-Ser, (9) Ser-Ser-Gly, (10) Gly-Gly-Gly-Gly, (11) Gly-Pro-Ser, (12) and Gly-Gly-Gly-Gly-Ser, etc.

The tag may be located at either the N-terminus or the C-terminus, or located internally. When internally located, the tag may be positioned between different portions of the same protein or may contain all of part of two different proteins at both the N-terminus and the C-terminus of the tag. In other words, an internally located tag may have the following primary amino acid structure:

Protein A1-Gly-Glv-Cys-Cys-Pro-Gly-Cys-Cys-Gly-Gly-Protein A2 (SEQ ID NO:68), with “Protein A1” being the N-terminus of a protein and “Protein A2” being the C-terminus of the same protein and with the underlined amino sequence being the tag. This tag need not be one which binds to biarsenical compounds and includes other tags described herein (e.g., polypeptides which have one or more activities associated with β-lactamases).

The invention further includes methods for detecting molecules (e.g., tagged proteins) bound to solid supports. Thus, in one aspect the invention includes contacting and/or binding a tagged molecule to a solid support and detecting that molecule on the solid support. The detection methods employed may be essentially non-quantitative, semi-quantitative, or quantitative. In other words, the detection methods employed may (1) merely indicate that the tagged molecule is present, (2) provide a basis for roughly estimating the amount of tagged molecule present, or (3) provide a reasonably good measure of the amount of tagged molecules present (e.g., +/−5%). These detection methods may be, for example, colorimetric or fluorescence based.

In particular embodiments, tagged polypeptides are bound to a solid support, after which the presence of the tag is detected. One example of a method of the invention involves connecting a first nucleic acid molecules with a second nucleic acid molecule, wherein (1) the first nucleic acid molecule (e.g., a vector) encodes a polypeptide tag (e.g., a polypeptide comprising the sequence Cys-Cys-X-X-Cys-Cys, referred to herein as a tetracysteine sequence) and the second nucleic acid molecule encodes another amino acid sequence and (2) the two nucleic acid molecules are connected such that the polypeptide tag and the other amino acid sequence are encoded in-frame as a fusion product. The fusion product is then expressed and contacted with a solid support, after which the presence of the tag is detected.

When tags and/or tagged proteins are detected on a solid support, the tags and/or tagged proteins may be contacted with one or more detection reagents prior to the time that the tag and/or tagged protein are contacted with the support or afterward. Using as an example the detection of a protein tagged with a peptide that binds one or more biarsenical compounds after the tagged protein has been subjected to gel electrophoresis and then contacted with a solid support which is in the form of a membrane (e.g., a PVDF membrane), the tagged protein may be contacted with the detection reagent(s) prior to the gel electrophoresis step, during gel electrophoresis (e.g., the detection reagent(s) may be in the gel), after gel electrophoresis is complete (e.g., while the tagged protein is in the gel but before the gel and/or tagged protein are contacted with the solid support), and/or after the tagged protein has been contacted with and/or binds to the solid support.

Solid supports which may be used in the practice of the invention include beads (e.g., silica gel, controlled pore glass, magnetic, Sephadex/Sepharose, cellulose), flat surfaces or chips (e.g., glass fiber filters, glass surfaces, metal surface (steel, gold, silver, aluminum, copper and silicon), capillaries, plastic (e.g., polyethylene, polypropylene, polyamide, polyvinylidenedifluoride membranes or microtiter plates); or pins or combs made from similar materials comprising beads or flat surfaces or beads placed into pits in flat surfaces such as wafers (e.g., silicon wafers). Examples of solid supports also include acrylic, styrene-methyl methacrylate copolymers, ethylene/acrylic acid, acrylonitrile-butadiene-styrene (ABS), ABS/polycarbonate, ABS/polysulfone, ABS/polyvinyl chloride, ethylene propylene, ethylene vinyl acetate (EVA), nitrocellulose, nylons (including nylon 6, nylon 6/6, nylon 6/6-6, nylon 6/9, nylon 6/10, nylon 6/12, nylon 11 and nylon 12), polycarylonitrile (PAN), polyacrylate, polycarbonate, polybutylene terephthalate (PBT), polyethylene terephthalate (PET), polyethylene (including low density, linear low density, high density, cross-linked and ultra-high molecular weight grades), polypropylene homopolymer, polypropylene copolymers, polystyrene (including general purpose and high impact grades), polytetrafluoroethylene (PTFE), fluorinated ethylene-propylene (FEP), ethylene-tetrafluoroethylene (ETFE), perfluoroalkoxyethylene (PFA), polyvinyl fluoride (PVA), polyvinylidene fluoride (PVDF), polychlorotrifluoroethylene (PCTFE), polyethylene-chlorotrifluoroethylene (ECTFE), polyvinyl alcohol (PVA), silicon styrene-acrylonitrile (SAN), styrene maleic anhydride (SMA), metal oxides, and glass.

Biarsenical compounds suitable for use with tetracysteine tags of the invention include FLASH™ and REASH™ compounds. FLASH™ and REASH™ Labeling reagents and kits may be obtained from Invitrogen Corp., Carlsbad, Calif. (see, e.g., cat. nos. P3050 and P3006). These reagents may be used in conjunction with proteins which contain a suitable C-C-X-X-C-C binding motif for a variety of applications. As examples, these materials may be used for in-gel detection of proteins and in vivo labeling (i.e., intracellular labeling). In vivo labeling may be used to determine the sub-cellular location of an expressed protein. For example, when the cell which is used for in vivo labeling is a eukaryotic cell, the locations of proteins which are present in such sub-cellular locations as the nucleolus, nucleus, endoplasmic reticulum, mitochondria, or cytoplasm may be determined.

FLASH™ and REASH™ biarsenical compounds, as well as other biarsenical compounds, may be complexed with dithiol EDT (1,2-ethanediol) which is believed to stabilize and solubilize biarsenical compounds.

When tetracysteine amino acid sequence having affinity for a biarsenical compound is employed in methods of the invention (e.g., to bind the protein which contain the tetracysteine amino acid sequence to a biarsenical compound), proteins which contains the tetracysteine amino acid sequence may be contacted with the biarsenical compound in the presence of a reducing agent. Exemplary reducing agents include dithiothreitol (DTT), beta-mercaptoethanol (BME), Tris(2-carboxyethyl) phosphine HCl (TCEP), 1,2-ethanedithiol (EDT), 2,3-dimercapto-1-propanesulfonic acid (DMPS), and meso-2,3-dimercaptosuccinic acid (DMSA), tri-n-butylphosphine (TBP), 2-mercaptoethanol (2-ME or β-ME), and mercaptoethanesulfonic acid (MES), and combinations thereof.

When a reducing reagent is included in compositions used to practice methods of the invention it may be present in any suitable concentration, for example, 0.1 mM, 0.5 mM, 0.75 mM, 1 mM, 1.5 mM, 2 mM, 3 mM, 5 mM, 7.5 mM, 10 mM, etc. Suitable reducing reagent concentrations for particular applications may be determined by performing methods of the invention without and reducing agents present, followed by analysis of results obtained. Suitable reducing reagent concentrations for particular applications may also be determined by performing methods of the invention with reducing reagents present at different concentrations, followed by analysis of results obtained.

Methods employing reducing reagents are set out in U.S. Application No. 60/515,575, filed Oct. 28, 2003, the entire disclosure of which is incorporated herein by reference. U.S. Application No. 60/515,575 is directed, in part to the reduction of spurious binding of biarsenical fluorophores to vicinal cysteines of endogenous proteins by using mono- and dithiols to compete with the binding reaction. In many instances, reagents used in methods described in this application will be employed such that the competition does not substantially hinder the desired binding of the fluorophore to a tetracysteine tag.

Methods of the invention include those which involve double labeling or dual labeling of cells with at least two biarsenical compounds (e.g., FLASH™ and REASH™). These methods allow one to follow the intracellular movements of proteins within cells. Along these lines, Gaietta, G., et al. (2002) Science 296:503-507 (the entire disclosure of which is incorporated herein by reference), describes the use of protein tagged with a tetracysteine domain to monitor the trafficking of proteins in cells.

When performing dual labeling methods, typically, the labels will be added at different times. For example, FLASH™ and REASH™ biarsenical compounds are used to label proteins in the same cell or cells, one of the compounds may be added at one time point and then the second compound may be added at a later time point. In this way, pools of proteins which contain an amino acid sequence that binds to the biarsenical compounds can be identified/distinguished. Such methods are disclosed, for example, in Gaietta, G., et al. (2002) Science 296:503-507, and may be used to study protein assembly, protein internalization and protein turnover.

When in vivo labeling of cells is employed, it will often be advantageous to add one or more compounds to the cell solution which absorb background light. One example of such a compound is Disperse Blue 3. The use of this compound in conjunction with in vivo labeling is discussed below in Example 9.

One example of a method which may be used to label cells which express a protein with a suitable tetracysteine motif with FLASH™-EDT2 is the following. Cells are labeled for 90 minutes at room temperature with 2.5 μM FLASH™-EDT2 in OptiMEM™ (Invitrogen Corp., CA, see, e.g., cat nos. 11058-021, 31985-062, 31985-070, 31985-088, 51985-034). Cells are then gently washed once with OptiMEM™ and visualized in OptiMEM™ containing 20 μM Disperse Blue (Sigma-Aldrich, cat. no. 215651). Cells may then be photographed using a fluoresceine (FITC) filter with excitation wavelength 460-490 nm and emission wavelength 515-550 nm.

One example of a method which may be used for in-gel detection of proteins which contain a suitable tetracysteine motif with FLASH™-EDT2 is the following. Total cell lysates (25-45 μg protein) may be FLASH™ labeled by incubation with 25-50 μM FLASH™-EDT2 in the presence of 1× Laemmli sample loading buffer (80 mM Tris pH 6.8, 3% SDS, 15% glycerol and 358 mM beta-mercaptoethanol). Samples are then heated to 100° C. for 3 minutes, cooled to room temperature and electrophoresed on a 4-20% Tris-glycine polyacrylamide gel (Invitrogen) at 200V. The gel is removed from its cassette, placed on a UV light box and visualized through an ethidium bromide filter. The gel is then Coomassie stained using SIMPLY BLUE™ SafeStain (Invitrogen).

In particular embodiments, tagged proteins will contain two tags. One of these tags may be used, for example, for immobilizing the protein and the other for detection. In one specific example proteins of the invention contain an affinity tag such as a tag which binds to a metal chelate affinity chromatography matrix (e.g., a 6 His sequence) and another tag (e.g., a tag which binds to a biarsenical compound). The tag which binds to a metal chelate affinity chromatography matrix may be used to immobilize the protein and the other tag may then be used for detection.

In many instances, solid supports will be of a type which will bind a tagged molecule through a process which is not specific for the tag itself. In other words, in many instances, the tag will be left free to react with reagents used for detection (e.g., biarsenical compounds, fluorescent substrates such as CCF2 or CCF4, etc.), when it is necessary to employ such reagents. One example of such a binding process is the attachment of a tagged protein to a nitrocellulose membrane.

In one aspect, tagged molecules are separated from other molecules in a mixture by gel electrophoresis, followed by transfer to a solid support, such as a membrane (e.g., a PVDF membrane or a nitrocellulose membrane). The tagged molecules (e.g., tagged proteins) are then exposed to an agent which renders them detectable (e.g., a biarsenical), if necessary, and then detected. In many instances, detection will occur while the tagged molecule remains associated with the membrane.

In another aspect, tagged molecules are applied to the solid support in admixture with others molecules. For example, when the tagged molecule is a protein, a cell extract or a mixture comprising in vitro transcription/translation system components, for examples, may be applied directly to a solid support. Detection of the tag may then be employed to determine whether the tagged protein is present and, if so, how much of the tagged protein is present. In such a situation, a solution containing the tagged protein may be spotted onto the solid support in a defined region. Solutions containing other samples and/or one or more standards may, optionally, be spotted at other locations on the same or a different solid support. The tagged protein in the solution may be quantified, for example, by comparing the amount of detectable signal to the detectable signal generated by at least one standard.

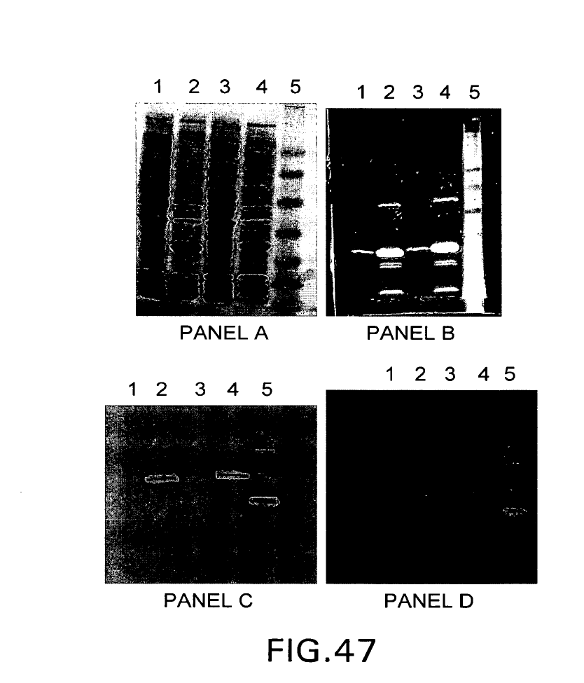

One example of a method described above is set out below in Example 8, which led to the results shown in FIG. 47. In this instance, proteins which contain a tetracysteine amino acid sequence (e.g., Cys-Cys-X-X-Cys-Cys, such as that present in the LUMIO™ tag described in Example 8) were produced using expression vectors. As used herein, the terms “Lumio” and “Flash” both refer to tetracysteine tags and are essentially the same. These tagged proteins were exposed to a biarsenical compound, separated from other molecules by gel electrophoresis, and then transferred to a PVDF membrane. When the PVDF membrane was exposed to UV light, tetracysteine tagged proteins were visible (FIG. 47D). Thus, methods of the invention further include those were the detection system employs the use of ultraviolet light. A number of biarsenical molecules which form fluorescent complexes when bound to tetracysteine tags are described in, for example, U.S. Pat. No. 6,451,569, the entire disclosure of which is incorporated herein by reference. Labeling kits employing a biarsenical compound for the detection of proteins which contain a tetracysteine amino acid sequence may be obtained, for example, from Invitrogen Corp., Carlsbad, Calif., cat. no. P3050.

The invention further includes nucleic acid molecules which encode fusion peptides which result from the connection of (1), (2), and/or (3), wherein (1) is a polypeptide encoded by a nucleic acid of interest, (2) is a peptide or polypeptide encoded by all or part of cloning site, and (3) a polypeptide having a detectable activity, as well as fusion proteins encoded by such nucleic acid molecules. Thus, the invention includes, for example, a fusion protein which contains one, two, three, four, five, six, seven, eight, nine, ten, etc. amino acid which are encoded for by (1), (2), and/or (3). As an example, the invention includes nucleic acid molecules wherein (2) is polypeptide or peptide encoded by a recombination sites (e.g., an attB 1 site, an attB2 site, etc.) and (3) is all or part of a β-lactamase polypeptide (e.g., a polypeptide with a β-lactamase activity, such as the ability to cleave a β-lactam ring). In such an instance, the fusion protein encoded by the nucleic acid molecule may comprise (1) one, two, three, four five, six, seven, eight, etc. amino acids encoded by an attB1 site and (2) all or part of a β-lactamase polypeptide. In particular instances, the amino acids of the fusion protein which are encoded by the attB1 site, or other recombination site, may be Pro-Ala-Phe-Leu-Tyr-Lys-Val-Val (SEQ ID NO:69), Ala-Phe-Leu-Tyr-Lys-Val-Val (SEQ ID NO:70), Phe-Leu-Tyr-Lys-Val-Val (SEQ ID NO:71), Leu-Tyr-Lys-Val-Val (SEQ ID NO:72), Tyr-Lys-Val-Val, Lys-Val-Val, Val-Val, Pro-Ala-Phe- or Val. Fusion proteins of the invention also include fusion proteins comprising an amino acid sequence encoded by any one of the recombination sites in Table 4 in any reading frame.

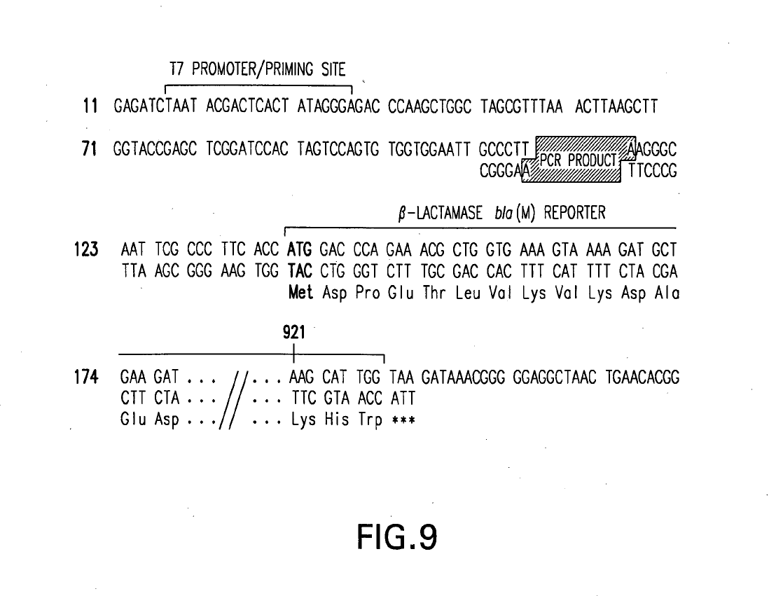

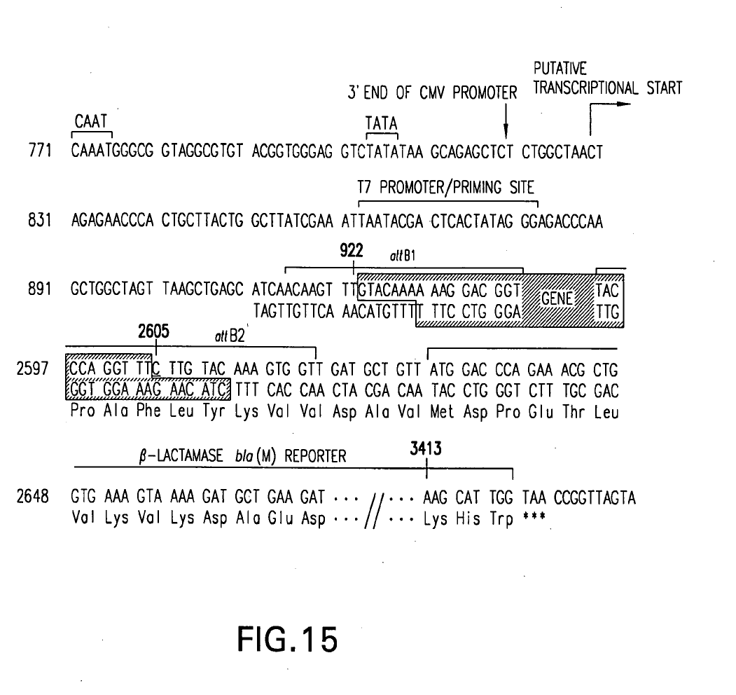

As noted above, the fusion protein may also comprise all or part of a β-lactamase. Furthermore, fusion proteins of the invention may comprise one, two, three, four, five, six, seven, eight, etc. amino acid encoded by a cloning sites (e.g., a recombination site) and all or part of the β-lactamase amino acid sequence shown in FIG. 9 or FIG. 15. Thus, fusion proteins of the invention include those which comprise the following amino acid sequences: (1) Pro-Ala-Phe-Leu-Tyr-Lys-Val-Val-X₀₋₂₀-Met-Asp-Pro-Glu-Thr-Leu-Val-Lys-Val-Lys-Asp-Ala-Glu-Asp (SEQ ID NO:73), (2) Val-Val-X₀₋₂₀-Met-Asp- (SEQ ID NO:74), (3) Lys-Val-Val-X₀₋₂₀-Met-Asp-Pro-Glu-Thr-Leu-Val-Lys-Val (SEQ ID NO:75), or (4) Val-Val-X₀₋₂₀-Met-Asp-Pro-Glu (SEQ ID NO:76), wherein X represents between 0 and 20 amino acids which may be the same or different.

In a specific embodiment of the invention, nucleic acid molecules of the invention may comprise a nucleic acid sequence encoding a polypeptide having an enzymatic activity (e.g., β-lactamase activity). In some embodiments, nucleic acid molecules of the invention may comprise nucleic acid sequence encoding a polypeptide having a detectable β-lactamase activity. Assays for β-lactamase activity are known in the art. U.S. Pat. Nos. 5,955,604, issued to Tsien, et al. Sep. 21, 1999, 5,741,657 issued to Tsien, et al., Apr. 21, 1998, 6,031,094, issued to Tsien, et al., Feb. 29, 2000, 6,291,162, issued to Tsien, et al., Sep. 18, 2001, and 6,472,205, issued to Tsien, et al. Oct. 29, 2002, disclose the use of β-lactamase as a reporter gene and fluorogenic substrates for use in detecting β-lactamase activity and are specifically incorporated herein by reference. In one embodiment of the invention, a nucleic acid sequence encoding a polypeptide having a detectable activity may be a nucleic acid sequence encoding a polypeptide having β-lactamase activity and desired host cells may be identified by assaying the host cells for β-lactamase activity.

A β-lactamase catalyzes the hydrolysis of a β-lactam ring. Those skilled in the art will appreciate that the sequences of a number of polypeptides having β-lactamase activity are known. In addition to the specific β-lactamases disclosed in the Tsien, et al. patents listed above, any polypeptide having β-lactamase activity is suitable for use in the present invention.

β-lactamases are classified based on amino acid and nucleotide sequence (Ambler, R. P., Phil. Trans. R. Soc. Lond. [Ser.B.] 289: 321-331 (1980)) into classes A-D. Class A β-lactamases possess a serine in the active site and have an approximate weight of 29 kd. This class contains the plasmid-mediated TEM β-lactamases such as the RTEM enzyme of pBR322. Class B β-lactamases have an active-site zinc bound to a cysteine residue. Class C enzymes have an active site serine and a molecular weight of approximately 39 kd, but have no amino acid homology to the class A enzymes. Class D enzymes also contain an active site serine. Representative examples of each class are provided below with the accession number at which the sequence of the enzyme may be obtained in the indicated database. The sequences of the enzymes in the following lists are specifically incorporated herein by reference.

| | Accession | |
|---|---|---|
| | No. | Data Bank |
| Class A β-lactamases | | |
| Bacteroides fragilis CS30 | L13472 | GenBank |
| Bacteroides uniformis WAL-7088 | P30898 | SWISS-PROT |
| PER-1, P. aeruginosa RNL-1 | P37321 | SWISS-PROT |
| Bacteroides vulgatus CLA341 | P30899 | SWISS-PROT |
| OHIO-1, Enterobacter cloacae | P18251 | SWISS-PROT |
| SHV-1, K. pneumoniae | P23982 | SWISS-PROT |
| LEN-1, K. pneumoniae LEN-1 | P05192 | SWISS-PROT |
| TEM-1, E. coli | P00810 | SWISS-PROT |
| Proteus mirabilis GN179 | P30897 | SWISS-PROT |
| PSE-4, P. aeruginosa Dalgleish | P16897 | SWISS-PROT |
| Rhodopseudomonas capsulatus SP108 | P14171 | SWISS-PROT |
| NMC, E. cloacae NOR-1 | P52663 | SWISS-PROT |
| Sme-1, Serratia marcescens S6 | P52682 | SWISS-PROT |
| OXY-2, Klebsiella oxytoca D488 | P23954 | SWISS-PROT |
| K. oxytoca E23004/SL781/SL7811 | P22391 | SWISS-PROT |
| S. typhimurium CAS-5 | X92507 | GenBank |
| MEN-1, E. coli MEN | P28585 | SWISS-PROT |
| Serratia fonticola CUV | P80545 | SWISS-PROT |
| Citrobacter diversus ULA27 | P22390 | SWISS-PROT |
| Proteus vulgaris 5E78-1 | P52664 | SWISS-PROT |
| Burkholderia cepacia 249 | U85041 | GenBank |
| Yersinia enterocolitica serotype O:3/Y-56 | Q01166 | SWISS-PROT |
| M. tuberculosis H37RV | Q10670 | SWISS-PROT |
| S. clavuligerus NRRL 3585 | Z54190 | GenBank |
| III, Bacillus cereus 569/H | P06548 | SWISS-PROT |
| B. licheniformis 749/C | P00808 | SWISS-PROT |
| I, Bacillus mycoides NI10R | P28018 | SWISS-PROT |
| I, B. cereus 569/H/9 | P00809 | SWISS-PROT |
| I, B. cereus 5/B | P10424 | SWISS-PROT |
| B. subtilis 168/6GM | P39824 | SWISS-PROT |
| 2, Streptomyces cacaoi DSM40057 | P14560 | SWISS-PROT |
| Streptomyces badius DSM40139 | P35391 | SWISS-PROT |
| Actinomadura sp. strain R39 | X53650 | GenBank |
| Nocardia lactamdurans LC411 | Q06316 | SWISS-PROT |
| S. cacaoi KCC S0352 | Q03680 | SWISS-PROT |
| ROB-1, H. influenzae F990/LNPB51/ | P33949 | SWISS-PROT |
| serotype A1 | | |
| Streptomyces fradiae DSM40063 | P35392 | SWISS-PROT |
| Streptomyces lavendulae DSM2014 | P35393 | SWISS-PROT |
| Streptomyces albus G | P14559 | SWISS-PROT |
| S. lavendulae KCCS0263 | D12693 | GenBank |
| Streptomyces aureofaciens | P10509 | SWISS-PROT |
| Streptomyces cellulosae KCCS0127 | Q06650 | SWISS-PROT |
| Mycobacterium fortuitum | L25634 | GenBank |
| S. aureus PC1/SK456/NCTC9789 | P00807 | SWISS-PROT |
| BRO-1, Moraxella catarrhalis ATCC | Z54181 | GenBank; |
| 53879 | Q59514 | SWISS-PROT |
| Class B β-lactamase | | |
| II, B. cereus 569/H | P04190 | SWISS-PROT |
| II, Bacillus sp. 170 | P10425 | SWISS-PROT |
| II, B. cereus 5/B/6 | P14488 | SWISS-PROT |
| Chryseobacterium meningosepticum | X96858 | GenBank |
| CCUG4310 | | |
| IMP-1, S. marcescens AK9373/TN9106 | P52699 | SWISS-PROT |
| B. fragilis TAL3636/TAL2480 | P25910 | SWISS-PROT |
| Aeromonas hydrophila AE036 | P26918 | SWISS-PROT |
| L1, Xanthomonas maltophilia IID 1275 | P52700 | SWISS-PROT |
| Class C β-lactamase | | |
| Citrobacter freundii OS60/GN346 | P05193 | SWISS-PROT |
| E. coli K-12/MG1655 | P00811 | SWISS-PROT |
| P99, E. cloacae P99/Q908R/MHN1 | P05364 | SWISS-PROT |
| Y. enterocolitica IP97/serotype O:5B | P45460 | SWISS-PROT |
| Morganella morganii SLM01 | Y10283 | GenBank |
| A. sobria 163a | X80277 | GenBank |
| FOX-3, K. oxytoca 1731 | Y11068 | GenBank |
| K. pneumoniae NU2936 | D13304 | GenBank |
| P. aeruginosa PAO1 | P24735 | SWISS-PROT |
| S. marcescens SR50 | P18539 | SWISS-PROT |
| Psychrobacter immobilis A5 | X83586 | GenBank |
| Class D β-lactamases | | |
| OXA-18, Pseudomonas aeruginosa Mus | U85514 | GenBank |
| OXA-9, Klebsiella pneumoniae | P22070 | SWISS-PROT |
| Aeromonas sobria AER 14 | X80276 | GenBank |
| OXA-1, Escherichia coli K10-35 | P13661 | SWISS-PROT |
| OXA-7, E. coli 7181 | P35695 | SWISS-PROT |
| OXA-11, P. aeruginosa ABD | Q06778 | SWISS-PROT |
| OXA-5, P. aeruginosa 76072601 | Q00982 | SWISS-PROT |
| LCR-1, P. aeruginosa 2293E | Q00983 | SWISS-PROT |
| OXA-2, Salmonella typhimurium type 1A | P05191 | SWISS-PROT |

Those skilled in the art will appreciate that any of the β-lactamase For additional β-lactamases and a more detailed description of substrate specificities, consult Bush et al. (1995) Antimicrob. Agents Chemother. 39:1211-1233. Those skilled in the art will appreciate that the polypeptides having β-lactamase activity disclosed herein may be altered by for example, mutating, deleting, and/or adding one or more amino acids and may still be used in the practice of the invention so long as the polypeptide retains detectable β-lactamase activity. An example of a suitably altered polypeptide having β-lactamase activity is one from which a signal peptide sequence has been deleted and/or altered such that the polypeptide is retained in the cytosol of prokaryotic and/or eukaryotic cells. The amino acid sequence of one such polypeptide is provided in Table 2.

TABLE 2
Amino acid sequence of a polypeptide having β-lactamase
activity (SEQ ID NO:77).

```
Met Gly His Pro Glu Thr Leu Val Lys Val Lys Asp Ala Gln Asp Gln
  1 5 10 15
Leu Gly Ala Arg Val Gly Tyr Ile GLu Leu Asp Leu Asn Ser Gly Lys
                        20 25 30
Ile Leu Glu Ser Phe Arg Pro Glu Glu Arg Phe Pro Met Met Ser Thr
                  35 40 45
Phe Lys Val Leu Len Cys Gly Ala Val Leu Ser Arg Asp Asp Ala Gly
          50 55 60
Gln Glu Gln Leu Gly Arg Arg Ile His Tyr Ser Gln Asn Asp Leu Val
65 70 75 80
Gln Tyr Ser Pro Val Thr Glu Lys His Leu Thr Asp Gly Met Thr Val
                                85 90 95
Arg Glu Leu Cys Ser Ala Ala Ile Thr Met Ser Asp Asn Thr Ala Ala
                        100 105 110
Asn Leu Len Leu Thr Thr Ile Gly Gly Pro Lys Glu Len Thr Ala Phe
                115 120 125
Leu His Asn Met Gly Asp His Val Thr Arg Len Asp His Trp Glu Pro
        130 135 140
Glu Len Asn Glu Ala Ile Pro Asn Asp Glu Arg Asp Thr Thr Met Pro
145 150 155 160
Val Ala Met Ala Thr Thr Leu Arg Lys Leu Leu Thr Gly Glu Leu Leu
                                165 170 175
Thr Leu Ala Ser Arg Gln Gln Leu Ile Asp Trp Met Glu Ala Asp Lys
                        180 185 190
Val Ala Gly Pro Len Len Arg Ser Ala Leu Pro Ala Gly Trp Phe Ile
                195 200 205
Ala Asp Lys Ser Gly Ala Gly Glu Arg Gly Ser Arg Gly Ile Ile Ala
        210 215 220
Ala Leu Gly Pro Asp Gly Lys Pro Ser Arg Ile Val Val Ile Tyr Thr
225 230 235 240
Thr Gly Ser Gln Ala Thr Met Asp Glu Arg Asn Arg Gln Ile Ala Glu
                                245 250 255
Ile Gly Ala Ser Leu Ile Lys His Trp
                        260 265
```

One skilled in the art will appreciate that the sequence in Table 2 may be modified and still be within the scope of the present invention. For example, with reference to FIG. 15, the Gly-His sequence of the polypeptide in Table 2 can be changed to an Asp without departing from the spirit of the invention.

As described in the above-referenced United States patents, host cells to be assayed may be contacted with a fluorogenic substrate for β-lactamase activity. In the presence of β-lactamase, the substrate is cleaved and the fluorescence emission spectrum of the substrate is altered. As an example, un-cleaved substrate may fluoresce green (i.e., have an emission maxima at approximately 520 nm) when excited with light having a wavelength of 405 nm and the cleaved substrate may fluoresce blue (i.e., have an emission maxima at approximately 447 nm). By determining the ratio of green fluorescence intensity to blue fluorescence intensity it is possible to determine the amount of β-lactamase produced and from that, to calculate what % of the cells express β-lactamase. Kits for conducting a fluorescence-based β-lactamase assay are commercially available, for example, from PanVera, LLC, Madison, Wis., catalog number K1032 now owned by Invitrogen Corporation, Carlsbad, Calif.

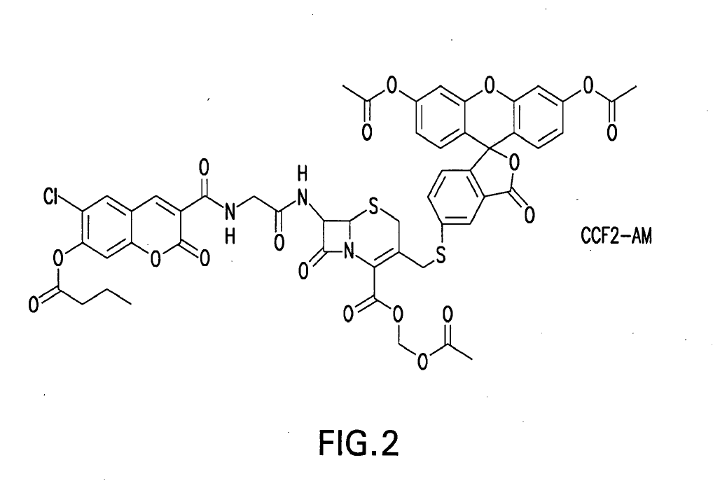

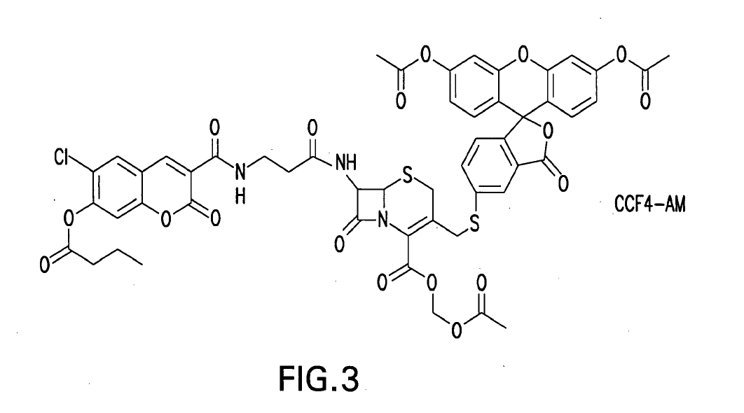

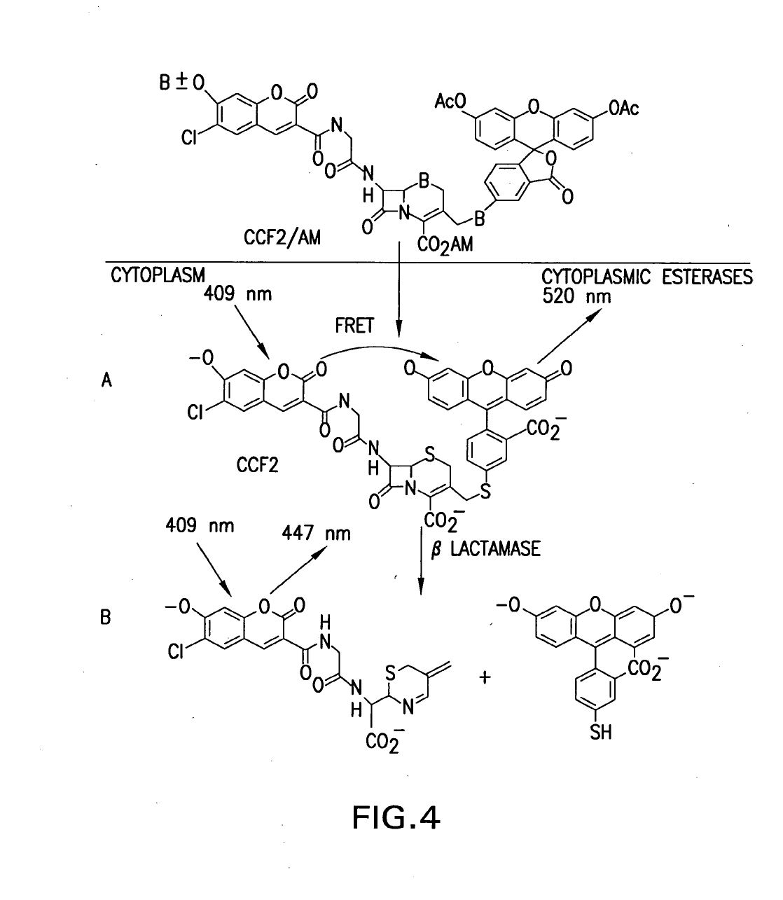

β-lactam fluorogenic substrates for use in the present invention include those which comprise a fluorescence donor moiety and a fluorescence acceptor moiety linked to a cephalosporin backbone such that, upon hydrolysis of the β-lactam, the acceptor moiety is released from the molecule. Before the β-lactam is hydrolyzed, the donor and acceptor moiety are positioned such that efficient fluorescence resonance energy transfer (FRET) occurs. Upon excitation with light of a suitable wavelength, fluorescence from the acceptor moiety is observed. After hydrolysis of the β-lactam, the acceptor moiety is released from the molecule and the FRET is disrupted resulting in a change in the fluorescence emission spectrum. An example of a suitable fluorescence donor molecule is a coumarin or derivative thereof (e.g., 6-chloro-7-hydroxycoumarin) and examples of suitable acceptor moieties include, but are not limited to, fluoresceine, rhodol, or rhodamine or derivatives thereof. Examples of suitable substrates include CCF2 and the acetoxymethyl ester derivative thereof (CCF2/AM) and CCF4 and the acetoxymethyl ester derivative thereof (CCF4/AM). Those skilled in the art will appreciate that the ester derivatives are membrane permeable and are de-esterified inside a cell by the action of endogenous esterase enzymes. The structures of CCF2 and CCF4 are provided in FIGS. 2 and 3 respectively. A schematic showing entry of the esterified substrate into a host cell, subsequent de-esterification and hydrolysis of CCF2 by a β-lactamase is shown in FIG. 4.

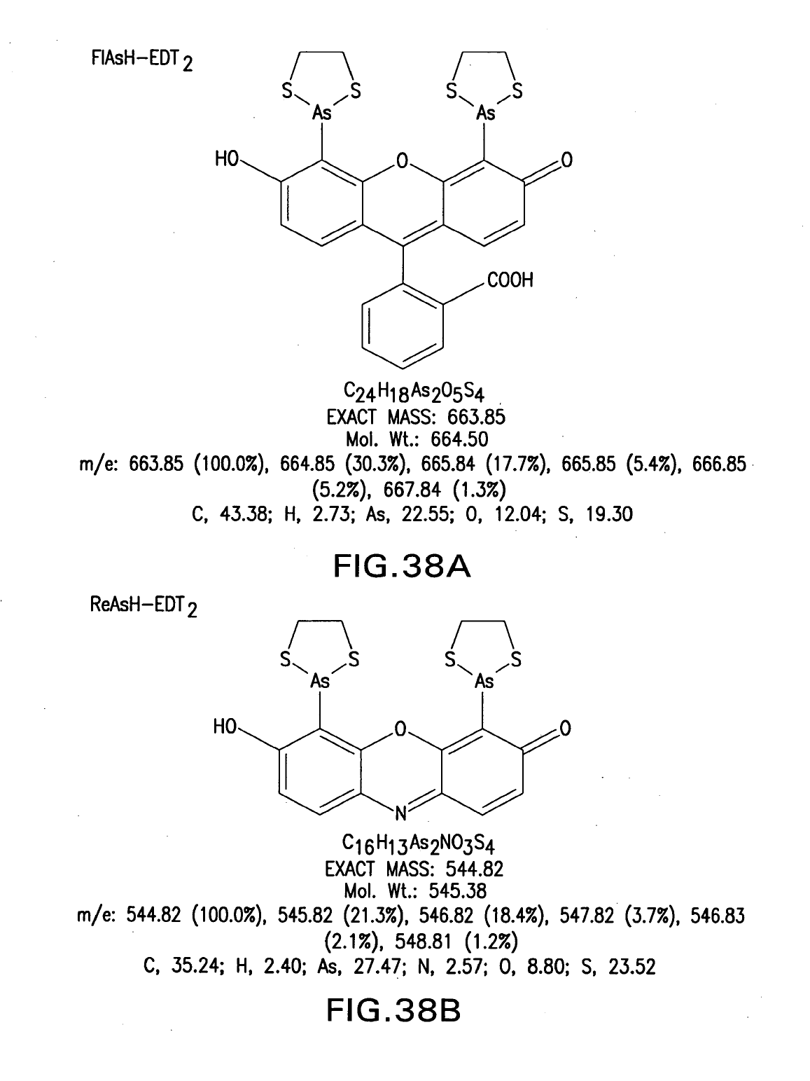

In some embodiments, nucleic acid molecules comprising a nucleic acid sequence encoding a polypeptide having a detectable activity may encode a polypeptide having the ability to bind to specific molecules or classes of molecules. In one embodiment, polypeptides having a detectable activity may have the ability to molecules comprising one or more arsenic atoms. One non-limiting example of a polypeptide having the ability to bind molecules comprising one or more arsenic atoms is -Ala-Gly-Gly-Cys-Cys-Pro-Gly-Cys-Cys-Gly-Gly-Gly-(SEQ ID NO:78). This polypeptide sequence may be placed at any position in a fusion protein comprising it, for example, at the N-terminus, at one or more internal positions, and/or at the C-terminus. The present invention also encompasses derivatives of this polypeptide, for example, one or more of the non-cysteine amino acids may be substituted. Polypeptides of this type may bind to molecules comprising one or more arsenic atoms (see, for example, U.S. Pat. Nos. 5,932,474, 6,008,378, 6,054,271, and 6,451,569 and published international patent application WO 01/53325A2). Upon binding of the polypeptides to the molecules comprising one or more arsenic atoms, the molecules may undergo a change in spectral properties (e.g., fluorescent properties). For example, upon binding of a polypeptides, the molecules comprising one or more arsenic atoms may become fluorescent. FIGS. 38A-38B provide structures of suitable molecules comprising one or more arsenic atoms for practice of this aspect of the invention.

### Recombination Sites

Recombination sites for use in the invention may be any nucleic acid that can serve as a substrate in a recombination reaction. Such recombination sites may be wild-type or naturally occurring recombination sites, or modified, variant, derivative, or mutant recombination sites. Examples of recombination sites for use in the invention include, but are not limited to, phage-lambda recombination sites (such as attP, attB, attL, and attR and mutants or derivatives thereof) and recombination sites from other bacteriophages such as phi80, P22, P2, 186, P4 and P1 (including lox sites such as loxP and loxP511).

Recombination proteins and mutant, modified, variant, or derivative recombination sites for use in the invention include those described in U.S. Pat. Nos. 5,888,732, 6,143,557, 6,171,861, 6,270,969, and 6,277,608 and in U.S. application Ser. No. 09/438,358, filed Nov. 12, 1999, which are specifically incorporated herein by reference. Mutated att sites (e.g., attB 1-10, attP 1-10, attR 1-10 and attL 1-10) are described in U.S. application Ser. No. 09/517,466, filed Mar. 2, 2000, and 09/732,914, filed Dec. 11, 2000 (published as US 2002/0007051-A1) the disclosures of which are specifically incorporated herein by reference in their entirety. Other suitable recombination sites and proteins are those associated with the GATEWAY® Cloning Technology systems available from Invitrogen Corporation, Carlsbad, Calif., and are described in the associated product literature, the entire disclosures of all of which are specifically incorporated herein by reference in their entireties.

Recombination sites that may be used in the present invention include att sites. The 15 bp core region of the wild-type att site (GCTTTTTTAT ACTAA (SEQ ID NO:79)), which is identical in all wild-type att sites, may be mutated in one or more positions. Engineered att sites that specifically recombine with other engineered att sites can be constructed by altering nucleotides in and near the 7 base pair overlap region, bases 6-12, of the core region. Thus, recombination sites suitable for use in the methods, molecules, compositions, and vectors of the invention include, but are not limited to, those with insertions, deletions or substitutions of one, two, three, four, or more nucleotide bases within the 15 base pair core region (see U.S. Pat. Nos. 5,888,732 and 6,277,608, which describe the core region in further detail, and the disclosures of which are incorporated herein by reference in their entireties). Recombination sites suitable for use in the methods, compositions, and vectors of the invention also include those with insertions, deletions or substitutions of one, two, three, four, or more nucleotide bases within the 15 base pair core region that are at least 50% identical, at least 55% identical, at least 60% identical, at least 65% identical, at least 70% identical, at least 75% identical, at least 80% identical, at least 85% identical, at least 90% identical, or at least 95% identical to this 15 base pair core region.

As a practical matter, whether any particular nucleic acid molecule is at least 50%, 60%, 70%, 75%, 80%, 85%, 90%, 95%, 96%, 97%, 98% or 99% identical to, for instance, a given recombination site nucleotide sequence or portion thereof can be determined conventionally using known computer programs such as DNAsis software (Hitachi Software, San Bruno, Calif.) for initial sequence alignment followed by ESEE version 3.0 DNA/protein sequence software (cabotetrog.mbb.sfu.ca) for multiple sequence alignments. Alternatively, such determinations may be accomplished using the BESTFIT program (Wisconsin Sequence Analysis Package, Genetics Computer Group, University Research Park, 575 Science Drive, Madison, Wis. 53711), which employs a local homology algorithm (Smith and Waterman, Advances in Applied Mathematics 2: 482-489 (1981)) to find the best segment of homology between two sequences. When using DNAsis, ESEE, BESTFIT or any other sequence alignment program to determine whether a particular sequence is, for instance, 95% identical to a reference sequence according to the present invention, the parameters are set such that the percentage of identity is calculated over the full length of the reference nucleotide sequence and that gaps in homology of up to 5% of the total number of nucleotides in the reference sequence are allowed. Computer programs such as those discussed above may also be used to determine percent identity and homology between two proteins at the amino acid level.

Analogously, the core regions in attB1, attP1, attL1 and attR1 are identical to one another, as arc the core regions in attB2, attP2, attL2 and attR2. Nucleic acid molecules suitable for use with the invention also include those comprising insertions, deletions or substitutions of one, two, three, four, or more nucleotides within the seven base pair overlap region (TTTATAC, bases 6-12 in the core region). The overlap region is defined by the cut sites for the integrase protein and is the region where strand exchange takes place. Examples of such mutants, fragments, variants and derivatives include, but are not limited to, nucleic acid molecules in which (1) the thymine at position 1 of the seven bp overlap region has been deleted or substituted with a guanine, cytosine, or adenine; (2) the thymine at position 2 of the seven bp overlap region has been deleted or substituted with a guanine, cytosine, or adenine; (3) the thymine at position 3 of the seven bp overlap region has been deleted or substituted with a guanine, cytosine, or adenine; (4) the adenine at position 4 of the seven bp overlap region has been deleted or substituted with a guanine, cytosine, or thymine; (5) the thymine at position 5 of the seven bp overlap region has been deleted or substituted with a guanine, cytosine, or adenine; (6) the adenine at position 6 of the seven bp overlap region has been deleted or substituted with a guanine, cytosine, or thymine; and (7) the cytosine at position 7 of the seven bp overlap region has been deleted or substituted with a guanine, thymine, or adenine; or any combination of one or more (e.g., two, three, four, five, etc.) such deletions and/or substitutions within this seven bp overlap region. The nucleotide sequences of representative seven base pair core regions are set out below.

Altered att sites have been constructed that demonstrate that (1) substitutions made within the first three positions of the seven base pair overlap (TTTATAC) strongly affect the specificity of recombination, (2) substitutions made in the last four positions (TTTATAC) only partially alter recombination specificity, and (3) nucleotide substitutions outside of the seven bp overlap, but elsewhere within the 15 base pair core region, do not affect specificity of recombination but do influence the efficiency of recombination. Thus, nucleic acid molecules and methods of the invention include those comprising or employing one, two, three, four, five, six, eight, ten, or more recombination sites which affect recombination specificity, particularly one or more (e.g., one, two, three, four, five, six, eight, ten, twenty, thirty, forty, fifty, etc.) different recombination sites that may correspond substantially to the seven base pair overlap within the 15 base pair core region, having one or more mutations that affect recombination specificity. Such molecules may comprise a consensus sequence such as NNNATAC wherein “N” refers to any nucleotide (i.e., may be A, G, T/U or C, or an analogue or derivative thereof). In particular embodiments, if one of the first three nucleotides in the consensus sequence is a T/U, then at least one of the other two of the first three nucleotides is not a T/U.

The core sequence of each att site (attB, attP, attL and attR) can be divided into functional units consisting of integrase binding sites, integrase cleavage sites and sequences that determine specificity. Specificity determinants are defined by the first three positions following the integrase top strand cleavage site. These three positions are shown with underlining in the following reference sequence: CAACTTTTTTATAC AAAGTTG (SEQ ID NO:80). Modification of these three positions (64 possible combinations) can be used to generate att sites that recombine with high specificity with other att sites having the same sequence for the first three nucleotides of the seven base pair overlap region. The possible combinations of first three nucleotides of the overlap region are shown in Table 3.

TABLE 3
Modifications of the First Three Nucleotides of
the att Site Seven Base Pair Overlap Region that
Alter Recombination Specificity.

| | | | |
|---|---|---|---|
| AAA | GAA | GAA | TAA |
| AAC | CAG | GAG | TAG |
| AAG | GAG | GAG | TAG |
| AAT | GAT | GAT | TAT |
| AGA | GGA | GGA | TGA |
| ACG | CCC | GGC | TGG |
| AGG | GGG | GGG | TGG |
| ACT | GGT | GGT | TCT |
| AGA | GGA | GGA | TGA |
| AGG | CGG | GGG | TGC |
| AGG | GGG | GGG | TGG |
| AGT | CGT | GGT | TGT |
| ATA | GTA | GTA | TTA |
| ATC | CTC | GTC | TTC |
| ATG | GTG | GTG | TTG |
| ATT | CTT | GTT | TTT |

Representative examples of seven base pair att site overlap regions suitable for use in methods, compositions and vectors of the invention are shown in Table 4. The invention further includes nucleic acid molecules comprising one or more (e.g., one, two, three, four, five, six, eight, ten, twenty, thirty, forty, fifty, etc.) nucleotides sequences set out in Table 2. Thus, for example, in one aspect, the invention provides nucleic acid molecules comprising the nucleotide sequence GAAATAC, GATATAC, ACAATAC, or TGCATAC.

TABLE 4
Representative Examples of Seven Base Pair att
Site Overlap Regions Suitable for use in the
recombination sites of the Invention.

| | | | |
|---|---|---|---|
| AAAATAC | CAAATAC | GAAATAC | TAAATAC |
| AACATAC | CACATAC | GACATAC | TACATAC |
| AAGATAC | CAGATAC | GAGATAC | TAGATAC |
| AATATAC | CATATAC | GATATAC | TATATAC |
| ACAATAC | CCAATAC | GCAATAC | TCAATAC |
| ACCATAC | CCCATAC | GCCATAC | TCCATAC |
| ACGATAC | CCGATAC | GCGATAC | TCGATAC |
| ACTATAC | CCTATAC | GCTATAC | TCTATAC |
| AGAATAC | CGAATAC | GGAATAC | TGAATAC |
| AGGATAC | CGCATAC | GGCATAC | TGCATAC |
| AGGATAC | CGGATAC | GGGATAC | TGGATAC |
| AGTATAC | CGTATAC | GGTATAC | TGTATAC |
| ATAATAC | CTAATAC | GTAATAC | TTAATAC |
| ATCATAC | CTCATAC | GTCATAC | TTCATAC |
| ATGATAC | CTGATAC | GTGATAC | TTGATAC |
| ATTATAC | CTTATAC | GTTATAC | TTTATAC |

As noted above, alterations of nucleotides located 3′ to the three base pair region discussed above can also affect recombination specificity. For example, alterations within the last four positions of the seven base pair overlap can also affect recombination specificity.

For example, mutated att sites that may be used in the practice of the present invention include attB1 (AGCCTGCTTT TTTGTACAAA CTTGT (SEQ ID NO:81)), attP1 (TACAGGTCAC TAATACCATC TAAGTAGTTG ATTCATAGTG ACTGGATATG TTGTGTTTTA CAGTATTATG TAGTCTGTTT TTTATGCAAA ATCTAATTTA ATATATTGAT ATTTATATCA TTTTACGTTT CTCGTTCAGC TTTTTTGTAC AAAGTTGGCA TTATAAAAAA GCATTGCTCA TCAATTTGTT GCAACGAACA GGTCACTATC AGTCAAAATA AAATCATTAT TTG (SEQ ID NO:82)), attL1 (CAAATAATGA TTTTATTTTG ACTGATAGTG ACCTGTTCGT TGCAACAAAT TGATAAGCAA TGCTTTTTTA TAATGCCAAC TTTGTACAAA AAAGCAGGCT (SEQ ID NO:83)), and attR1 (ACAAGTTTGT ACAAAAAAGC TGAACGAGAA ACGTAAAATG ATATAAATAT CAATATATTA AATTAGATTT TGCATAAAAA ACAGACTACA TAATACTGTA AAACACAACA TATCCAGTCA CTATG (SEQ ID NO:84)). Table 5 provides the sequences of the regions surrounding the core region for the wild type att sites (attB0, P0, R0, and L0) as well as a variety of other suitable recombination sites. Those skilled in the art will appreciated that the remainder of the site may be the same as the corresponding site (B, P, L, or R) listed above.

TABLE 5
Nucleotide sequences of att sites.

| | |
|---|---|
| attB0 | AGCCTGCTTT TTTATACTAA CTTGAGC |
| | (SEQ ID NO:85) |
| attP0 | GTTCAGCTTT TTTATACTAA GTTGGCA |
| | (SEQ ID NO:86) |
| attL0 | AGCCTGCTTT TTTATACTAA GTTGGCA |
| | (SEQ ID NO:87) |
| attR0 | GTTCAGCTTT TTTATACTAA CTTGAGC |
| | (SEQ ID NO:88) |
| attB1 | AGCCTGCTTT TTTGTACAAA CTTGT |
| | (SEQ ID NO:89) |
| attP1 | GTTCAGCTTT TTTGTACAAA GTTGGCA |
| | (SEQ ID NO:90) |
| attL1 | AGCCTGCTTT TTTGTACAAA GTTGGCA |
| | (SEQ ID NO:91) |
| attR1 | GTTCAGCTTT TTTGTACAAA CTTGT |
| | (SEQ ID NO:92) |
| attB2 | ACCCAGCTTT CTTGTACAAA GTGGT |
| | (SEQ ID NO:93) |
| attP2 | GTTCAGCTTT CTTGTACAAA GTTGGCA |
| | (SEQ ID NO:94) |
| attL2 | ACCCAGCTTT CTTGTACAAA GTTGGCA |
| | (SEQ ID NO:95) |
| attR2 | GTTCAGCTTT CTTGTACAAA GTGGT |
| | (SEQ ID NO:96) |
| attB5 | CAACTTTATT ATACAAAGTT GT |
| | (SEQ ID NO:97) |
| attP5 | GTTCAACTTT ATTATACAAA GTTGGCA |
| | (SEQ ID NO:98) |
| attL5 | CAACTTTATT ATACAAAGTT GGCA |
| | (SEQ ID NO:99) |
| attR5 | GTTCAACTTT ATTATACAAA GTTGT |
| | (SEQ ID NO:100) |
| attB11 | CAACTTTTCT ATACAAAGTT GT |
| | (SEQ ID NO:101) |
| attP11 | GTTCAACTTT TCTATACAAA GTTGGCA |
| | (SEQ ID NO:102) |
| attL11 | CAACTTTTCT ATACAAAGTT GGCA |
| | (SEQ ID NO:103) |
| attR11 | GTTCAACTTT TCTATACAAA GTTGT |
| | (SEQ ID NO:104) |
| attB17 | CAACTTTTGT ATACAAAGTT GT |
| | (SEQ ID NO:105) |
| attP17 | GTTCAACTTT TGTATACAAA GTTGGCA |
| | (SEQ ID NO:106) |
| attL17 | CAACTTTTGT ATACAAAGTT GGCA |
| | (SEQ ID NO:107) |
| attR17 | GTTCAACTTT TGTATACAAA GTTGT |
| | (SEQ ID NO:108) |
| attB19 | CAACTTTTTC GTACAAAGTT GT |
| | (SEQ ID NO:109) |
| attP19 | GTTCAACTTT TTCGTACAAA GTTGGCA |
| | (SEQ ID NO:110) |
| attL19 | CAACTTTTTC GTACAAAGTT GGCA |
| | (SEQ ID NO:111) |
| attR19 | GTTCAACTTT TTCGTACAAA GTTGT |
| | (SEQ ID NO:112) |
| attB20 | CAACTTTTTG GTACAAAGTT GT |
| | (SEQ ID NO:113) |
| attP20 | GTTCAACTTT TTGGTACAAA GTTGGCA |
| | (SEQ ID NO:114) |
| attL20 | CAACTTTTTG GTACAAAGTT GGCA |
| | (SEQ ID NO:115) |
| attR20 | GTTCAACTTT TTGGTACAAA GTTGT |
| | (SEQ ID NO:116) |
| attB21 | CAACTTTTTA ATACAAAGTT GT |
| | (SEQ ID NO:117) |
| attP21 | GTTCAACTTT TTAATACAAA GTTGGCA |
| | (SEQ ID NO:118) |
| attL21 | CAACTTTTTA ATACAAAGTT GGCA |
| | (SEQ ID NO:119) |
| attR21 | GTTCAACTTT TTAATACAAA GTTGT |
| | (SEQ ID NO:120) |

Other recombination sites having unique specificity (i.e., a first site will recombine with its corresponding site and will not substantially recombine with a second site having a different specificity) are known to those skilled in the art and may be used to practice the present invention. Corresponding recombination proteins for these systems may be used in accordance with the invention with the indicated recombination sites. Other systems providing recombination sites and recombination proteins for use in the invention include the FLP/FRT system from Saccharomyces cerevisiae, the resolvase family (e.g., γδ, TndX, TnpX, Tn3 resolvase, Hin, Hjc, Gin, SpCCE1, ParA, and Cin), and IS231 and other Bacillus thuringiensis transposable elements. Other suitable recombination systems for use in the present invention include the XerC and XerD recombinases and the psi, dif and cer recombination sites in E. coli. Other suitable recombination sites may be found in U.S. Pat. No. 5,851,808 issued to Elledge and Liu which is specifically incorporated herein by reference.

### Recombination Reactions

Those skilled in the art can readily optimize the conditions for conducting the recombination reactions described herein without the use of undue experimentation, based on the guidance provided herein and available in the art (see, e.g., U.S. Pat. Nos. 5,888,732 and 6,277,608, which are specifically incorporated herein by reference in their entireties). In a typical reaction from, about 50 ng to about 1000 ng of a second nucleic acid molecule may be contacted with a first nucleic acid molecule under suitable reaction conditions. Each nucleic acid molecule may be present in a molar ratio of from about 25:1 to about 1:25 first nucleic acid molecule:second nucleic acid molecule. In some embodiments, a first nucleic acid molecule may be present at a molar ratio of from about 10:1 to 1:10 first nucleic acid molecule:second nucleic acid molecule. In one embodiment, each nucleic acid molecule may be present at a molar ratio of about 1:1 first nucleic acid molecule:second nucleic acid molecule.

Typically, the nucleic acid molecules may be dissolved in an aqueous buffer and added to the reaction mixture. One suitable set of conditions is 4 μl CLONASE™ enzyme mixture (e.g., Invitrogen Corporation, Cat. Nos. 11791-019 and 11789-013), 4 μl 5× reaction buffer and nucleic acid and water to a final volume of 20 μl. This will typically result in the inclusion of about 200 ng of Int and about 80 ng of IHF in a 20 μl BP reaction and about 150 ng Int, about 25 ng IHF and about 30 ng Xis in a 20 μl LR reaction.

Proteins for conducting an LR reaction may be stored in a suitable buffer, for example, LR Storage Buffer, which may comprise about 50 mM Tris at about pH 7.5, about 50 mM NaCl, about 0.25 mM EDTA, about 2.5 mM Spermidine, and about 0.2 mg/ml BSA. When stored, proteins for an LR reaction may be stored at a concentration of about 37.5 ng/μl INT, 10 ng/μl IHF and 15 ng/μl XIS. Proteins for conducting a BP reaction may be stored in a suitable buffer, for example, BP Storage Buffer, which may comprise about 25 mM Tris at about pH 7.5, about 22 mM NaCl, about 5 mM EDTA, about 5 mM Spermidine, about 1 mg/ml BSA, and about 0.0025% Triton X-100. When stored, proteins for an BP reaction may be stored at a concentration of about 37.5 ng/μl INT and 20 ng/μl IHF. One skilled in the art will recognize that enzymatic activity may vary in different preparations of enzymes. The amounts suggested above may be modified to adjust for the amount of activity in any specific preparation of enzymes.

A suitable 5× reaction buffer for conducting recombination reactions may comprise 100 mM Tris pH 7.5, 88 mM NaCl, 20 mM EDTA, 20 mM Spermidine, and 4 mg/ml BSA. Thus, in a recombination reaction, the final buffer concentrations may be 20 mM Tris pH 7.5, 17.6 mM NaCl, 4 mM EDTA, 4 mM Spermidine, and 0.8 mg/ml BSA. Those skilled in the art will appreciate that the final reaction mixture may incorporate additional components added with the reagents used to prepare the mixture, for example, a BP reaction may include 0.005% Triton X-100 incorporated from the BP CLONASE™.

In some embodiments, particularly those in which attL sites are to be recombined with attR sites, the final reaction mixture may include about 50 mM Tris HCl, pH 7.5, about 1 mM EDTA, about 1 mg/ml BSA, about 75 mM NaCl and about 7.5 mM spermidine in addition to recombination enzymes and the nucleic acids to be combined.

In other embodiments, particularly those in which an attB site is to be recombined with an attP site, the final reaction mixture may include about 25 mM Tris HCl, pH 7.5, about 5 mM EDTA, about 1 mg/ml bovine serum albumin (BSA), about 22 mM NaCl, and about 5 mM spermidine.

In some embodiments, particularly those in which attL sites are to be recombined with attR sites, the final reaction mixture may include about 40 mM Tris HCl, pH 7.5, about 1 mM EDTA, about 1 mg/ml BSA, about 64 mM NaCl and about 8 mM spermidine in addition to recombination enzymes and the nucleic acids to be combined. One of skill in the art will appreciate that the reaction conditions may be varied somewhat without departing from the invention. For example, the pH of the reaction may be varied from about 7.0 to about 8.0; the concentration of buffer may be varied from about 25 mM to about 100 mM; the concentration of EDTA may be varied from about 0.5 mM to about 2 mM; the concentration of NaCl may be varied from about 25 mM to about 150 mM; and the concentration of BSA may be varied from 0.5 mg/ml to about 5 mg/ml. In other embodiments, particularly those in which an attB site is to be recombined with an attP site, the final reaction mixture may include about 25 mM Tris HCl, pH 7.5, about 5 mM EDTA, about 1 mg/ml bovine serum albumin (BSA), about 22 mM NaCl, about 5 mM spermidine and about 0.005% detergent (e.g., Triton X-100).

### Topoisomerase Cloning

The present invention also relates to methods of using one or more topoisomerases to generate a recombinant nucleic acid molecules of the invention (e.g., molecules comprising one or more nucleic acid sequence encoding a polypeptide having a detectable activity) comprising two or more nucleotide sequences, any one or more of which may comprise, for example, all or a portion of a nucleic acid sequence encoding a polypeptide having a detectable activity. Topoisomerases may be used in combination with recombinational cloning techniques described above. For example, a topoisomerase-mediated reaction may be used to attach one or more recombination sites to one or more nucleic acid segments. The segments may then be further manipulated and combined using, for example, recombinational cloning techniques.

In one aspect, the present invention provides methods for linking a first and at least a second nucleic acid segment (either or both of which may contain one or more nucleic acid sequences encoding a polypeptide having a detectable activity and/or sequences of interest) with at least one (e.g., 1, 2, 3, 4, 5, 6, 7, 8, 9, 10, etc.) topoisomerase (e.g., a type IA, type IB, and/or type II topoisomerase) such that either one or both strands of the linked segments are covalently joined at the site where the segments are linked.

A method for generating a double stranded recombinant nucleic acid molecule covalently linked in one strand can be performed by contacting a first nucleic acid molecule which has a site-specific topoisomerase recognition site (e.g., a type IA or a type II topoisomerase recognition site), or a cleavage product thereof, at a 5′ or 3′ terminus, with a second (or other) nucleic acid molecule, and optionally, a topoisomerase (e.g., a type IA, type IIB, and/or type II topoisomerase), such that the second nucleotide sequence can be covalently attached to the first nucleotide sequence. As disclosed herein, the methods of the invention can be performed using any number of nucleotide sequences, typically nucleic acid molecules wherein at least one of the nucleotide sequences has a site-specific topoisomerase recognition site (e.g., a type IA, type IB or type II topoisomerase), or cleavage product thereof, at one or both 5′ and/or 3′ termini.

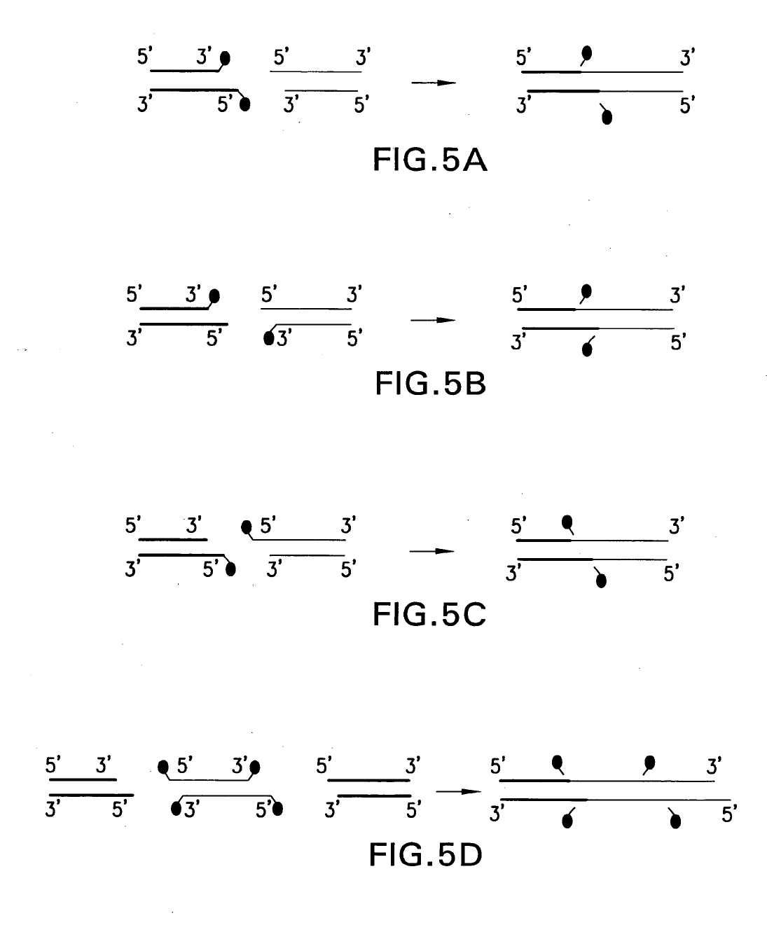

In some embodiments, two double-stranded nucleic acid molecules can be joined into a one larger molecule such that each strand of the larger molecule is covalently joined (e.g., the larger molecule has no nicks). With reference to FIG. 5, a first double-stranded nucleic acid molecule having a topoisomerase linked to each of the 5′ terminus and 3′ terminus of one end may be contacted with a second nucleic acid under conditions causing the linkage of both strands of the first nucleic acid molecule to both strands of the second nucleic acid molecule (FIG. 5A). The end of the first nucleic acid molecules to which the topoisomerases are attached may have either a 5′-overhang, 3′-overhang or be blunt ended. The end of the second nucleic acid molecule to be joined to the first nucleic acid molecule may have the same type of end as the topoisomerase-linked end of the first nucleic acid molecule. The end of the second molecule that is not to be joined may have a different end if directional joining of the segments is desired and may have the same type of end if directionality is not required.

In another embodiment, a first nucleic acid molecule having a topoisomerase bound to the 3′ terminus of one end, and a second nucleic acid molecule having a topoisomerase bound to the 3′ terminus of one end may be joined using the methods of the invention (FIG. 5B). A covalently linked double-stranded recombinant nucleic acid molecule is generated by contacting the ends containing the topoisomerase-charged substrate nucleic acid molecules.

FIG. 5C shows a first nucleic acid molecule having a topoisomerase bound to the 5′ terminus of one end, and a second nucleic acid molecule having a topoisomerase bound to the 5′ terminus of one end, and further shows the production of a covalently linked double-stranded recombinant nucleic acid molecule generated by contacting the ends containing the topoisomerase-charged substrate nucleic acid molecules.

FIG. 5D shows a nucleic acid molecule having a topoisomerase linked to each of the 5′ terminus and 3′ terminus of both ends, and further shows linkage of the topoisomerase-charged nucleic acid molecule to two nucleic acid molecules, one at each end. The topoisomerases at each of the 5′ termini and/or at each of the 3′ termini can be the same or different. Those skilled in the art will appreciate that nicked molecules (e.g., covalently joined in only one strand) may be produced by omitting one of the topoisomerases from the any one of the methods described above for FIGS. 5A-5D.

A method for generating a double stranded recombinant nucleic acid molecule covalently linked in both strands can be performed, for example, by contacting a first nucleic acid molecule having a first end and a second end, wherein, at the first end or second end or both ends, the first nucleic acid molecule has a topoisomerase recognition site (or cleavage product thereof) at or near the 5′ or 3′ terminus; at least a second nucleic acid molecule having a first end and a second end, wherein, at the first end or second end or both ends, the at least second double stranded nucleotide sequence has a topoisomerase recognition site (or cleavage product thereof) at or near a 5′ or 3′ terminus; and at least one site specific topoisomerase (e.g., a type IA and/or a type IB topoisomerase), under conditions such that all components are in contact and the topoisomerase can effect its activity. A covalently linked double stranded recombinant nucleic acid generated according to a method of this aspect of the invention is characterized, in part, in that it does not contain a nick in either strand at the position where the nucleic acid molecules are joined. In one embodiment, the method is performed by contacting a first nucleic acid molecule and a second (or other) nucleic acid molecule, each of which has a topoisomerase recognition site in addition to viral sequences an/or sequences of interest, or a cleavage product thereof, at the 3′ termini or at the 5′ termini of two ends to be covalently linked. In another embodiment, the method is performed by contacting a first nucleic acid molecule having a topoisomerase recognition site, or cleavage product thereof, at the 5′ terminus and the 3′ terminus of at least one end, and a second (or other) nucleic acid molecule having a 3′ hydroxyl group and a 5′ hydroxyl group at the end to be linked to the end of the first nucleic acid molecule containing the recognition sites. As disclosed herein, the methods can be performed using any number of nucleic acid molecules having various combinations of termini and ends.

Topoisomerases are categorized as type I, including type IA and type IB topoisomerases, which cleave a single strand of a double stranded nucleic acid molecule, and type II topoisomerases (gyrases), which cleave both strands of a nucleic acid molecule. Type IA and IB topoisomerases cleave one strand of a nucleic acid molecule. Cleavage of a nucleic acid molecule by type IA topoisomerases generates a 5′ phosphate and a 3′ hydroxyl at the cleavage site, with the type IA topoisomerase covalently binding to the 5′ terminus of a cleaved strand. In comparison, cleavage of a nucleic acid molecule by type IB topoisomerases generates a 3′ phosphate and a 5′ hydroxyl at the cleavage site, with the type TB topoisomerase covalently binding to the 3′ terminus of a cleaved strand. As disclosed herein, type I and type II topoisomerases, as well as catalytic domains and mutant forms thereof, are useful for generating double stranded recombinant nucleic acid molecules covalently linked in both strands according to a method of the invention.

Type IA topoisomerases include E. coli topoisomerase I, E. coli topoisomerase III, eukaryotic topoisomerase II, archeal reverse gyrase, yeast topoisomerase III, Drosophila topoisomerase III, human topoisomerase III, Streptococcus pneumoniae topoisomerase III, and the like, including other type IA topoisomerases (see Berger, Biochim. Biophys. Acta 1400:3-18, 1998; DiGate and Marians, J. Biol. Chem. 264:17924-17930, 1989; Kim and Wang, J. Biol. Chem. 267:17178-17185, 1992; Wilson, et al., J. Biol. Chem. 275:1533-1540, 2000; Hanai, et al., Proc. Natl. Acad. Sci., USA 93:3653-3657, 1996, U.S. Pat. No. 6,277,620, each of which is incorporated herein by reference). E. coli topoisomerase III, which is a type IA topoisomerase that recognizes, binds to and cleaves the sequence 5′-GCAACTT-3′, can be particularly useful in a method of the invention (Zhang, et al., J. Biol. Chem. 270:23700-23705, 1995, which is incorporated herein by reference). A homolog, the traE protein of plasmid RP4, has been described by Li, et al., J. Biol. Chem. 272:19582-19587 (1997) and can also be used in the practice of the invention. A DNA-protein adduct is formed with the enzyme covalently binding to the 5′-thymidine residue, with cleavage occurring between the two thymidine residues.

Type IB topoisomerases include the nuclear type I topoisomerases present in all eukaryotic cells and those encoded by vaccinia and other cellular poxviruses (see Cheng, et al., Cell 92:841-850, 1998, which is incorporated herein by reference). The eukaryotic type IB topoisomerases are exemplified by those expressed in yeast, Drosophila and mammalian cells, including human cells (see Caron and Wang, Adv. Pharmacol. 29B:271-297, 1994; Gupta, et al., Biochim. Biophys. Acta 1262:1-14, 1995, each of which is incorporated herein by reference; see, also, Berger, supra, 1998). Viral type EB topoisomerases are exemplified by those produced by the vertebrate poxviruses (vaccinia, Shope fibroma virus, ORF virus, fowlpox virus, and molluscum contagiosum virus), and the insect poxvirus (Amsacta moorei entomopoxvirus) (see Shuman, Biochim. Biophys. Acta 1400:321-337, 1998; Petersen, et al., Virology 230:197-206, 1997; Shuman and Prescott, Proc. Natl. Acad. Sci., USA 84:7478-7482, 1987; Shuman, J. Biol. Chem. 269:32678-32684, 1994; U.S. Pat. No. 5,766,891; PCT/US95/16099; PCT/US98/12372, each of which is incorporated herein by reference; see, also, Cheng, et al., supra, 1998).

Type II topoisomerases include, for example, bacterial gyrase, bacterial DNA topoisomerase IV, eukaryotic DNA topoisomerase II, and T-even phage encoded DNA topoisomerases (Roca and Wang, Cell 71:833-840, 1992; Wang, J. Biol. Chem. 266:6659-6662, 1991, each of which is incorporated herein by reference; Berger, supra, 1998;). Like the type IB topoisomerases, the type II topoisomerases have both cleaving and ligating activities. In addition, like type IB topoisomerase, substrate nucleic acid molecules can be prepared such that the type II topoisomerase can form a covalent linkage to one strand at a cleavage site. For example, calf thymus type II topoisomerase can cleave a substrate nucleic acid molecule containing a 5′ recessed topoisomerase recognition site positioned three nucleotides from the 5′ end, resulting in dissociation of the three nucleotide sequence 5′ to the cleavage site and covalent binding the of the topoisomerase to the 5′ terminus of the nucleic acid molecule (Andersen, et al., supra, 1991). Furthermore, upon contacting such a type II topoisomerase charged nucleic acid molecule with a second nucleotide sequence containing a 3′ hydroxyl group, the type II topoisomerase can ligate the sequences together, and then is released from the recombinant nucleic acid molecule. As such, type II topoisomerases also are useful for performing methods of the invention.

The various topoisomerases exhibit a range of sequence specificity. For example, type II topoisomerases can bind to a variety of sequences, but cleave at a highly specific recognition site (see Andersen, et al., J. Biol. Chem. 266:9203-9210, 1991, which is incorporated herein by reference.). In comparison, the type IB topoisomerases include site specific topoisomerases, which bind to and cleave a specific nucleotide sequence (“topoisomerase recognition site”). Upon cleavage of a nucleic acid molecule by a topoisomerase, for example, a type IB topoisomerase, the energy of the phosphodiester bond is conserved via the formation of a phosphotyrosyl linkage between a specific tyrosine residue in the topoisomerase and the 3′ nucleotide of the topoisomerase recognition site. Where the topoisomerase cleavage site is near the 3′ terminus of the nucleic acid molecule, the downstream sequence (3′ to the cleavage site) can dissociate, leaving a nucleic acid molecule having the topoisomerase covalently bound to the newly generated 3′ end.

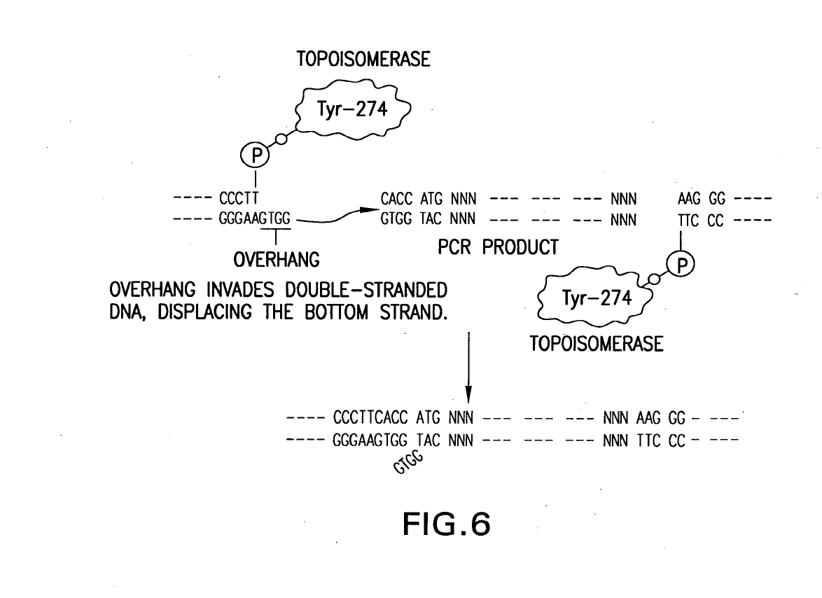

In particular embodiments, the 5′ termini of the ends of the nucleotide sequences to be linked by a type IB topoisomerase according to a method of certain aspects of the invention contain complementary 5′ overhanging sequences, which can facilitate the initial association of the nucleotide sequences, including, if desired, in a predetermined directional orientation. Alternatively, the 5′ termini of the ends of the nucleotide sequences to be linked by a type IB topoisomerase according to a method of certain aspects of the invention contain complementary 5′ sequences wherein one of the sequences contains a 5′ overhanging sequence and the other nucleotide sequence contains a complementary sequence at a blunt end of a 5′ terminus, to facilitate the initial association of the nucleotide sequences through strand invasion, including, if desired, in a predetermined directional orientation (FIG. 6). The term “5′ overhang” or “5′ overhanging sequence” is used herein to refer to a strand of a nucleic acid molecule that extends in a 5′ direction beyond the terminus of the complementary strand of the nucleic acid molecule. Conveniently, a 5′ overhang can be produced as a result of site specific cleavage of a nucleic acid molecule by a type LB topoisomerase.

In particular embodiments, the 3′ termini of the ends of the nucleotide sequences to be linked by a type IA topoisomerase according to a method of certain aspects of the invention contain complementary 3′ overhanging sequences, which can facilitate the initial association of the nucleotide sequences, including, if desired, in a predetermined directional orientation. Alternatively, the 3′ termini of the ends of the nucleotide sequences to be linked by a topoisomerase (e.g., a type IA or a type II topoisomerase) according to a method of certain aspects of the invention contain complementary 3′ sequences wherein one of the sequences contains a 3′ overhanging sequence and the other nucleotide sequence contains a complementary sequence at a blunt end of a 3′ terminus, to facilitate the initial association of the nucleotide sequences through strand invasion, including, if desired, in a predetermined directional orientation. The term “3′ overhang” or “3′ overhanging sequence” is used herein to refer to a strand of a nucleic acid molecule that extends in a 3′ direction beyond the terminus of the complementary strand of the nucleic acid molecule. Conveniently, a 3′ overhang can be produced upon cleavage by a type IA or type II topoisomerase.

The 3′ or 5′ overhanging sequences can have any sequence, though generally the sequences are selected such that they allow ligation of a predetermined end of one nucleic acid molecule to a predetermined end of a second nucleotide sequence according to a method of the invention. As such, while the 3′ or 5′ overhangs can be palindromic, they generally are not because nucleic acid molecules having palindromic overhangs can associate with each other, thus reducing the yield of a ds recombinant nucleic acid molecule covalently linked in both strands comprising two or more nucleic acid molecules in a predetermined orientation.

Any number of methods may be used to add topoisomerase cleavage sites to nucleic acid molecules and/or generate nucleic acid molecules to which topoisomerase is covalently bound. Examples of such methods are set out below in Example 8 and in U.S. Patent Publication No. 2003-0186233, the entire disclosure of which is incorporated herein by reference.

Suppressor tRNAs

Mutant tRNA molecules that recognize what are ordinarily stop codons suppress the termination of translation of an mRNA molecule and are termed suppressor tRNAs. Three codons are used by both eukaryotes and prokaryotes to signal the end of gene. When transcribed into mRNA, the codons have the following sequences: UAG (amber), UGA (opal) and UAA (ochre). Under most circumstances, the cell does not contain any tRNA molecules that recognize these codons. Thus, when a ribosome translating an mRNA reaches one of these codons, the ribosome stalls and falls of the RNA, terminating translation of the mRNA. The release of the ribosome from the mRNA is mediated by specific factors (see S. Mottagui-Tabar, Nucleic Acids Research 26(11), 2789, 1998). A gene with an in-frame stop codon (TAA, TAG, or TGA) will ordinarily encode a protein with a native carboxy terminus. However, suppressor tRNAs can result in the insertion of amino acids and continuation of translation past stop codons.

A number of such suppressor tRNAs have been found. Examples include, but are not limited to, the supE, supP, supD, supF and supZ suppressors, which suppress the termination of translation of the amber stop codon, supB, glT, supL, supN, supC and supM suppressors, which suppress the function of the ochre stop codon and glyT, trpT and Su-9 suppressors, which suppress the function of the opal stop codon. In general, suppressor tRNAs contain one or more mutations in the anti-codon loop of the tRNA that allows the tRNA to base pair with a codon that ordinarily functions as a stop codon. The mutant tRNA is charged with its cognate amino acid residue and the cognate amino acid residue is inserted into the translating polypeptide when the stop codon is encountered. For a more detailed discussion of suppressor tRNAs, the reader may consult Eggertsson, et al., (1988) Microbiological Review 52(3):354-374, and Engleerg-Kukla, et al. (1996) in Escherichia coli and Salmonella Cellular and Molecular Biology, Chapter 60, pps 909-921, Neidhardt, et al. eds., ASM Press, Washington, D.C.

Mutations that enhance the efficiency of termination suppressors, i.e., increase the read through of the stop codon, have been identified. These include, but are not limited to, mutations in the uar gene (also known as the prfA gene), mutations in the ups gene, mutations in the sueA, sueB and sueC genes, mutations in the rpsD (ramA) and rpsE (spcA) genes and mutations in the rplL gene.

Under ordinary circumstances, host cells would not be expected to be healthy if suppression of stop codons is too efficient. This is because of the thousands or tens of thousands of genes in a genome, a significant fraction will naturally have one of the three stop codons; complete read-through of these would result in a large number of aberrant proteins containing additional amino acids at their carboxy termini. If some level of suppressing tRNA is present, there is a race between the incorporation of the amino acid and the release of the ribosome. Higher levels of tRNA may lead to more read-through although other factors, such as the codon context, can influence the efficiency of suppression.

Organisms ordinarily have multiple genes for tRNAs. Combined with the redundancy of the genetic code (multiple codons for many of the amino acids), mutation of one tRNA gene to a suppressor tRNA status does not lead to high levels of suppression. The TAA stop codon is the strongest, and most difficult to suppress. The TGA is the weakest, and naturally (in E. coli) leaks to the extent of 3%. The TAG (amber) codon is relatively tight, with a read-through of ˜1% without suppression. In addition, the amber codon can be suppressed with efficiencies on the order of 50% with naturally occurring suppressor mutants. Suppression in some organisms (e.g., E. coli) may be enhanced when the nucleotide following the stop codon is an adenosine. Thus, the present invention contemplates nucleic acid molecules having a stop codon followed by an adenosine (e.g., having the sequence TAGA, TAAA, and/or TGAA).

Suppression has been studied for decades in bacteria and bacteriophages. In addition, suppression is known in yeast, flies, plants and other eukaryotic cells including mammalian cells. For example, Capone, et al. (Molecular and Cellular Biology 6(9):3059-3067, 1986) demonstrated that suppressor tRNAs derived from mammalian tRNAs could be used to suppress a stop codon in mammalian cells. A copy of the E. coli chloramphenicol acetyltransferase (cat) gene having a stop codon in place of the codon for serine 27 was transfected into mammalian cells along with a gene encoding a human senne tRNA that had been mutated to form an amber, ochre, or opal suppressor derivative of the gene. Successful expression of the cat gene was observed. An inducible mammalian amber suppressor has been used to suppress a mutation in the replicase gene of polio virus and cell lines expressing the suppressor were successfully used to propagate the mutated virus (Sedivy, et al., Cell 50: 379-389 (1987)). The context effects on the efficiency of suppression of stop codons by suppressor tRNAs has been shown to be different in mammalian cells as compared to E. coli (Phillips-Jones, et al, Molecular and Cellular Biology 15(12): 6593-6600 (1995), Martin, et al., Biochemical Society Transactions 21: (1993)) Since some human diseases are caused by nonsense mutations in essential genes, the potential of suppression for gene therapy has long been recognized (see Temple, et al., Nature 296(5857):537-40 (1982)). The suppression of single and double nonsense mutations introduced into the diphtheria toxin A-gene has been used as the basis of a binary system for toxin gene therapy (Robinson, et al., Human Gene Therapy 6:137-143 (1995)).

Use of Suppressor tRNAs to Conditionally Express Fusion Proteins

Because the methods used to create the nucleic acids of the invention are site specific, the orientation and/or reading frame of a nucleic acid sequence on a first nucleic acid molecule can be controlled with respect to the orientation and/or reading frame of a sequence on a second nucleic acid molecule when all or a portion of the molecules are joined in a recombination and/or topoisomerase-mediated reaction. This control makes the construction of fusions between sequences present on different nucleic acid molecules a simple matter.

In general terms, an open reading frame may be expressed in four forms: native at both amino and carboxy termini, modified at either end, or modified at both ends. The portion of a nucleic acid sequence encoding a polypeptide of interest may be referred to as an open reading frame (ORF). A nucleic acid sequence of interest comprising an ORF of interest may include the N-terminal methionine ATG codon, and a stop codon at the carboxy end, of the ORF, thus ATG-ORF-stop. Frequently, the nucleic acid molecule comprising the sequence of interest will include translation initiation sequences, tis, that may be located upstream of the ATG that allow expression of the gene, thus tis-ATG-ORF-stop. Constructs of this sort allow expression of an ORF as a protein that contains the same amino and carboxy amino acids as in the native, uncloned, protein. When such a construct is fused in-frame with an amino-terminal protein tag, e.g., GST, the tag will have its own tis, thus tis-ATG-tag-tis-ATG-ORF-stop, and the bases comprising the tis of the ORF will be translated into amino acids between the tag and the ORF. In addition, some level of translation initiation may be expected in the interior of the mRNA (i.e., at the ORF's ATG and not the tag's ATG) resulting in a certain amount of native protein expression contaminating the desired protein.

DNA (lower case): tis1-atg-tag-tis2-atg-orf-stop

RNA (lower case, italics): tis1-atg-tag-tis2-atg-orf-stop

Protein (upper case): ATG-TAG-TIS2-ATG-ORF (tis1 and stop are not translated)+contaminating ATG-ORF (translation of ORF beginning at tis2).

Using the methods disclosed herein, one skilled in the art can construct a vector containing a nucleic acid sequence encoding a polypeptide having a detectable activity (e.g., β-lactamase activity) adjacent to a recombination site permitting the in frame fusion of a nucleic acid sequence encoding a polypeptide having a detectable activity (e.g., β-lactamase activity) to the C- and/or N-terminus of the ORF of interest.

Given the ability to rapidly create a number of clones in a variety of vectors, there is a need in the art to maximize the number of ways a single cloned ORF can be expressed without the need to manipulate the construct itself. The present invention meets this need by providing materials and methods for the controlled expression of a C- and/or N-terminal fusion to a target ORF using one or more suppressor tRNAs to suppress the termination of translation at a stop codon. Thus, the present invention provides materials and methods in which a gene construct is prepared flanked with recombination sites.

The construct may be prepared with a sequence coding for a stop codon at the C-terminus of the ORF encoding the protein of interest. In some embodiments, a stop codon can be located adjacent to the ORF, for example, within the recombination site flanking the gene or at or near the 3′ end of the sequence of interest before a recombination site. The target gene construct can be transferred through recombination to various vectors that can provide various C-terminal or N-terminal tags (e.g., GFP, GST, His Tag, GUS, etc.) to the ORF of interest. In a particular embodiment of the invention, an ORF encoding a polypeptide of interest may be inserted into a vector comprising a nucleic acid sequence encoding a polypeptide having β-lactamase activity. When the stop codon is located at the carboxy terminus of the ORF, expression of the ORF with a “native” carboxy end amino acid sequence occurs under non-suppressing conditions (i.e., when the suppressor tRNA is not expressed) while expression of the ORF as a carboxy fusion protein occurs under suppressing conditions. Those skilled in the art will recognize that any suppressors and any codons could be used in the practice of the present invention. Suppressors may insert any amino acid at the position corresponding to the stop codon, for example, Ala, Arg, Asn, Asp, Cys, Gln, Glu, Gly, His, Ile, Leu, Lys, Met, Phe, Pro, Ser, Thr, Trp, Tyr, or Val may be inserted. In some embodiments, serine may be inserted.

In some embodiments, the gene coding for the suppressing tRNA may be incorporated into the vector from which the target ORF is to be expressed. In other embodiments, the gene for the suppressor tRNA may be in the genome of the host cell. In still other embodiments, the gene for the suppressor may be located on a separate nucleic acid molecule (e.g., plasmid, virus, linear nucleic acid molecule, etc.) and provided in trans. In embodiments of this type, the vector containing the suppressor gene may be a recombinant adenoviral vector and cells may be transduced with the viral vector.

In some embodiments, the nucleic acid molecule of the invention may be introduced into host cells as a vector (e.g., a plasmid, virus, etc) or may be stably integrated into the genome of the host cells. Suppressor tRNAs may be introduced into these cells using any of the methods described above.

More than one copy of a suppressor tRNA may be provided in all of the embodiments described herein. For example, a host cell may be provided that contains multiple copies of a gene encoding the suppressor tRNA. Alternatively, multiple gene copies of the suppressor tRNA under the same or different promoters may be provided in the same vector background as the target ORF of interest. In some embodiments, multiple copies of a suppressor tRNA may be provided in a different vector than the one containing the target ORF of interest. In other embodiments, one or more copies of the suppressor tRNA gene may be provided on the vector containing the ORF for the protein of interest and/or on another vector and/or in the genome of the host cell or in combinations of the above. When more than one copy of a suppressor tRNA gene is provided, the genes may be expressed from the same or different promoters that may be the same or different as the promoter used to express the ORF encoding the protein of interest.

In some embodiments, two or more different suppressor tRNA genes may be provided. In embodiments of this type one or more of the individual suppressors may be provided in multiple copies and the number of copies of a particular suppressor tRNA gene may be the same or different as the number of copies of another suppressor tRNA gene. Each suppressor tRNA gene, independently of any other suppressor tRNA gene, may be provided on the vector used to express the ORF of interest and/or on a different vector and/or in the genome of the host cell. A given tRNA gene may be provided in more than one place in some embodiments. For example, a copy of the suppressor tRNA may be provided on the vector containing the ORF of interest while one or more additional copies may be provided on an additional vector and/or in the genome of the host cell. When more than one copy of a suppressor tRNA gene is provided, the genes may be expressed from the same or different promoters that may be the same or different as the promoter used to express the ORF encoding the protein of interest and may be the same or different as a promoter used to express a different tRNA gene.

In some embodiments of the present invention, the target ORF of interest and the gene expressing the suppressor tRNA may be controlled by the same promoter. In other embodiments, the target ORF of interest may be expressed from a different promoter than the suppressor tRNA. Those skilled in the art will appreciate that, under certain circumstances, it may be desirable to control the expression of the suppressor tRNA and/or the target ORF of interest using a regulatable promoter. For example, either the target ORF of interest and/or the gene expressing the suppressor tRNA may be controlled by a promoter such as the lac promoter or derivatives thereof such as the tac promoter. In some embodiments, both the target ORF of interest and the suppressor tRNA gene are expressed from the T7 RNA polymerase promoter and, optionally, are expressed as part of one RNA molecule. In embodiments of this type, the portion of the RNA corresponding to the suppressor tRNA is processed from the originally transcribed RNA molecule by cellular factors.

In some embodiments, the expression of the suppressor tRNA gene may be under the control of a different promoter from that of the ORF of interest. In some embodiments, it may be possible to express the suppressor gene before the expression of the target ORF. This would allow levels of suppressor to build up to a high level, before they are needed to allow expression of a fusion protein by suppression of a the stop codon. For example, in embodiments of the invention where the suppressor gene is controlled by a promoter inducible with IPTG, the target ORF is controlled by the T7 RNA polymerase promoter and the expression of the T7 RNA polymerase is controlled by a promoter inducible with an inducing signal other than IPTG, e.g., NaCl, one could turn on expression of the suppressor tRNA gene with EPTG prior to the induction of the T7 RNA polymerase gene and subsequent expression of the ORF of interest. In some embodiments, the expression of the suppressor tRNA might be induced about 15 minutes to about one hour before the induction of the T7 RNA polymerase gene. In one embodiment, the expression of the suppressor tRNA may be induced from about 15 minutes to about 30 minutes before induction of the T7 RNA polymerase gene. In some embodiments, the expression of the T7 RNA polymerase gene is under the control of an inducible promoter.

In additional embodiments, the expression of the target ORF of interest and the suppressor tRNA can be arranged in the form of a feedback loop. For example, the target ORF of interest may be placed under the control of the T7 RNA polymerase promoter while the suppressor gene is under the control of both the T7 promoter and the lac promoter. The T7 RNA polymerase gene itself is also under the control of both the T7 promoter and the lac promoter. In addition, the T7 RNA polymerase gene has an amber stop mutation replacing a normal tyrosine codon, e.g., the 28th codon (out of 883). No active T7 RNA polymerase can be made before levels of suppressor are high enough to give significant suppression. Then expression of the polymerase rapidly rises, because the T7 polymerase expresses the suppressor gene as well as itself. In other embodiments, only the suppressor gene is expressed from the T7 RNA polymerase promoter. Embodiments of this type would give a high level of suppressor without producing an excess amount of T7 RNA polymerase. In other embodiments, the T7 RNA polymerase gene has more than one amber stop mutation. This will require higher levels of suppressor before active T7 RNA polymerase is produced.

In some embodiments of the present invention it may be desirable to have more than one stop codon suppressible by more than one suppressor tRNA. A recombinant nucleic acid molecule may be constructed so as to permit the regulatable expression of N- and/or C-terminal fusions of a protein of interest from the same construct. A nucleic acid molecule may comprise a first tag sequence expressed from a promoter and may include a first stop codon in the same reading frame as the tag. The stop codon may be located anywhere in the tag sequence and in particular may be located at or near the C-terminal of the tag sequence. The stop codon may also be located in a recombination site or in an internal ribosome entry sequence (IRES). The nucleic acid molecule may also include a sequence of interest which may comprising a ORF of interest that includes a second stop codon. The first tag and the ORF of interest may be in the same reading frame although inclusion of a sequence that causes frame shifting to bring the first tag into the same reading frame as the ORF of interest is within the scope of the present invention. The second stop codon may be in the same reading frame as the ORF of interest and may be located at or near the end of the coding sequence for the ORF. The second stop codon may optionally be located within a recombination site located 3′ to the sequence of interest. The construct may also include a second tag sequence in the same reading frame as the ORF of interest and the second tag sequence may optionally include a third stop codon in the same reading frame as the second tag. A transcription terminator and/or a polyadenylation sequence may be included in the construct after the coding sequence of the second tag. The first, second and third stop codons may be the same or different. In some embodiments, all three stop codons are different. In embodiments where the first and the second stop codons are different, the same construct may be used to express an N-terminal fusion, a C-terminal fusion and the native protein, by varying the expression of the appropriate suppressor tRNA. For example, to express the native protein, no suppressor tRNAs are expressed and protein translation is controlled by an appropriately located IRES. When an N-terminal fusion is desired, a suppressor tRNA that suppresses the first stop codon is expressed while a suppressor tRNA that suppresses the second stop codon is expressed in order to produce a C-terminal fusion. In some instances it may be desirable to express a doubly tagged protein of interest in which case suppressor tRNAs that suppress both the first and the second stop codons may be expressed.

### Host Cells

The invention also relates to host cells comprising one or more of the nucleic acid molecules invention containing one or more nucleic acid sequences encoding a polypeptide having a detectable activity and/or one or more other sequences of interest (e.g., two, three, four, five, seven, ten, twelve, fifteen, twenty, thirty, fifty, etc.). Representative host cells that may be used according to this aspect of the invention include, but are not limited to, bacterial cells, yeast cells, plant cells and animal cells. In particular embodiments, bacterial host cells include Escherichia spp. cells (particularly E. coli cells and most particularly E. coli strains DH10B, Stb12, DH5a, DB3, DB3.1 (e.g., E. coli LIBRARY EFFICIENCY® DB3.1™ Competent Cells; Invitrogen Corporation, Carlsbad, Calif.), DB4, DB5, JDP682 and ccdA-over (see U.S. application Ser. No. 09/518,188, filed Mar. 2, 2000, and U.S. provisional Application No. 60/475,004, filed Jun. 3, 2003, by Louis Leong et al., entitled “Cells Resistant to Toxic Genes and Uses Thereof,” the disclosures of which are incorporated by reference herein in their entireties); Bacillus spp. cells (particularly B. subtilis and B. megaterium cells), Streptomyces spp. cells, Erwinia spp. cells, Klebsiella spp. cells, Serratia spp. cells (particularly S. marcessans cells), Pseudomonas spp. cells (particularly P. aeruginosa cells), and Salmonella spp. cells (particularly S. typhimurium and S. typhi cells). Suitable animal host cells include insect cells (most particularly Drosophila melanogaster cells, Spodoptera frugiperda Sj9 and SJ21 cells and Trichoplusa High-Five cells), nematode cells (particularly C. elegans cells), avian cells, amphibian cells (particularly Xenopus laevis cells), reptilian cells, and mammalian cells (most particularly NIH3T3, 293, CHO, COS, VERO, BHK and human. cells). Suitable yeast host cells include Saccharomyces cerevisiae cells and Pichia pastoris cells. These and other suitable host cells are available commercially, for example, from Invitrogen Corporation, (Carlsbad, Calif.), American Type Culture Collection (Manassas, Va.), and Agricultural Research Culture Collection (NRRL; Peoria, Ill.).

Nucleic acid molecules to be used in the present invention may comprise one or more origins of replication (ORIs), and/or one or more selectable markers. In some embodiments, molecules may comprise two or more ORIs at least two of which are capable of functioning in different organisms (e.g., one in prokaryotes and one in eukaryotes). For example, a nucleic acid may have an ORI that functions in one or more prokaryotes (e.g., E. coli, Bacillus, etc.) and another that functions in one or more eukaryotes (e.g., yeast, insect, mammalian cells, etc.). Selectable markers may likewise be included in nucleic acid molecules of the invention to allow selection in different organisms. For example, a nucleic acid molecule may comprise multiple selectable markers, one or more of which functions in prokaryotes and one or more of which functions in eukaryotes.

Methods for introducing the nucleic acids molecules of the invention into the host cells described herein, to produce host cells comprising one or more of the nucleic acids molecules of the invention, will be familiar to those of ordinary skill in the art. For instance, the nucleic acid molecules of the invention may be introduced into host cells using well known techniques of infection, transduction, electroporation, transfection, and transformation. The nucleic acid molecules of the invention may be introduced alone or in conjunction with other nucleic acid molecules and/or vectors and/or proteins, peptides or RNAs. Alternatively, the nucleic acid molecules of the invention may be introduced into host cells as a precipitate, such as a calcium phosphate precipitate, or in a complex with a lipid. Electroporation also may be used to introduce the nucleic acid molecules of the invention into a host. Likewise, such molecules may be introduced into chemically competent cells such as E. coli. If the vector is a virus, it may be packaged in vitro or introduced into a packaging cell and the packaged virus may be transduced into cells. Thus nucleic acid molecules of the invention may contain and/or encode one or more packaging signal (e.g., viral packaging signals that direct the packaging of viral nucleic acid molecules). Hence, a wide variety of techniques suitable for introducing the nucleic acid molecules and/or vectors of the invention into cells in accordance with this aspect of the invention are well known and routine to those of skill in the art. Such techniques are reviewed at length, for example, in Sambrook, J., et al., Molecular Cloning, a Laboratory Manual, 2nd Ed., Cold Spring Harbor, N.Y.: Cold Spring Harbor Laboratory Press, pp. 16.30-16.55 (1989), Watson, J. D., et al., Recombinant DNA, 2nd Ed., New York: W.H. Freeman and Co., pp. 213-234 (1992), and Winnacker, E.-L., From Genes to Clones, New York: VCH Publishers (1987), which are illustrative of the many laboratory manuals that detail these techniques and which are incorporated by reference herein in their entireties for their relevant disclosures.

### Kits

In another aspect, the invention provides kits that may be used in conjunction with methods the invention. Kits according to this aspect of the invention may comprise one or more containers, which may contain one or more components selected from the group consisting of one or more nucleic acid molecules (e.g., one or more nucleic acid molecules comprising one or more nucleic acid sequence encoding a polypeptide having a detectable activity) of the invention, one or more primers, the molecules and/or compounds of the invention, one or more polymerases, one or more reverse transcriptases, one or more recombination proteins (or other enzymes for carrying out the methods of the invention), one or more topoisomerases, one or more buffers, one or more detergents, one or more restriction endonucleases, one or more nucleotides, one or more terminating agents (e.g., ddNTPs), one or more transfection reagents, pyrophosphatase, and the like. Kits of the invention may also comprise written instructions for carrying out one or more methods of the invention.

The present invention also provides kits that contain components useful for conveniently practicing the methods of the invention. In one embodiment, a kit of the invention contains a first nucleic acid molecule, which comprises a nucleic acid sequence encoding a polypeptide having a detectable activity, and contains one or more topoisomerase recognition sites and/or one or more covalently attached topoisomerase enzymes. Nucleic acid molecules according to this aspect of the invention may further comprise one or more recombination sites. In some embodiments, the nucleic acid molecule comprises a topoisomerase-activated nucleotide sequence. The topoisomerase-charged nucleic acid molecule may comprise a 5′ overhanging sequence at either or both ends and, the overhanging sequences may be the same or different. Optionally, each of the 5′ termini comprises a 5′ hydroxyl group.

In one embodiment, a kit of the invention contains a first nucleic acid molecule, which comprises a nucleic acid sequence encoding a polypeptide having a detectable activity, and contains one or more recombination sites. Nucleic acid molecules according to his aspect of the invention may further comprise one or more topoisomerase sites and/or topoisomerase enzymes.

In addition, the kit can contain at least a nucleotide sequence (or complement thereof) comprising a regulatory element, which can be an upstream or downstream regulatory element, or other element, and which contains a topoisomerase recognition site at one or both ends. In particular embodiments, kits of the invention contain a plurality of nucleic acid molecules, each comprising a different regulatory element or other element, for example, a sequence encoding a tag or other detectable molecule or a cell compartmentalization domain. The different elements can be different types of a particular regulatory element, for example, constitutive promoters, inducible promoters and tissue specific promoters, or can be different types of elements including, for example, transcriptional and translational regulatory elements, epitope tags, and the like. Such nucleic acid molecules can be topoisomerase-activated, and can contain 5′ overhangs or 3′ overhangs that facilitate operatively covalently linking the elements in a predetermined orientation, particularly such that a polypeptide such as a selectable marker is expressible in vitro or in one or more cell types.

The kit also can contain primers, including first and second primers, such that a primer pair comprising a first and second primer can be selected and used to amplify a desired ds recombinant nucleic acid molecule covalently linked in one or both strands, generated using components of the kit. For example, the primers can include first primers that are complementary to elements that generally are positioned at the 5′ end of a generated ds recombinant nucleic acid molecule, for example, a portion of a nucleic acid molecule comprising a promoter element, and second primers that are complementary to elements that generally are positioned at the 3′ end of a generated ds recombinant nucleic acid molecule, for example, a portion of a nucleic acid molecule comprising a transcription termination site or encoding an epitope tag. Depending on the elements selected from the kit for generating a ds recombinant nucleic acid molecule covalently linked in both strands, the appropriate first and second primers can be selected and used to amplify a full length functional construct.

In another embodiment, a kit of the invention contains a plurality of different elements, each of which can comprise one or more recombination sites and/or can be topoisomerase-activated at one or both ends, and each of which can contain a 5′-overhanging sequence or a 3′-overhanging sequence or a combination thereof. The 5′ or 3′ overhanging sequences can be unique to a particular element, or can be common to plurality of related elements, for example, to a plurality of different promoter element. In particular embodiments, the 5′ overhanging sequences of elements are designed such that one or more elements can be operatively covalently linked to provide a useful function, for example, an element comprising a Kozak sequence and an element comprising a translation start site can have complementary 5′ overhangs such that the elements can be operatively covalently linked according to a method of the invention.

The plurality of elements in the kit can comprise any elements, including transcription or translation regulatory elements; elements required for replication of a nucleotide sequence in a bacterial, insect, yeast, or mammalian host cell; elements comprising recognition sequences for site specific nucleic acid binding proteins such as restriction endonucleases or recombinases; elements encoding expressible products such as epitope tags or drug resistance genes; and the like. As such, a kit of the invention provides a convenient source of different elements that can be selected depending, for example, on the particular cells that a construct generated according to a method of the invention is to be introduced into or expressed in. The kit also can contain PCR primers, including first and second primers, which can be combined as described above to amplify a ds recombinant nucleic acid molecule covalently linked in one or both strands, generated using the elements of the kit. Optionally, the kit further contains a site specific topoisomerase in an amount useful for covalently linking in at least one strand, a first nucleic acid molecule comprising a topoisomerase recognition site to a second (or other) nucleic acid molecule, which can optionally be topoisomerase-activated nucleic acid molecules or nucleotide sequences that comprise a topoisomerase recognition site.

In still another embodiment, a kit of the invention contains a first nucleic acid molecule, which comprises a nucleic acid sequence encoding a polypeptide having a detectable activity, and contains a topoisomerase recognition site and/or a recombination site at each end; a first and second PCR primer pair, which can produce a first and second amplification products that can be covalently linked in one or both strands, to the first nucleic acid molecule in a predetermined orientation according to a method of the invention.

Kits of the invention may further comprise (1) instructions for performing one or more methods described herein and/or (2) a description of one or more compositions described herein. These instructions and/or descriptions may be in printed form. For example, these instructions and/or descriptions may be in the form of an insert which is present in kits of the invention.

It will be understood by one of ordinary skill in the relevant arts that other suitable modifications and adaptations to the methods and applications described herein are readily apparent from the description of the invention contained herein in view of information known to the ordinarily skilled artisan, and may be made without departing from the scope of the invention or any embodiment thereof. Having now described the present invention in detail, the same will be more clearly understood by reference to the following examples, which are included herewith for purposes of illustration only and are not intended to be limiting of the invention.

### EXAMPLES

### Example 1

The present invention provides a highly efficient cloning strategy for the direct insertion of amplified promoter sequences (for example, using Taq polymerase) into a reporter vector. One non-limiting example of materials suitable for the practice of the invention may be obtained from Invitrogen Corporation, Carlsbad, Calif. under the trade name pGeneBLAzer™ TOPO® TA Expression Kits. In one aspect, promoter sequences can be inserted into a nucleic acid molecule upstream of the β-lactamase reporter gene. Resultant nucleic acid molecules may then be transfected into suitable host cells (e.g., mammalian cells) and assayed for promoter function and strength in vivo or in vitro. β-lactamase activity may be determined using any technique known to those skilled in the art, for example, using the GeneBLAzer™ In Vivo or In Vitro Detection Kit, available from Invitrogen Corporation, Carlsbad, Calif. In contrast to previously employed methods, no ligase, post-PCR procedures, or PCR primers containing specific sequences are required.

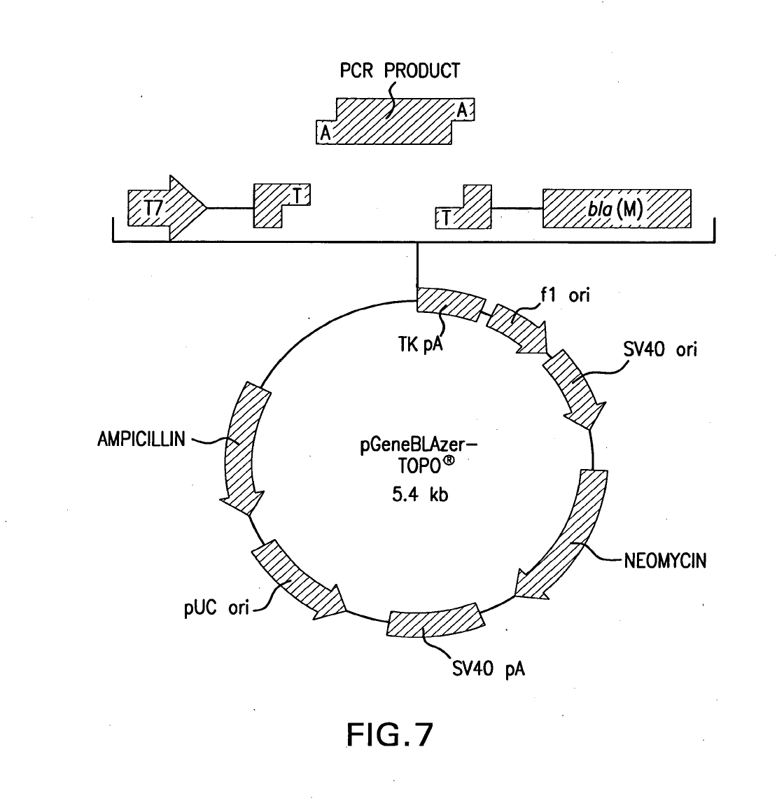

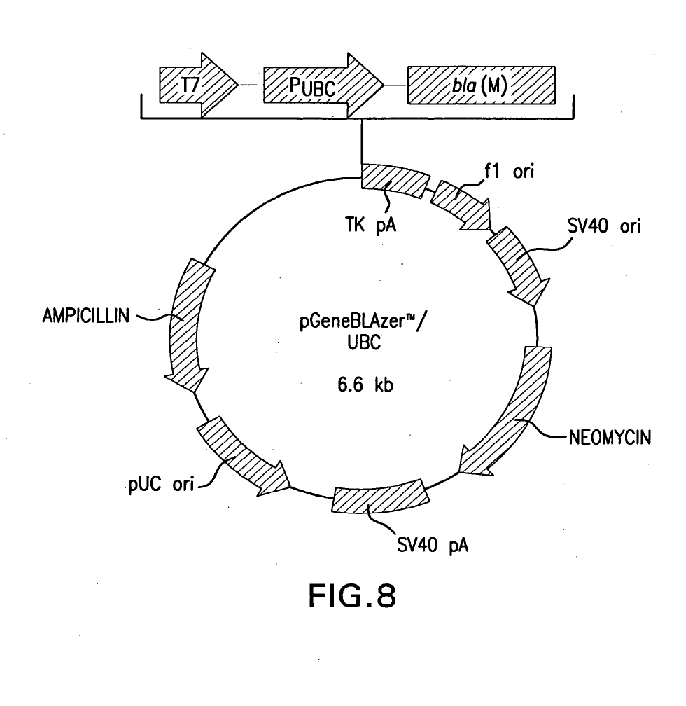

In some embodiments, suitable nucleic acid molecules for practicing the methods of the invention may be vectors (e.g., plasmid vectors such as pGeneBLAzer-TOPO™). FIG. 7 provides a vector map pGeneBLAzer-TOPO™ and Table 31 provides the nucleotide sequence of the vector. A vector suitable for practice of the present invention may include one or more of the following characteristics: one or more recognition sequences, for example, topisomerase recognition sequences that may be used to clone amplified nucleic acid molecules amplified using Taq polymerase; one or more nucleic acid sequences encoding a polypeptide having a detectable activity (e.g., encoding a β-lactamase such as bla(M)); one or more polyadenylation sequence for efficient transcription termination and polyadenylation of mRNA (e.g., Herpes Simplex Virus thymidine kinase (TK) polyadenylation sequence (see, Cole, C. N., and Stacy, T. P. (1985) Mol. Cell. Biol. 5, 2104-2113) or the SV40 polyadenylation sequence); one or more selectable markers, for example, the neomycin resistance gene for selection of stable cell lines (see, Southern, P. J., and Berg, P. (1982) J. Molec. Appl. Gen. 1, 327-339) and/or the ampicillin resistance gene for selection in E. coli; one or more origin of replication, for example, the pUC origin, which permits high-copy replication and maintenance of plasmids in E. coli and/or the f1 origin, which allows single-stranded DNA rescue, and/or the SV40 origin, which allows episomal replication in cells expressing the SV40 large T antigen; and/or one or more promoter sequences (e.g., SV40 early promoter, T7 promoter, etc.). The nucleic acid sequence of pGeneBLAzer-TOPO™ is provided in FIGS. 7B and 7C. Any nucleic acid sequence to be assayed for promoter activity may be used in conjunction with present invention. An example of a vector of the invention containing the ubiquitin promoter is pGeneBLAzer™/UbC. A map of this plasmid is provided as FIG. 8.

Materials and methods of the invention (e.g., GeneBLAzer™ Technology and the GeneBLAzer™ Detection System) may be used as described herein as a reporter of gene expression in mammalian cells. Materials and methods of the invention are suitable for use as a sensitive reporter of gene expression in living host cells (e.g., mammalian cells) using fluorescence microscopy. Materials and methods of the invention may provide a ratiometric readout that minimizes differences due to variability in cell number, substrate concentration, fluorescence intensity, and emission sensitivity. Materials and methods of the invention are compatible with a wide variety of in vivo and in vitro applications including microplate-based transcriptional assays and flow cytometry. Materials and methods of the invention provides flexible and simple assay development platforms for gene expression in host cells (e.g., mammalian cells). In particular, materials and methods of the invention may use a non-toxic substrate that allows continued cell culturing after quantitative analysis.

In a particular embodiment, methods of the invention may use one or more topoisomerase enzymes to join a nucleic acid sequence to be assayed for promoter activity to a nucleic acid sequence encoding a polypeptide having a detectable activity. A suitable example of a topoisomerase is topoisomerase I from Vaccinia virus, which binds to duplex DNA at specific sites and cleaves the phosphodiester backbone after 5′-CCCTT in one strand (see, Shuman, S. (1994) J. Biol. Chem. 269, 32678-32684). The energy from the broken phosphodiester backbone is conserved by formation of a covalent bond between the 3′ phosphate of the cleaved strand and a tyrosyl residue (Tyr-274) of topoisomerase I. The phospho-tyrosyl bond between the DNA and enzyme can subsequently be attacked by the 5′ hydroxyl of the original cleaved strand, reversing the reaction and releasing topoisomerase. TOPO® Cloning exploits this reaction to efficiently clone PCR products.

In a particular embodiment, the pGeneBLAzer-TOPO® vector is linearized and has single 3′ thymidine (T) overhangs for TA Cloning®. In embodiments of this type, topoisomerase I may be covalently bound to the vector (this is referred to as “activated vector”). As is known in the art, Taq polymerase has a nontemplate-dependent terminal transferase activity that adds a single deoxyadenosine (A) to the 3′ ends of PCR products. In a particular embodiment, a linearized vector may be supplied (e.g., as a component in a kit) and may have overhanging 3′ deoxythymidine (T) residues. Embodiments of this type may allow PCR products to ligate efficiently into the vector. Ligation of the vector with a PCR product containing 3′ A-overhangs is very efficient and occurs spontaneously within 5 minutes at room temperature. After the topoisomerase-mediated joining of the nucleic acid molecules, the resultant nucleic acid molecule may be introduced into a suitable host cell (e.g., transformed into chemically competent cells or electroporated directly into electrocompetent cells).

The materials and methods of the invention facilitate fluorescent detection of β-lactamase reporter activity in host cells (e.g., mammalian cells). In some embodiments, materials of the invention may comprise a β-lactamase reporter gene, bla(M), a truncated form of the E. coli bla gene. When filsed to promoter sequences (e.g., in the pGeneBLAzer-TOPO® vector), the bla(M) gene functions as a reporter of promoter activity in host cells (e.g., mammalian cells).

Materials and methods of the of the invention may also comprise one or more fluorescence resonance energy transfer (FRET)-enabled substrates (e.g., CCF2) to facilitate fluorescence detection of β-lactamase reporter activity. In the absence or presence of β-lactamase reporter activity, cells loaded with the CCF2 substrate fluoresce green or blue, respectively. Comparing the ratio of blue to green fluorescence in a population of live cells or in a cell extract prepared from a sample to a negative control provides a means to quantitate gene expression.

In some embodiments, a β-lactamase for use in the present invention may be the product encoded by the ampicillin resistance gene (bla), which is the bacterial enzyme that hydrolyzes penicillins and cephalosporins. The bla gene is present in many cloning vectors and allows ampicillin selection in E. coli. β-lactamase is not found in mammalian cells.

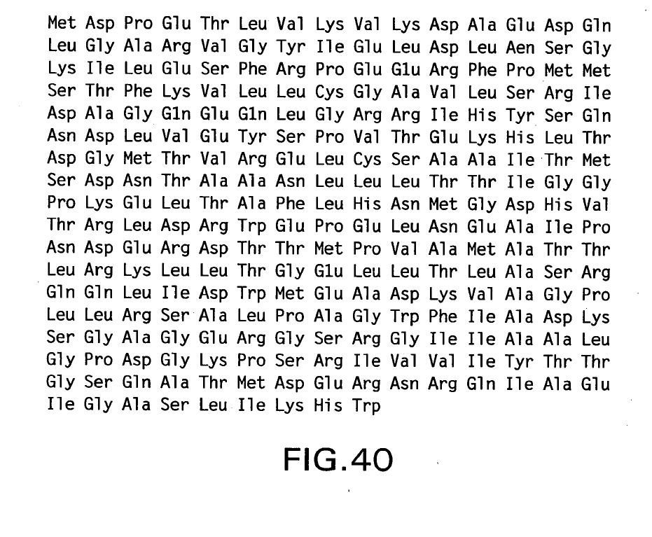

In some embodiments, materials and methods of the invention may use a modified bla gene as a reporter in mammalian cells. One example is a bla gene derived from the E. coli TEM-1 gene present in many cloning vectors (see, Zlokarnik, et al. (1998) Science 279, 84-88), which has been modified in that 72 nucleotides encoding the first 24 amino acids of β-lactamase were deleted from the N-terminal region of the gene. These 24 amino acids comprise the bacterial periplasmic signal sequence, and deleting this region allows cytoplasmic expression of β-lactamase in mammalian cells. The amino acid at position 24 was mutated from His to Asp to create an optimal Kozak sequence for improved translation initiation. As used herein, this modified reporter gene is named bla(M) and the maino acid sequence is provided in FIG. 40. The TEM-1 gene also contains 2 mutations (at nucleotide positions 452 and 753) that distinguish it from the bla gene in pBR322 (see, Sutcliffe, J. G. (1978) Proc. Nat. Acad. Sci. USA 75, 3737-3741).

Methods of the invention may comprise designing PCR primers to amplify a desired nucleic acid sequence to be assayed as for promoter activity; amplifying the desired nucleic acid sequence; cloning the nucleic acid sequence into a vector of the invention (e.g., pGeneBLAzer-TOPO®). Methods may further entail transforming the topoisomerase-mediated cloning reaction into competent cells (e.g., One Shot® TOP10 E. coli, Invitrogen Corporation, Carlsbad, Calif.) and selecting for transformants on LB agar plates containing 100 μg/ml ampicillin. Transformants can be screened for the presence and orientation of the nucleic acid sequence to be assayed for promoter activity using standard techniques, for example, by restriction digestion, PCR, or sequencing. A plasmid having the correct nucleic acid sequence in the correct orientation may be purified for transfection. The purified plasmid may be introduced into a suitable host cell. A stable cell line containing the plasmid may be isolated. Transformed host cells may be assayed for β-lactamase activity, for example, using the appropriate GeneBLAzer™ Detection Kit.

In particular embodiments, materials and methods of the invention may be used to analyze one or more of tissue and cell-specific promoter function, transcriptional enhancers in a known promoter, and/or deletions within a promoter. One skilled in the art will appreciate that when analyzing promoters in a reporter vector, it is important to realize that sequences within the native gene can influence regulation of its own promoter. In addition, sequences within the reporter gene can also affect transcription from the promoter under study. It is recommended that any observations of transcriptional control of the fusion gene be verified by comparison with expression of the native gene expressed from the same promoter. Techniques well known in the art (e.g., S1 mapping) can be used to confirm that the subcloned promoter initiates transcription at the correct site. For more information about S1 mapping, see Ausubel, et al. (1994) Current Protocols in Molecular Biology, pages 4.6.1 to 4.6.13, New York: Greene Publishing Associates and Wiley-Interscience. Since initiation of translation in eukaryotes occurs at the first available AUG codon, it is important that there are no AUG codons between the start of transcription and the AUG of the reporter gene.

The selection of suitable primers for use in the amplification of a sequence of interest to be assayed for promoter activity is routine in the art. Unique restriction sites may be included in the 5′ and 3′ primers to excise the fragment or facilitate analysis once it is TOPO® Cloned. Primers for the amplification of a sequence of interest should not be 5′-phosphorylated. Phosphates will inhibit topoisomerase I and the synthesized PCR product will not ligate into the pGeneBLAzer-TOPO® vector. FIG. 9 shows the insertion region in an exemplary vector of the invention. In some embodiments, vectors of the invention (e.g., pGeneBLAzer-TOPO®) may be supplied linearized between base pair 116 and 117 at the TOPO Cloning site.

Any suitable DNA polymerase or combination of DNA polymerases may be used to amplify the sequence of interest. For example, mixtures of Taq polymerase and a proofreading polymerase (e.g., Pfu DNA polymerase) may be used. When mixtures are used, Taq may be used in excess of a 10:1 ratio to ensure the presence of 3′ A-overhangs on the PCR product. One suitable DNA polymerase for use in methods of the invention is Platinum® Taq DNA Polymerase High Fidelity available from Invitrogen Corporation, Carlsbad, Calif. If mixtures are used that do not have enough Taq polymerase or a proofreading polymerase is used without Taq polymerase, 3′ A-overhangs can be added after amplification. One suitable method for adding 3′-A overhangs is to add Taq DNA polymerase to the amplification reaction mixture. For example, 0.7-1 unit of Taq polymerase may be added to each tube and then the tubes may be incubated under suitable conditions to allow addition of 3′-A by Taq polymerase. One non-limiting example of suitable conditions is to add Taq polymerase to the tube containing the amplification reaction and to incubate at 72° C. for 8-10 minutes without cycling the temperature. Typically, it is not necessary to purify the amplification product or change buffers prior to the addition of Taq polymerase.

One skilled in the art can select suitable amplification conditions for a sequence of interest using routine experimentation. One example of a suitable set of amplification conditions follows. A 50 μl PCR reaction may be set up, for example, containing 10-100 ng DNA Template, 5 μl of 10×PCR Buffer, 0.5 μl of 50 mM dNTPs, 100-200 ng of each primer, sterile water can be added to a final volume of 49 μl, and 1 μl of Taq Polymerase at a concentration of 1 unit/μl can be added.

As will be appreciated by those skilled in the art, these conditions may be varied. For example, less DNA may be used if plasmid DNA is used as a template and more DNA may be used if genomic DNA is used as a template. Selection of suitable cycling parameters (e.g., time and temperature of annealing and extension reactions) are routine in the art and may be adjusted for any specific primers and template.

A 7 to 30 minute extension at 72° C. after the last cycle may be used to ensure that all PCR products are full length and 3′ adenylated. The amplification product may be checked, for example, by agarose gel electrophoresis. Conditions may adjusted to produce a single, discrete band on an agarose gel. If samples are to be stored (e.g., overnight) before proceeding with TOPO® Cloning, samples may be extracted with phenol-chloroform to remove the polymerases. After phenol-chloroform extraction, the DNA may be precipitated with ethanol and resuspended in TE buffer to the starting volume of the amplification reaction.

Optionally, if the amplification reaction does not produce a single discrete band, the amplification product may be purified, for example, from an agarose gel prior to insertion into nucleic acid molecule of the invention. When an amplification product is to be purified, nuclease contamination and long exposure to UV light should be avoided.

Alternatively, the amplification conditions may be varied to eliminate multiple bands and smearing as is known in the art (see, for example, Innis, et al. (1990) PCR Protocols: A Guide to Methods and Applications, Academic Press, San Diego, Calif.) Commercially available materials may be used to optimize the amplification reaction, for example, The PCR Optimizer™ Kit (Catalog no. K1220-01) is available from Invitrogen Corporation, Carlsbad, Calif.

In some embodiments, salt may be included in a topoisomerase reaction to join a nucleic acid molecule having a sequence of interest and a nucleic acid molecule having a nucleic acid sequence encoding a polypeptide having a detectable activity. For example, including salt (200 mM NaCl, 10 mM MgCl₂) in the TOPO® Cloning reaction increases the number of transformants 2- to 3-fold. In the presence of salt, incubation times of greater than 5 minutes can also increase the number of transformants. This is in contrast to experiments without salt where the number of transformants decreases as the incubation time increases beyond 5 minutes. Without wishing to be bound by theory, including salt allows for longer incubation times because it prevents topoisomerase I from rebinding and potentially nicking the DNA after ligating the PCR product and dissociating from the DNA. The result is more intact molecules, leading to higher transformation efficiencies.

One skilled in the art will appreciate that the amount of salt that may be added to a topoisomerase reaction may vary depending on the method used to introduced the topoisomerase joined nucleic acid molecules into a host cell. For TOPO® Cloning and transformation into chemically competent E. coli, adding sodium chloride and magnesium chloride to a final concentration of 200 mM NaCl, 10 mM MgCl₂ in the TOPO® Cloning reaction may increase the number of colonies over time. A salt solution (e.g., 1.2 M NaCl; 0.06 M MgCl₂) can be used to adjust the TOPO® Cloning reaction to the recommended concentration of NaCl and MgCl₂. For transformation of electrocompetent E. coli, the amount of salt in the TOPO® Cloning reaction must be reduced to 50 mM NaCl, 2.5 mM MgCl₂ to prevent arcing. For example, the salt solution can be diluted 4-fold with sterile water to prepare a 300 mM NaCl, 15 mM MgCl₂ solution for convenient addition to the TOPO® Cloning reaction.

Suitable conditions for topoisomerase-mediated joining of nucleic acid molecules are known to those skilled in the art. Non-limiting examples of suitable conditions follow. When the joined nucleic acid molecules are to be introduced into a competent host cell by transformation, a suitable set of conditions is a 6 μl reaction volume containing 0.5 to 41l of amplification product, 1 μl of the a 1.2 M NaCl; 0.06 M MgCl₂ salt solution, sterile water to a final volume of 5 μl, and 1 μl of topoisomerase charged vector. For electroporation, 1 μl of a 1:4 dilution of the 1.2 M NaCl; 0.06 M MgCl₂ may be used. The reagents may be added and gently mixed and incubated for 5 minutes at room temperature. For most applications, 5 minutes will yield sufficient colonies for analysis. The length of the TOPO® Cloning reaction can be varied from 30 seconds to 30 minutes. For routine subcloning of PCR products, 30 seconds may be sufficient. For large PCR products (>1 kb) or if TOPO® Cloning a pool of PCR products, increasing the reaction time will yield more colonies. After incubation at room temperature, the reaction mixture may be placed on ice and the joined nucleic acid molecules may be introduced into a suitable host cell using standard techniques. Optionally, the TOPO® Cloning reaction can be stored at −20° C. overnight.

Other factors that are known to those skilled in the art may impact the efficiency with which a nucleic acid molecule having a nucleic acid sequence to be assayed for promoter activity is joined with a nucleic acid molecule having a nucleic acid sequence encoding a polypeptide having a detectable activity. For example, the pH of the amplification reaction may affect the amount of amplification product produced. If the pH of the PCR reaction is too high, the pH of the PCR amplification reaction may be adjusted with 1 M Tris-HCl, pH 8. Another factor is incomplete extension during PCR. This may be adjusted by including a final extension step of 7 to 30 minutes during PCR. Longer PCR products will need a longer extension time. Note that Taq polymerase is less efficient at adding a nontemplate 3′ A next to another A. Taq is most efficient at adding a nontemplate 3′ A next to a C. Primers may be designed so that they contain a 5′ G instead of a 5′ T (see, Brownstein, et al. (1996) BioTechniques 20, 1004-1010). When cloning large inserts (>3 kb), it may be desirable to gel-purify the insert. The amount of PCR product may be adjusted by concentrating or diluting the PCR product as needed. Up to 4 μl of the PCR reaction may be added to the TOPO® Cloning reaction. If false positives are observed, it may be desirable to gel purify the PCR product. The size of promoter sequences cloned can impact the efficiency. For large plasmids, electroporation to transform into E. coli may provide an increased number of colonies. Electrocompetent TOP10 cells are commercially available from Invitrogen Corporation, Carlsbad, Calif.

One skilled in the art will appreciate that the above-described protocol can be varied. For example, the amount of time spent on various steps can be varied. For example, the TOPO® Cloning reaction may be incubated for only 30 seconds instead of 5 minutes. When TOPO® Cloning large PCR products, toxic genes, or cloning a pool of PCR products, it may be desirable to produce more transformants to obtain the desired clones. To increase the number of colonies the salt-supplemented TOPO® Cloning reaction may be incubated for longer time (e.g., for 20 to 30 minutes instead of 5 minutes). Increasing the incubation time of the salt-supplemented TOPO® Cloning reaction allows more molecules to ligate, increasing the transformation efficiency. Addition of salt appears to prevent topoisomerase from rebinding and nicking the DNA after it has ligated the PCR product and dissociated from the DNA. To clone dilute PCR products, it may be desirable to increase the amount of the PCR product used, incubate the TOPO® Cloning reaction for 20 to 30 minutes, and/or concentrate the PCR product by precipitation.

Any protocol used to introduce nucleic acid molecules into host cells known to those skilled in the art may be used. Chemically competent cells may be made using standard techniques or commercially available cells may be used. An example of a suitable protocol for introducing nucleic acid molecules into commercially available competent cells is as follows. 2 μl of the TOPO® Cloning reaction from above may be added to a vial of One Shot® TOP10 Chemically Competent E. coli (Invitrogen Corporation, Carlsbad, Calif.) and mixed gently. The cells should not be mixed by pipetting up and down. The nucleic acid molecule: cell mixture may be incubated on ice for 5 to 30 minutes. Longer incubations on ice seem to have a minimal effect on transformation efficiency. The length of the incubation may be varied. The mixture may be heat shocked for 30 seconds at 42° C. without shaking. After heat shock, the mixture should be immediately transferred to ice. 250 μl of room temperature S.O.C. medium may be added. The tube may be tightly capped and shaken horizontally (200 rpm) at 37° C. for 1 hour. 25-200 μl from each transformation may be spread on a pre-warmed selective plate and incubated overnight at 37° C. Two different volumes (e.g., 20 μl and 200 μl) can be plated to ensure that at least one plate will have well-spaced colonies. An efficient TOPO® Cloning reaction will produce hundreds of colonies. Pick ˜10 colonies for analysis.

Any protocol used to introduce nucleic acid molecules into host cells known to those skilled in the art may be used. Electrocompetent cells may be made using standard techniques or commercially available cells may be used. An example of a suitable protocol for introducing nucleic acid molecules into electrocompetent cells is as follows. 2 μl of the TOPO® Cloning reaction described above may be added to 50 μl of electrocompetent E. coli and mixed gently. The cells should not be mixed by pipetting up and down. The formation of bubbles should be avoided. The mixture of DNA and electrocompetent cells can be transferred into a 0.1 cm cuvette. Electroporate samples using standard protocols and settings. 250 μl of room temperature S.O.C. medium may be added immediately. The solution can be transferred to a 15 ml snap-cap tube (i.e. Falcon) and shaken for at least 1 hour at 37° C. to allow expression of the antibiotic resistance marker. 10-50 μl from each transformation can be spread on a pre-warmed selective plates and incubated overnight at 37° C. To ensure even spreading of small volumes, add 20 μl of S.O.C. Medium. Two different volumes may be plated to ensure that at least one plate will have well-spaced colonies. An efficient TOPO® Cloning reaction will produce hundreds of colonies. Pick ˜10 colonies for analysis.

Individual colonies may be picked and overnight cultures grown (e.g., 3-5 mL cultures in LB medium containing 100 μg/mL ampicillin). Plasmids may be isolated using standard techniques. Analyze transformants for the presence of the sequence of interest to be assayed for promoter activity using any technique known in the art (e.g., restriction digests, sequencing, PCR, etc.). For example, the sequence of the pGeneBLAzer TOPO® vector is provided above. Primers can be designed from the sequence provided to sequence or PCR amplify a sequence of interest inserted into the vector to verify the presence of the sequence of interest in the selected clones.

Once a desired nucleic acid molecule has been produced in which a sequence of interest to be assayed for promoter activity is operably joined to a sequence encoding a polypeptide having a detectable activity, the desired nucleic acid molecule, which may be a plasmid, may be introduced into a suitable host cell (e.g., a mammalian cell). Plasmid DNA for transfection into eukaryotic cells must be very clean and free from phenol and sodium chloride. Contaminants will kill the cells and salt will interfere with lipid complexing decreasing transfection efficiency. Plasmid DNA (up to 200 μg) may be isolated using the S.N.A.P.™ MidiPrep Kit (Invitrogen Corporation, Carlsbad, Calif., Catalog no. K1910-01) or CsCl gradient centrifugation.

For analysis of promoter activity of a sequence of interest, positive and negative controls may be included to evaluate expression and detection β-lactamase. A negative control can be either a mock transfection or a pGeneBLAzer-TOPO® construct containing non-promoter DNA sequences (i.e. stuffer DNA).

For a positive control, the pGeneBLAzer m/UbC plasmid described above may be used. In this vector, the human ubiquitin C (UbC) promoter (see, Nenoi, et al. (1996) Gene 175, 179-185) controls expression of the β-lactamase reporter gene. This plasmid may be propagated by transformation into a recA, endA E. coli strain such as TOP10, DH5a, or equivalent. Transformants can be selected on LB agar plates containing 100 μg/ml ampicillin. A glycerol stock of a transformant containing plasmid may be prepared for long-term storage.

Nucleic acid molecules may be introduced into host cells using any technique known to those skilled in the art. Transfection protocols may be determined empirically or may be obtained from original references or the supplier of the cell line. Factors that may influence the transfection efficiency of a host cell include, but are not limited to, medium requirements, timing of passaging of cells, and the dilution of the cells when passaged. Further information is provided in Ausubel, (1994) Current Protocols in Molecular Biology. Suitable transfection methods for include calcium phosphate (see, Chen, C., and Okayama, H. (1987) Mol. Cell. Biol. 7, 2745-2752, Wigler, et al. (1977) Cell 11, 223-232), lipid-mediated (see, Felgner, et al. (1989) Proc. West. Pharmacol Soc. 32, 115-121; and Felgner, P. L. and Ringold, G. M. (1989) Nature 337, 387-388), and electroporation (see, Chu, et al. (1987) Nucleic Acids Res. 15, 1311-1326; and Shigekawa, K., and Dower, W. J. (1988) BioTechniques 6, 742-751). One suitable transfection reagent is Lipofectamine™ 2000 (Invitrogen Corporation, Carlsbad, Calif. Catalog no. 11668-027).

In some embodiments, nucleic acid molecules produced using methods of the invention may be used to generate stable cell lines comprising the nucleic acid molecules. For example, nucleic acid molecules of the invention may comprise a selectable marker that may be used to selct for cells containing the nucleic acid molecule of the invention. One example is the pGeneBLAzer-TOPO® vector that contains the neomycin resistance gene to allow selection of stable cell lines using Geneticin®. To create stable cell lines, transfect the nucleic acid molecule of the invention into the host cell line (e.g., mammalian cell line) of choice and select for foci using Geneticin®. Geneticin® blocks protein synthesis in mammalian cells by interfering with ribosomal function. It is an aminoglycoside, similar in structure to neomycin, gentamycin, and kanamycin. Expression in mammalian cells of the bacterial aminoglycoside phosphotransferase gene (APH), derived from Tn5, results in detoxification of Geneticin® (see, Southern, P. J., and Berg, P. (1982) J. Molec. Appl. Gen. 1, 327-339).

Geneticin® is commercially available (e.g., from Invitrogen Corporation, Carlsbad, Calif.). A stock of Geneticin® may be prepared (e.g., 50 mg/ml in buffer such as 100 mM HEPES, pH 7.3). Sufficient stock solution may be added to bring the concentration in the medium to about 100 to about 1000 μg/ml of Geneticin® in complete growth medium. Varying concentrations of Geneticin®0 may be tested on particular cell lines to determine the concentration that kills the particular cell line (i.e., a kill curve). Cells differ in their susceptibility to Geneticin®. Cells will divide once or twice in the presence of lethal doses of Geneticin®, so the effects of the drug take several days to become apparent. Complete selection can take from 2 to 4 weeks of growth in selective medium.

Once an appropriate Geneticin® concentration has been determined for a particular cell line, a stable cell line comprising an nucleic acid molecule of the invention may be prepared. For example, a cell line of interest may be transfected with a nucleic acid molecule of the invention using any transfection method known to those in the art. 24 hours after transfection, the cells may be washed and fresh growth medium added. 48 hours after transfection, the cells may be split into fresh growth medium such that they are no more than 25% confluent. If the cells are too dense, the antibiotic will not kill the cells. Antibiotics work best on actively dividing cells. The cells may be incubated at 37° C. for 2-3 hours until they have attached to the culture dish. The growth medium may be removed and replaced with fresh growth medium containing the Geneticin® at the pre-determined concentration required the particular cell line. The cells may be fed with selective media every 3-4 days until Geneticin®-resistant colonies can be identified. Multiple (e.g., five or more) Geneticin®-resistant colonies can be picked and expanded to assay.

Any suitable assay may be used to detect the activity of the polypeptide having a detectable activity. One suitable assay is described below for the case when the polypeptide has β-lactamase activity.

### Example 2

In some embodiments, polypeptides having a detectable activity according to the present invention may have a β-lactamase activity. β-lactamase activity may be detected using any technique known to those skilled in the art. Kits for the detection of β-lactamase activity are commercially available, for example, GeneBLAzer™ Detection Kits from Invitrogen Corporation, Carlsbad, Calif.

Materials and methods of the invention facilitate fluorescent detection of β-lactamase reporter activity in host cells (e.g., mammalian cells). In one aspect, materials of the invention may include one or more polypeptides having β-lactamase activity as described above. Such polypeptides can be used as reporters of promoter activity or gene expression in mammalian cells, respectively. Materials of the invention may also include one or more fluorescence resonance energy transfer (FRET)-enabled substrates (e.g., CCF2), which facilitate fluorescent detection of β-lactamase reporter activity. In the absence or presence of β-lactamase reporter activity, cells loaded with the CCF2 substrate fluoresce green or blue, respectively. Comparing the ratio of blue to green fluorescence in a population of live cells or in a cell extract of a sample to a negative control provides a means to quantitate gene expression.

In one embodiment of the invention, methods of the invention may employ one or more substrates to detect β-lactamase activity. One example of a suitable substrate for use in methods of the invention is CCF2. CCF2 consists of a cephalosporin core linked to two fluorophores, 7-hydroxycoumarin and fluorescein. In the absence of β-lactamase reporter activity, the substrate molecule remains intact. Excitation of the coumarin at 409 nm results in fluorescence resonance energy transfer (FRET) to the fluorescein moiety. This energy transfer causes the fluorescein to emit a green fluorescence signal with an emission peak of 520 nm. In the presence of β-lactamase reporter activity, the CCF2 substrate is cleaved, disrupting FRET. In this case, excitation of the coumarin at 409 nm results in emission of a blue fluorescence signal with an emission peak of 447 nm. In a population of cells loaded with CCF2 substrate, those that fluoresce blue contain β-lactamase reporter activity while those that fluoresce green do not.

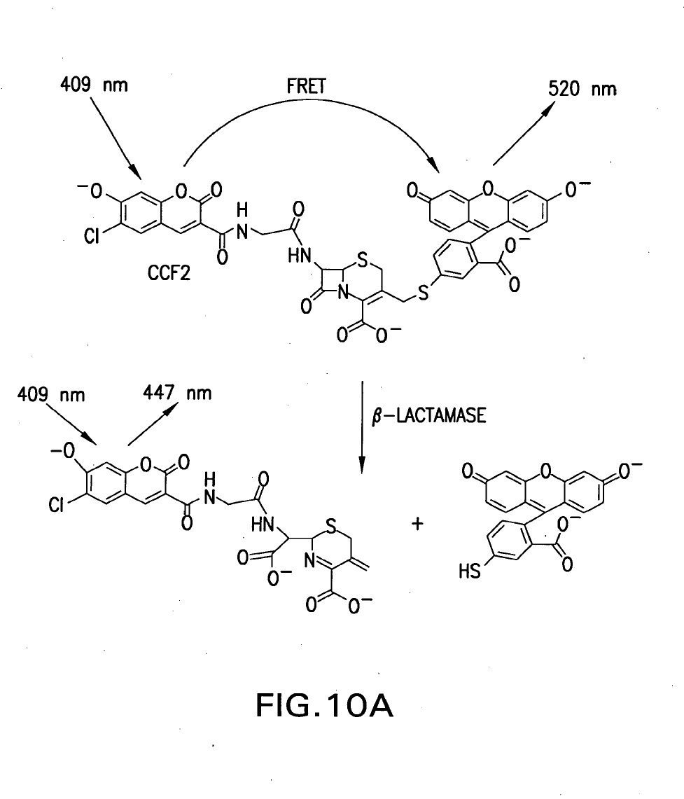

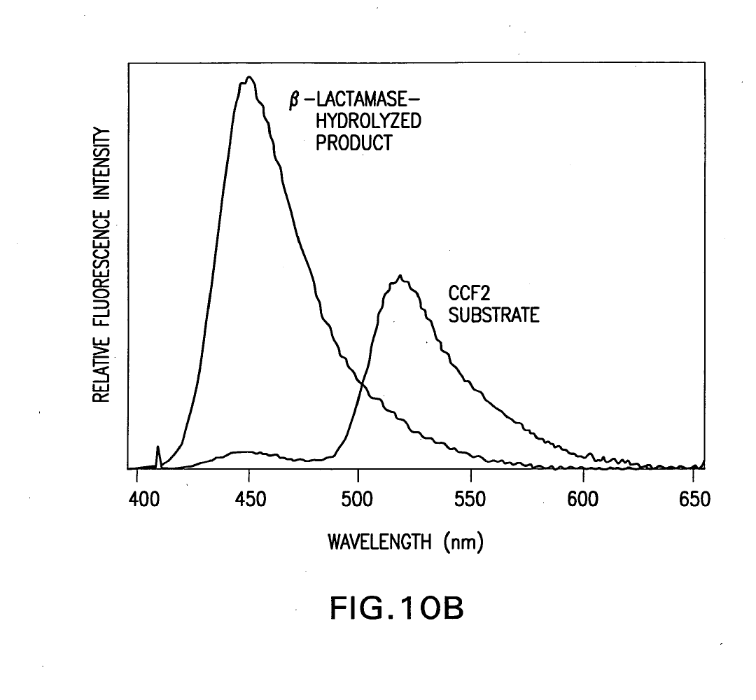

FIG. 10 provides a schematic representation of the hydrolysis of CCF2 by a lactamase. Panel A illustrates how CCF2 is hydrolyzed by β-lactamase and how CCF2 FRET works. Panel B depicts the fluorescence emission spectra of the CCF2 substrate and its hydrolyzed product after excitation at 409 nm.

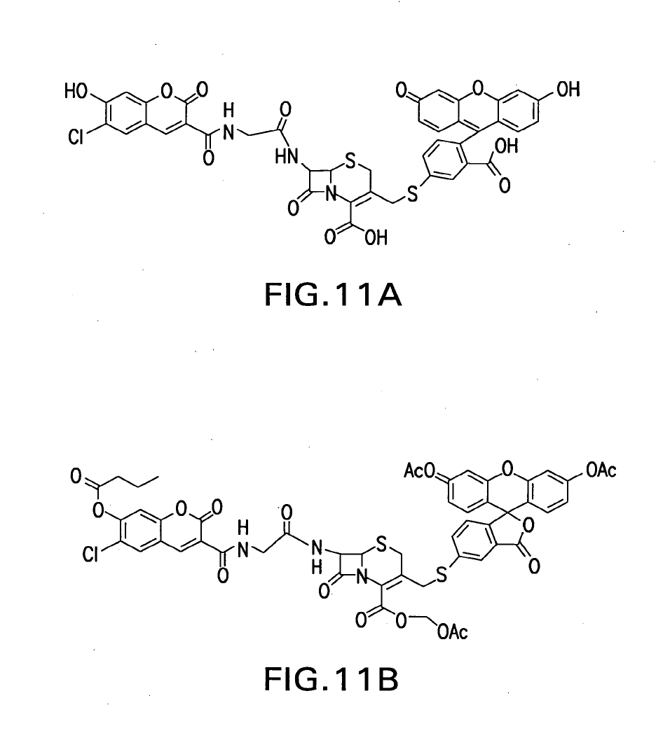

Two derivatives of CCF2 have been developed to enable use of the fluorescent substrate for in vivo or in vitro applications. CCF2-FA may be used for the in vitro detection of β-lactamase activity while CCF2-AM may be used for in vivo detection of β-lactamase activity. CCF2-FA is the free acid form of the CCF2 substrate. This free acid form is water soluble, making it suitable for direct addition to cell lysates. CCF2-AM is a hydrophobic, membrane-permeable, esterified form of the CCF2 substrate. This esterified form is non-toxic, lipophilic and readily enters the cell. Once inside the cell, the CCF2-AM is converted into CCF2. FIG. 11, provides the structures of CCF2-FA (panel A) and CCF2-AM (panel B).

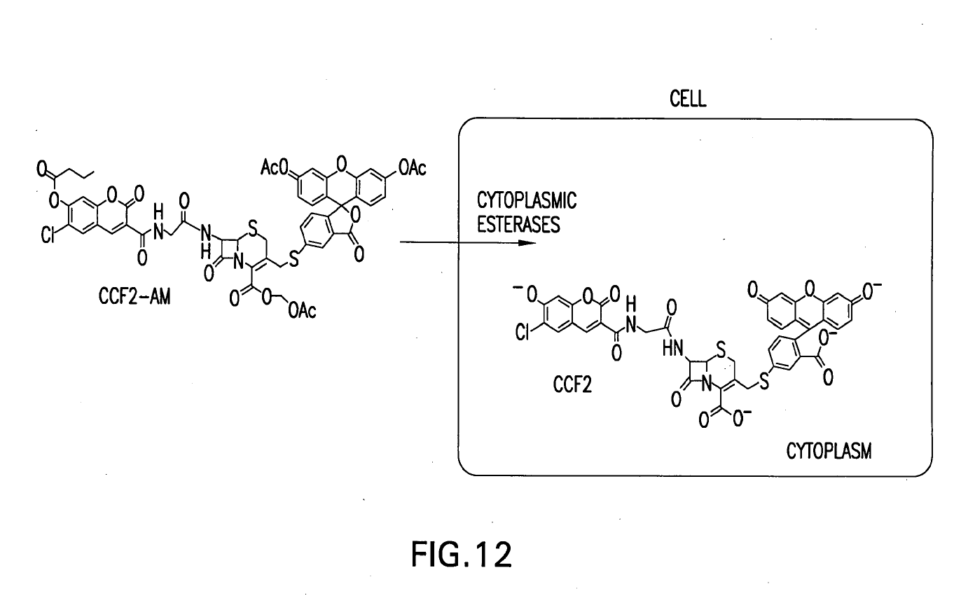

When added to mammalian cells, the lipophilic, esterified CCF2-AM substrate enters the cell via diffusion, where it is cleaved by endogenous cytoplasmic esterases and rapidly converted into its negatively charged form, CCF2 (see FIG. 12). The hydrophilic, charged CCF2 substrate is trapped inside the cell. Over time, this results in cells “loading” with more substrate, thereby increasing the intracellular substrate concentration. This increases the sensitivity of the detection assay without the need for addition of higher concentrations of substrate.

In one aspect, the present invention provides methods of detecting β-lactamase activity in a lysate prepared from a cell. Such methods may entail preparing a cell lysate from cells of interest using a method that preserves the enzymatic activity of β-lactamase, contacting the lysate with a fluorescent substrate and detecting a change in the fluorescence. A stock of fluorescent substrate (e.g., 100 μM CCF2-FA) can be prepared and the appropriate amount of CCF2-FA can be added to the cell lysate. CCF2-FA fluorescence signal may be detected using a fluorescence plate reader or fluorometer.

In another aspect, the present invention provides methods of detecting β-lactamase activity in a living cell. Such methods may entail introducing into the cells a fluorescence substrate for β-lactamase and detecting a change in the fluorescence of the substrate. Detection of the fluorescence signal may be by any means known in the art (e.g., fluorescence microscopy, ratiometric imaging, fluorescence plate reader, FACS).

A nucleic acid molecule of the invention may be prepared and introduced into a host cell as described above. To perform an in vitro assay for β-lactamase activity in a cell lysate, any suitable method of preparing a lysate may be use. A suitable method is one in which the enzymatic activity of β-lactamase is preserved. An example of a suitable protocol is provided below.

Adherent cells may be harvested by dissociating cells with an EDTA-containing buffer using standard methods (e.g. Versene). Cells may then be counted using a cell counter or a hemacytometer and centrifuged. The cell pellet may be washed twice with HBSS or HBS and resuspended in Hank's Balanced Salt Solution (HBSS) or hepes buffered saline (HBS) to a density of 1×10⁷ cells/ml in a microcentrifuge tube. Trypsin-EDTA should not be used to dissociate the cells as over trypsinizing cells may reduce β-lactamase activity by causing cell lysis and proteolysis. For suspension cells, an aliquot may be counted using a cell counter or a hemacytometer. Cells may be harvested by centrifugation and resuspend in HBSS or HBS to a density of 1×10⁷ cells/ml in a microcentrifuge tube.

Cells prepared as described may be frozen in liquid nitrogen or a dry ice/ethanol bath. The tube may then be transferred to a 30° C. water bath until cells are thawed. To prevent degradation, avoid excessive incubation at 30° C. The cells may be frozen and thawed and additional two times making a total of three freeze thaw cycles.

The cells may then be centrifuge the sample in a microcentrifuge at +4° C. at maximum speed to pellet cell debris. The supernatant may be transferred to a sterile microcentrifuge tube. The cell lysate may be stored at −20° C. or at −80° C. Other suitable methods of preparing a cell lysate are known to those skilled in the art. For example, cell lysates can be prepared using sonication or a gentle detergent such as 1% NP-40, 1% IGEPAL CA-630 (Sigma, Catalog no. 1-3021) or 0.5% CHAPS, if desired. For high-throughput applications, cell lysates may be prepared using one of the detergents suggested above. Cells may be lysed directly in the tissue culture well.

A stock solution of 100 μM CCF2-FA may be prepared in Hank's Balanced Salt Solution (HBSS) or HEPES Buffer Saline (HBS). Other phosphate-based buffers such as Phosphate-Buffered Saline (PBS) are also suitable. Aliquot desired volumes into cryovials and freeze quickly by placing the vials on dry ice or in liquid nitrogen. This minimizes freeze/thaw cycles during use. Once the solutions are frozen, transfer the cryovials to a −20° C. freezer. Store the solutions protected from light. When stored under these conditions, the aqueous CCF2-FA stock solution is stable for at least one month.

A suitable protocol for use in a 96 well plate format is as follows. An aliquot of a cell lysate is added to a well of a 96-well plate. To each well containing sample, CCF2-FA stock solution may be added to obtain a final concentration of 10 μM (10-fold-dilution). For example, add 10 μl of CCF2-FA to 901 of cell lysate (total volume=100 μl). Proceed to read the fluorescence signal in a fluorescence plate reader or fluorometer. Although β-lactamase cleaves the CCF2 substrate rapidly, longer incubation times may be required to optimize the fluorescence signal when low levels of the enzyme are present in the cell lysate. For example, the fluorescence signal can be read every 15 minutes for 1 hour.

To detect β-lactamase reporter activity in live cells, the CCF2-AM substrate may be used. CCF2-AM is the membrane-permeable, esterified form of CCF2, and is recommended for in vivo use because it is lipophilic and readily enters the cell. Once cells are “loaded” with CCF2-AM, CCF2 fluorescence signal may be quantified using a variety of methods.

A number of factors can influence the degree of cell loading, and consequently, the success of detection. These factors include: the cell type or cell line used; the density of cells at the time of loading; the temperature at which the cells are loaded; the degree to which the cell line retains the CCF2-AM substrate; and the loading protocol used.

Any suitable cell line may be used in the practice of the methods of the invention. For example, any mammalian cell line or cell type of choice may be used to express a lactamase reporter construct for detection using methods of the invention. This includes cell lines that grow in suspension or as adherent monolayers. Cell lines may vary significantly in their rate and ability to load and retain the CCF2-AM substrate. A suitable general protocol is provided below. One skilled in the art can optimize the protocol far any particular cell line by varying one or more of the factors described above.

Suspension cells are typically loaded at a density of 1-2×10⁶ cells/ml. Adherent cells load with CCF2-AM substrate most efficiently when they are 60-80% confluent at the time of loading. In contrast, confluent cells load poorly. For analysis of gene expression from a stable cell line, cells may be plated such that they will be 60-80% confluent at the time of loading. For transient analysis of gene expression, cells may be transfected using Lipofectamine™ 2000 Reagent available from Invitrogen Corporation, Carlsbad, Calif. (Catalog no. 11668-027) as recommended by the manufacturer (i.e. 90% confluence for 4-6 hours). Cells may then be incubated cells at 37° C. overnight, then trypsinized and re-plated such that the transfected cells are 50-60% confluent. The cells may then be incubated overnight at 37° C., and loaded the next day.

The rate at which cells load with CCF2-AM substrate is affected by temperature. Generally, increasing the temperature (e.g. from room temperature to 37° C.) will increase the loading rate. However, increasing the temperature also increases the rate at which the substrate is exported from the cell, which may result in lower overall steady-state uptake of CCF2-AM. Cells may be loaded at room temperature.

Cells may be loaded for one hour. Cell lines vary in their ability to load and retain the CCF2-AM substrate. For example, lymphoma cells tend to load in 15-30 minutes, while most adherent cells load well in 30 minutes to 1 hour at room temperature. Generally, fluorescence signal is detectable by 15 minutes after loading and increases steadily for about 60 minutes. Longer incubation times may further increase the intensity of the fluorescence signal, but the increase in intensity is smaller than that observed in the first hour. Depending on the cell line and the application, the CCF2-AM loading time can be varied to optimize the fluorescence signal. One skilled in the art can readily optimize loading time using routine experimentation. For example, cell loading may be visualized using a fluorescence microscope (e.g. take a reading every 15 minutes for up to 2 hours) to determining how quickly the cells fluoresce green. Alternatively, loading may be monitored using a bottom-read fluorescence plate reader (e.g. Gemini-EM Fluorescence Microtiter Plate Reader, Molecular Devices or CytoFluor® 4000 Fluorescence Plate Reader, PerSeptive Biosystems).

Two loading protocols are provided below to facilitate cell loading of CCF2-AM, a General Loading Protocol and an Enhanced Loading Protocol. For most cell lines, the General Loading Protocol is recommended and results in efficient cell loading and a highly detectable CCF2-AM fluorescence signal. In some cell lines, using the General Loading Protocol results in a weak fluorescence signal. These cell lines are generally those that possess active anion transport, resulting in export of the substrate (see examples below). For these cell lines, cells may be loaded using the Enhanced Loading Protocol. Depending on the nature of the cell line, the loading protocol can be varied.

Examples of cells that may be loaded using the General Loading Protocol include, but are not limited to, HEK293, COS-7, and Jurkat. Examples of cells that may be loaded using the Enhanced Loading Protocol include, but are not limited to, CHO-K¹, CV-1, ME-180, and HepG2.

Once cells have been loaded with the CCF2-AM substrate, a variety of methods can be used to analyze the fluorescence signal including, but not limited to visual inspection of fluorescent cells using fluorescence microscopy, quantitative analysis of blue and green fluorescence by ratiometric imaging using a fluorescence microscope, quantitative analysis of blue and green fluorescence using a fluorescence plate reader, fluorescence-activated cell sorting (FACS) to isolate cells expressing β-lactamase.

A 1 mM stock solution of CCF2-AM in anhydrous DMSO is called Solution A. Solution A can be stored at −20° C., dessicated and protected from light. When stored under these conditions, Solution A is stable for at least one month. Before each use, let the frozen Solution A warm to room temperature and remove the desired amount of reagent. Immediately recap the vial to reduce moisture uptake and return to −20° C. storage. Once thawed, Solution A may appear slightly yellow. This color change is normal and does not affect the performance of the reagent.

In some embodiments, the present invention provides methods of detecting reporter activity in a cell by loading the cell with a fluorescent substrate and detecting a change in the fluorescence of the substrate. For example, methods of the invention may be used to detect β-lactamase reporter activity in a cell line of interest by loading the cells with the fluorescent CCF2-AM substrate and evaluating the difference in blue and green signal intensity compared to a negative control (cells with no β-lactamase reporter activity). Fluorescent substrate may be loaded into the cells with a 6×CCF2-AM Loading Solution using the General Loading Protocol.

In some embodiments, β-lactamase reporter activity may be detected by introducing a fluorescent substrate for β-lactamase activity into one or more cells, and evaluating blue and/or green fluorescence intensity. For example, the fluorescent CCF2-AM substrate may be introduced into cells and the difference in blue and green signal intensity may be evaluated, for example, compared to a negative control (cells with no β-lactamase reporter activity). In some embodiments, the cells may be adherent cells. In other embodiments, the cells may be suspension cells.

In some methods of the invention, after evaluating blue and/or green fluorescence intensity, the cells may be further cultured. Optionally, cells with a desired activity or activity level (e.g., expressing β-lactamase, expressing β-lactamase at a high level, or not expressing β-lactamase) may be separated from cells not having the desired activity or activity level (e.g., by FACS) and cells having desired activity and/or activity levels may be further cultured. In embodiments of this type, sterility may be maintained throughout the experiment, for example, by performing all manipulations within a tissue-culture hood and preparing solutions using sterile reagents.

In some methods of the invention, a fluorescent substrate may be introduced into one or more cells. For example, 6 μl of Solution A may be added to 54 μl of Solution B (100 mg/ml Pluronic®-F127 surfactant in DMSO and 0.1% acetic acid) and vortexed to mix thoroughly. If Solution B is stored at cooler temperatures, a white precipitate may form or the solution may freeze. Warm and mix the solution at 37° C. until the precipitate dissolves. To the mixture, 940 oil of Solution C (24% (w/v) PEG 400, 18% (v/v) TR40 in water) or 940 μl of HBSS can be added to the combined Solutions A and B (60 μl volume) to obtain a final volume of 1 ml. The combined solutions may be vortexed to mix thoroughly. The combined solutions should be used within two hours of preparation as the substrate degrades over time in aqueous solution. This solution is referred to as 6×CCF2-AM Loading Solution.

Solution C is added to reduce non-specific fluorescence due to substrate that has not entered the cell. The presence of Solution C will interfere with the fluorescence signal if fluorescence signal is to be determined using a top-read fluorescence plate reader or by fluorescence-activated-cell sorting (FACS). In embodiments, using these two techniques, 940 μl of HBSS or PBS (Ca²⁺- and Mg²⁺-free) may be substituted for Solution C. After loading, remove the loading solution and wash the cells with HBSS. Replace with an equal volume of HBSS before taking a reading (if using a fluorescence plate reader) or prepare cells for flow cytometry (if performing FACS). Reading fluorescence signal from a bottom-read fluorescence plate reader provides the best sensitivity.

The following conditions may be used in connection with methods of the invention. When cells to be used are grown in tissue culture plates, any size tissue culture plate may be used (e.g., 96-well format). Tissue culture plates should be selected to be compatible with the detection instrument to be used. When 96-well plates are to be used, black-walled, clear bottom 96-well plates may be used. Adherent cells may be 60-80% confluent at the time of loading and suspension cells may be loaded at a density of 1-2×10⁶ cells/ml. Cells may be loaded at room temperature. Cells may be loaded for varying amounts of time, for example, from about 10 minutes to about 3 hours, from about 20 minutes to about 2 hours, from about 30 minutes to 1.5 hours, or about 1 hour. Cells may be loaded in HBSS or HBS. Cells may also be loaded in serum-containing media, however, CCF2-AM may hydrolyze during prolonged exposure to serum. This may affect the rate of CCF2-AM loading.

For methods involving loading adherent cells, the following protocol for introducing fluorescent substrate into cells may be use. Cells may be plated in any tissue culture format of choice. Methods may include the use of a negative control (no cells), an untransfected control, and/or an uninduced control to determine the background blue and green fluorescence. Methods of the invention may entail removing the growth medium from the cells and adding the appropriate amount of HBSS to each well and adding a solution comprising the fluorescent substrate. Optionally, cells may be washed one or more times with HBBS before adding HBBS and substrate. Typically, the amount of HBBS added to the cells is greater than the amount of solution comprising substrate added. For example, 5 parts HBBS may be added for 1 part solution comprising substrate may be added to the cells. Examples of suitable amounts of solution comprising substrate and HBBS for various tissue culture dishes are as follows: for a 96-well plate 20 μl substrate and 100 μl HBBS; for a 48-well plate 40 μl substrate and 200 μl HBBS; for a 24-well plate 100 μl substrate and 500 μl HBBS; for a 12-well plate 150 μl substrate and 750 μl HBBS; and for a 6-well plate 250 μl substrate and 1250 μl HBBS. The final solution comprising HBBS and substrate may contain from about 100 μM substrate to about 5 mM substrate, from about 250 μM substrate to about 2.5 mM substrate, from about 0.5 mM substrate to about 2 mM substrate, or about 1 mM substrate. After substrate and HBBS have been added, plates may be covered to prevent the solution from evaporating. Plates may be incubated for a suitable time at a suitable temperature. Suitable times are from about 5 minutes to about 5 hours, from about 10 minutes to about 4 hours, from about 20 minutes to about 3 hours, from about 30 to about 2.5 hours, from about 45 minutes to about 2 hours, or about 1 hour. A suitable temperature is one from about 4° C. to about 42° C., from about 10° C. to about 37° C., from about 15° C. to about 30° C., or about room temperature. During incubation, cells may be protected from light. As will be appreciated by those skilled in the art, extending the incubation time may increase the fluorescence signal, but may also increase the background. An optimum time may be determined using routine experimentation. After the cells are loaded, fluorescence may be determined using any technique know in the art. Alternatively, cells may be removed from the loading solution, washed and cultured in any appropriate medium until fluorescence is to be determined.

Methods of the invention may entail introducing a fluorescent substrate into suspension cells. Methods may include the use of a negative control (no cells), an untransfected control, and/or an uninduced control to determine the background blue and green fluorescence. Methods of the invention may entail pelleting a suitable number of cells (e.g., 1-2×10⁵ cells) by centrifugation for each suspension culture to be tested. The pelleted cells may be washed one or more times with a suitable medium, for example, HBSS, and then may be resuspended in a suitable volume of a suitable medium (e.g., 100 μl of HBSS). A solution comprising a fluorescent substrate may be added to the re-suspended cells, for example, to a 100 μl sample, 20 μl of the 6×CCF2-AM Loading Solution may be loaded to obtain a final concentration of 1×. Cells in loading solution may be transferred to a black-walled, clear bottom 96-well tissue culture plate. The plate may be covered to prevent the solution from evaporating. Concentrations of fluorescent substrate, incubation times and temperatures may be the same for suspension cells as those set forth above for adherent cells. During incubation, cells may be protected from the light. An optimum time may be determined using routine experimentation. After the cells are loaded, fluorescence may be determined using any technique know in the art. Alternatively, cells may be removed from the loading solution, washed and cultured in any appropriate medium until fluorescence is to be determined. During the incubation, cells will settle to the bottom of the well. If a bottom-read fluorescence plate reader is to be used to determine fluorescence, the plate should be handled gently as the cells must remain at the bottom of each well for accurate detection to occur. The bottom of the plate should not be touched.

After loading cells (adherent or suspension) with the substrate, cells may be inspected visually (e.g., in a fluorescence microscope) to qualitatively assess the fluorescence signal. If the blue and green fluorescence signal is detectable, the cells may be further processed to quantify the reporter activity and/or to select cells with the desired activity and/or activity level. For example, β-lactamase reporter activity may be quantified in live cells using a suitable technique (e.g., a fluorescence plate reader or ratiometric imaging with a fluorescence microscope). If a fluorescence plate reader is used to detect fluorescence signal in whole cells, note that optimal sensitivity is obtained with a bottom-read fluorescence plate reader. Alternatively, cell lysates can be prepared and used to measure β-lactamase reporter activity using a fluorescence plate reader. In some embodiments, FACS may be used to select cells based on their β-lactamase reporter activity.

As will be appreciated in the art, some cell lines take up fluorescent substrate better than other cell lines. To use methods of the invention with cell lines that take up less substrate than desired, methods of the invention may be modified to enhance substrate uptake. For example, for cells that display weak fluorescence signal (i.e. poor substrate retention) by visual inspection on a fluorescence microscope after being loaded as described above, a different loading solution (e.g., 6×CCF2-AM Enhanced Loading Solution) may be used. Cell lines that typically exhibit an increased fluorescence signal after being loaded with the 6×CCF2-AM Enhanced Loading Solution may be those that possess active ion transport mechanisms including, but not limited to, CHO-K1, CV-1, ME-180, and HepG2.

To enhance substrate uptake, cells may be incubated in a solution comprising a higher concentration of substrate (e.g., CCF2-AM). Solutions may also comprise a non-specific inhibitor of anion transport (e.g., probenecid, see DiVirgilio, et al., (1988) J. Immunol. 140, 915-920). Probenecid (p-[Dipropylsulfamoyl]benzoic acid) is available from Sigma (Catalog no. P-8761). Although the presence of probenecid can increase the amount of substrate retained in the cell, it may be toxic to some cell types. If toxicity is observed upon using loading solutions containing probenecid, omit the probenecid.

A probenecid stock solution may be prepared. For example, a 500 mM stock solution may be prepared by dissolving the appropriate amount of probenecid in 0.5 M NaOH. To prepare a 250 mM stock solution (100×), equal volumes of 500 mM stock solution and 100 mM phosphate buffer pH 8.0 may be mixed and the pH of the resulting 250 mM solution may be adjusted to pH 8.0 with 1 M HCl or 1 M NaOH. Aliquots of the 250 mM probenecid stock solution (100×) may be placed in 1 ml microcentrifuge tubes and stored at −20° C. The solution is stable for at least 4 months.

One suitable solution for enhanced uptake of substrate into cells (e.g., 6×CCF2-AM Enhanced Loading Solution) may be prepared using the following protocol. 12 μl of Solution A may be added to 48 μl of Solution B and vortex. If Solution B is stored at cooler temperatures, a white precipitate may form or the solution may freeze. Warm and mix the solution at 37° C. until the precipitate dissolves. Optionally, 60 μl of probenecid 250 mM stock solution can be added to the combined Solutions A and B (total volume=120 μl). 880 μl of Solution C (940 μl if probenecid is omitted) may be added to the loading buffer to obtain a final volume of 1 ml and vortexed to mix.

Enhanced loading solutions should be used within two hours of preparation as the substrate degrades over time in aqueous solution. Discard any unused solution.

In some embodiments, methods of the invention may entail the use of an enhanced loading solution for the introduction of a fluorescent substrate into a cell. In some embodiments, the cells to be loaded using an enhanced loading solution may be adherent cells. Adherent cells may be plated in any tissue culture format of choice. Methods of the invention may include a negative control (no cells), an uninduced control, and/or an untransfected control to determine the background blue and green fluorescence. Methods of the invention may entail removing the growth medium from the cells and washing the cells once with HBSS. An appropriate amount of HBSS may be added to each well. An appropriate amount of an enhanced loading solution (e.g., 6×CCF2-AM Enhanced Loading Solution in a 6-fold dilution) may be added to the well to obtain a suitable final concentration of substrate (e.g., 2 μM CCF2-AM). The plate may be covered to prevent the solution from evaporating. Incubate the cells at a suitable temperature for a suitable time protected from light. Suitable reagent volumes for tissue culture plate type and times and temperatures of incubation include those set out above for loading adherent cells with a fluorescent substrate. Extending the incubation time may increase the fluorescence signal, but may also increase the background. After incubation, fluorescence signal may be detected using the method of choice. Alternatively, the enhanced loading medium may be removed and replaced with fresh, growth medium (optionally containing 1% probenecid stock) or HBSS (optionally containing 1% probenecid stock) and cultured until fluorescence is detected.

In some embodiments, methods of the invention may comprise the use of an enhanced loading solution to load a fluorescent substrate into a cell. Methods of the invention may include a negative control (no cells), an uninduced control, and/or an untransfected control to determine the background blue and green fluorescence. Methods may comprise pelleting 1-2×10⁵ cells by centrifugation for each suspension culture to be assayed. The cell pellet may be washed once with HBSS, then resuspended in 100 μl of HBSS. 20 μl of an enhanced loading solution (e.g., 6×CCF2-AM Enhanced Loading Solution) may be added to 100 μl of cells in buffer to obtain a final concentration of 1× enhanced loading solution. A 1× enhanced loading solution may comprise a greater concentration of fluorescent substrate than other loading solutions (e.g., 2 μM CCF2-AM). Cells in enhanced loading solution may be transferred to a black-walled, clear bottom 96-well tissue culture plate. The plate may be covered to prevent the solution from evaporating. Suitable times and temperatures of incubation include those set out above for loading adherent cells with a fluorescent substrate. Extending the incubation time may increase the fluorescence signal, but may also increase the background. After incubation, fluorescence signal may be detected using the method of choice. Alternatively, the enhanced loading medium may be removed and replaced with fresh, growth medium (optionally containing 1% probenecid stock) or HBSS (optionally containing 1% probenecid stock) and cultured until fluorescence is detected.

Once the cells (adherent or suspension) have been loaded with fluorescent substrate using any on the methods described herein, the fluorescence signal of the substrate (e.g., CCF2) and its β-lactamase-catalyzed hydrolysis product may be detected in cells using any type of fluorescence microscope with a long-pass dichroic mirror to separate excitation and emission light. The dichroic mirror should be matched to the excitation filter to maximally block the excitation light around 405 nm, yet allow good transmission of the emitted light.

Use of the best filter sets will ensure that the optimal regions of the β-lactamase spectra are excited and passed (emitted). To visually inspect the cells, a long-pass filter passing blue and green fluorescence light may be used so that it is possible to visually identify whether the cells are fluorescing blue or green. Suitable filters sets are commercially available, for example, from Chroma Technologies, Rockingham, Vt. or Omega Optical, Brattleboro, Vt. as specified below. FITC filters should not be used. Most FITC filters block emission of blue light so all cells (even those that contain β-lactamase) will appear green. As will be appreciated by one skilled in the art, when the polypeptide having a detectable activity has a β-lactamase activity and the fluorescent substrate used is CCF2 or a derivative thereof, wild-type cells that do not contain the bla(M) reporter gene and possess no β-lactamase activity will emit a green fluorescence signal, while those that contain the bla(M) reporter gene and are expressing β-lactamase will emit a blue fluorescence signal.

| | | Omega Optical Filter Set |
|---|---|---|
| | Chroma Set #41031 | #XF106-2 for |
| Filters | for β-lactamase | CCF2/GeneBLAzer ™ |
| Excitation filter: | HQ405/20x (405 ± 10) | 400DF15 |
| Dichroic mirror: | 425 DCXR | 420 DCLP |
| Emission filter: | HQ435LP (435 long-pass) | 435ALP |

Methods of the invention may optionally comprise taking photographs of the cells. A color camera that is compatible with the microscope may be used to photograph the cells. Suitable cameras include digital cameras or cameras using a high sensitivity film, such as 400 ASA or greater.

Methods of the invention may comprise monitoring a detectable activity (e.g., β-lactamase activity) in single cells over time. Such methods may comprise the use of microscopic imaging and ratiometric analysis. For methods comprising microscope-based ratiometric analysis, the blue and green fluorescence emissions are analyzed separately by filtering the emitted light through two emission filters, passing either blue or green fluorescence (analogous to using a fluorescence plate reader). By calculating the ratio of blue to green fluorescence intensities, it is possible to numerically analyze β-lactamase activity. To perform ratiometric analysis, a filter set containing separate blue and green emission filters may be used. Suitable filter sets are commercially available from, for example, Chroma Technologies or Omega Optical as specified below.

| | | Omega Optical |
|---|---|---|
| Filters | Chroma Set #71008 | Filter Set #XF124 |
| Excitation filter: | HQ405/20x (405 ± 10) | 400DF15 |
| Dichroic mirror: | 425 DCXR | 415DRLP |
| Emission filter (blue): | HQ460/40m (460 ± 20 nm) | 450DF65 |
| Emission filter (green): | HQ530/30m (530 ± 15 nm) | 535DF35 |

Those skilled in the art will appreciate that, as with other fluorescent dyes, photo-bleaching the dye-loaded cells may be avoided. The CCF2 substrate is particularly sensitive to continuous illumination through a high magnification, high numerical aperture objective with UV or any other wavelength of light that can excite the dye. Continuous excitation of the dye can cause the acceptor fluorophore to be bleached (destroyed) with loss of FRET and appearance of donor fluorescence. This effect is progressive and nonreversible.

To reduce photo-bleaching, limit exposure of cells to excitation light by analyzing fluorescence signal for a few seconds at a time. Alternatively, use a lower magnification objective to reduce exposure of the substrate to light.

In some methods of the invention, detectable activity (e.g., β-lactamase activity) may be detected in cells using a fluorescence plate reader. Methods include, but are not limited to, measuring the fluorescence intensity in cell lysates containing fluorescent substrate (e.g., CCF2-FA); measuring the fluorescence intensity in live cells containing, fluorescent substrate (e.g., CCF2-AM-loaded cells); and/or lysing the fluorescent-substrate-loaded cells (e.g., CCF2-AM-loaded cells) and measuring fluorescence intensity in cell lysates. The last method may provide better sensitivity if using a top-read fluorescence plate reader.

Any fluorescence plate reader may be used to practice one or more of the methods of the invention. In some embodiments, a bottom-read fluorescence plate reader may be used. Such readers are well know in the art and are commercially available (e.g. Gemini-EM Fluorescence Microtiter Plate Reader, Molecular Devices, CytoFluor® 4000 Fluorescence Plate Reader, PerSeptive Biosystems, or Safire Microplate Reader, Tecan). Top-read fluorescence plate readers (e.g. Gemini-XS Fluorescence Microtiter Plate Reader, Molecular Devices) can be used, however, lower sensitivity may be observed and extra manipulation steps are required before fluorescence signal can be measured in live cells.

Use the optimal filter set to detect ratiometric blue and green readout. Filter sets are included with some fluorescence plate readers, while others require that filters be obtained separately. Filters may be obtained separately, for example, from Chroma Technologies. One suitable filter set is

Chroma Set #APR1

| | |
|---|---|
| Excitation filter: | HQ405/20x (405 ± 10 nm) |
| Emission filter (blue): | HQ460/40m (460 ± 20 nm) |
| Emission filter (green): | HQ530/30m (530 ± 15 nm) |

In the practice of methods of the invention, cells may be plated in any size tissue culture format of choice. One skilled in the art will appreciate the necessity of ensuring that the fluorescence plate reader to be used can accommodate the plate format selected.

In methods that comprise assaying for β-lactamase activity in a 96-well format, cells may be plated in a black-walled, clear-bottom microplate with low autofluorescence (Costar, Catalog no. #3603). Using a black-walled microplate blocks any signal from adjoining wells during reading. For larger-sized tissue culture formats, use of clear tissue culture plates is acceptable.

Those skilled in the art are aware that some plates/plate readers exhibit edge effects that may affect data. If edge effects are noticed, consider the plate layout when setting up the assay.

The bottom of the microtiter plate should not be touched nor should dust be allowed to cover the tissue culture surface. Fingerprints and dust can autofluoresce, introducing well-to-well variability in replicate wells.

Methods of the invention will typically include negative controls (e.g., loading buffer with no cells, cells with no β-lactamase activity, etc.) to determine the background blue and green fluorescence.

Methods of the invention may be practiced using a top-read fluorescence plate reader. In methods involving the quantitating of a fluorescent substrate (e.g., CCF2-AM) fluorescence signal in live cells using a top-read fluorescence plate reader, the dyes from Solution C in the 6×CCF2-AM Loading Solution will interfere with the fluorescence signal. In addition, some components of cell culture media may also interfere with the fluorescence signal. Thus, in methods of this type, the loading solution (e.g., the 6×CCF2-AM Loading Solution) and any cell culture media should be removed from the cells prior to determining fluorescence. For example, cells may be loaded as described above. After loading, the loading solution may be removed and the cells may be washed (e.g., with HBSS). An appropriate amount of HBSS may then be added to the well and the fluorescence signal determined using the top-read fluorescence plate reader. In some embodiments, for example, those in which the cells are not going to be cultured after fluorescence determination, cells may be lysed and then the fluorescence signal determined in the cell lysate as described above. In embodiments where the cells are to be cultured after determination of the fluorescence signal, the HBSS may be removed from the cells and replaced with an appropriate amount of fresh, complete growth media. The cells may then be incubated under appropriate conditions.

In some methods of the invention, it may be desirable to calculate a ratio of blue and green fluorescence intensities. Such a ratio may be calculated by dividing the 460 m emission (blue channel) reading by the 530 nm emission (green channel) reading. Background fluorescence obtained at each wavelength may be subtracted from the observed emission before the ratio is calculated. Background may be determined by reading one or more of the negative controls (e.g., no cells). Thus, a ratio may be calculated as follows:

$$Ratio = \frac{( signal at 460 nm - background at 460 nm )}{( signal at 530 nm - background at 530 nm )}$$

The ratio obtained from experimental samples may be compared to the ratio obtained from the appropriate negative controls. One skilled in the art will appreciate that background values are highly dependent on instrument specific factors and on the length of time the lamp in the instrument has been lit. Thus, methods of the invention may comprise determining a background value on each read.

In some methods of the invention, cells may be sorted by FACS after loading with a fluorescent substrate. Any flow cytometer may be used to detect fluorescent-substrate-loaded cells (e.g., CCF2-AM-loaded cells) by flow cytometry. A Krypton laser with violet excitation (407 nm, 413 nm, or multiline violet 407-415 nm) at 60 mW may be used in practice of methods of this type. The flow cytometer may be equipped with the proper optical filters to detect the fluorescence signal from the substrate. When the substrate is CCF2, the fluorescence signal may be detected using HQ460/50m (blue) and HQ535/40m (green) bandpass filters separated by a 490 nm dichroic mirror. Selection of other filter sets suitable for the detection of signals from other fluorescent substrates may be accomplished by one skilled in the art using routine experimentation.

Methods of the invention may comprise aligning and optimizing the instrument to bemused. Methods may also entail running a negative control sample (e.g., untransfected or uninduced cells) and a positive control sample to adjust PMT levels and compensation values for optimal separation of the blue and green fluorescence signals. A suitable positive control may be cells expressing the activity to be assayed loaded with a suitable substrate (e.g., cells expressing β-lactamase loaded with CCF2-AM). Other condition for determining fluorescence and sorting cells expressing the desired activity and/or level of activity can be determined by those skilled in the art using routine experimentation.

In methods of the invention that involve sorting of the cells after loading, cells may be loaded as described above except that the loading solution should not contain Solution C. Instead of Solution C the loading buffer may comprise any suitable buffer or medium that does not interfere with the fluorescence detection, for example, HBSS. Cells to be sorted according to the methods of the invention may be suspended in a sorting buffer. Suitable sorting buffers include calcium- and magnesium-free HBSS (Invitrogen Corporation, Carlsbad, Calif., Catalog no. 14175-095) containing 25 mM HEPES (pH 7.3) and 0.1% BSA. In some embodiments, cells to be sorted may be suspended in serum-free medium buffered with 25 mM HEPES (pH 7.3) and 0.1% BSA. This may be useful if cells do not remain sufficiently viable other sorting buffers. Typically, cells are not sorted in tissue culture medium as the buffering capacity is weak and can cause the sample pH to increase in air.

When methods of the invention involve sorting adherent cells, after loading, cells may be removed cells from the tissue culture surface and washed once with a suitable sorting buffer (e.g., calcium- and magnesium-free HBSS). Cells may then be resuspend in sorting buffer at a density of 3-5×10⁶ cells/ml. Cells may be in a single cell suspension.

When methods of the invention involve sorting suspension cells, the cells may be loaded as described above. After loading, cells may be washed with a suitable sorting buffer (e.g., calcium- and magnesium-free HBSS), and resuspended in sorting buffer at a density of 5−10×10⁶ cells/ml.

In methods of the invention that entail cell sorting, cells should be in a single cell suspension during sorting. Formation of aggregates (a major problem with adherent cells) can result in subobtimal sorting due to clogging of the flow cytometer and potential contamination of the sample with unwanted cells. Thus methods of the invention may entail preventing aggregation of cells. Cell aggregation may be prevented by removing divalent metal ions from solutions. Cell aggregation may be prevented by performing all washes with Ca²⁺- and Mg²⁺-free solutions, and/or resuspending cells in Ca²⁺- and Mg²⁺-free buffers. When methods of the invention involve the use of serum-containing solutions (e.g., if adding serum is added to the cell suspension to preserve cell viability), methods of the invention may entail dialyzing the serum before use to remove Ca²⁺ and other divalent cations.

Methods of the invention may be optimized using routine experimentation for use with cell types and fluorescent substrates known to those skilled in the art. Factors that may be considered during optimization of the methods disclosed herein may vary with the initial results observed.

In some initial in vitro experiments, a weak fluorescence signal may be observed. This may be the result of low β-lactamase expression. One skilled in the art may consider one or more of the following to optimize the reaction conditions: i) increasing the incubation time of the cell lysate with the fluorescent substrate (e.g., CCF2-FA); ii) re-assessing transfection conditions; and iii) using a different transfection reagent (e.g., Lipofectamine™ 2000 Invitrogen Corporation, Carlsbad, Calif.). A weak fluorescence signal may also result from adherent cells that were dissociated using trypsin-EDTA when preparing a lysate. One skilled in the art may consider using a different dissociation method as over-trypsinizing cells may affect fluorescence signal by causing cell lysis and proteolysis. Versene may be use to dissociate cells. Weak fluorescence signal may also be caused by inefficient cell loading. One skilled in the art might consider in vitro detection of activity. For in vitro detection of β-lactamase, CCF2-FA may be used.

In some initial in vitro experiments, no fluorescence signal may be observed. This may be a result of degradation of the fluorescent substrate. For example, CCF2-FA substrate or stock solution may have been exposed to light during storage or may not have been stored at −20° C. One skilled in the art can readily optimize storage conditions of the substrate, for example, by storing CCF2-FA stock solutions protected from light and at −20° C. Another factor is the method used to prepare the cell lysate, which may have been prepared using a method that destroys the activity of the β-lactamase enzyme. One skilled in the art can adjust the methods used to prepare cell lysates to preserve the activity of the β-lactamase enzyme.

In some initial in vitro experiments, well-to-well variability in replicate wells (most notable when using top-read fluorescence plate readers) may be observed. This may occur if bubbles are present in the cell lysates and may be avoided by carefully transferring cell lysates to a new tissue culture plate, taking care not to introduce bubbles. Variability may also be caused if the bottom of the microtiter plate is touched. The bottom of the microtiter plate should not be touched as fingerprints can autofluoresce. Variabilty may also be caused when the microtiter plate is covered with dust or lint. Since dust can autofluoresce the bottom and top surface of the microtiter plate should be kept free of dust.

In some initial in vivo experiments, all cells may fluoresce green. This may be caused by poor transfection efficiency. One skilled in the art may consider re-assess transfection conditions and/or using a different transfection reagent (e.g., Lipofectamine™ 2000 Invitrogen Corporation, Carlsbad, Calif.). All cell may fluoresce green if a FITC filter set or other improper filter set is used. One skilled in the art can select a suitable filter set, for example, a filter set that allows both blue (460 nm) and green (520 mm) visualization.

In some initial in vivo experiments, a weak fluorescence signal may be observed. This may be caused by poor substrate retention and corrected by using the enhanced loading methods described herein. Weak fluorescence may be observed if the cells are too dense and may be corrected by plating cells such that they will be 60-80% confluent at the time of loading. Weak fluorescence may be caused by low β-lactamase expression. One skilled in the art might consider i) increasing cell loading time; ii) using the enhanced loading methods described herein; or iii) re-assessing transfection conditions. Weak fluorescence can be caused by loading cell at 37° C. and can be corrected by adjusting the loading temperature (e.g., loading cells at room temperature) Weak fluorescence may be observed if cells were loaded in serum-containing media and may be corrected by loading cells in HBSS or HBS. Weak fluorescence may also be observed if a top-read fluorescence plate reader is used in the presence of media or Solution C and can be corrected by omitting these components or washing the cells to remove them prior to reading.

In some initial in vivo experiments, a hazy background or difficulty visualizing fluorescing cells under the microscope may be observed. This may be caused if cells loaded in the absence of Solution C and may be corrected by adding Solution C to the 6×CCF2-AM Loading Solution.

In some initial in vivo experiments, no fluorescence signal is observed. This may occur if the loading solution is degraded. The loading solution should be used with two hours of making it. This may also be caused by degradation of the fluorescent substrate and corrected by proper storage of the substrate. For example, Solution A should be stored at −20° C., dessicated and protected from light.

In some initial in vivo experiments, cells may detach (in sheets) from the surface of the well. This may be a result of the cell line not being an adherent cell line and may be corrected by plating cells on Matrigel-treated wells. This may also be caused by the cells being sensitive to the surfactant (e.g., from Solution B in the 6×CCF2-AM Loading Solution) and may be corrected by reducing the loading time (e.g. 30 to 45 minutes).

In some initial in vivo experiments, cells exhibit toxicity when loaded using the enhanced loading methods described herein. This may be caused by the probenecid is present in the loading solution and may be corrected by preparing the enhanced loading solution without probenecid and/or loading cells for less time (e.g. 30 to 45 minutes).

### Example 3

In some embodiments, the present invention provides nucleic acid molecules comprising a nucleic acid sequence encoding a polypeptide having a detectable activity. Such nucleic acid molecules may also comprise one or more of features including, but not limited to, recombination sites. One non-limiting example of a nucleic acid molecule of the invention is pcDNA™ 6.2/GeneBLAzer™-DEST. Nucleic acid molecules of the invention may be used to facilitate in vivo or in vitro detection of β-lactamase reporter activity in cells (e.g., mammalian cells) using a fluorescent substrate. Methods of the invention provide a highly sensitive and accurate method to quantitate gene expression in cells (e.g., mammalian cells).

According to one aspect, nucleic acid molecules of the invention may comprise one or more of the following features: one or more promoters (e.g., human cytomegalovirus immediate-early (CMV) promoter/enhancer for high-level expression in a wide range of mammalian cells, SV40 early promoter, etc.); one or more nucleic acid sequence encoding a polypeptide having a detectable activity (e.g., a nucleic acid sequence encoding β-lactamase bla(M) reporter gene for C-terminal (pcDNA™ 6.2/cGeneBLAzer™-DEST) or N-terminal (pcDNA76.2/nGeneBLAzer™-DEST) fusion to the gene of interest); one or more recombination sites (e.g., attR1 and attR2, downstream of the CMV promoter for recombinational cloning of the gene of interest from an entry clone); one or more selectable markers (e.g., the chloramphenicol resistance gene, the blasticidin resistance gene, the spectinomycin resistance gene, the ampicillin resistance gene, any one or more of which may be located between the recombination sites (e.g., attR sites) for counterselection); one or more negative selection markers (e.g., the ccdB gene, which may be located between the two attR sites for negative selection); one or more tag sequences (e.g., the V5 epitope tag for detection using Anti-V5 antibodies); one or more polyadenylation signals (e.g., the Herpes Simplex Virus thymidine kinase polyadenylation signal for proper termination and processing of the recombinant transcript); one or more sequences that permit recovery of single strands (e.g., the f1 intergenic region for production of single-strand DNA in F plasmid-containing E. coli); and one or more origin of replication (e.g., the pUC origin, the SV40 early promoter and origin for expression, which may permit stable propagation of the plasmid in mammalian hosts expressing the SV40 large T antigen, etc.).

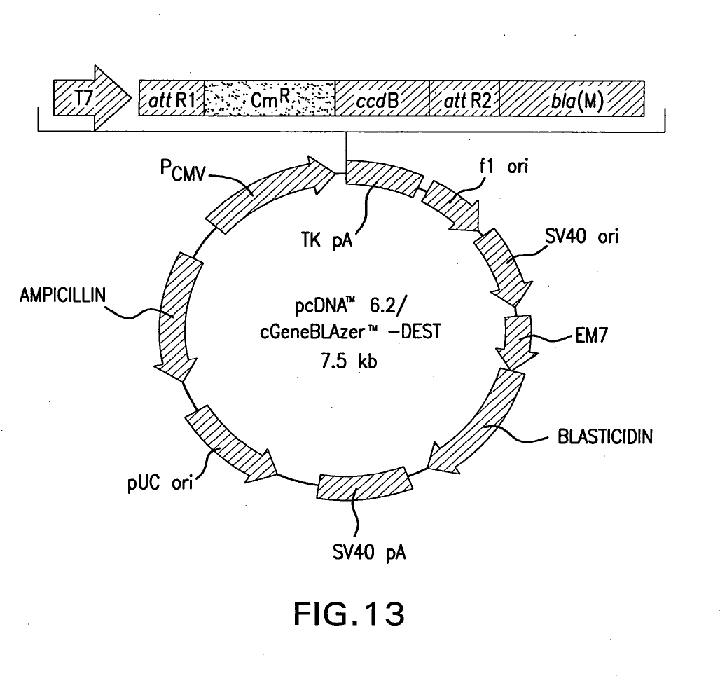

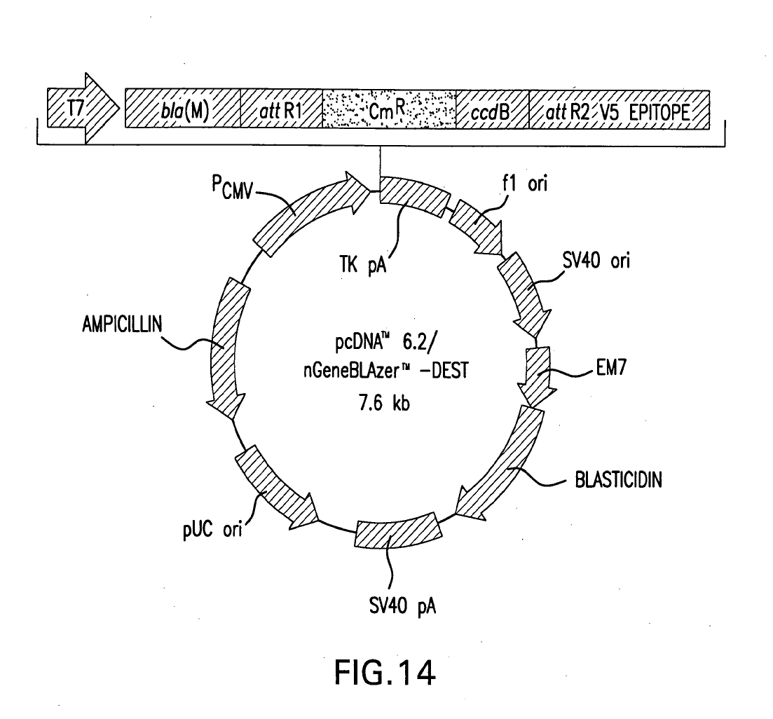

A map of pcDNA™ 6.2/cGeneBLAzer™-DEST and its DNA sequence are provided as FIG. 13 and Table 32, and a map and sequence of pcDNA™ 6.2/nGeneBLAzer™-DEST are provided as FIG. 14 and Table 33, respectively.

In general, methods of the invention may comprise inserting a sequence of interest into a first nucleic acid molecule of the invention, performing one or more recombination reactions with at least a second nucleic acid molecule of the invention to produce a third nucleic acid molecule of the invention and introducing the third nucleic acid molecule of the invention into one or more host cells. Methods may also include selecting a cell that comprises a nucleic acid molecule of the invention (e.g., a stable cell line). Suitable recombination sites are known to those skilled in the art. Nucleic acid molecules comprising such recombination sites and recombination proteins capable of causing recombination between such sites are commercially available, for example, from Invitrogen Corporation, Carlsbad, Calif. under the trade name of GATEWAY®. The GATEWAY® Technology manual is specifically incorporated herein by reference.

Methods of the invention may permit the detection and quantification of gene expression in cells (e.g., mammalian cells). Materials and methods of the invention are suitable for use as a sensitive reporter of gene expression in living mammalian cells using fluorescence microscopy. Materials and methods of the invention provide a ratiometric readout to minimize differences due to variability in cell number, substrate concentration, light intensity, and emission sensitivity. Materials and methods of the invention are compatible with a wide variety of in vivo and in vitro applications including microplate-based transcriptional assays and flow cytometry. Materials and methods of the invention provide a flexible and simple assay development platform for gene expression in cells (e.g., mammalian cells). Materials and methods of the invention typically use a non-toxic substrate that allows continued cell culturing after quantitation analysis.

To join a nucleic acid sequence encoding a polypeptide of interest with a nucleic acid sequence encoding a polypeptide having a detectable activity, a nucleic acid molecule may be constructed in which a sequence encoding a polypeptide of interest is located between two recombination sites that do not recombine with each other. Examples of suitable nucleic acid molecules includes entry vectors available from Invitrogen Corporation, Carlsbad, Calif. Many entry vectors including pENTR/D-TOPO® (Catalog no. K2400-20) are available from Invitrogen to facilitate generation of entry clones.

In some methods of the invention, a fusion protein may be produced. Fusion proteins may be constructed such that one or more stop codons are present in the nucleotide sequence encoding the fusion protein. In some embodiments of the invention such stop codons may be suppressed, for example, by providing a suppressor tRNA that recognizes one or more of the stop codons. Systems to provide such suppressor tRNAs are commercially available, for example, the Tag-On-Demand™ System which allows expression of both native and C-terminally-tagged recombinant protein from the same expression construct is commercially available from Invitrogen Corporation, Carlsbad, Calif.

The Tag-On-Demand™ System is based on stop suppression technology originally developed by RajBhandary and colleagues (see Capone, et al. (1985) EMBO J. 4, 213-221) and comprises a recombinant adenovirus expressing a tRNA^{ser} suppressor. When an expression vector encoding a gene of interest with the TAG (amber stop) codon is transfected into mammalian cells, the stop codon will be translated as serine, allowing translation to continue and resulting in production of a C-terminally-tagged fusion protein. For more information, refer to The Tag-On-Demand™ Suppressor Supernatant manual, which is specifically incorporated herein by reference.

In some embodiments, it may be desirable to express a human or mouse gene of interest. Nucleic acid molecules comprising a nucleic acid sequence encoding various human or mouse polypeptides are commercially available, for example, Ultimate™ Human ORF (hORF) or Ultimate™ Mouse ORF (mORF) Clones are available from Invitrogen Corporation, Carlsbad, Calif. Such clones may be fully-sequenced clones provided in a GATEWAY® entry vector that is ready-to-use in an LR recombination reaction with a pcDNA™ 6.2/GeneBLAzer™-DEST vector. In addition, each clone contains a TAG stop codon, making it fully compatible for use in the Tag-On-Demand™ System.

In some embodiments, methods of the invention may entail expressing one or more polypeptides of interest from one or more nucleic acid molecules of the invention (e.g., from pcDNA™ 6.2/cGeneBLAzer™-DEST). A nucleic acid sequence encoding the polypeptide of interest will typically contain a Kozak translation initiation sequence with an ATG initiation codon for proper initiation of translation (see, Kozak, M. (1987) Nucleic Acids Res. 15, 8125-8148, Kozak, M. (1991) J. Cell Biology 115, 887-903, and Kozak, M. (1990) Proc. Natl. Acad. Sci. USA 87, 8301-8305). An example of a Kozak consensus sequence is provided below. The ATG initiation codon is shown underlined.

(G/A)NNATGG

Other sequences are possible, but the G or A at position −3 and the G at position +4 are the most critical for function (shown in bold).

Nucleic acid molecules of the invention (e.g., pcDNA™6.2/cGeneBLAzer™-DEST) may allow expression of recombinant proteins containing a C-terminal β-lactamase reporter. In some embodiments, nucleic acid molecules of the invention may be used to express both a native and a C-terminal fusion protein from the same construct (e.g., by suppression of a stop codon to produce the fusion protein). In embodiments were it is desired to include the β-lactamase reporter fused to a polypeptide of interest, the nucleic acid sequence encoding the polypeptide of interest should contain a Kozak initiation sequence, should not contain a stop codon, and should be in frame with the bla(M) reporter gene after recombination. In embodiments where it is desired to express a native and a C-terminal-fused polypeptide from the same construct (e.g., by suppressing a stop codon), the nucleic acid sequence encoding the polypeptide of interest should contain a Kozak initiation sequence, should contain a stop codon (e.g., TAG), and should be in frame with the bla(M) reporter gene after recombination.

In some embodiments of the invention, materials and methods of the invention may be used to produce a fusion protein in which a polypeptide of interest is fused with a β-lactamase reporter on the N-terminus. Such fusion polypepes may also comprise a tag sequence on the C-terminus. For example, pcDNA™ 6.2/nGeneBLAzer™-DEST allows expression of recombinant proteins containing an N-terminal β-lactamase reporter and a C-terminal V5 epitope tag, if desired, and contains an ATG initiation codon within the context of a Kozak consensus sequence. This vector may be used in conjunction with the Tag-On-Demand™ System. In embodiments where it is desired to include the β-lactamase reporter, the nucleic acid sequence encoding a polypeptide of interest should not contain a Kozak initiation sequence and should be in frame with the bla(M) reporter gene after recombination. In embodiments where it is desired to include the V5 epitope tag, the sequence encoding a polypeptide of interest should not contain a stop codon and should be in frame with the V5 epitope after recombination. In embodiments where it is desired to include the V5 epitope for use with a suppressor tRNA (e.g., in the Tag-On-Demand™ System), the nucleic acid sequence encoding the polypeptide of interest should contain a stop codon recognized by the suppressor tRNA (e.g., TAG for Tag-On-Demand™), and should be in frame with the V5 epitope after recombination. In embodiments where it is desired to express an N-terminal fusion protein with a native C-terminus, for example to not include the V5 epitope tag when using pcDNA™6.2/nGeneBLAzer™-DEST, the sequence encoding a polypeptide of interest should contain a stop codon.

In general, methods of the invention may comprise performing an LR recombination reaction using the attL-containing entry clone and the attR-containing pcDNA™ 6.2/GeneBLAzer™-DEST vector; transform the reaction mixture into a suitable E. coli host; and selecting for expression clones.

Some of the nucleic acid molecules of the invention may comprise one or more selectable markers that permit selection against hosts comprising nucleic acid molecules containing the marker. For example, pcDNA™ 6.2/GeneBLAzer™-DEST vectors contain the ccdB gene. These vectors can be propagated using Library Efficiency® DB3.1 Competent Cells (Invitrogen Corporation, Carlsbad, Calif., Catalog no. 11782-018). The DB3.1 E. coli strain is resistant to CcdB effects and can support the propagation of plasmids containing the ccdb gene. General E. coli cloning strains including TOP10 or DH5α can not be used for propagation and maintenance of the pcDNA™6.2/GeneBLAzer™-DEST vectors as these strains a sensitive to CcdB effects.

The recombination region of the expression clone resulting from pcDNA™ 6.2/cGeneBLAzer™-DEST×entry clone is shown in FIG. 15. Shaded regions correspond to DNA sequences transferred from the entry clone into pcDNA™ 6.2/cGeneBLAzer™-DEST by recombination. Non-shaded regions are derived from the pcDNA™ 6.2/cGeneBLAzer™-DEST vector. Bases 922 and 2605 of the pcDNA™ 6.2/cGeneBLAzer™-DEST vector sequence are marked.

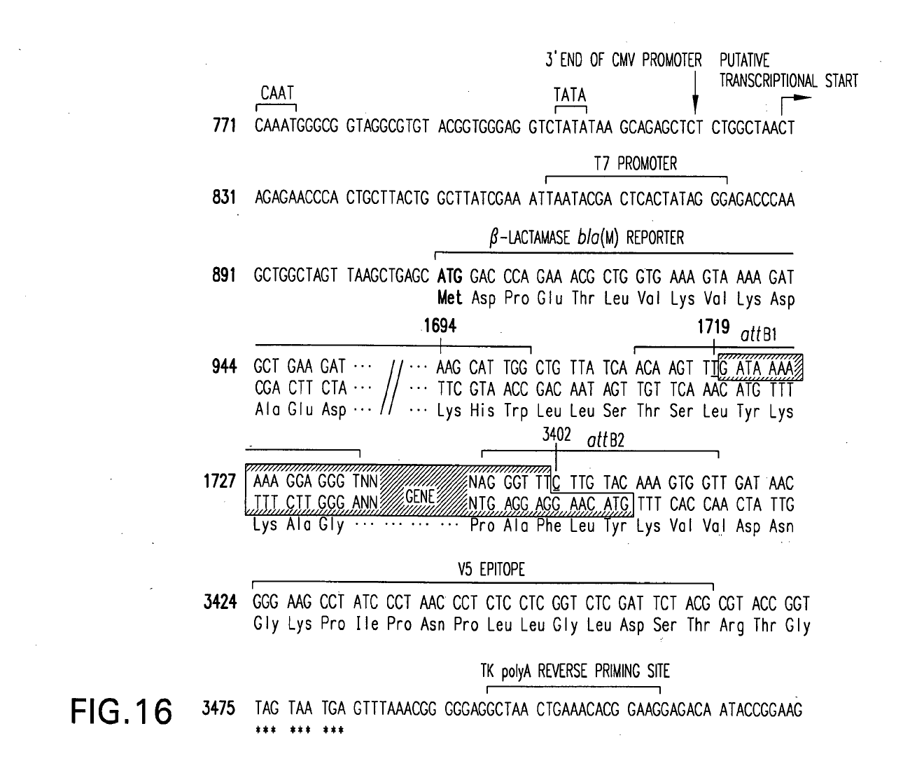

The recombination region of the expression clone resulting from pcDNA™ 6.2/nGeneBLAzer™-DEST×entry clone is shown in FIG. 16. Shaded regions correspond to DNA sequences transferred from the entry clone into pcDNA™ 6.2/nGeneBLAzer™-DEST by recombination. Non-shaded regions are derived from the pcDNA™ 6.2/nGeneBLAzer™-DEST vector. Bases 1719 and 3402 of the pcDNA™ 6.2/nGeneBLAzer™-DEST vector sequence are marked.

A nucleic acid molecule containing a nucleic acid sequence encoding a polypeptide of interest located between to recombination sites, (e.g., an entry clone containing a gene of interest between two attR sites), a recombination reaction can be performed (e.g., an LR reaction) between the entry clone and the pcDNA™ 6.2/GeneBLAzer™-DEST vector, and the reaction mixture can be transformed into a suitable E. coli host to select for an expression clone. A positive control may be included in the experiment, such as pENTR™-gus positive control supplied with the LR CLONASE™ enzyme mix available from Invitrogen Corporation, Carlsbad, Calif. Any recA, endA E. coli strain including TOP10, DH5α™, or equivalent can be used for transformation. Do not transform the LR reaction mixture into E. coli strains that contain the F′ episome (e.g. TOP10F′). These strains contain the ccdA gene and will prevent negative selection with the ccdb gene.

Some nucleic acid molecules of the invention may contain the EM7 promoter and the Blasticidin resistance gene (e.g., pcDNA™ 6.2/GeneBLAzer™-DEST vectors). The blasticidin resistance gene allows for selection of E. coli transformants using Blasticidin. For selection, use Low Salt LB agar plates containing 100 μg/ml Blasticidin. For Blasticidin to be active, the salt concentration of the medium must remain low (<90 mM) and the pH must be 7.0. Blasticidin is commercially available, for example, from Invitrogen Corporation, Carlsbad, Calif.

In some embodiments, methods of the invention may be practiced using one or more of the following materials: purified plasmid DNA of an entry clone (50-150 ng/l in TE, pH 8.0); pcDNA™ 6.2/cGeneBLAzer™-DEST or pcDNA™6.2/nGeneBLAzer™-DEST vector (150 ng/μl in TE, pH 8.0); LR CLONASE™ enzyme mix (Invitrogen Corporation, Carlsbad, Calif., Catalog no. 11791-019; keep at −80° C. until immediately before use); 5×LR CLONASE™ Reaction Buffer (supplied with the LR CLONASE™ enzyme mix); pENTR™-gus positive control, optional (50 ng/μl in TE, pH 8.0; supplied with the LR CLONASE™ enzyme mix); TE Buffer, pH 8.0 (10 mM Tris-HCl, pH 8.0, 1 mM EDTA); 2 μg/μl Proteinase K solution (supplied with the LR CLONASE™ enzyme mix; thaw and keep on ice until use); appropriate competent E. coli host and growth media for expression; S.O.C. Medium; and LB agar plates containing the appropriate antibiotic to select for expression clones.

One suitable protocol for carrying out methods of the invention may entail adding the following components to 1.5 ml microcentrifuge tubes at room temperature and mixing.

| Component | Sample | | Positive Control |
|---|---|---|---|
| Entry clone (100-300 ng/reaction) | 1-10 | μl | — |
| Destination vector (150 ng/μl) | 2 | μl | 2 μl |
| pENTR ™-gus (50 ng/μl) | — | | 2 μl |
| 5X LR CLONASE ™ Reaction Buffer | 4 | μl | 4 μl |
| TE Buffer, pH 8.0 | to 16 | μl | 8 μl |

To include a negative control, a second sample reaction may be prepared omitting the LR CLONASE™ enzyme mix.

Methods of the invention may entail removing the LR CLONASE™ enzyme mix from −80° C. and thawing on ice (˜2 minutes); vortexing the LR CLONASE™ enzyme mix briefly twice (2 seconds each time); adding 4 μl of LR CLONASE™ enzyme mix to each sample and mixing well by pipetting up and down; incubating reactions at 25° C. for 1 hour (extending the incubation time to 18 hours typically yields more colonies); adding 2 μl of the Proteinase K solution to each reaction; incubating for 10 minutes at 37° C.; transforming 1 μl of the LR recombination reaction into a suitable E. coli host (follow the manufacturer's instructions); and selecting for expression clones. The LR reaction may be stored at −20° C. for up to 1 week before transformation, if desired.

Typically, if E. coli cells with a transformation efficiency of 1×10⁸ cfu/μg are used, the LR reaction should give >5000 colonies if the entire transformation is plated.

The ccdB gene mutates at a very low frequency, resulting in a very low number of false positives. True expression clones will be ampicillin-resistant and chloramphenicol-sensitive. Transformants containing a plasmid with a mutated ccdB gene will be both ampicillin- and chloramphenicol-resistant. To check your putative expression clone, test for growth on LB plates containing 30 pg/ml chloramphenicol. A true expression clone will not grow in the presence of chloramphenicol.

In some embodiments, a nucleic acid molecule comprising a nucleic acid sequence encoding a polypeptide of interest fused to a polypeptide having a detectable activity may be sequenced to ensure that the coding regions of the polypeptide of interest and the polypeptide having a detectable activity are in the same reading frame. For example, to confirm that sequence encoding a polypeptide of interest is in frame with the bla(M) reporter gene, the expression construct may be sequenced. Sequencing primers may be designed such that a forward primer hybridizes within the 3′ end of the sequence encoding a polypeptide of interest to sequence through the attB2 site and the 5′ region of the bla(M) reporter gene for a C-terminal fusion. A reverse primer that hybridizes within the bla(M) reporter gene cannot be used as any primer that hybridizes within the bla(M) reporter gene will also hybridize within the ampicillin resistance gene on the plasmid, contaminating the results. Thus, only the sense strand of an expression construct can be sequenced. The T7 Promoter primer may be used to sequence through the attB1 site and into the 5′ region of the sequence encoding a polypeptide of interest. Refer to FIG. 15 for the location of the T7 Promoter primer binding site.

N-terminal fusion proteins prepared according to one aspect of the invention can be sequenced to confirm that the sequence encoding a polypeptide of interest is in frame with the sequence encoding the bla(M) reporter gene or the V5 epitope tag. Sequencing primers may be designed such that a reverse primer hybridizes within the 5′ end of the sequence encoding the polypeptide of interest to sequence through the attB1 site and the 3′ region of the sequence encoding the bla(M) reporter gene. Forward primers that hybridize within the bla(M) reporter gene cannot be used as any primer that hybridizes within the α-lactamase reporter gene will also hybridize within the ampicillin resistance gene, contaminating the results. Thus, only the anti-sense strand of the expression construct can be sequenced. The TK polyA Reverse primer to sequence can be used to sequence through the attB2 site and into the V5 epitope. FIG. 16 shows the location of the TK polyA Reverse primer binding site.

Nucleic acid molecules of the invention amy be introduced into host cells (e.g., mammalian cells). For example, nucleic acid molecules in which a nucleic acid sequence encoding a polypeptide of interest is joined to a nucleic acid sequence encoding a polypeptide having a detectable activity such that a fusion polypeptide comprising all or a portion of both polypeptides can be expressed may be introduced into a host cell. Positive control vectors (e.g., pcDNA76.2/cGeneBLAZer m-GW/lacZ or pcDNA™ 6.2/nGeneBLAzer™-GW/lacZ) and a mock transfection (negative control) may be included in experiments to evaluate results.

Once expression clone have been generated, plasmid DNA may be isolated for transfection. Plasmid DNA for transfection into eukaryotic cells must be very clean and free from phenol and sodium chloride. Contaminants will kill the cells, and salt will interfere with lipid complexing, decreasing transfection efficiency. Suitable plasmid DNA can be prepared using the S.N.A.P.™ MiniPrep Kit (Invitrogen Corporation, Carlsbad, Calif. 10-15 μg DNA, Catalog no. K1900-01), the S.N.A.P.™ MidiPrep Kit (Invitrogen Corporation, Carlsbad, Calif. 10-200 μg DNA, Catalog no. K1910-01), or CsCl gradient centrifugation.

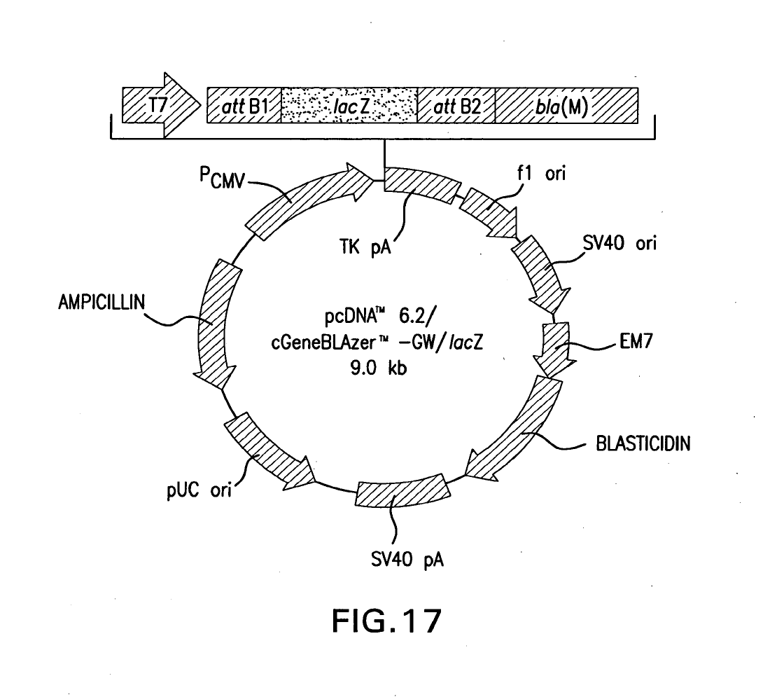

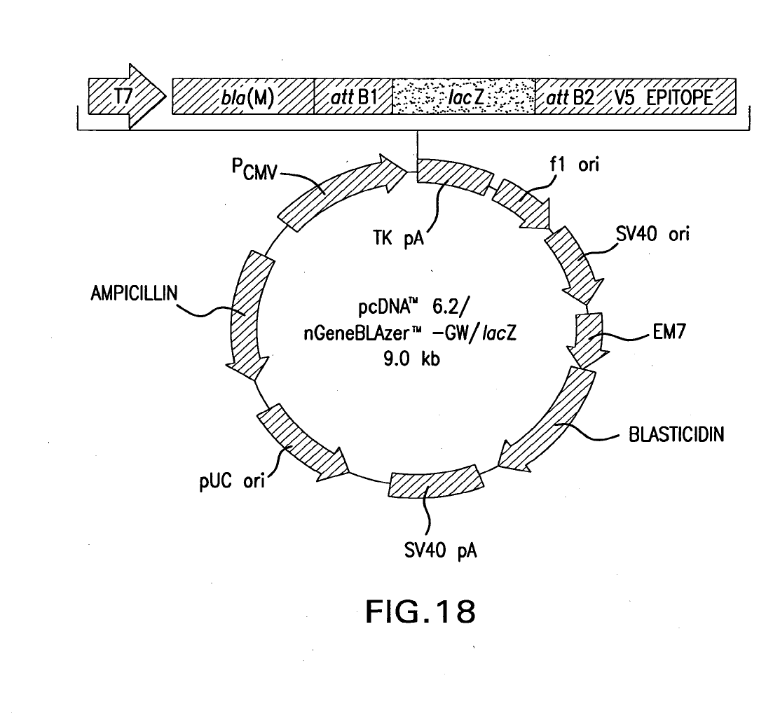

pcDNA™ 6.2/cGeneBLAzer™-GW/lacZ or pcDNA™ 6.2/nGeneBLAzer™-GW/lacZ can be used as positive control vectors for mammalian cell transfection and expression. FIGS. 17 and 18 provide maps. These vectors may be used to optimize recombinant protein expression levels in a particular cell line. These vectors allow expression of the β-galactosidase gene with either an N-terminal or C-terminal fusion to the β-lactamase reporter. These plasmids may be resuspended in 10 μl sterile water to prepare a 1 μg/μl stock solution. The stock solution can be used to transform a recA, endA E. coli strain like TOP10, DH5α, JM109, or equivalent. Transformants may be selected on LB agar plates containing 50-100 μg/ml ampicillin. A glycerol stock of a transformant containing plasmid may be prepared for long-term storage.

Cells may be transfected with the nucleic acid molecules of the invention using any technique known in the art, for example, those described in the preceding example.

Nucleic acid molecules of the invention (e.g., the pcDNA™6.2/GeneBLAzer™-DEST vectors) may contain the Blasticidin resistance gene to allow selection of stable cell lines. To create stable cell lines, transfect the construct into the cell line of choice (e.g., mammalian cell line of choice) and select for foci using Blasticidin.

Nucleic acid molecules of the invention (e.g., pcDNA™ 6.2/GeneBLAzer™-DEST constructs) may be linearized before transfection. While linearizing the vector may not improve the efficiency of transfection, it increases the chances that the vector does not integrate in a way that disrupts elements necessary for expression in host cells. To linearize the construct, cut at a unique site that is not located within a critical element or within the sequence encoding the polypeptide of interest.

To successfully generate a stable cell line expressing a polypeptide of interest, determine the minimum concentration of Blasticidin required to kill the untransfected host cell line by performing a kill curve experiment. Typically, concentrations ranging from 2.5 to 10 μg/ml Blasticidin are sufficient to kill most untransfected mammalian cell lines. Blasticidin is available separately from Invitrogen Corporation, Carlsbad, Calif. (Catalog no. R210-01). To perform a kill curve experiment, plate cells at approximately 25% confluence. Prepare a set of 6 plates. On the following day, replace the growth medium with fresh growth medium containing varying concentrations of Blasticidin (e.g. 0, 1, 3, 5, 7.5, and 10 μg/ml Blasticidin). Replenish the selective media every 3-4 days, and observe the percentage of surviving cells. Count the number of viable cells at regular intervals to determine the appropriate concentration of Blasticidin that prevents growth within 10-14 days after addition of Blasticidin.

Once the appropriate Blasticidin concentration to use for selection has been determined, stable cell lines can be prepared expressing polypeptides encoded by nucleic acid sequences present on nucleic acid molecules of the invention (e.g., pcDNA™ 6.2/GeneBLAzer™-DEST constructs).

Methods of preparing a stable cell may comprise transfecting a host cell (e.g., a mammalian cell line of interest) with one or more nucleic acid molecules of the invention (e.g., pcDNA™ 6.2/cGeneBLAzer™-DEST or pcDNA™6.2/nGeneBLAzer™-DEST expression constructs) using a transfection method of choice. Such methods may further include 24 hours after transfection, washing the cells and adding fresh growth medium; 48 hours after transfection, splitting the cells into fresh growth medium such that they are no more than 25% confluent; incubating the cells at 37° C. for 2-3 hours until they have attached to the culture dish; removing the growth medium and replacing with fresh growth medium containing Blasticidin at the predetermined concentration required for the cell line; feeding the cells with selective media every 3-4 days until Blasticidin-resistant colonies can be identified; and picking at least 5 Blasticidin-resistant colonies and expanding them to assay for recombinant protein expression. Cells should be plated at the indicated degree of confluence. If the cells are too dense, the antibiotic will not kill the cells. Antibiotics work best on actively dividing cells.

Methods of the invention may comprise detecting the presence or absence of a fusion protein by detecting one or more detectable activity. When the detectable activity is β-lactamase reporter activity, it may be detected in vivo or in vitro as described in the preceding example. Fusion polypeptides of the invention may also comprise a tag sequence that may be detected. For example, a polypeptide expressed from a pcDNA™ 6.2/nGeneBLAzer™-DEST expression construct that contains a sequence encoding a polypeptide of interest fused to the V5 epitope tag may be detected by Western blot analysis using Anti-V5 Antibodies. Suitable antibodies are commercially available, for example, from Invitrogen Corporation, Carlsbad, Calif. Any one of Anti-V5 Antibody (Catalog no. R960-25), Anti-V5-HRP Antibody (Catalog no. R961-25), or Anti-V5-AP Antibody (Catalog no. R962-25) can be used to detect the V5 epitope. In addition, the Positope™ Control Protein (Invitrogen Corporation, Carlsbad, Calif. Catalog no. R900-50) is available for use as a positive control for detection of fusion proteins containing a V5 epitope. The ready-to-use WestemBreeze® Chromogenic Kits and WesternBreeze® Chemiluminescent Kits are available from Invitrogen Corporation, Carlsbad, Calif. to facilitate detection of antibodies by colorimetric or chemiluminescent methods.

Expression of a protein fused to the β-lactamase reporter and/or to the V5 epitope tag will increase the size of the recombinant protein. Below are listed the increase in the molecular weight of a recombinant protein that can be expected from a particular fusion. Note that the expected sizes take into account any additional amino acids between the gene of interest and the fusion peptide.

| | | Expected Size |
|---|---|---|
| Vector | Fusion | Increase (kDa) |
| pcDNA ™ 6.2/cGeneBLAzer-DEST | β-lactamase | 30 kDa |
| | (C-terminal) | |
| pcDNA ™ 6.2/nGeneBLAzer-DEST | β-lactamase | 30 kDa |
| | (N-terminal) | |
| | V5 | 3 kDa |

If lacZ expressing vectors (e.g., pcDNA™ 6.2/cGeneBLAzer™-GW/lacZ or pcDNA™ 6.2/nGeneBLAzer™-GW/lacZ) are used as a positive control vectors, β-galactosidase expression can be assayed using techniques well known in the art. For example, β-galactosidase activity may be assayed by Western blot analysis or activity assay (see, Miller, J. H. (1972). Experiments in Molecular Genetics (Cold Spring Harbor, N.Y.: Cold Spring Harbor Laboratory). Commercially available antibodies and assays may be used. For example, Invitrogen Corporation, Carlsbad, Calif. offers A-Gal

Antiserum, the β-Gal Assay Kit, and the A-Gal Staining Kit for fast and easy detection of β-galactosidase expression.

### Example 4

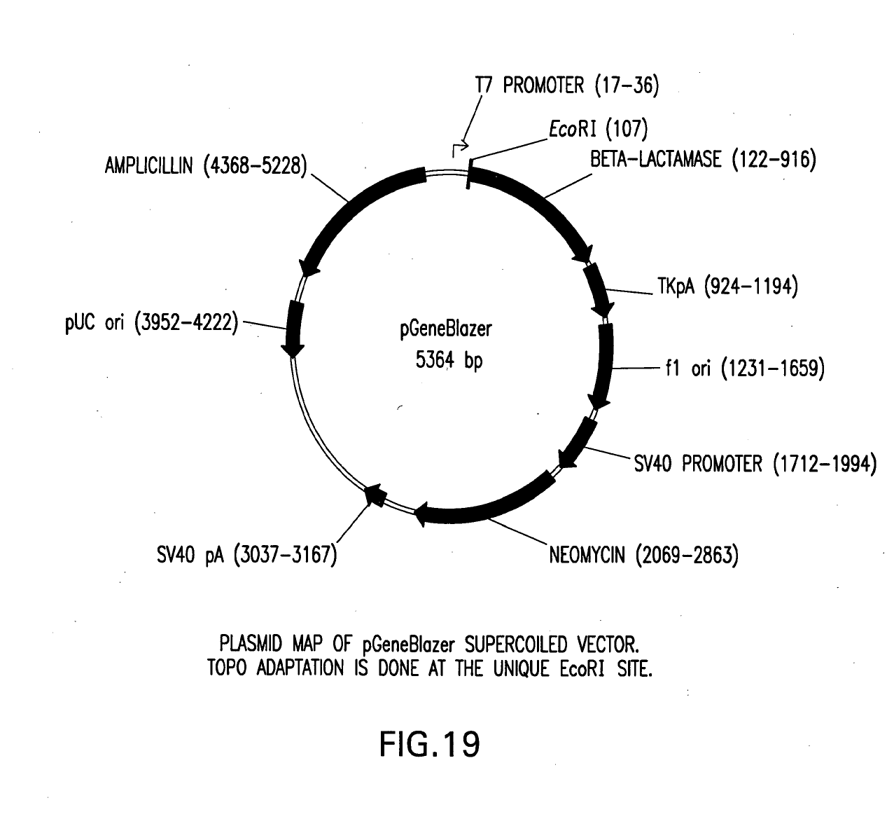

The β-lactamase gene, coupled with the CCF2 or CCF₄ substrate, is an excellent reporter system for promoter studies in mammalian cells. A “promoterless” β-lactamase vector (pGeneBlazer, FIG. 19) designed for promoter analysis in mammalian cells using β-lactamase activity as the readout has been created. It may be constructed as a bi-directional TOPO vector, allowing PCR amplification of one or more promoters of interest and cloning of the promoters upstream of the β-lactamase gene. Promoter activities can then be quantitatively measured both in vitro and in vivo, taking advantage of the ratiometric aspect of the CCF2 substrate (Whitney et al. (1998) Nat. Biotechnol. 16:1329-33; and Zlokarnik, et al. (1998) Science 279:84-88).

The β-lactamase reporter system is very versatile, allowing quantitative analyses in either live cells or in cell lysates (making it superior to luciferase or β-galactosidase assays), and the enzymatic nature of β-lactamase makes it more sensitive than GFP (less than 100 molecules of β-lactamase protein per cell are required for detection by eye). Live single cells can be analyzed with the cell-permeable CCF2-AM (or CCF4-AM, described below)) substrate either a) visually (expressing cells fluoresce blue, non-expressing cells fluoresce green), b) quantitatively (on a fluorescence microplate reader) or c) by FACS analysis (including the ability to quantitatively sort expressing cells from non-expressing cells). Alternatively, the CCF2-FA (free acid) form of the substrate can be used directly in traditional cell lysates and quantitated with a fluorescence microplate reader. In all cases, the fact that both the uncatalyzed substrate and the catalyzed product are fluorescent (green and blue, respectively) allows all data to be ratiometric and reported as a blue/green ratio. This automatically minimizes interference from variations in cell size, probe concentration, excitation intensity and emission sensitivity (Zlokarnik et. al. 1998). Maximum excitation of CCF substrates is 409 nm. Green emission is 520 nm and blue emission is 447 nm n.

There are currently two versions of the substrate available: CCF2 and CCF₄. Functionally both are similar, emitting green fluorescence prior to catalysis by β-lactamase and emitting blue fluorescence after. CCF₄ may be more stable in aqueous solution, making it more attractive to large volume high-throughput users. However, for ordinary use CCF2 and CCF₄ are indistinguishable.

CCF2 comes in two different forms: CCF2-AM and CCF2-FA. CCF2-AM is the ester form of the substrate, which is hydrophobic and capable of crossing live cell membranes—allowing it to be used in vivo. Once inside the cell, the endogenous cellular esterases remove the ester groups from CCF2-AM making it charged and hydrophilic. This causes the substrate to be trapped inside the cell and results in cells “loading” with more and more substrate over time, increasing the sensitivity of the assay without requiring higher concentrations of substrate. CCF2-FA is the free acid form of the substrate. This is essentially CCF2-AM with the ester groups removed, making it water soluble and appropriate for adding directly to cell lysates for enzymatic studies.

Examples are provided of TOPO-cloning three known mammalian promoters and quantitating their expression levels in whole live cells and in cell lysates.

Materials and Methods:

Cloning β-lactamase Gene

The β-lactamase gene was generated from the amp^{R} gene in pCMV/myc/nuc, with PCR forward primer 5′-CACCATGGACCCAGAAACGCTGGTGAAAG-3′ and PCR reverse primer 5′-CGATTACTTACCAATGCTTAATCAGTGAGG-3′. PCR amplified product was cloned into pCR-Blunt TOPO vector (Invitrogen Corporation, Carlsbad, Calif.) and sequence confirmed.

Generate pGeneBlazer Vector

The β-lactamase gene was released from pCR-Blunt with EcoRI and EcoRV digestion. Vector backbone was generated from pGlow template (Invitrogen Corporation, Carlsbad, Calif.) with EcoRI and XmaI digestion (to remove GFP and BGHpA). TKpA was generated from pcDNA3.2 (Invitrogen Corporation, Carlsbad, Calif.) with PmeI and XmaI. Expected size fragments from these digestion were purified from 1.2% E-Gel. The purified fragments were ligated and transformed into TOP 10 cells and plated on LB/Amp plates. The cloning junctions were sequence confirmed. The final vector is called pGeneBlazer (FIG. 19).

TOPO Charging

Bi-directional TOPO Charging at EcoRI was performed using published protocols (see Heyman, et al. Genome Research 9:383-392 (1999).

Comparison of the cloning efficiency of UbC promoter vs. 750 bp test insert

The UbC promoter was amplified from pUb6/V5HisB (Invitrogen Corporation, Carlsbad, Calif.) template with forward primer 5′-GACGGATCGGGAGATCTGG-3′ and reverse primer 5′-GGTACCAAGCTTCGTCTAAC-3′ (expected size: 1241 bp). The PCR conditions were the same for UbC and the test insert.

| | | Final |
|---|---|---|
| Components | Volume | Concentration |
| dH₂O | 36 μl | |
| 10 mM dNTP mixture (2.5 mM each) | 4 μl | 0.2 mM each |
| 10X PCR Buffer | 5 μl | 1X |
| 50 mM MgSO₄ | 1.5 μl | 1.5 mM |
| Primer 1 (100 ng/μl) | 1 μl | |
| Primer 2 (100 ng/μl) | 1 μl | |
| Template (10 ng/μl) | 1 μl | |
| Taq | 0.5 μl | |

For test insert, 100 ng template was used for PCR

PCR conditions were as follows: 94° C. 2 min (1 cycle); 94° C. 30 sec->55° C. 30 sec->72° C. 60 sec (25 cycles); 72° C. 2 min (1 cycle); and 4° C.

TOPO Cloning reactions contained the following components: PCR product 1 μl; Salt solution 1 μl; TOPO charged Vector: 1 μl, and dH₂O:3 μl.

Two μl of the cloning products were transformed into TOP 10, and 10 μl were plated on LB/Amp plate. As controls, we also transformed PCR products without cloning to check the background from original template.

Cloning CMV, CMV/TetO, and UbC promoters

CMV, CMV/TetO and UbC promoters were generated by PCR. PCR conditions for the promoters were as following:

| | | Final |
|---|---|---|
| Components | Volume | Concentration |
| dH₂O | 35.5 μl | |
| 10 mM dNTP mixture (2.5 mM each) | 4 μl | 0.2 mM each |
| 10X High Fidelity PCR Buffer | 5 μl | 1X |
| 50 mM MgSO₄ | 2 μl | 2 mM |
| Primer 1 (100 ng/μl) | 1 μl | |
| Primer 2 (100 ng/μl) | 1 μl | |
| Template (10 ng/μl) | 1 μl | |
| Platinum Taq High Fidelity (5 U/μl) | 0.5 μl | |

PCR reactions were performed as follows: 94° C. 4 min (1 cycle); 94° C. 30 sec->55° C. 30 sec->68° C. 90 sec (30 cycles); 68° C. 10 min (1 cycle); and 4° C. hold.

One microliter of unpurified PCR products was used for TOPO cloning and transformed into TOP 10, plated on LB/Amp plates.

Transfection and expression in mammalian cells pGeneBlazer/CMV, pGeneBlazer/CMVTetO, pGeneBlazer/UbC were transfected into GripTite 293 cells using the “rapid” 96-well transfection protocol (Lipofectamine2000 Product Manual, Invitrogen Corporation, Carlsbad, Calif.). Briefly, 320 ng of each DNA was diluted into 25 μl OPTI-MEM I in 96-well cell culture plates with black wall and clear bottom (Costar, cat No. 3603). For each well, 0.6 μl of Lipofectamine 2000 was diluted into 25 μl OPTI-MEM I medium and incubated at room temperature for 5 min, then added to the diluted DNA in each well, mixed gently, and incubated at room temperature for 20 min to allow DNA-lipid complexes to form. A GripTite 293 cell suspension was prepared (8.5×10⁵ cells/ml) and 100 μl of cell suspension was added (8.5×10⁴ cells/well) to each of the wells containing the DNA-LF2000 Reagent complexes and mixed gently. The plates were incubated at 37° C., 10% CO₂ incubator for 24 hr.

Detection of β-lactamase activity

Solution A (1 mM CCF4-AM in dry DMSO) was prepared according to the manufacturer's protocol (PanVera). One ml of 6×CCF4-AM loading solution was prepared by adding 6 μl of Solution A to 60 μl of Solution B (100 mg/ml Pluronic-F127 in DMSO containing 0.1% Acetic Acid) followed by vortexing. Then this combined solution was added to 934 Pl Solution C with vortexing, for a final volume of 1 ml. The cells were washed with HBSS, then 100 μl HBSS was added to each well. 20 μl of 6× loading solution was added to the 100 μl of cells in buffer. Cells were incubated at room temperature, protected from light, for 60 minutes (for CCF2-AM) or 90 minutes (for CCF4-AM). Cells were observed under Fluorescence Microscopy equipped with β-lactamase filter (e.g., Omega Filters #XF106-2 excitation: 400DF15, dichroic 420DCLP, emission: 435ALP, or Chroma Filters #41031 excitation: HQ405/20x, dichroic: 425DCXR, emission: HQ430LP) and photographed. Exact excitation of CCF substrates is 409 nm, green emission is 520 nm and blue emission is 447 nm.

After photography, cells were rinsed with PBS and lysed with 60 μl of Tropix lysis solution (0.1 M KCl, 0.2% Triton X-100), fluorescence was measured on a Gemini-XS Fluorescence Microtiter Plate Reader (Molecular Devices) at excitation: 405±10 nm, emission (blue): 460±20 nm, emission (green): 530±15 nm. Cells can also be lysed with 1% NP-40 or 1% IGEPAL CA-630 (Sigma # 1-3021) or 0.5% CHAPS or sonicated or freeze/thaw with equivalent results.

The following volumes may be used with the indicated tissue culture plates:

| | 96-well | 48-well | 24-well | 12-well | 6-well |
|---|---|---|---|---|---|
| 6X Loading Solution | 15 μl | 40 μl | 100 μl | 150 μl | 250 μl |
| HBSS | 75 μl | 200 μl | 500 μl | 750 μl | 1250 μl |

Other volumes may also be used as indicated elsewhere herein.

Activity of β-lactamase can also be measured directly in pre-made cell lysates. For this, CCF2-FA is recommended since it is already de-esterified and readily soluble in aqueous solution. CCF2-FA should be used at a final concentration of 10 μM in lysates. A more detailed protocol for lysate experiments with CCF2-FA can be obtained directly from PanVera.

Results and Discussion:

The β-lactamase gene, when used in mammalian cells, has the first 23 amino acids removed from the bacterial ampicillin gene. This deletes the periplasmic secretion signal without affecting the enzymatic activity. After cloning and sequencing, we identified a silent single point mutation (nucleotide 54 of the ORF, where “A” of the ATG start codon is nucleotide #1) in vectors of the invention that carry a β-lactamase gene derived from Invitrogen's amp^{R} gene. This single nucleotide polymorphism does not change the amino acid sequence.

TOPO Charging and promoter cloning

Bi-directional TOPO charging was performed at the EcoRI site. The standard 750 bp “test insert” was PCR amplified with Taq polymerase, as was the UbC promoter. The results of TOPO cloning these two inserts are shown in below. Approximately >95% vectors contained insert.

| | Test | | UbC | | Test insert | w/oUbC | w/ovector | |
|---|---|---|---|---|---|---|---|---|
| | insert | | promoter | | cloning | cloning | only | |
| colonies from | 87 | 99 | 232 | 238 | 0 | 0 | 0 | 2 |
| 10 μl | | | | | | | | |

| | | | | | |
|---|---|---|---|---|---|
| colonies per | 2,790 | 7,040 | 0 | 0 | 30 |
| transformation | | | | | |

Expression clones for each promoter (pGeneBlazer/CMV, pGeneBlazer/CMVTetO or pGeneBlazer/UbC) were successfully generated by Topo cloning PCR products of CMV, CMVTetO or UbC. Platinum Hi-Fi was the PCR enzyme used for these reactions. Taq amplification gives higher Topo cloning numbers but has no proof-reading.

Expression and Analysis in Mammalian Cells

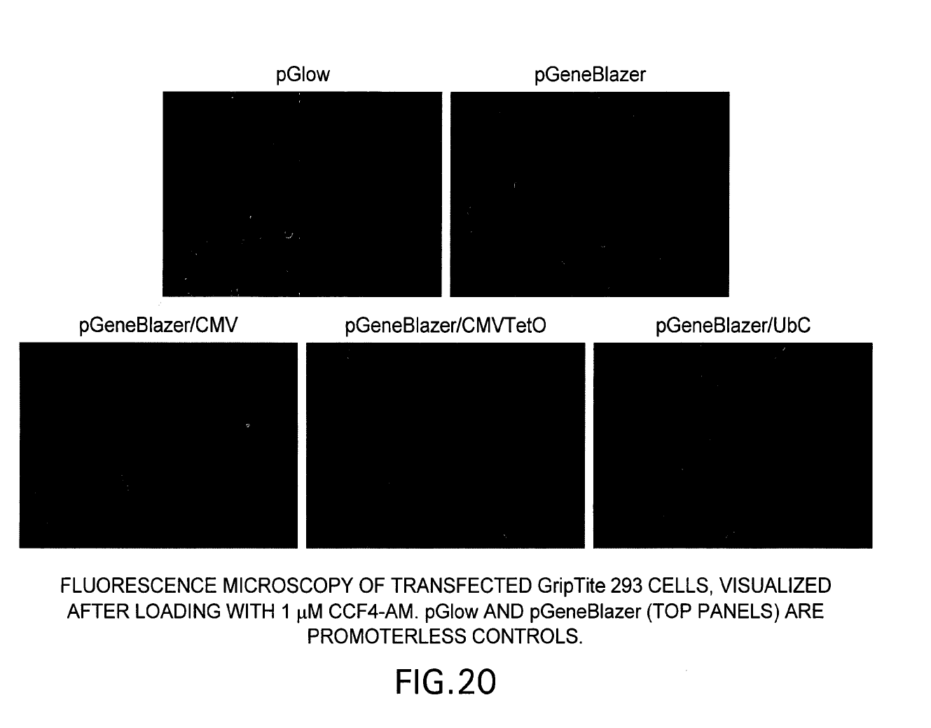

GripTite 293 cells were transiently transfected with each of the three promoter expression clones and β-lactamase activity was detected with the CCF4-AM substrate using both fluorescence microscopy and microplate reader quantitation (note that CCF2-AM and CCF4-AM perform equally in these experiments). As controls, the promoterless pGeneBlazer parent vector (supercoiled, no promoter cloned) and promoterless pGlow (Invitrogen Corporation, Carlsbad, Calif., supercoiled, no promoter cloned) were also transfected. Live transfected cells were loaded with CCF4-AM for ninety minutes and fluorescent photographs were taken under the microscope (FIG. 20). Very few blue cells were detected in the promoterless controls (FIG. 20, top panels) indicating the lack of β-lactamase activity. In cells transfected with pGeneBlazer containing a mammalian promoter, strong blue fluorescence was detected in >90% of the cells (FIG. 20, lower panels) indicating successful β-lactamase expression from each of the three promoters.

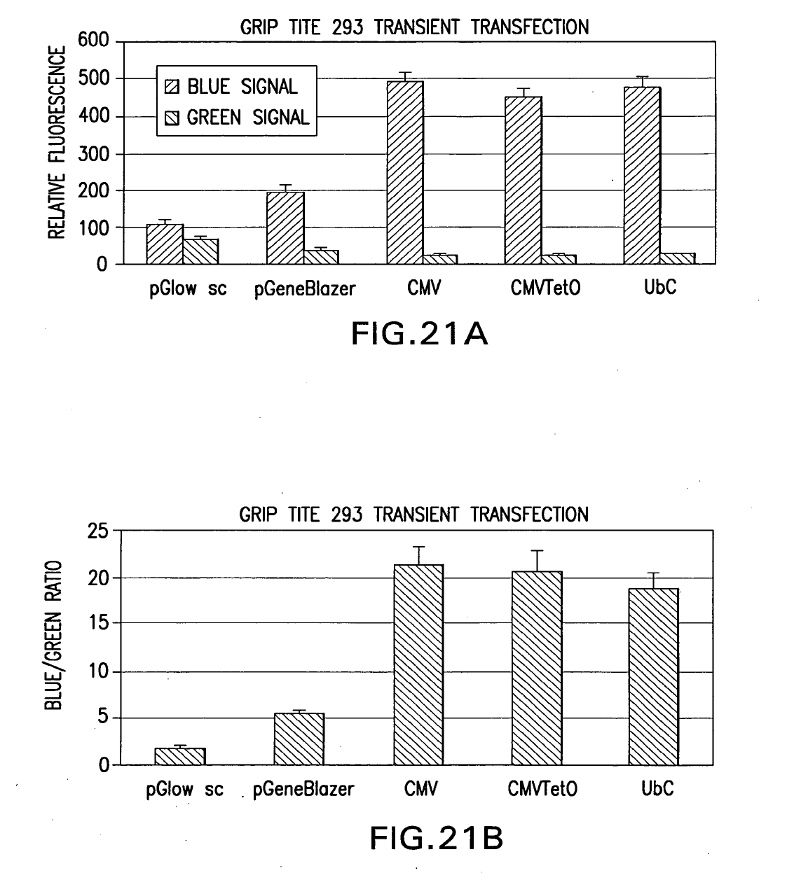

Quantification of promoter strength was performed by lysing the CCF₄-loaded transfected cells (from FIG. 20) and reading the lysates on a fluorescence microplate reader. Live cells were loaded with CCF4-AM, lysed with non-ionic detergent and then the homogenous lysates read on the plate reader. Ratiometric analysis was performed by first reading the plate in the green fluorescence channel (to measure uncatalyzed substrate), followed by reading the same wells again in the blue channel (to measure catalyzed product). The resulting data was first plotted with the green and blue channels separated (FIG. 21A) and then as a blue-to-green ratio (FIG. 21B). Expression strength of the three promoters was nearly identical, as would be expected in 293 cells.

Some α-lactamase activity was observed in cells transfected with the promoterless pGeneBlazer control (compared to the pGlow control, see FIGS. 20 and 21). This activity is not thought to be coming from the ampicillin resistance gene in the plasmid backbone because the pGlow control has that same configuration. There are several explanations for this phenomenon. 1) there could be extraneous transcriptional activity coming around the plasmid (perhaps from the SV40 promoter or elsewhere) that expresses the promoterless β-lactamase gene. Linearizing the vectors may provide a system with near-zero background activity in situations where needed; 2) plasmid copy number per cell in these experiments is very high, which certainly could contribute to higher backgrounds. One solution to this is to create stable cell lines where the number of copies will be much lower; 3) some of the transfected plasmid may have already stably integrated into the cell's genome. If the promoterless β-lactamase gene integrates downstream of an active promoter, expression amy be seen. Despite these possibilities, for most applications where a mammalian promoter is TOPO cloned upstream of the β-lactamase gene, expression is easily detected and quantitated over the promoterless control indicating that this vector will perform well in promoter analysis experiments.

### Example 5

The β-lactamase gene, coupled with the CCF2 substrate, is an excellent reporter and detection system for protein expression in mammalian cells (Whitney et al. (1998) Nat. Biotechnol. 16:1329-33; and Zlokarnik, et al. (1998) Science 279:84-88). Destination vectors have been developed for expressing either N- or C-terminal fusions of the β-lactamase ORF with your protein of interest in mammalian cells. These vectors are analogous to the popular mammalian N- and C-terminal GFP fusion vector products except that they are built in the pcDNA6.2 backbone (CMV expression, tk polyA, blasticidin resistance). These new vectors are called pcDNA6.2/nGeneBlazer-DEST and pcDNA6.2/cGeneBlazer-DEST, for N- and C-term β-lactamase fusions, respectively.

The β-lactamase reporter system is very versatile, allowing quantitative analyses in either live cells or in cell lysates (making it superior to luciferase or β-galactosidase assays), and the enzymatic nature of β-lactamase makes it more sensitive than GFP (less than 100 molecules of β-lactamase protein per cell are required for detection by eye; Zlokarnik et. al. 1998). Live single cells can be analyzed with the cell-permeable CCF2-AM substrate (or CCF4-AM, see below) either a) visually (expressing cells fluoresce blue, non-expressing cells fluoresce green), b) quantitatively (on a fluorescence microplate reader) or c) by FACS analysis (including the ability to quantitatively sort expressing cells from non-expressing cells). Alternatively, the CCF2-FA (free acid, see below) form of the substrate can be used directly in traditional cell lysates and quantitated with a fluorescence microplate reader. In all cases, the fact that both the uncatalyzed substrate and the catalyzed product are fluorescent (green and blue, respectively) allows all data to be ratiometric and reported as a blue/green ratio. This automatically minimizes interference from variations in cell size, probe concentration, excitation intensity and emission sensitivity (Zlokarnik et. al. 1998). Maximum excitation of CCF substrates is 409 nm. Green emission is 520 nm and blue emission is 447 nm.

There are currently two versions of the substrate available: CCF2 and CCF₄. Functionally both are similar, emitting green fluorescence prior to catalysis by β-lactamase and emitting blue fluorescence after. CCF₄ may be more stable in aqueous solution, making it more attractive to large volume high-throughput users. For most applications, however, CCF2 and CCF₄ are indistinguishable.

CCF2 comes in two different forms: CCF2-AM and CCF2-FA. CCF2-AM is the ester form of the substrate, which is hydrophobic and capable of crossing live cell membranes—allowing it to be used in vivo. Once inside the cell, the endogenous cellular esterases remove the ester groups from CCF2-AM making it charged and hydrophilic. This causes the substrate to be trapped inside the cell and results in cells “loading” with more and more substrate over time, increasing the sensitivity of the assay without requiring higher concentrations of substrate. CCF2-FA is the free acid form of the substrate. This is essentially CCF2-AM with the ester groups removed, making it water soluble and appropriate for adding directly to cell lysates for enzymatic studies.

Examples are provided of GATEWAY® cloning a test gene (lacZ) and showing expression levels similar to a standard pcDNA™-lacZ vector, and quantitating gene expression levels in whole live cells and in cell lysates using the CCF2 substrate.

Materials and Methods:

Destination Vector Cloning:

Construction of pcDNA6.2/V5-2/FLS-1 (intermediate vector).

The 4685 bp SnaBI/Age I fragment from pcDNA6.2-DEST (Invitrogen Corporation, Carlsbad, Calif.) was ligated with the 455 bp SnaBI/AgeI fragment from pcDNA3.1V5HisA (Invitrogen Corporation, Carlsbad, Calif.) to generate pcDNA6.2-MCS. A 143 bp HindIII/AgeI fragment was removed from pcDNA6.2-MCS and replaced with the synthetic V5-2/FLS-1 polylinker to create pcDNA6.2/V5-2/FLS-1. The sequences of the oligonucleotides forming the V5-2/FLS-1 polylinker were:

5′ AGCTGAGCGCTGTTAACGGGAAGCCTATCCCTAACCC TCTCCTCGGTCTCGATTCTACGCGTA 3′ (sense strand) (SEQ ID NO:121) and
5′CCGGTACGCGTAGAATCGAGACCGAGGAGAGGGTTAG GGATAGGCTTCCCGTTAACAGCGCTC 3′ (complementary strand) (SEQ ID NO:122). Clones of pcDNA6.2NV5-2/FLS-1 were verified by restriction endonuclease digestion patterns and DNA sequencing analysis.

Construction of pcDNA6.2/nGeneBlazer-DEST

The frame B GATEWAY® conversion cassette (Invitrogen Corporation, Carlsbad, Calif.) was inserted into HpaI site of pcDNA6.2/V5-2/FLS-1 to create pcDNA6.2-HpaI-Dest. The β-lactamase gene was amplified by PCR with the oligonucleotides ntermbla5 (5′CACCATGGACCCAGAAACGCTGGT 3′ (SEQ ID NO:123)) and ntermbla3 (5′ CAATGCTTAATCAGTGAGGC 3′ (SEQ ID NO:124)) using pUC19 as the template and cloned into the Eco4711 site of pcDNA6.2-HpaI-Dest to create pcDNA6.2/nGeneBlazer-Dest (FIG. 14). Clones of pcDNA6.2/nGeneBlazer-Dest were verified by restriction endonuclease digestion patterns and DNA sequencing analysis.

Construction of pcDNA6.2/cGeneBlazer-DEST

The frame B GATEWAY® conversion cassette was inserted into Eco471II site of pcDNA6.2/V5-2/FLS-1 to create pcDNA6.2-Eco47III-Dest. pcDNA6.2-Eco47III-Dest was linearized with HpaI and Agel and the DNA ends made blunt with T4 DNA polymerase in the presence of all four dNTPs in preparation for the insertion of a β-lactamase gene to create pcDNA6.2/cGeneBlazer-Dest (FIG. 13). The β-lactamase gene was PCR amplified with the oligonucleotides ctermbla5 (5′ ATGGACCCAGAAACGCTGGT 3′ (SEQ ID NO:125)) and ctbla3stop (5′ TTACCAATGCTTAATCAGTG 3′ (SEQ ID NO:126)) using pUC19 as the template. Clones of pcDNA6.2/cGeneBlazer-Dest were verified by restriction endonuclease digestion patterns and DNA sequencing analysis.

Creation of Expression Control Vectors

pcDNA6.2/nGeneBlazer-GW/lacZ was generated by standard GATEWAY® LR reaction between pENTR/SD-lacZ(stop) (Invitrogen Corporation, Carlsbad, Calif.) and pcDNA6.2/nGeneBlazer-DEST. pcDNA6.2/cGeneBlazer-GW/lacZ was generated by LR reaction of pENTR-lacZ(no stop) (Invitrogen Corporation, Carlsbad, Calif.) with pcDNA6.2/cGeneBlazer-DEST. Correct clones for each expression control were verified by restriction digest and cloning junctions were sequence verified.

Both DEST vectors were assayed to measure colony output and to detect ccdB mutants and plasmid contamination.

Expression and Analysis:

GripTite 293, CHO or COS-7 cells were plated in 24-well plates and transiently transfected using Lipofectamine 2000, following the manufacturer's recommended protocol (Invitrogen Corporation, Carlsbad, Calif.). 48 hours post transfection, cells were either 1) labeled with CCF4-AM to detect β-lactamase activity (see protocol below), 2) fixed and stained for β-galactosidase expression using the Beta-galactosidase Staining Kit (Invitrogen Corporation, Carlsbad, Calif.), 3) harvested for Tropix β-galactosidase activity assay (PE Biosystems), or 4) harvested for anti-lacZ western blotting (4-12% NuPage Bis-Tris gel and WESTERN BREEZE™ Kit, Invitrogen Corporation, Carlsbad, Calif.).

In vivo β-lactamase detection using CCF4-AM was performed as follows. Twenty-four hours post transfection, cells were trypsinized and re-plated into black-walled clear-bottom 96-well plates (Costar #3603) at 4.5×10⁴ cells/well in 100 μl complete media. The following day, cells were loaded by adding 20 μl 6×CCF4-AM loading solution into each well (wells already contain 100 μl complete media and cells, final volume was 120 μl) and incubating for 90 minutes at room temperature. One ml of 6×CCF4-AM loading solution was prepared by adding 12 μl of Solution A (1 mM CCF4-AM in dry DMSO) to 60 μl of Solution B (100 mg/ml Pluronic-F127 in DMSO containing 0.1% Acetic Acid) followed by vortexing. Then this combined solution was added to 934 μl Solution C with vortexing, for a final volume of 1 ml (final CCF4-AM concentration was 12 μM in the 6× stock, 2 μM final on cells). After 90 minutes loading at room temperature, cells were observed under fluorescence microscopy equipped with β-lactamase filters (e.g., Omega Filters #XF106-2 excitation: 400DF15, dichroic 420DCLP, emission: 435ALP, or Chroma Filters #41031 excitation: HQ405/20x, dichroic: 425DCXR, emission: HQ430LP) and photographed. Exact excitation of CCF substrates is 409 nm, green emission is 520 nm and blue emission is 447 nm.

Results and Discussion:

Destination Vector QC

Vectors pcDNA6.2/nGeneBlazer-DEST and pcDNA6.2/cGeneBlazer-DEST were assayed for colony output and ccdB mutations.

| | | | Values | | Values | |
|---|---|---|---|---|---|---|
| Sample | Criteria | | pcDNA6.2/nGeneBlazer | | pcDNA6.2/cGeneBlazer | |
| Cells only | 0 | cfu/ng DNA | 0 | cfu/ng DNA | 0 | cfu/ng DNA |
| No DNA | 0 | cfu/ng DNA | 0 | cfu/ng DNA | 0 | cfu/ng DNA |
| DEST vector | <1100 | cfu/ng DNA | 110 | cfu/ng/DNA | 220 | cfu/ng/DNA |
| only | | | | | | |
| L × R Reaction | ≧1.65 × 10⁶ | cfu/ng DNA | 3.19 × 10⁶ | cfu/ng DNA | 4.466 × 10⁶ | cfu/ng DNA |
| (n = 2) | | | | | | |
| pUC19 only | ≧7.5 × 10⁸ | cfu/ng DNA | 2.42 × 10⁸ | cfu/ng DNA | 5.61 × 10¹⁰ | cfu/ng DNA |
| (n = 2) | | | | | | |

The ccdb assay yielded the following results.

| | | | | |
|---|---|---|---|---|
| | | | Transformation | Transformation |
| | | | Efficiency | Efficiency |
| | Cell | | pcDNA6.2/nGene | pcDNA6.2/cGene |
| Sample | Type | Antibiotic | Blazer | Blazer |

| | | | | | | |
|---|---|---|---|---|---|---|
| Cells Only | DB3.1 | Amp | 0 | cfu/ug DNA | 0 | cfu/ug DNA |
| | | Kan | 0 | cfu/ug DNA | 0 | cfu/ug DNA |
| pUC19 only (n = 4) | DB3.1 | Amp | 1.25 × 10⁷ | cfu/ug DNA | 6.0 × 10⁶ | cfu/ug DNA |
| DEST vector only | DB3.1 | Amp | 2.5 × 10⁶ | cfu/ug DNA | 8.45 × 10⁷ | cfu/ug DNA |
| (n = 4) | | | | | | |
| Cells Only | TOP10 | Amp | 0 | cfu/ug DNA | 0 | cfu/ug DNA |
| | | Kan | 0 | cfu/ug DNA | 0 | cfu/ug DNA |
| pUC19 only (n = 4) | TOP10 | Amp | 1.5 × 10⁸ | cfu/ug DNA | 3.65 × 10⁸ | cfu/ug DNA |
| DEST vector only | TOP10 | Amp | 3.0 × 10³ | cfu/ug DNA | 2.0 × 10³ | cfu/ug DNA |
| (n = 4) | | Kan | 0 | cfu/ug DNA | 0 | cfu/ug DNA |

| | | |
|---|---|---|
| Fold-killing | 1 × 10⁴ | 2.57 × 10⁶ |
| (criteria = 1 × 10⁴) | Pass | Pass |

Expression and Analysis in Mammalian Cells

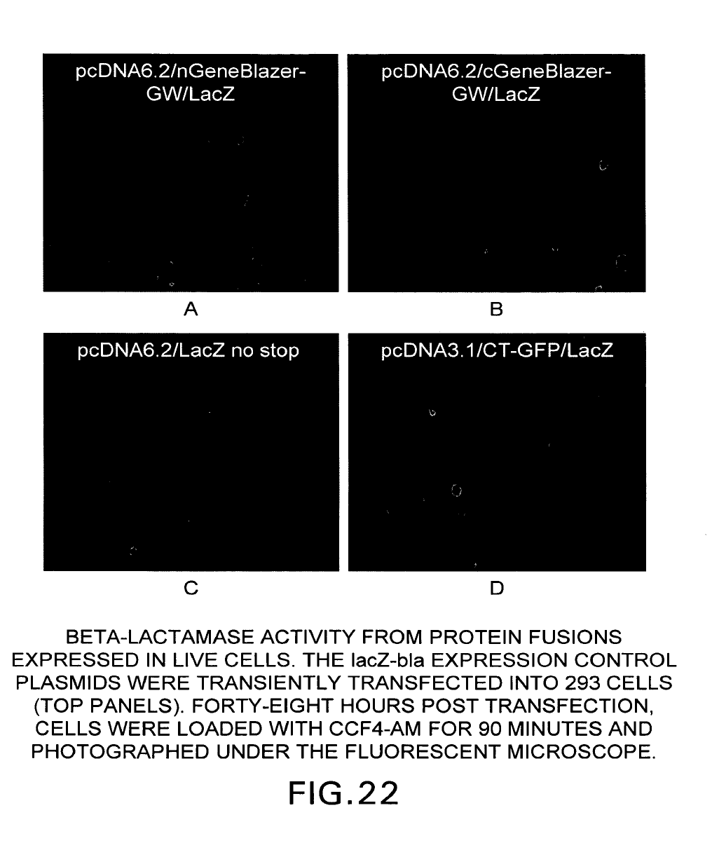

GripTite 293 cells (Invitrogen Corporation, Carlsbad, Calif.) were transiently transfected with each of the fusion controls (pcDNA6.2/nGeneBlazer-GW/lacZ and pcDNA6.2/cGeneBlazer-GW/lacZ), which express the β-lactamase ORF fused to either the N- or C-terminus of the lacZ ORF. Forty-eight hours post transfection, cells were loaded with CCF4-AM (see Materials and Methods) and photographed under fluorescence microscopy (FIG. 22). β-lactamase activity from the expressed fusion proteins was readily detectable as blue fluorescent cells (FIG. 22, upper panels). Transfected expression controls (pcDNA6.2/lacZ and pcDNA3.1/CT-GFP/lacZ) did not show any β-lactamase activity, as expected, demonstrated by the lack of blue cells (FIG. 22, lower panels). All transfected plasmids showed similar β-galactosidase staining indicating comparable transfection efficiencies in all samples (˜50% transfection efficiency in all wells). It is noteworthy that the two expression controls (indeed all plasmids in this experiment) contain the ampicillin resistance gene in their plasmid backbones, indicating that no detectable β-lactamase activity comes from the bacterial amp^{R} expression cassette. This experiment was repeated in CHO cells with identical results.

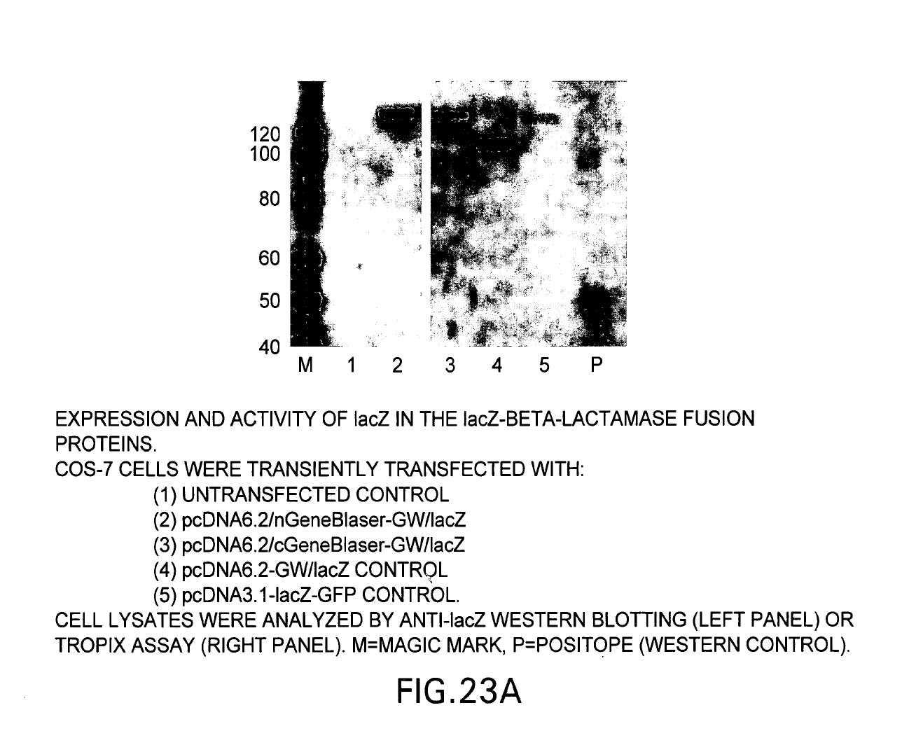

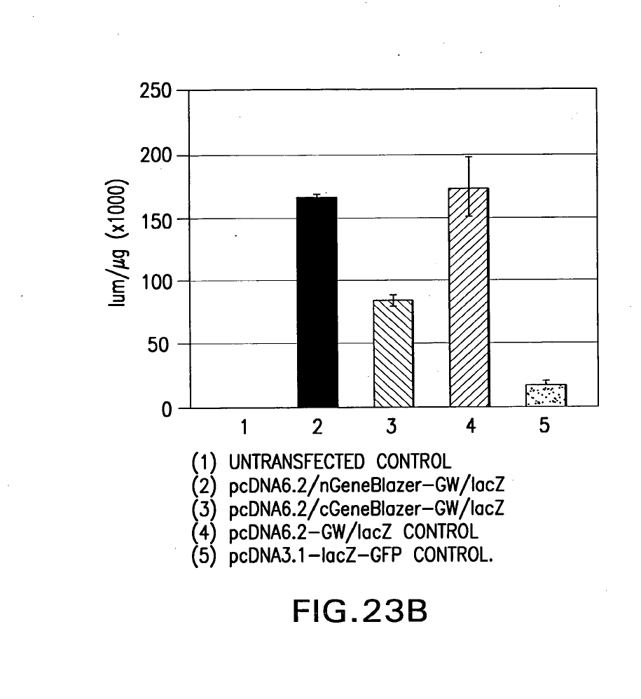

COS-7 cells were also transiently transfected with each of the fusion expression controls. Forty-eight hours post transfection, cells were lysed and analyzed by either anti-lacZ western blotting (FIG. 23, left panel) or β-galactosidase activity (right panel). Fusion proteins between β-lactamase and lacZ were detected on the western blot migrating at the correct molecular weight (nβ-lac/lacZ, lane 2 and lacZ/cβ-lac, lane3). Control transfections of pcDNA6.2-GW/lacZ (native lacZ, lane 4) and pcDNA3.1-lacZ/GFP (lacZ/GFP fusion, lane 5) also properly expressed proteins of the expected molecular weights. β-galactosidase activity was quantitated from similar lysates (FIG. 23, right panel). Activity measured from the N-terminal β-lac/lacZ fusion was comparable to native lacZ (compare lanes 2 and 4). Activity from the C-terminal fusion (lane 3) was approximately 50% of the N-term and the native lacZ. This drop in activity correlates with slightly reduced signal on the western blot (right panel, lane 3). This is a common phenomenon observed with lacZ, where fusing additional protein sequences on its C-terminal often reduces expression and activity levels. This has been observed with V5His and mycHis C-terminal tags and was also seen here with a lacZ/GFP fusion (FIG. 23, lane 5). This experiment has been repeated (along with an internal normalizing luciferase transfection control) with identical results.

Compatibility with Tag-On-Demand

The C-terminal GeneBlazer vector (pcDNA6.2/cGeneBlazer-DEST) is compatible with Tag-On-Demand provided that the GATEWAY®-cloned ORF has a TAG stop codon. For example, if an ORF is chosen from the Ultimate ORF collection (Invitrogen Corporation, Carlsbad, Calif.) and GATEWAY® cloned into this DEST vector, expression of the ORF-β-lactamase fusion protein will be dependent on tRNA suppression from Tag-On-Demand. Of course if the ORF does not contain a stop codon, the fusion protein will be expressed 100% of the time.

The N-terminal GeneBlazer vector will always express a fusion protein (β-lac/ORF). Fusion to the C-terminal V5 antibody epitope tag requires an ORF with no stop codon. If the ORF contains a TAG stop codon, Tag-On-Demand can be used to express a β-lac/ORF/V5 fusion protein. This may be useful if no convenient antibodies are available for the ORF.

### Example 6

Mammalian GeneBlazer vectors encode the β-lactamase gene for expression and other analyses. The construction of pENTR/GeneBlazer™, a GATEWAY® Entry vector designed to be a source of β-lactamase for transfer of the reporter into any Destination (R1R2) vector in an LR reaction is described below.

Materials and Methods

Cloning of pENTR/GeneBlazer

The β-lactamase gene was amplified using the PCR primers b1β and b2pTAGA and pUC19 as the template. This PCR amplified fragment was reacted with pDONR221 (Invitrogen Corporation, Carlsbad, Calif.) in a BP CLONASE™ reaction to create pENTR/GeneBlazer™, the Entry clone contains the T-lactamase ORF with a CACC Kozak consensus sequence and a TAG stop codon. The final Entry clone was verified by endonuclease digestion profile and DNA sequence analysis. A pENTRJGeneBlazerNS (no stop variant) was also created.

Primer sequences:

(SEQ ID NO:127)

| | |
|---|---|
| b1β: | 5′ GGG GAG AAG TTT GTA GAA AAA AGG AGG GAG |
| | GAG CAT GGA CCC AGA AAC GCT GGT GA 3′ |

(SEQ ID NO:128)

| | |
|---|---|
| b2βTAGA: | 5′ GGG GAG GAG TTT GTA GAA GAA AGG TGT CTA |
| | GCA ATG CTT AAT GAG TGA GGG A 3′ |

Creation of Expression Control Vectors

pcDNA6.2/FRTNV5-2-GW/GeneBlazer was generated by standard GATEWAY® LR reaction between pENTR/GeneBlazer™ and pcDNA6.2/FRT/V5-2-DEST.

Expression and Analysis:

GripTite 293 cells were plated in 24-well plates and transiently transfected using Lipofectamine 2000, following the manufacturer's recommended protocol (Invitrogen Corporation, Carlsbad, Calif.). Twenty-four hours post transfection, cells were trypsinized and re-plated into black-walled clear-bottom 96-well plates (Costar #3603) at 3×10⁵ cells/well in 100 μl complete media. The following day, cells were loaded by adding 20 μl 6×CCF4-AM loading solution into each well (wells already contain 100 μl complete media and cells, final volume was 120 μl) and incubating for 90 minutes at room temperature. One milliliter of 6×CCF4-AM loading solution was prepared by adding 12 μl of Solution A (1 mM CCF4-AM in dry DMSO) to 60 μl of Solution B (100 mg/ml Pluronic-F127 in DMSO containing 0.1% Acetic Acid) followed by vortexing. Then this combined solution was added to 940 μl Solution C with vortexing, for a final volume of 1 ml (final CCF4-AM concentration was 12 μM in the 6× stock, 2 μM final on cells). After 90 minutes loading at room temperature, cells were observed under fluorescence microscopy equipped with β-lactamase filters (e.g., Omega Filters #XF106-2 excitation: 400DF15, dichroic 420DCLP, emission: 435ALP or Chroma Filters #41031 excitation: HQ405/20x, dichroic: 425DCXR, emission: HQ430LP) and photographed. Exact excitation of CCF substrates is 409 nm, green emission is 520 nm and blue emission is 447 nm.

Cells were photographed under fluorescence microscopy in the presence of the loading dye. After the photographs were taken, the loading solution was aspirated from the wells and the cells were washed one time with PBS. After the wash, 70 μl 1×PBS was added to all wells and the plate was read on a Molecular Devices Spectra Max Gemini XS plate reader at excitation: 405 nm, emission (blue): 460 nm, emission (green): 530 nm. Subsequent to the whole cell read on the plate reader, the PBS was aspirated and replaced with 70 μL Tropix lysis buffer (0.1 M KCl, 0.2% Triton X-100). The plate was allowed to sit for 5 minutes at room temperature, at which time the plate was again read on the plate reader at excitation: 405 nm, emission (blue): 460 nm, emission (green): 530 mm.

Results and Discussion

To create the expression vector pcDNA6.2/FRT/V5-2/GW-GeneBlazer, an LR reaction was performed with pENTR/GeneBlazer™×pcDNA6.2/FRT/V⁵-2/DEST. The reaction was transformed into TOP10 cells and plated on LB/Amp plates. 2.5×10⁵ colonies were obtained/per reaction. 10 colonies were screened by restriction analyses and 100% were found to be correctly recombined confirming that the att sites in pENTR/GeneBlazer™ are fully functional.

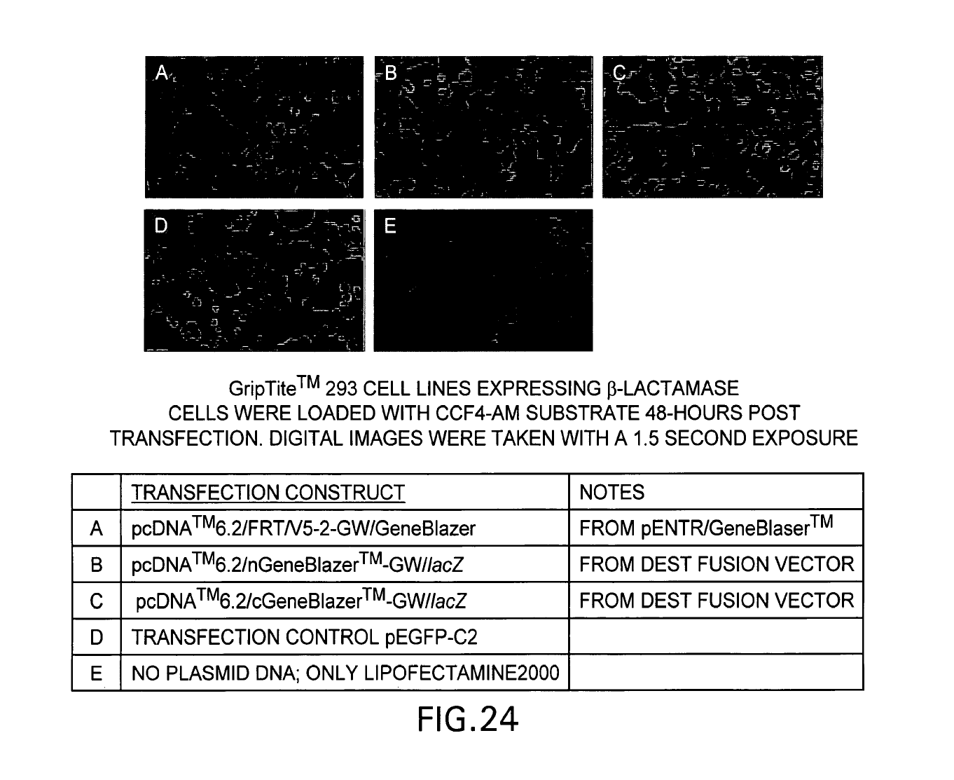

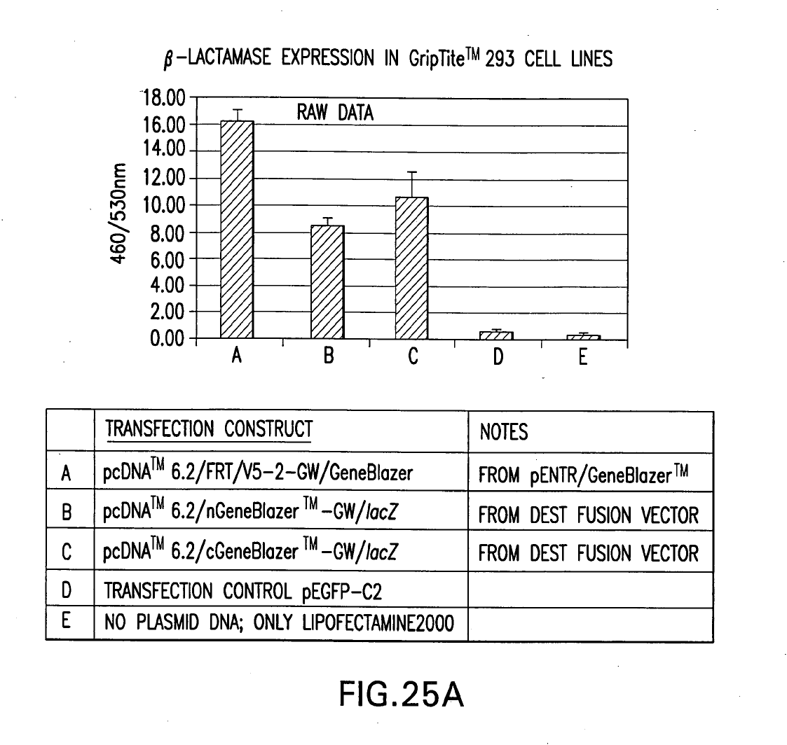

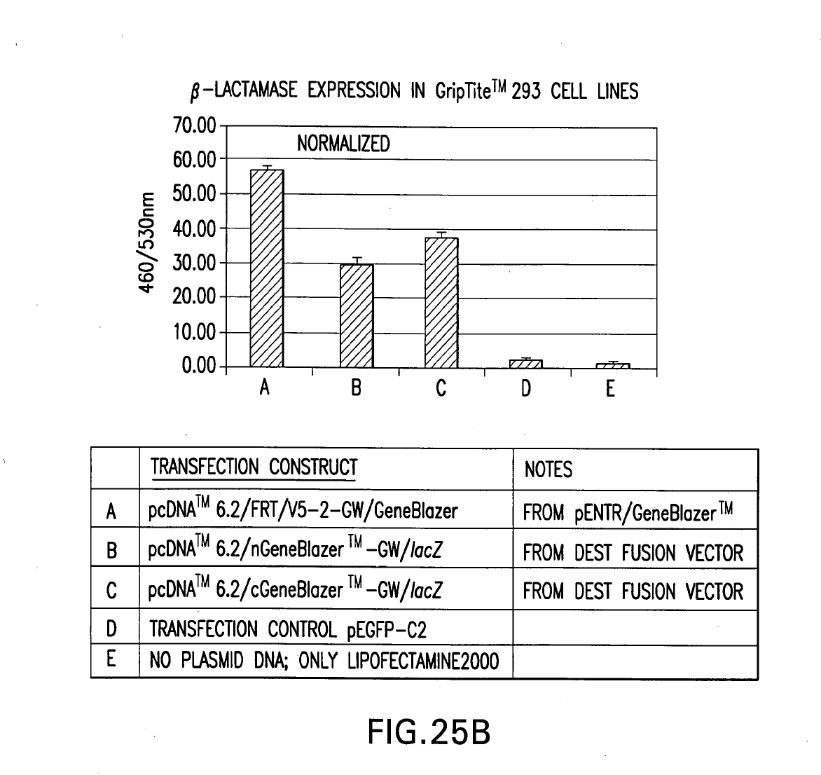

For expression analyses, GripTite 293 cells were transiently transfected with pcDNA6.2/FRT/V5-2-GW/GeneBlazer, alongside previously tested pcDNA6.2/nGeneBlazer-GW/lacZ and pcDNA6.2/cGeneBlazer-GW/lacZ. Forty-eight hours post transfection, cells were loaded with CCF4-AM and photographed under fluorescence microscopy (FIG. 24). β-lactamase expression from pcDNA6.2/FRT/V5-2-GW/GeneBlazer is comparable to β-lactamase expression from pcDNA6.2/nGeneBlazer-GW/lacZ and pcDNA6.2/cGeneBlazer-GW/lacZ (FIGS. 24 and 25). Expression controls pEGFP-C2 and mock, did not show any β-lactamase activity indicating that the β-lactamase activity comes from the BlaM gene and not the Amp^{R} gene in the vector backbone. Untagged β-lactamase (FIG. 25 lane A) expresses slightly better than tagged bla (FIG. 25, lanes B&C) as is often observed with fusion proteins.

### CONCLUSION

Sequence analyses confirmed the fidelity of pENTR/GeneBlazer™. The Entry clone is compatible with GATEWAY® as shown by colony-count and 100% LR cloning efficiency. In addition, pENTR/GeneBlazer™ has been tested by expression of β-lactamase in transfected mammalian cells by microscopy and quantitation of in vivo hydrolyzed enzyme substrate.

### Example 7

### Introduction

The β-lactamase gene, coupled with the CCF2 substrate, is an excellent reporter and detection system for protein expression in mammalian cells (Whitney et al. (1998) Nat. Biotechnol. 16:1329-33; and Zlokarnik, et al. (1998) Science 279:84-88).

Destination vectors have been developed for expressing either N- or C-terminal fusions of the β-lactamase ORF with a gene of interest in mammalian cells. These vectors are built in the pcDNA6.2 backbone (CMV expression, tk polyA, blasticidin resistance) and are called pcDNA6.2/nGeneBlazer-DEST and pcDNA6.2/cGeneBlazer-DEST, for N- and C-term β-lactamase fusions, respectively. To extend the cloning options, these vectors have been converted to topoisomerase charged vectors which will allow for quick directional cloning of PCR products.

Examples are provided of the construction of vectors in which: 1) the foreground to background colony count ratio from a Topo cloning reaction is 10 to 1 or better, 2) the Topo cloning efficiency is greater than 90% for presence and directionality of insert, 3) the cloned insert performs predictably in a BP CLONASE™ reaction, 4) the CAT gene has been Topo-cloned as a fusion to β-lactamase retain β-lactamase function and expression level.

Materials and Methods

Construction of pENTR Spec-ccdB D-Topo

The Spectinomycin and ccdB genes were amplified with the primers SC1 and SC2 using the vector pDEST6-R4R3-aadA (Invitrogen Corporation, Carlsbad, Calif.) as template. SC15′ CACCGACATTTTTGTTTAAACTT TGGTACCTGGATCCTTT-3′ (SEQ ID NO:129), SC2 5′ GACATTTTTGTTTAAACT TTGGTACCTGGATCCTTTAATTATTTGCCGACTACCTTGGT 3′ (SEQ ID NO:130). The PCR amplified fragment was Topo cloned into pENTR D-TOPO to generate pENTR Spec/ccdB. Clones were verified by restriction endonuclease digestion and DNA sequencing analysis.

Construction of pcDNA6.2/nGeneBlazer-GW/D.3 and pcDNA6.2/cGeneBlazer-GW/D.3

pENTR Spec/ccdB linearized with HpaI was reacted with either pcDNA6.2/nGeneBlazer-DEST linearized with EcoRI or pcDNA6.2/cGeneBlazer-DEST linearized with EcoRI in an LR reaction. A 211 aliquot of the LR reaction was transformed into DB3.1 cells and plated onto LB-Amp-Spec plates (Amp 100 μg/ml, Spec 100 μg/ml, Spectinomycin Sigma catalog number S-4014). The resulting clones, pcDNA6.2/nGeneBlazer-GW/D.3 and pcDNA6.2/cGeneBlazer-GW/D.3, were verified by restriction endonuclease digestion and DNA sequencing analysis.

BaeI Topo Adaptation Protocol

Twenty micrograms of either pcDNA6.2/nGeneBlazer-GW/D.3 or pcDNA6.2/cGeneBlazer-GW/D.3 were digested with 100 Units of Bael (NEB, lot #2) in a final volume of 250 μl. Any other restriciton enzyme known in the art may be also be used, for example, a Type II restriction enzyme such as a Type IIs restriction enzyme. The reaction was carried out in 1×NEBuffer 2 with 100 μg/ml of BSA and 20 μM S-adenosylmethionine at 37° C. for 6 hours. The Bael digest was terminated with the addition of 250 μl of Phenol/Chloroform (Invitrogen, Cat. #15593-031) and mixed vigorously. The organic and aqueous phases were separated by centrifugation at 14,000×g at 4° C. for 5 minutes. The aqueous (top) layer was transferred to a new tube and 25 μl of 3M sodium acetate (pH 5.2) was added and mixed. This was followed by 625 μl of 100% ethanol and incubated in ice for 5 minutes. Precipitated DNA was harvested by centrifugation at 14,000×g for 5 minutes at 4° C. The DNA pellet was washed with 500 μl of 70% ethanol, harvested by centrifugation at 14,000×g for 5 minutes at 22° C. The pellet was allowed to dry and then resuspended in 100 μl of TE. The DNA concentration was determined by its optical density at 260 nm.

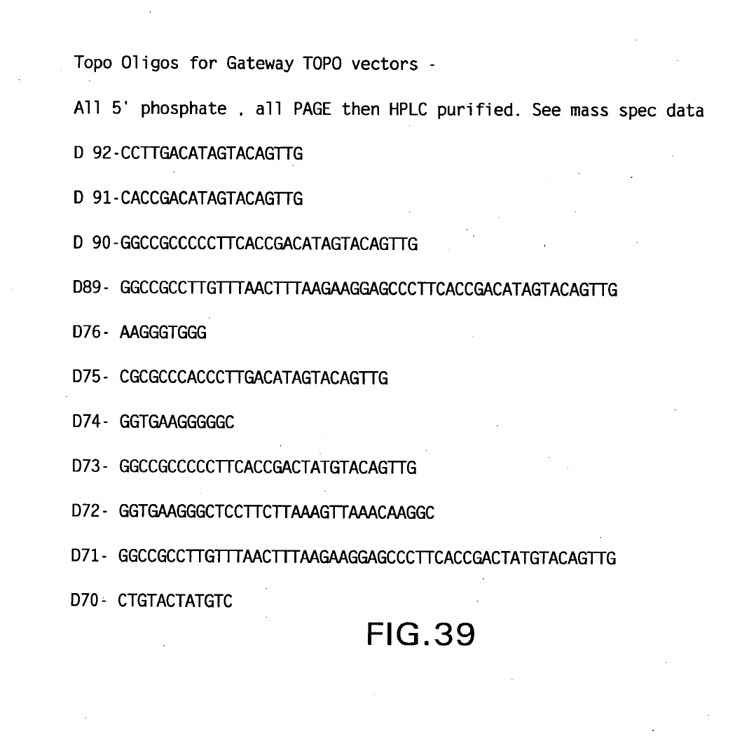

The sequences of the oligos used for Topo-charging are provided in FIG. 39.

For the Topo-charging reaction, 5 μg of BaeI linearized DNA was mixed with 1.5 μg of Topo-D70 Annealing oligo and 5 μg of Vaccinia DNA Topoisomerase in 1×NEBuffer #1 at a final volume of 50 μl. The reaction was incubated at 37° C. for 15 minutes. Then terminated with the addition of 5 μl of 10× Stop Buffer. The Topo charged vector was purified by gel electrophoresis (see, Heyman, et al. Genome Research 9:383-392 (1999)).

NotI/AscI adaptation protocol

Eighty micrograms of either pcDNA6.2/nGeneBlazer-GW/D.3 or pcDNA6.2/cGeneBlazer-GW/D.3 was digested with 480 units of NotI (NEB, lot # 49) in 400 μl of 1×NEBuffer #3 with 100 μg/ml of BSA (NEB) at 37° C. for 3 hours. This was followed by a phenol/chloroform extraction, DNA precipitation with sodium acetate and ethanol, and resuspension in 100 μl of water. The AscI digest was then performed with 480 units of AscI (NEB, lot#10) in 480 μl of 1×NEBuffer #4 at 37° C. for 3 hours. This was followed by a phenol/chloroform extraction, DNA precipitation with sodium acetate and ethanol, and resuspension in 50 μl of water. To this resuspended DNA the following. oligonucleotides were added, Topo D-90 (30 μg), Topo D-74 (14 μg), Topo D-75 (30 μg) and Topo D-76 (9 μg).

Ligation of the oligonucleotides to the NotI/AscI digested vector was performed in 150 μl of 1× Invitrogen T4 DNA ligase buffer with 20 U of Invitrogen T4 DNA ligase. The ligation reaction was performed at 14° C. for 16 hours. This was followed by a phenol/chloroform extraction, DNA precipitation with sodium acetate and ethanol, and resuspension in 175 μl of TE as described above. Excess oligonucleotides were removed with 3 sodium acetate/isopropanol precipitations and the final DNA pellet was resuspended in 42 μl of TE. The concentration of the final DNA solution was determined by agarose gel electrophoresis, ethidium bromide staining and estimation with a predetermined DNA mass ladder.

For the Topo-charging reaction, 5 μg of adapted DNA was mixed with 1.5 μg of Topo-D70 annealing oligo and 5 μg of Vaccinia DNA Topoisomerase in 1×NEBuffer #1 at a final volume of 50 μl. The reaction was incubated at 37° C. for 15 minutes. The reaction was terminated with the addition of 5 μl of 10× Stop Buffer. The Topo charged vector was purified by the Topo-vector Gel Purification protocol.

Topo Adaptation Efficiency Assay

The standard 750 bp D-Topo PCR product was used to assess the cloning efficiency of the Topo-charged β-lactamase fusion vectors. Twenty nanograms of PCR product was reacted with 1 μl of the Topo-charged vector in a final reaction volume of 6 μl. Two microliters of the reaction was used to transform 50 μl TOP10 cells and the number of colonies resulting from this transformation reaction counted. As a control reaction a similar reaction was performed without the PCR product added.

### TOPO Cloning of the CAT Gene

For TOPO cloning into Topoisomerase-charged pcDNA6.2/nGeneBlazer-GW/D.3, the CAT gene was amplified with the primers CATcacc and CATantiNS using pDEST6 as the PCR template. For TOPO cloning into Topoisomerase charged pcDNA6.2/cGeneBlazer-GW/D.3 the CAT gene was amplified with the primers CATcacc and CATantiS using pDEST6 as the PCR template. CATcacc 5′ CACCATGGAGAAAAAAATC ACTGG 3′, CATantiNS 5′CTACGCCCCGCCCTGCCACTCAT 3′, CATantiS 5′ CGCCCCGCCCTGCCACTCAT 3′. Standard Topo cloning reactions were performed with the PCR amplified CAT ORFs.

BP reactions with pcDNA6.2/nGeneBlazer-TopoCAT and pcDNA6.2/cGeneBlazer-TopoCAT

Both CAT expression vectors were digested with BglII prior to their use in a BP reaction. After a 3 hour incubation with BglII the digestion reaction was incubated at 80° C. for 90 minutes to inactivate the BglII enzyme. The BP reaction was performed with 150 ng of pDONR221, 50 ng of either pcDNA6.2/nGeneBlazer-TopoCAT or pcDNA6.2/cGeneBlazer-TopoCAT, 4 μl of BP CLONASE™, 4 μl of 10×BP reaction buffer in a final volume of 20 μl. The reaction was incubated at room temperature (22-25° C.) for 1 hour before the addition of 2 μl of Proteinase K (2 μg/μl) and incubated at 37° C. for 10 minutes to terminate the reaction. Two microliters of the reaction were used to transform 50 μl of TOP10 cells. 50 μl of the 500 μl grow out was plated onto LB-Kanamycin plates, incubated at 37° C. for 16 hours and the resulting colonies counted.

Expression analysis of pcDNA6.2/nGeneBlazer-TopoCAT and pcDNA6.2/cGeneBlazer-TopoCAT by in vivo β-lactamase activity detection

A total of four miniprep cultures of independently isolated clones were used to inoculate 100 mL midipreps denoted pcDNA6.2/nGeneBlazed/dTOPO/CAT # 32, pcDNA6.2/nGeneBlazer/dTOPO/CAT #36, pcDNA6.2/cGeneBlazer/dTOPO/CAT #38, and pcDNA6.2/cGeneBlazer/dTOPO/CAT #48. DNAs were isolated using the S.N.A.P. Midiprep kit (Invitrogen Corporation, Carlsbad, Calif.).

GripTite 293 cells were plated in 24-well plates and transiently transfected using Lipofectamine 2000, following the manufacturer's recommended protocol (Invitrogen Corporation, Carlsbad, Calif.). Forty-eight hours post transfection, cells were labeled with CCF4-AM to detect β-lactamase activity.

In vivo β-lactamase detection using CCF4-AM was performed as follows. Twenty-four hours post transfection, cells were trypsinized and re-plated into black-walled clear-bottom 96-well plates (Costar #3603) at 3×10⁵ cells/well in 100 μl complete media. The following day, cells were treated by addition of 20 μl 6×CCF4-AM loading solution to each well (wells already contain 100 μl complete media and cells, final volume was 120 μl) and incubation for 90 minutes at room temperature. One milliliter of 6×CCF4-AM loading solution was prepared by adding 12 μl of Solution A (1 mM CCF4-AM in dry DMSO) to 60 μl of Solution B (100 mg/ml Pluronic-F127 in DMSO containing 0.1% Acetic Acid) followed by vortexing. This combined solution was added to 940 μl Solution C with vortexing, for a final volume of 1 ml (final CCF4-AM concentration was 12 μM in the 6× stock, 2 μM final on cells). After 90 minutes loading at room temperature, cells were observed under fluorescence microscopy equipped with β-lactamase filter (excitation: HQ405/20x, dichroic mirror: 425 DCXR, emission: HQ435LP; Omega Filters #XF106-2) and photographed. Exact excitation of CCF substrates is 409 nm, green emission is 520 nm and blue emission is 447 nm.

Cells were photographed under fluorescence microscopy in the presence of the loading dye. B-lactamase activity from the expressed fusion proteins was readily detectable as blue fluorescent cells. After the photographs were taken, the loading solution was aspirated from the wells and the cells washed once with PBS. After the wash, 100 μl 1×PBS was added to all wells and the plate was read on a Molecular Devices Spectra Max Gemini XS plate reader (405±10 nm, emission (blue): 460±20 nm, emission (green): 530±15 nm). Subsequent to the whole cell read on the plate reader, the PBS was aspirated and replaced with 70 μl Tropix lysis buffer. The plate was allowed to sit for 5 minutes at room temperature, at which time the plate was again read on the plate reader.

Expression analysis of pcDNA6.2/nGeneBlazer-TopoCAT and pcDNA6.2/cGeneBlazer-TopoCAT by immuno-detection of β-lactamase-CAT fusions

COS-7 cells were seeded in 24-well format at a density of 8×10⁴ cells/well. COS-7 cells were plated with 500 μl of DMEM containing 10% FBS, 4 mM L-glutamine, and 0.1 mM non-essential amino acids. The following day the media was aspirated and fresh media was added prior to transfection.

The following are the vectors that were transfected in duplicate:

pcDNA6.2/nGeneBlazed/dTOPO/CAT # 32
pcDNA6.2/nGeneBlazer/dTOPO/CAT #36
pcDNA6.2/cGeneBlazer/dTOPO/CAT #38
pcDNA6.2/cGeneBlazer/dTOPO/CAT #48
pcDNA6/CAT (unfused CAT control)
pcDNA/GW-53/CAT (N-terminally fused GFP-CAT control)
pcDNA5/FRT/CAT/V5-His (C-terminally fused V5-His-CAT control)

Transfection cocktails were made with each of the vectors as follows:

| | 1X | 40X |
|---|---|---|
| OPTI-MEM | 50 μl | 2 ml |
| Lipofectamine 2000 | 1.5 μl | 60 μl |
| OPTI-MEM | 50 μl | |
| DNA | 0.5 μg | |

In addition to the above listed plasmids 100 ng of pcDNA5/FRT/Luciferase was co-transfected with each of the above listed plasmids as an internal control for determining transfection efficiency. Lipofectamine 2000 was added to the OPTI-MEM and allowed to equilibrate for 5 minutes at room temperature. Each of the DNAs was added to a separate tube of OPTI-MEM. Duplicates were not set up as a master mix, but rather, individually. After the 5-minute incubation, 50 μl of the LF2K in OPTI-MEM was added to each tube containing DNA and allowed to complex for 20 minutes at room temperature. Upon completion of the incubation, 100 μl of lipid/DNA complex was added to each well. Twenty-four hours after transfection, the media was aspirated from the wells, and the cells from each well were lysed with 100 μl of IX IGE PAL CA-630 lysis buffer (Sigma) with Complete Protease Inhibitor (Roche, 50× in H₂O) and Pepstatin (Roche, 1000× in EtOH). Lysates were harvested into 1.5 ml eppendorf tubes and centrifuged for 2 minutes at maximum speed. Cleared lysates were transferred to new tubes. During assays, lysates were kept on ice and then stored at −80° C.

Protein Assay

In a 96 well U-bottom flexible polyvinyl chloride plate (Falcon Cat. No. 35-3911) cell lysates were diluted 1:10 in H₂O (9 μL of H₂O and 1 μL of lysate). In duplicate, 10 μl of BSA standard curve was added to the plate (1000 μg/ml serial diluted 1:2 down to 15.625 μg/ml). One hundred and ninety microliters Bradford reagent were added to the μl of diluted lysates and standard curve (1:5 dilution of BioRad Protein Assay Solution Cat. No. 500-0006, 1 ml Solution and 4 ml H₂O). Using the plate reader and SoftMaxPro software, the endpoint wavelength was read at 595 and reduced numbers were displayed using the 4-parameter fit for the graph.

Western Blotting and Immuno-Detection Analysis

Samples run on the western blot gel included 15 μg of lysate, 4 μl of 4× NuPage Sample Buffer containing 0.4 μl volume of 2-Mercaptoethanol, and H₂O to 20 μl. Samples were heated at 70° C. for 10 minutes (with vortexing and centrifugation throughout) prior to loading 20 μl on a 4-12% NuPage Bis-Tris gel. The following controls were also added to the gel: 5 μl of Magic Mark, and 10 μl of See Blue Plus 2. 1× NuPage MOPS SDS Sample Running Buffer was used. Five hundred microliters of NuPage Antioxidant was added to the sample running buffer in the “inner core”. The gel was run for approximately 50 minutes at 200 volts.

NuPage Transfer Buffer was prepared with 20% methanol. One milliliter of Antioxidant was added to 1 liter of 1× NuPage Transfer Buffer. PVDF membranes were wetted in methanol, rinsed with H₂O, and then equilibrated in Transfer Buffer. Proteins from the gels were transferred to PVDF membrane for 90 minutes at 40 volts. Procedures from the NuPage Bis-Tris gel package insert were followed.

Following transfer, membranes were washed twice with 20 ml of H₂O and blocked for 30 minutes with the blocking solution in the anti-rabbit Western Breeze Chemiluminescent Kit. Diluted anti-CAT antibody (Sigma) to 10 μg/ml in primary antibody diluent for PVDF membranes from the anti-rabbit Western Breeze Chemiluminescent Kit. All the procedures recommended in the Western Breeze Chemiluminescent Kit were followed.

Results and Discussion

Construction of pENTR Spec-ccdB D-Topo

The Spectinomycin-ccdB cassette within pENTR Spec-ccdB D-Topo allows for Topo adaptation of GATEWAY® vectors with either the Bael or the NotI/AscI methodology. It also carries the ccdB gene which has been shown to reduce the number of background clones seen during Topo cloning. The Spectinomycin gene is a selectable marker for the cassette and helps to maintain the genetic fidelity of the toxic ccdB gene. pENTR Spec-ccdB D-Topo (FIG. 26) was verified by its restriction endonuclease digestion pattern and DNA sequence analysis.

The Spec-ccdB cassette allows for Topo adaptation with the NotI/AscI sites to generate D-Topo, Blunt-Topo or TA Topo vectors however the Bael sites are designed specifically to generate D-Topo vectors. The cassette is also movable to any Destination vector using a LR CLONASE™ reaction.

Construction of pcDNA6.2/nGeneBlazer-GW/D.3 and pcDNA6.2/cGeneBlazer-GW/D.3

pcDNA6.2/nGeneBlazer-GW/D.3 and pcDNA6.2/cGeneBlazer-GW/D.3 were constructed by moving the spec-ccdB cassette into either pcDNA6.2/nGeneBlazer-DEST or pcDNA6.2/cGeneBlazer-DEST using a LR CLONASE™ reaction. The efficiency of this transfer was 100% as determined by restriction endonuclease digestion profiles and DNA sequence analysis. Both the Bael and NotI/AscI Topo adaptation protocols were performed and their Topo cloning efficiencies are seen below. Both protocols generated a total colony count that exceeded 1800 colonies per Topo cloning reaction.

| | | | % |
|---|---|---|---|
| Topo Vector | Colonies/reaction | Background | Back-ground |
| pcDNA6.2/nGeneBlazer- | 7002 | 177 | 2.5 |
| GW/D.3 (BaeI) | | | |
| pcDNA6.2/cGeneBlazer- | 6660 | 207 | 3.1 |
| GW/D.3 (BaeI) | | | |
| pcDNA6.2/nGeneBlazer- | 10404 | 96 | 0.9 |
| GW/D.3 (NotI/AscI) | | | |
| pcDNA6.2/nGeneBlazer- | 12438 | 150 | 1.1 |
| GW/D.3 (NotI/AscI) | | | |

The DTopo 750 bp PCR amplified fragment was used to assess the Topo cloning efficiency of the Topo-adapted vectors. Background colony numbers were generated from reactions containing no PCR product. The transformation competency of the TOP10 cells was determined to be 10¹⁰ cfu/ug.

Topo Adaptation Efficiency Assay

Plasmid DNA of 10 colonies from each of the Topo reactions described above were isolated to determine the presence and the orientation of the cloned 750 bp fragment. All clones isolated demonstrate that the 750 bp PCR amplified fragment was cloned and in the correct orientation except for one clone, which showed a clone bearing no insert. DNA sequence analysis confirmed that the junctions of the Topo cloned DNA ends were the predicted sequences.

Cloning efficiency of Topo vectors adapted with the Bael Topo adaptation protocol was assessed. The 750 bp fragment was Topo cloned into Topo charged pcDNA6.2/cGeneBlazer-GW/D.3 and plasmid DNA isolated from 10 of the colonies generated was digested with AvaI and showed the predicted digestion profile. The AvaI digest of plasmid DNA from positive clones will yield 3.9 kb and 2.7 kb DNA fragments. The 750 bp fragment was Topo cloned into Topo charged pcDNA6.2/nGeneBlazer-GW/D.3 and plasmid DNA isolated from 10 of the colonies generated was digested with AvaI. The AvaI digest of plasmid DNA from positive clones will yield 4.7 kb and 1.8 kb DNA fragments. All clones analyzed showed the predicted digestion profile.

Cloning efficiency of Topo vectors adapted with the NotI/AscI Topo adaptation protocol was assessed. The 750 bp fragment was Topo cloned into Topo charged pcDNA6.2/cGeneBlazer-GW/D.3 and plasmid DNA isolated from 10 of the colonies generated was digested with AvaI. The AvaI digest of plasmid DNA from positive clones will yield 3.9 kb and 2.7 kb DNA fragments. All clones analyzed showed the predicted digestion profile. The 750 bp fragment was Topo cloned into Topo charged pcDNA6.2/nGeneBlazer-GW/D.3 and plasmid DNA isolated from 10 of the colonies generated was digested with AvaI. The AvaI digest of plasmid DNA from positive clones will yield 4.7 kb and 1.8 kb DNA fragments. All but one of the clones showed the predicted digestion profile.

BP reactions with pcDNA6.2/nGeneBlazer-TopoCAT and pcDNA6.2/cGeneBlazer-TopoCAT

The GATEWAY® reaction allows for the transfer of ORFs from GATEWAY® expression vectors to Donor vectors to create Entry clones. To confirm that our Topo adapted vectors were functional in a BP reaction, pcDNA6.2/nGeneBlazer-TopoCAT and pcDNA6.2/cGeneBlazer-TopoCAT were used in BP reactions to transfer the CAT gene to pDONR221 creating pENTR-CAT clones. The colony counts from the BP reactions are seen below and demonstrate a highly efficient BP reaction. Restriction endonuclease digestion analysis of eight colonies from the BP reactions also demonstrated that the BP reaction has a 100% efficiency in transferring the CAT gene to pDONR221. The control reaction with no BP CLONASE™ added generated zero colonies.

| BP Reaction | Colonies/reaction |
|---|---|
| pcDNA6.2/nGeneBlazer-TopoCAT/pDONR221 | 37950 |
| pcDNA6.2/cGeneBlazer-TopoCAT/pDONR221 | 34155 |

BP reaction were conducted with pDONR221 and pcDNA6.2/nGeneBlazer-TopoCAT or pcDNA6.2/cGeneBlazer-TopoCAT. Total colonies generated from BP reactions with CAT-β-lactamase fusion expression clones and pDONR221 were determined. The colony numbers are averages from 2 independent reactions. Plasmid DNA of 4 colonies from each of the BP reactions were digested with BsrGI. The BsrGI digestion of pENTR-CAT will yield 2.5 kb and 0.7 kb DNA fragments. All clones tested showed the correct digestion pattern.

Expression analysis of pcDNA6.2/nGeneBlazer-TopoCAT and pcDNA6.2/cGeneBlazer-TopoCAT

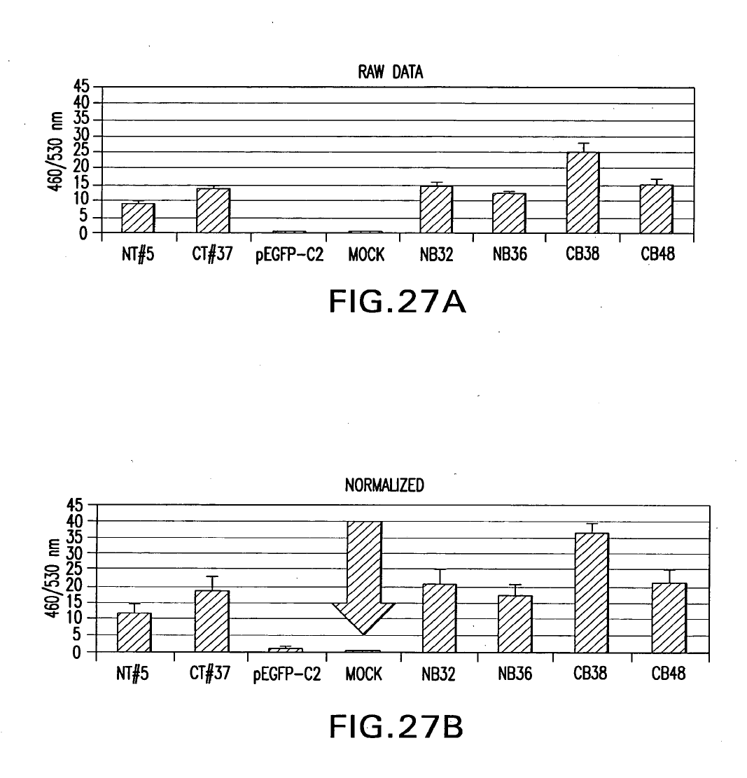

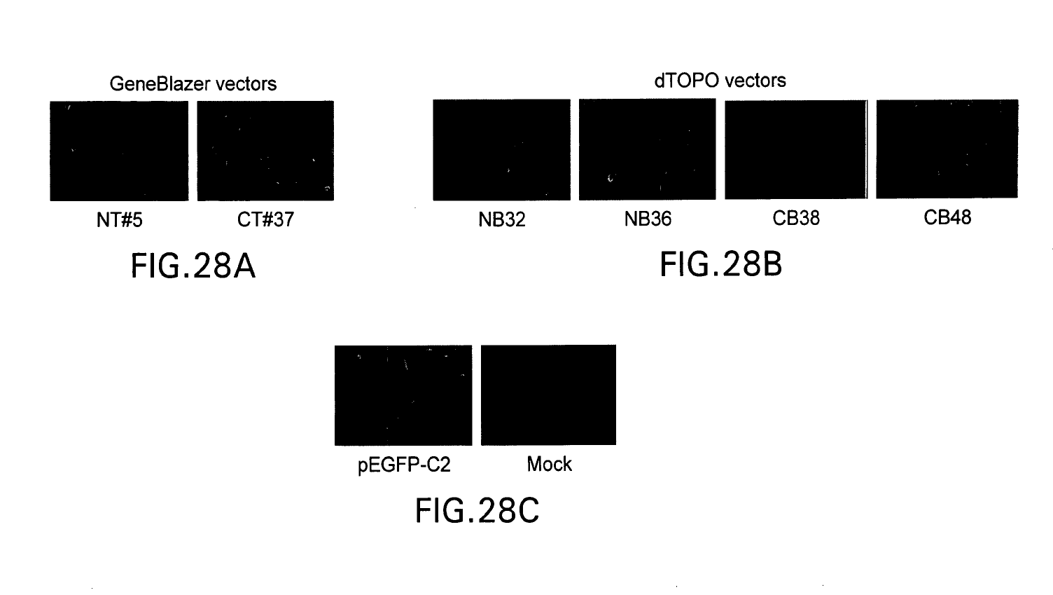

The expression data of pcDNA6.2/nGeneBlazer-TopoCAT and pcDNA6.2/cGeneBlazer-TopoCAT is shown in FIGS. 27, 28, and 29. In vivo detection data (FIGS. 27 and 28) indicate that CAT fused to either the N or C terminus of β-lactamase by Topo cloning produces an active β-lactamase fusion protein.

B-lactamase expression in GripTite 293 cell lines was assessed and is shown in FIG. 27. 48-hours post transfection, cells were treated with the CCF4-AM substrate. Graphed data was obtained using an endpoint read on the Molecular Devices Spectra Max Gemini XS plate reader at dual wavelengths of 460 nm and 530 nm. The left panel represents the raw data from the plate reader. The right panel data is normalized to the mock transfection being equal to one. NT#5: pcDNA6.2/nGeneBlazer-GW/lacZ, CT#37: pcDNA6.2/cGeneBlazer-GW/lacZ, NB32 and NB36: pcDNA6.2/nGeneBlazer-TopoCAT, CB38 & CB48: pcDNA6.2/nGeneBlazer-TopoCAT. The β-lactamase activity levels from the expression of pcDNA6.2/nGeneBlazer-TopoCAT and pcDNA6.2/cGeneBlazer-TopoCAT (FIG. 27, columns NB32, NB36, CB38 and CB48) are comparable to the β-lactamase activity levels from expression of pcDNA6.2/nGeneBlazer-GW/lacZ and pcDNA6.2/nGeneBlazer-GW/lacZ (FIG. 27, columns NT#5 and CT#37) suggesting that the methodology of cloning of β-lactamase fusions does not affect the activity of the expressed β-lactamase fusions and that β-lactamase fused to either CAT or lacZ does affect the activity of the expressed β-lactamase Cat or lacZ fusions.

GripTite 293 cell lines demonstrating β-lactamase expression were prepared and digital images were prepared and are shown in FIG. 28. Cells were treated with the CCF4-AM substrate 48-hours post transfection and digital images were taken at 77 mm with a 1.5 second exposure (Heidi Welchin, NB#5461, pages 169-171). NT#5: pcDNA6.2/nGeneBlazer-GW/lacZ, CT#37: pcDNA6.2/cGeneBlazer-GW/lacZ, NB32 & NB36: pcDNA6.2/nGeneBlazer-TopoCAT, CB38 & CB48: pcDNA6.2/nGeneBlazer-TopoCAT, pEGFP-C2: transfection control, Mock: no plasmid DNA.

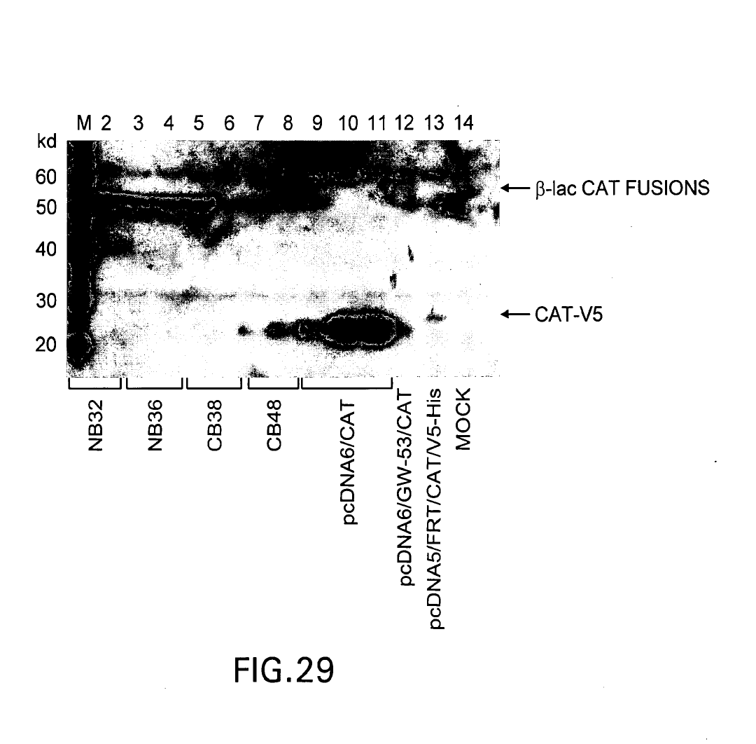

Expression of CAT in GeneBlazer dTOPO plasmids was assessed by Western blot and the results are shown in FIG. 29. COS-7 cells were transiently transfected with pcDNA6.2/nGeneBlazer/dTOPO/CAT clone NB32 (lanes 2-3), pcDNA6.2/nGeneBlazer/dTOPO/CAT clone NB36 (lanes 4-5), pcDNA6.2/cGeneBlazer/dTOPO/CAT clone CB38 (lanes 6-7) pcDNA6.2/cGeneBlazer/dTOPO/CAT clone CB48 (lanes 8-9), pcDNA6/CAT (lanes 10-11), pcDNA/GW-53/CAT (lane 12), pcDNA5/FRT/CAT/V5-His (lane 13), untransfected control (lane 14), Cell lysates were analyzed by western blot with anti-CAT antiserum. M=Magic Mark molecular weight marker. Immuno-detection using anti-CAT antibodies demonstrated the presence of CAT-β-lactamase fusions (FIG. 29, lanes 2 to 9). CAT fused at its C-terminus with β-lactamase seems to be expressed at lower levels (FIG. 29, lanes 6 to 9) compared to CAT fused at its N-terminus to β-lactamase (FIG. 29, lanes 2 to 5). This is contrary to the measured β-lactamase activity levels of the CAT-α-lactamase fusions which indicate similar levels of β-lactamase activity for both CAT-α-lactamase fusions (FIG. 27, columns NB32, NB36, CB38 and CB48). There is no obvious explanation for this discrepancy other than the observation that the V5 tag also affects the detection or expression levels of CAT when fused to the C-terminus of CAT (FIG. 29, lane 13) compared to the expression of native CAT (FIG. 29, lane 10 and 11).

In conclusion, the Topo-charged pcDNA6.2/nGeneBlazer-GW/D.3 and pcDNA6.2/cGeneBlazer-GW/D.3 have met the key performance criteria. The Topo cloning efficiency with respect to the foreground and background colony numbers was greater than 90%, the cloning of an insert with directionality was shown to be 95% and the resulting clones from the Topo reaction were active in a BP reaction and produced β-lactamase fusion proteins with β-lactamase activities comparable to β-lactamase fusion proteins cloned by GATEWAY® recombination reactions.

### Example 8

### Summary

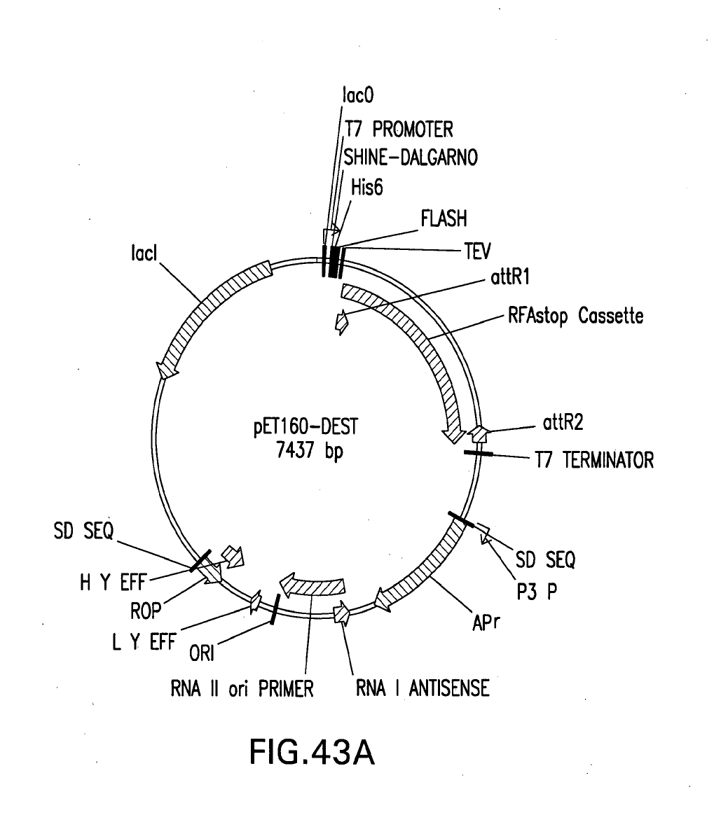

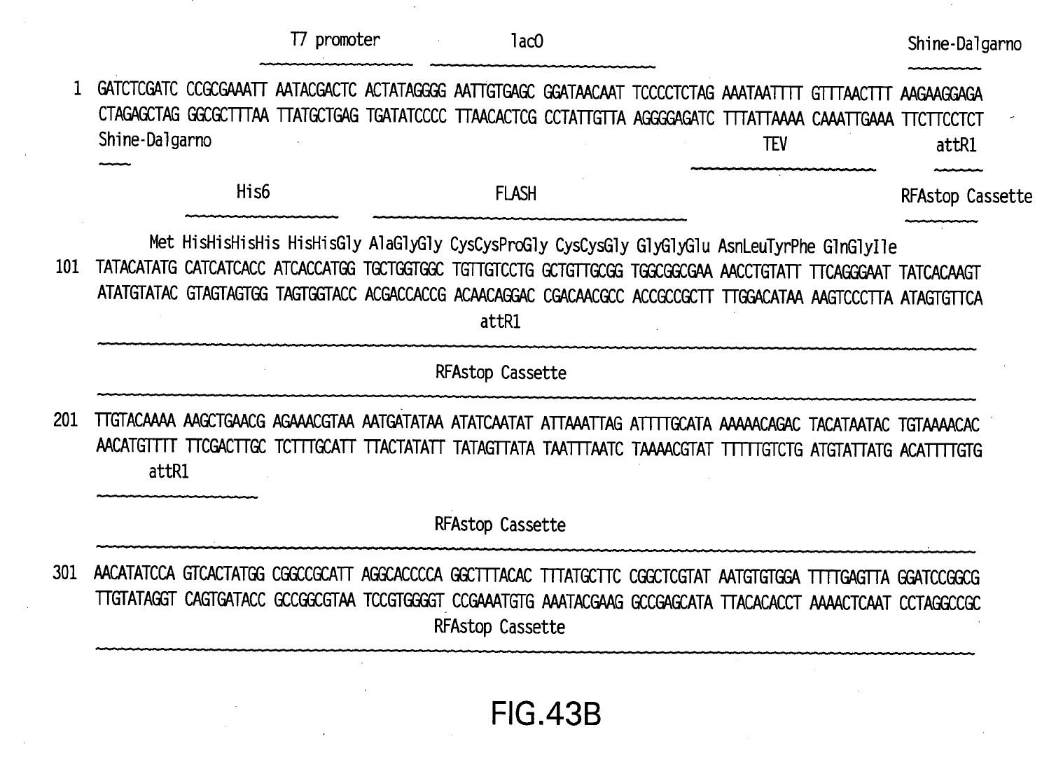

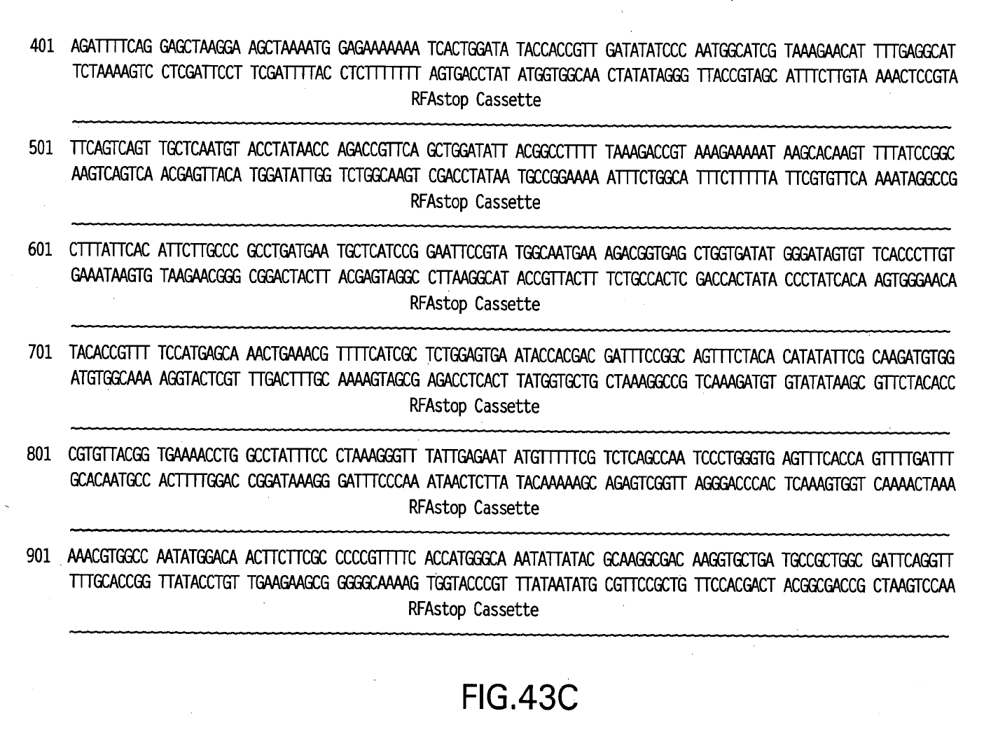

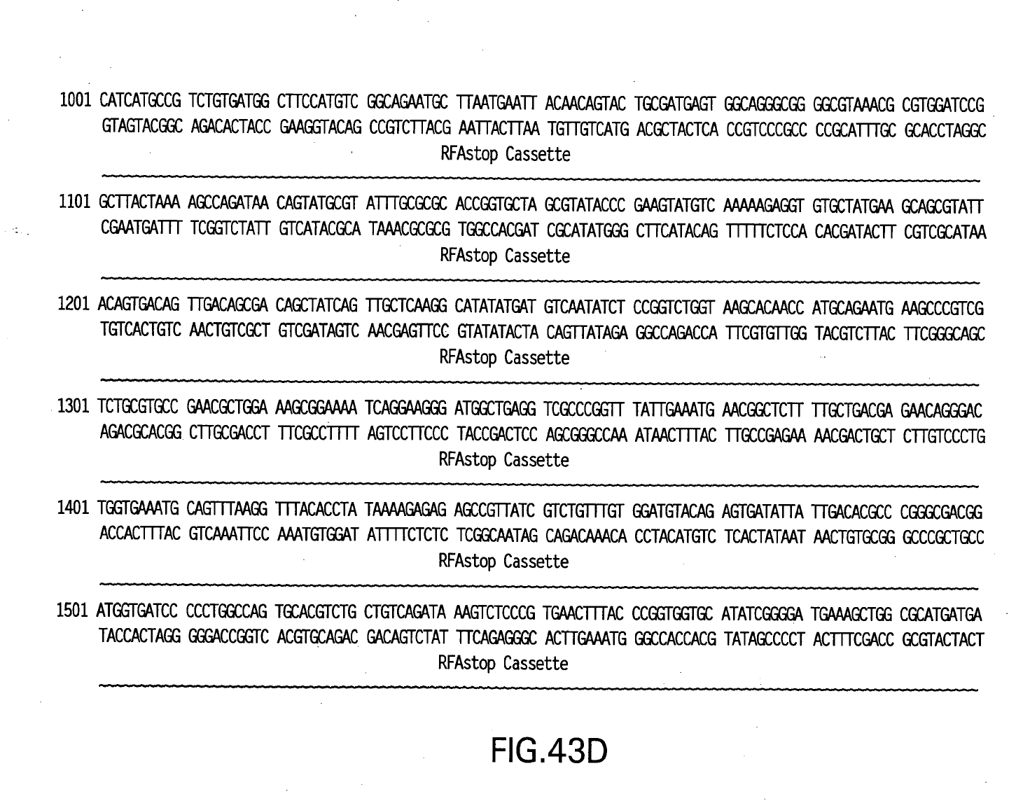

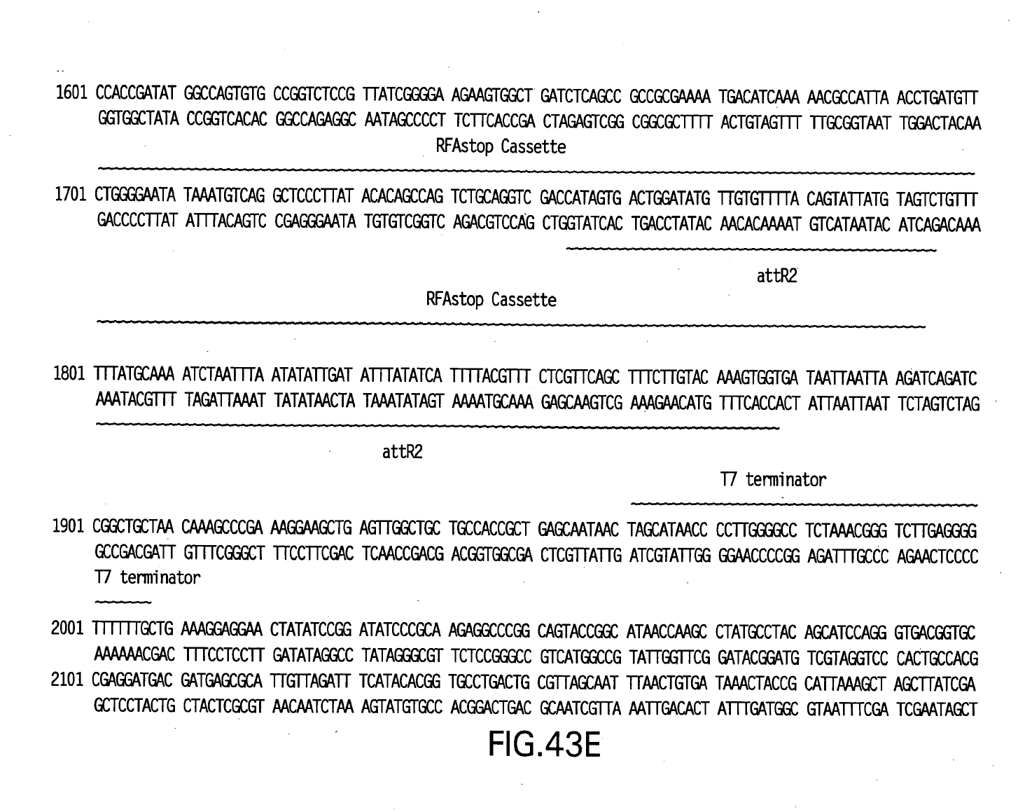

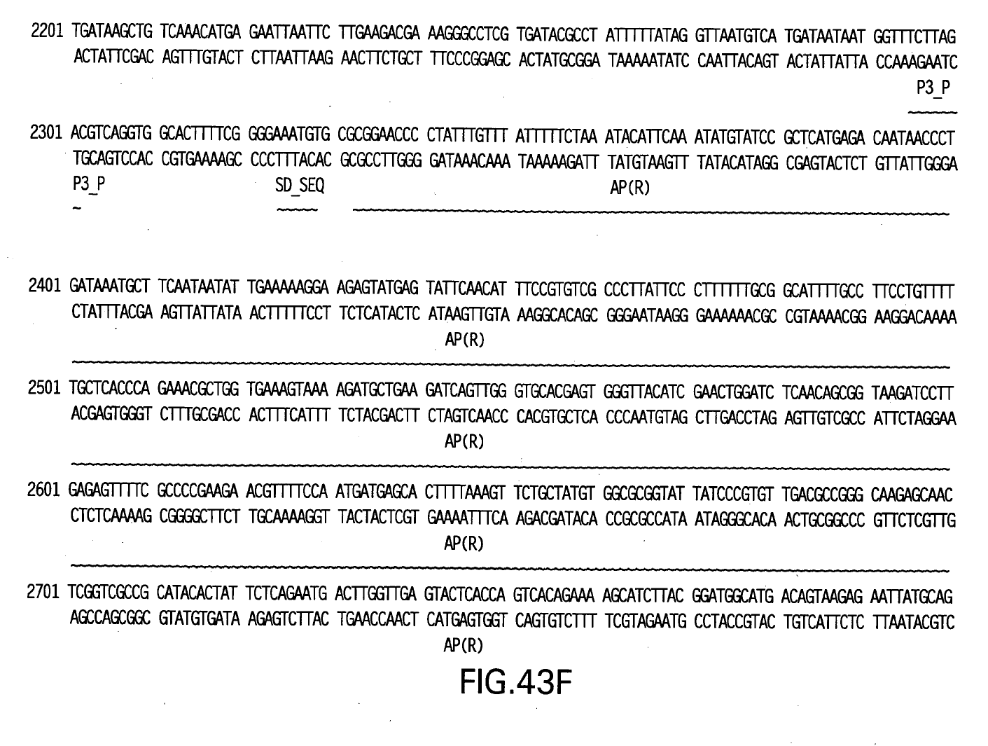

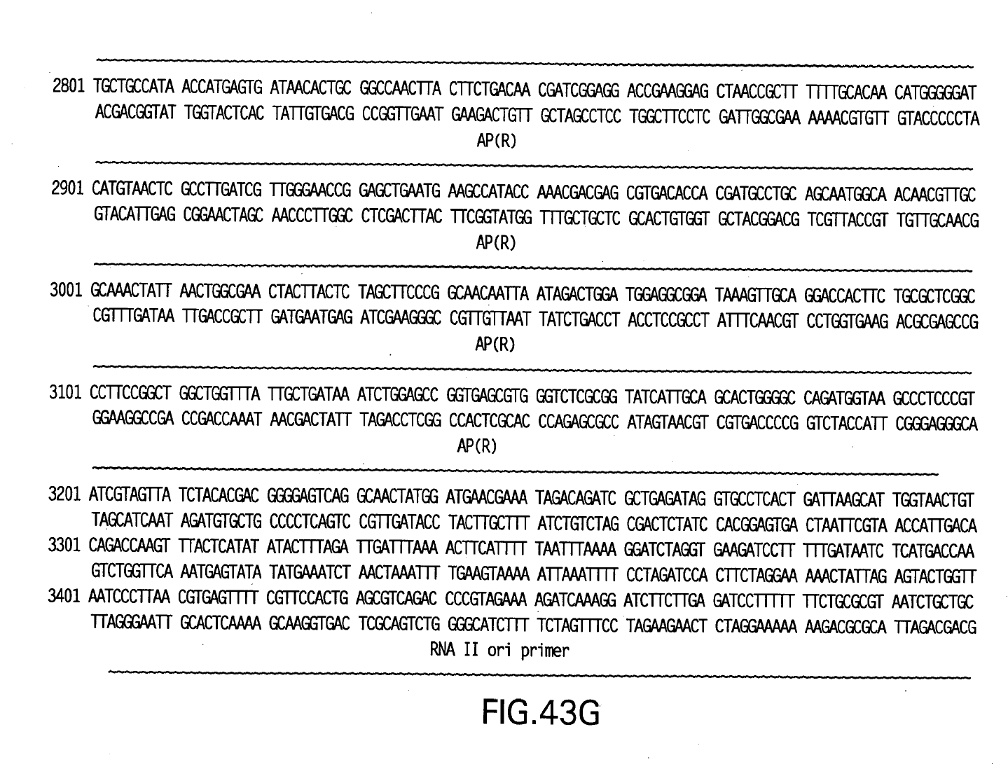

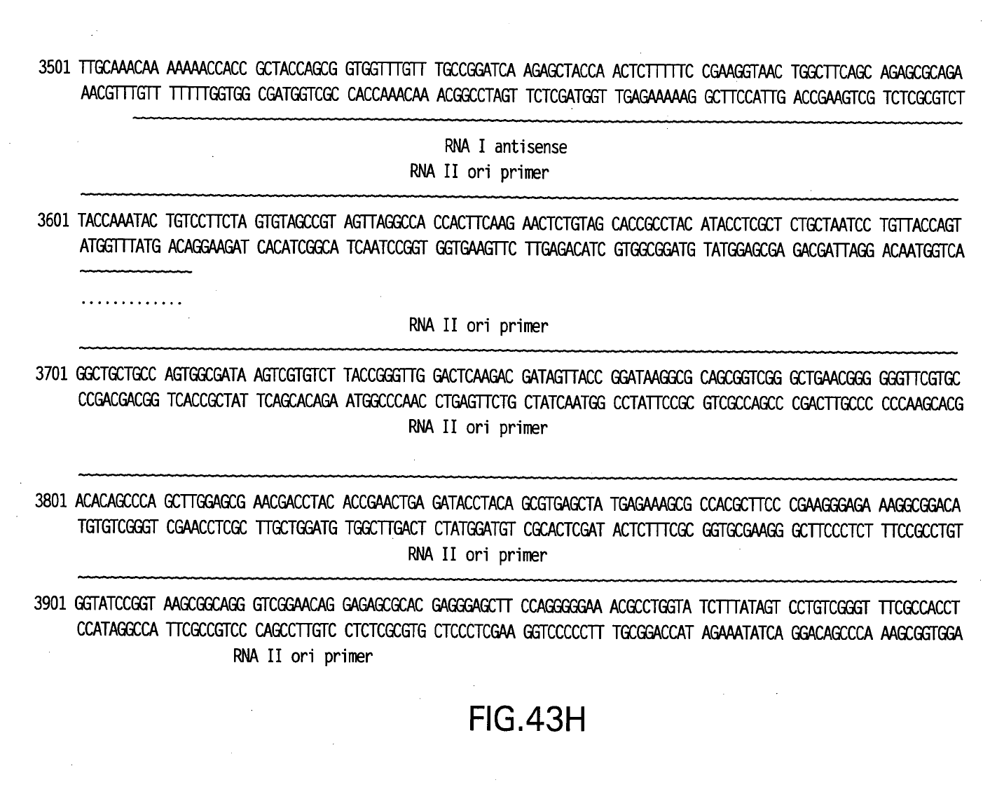

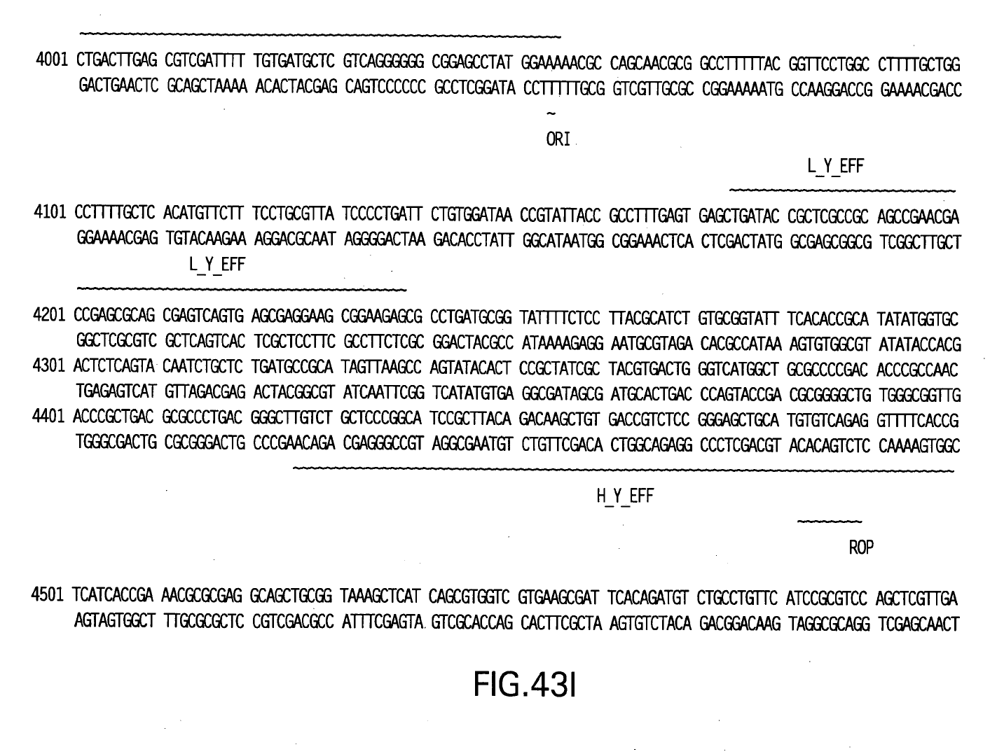

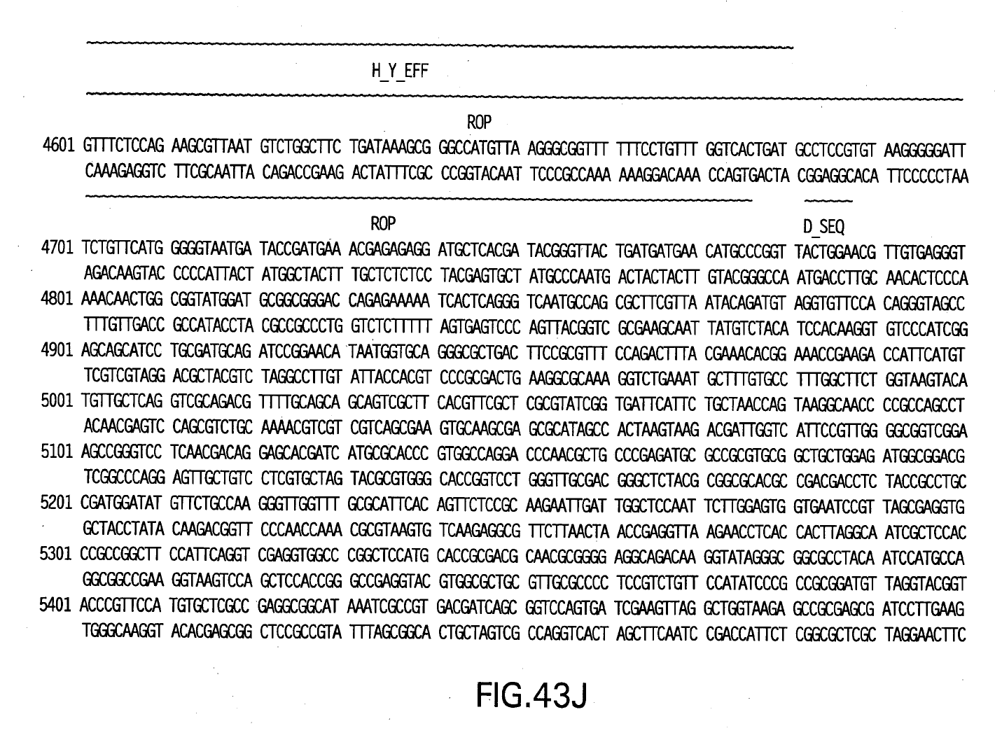

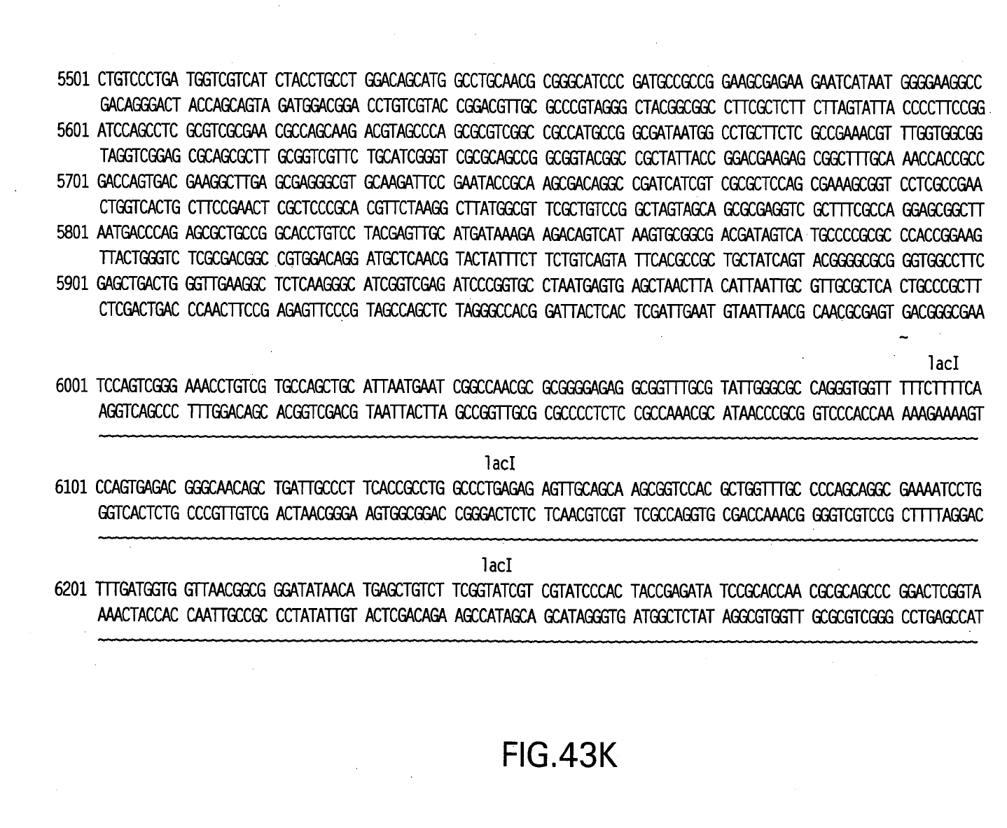

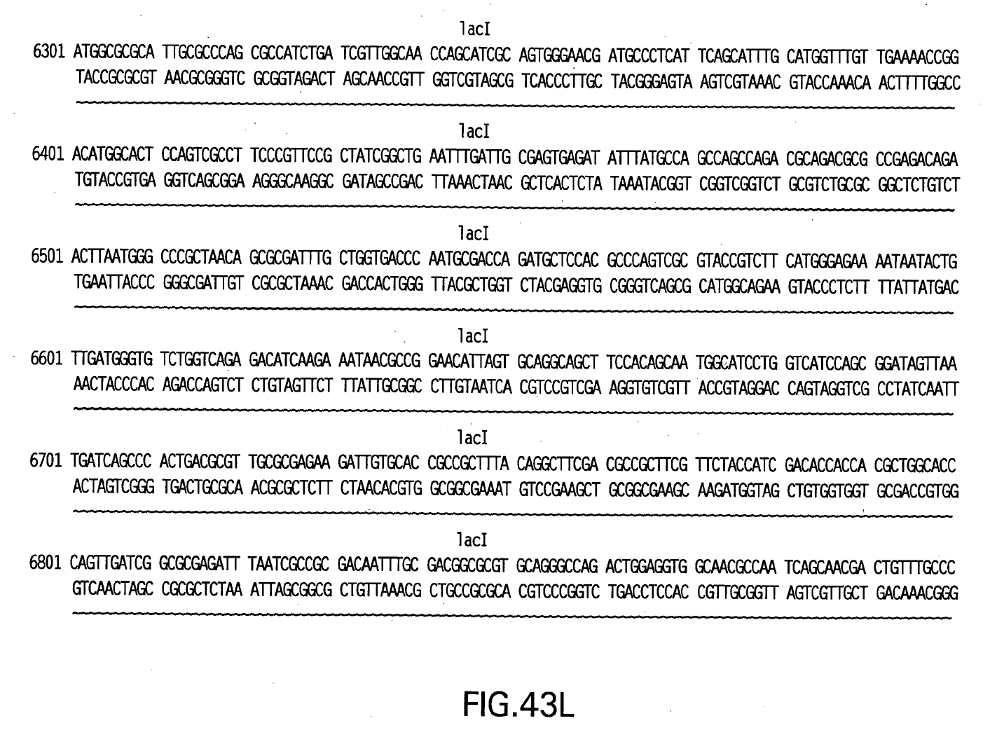

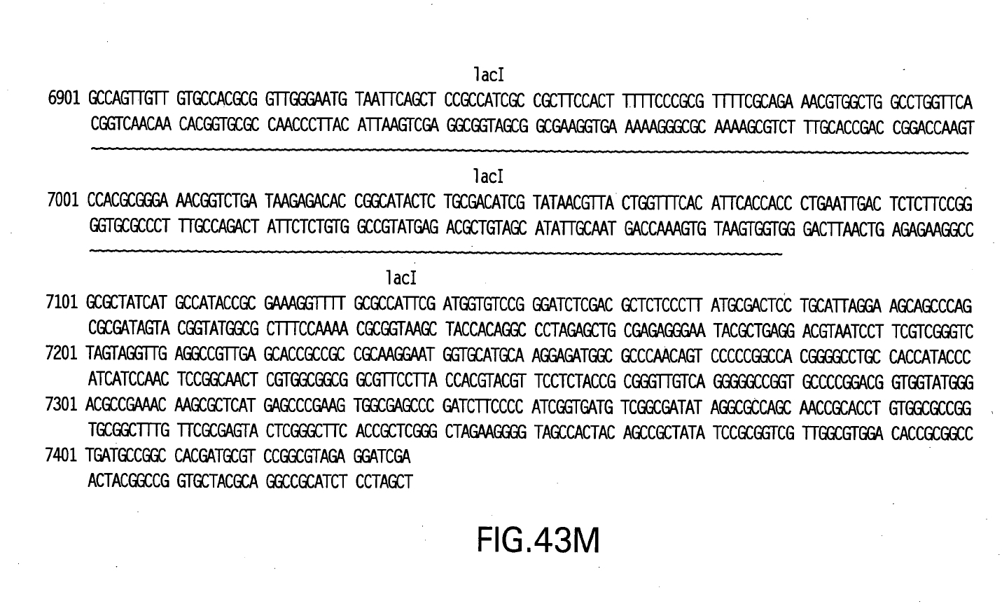

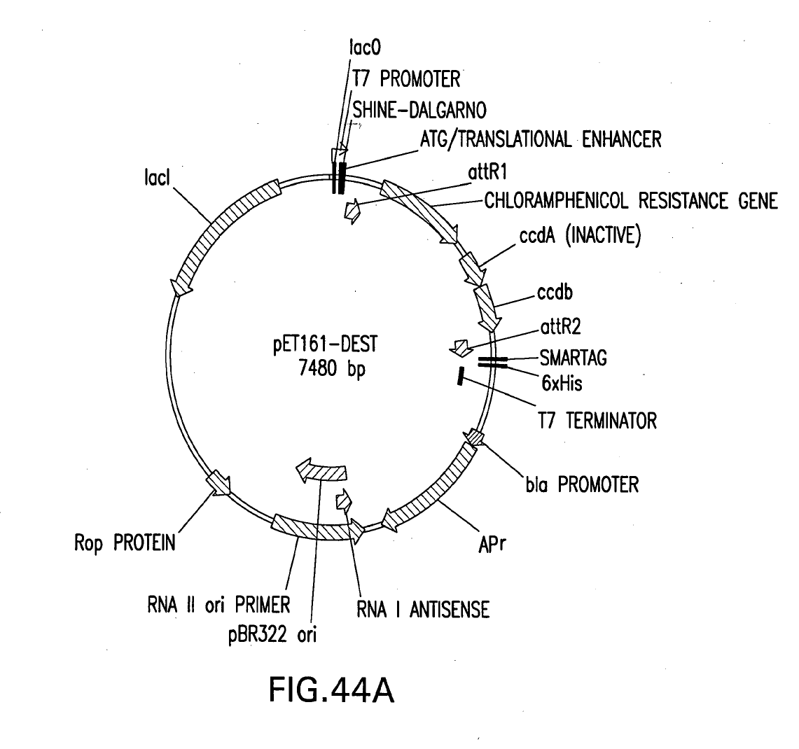

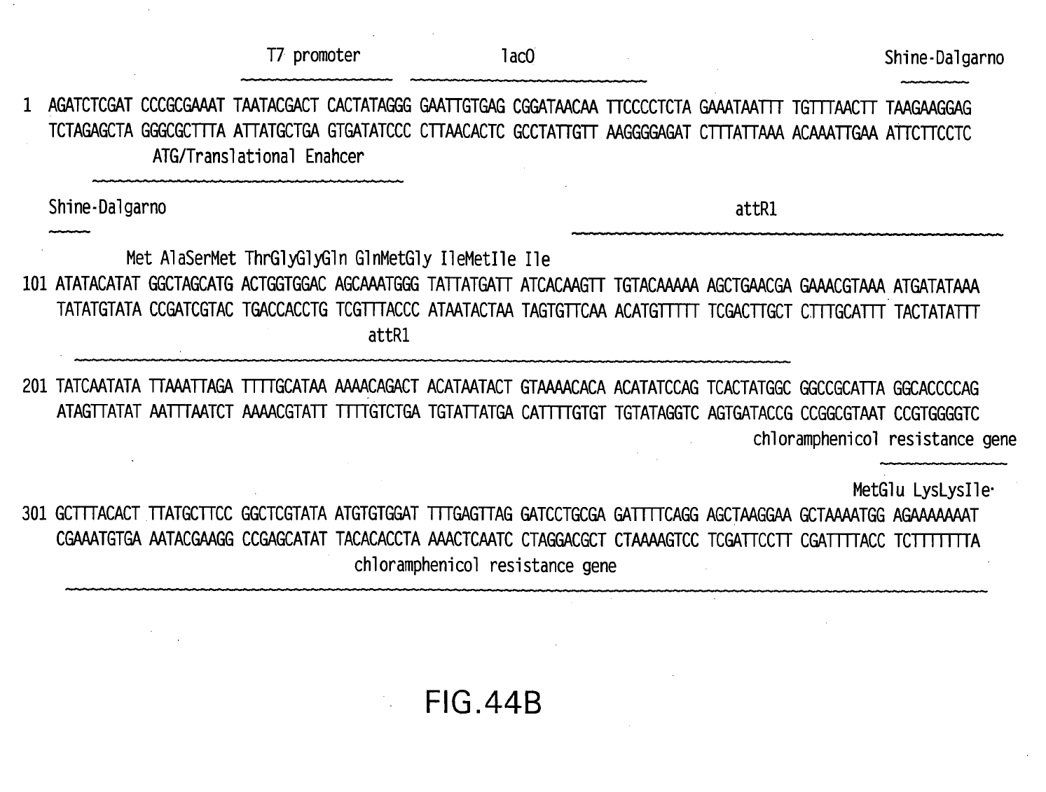

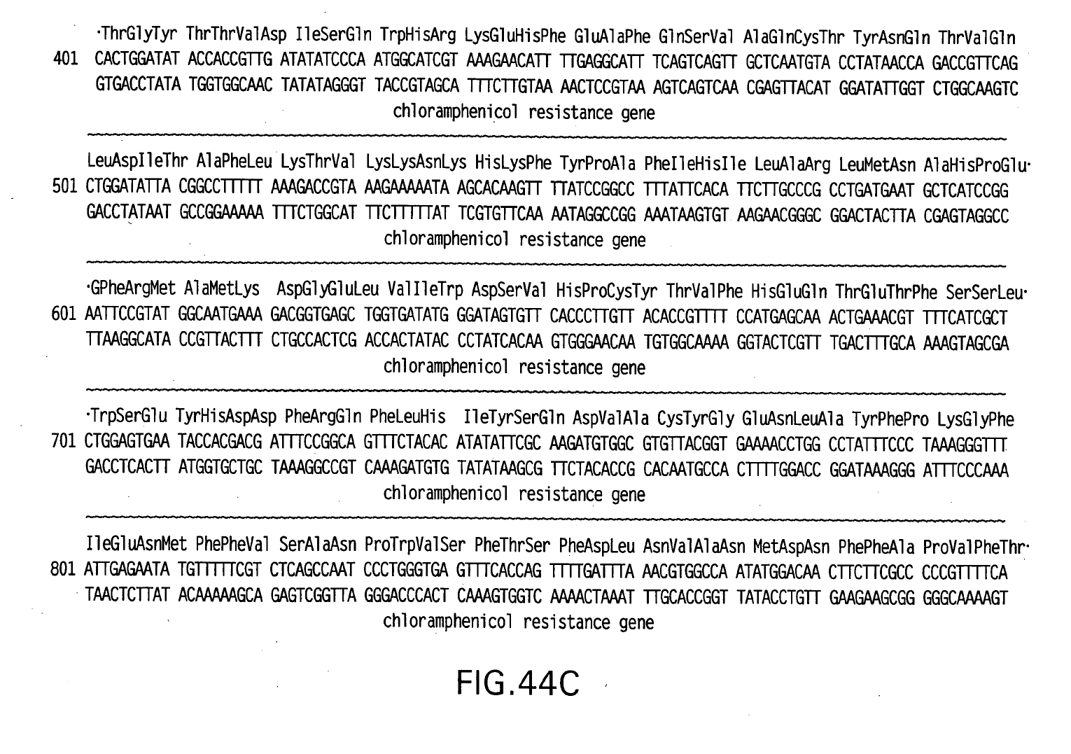

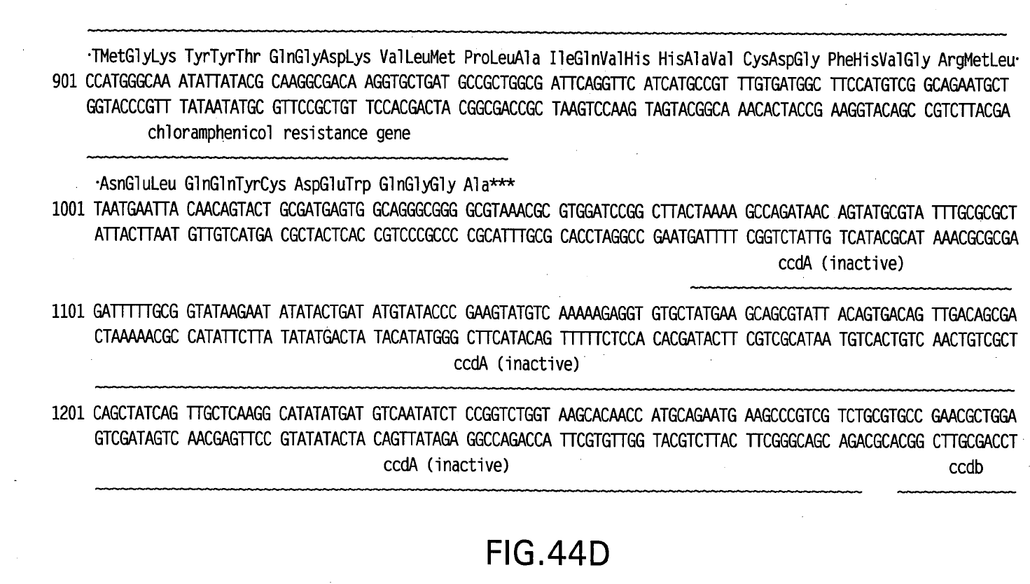

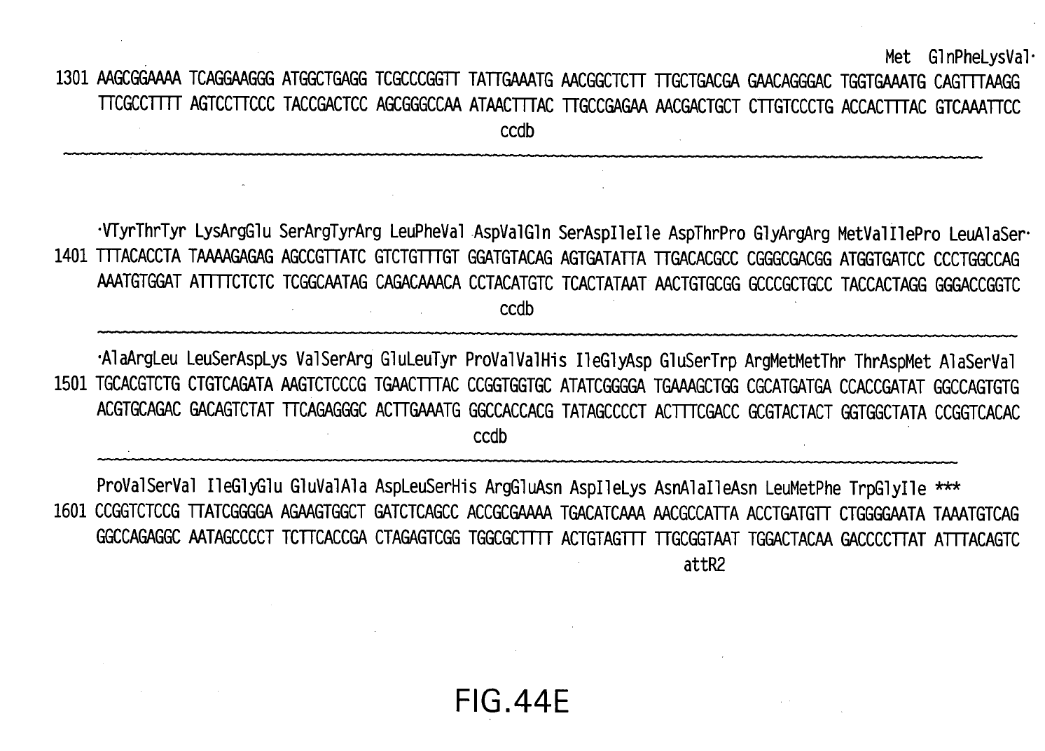

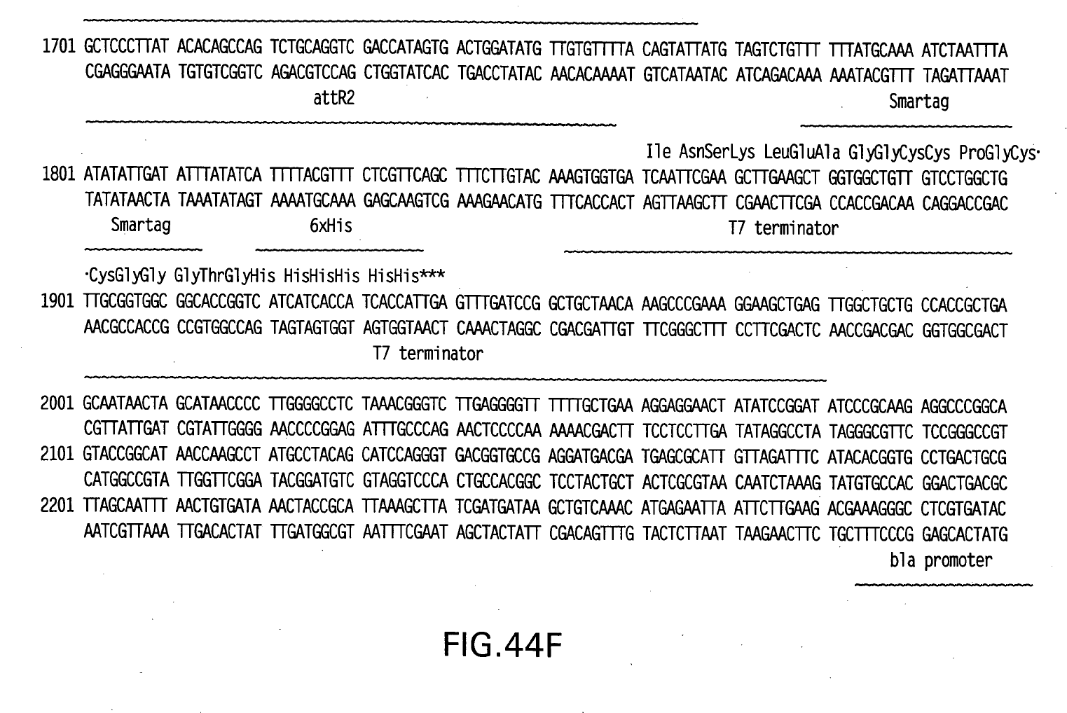

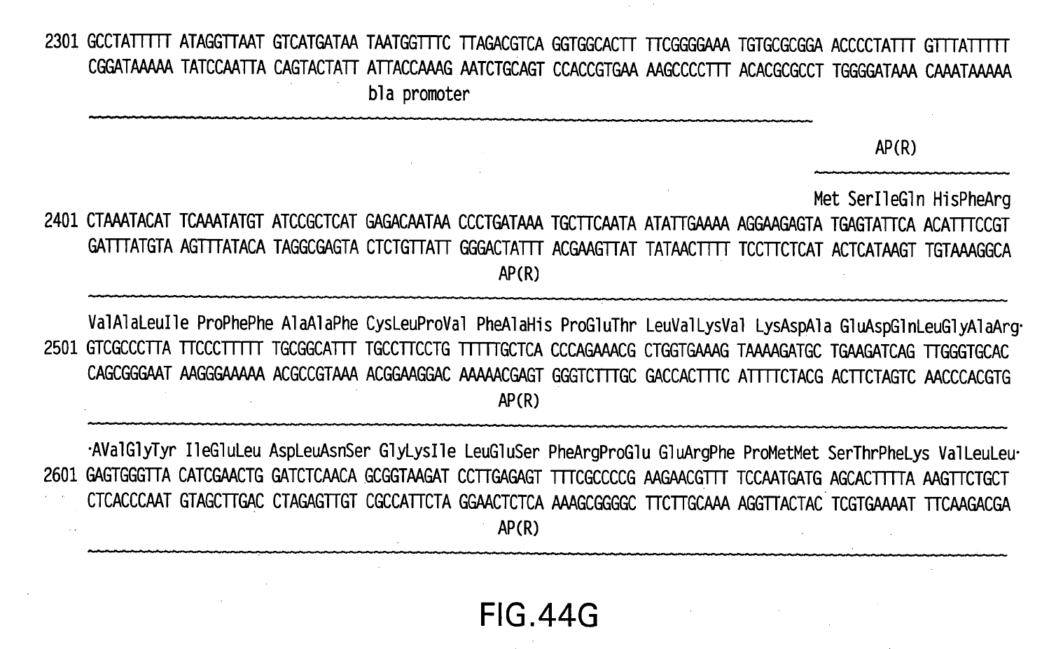

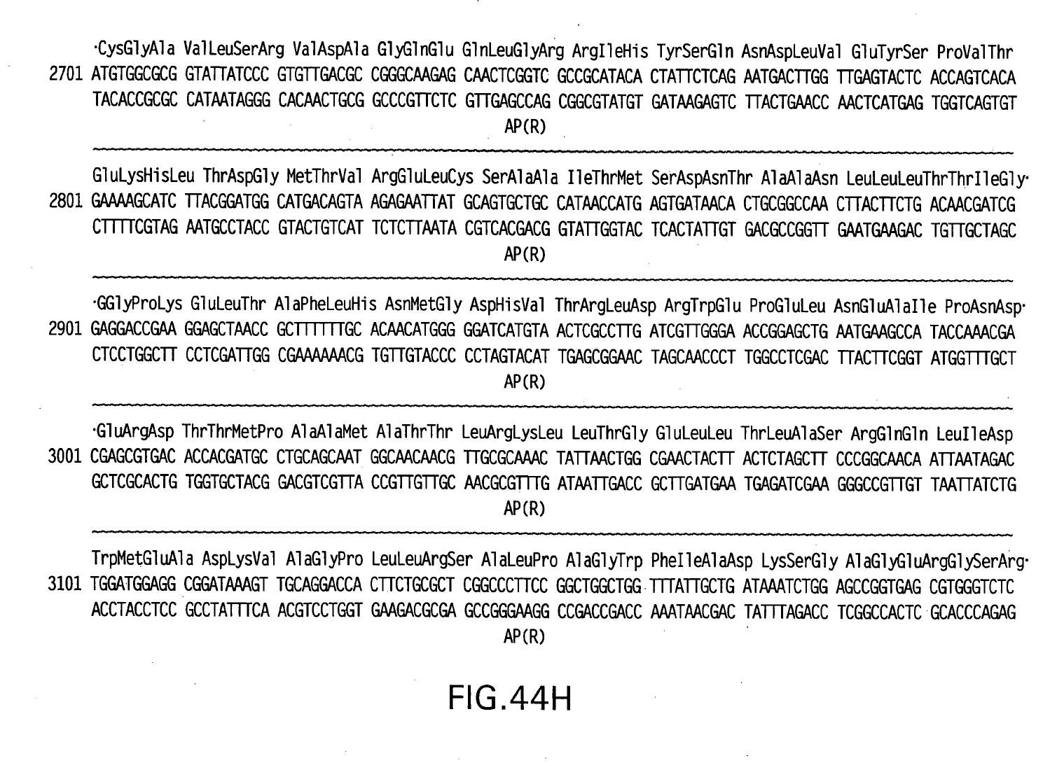

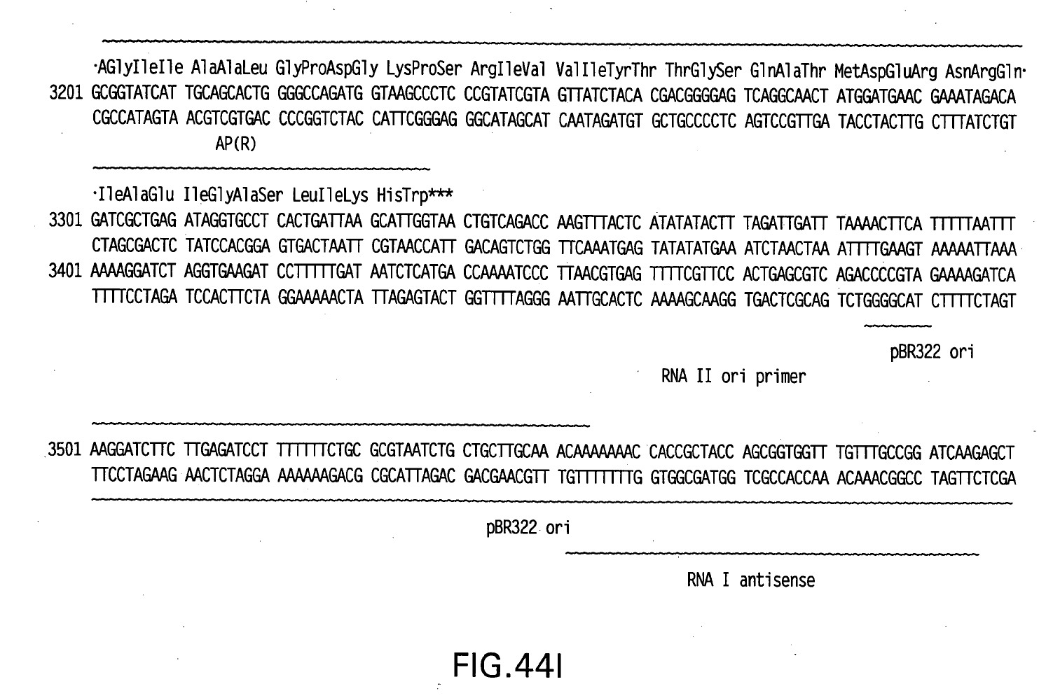

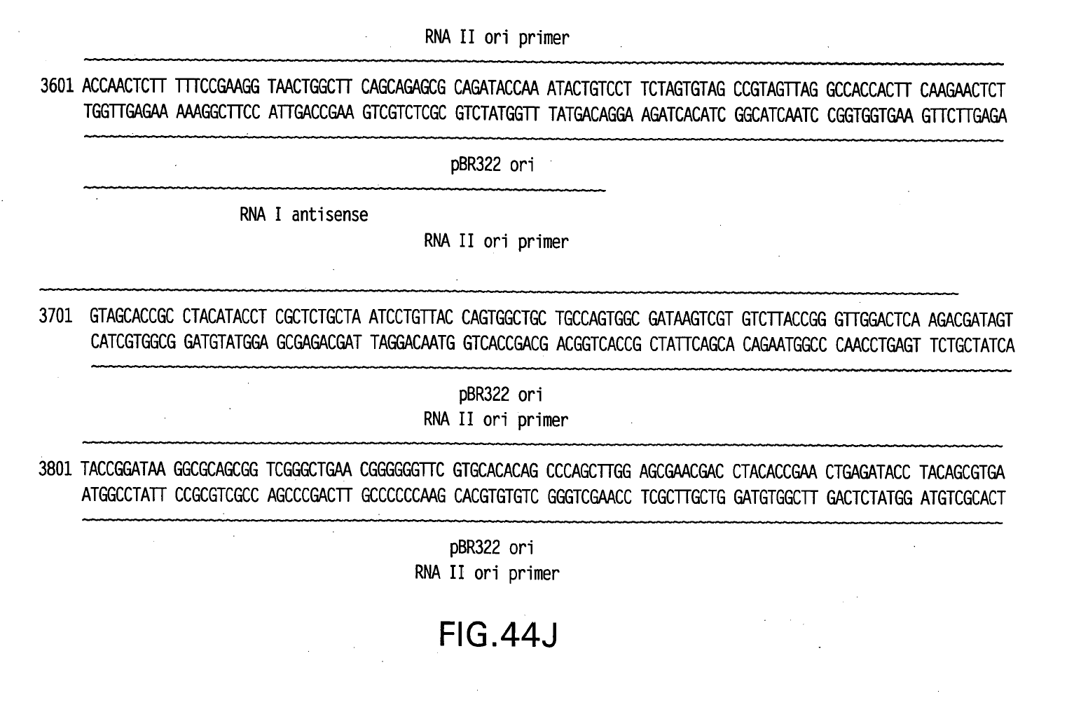

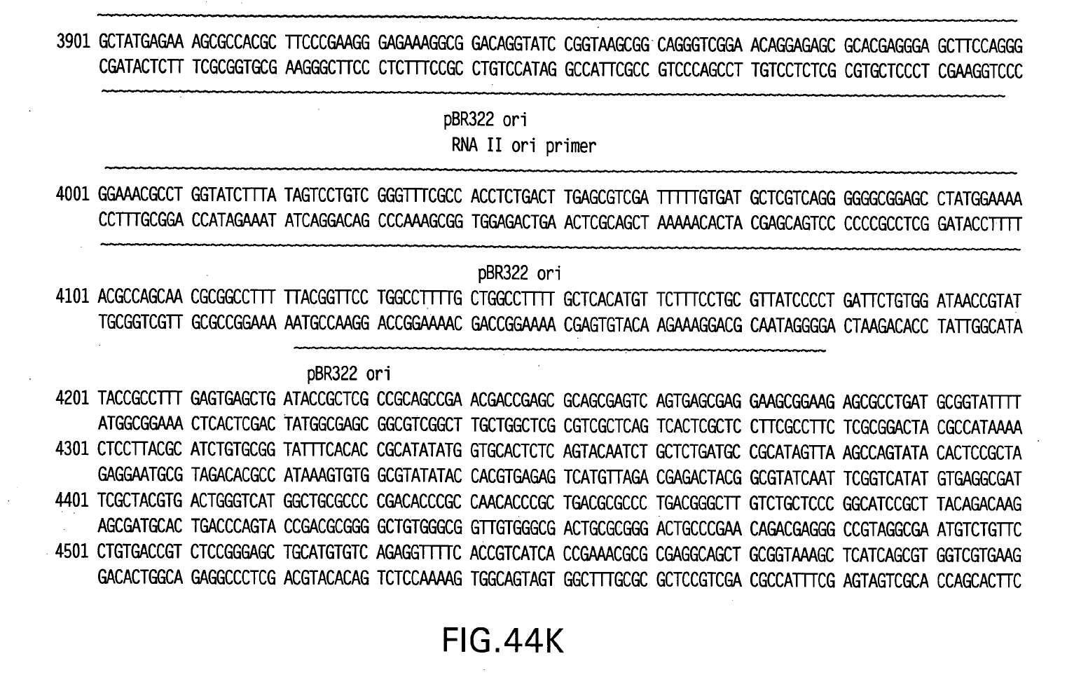

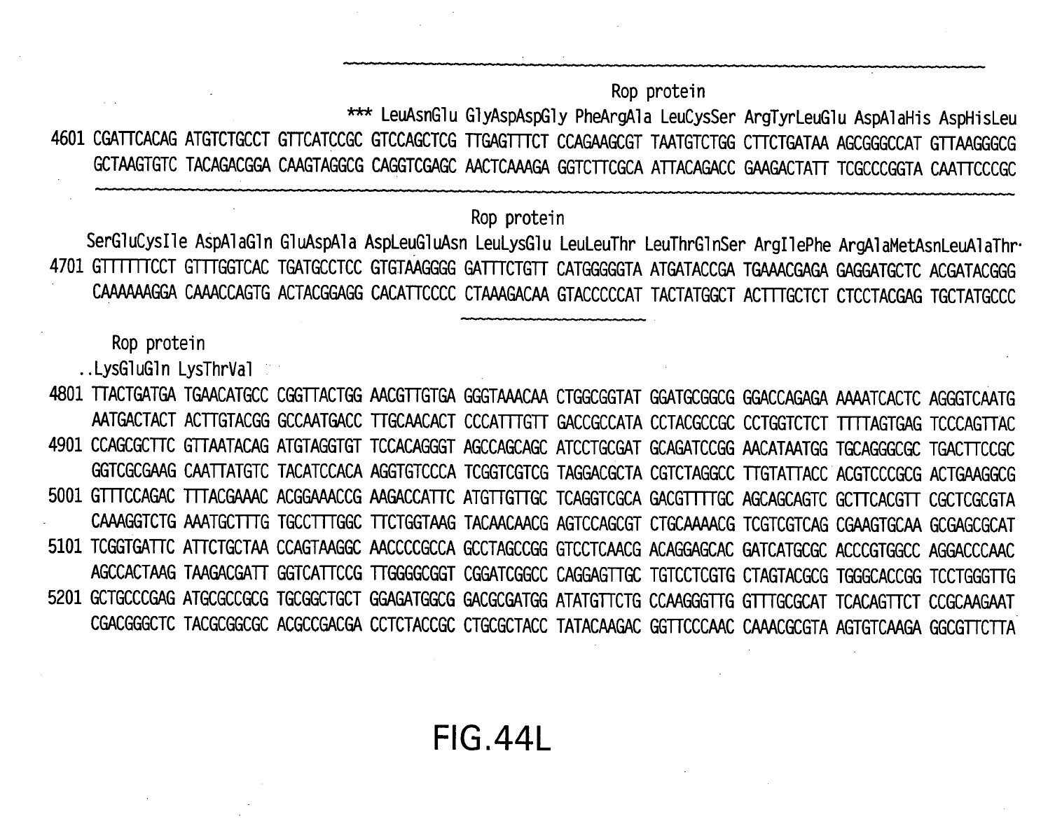

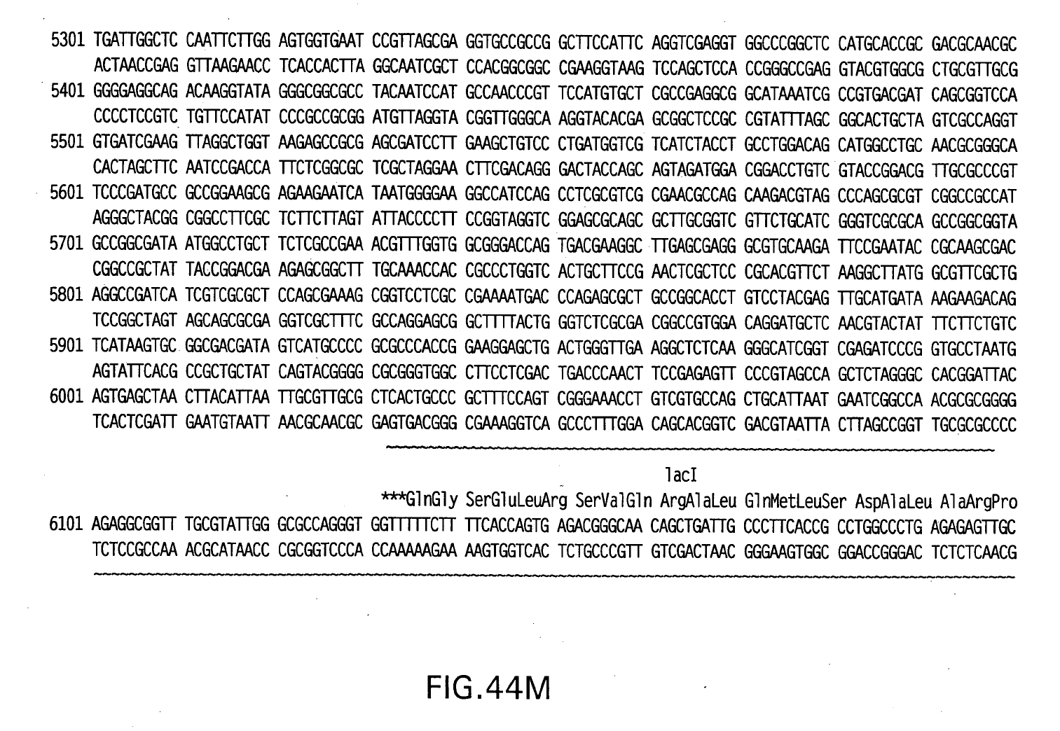

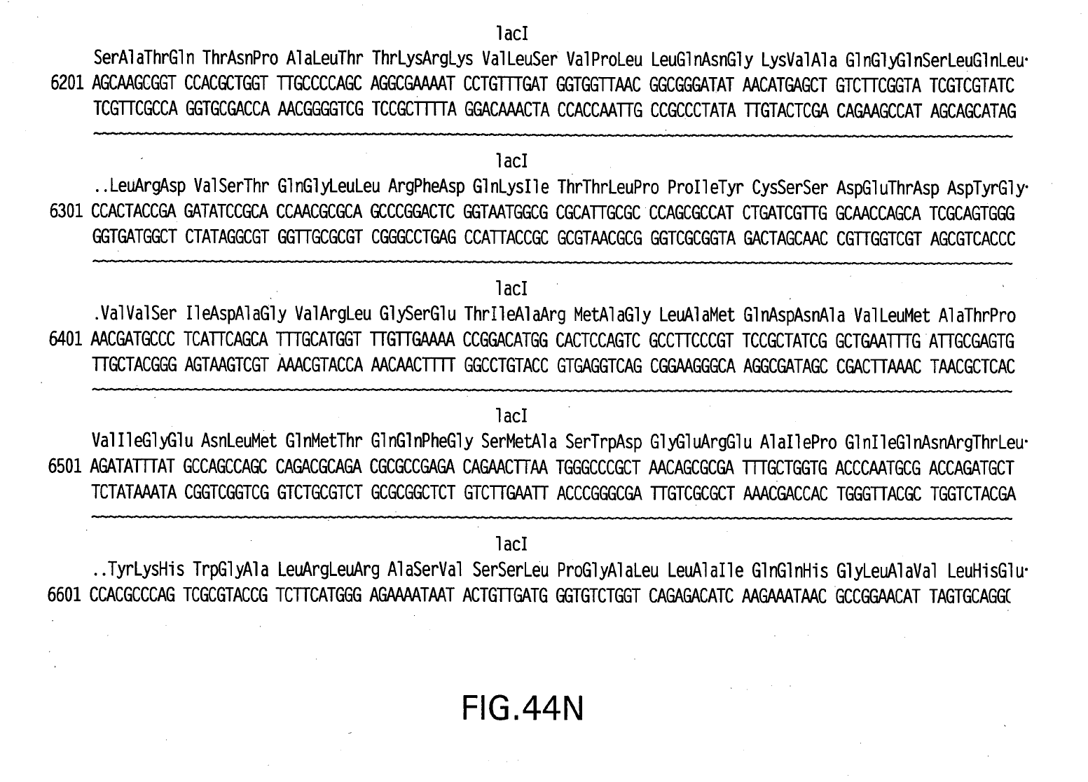

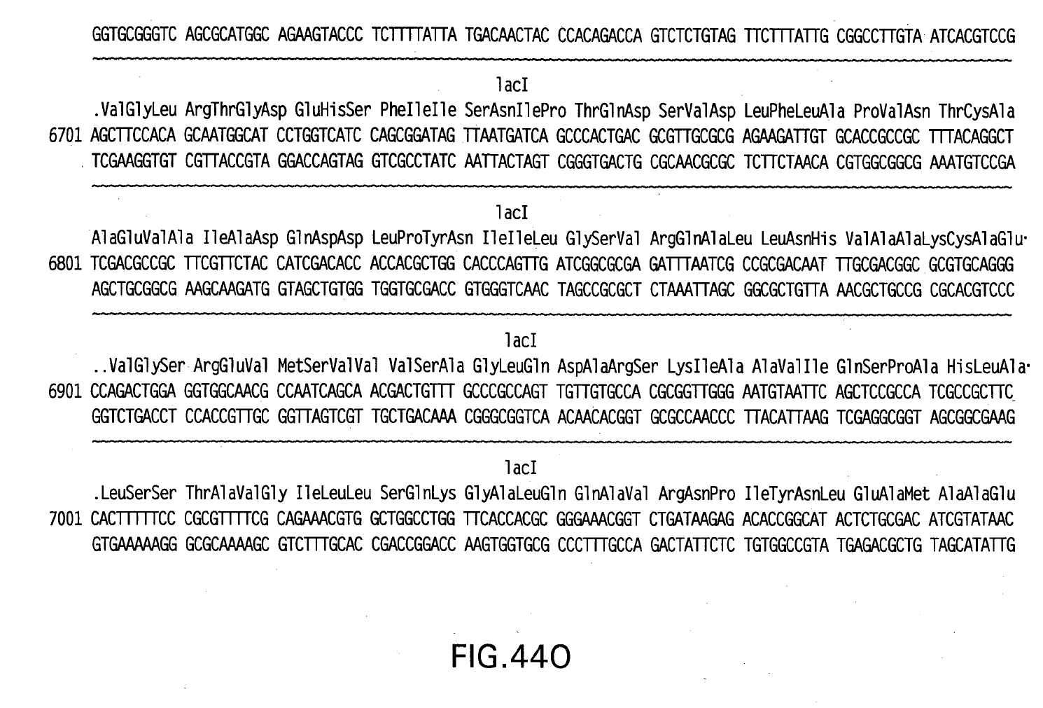

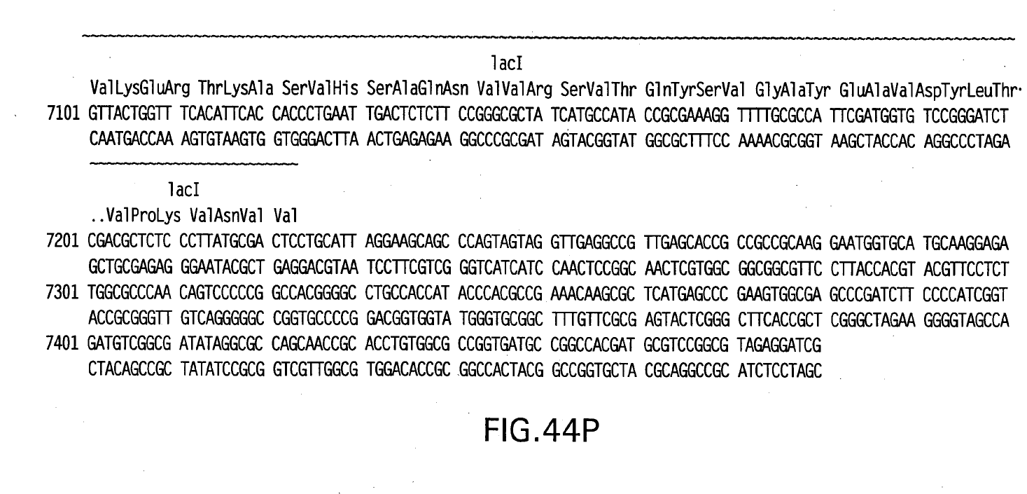

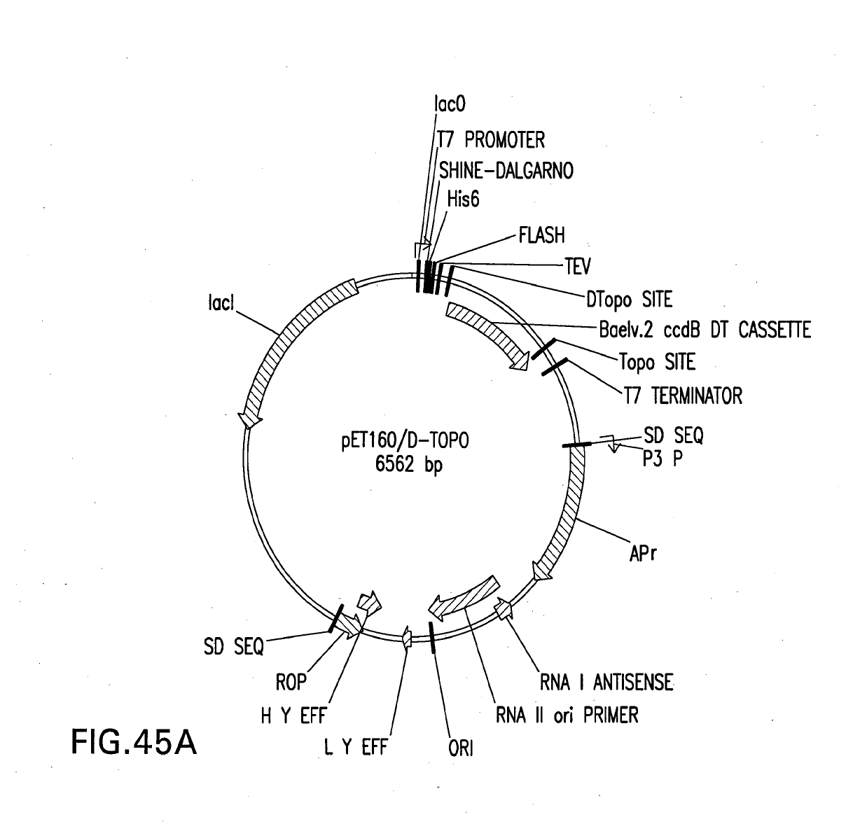

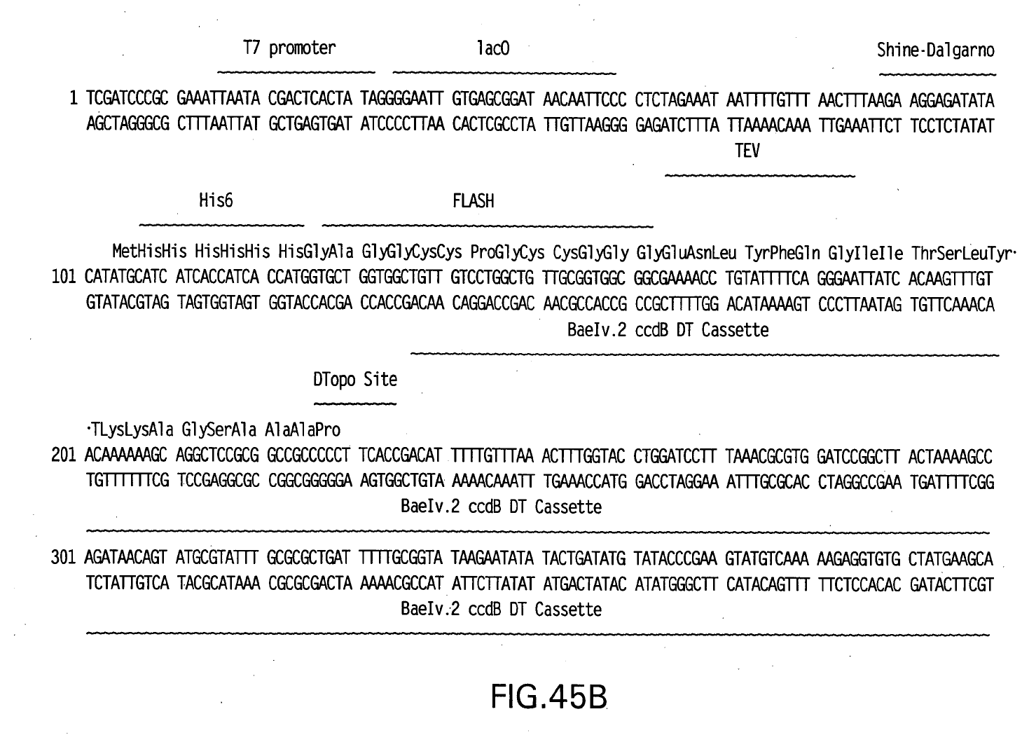

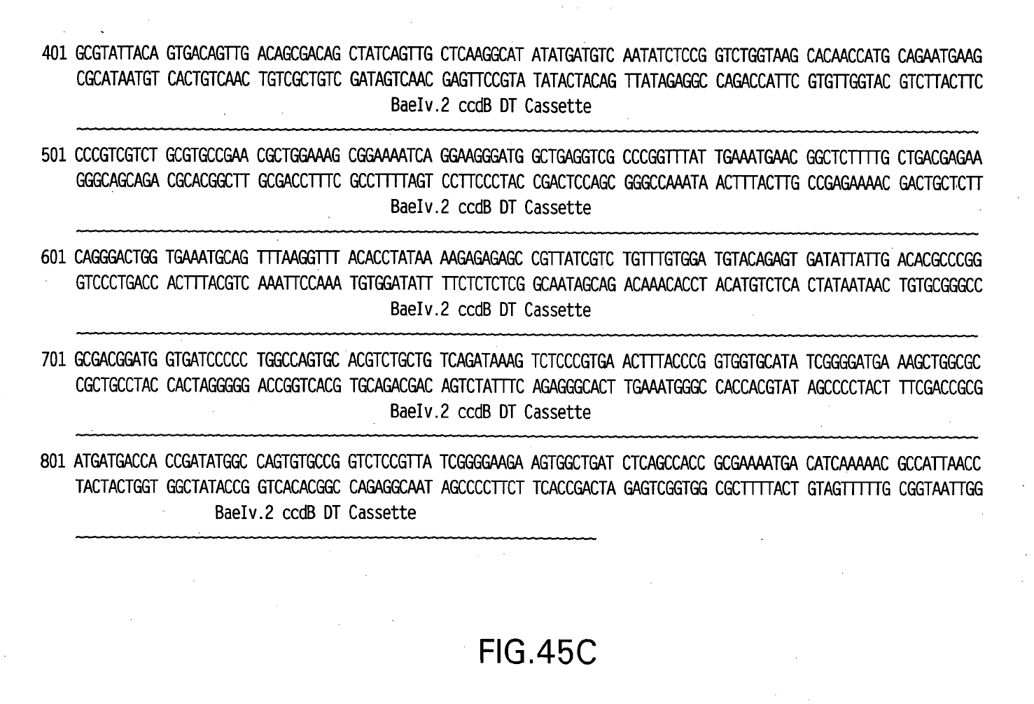

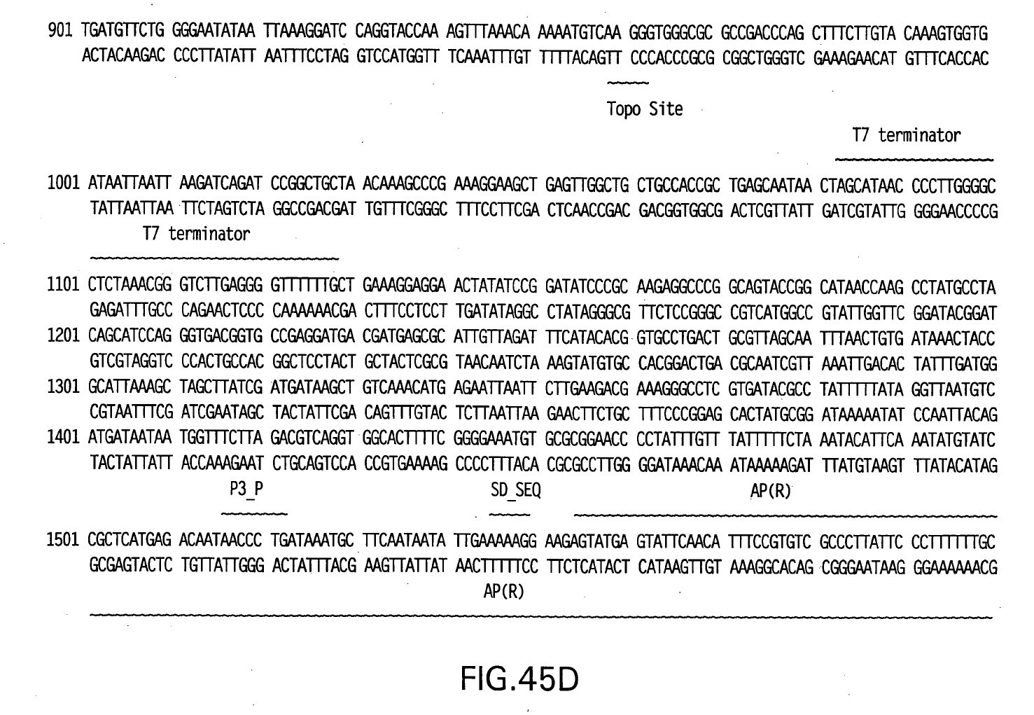

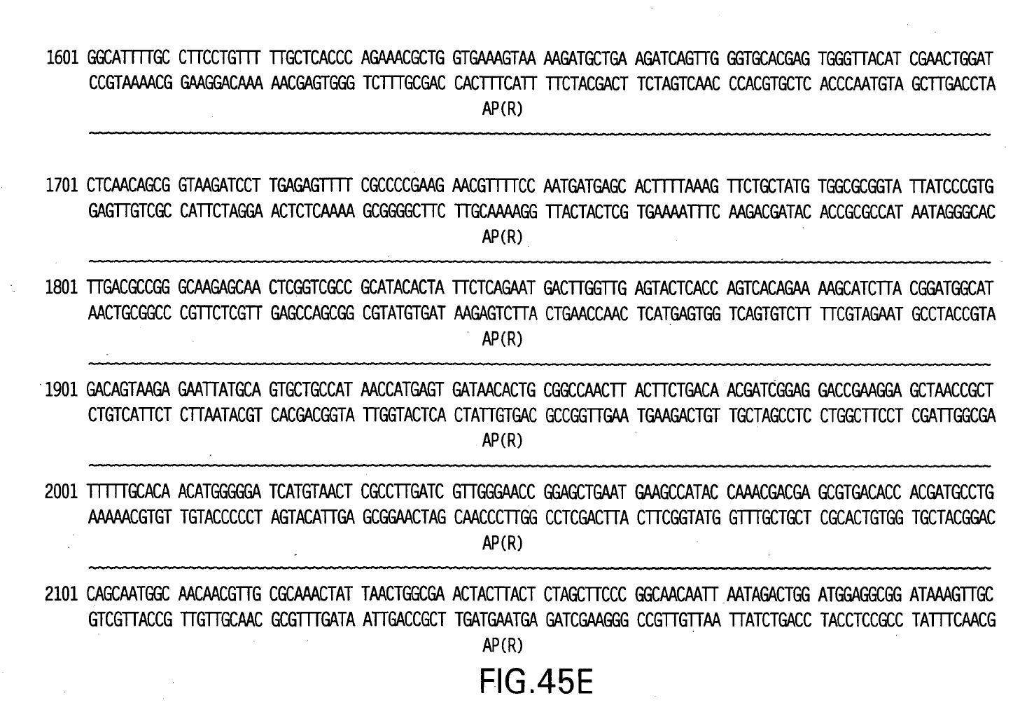

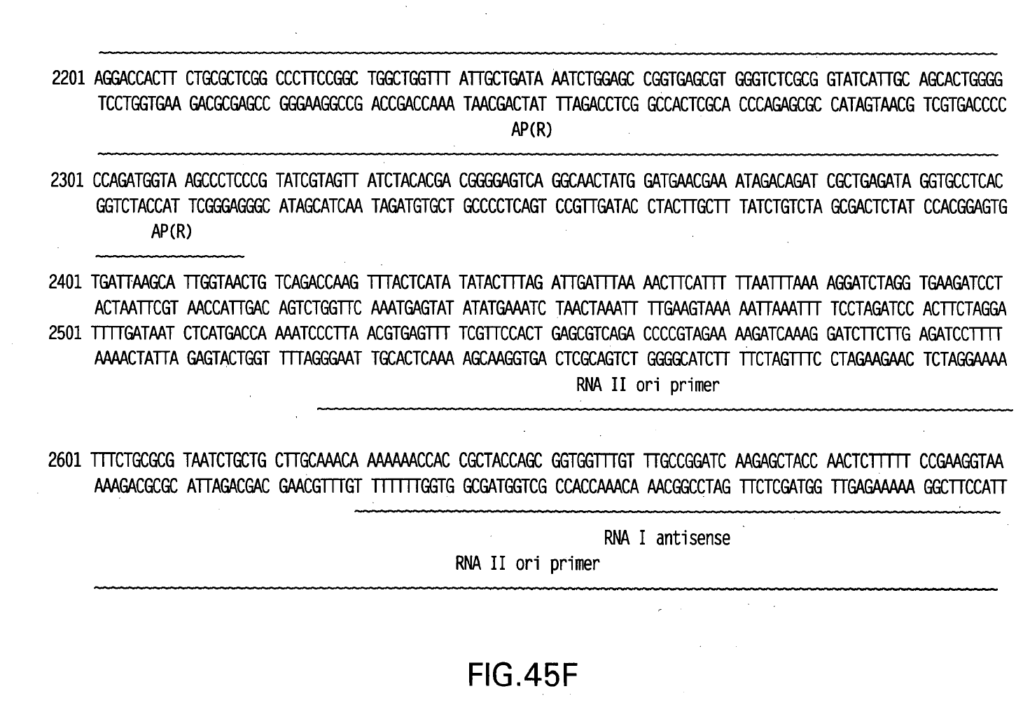

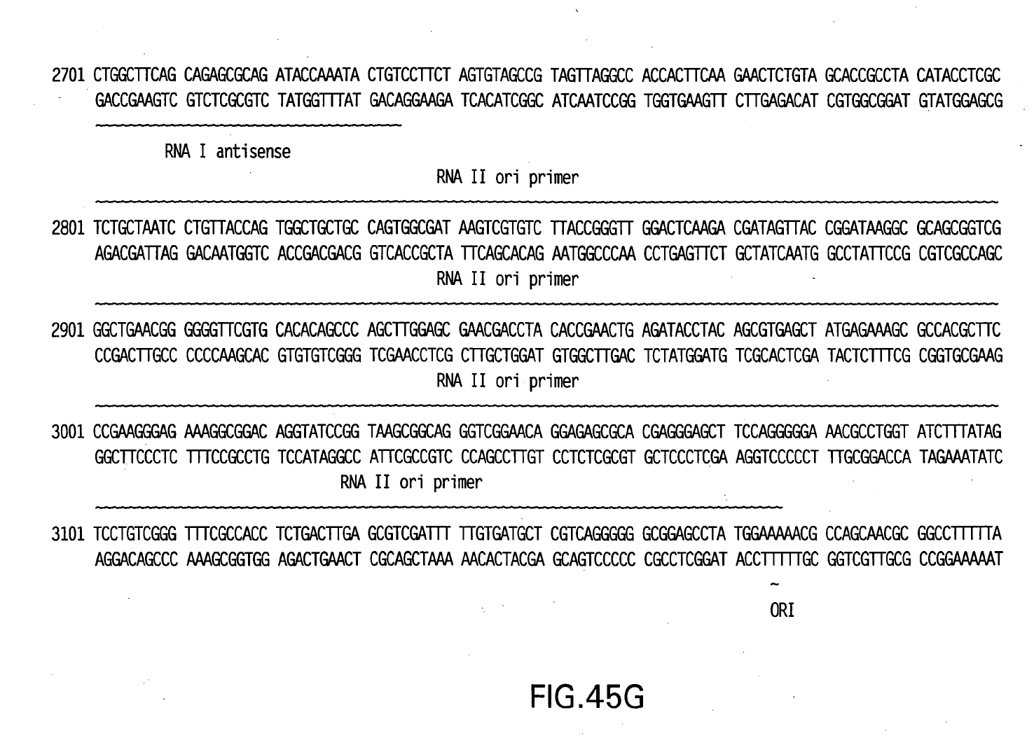

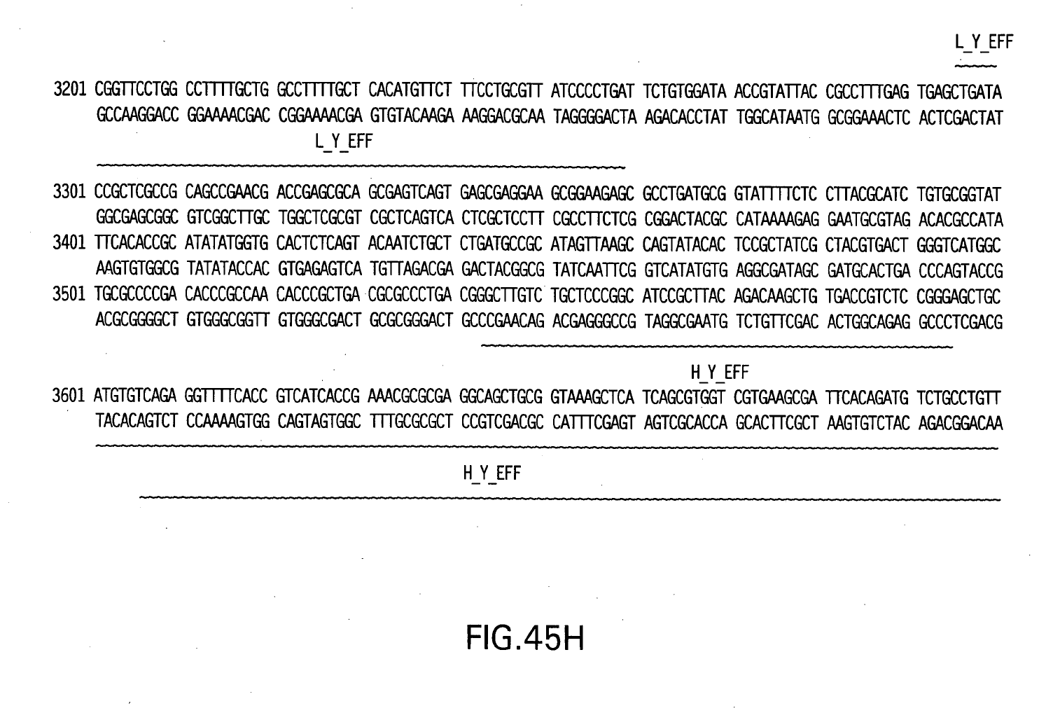

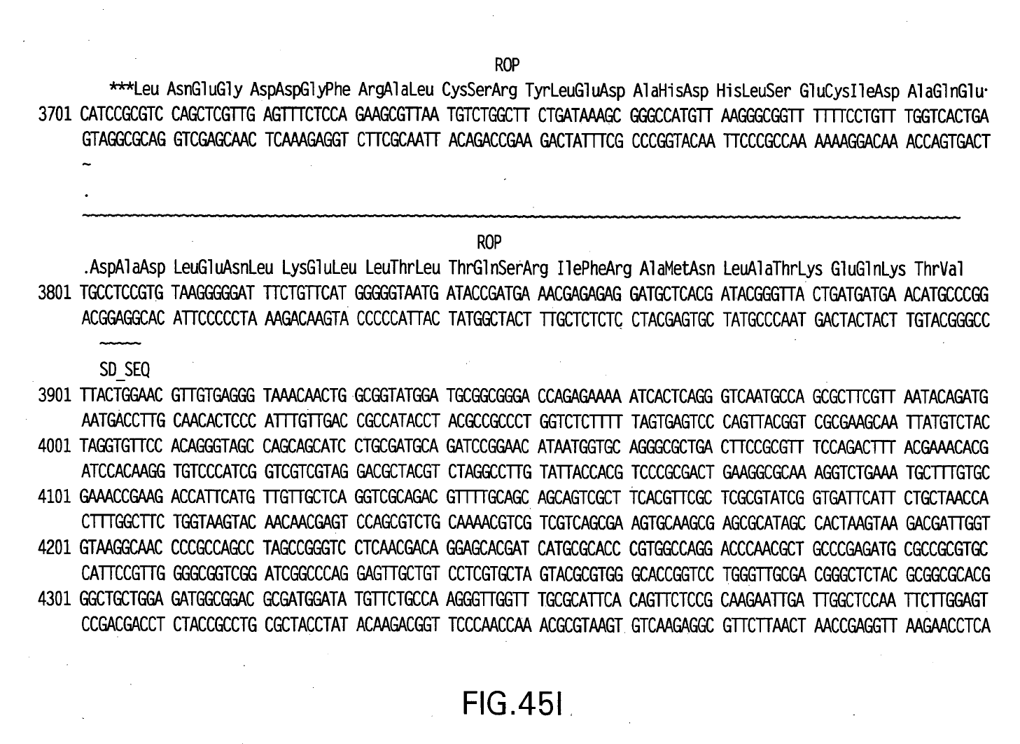

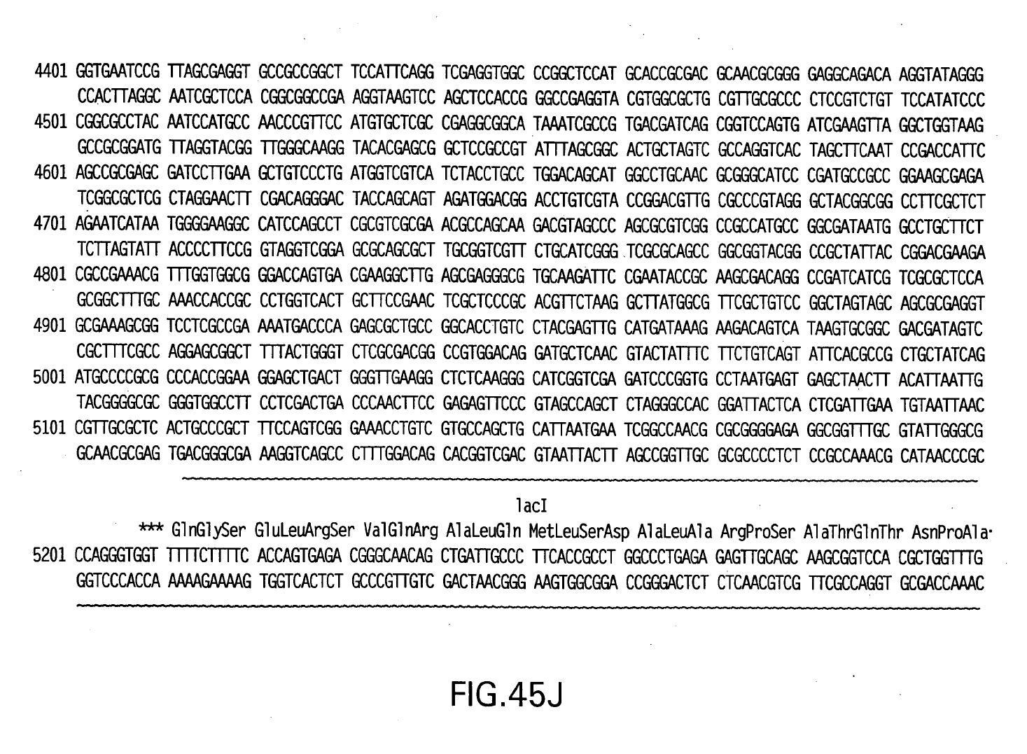

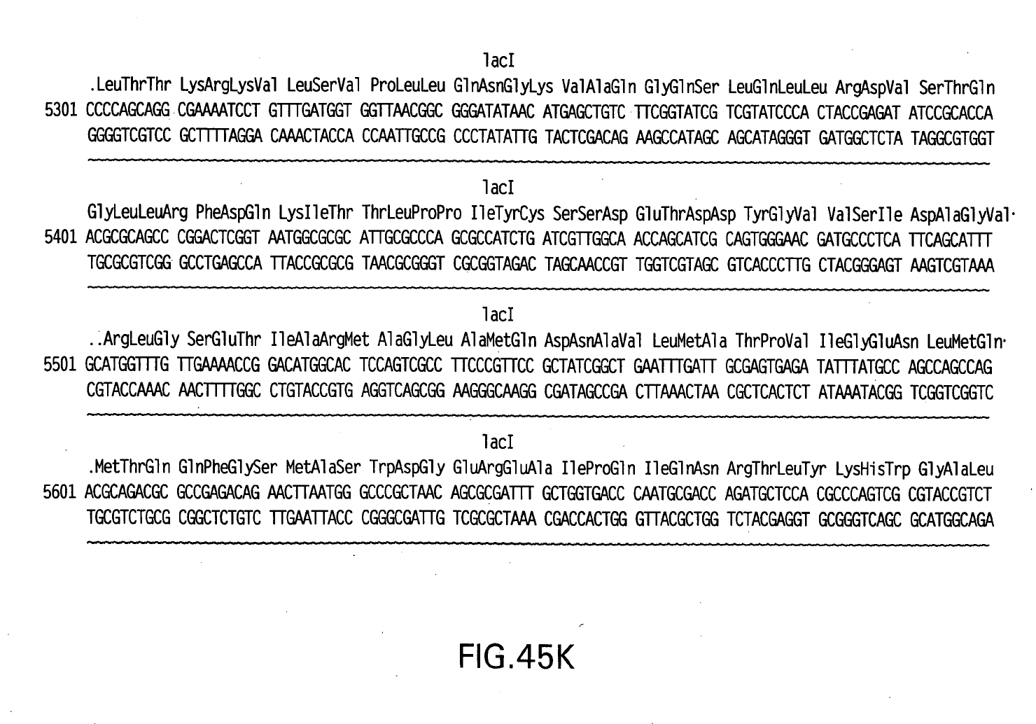

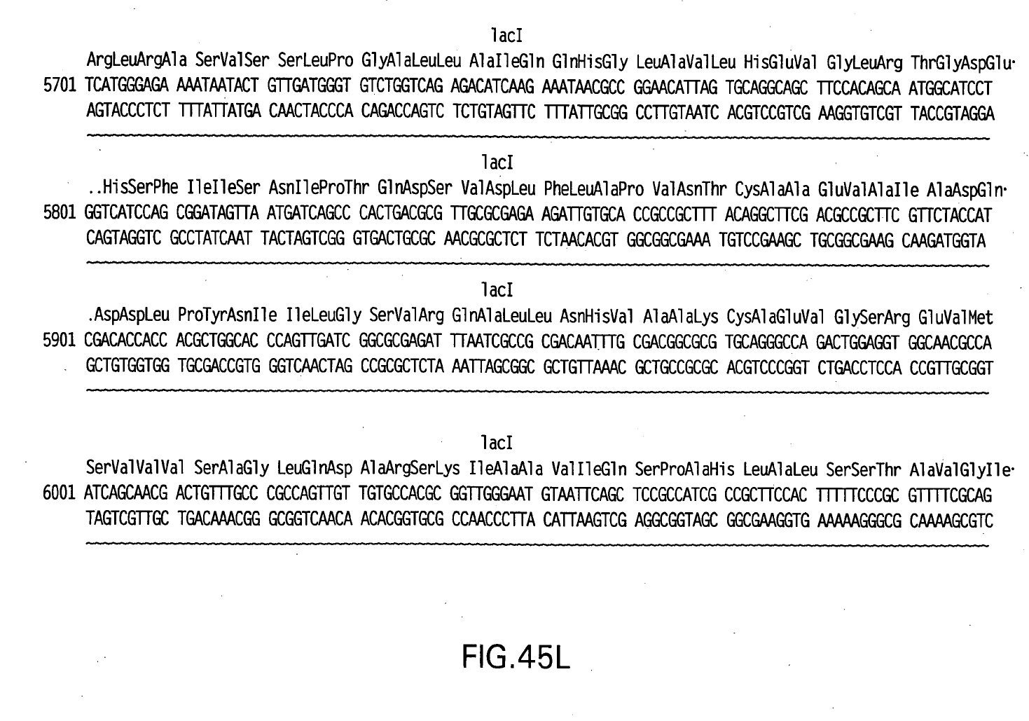

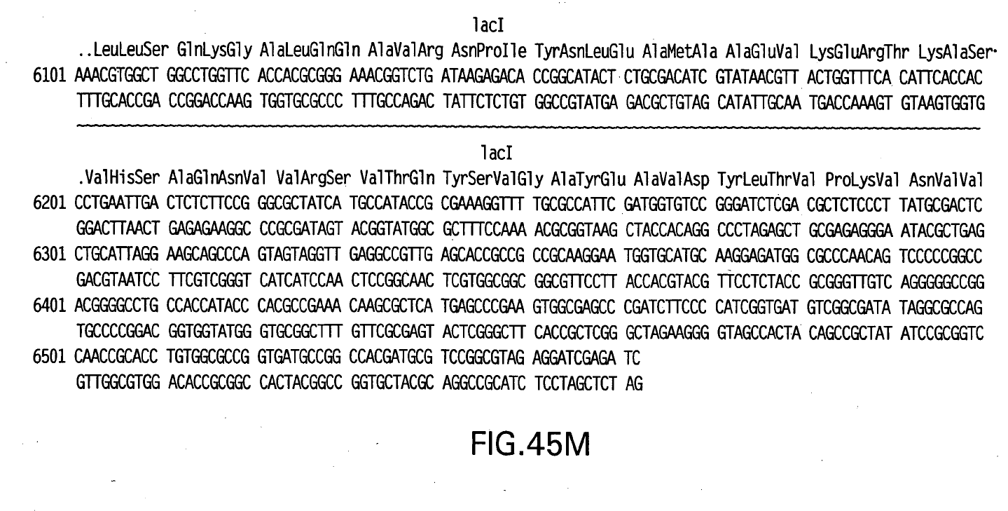

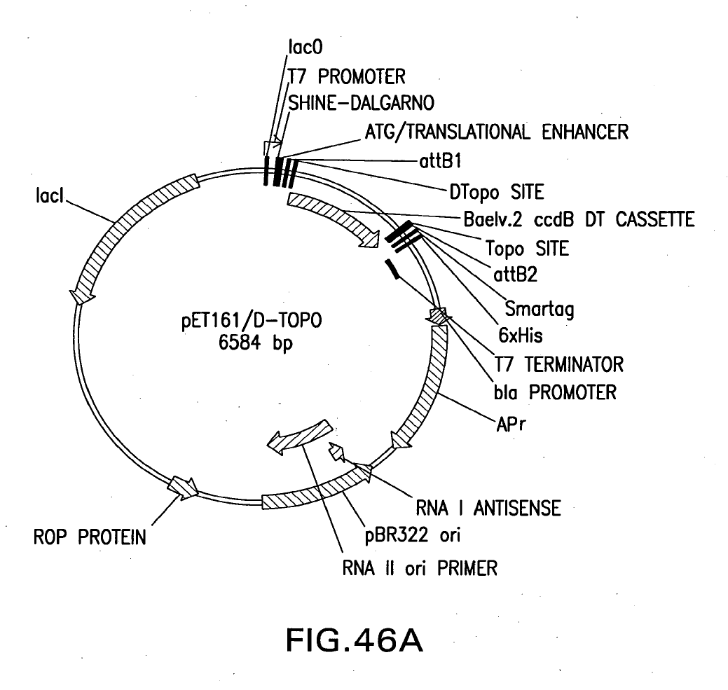

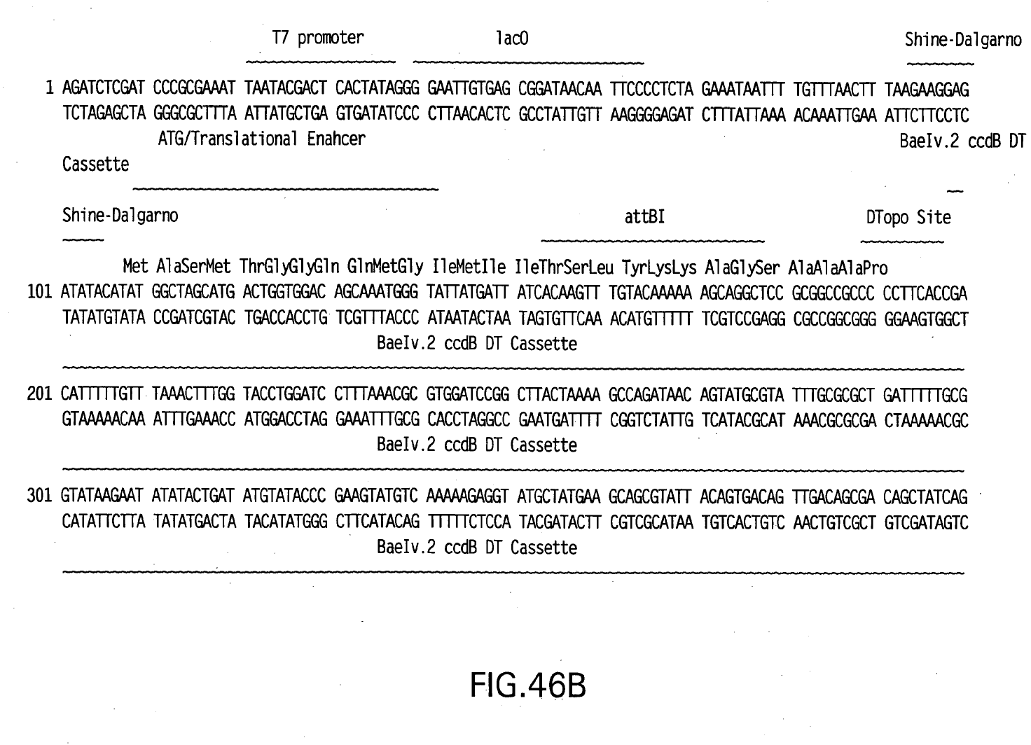

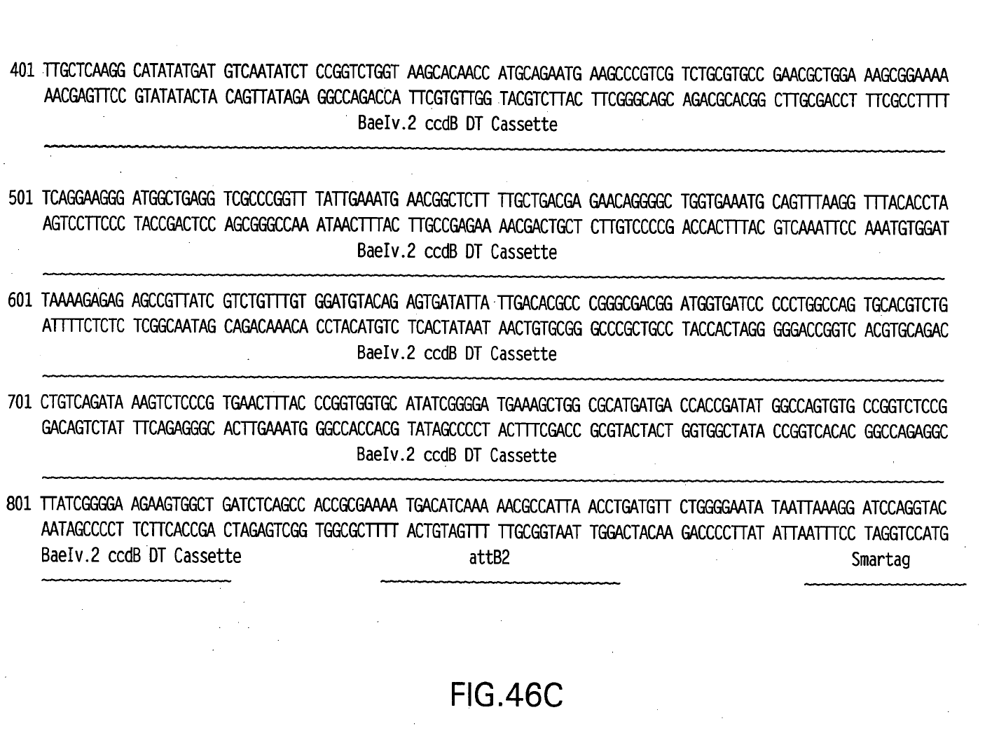

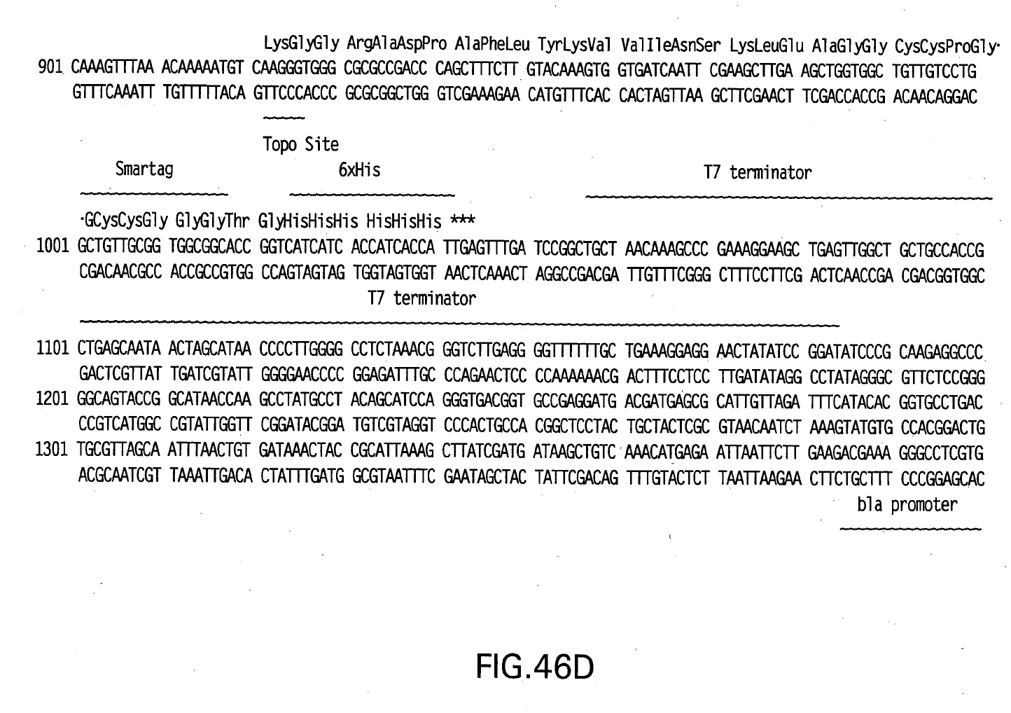

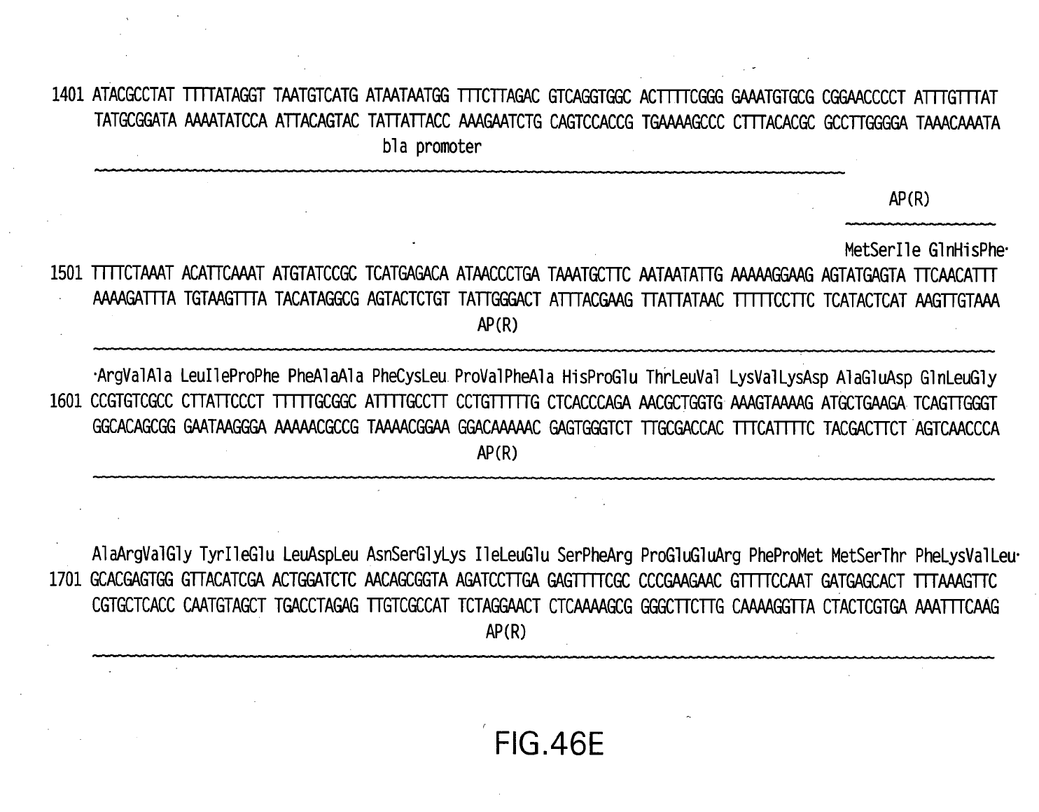

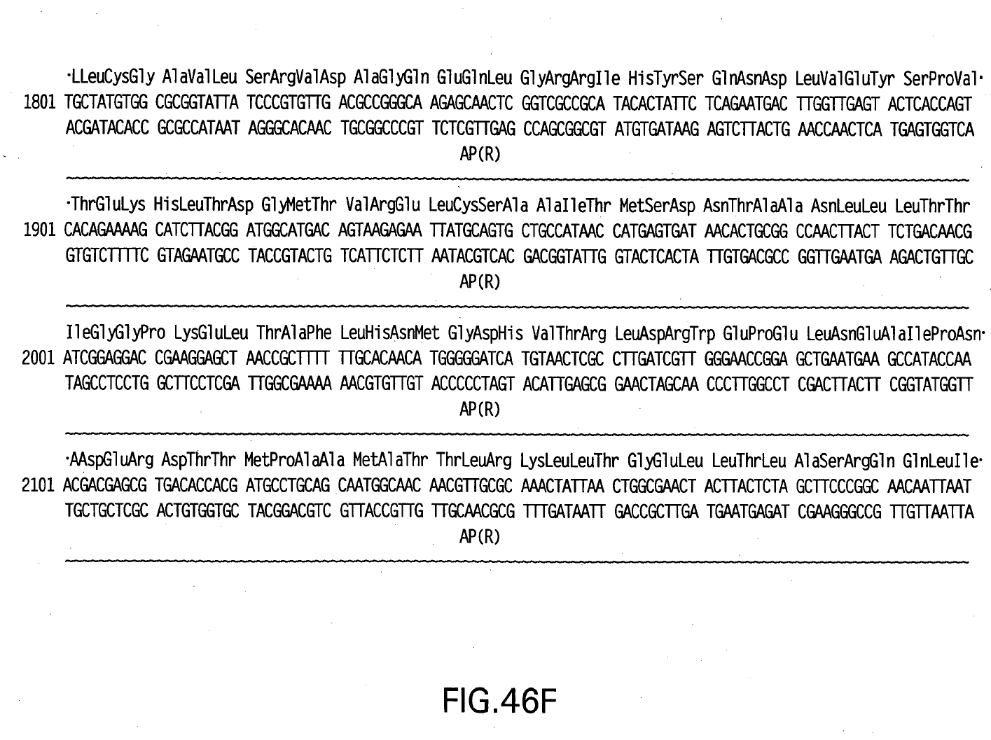

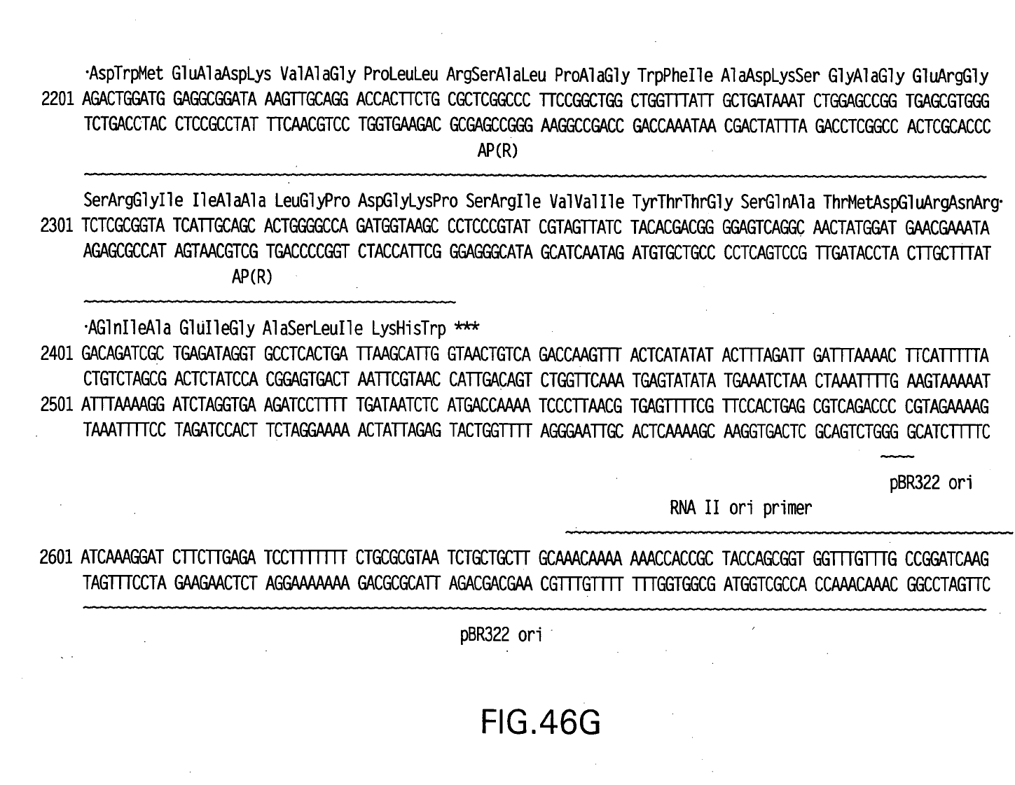

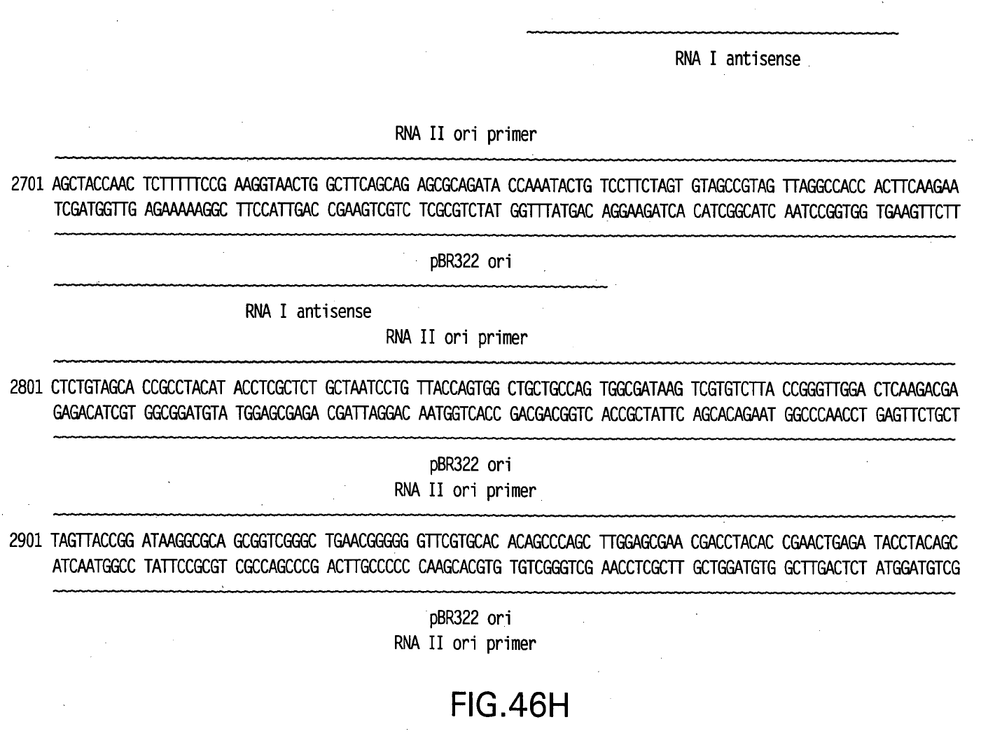

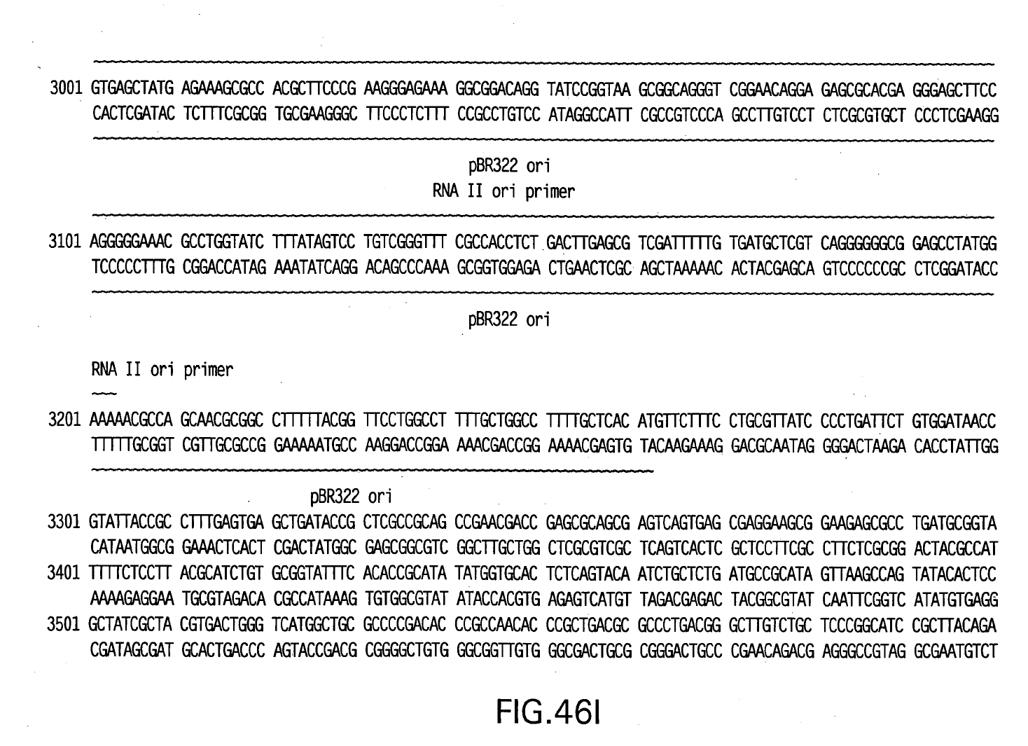

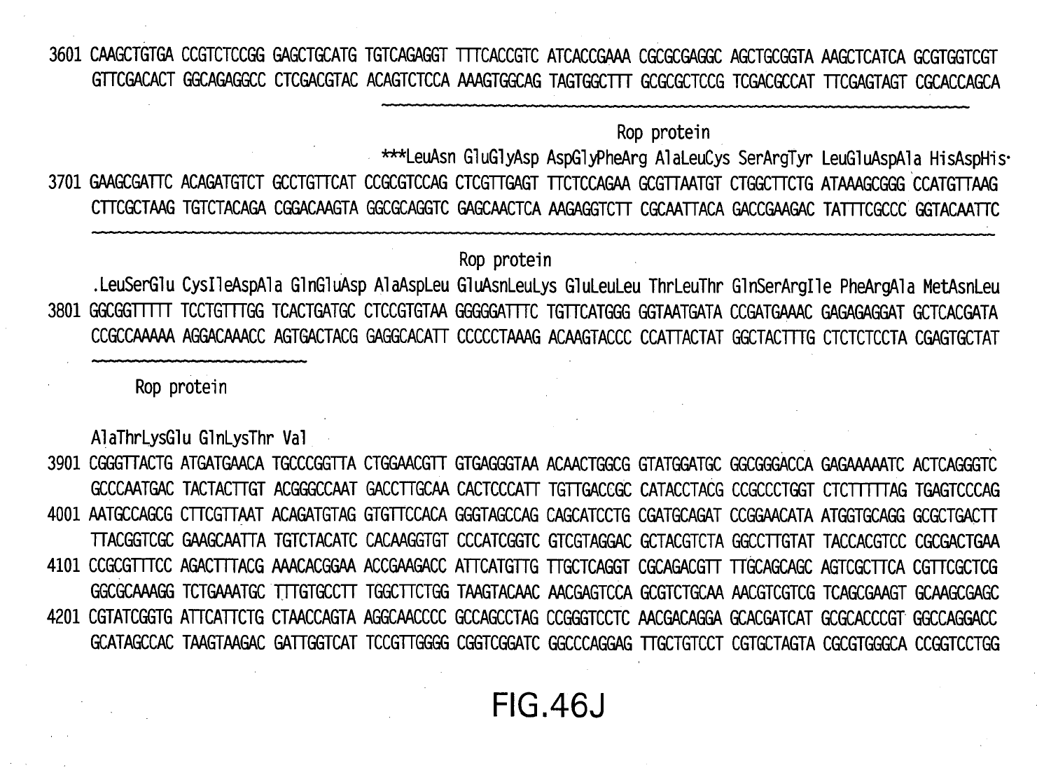

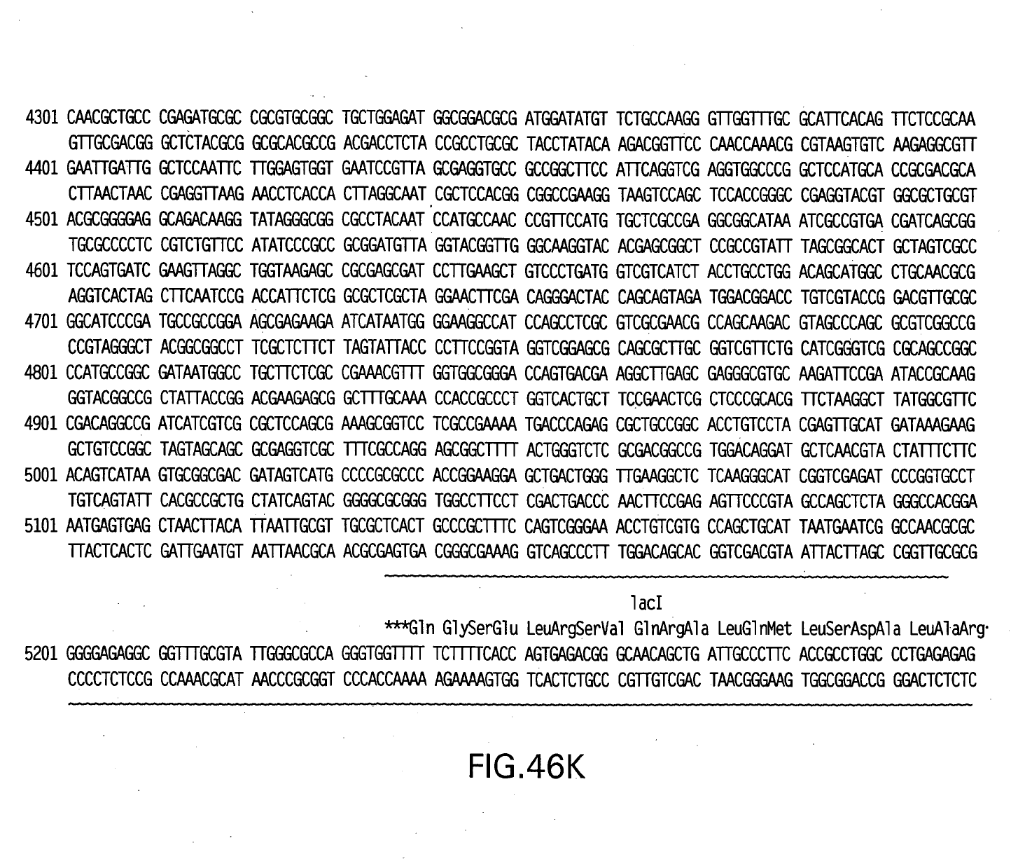

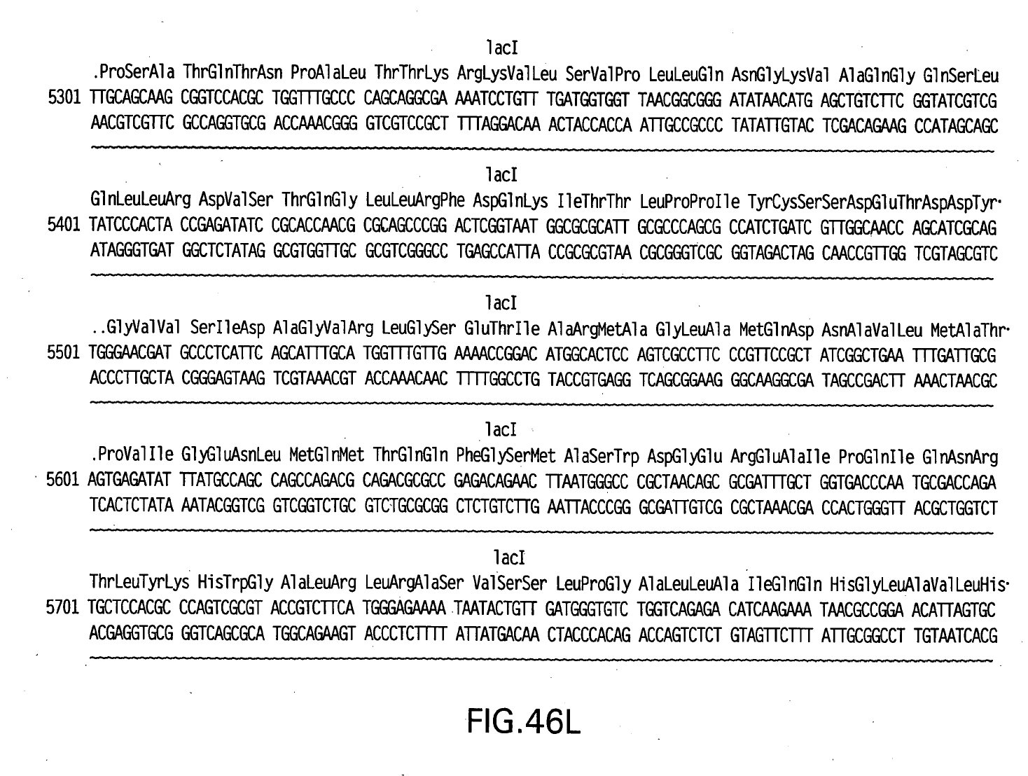

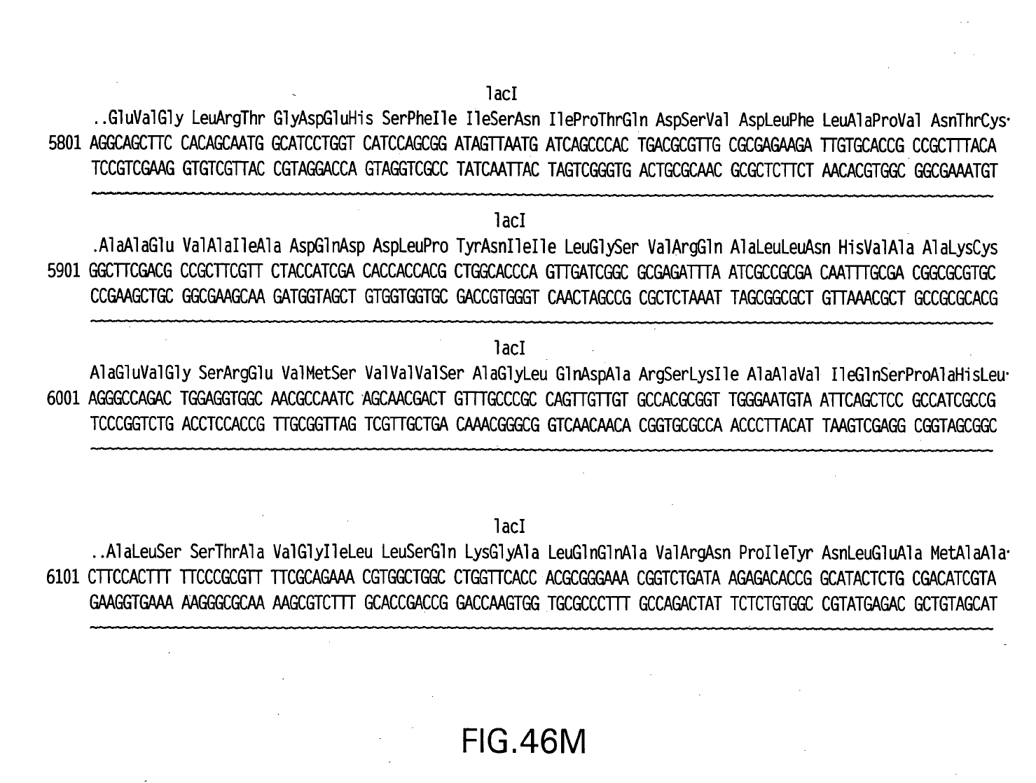

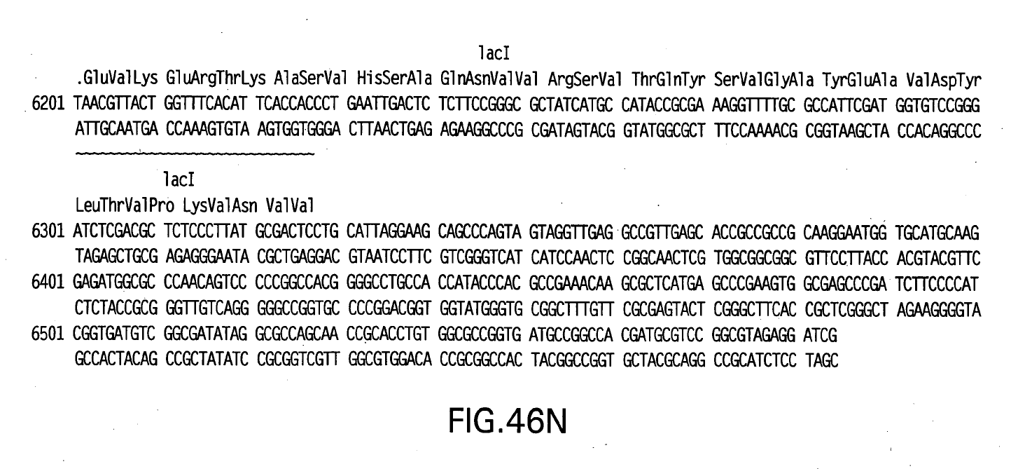

Four new pET based vectors were constructed that carry the His₆ purification and LUMIO™ detection tags on either the N-terminus or C-terminus (see FIGS. 43-46). Sequence data for these vectors is shown in figures. The N-terminal fusion vectors allow removal of tags from recombinant proteins by cleavage with TEV protease. The C-terminus vectors are engineered so that the His₆ purification and LUMIO™ detection tags are on the C-terminus of the fusion protein. The C-terminal vectors also provide a ribosome binding site, start codon and translational leader sequence to allow ORFs without these elements to be expressed. These vectors were designed as both GATEWAY® Destination and Directional TOPO™ cloning vectors. Using in-gel detection with the FLASH™ substrate, protein expression from these vectors is easily monitored. The functionality of the His₆ tags was verified by purifying expressed fusion proteins with a nickel-chelating resin and detection with either the His G or His C antibody and western blot analysis. A protein expressed as an N-terminal fusion was cleaved with TEV protease and released the epitope tags. The construction and the use of the pET-LUMIO™ vectors are described below.

Introduction

Expression of recombinant proteins plays an important role in analyzing gene regulation, structure and function. As more genome sequence data becomes available for an ever-growing list of organisms, a major challenge will be to rapidly clone and express multiple genes across several expression platforms. Additionally, sufficient quantities of proteins will be required for meaningful analysis. Finally, a rapid detection method for determining recombinant protein expression would be highly desirable. By combining the high-yield bacterial pET expression system, the state of the art GATEWAY® and TOPO™ cloning technologies and the novel LUMIO™ detection technology, researchers will be able to rapidly and efficiently clone their gene of interest, obtain high protein expression levels and detect protein expression.

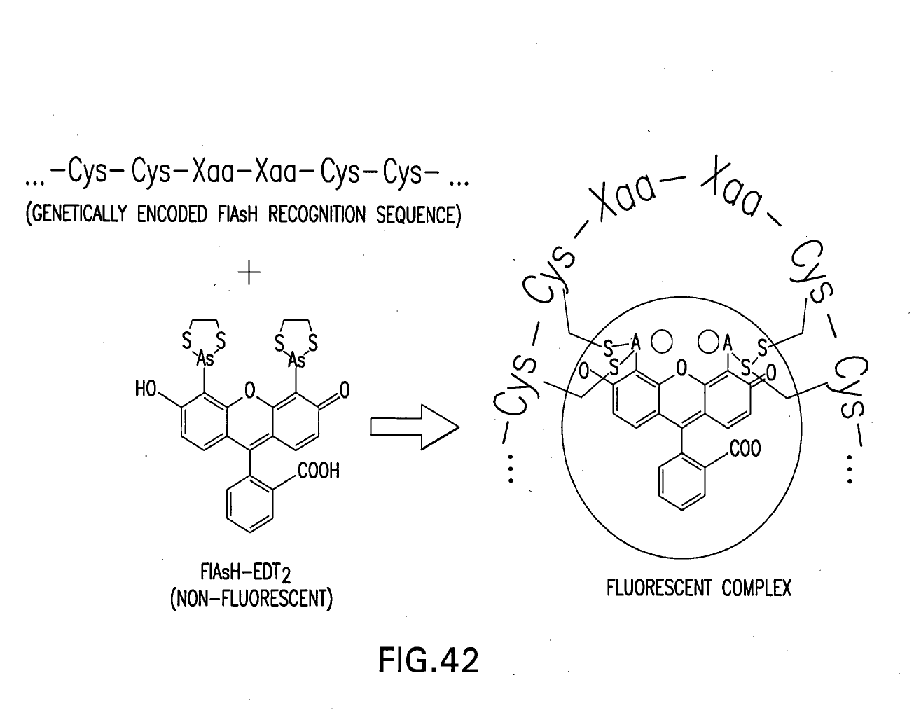

The LUMIO™ technology represents a novel fluorescent detection method that is based on the binding of a bis-arsenical fluoresceine molecule to a rare six amino acid tetracysteine motif. This technology generates rapid visual and potentially quantitative results for the detection of expressed proteins. Traditionally the attachment of fluorescent labels to proteins has required post-translational chemical modification. Alternatively, green fluorescent protein (GFP) can be filsed to the protein of interest to produce fluorescent molecules, however the size of the protein (238 amino acids) can limit its uses. By utilizing the Fluorescein Arsenical Hairpin labeling reagent (FLASH™; FIG. 42), one can label proteins that contain a tetracysteine motif of the sequence CCXXCC, where C=cysteine and X=an amino acid other than cysteine (Adams, S. R., Campbell, R. E., Groww, L. A., Martin, B. R., Walkup, G. K., Tao, Y., Llopis, J., Tsein, R. Y. J. (2002) Am. Chem. Soc., 124: 6063-6076). The FLASH™ substrate is relatively non-fluorescent until it covalently binds to the target motif when it becomes strongly fluorescent. The optimal fluorescence is excited at 528 nm but is also excited and visible on a standard UV box using an EtBr filter (Griffin, A. B., Adams, S. R., Tsein, R. Y. (1998) Science 281:269-272). The FLASH™ substrate can be incorporated into standard Lammeli loading dye (Lammeli, V.K. 1970. Theor. Appl. Genet. 85:882-888) and added to protein samples prior to heat denaturation. After SDS/PAGE separation, the FLASH™ stained recombinant proteins can be visualized on a UV light-box.

The sequence Cys-Cys-Pro-Gly-Cys-Cys was shown to give the complex much more stability and increased affinity (Adams, S. R., Campbell, R. E., Groww, L. A., Martin, B. R., Walkup, G. K., Tao, Y., Llopis, J., Tsein, R. Y. J. (2002),Am. Chem. Soc., 124: 6063-6076). The sequence Ala-Gly-Gly was added to the N-terminus and Gly-Gly-Gly to the C-terminus to avoid any interference with the surrounding motifs and to better present the epitope when generating antibodies against the tag. The DNA sequence encoding the LUMIO™ and spacer regions was also altered to avoid hairpin loops, palindromes, dimer formation and the use of any rare tRNA codons. This twelve amino acid tag was chosen as the motif to be inserted into the pET vectors.

The optimized LUMIO™ Binding Motif is ALA GLY GLY CYS CYS PRO GLY CYS CYS GLY GLY GLY (SEQ ID NO:131). An exemplary nucleotide sequence which encodes this sequence is GCT GGT GGC TGT TGT CCT GGC TGT TGC GGT GGC GGC (SEQ ID NO:132).

Four new pET based Destination and d-TOPO™ vectors, referred to as pET160-DEST, pET161-DEST, pET160/D-TOPO™, and pET161/D-TOPO™, were constructed that contained the LUMIO™ detection motif (FIGS. 43, 44, 45, and 46). The pET-160/LUMIO™ expression vectors encode the His₆ purification signal, the LUMIO™ tag and the TEV protease cleavage site on the resulting N-terminus of the recombinant protein. The pET161/LUMIO™ expression vectors encode the His₆ purification signal and the LumIO™ tag located at the C-terminus of the resulting recombinant protein. The pET161 vectors also have a RBS, ATG and translation initiation sequence upstream of the att sites to promote strong initiation of ORFs placed between the att sites. These plasmids are derivatives of pET11b (Studier, F.W., Rosenberg, A. H., Dunn, J. J. and Dubendorff, J. W. (1990) Meth. Enzymol. 185:60-89) which have a low copy vector backbone, a T7 promoter for high level expression and the lacI/lacO control region downstream of the T7 promoter for tightly regulated gene expression. By combining the strengths of the GATEWAY® system with the high expression pET vectors and the LUMIO™ technology, these vectors provide researchers with valuable tools aimed at reducing the time and effort associated with protein discovery. The data presented herein demonstrates that this system is well suited for GATEWAY® mediated gene expression and high-throughput protein expression proteomic applications and in-gel detection of the expressed products.

Materials and Methods

Bacterial Strains and Growth Conditions.

The Escherichia coli strains TOP10, DH5alpha, and DB3.1 were used for cloning while BL21 Star [F⁻ ompT hsdS_{B} (r_{B}⁻m_{B}⁻) gal dcm rne131 (DE3)] was used for gene expression. Standard media and growth conditions were used for E. coli (growth at 37° C. in LB). Ampicillin was used at 100 μg/ml in plates and media. Chloramphenicol was used at 10 μg/ml in plates and media. Kanamycin was used at 25 μg/ml in plates and media.

Construction of pET160/LUMIO™-DEST.

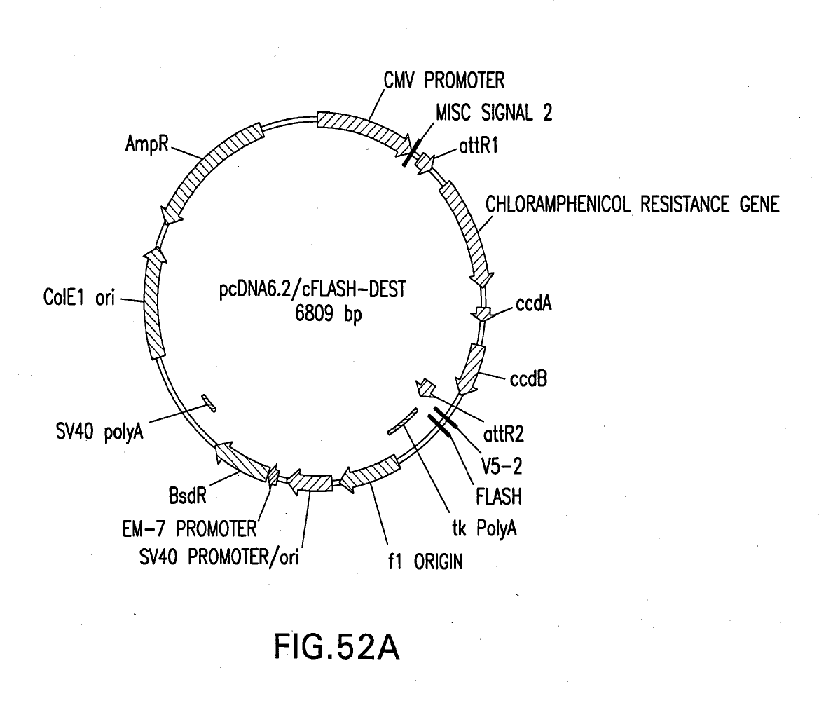

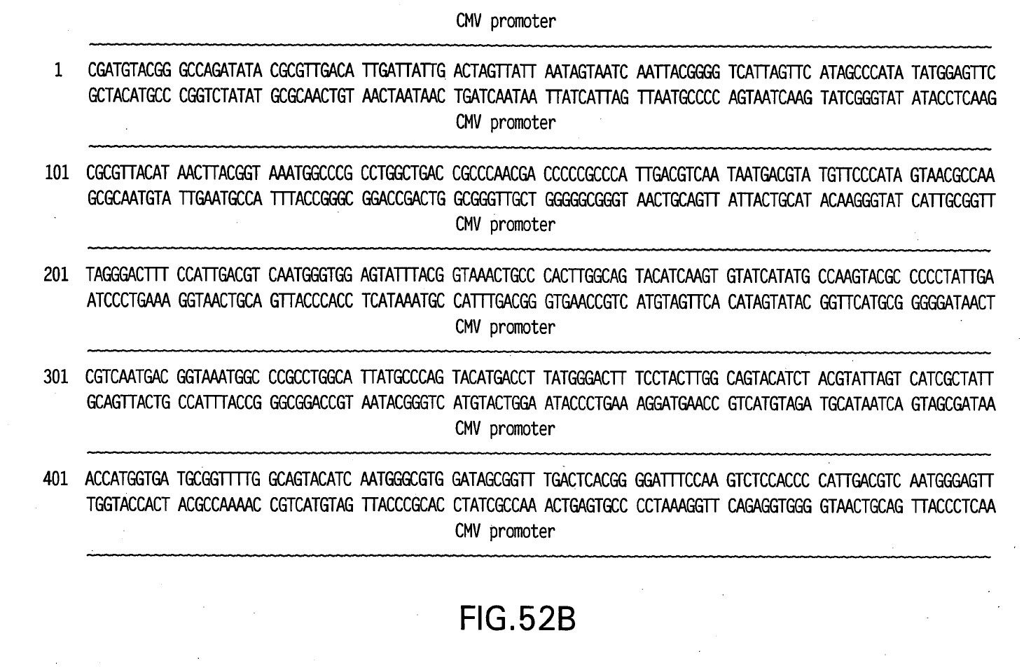

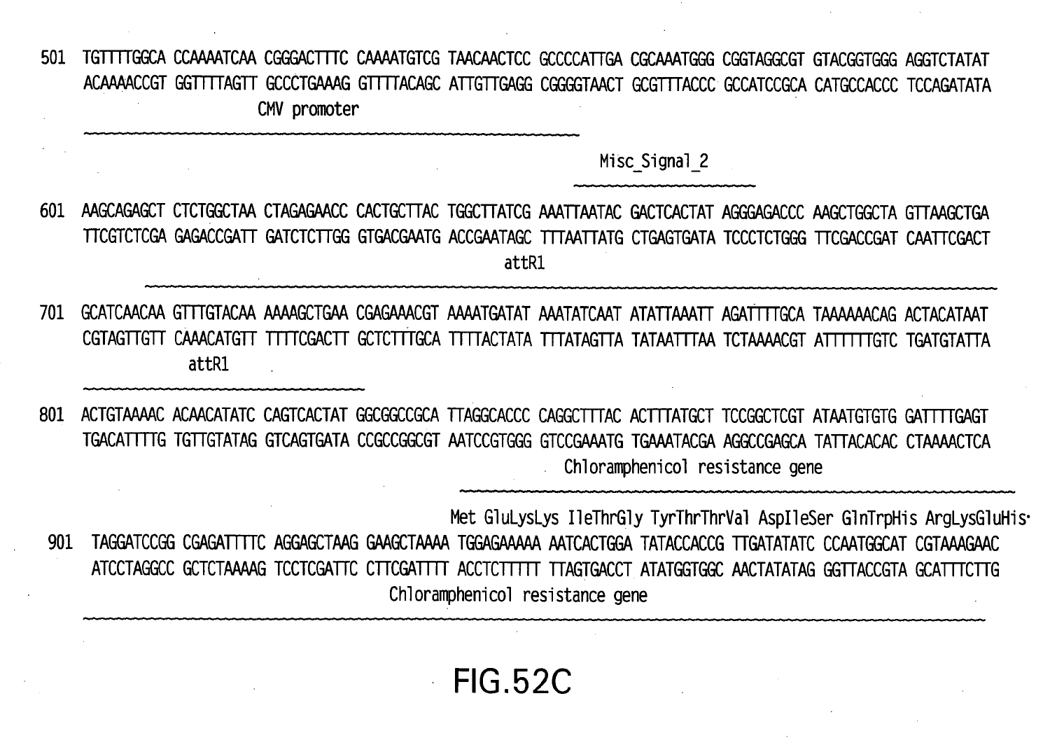

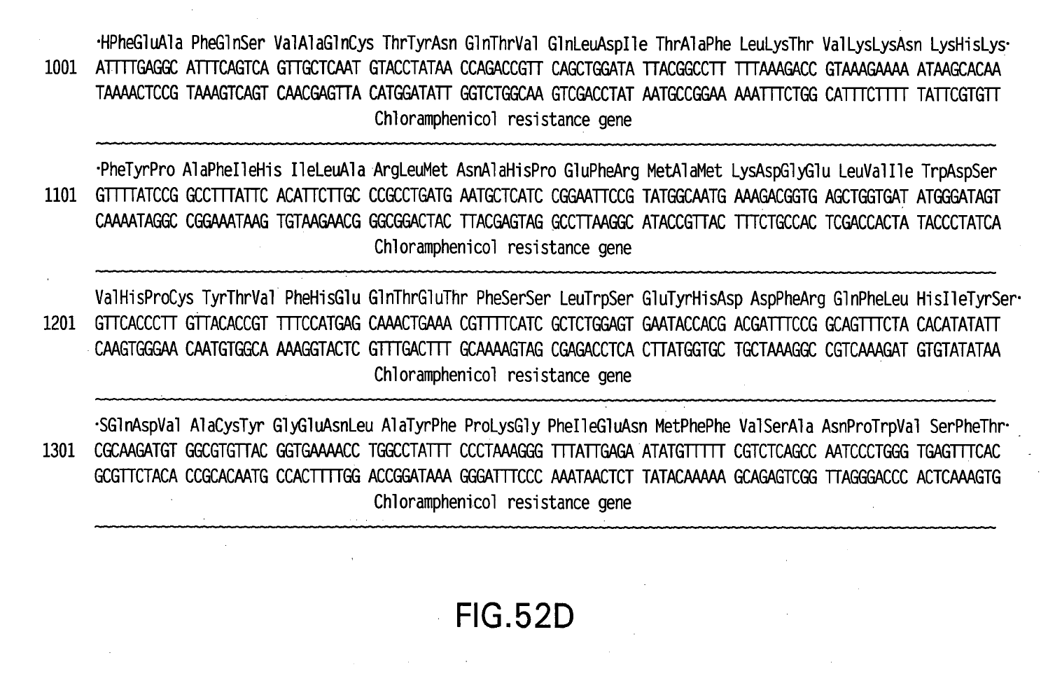

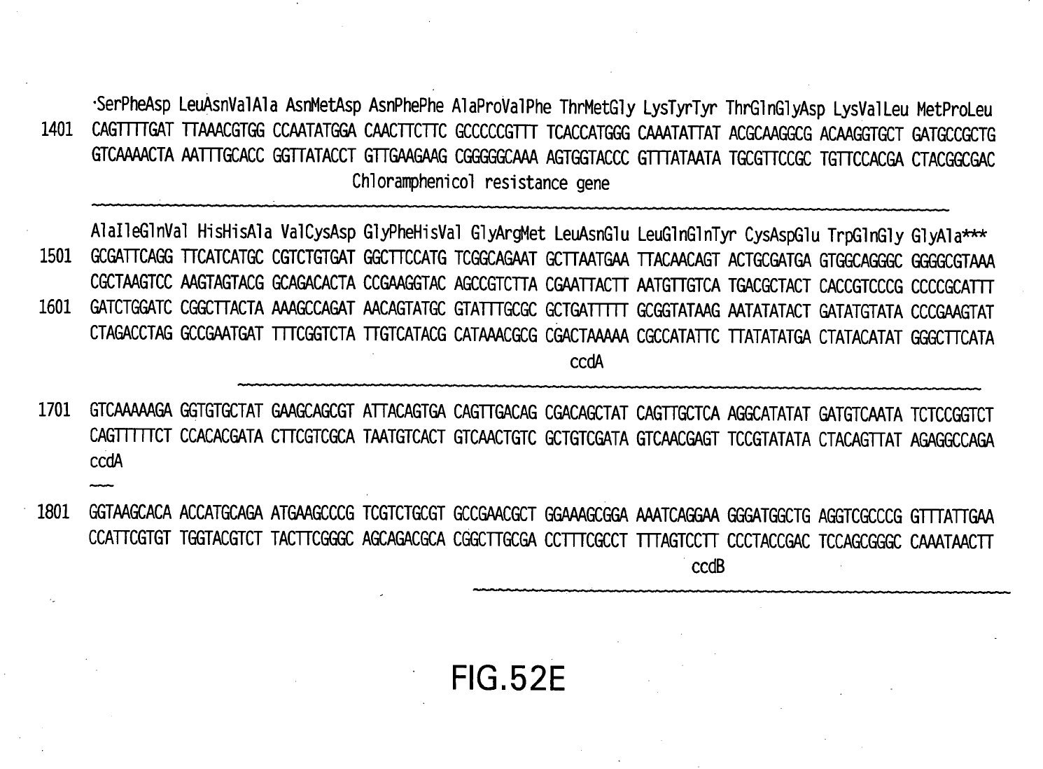

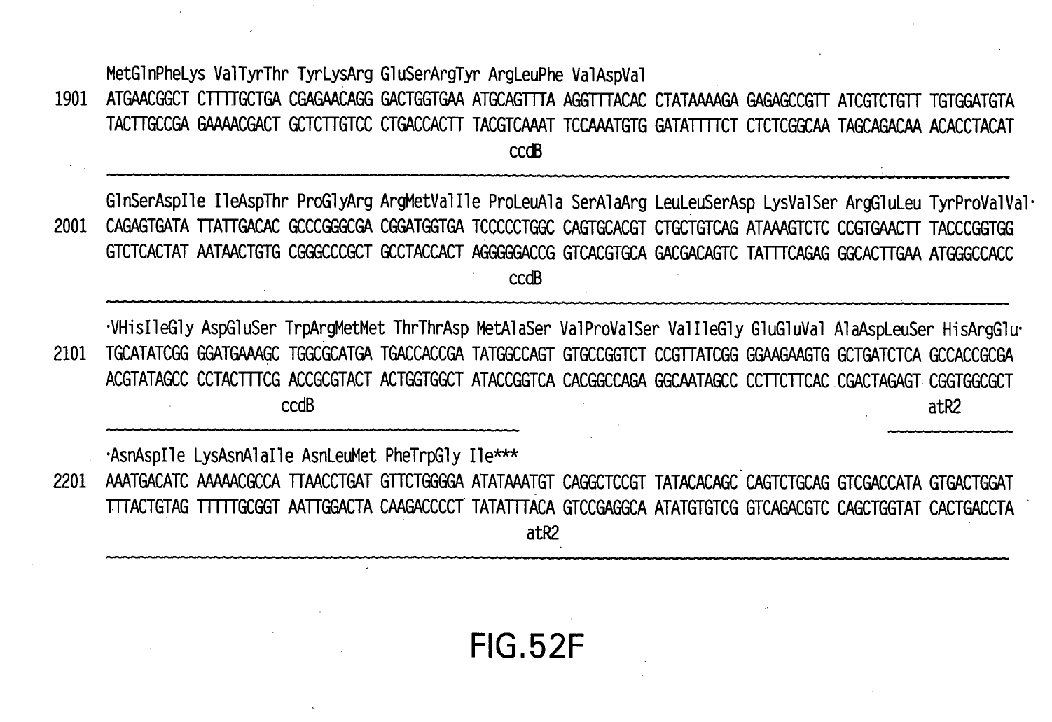

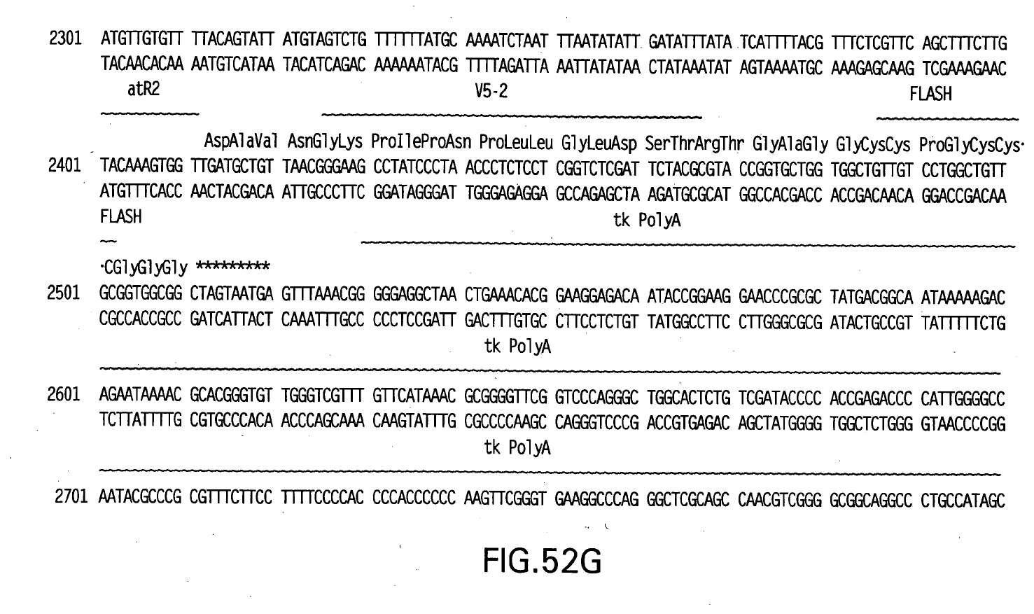

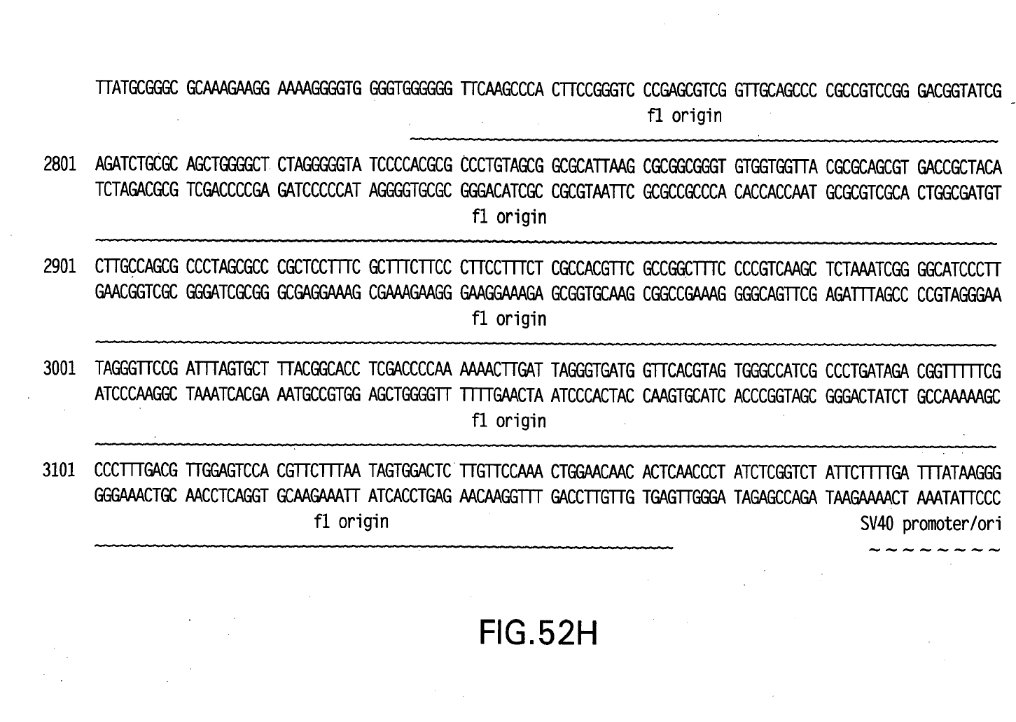

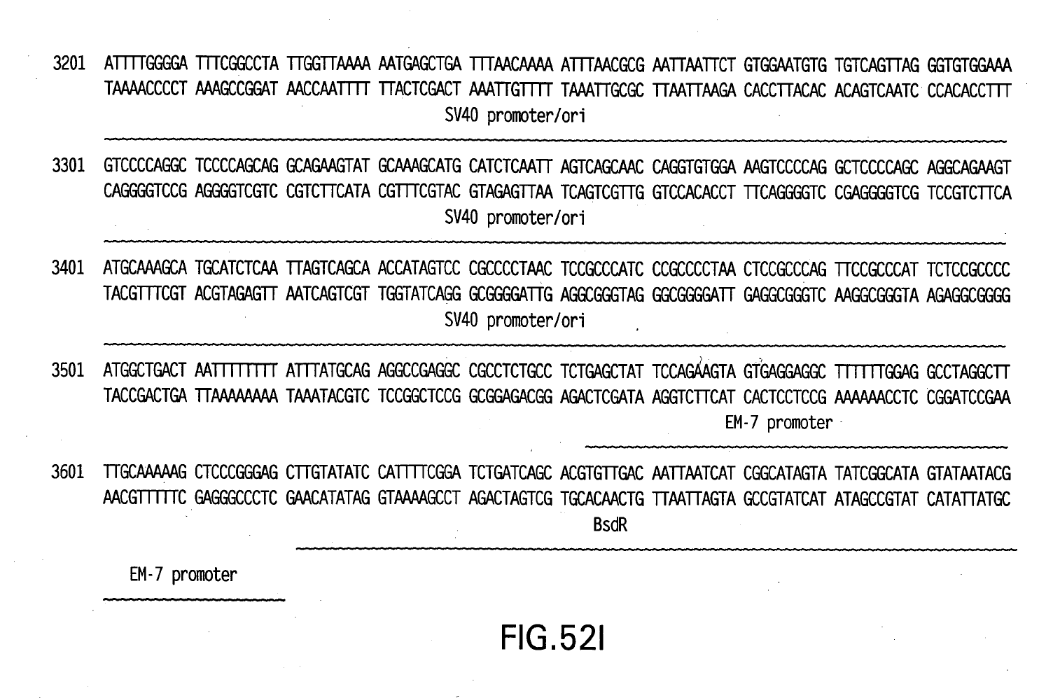

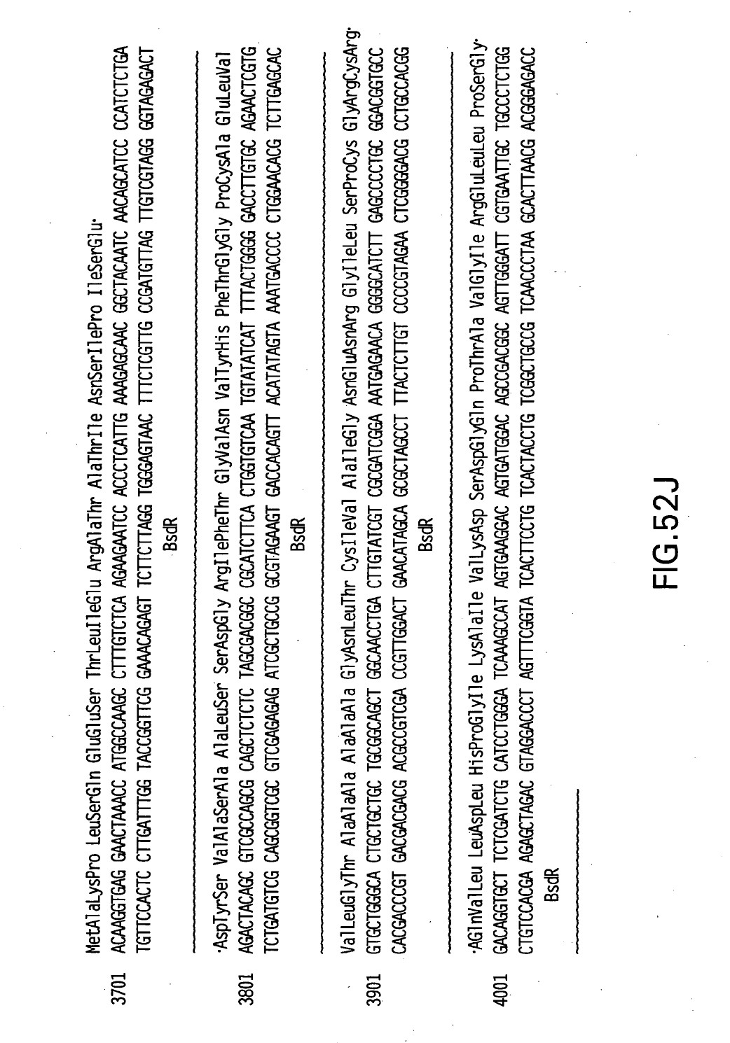

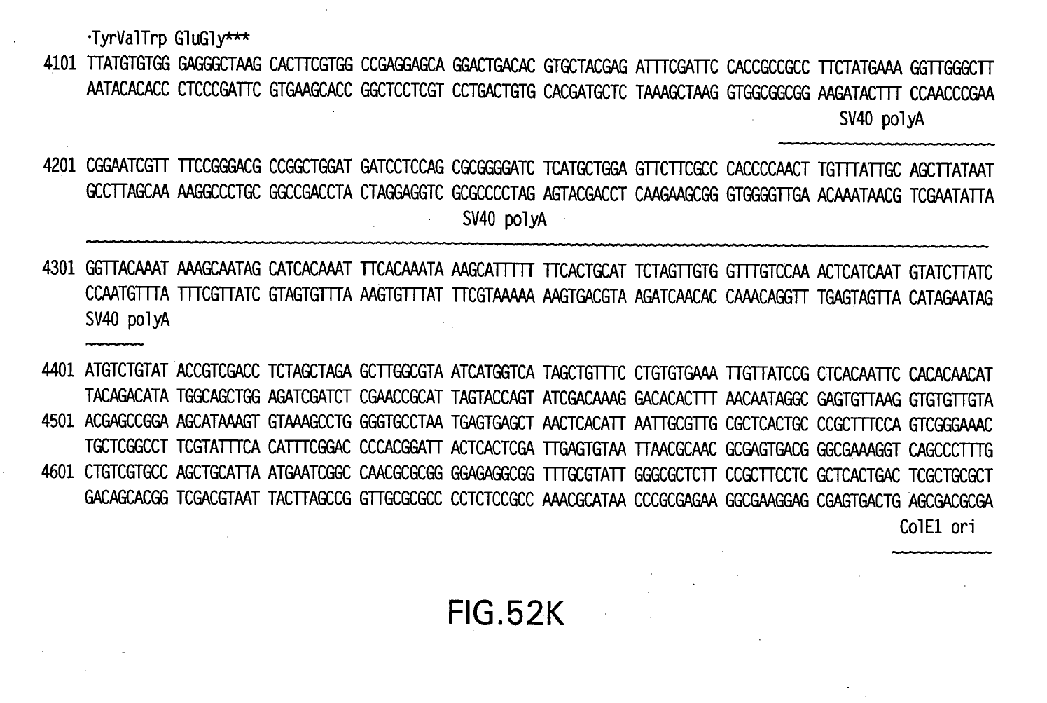

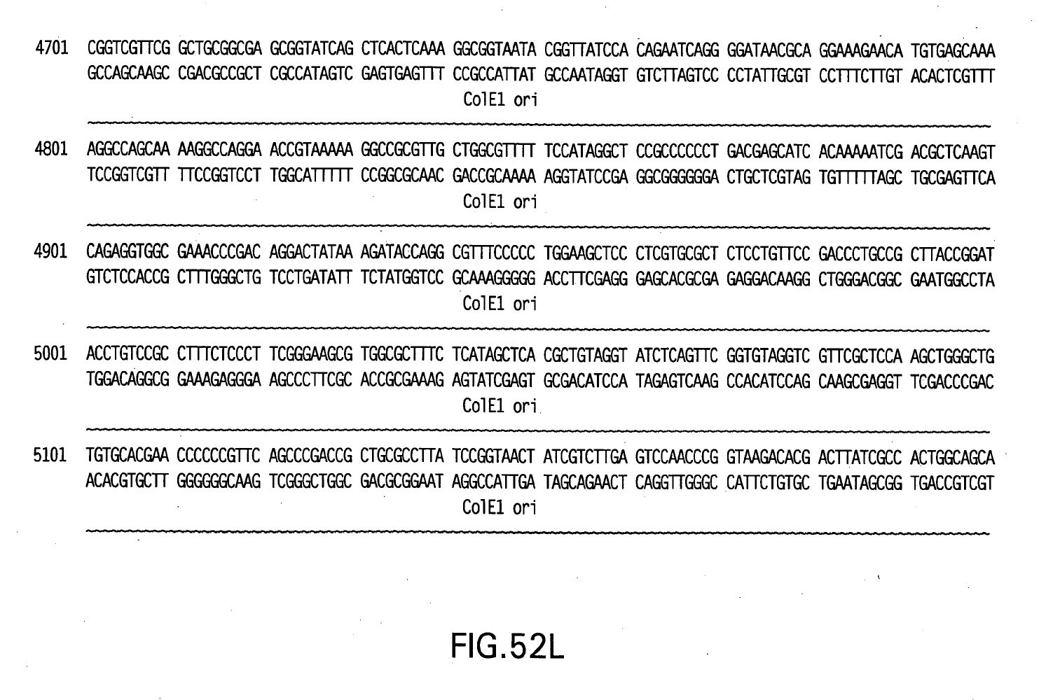

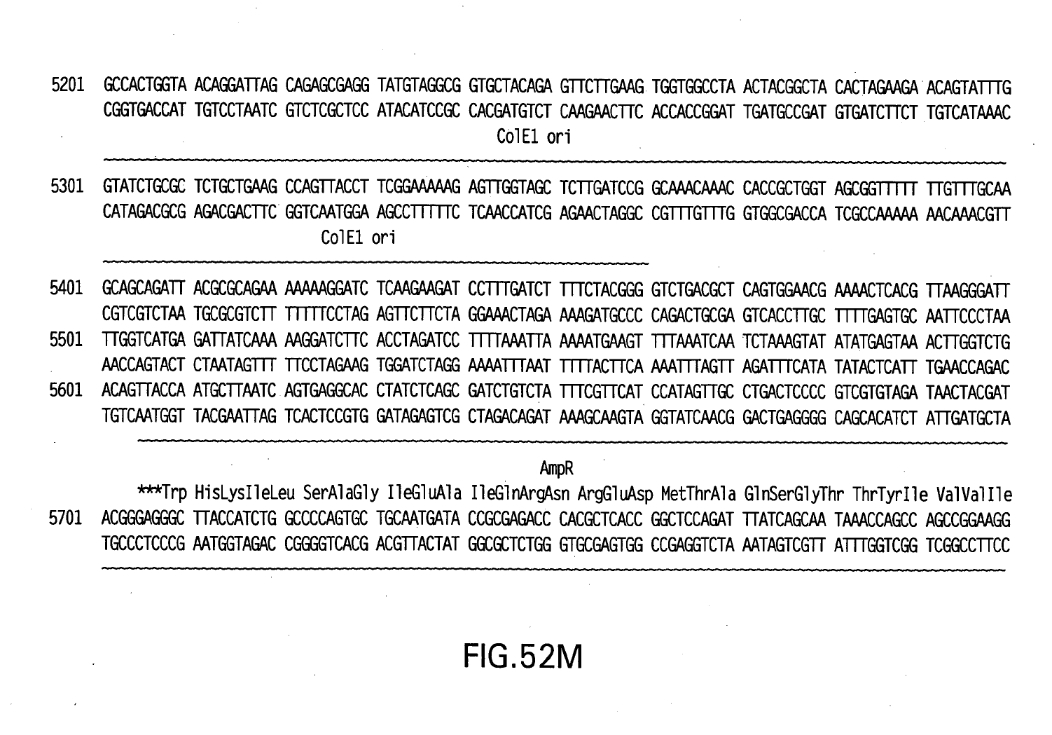

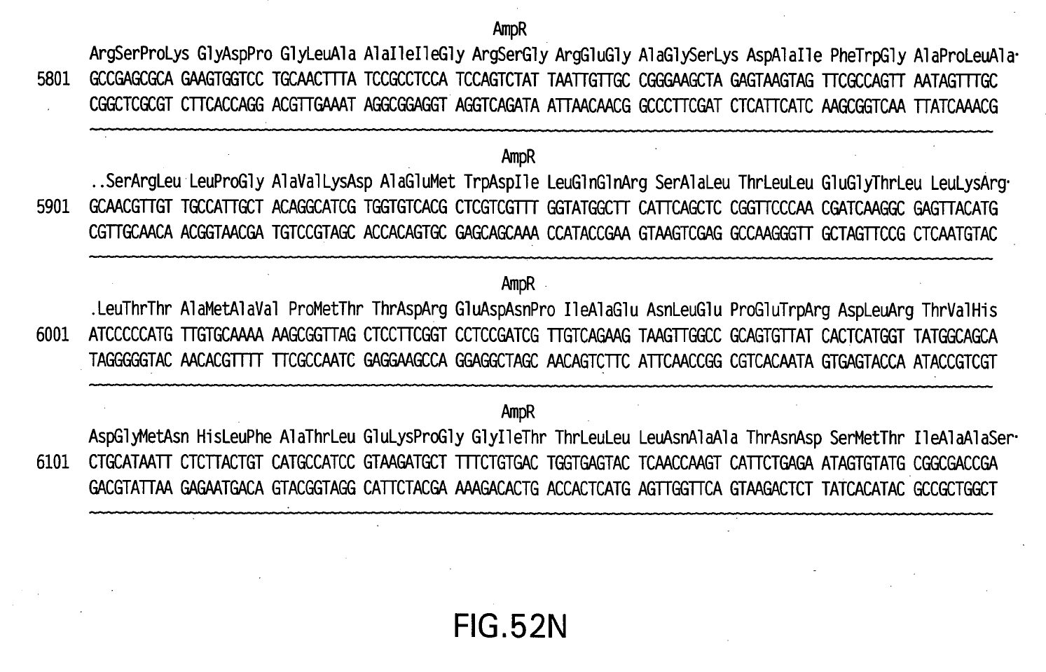

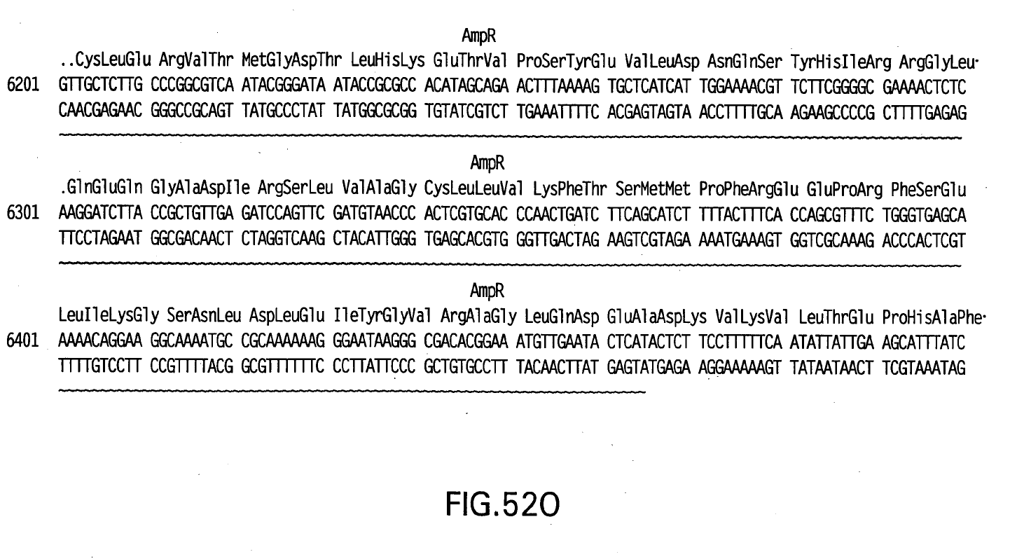

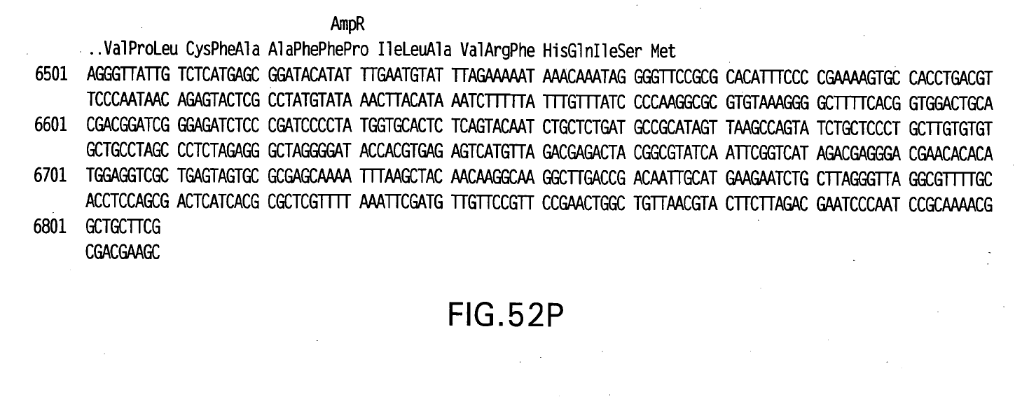

The vector was constructed by mutagenesis of pET-DEST151 (FIG. 52). A forward primer was synthesized that replaces the V5 epitope tag with the LUMIO™ tag and encoded the TEV cleavage sequence. An existing primer was used as the reverse primer to amplify the fragment. The primer sequences were as follows:

A. For151tev1:

(SEQ ID NO: 133)

B. 5′ CCATGGTGCTGGTGGCTGTTGTCCTGGCTGTTGC
C. GGTGGCGGCGAAACCCTGTATATTCAGGGAATTATC 3′
#448 PCR.BLUNTT137:

(SEQ ID NO:134)

5′AGACTTTATCTGACAGCAGACGTG 3′

The two primers were mixed together with the pET-DEST151 template and the fragment was amplified by PCR. The PCR product was cloned into pCR2.1 (Invitrogen Corp., Carlsbad, Calif., cat no. K2000-01). After sequence verification, the pCR2.1/FLASH™ TEV vector was restriction digested with NcoI, gel purified, and inserted into the NcoI digested and SAP treated pET-DEST151 backbone by standard methods. Ligations were transformed into DB3.1 cells (Invitrogen Corp., Carlsbad, Calif., cat no. 11782-018) and cells were plated on LB plates containing chloramphenicol. Positive clones were sequence verified.

Construction of pET161/LUMIO™-DEST.

The vector was constructed by cassette mutagenesis of pET-DEST42 (Invitrogen Corp., Carlsbad, Calif., cat no. 12276-010). Two primers were synthesized that when annealed created an oligo containing the LUMIO™ and His₆ sequence flanked by BstBI and AgeI sites. The two primers (5′42TOP, 5′42BOT) were used together in a PCR reaction.

5′42TOP

(SEQ ID NO:135)

5′ CGAAGCTTGAAGCTGGTGGCTGTTGTCCTGGCTGTTGCGGTG
GCGGCACCGGTCATCATCACCATCACCATGGTTGA 3′
5′42BOT

(SEQ ID NO:136)

5′CCGGTCAACCATGGTGATGGTGATGATGACCGGTGCCGCCACCGC
AACAGCCAGGACAACAGCCACCAGCTTCAAGCTT 3′

The reaction was placed in a thermocycler and run through 5 cycles of 96° C. for two minutes, 55° C. for 1 minute, and 72° C. for 1 minute. The product was cloned into pCR2.1. A positive clone was sequence verified and digested with BstBI and AgeI. The purified fragment was ligated into BstBI and AgeI digested pET-DEST42 to create the pET-DEST42/FLASH™ vector. Ligations were transformed into DB3.1 cells, and cells were plated on LB plates containing chloramphenicol.

The pET-DEST42/FLASH™ (also referred to as pET-DEST42F) was further developed by the addition of the N-terminal RBS, ATG and translational enhancer sequences. First, the pET-DEST42/FLASH™ and pET-DEST151 were digested with BglII and NotI and the N-terminal fragment of pET-DEST151 was purified and ligated into the pET-DEST42/FLASH™ backbone to provide convenient cloning sites upstream of the att sites. The new vector, pET-DEST42/151/FLASH™ clone was digested with NdeI and NotI and ligated to a similarly digested PCR product generated from pET-DEST42 using the For NdeI 42 and Rev NdeI 42 primers. This PCR fragment provides the NdeI/ATG upstream of the attRI site allowing the vector to accept the translational enhancer. Colonies were screened by digestion with NcoI and a positive clone sequence verified.

| | |
|---|---|
| D. For Nde 42 | |
| 5′ CGGAGGTCATATGATTATCACAAGTTTG 3′ | (SEQ ID NO:137) |
| E. Rev NdeI 42 | |
| 5′ GAAAATCTCGCCGGATCCTAACTC 3′ | (SEQ ID NO:138) |

The translational enhancer was added by digesting the pET-DEST42/151/FLASH™/NdeI plasmid with XbaI and NdeI. A PCR fragment containing the first eleven amino acids of the T7 gene 10 protein followed by an Asel site was generated by PCR using the pET-24 (Novagen, 441 Charmany Drive, Madison, Wis., 53719, cat. no.69772-1) vector as a template. The PCR fragment was digested with XbaI and AseI and ligated into the vector backbone and ligations were transformed into DB3.1 cells, and cells were plated on LB plates containing chloramphenicol. A positive clone was sequence verified from the T7 promoter through the attR1 site.

#6 T7 Forward

(SEQ ID NO:139)

5′TAATACGACTCACTATAGGGG 3′
F. T7 Tag Rev

(SEQ ID NO: 140)

5′ CGGTCGGATTAATAGCGATTTGCTGTCC 3′

TOPO™ Adaptation of pET160/LUMIO™-DEST and pET161/LUMIO™-DEST.

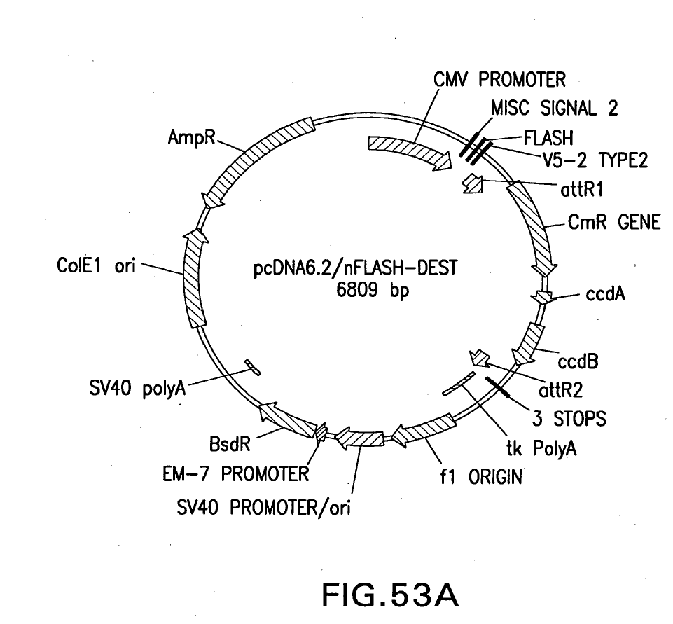

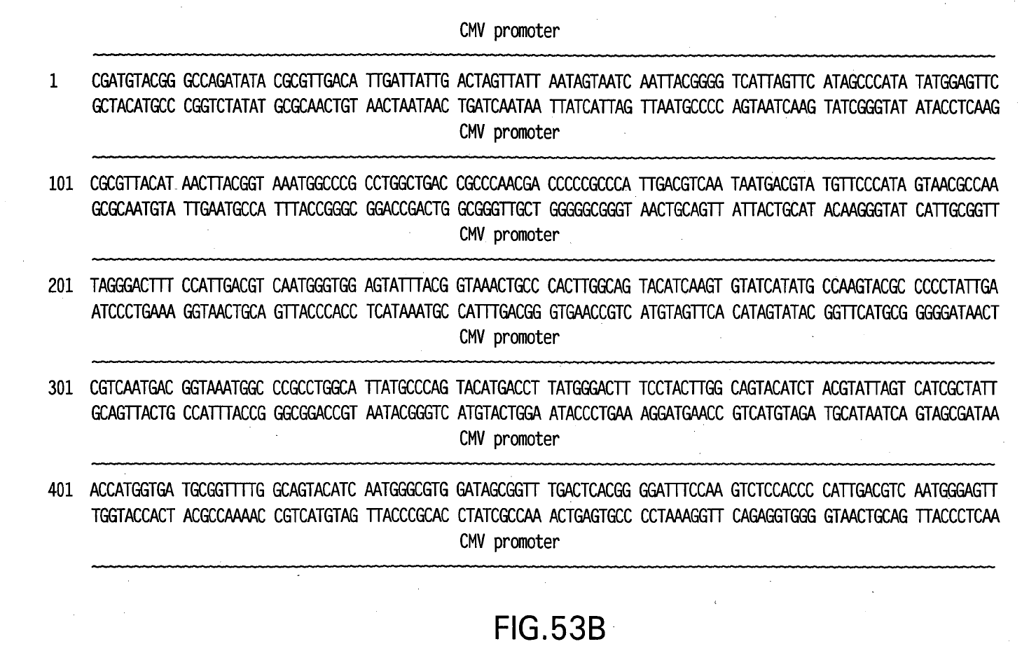

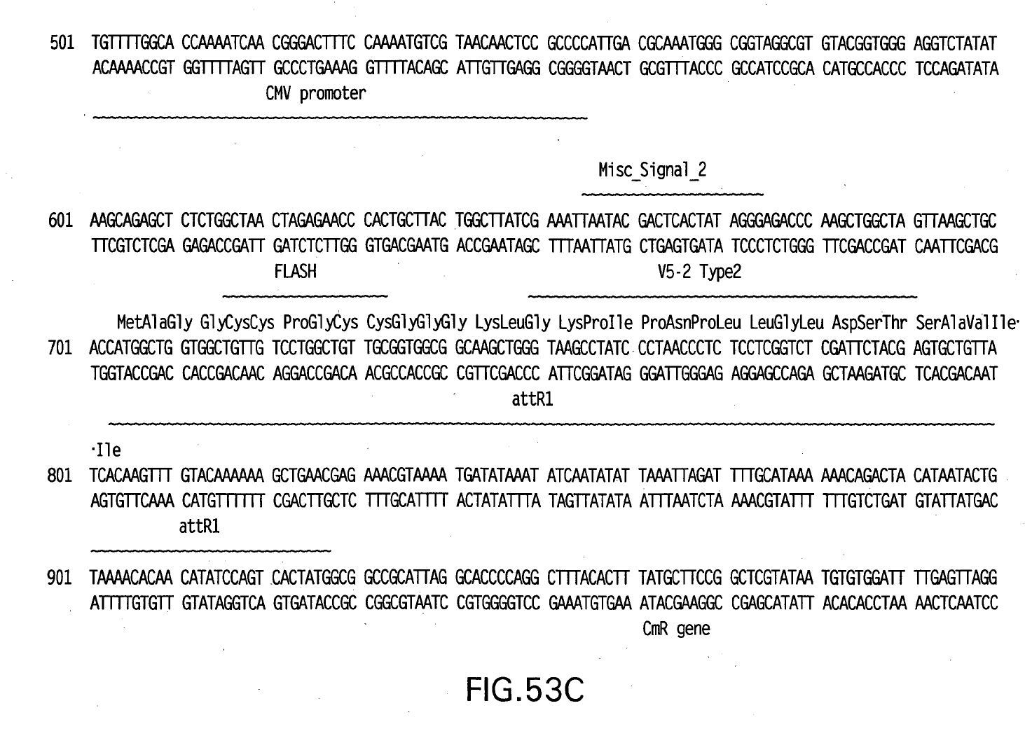

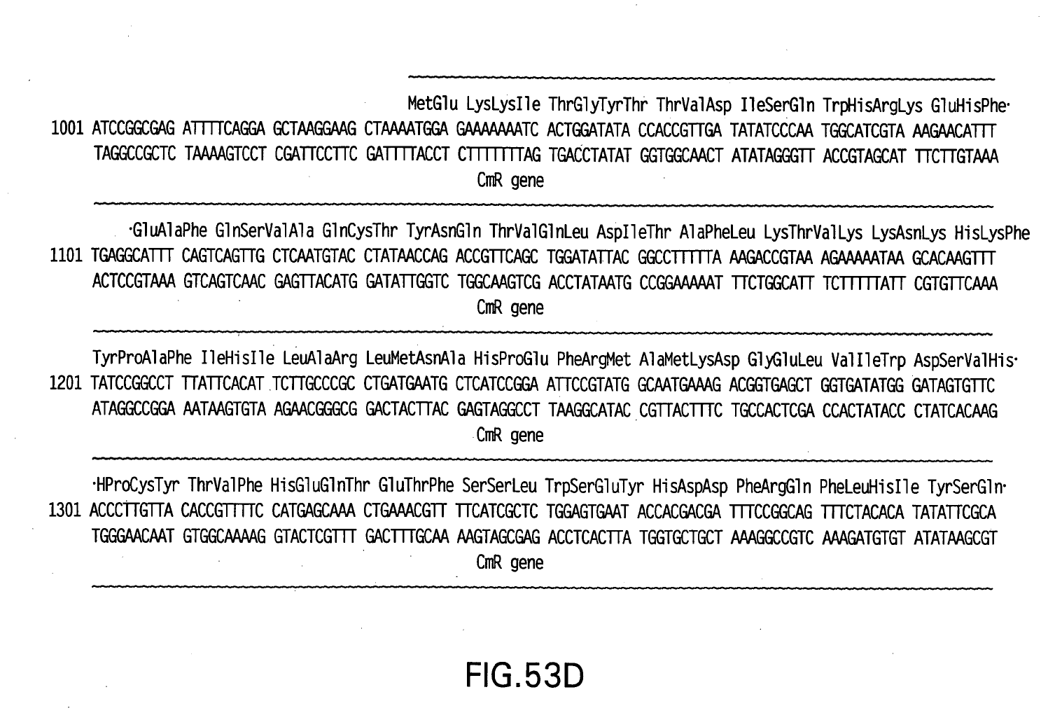

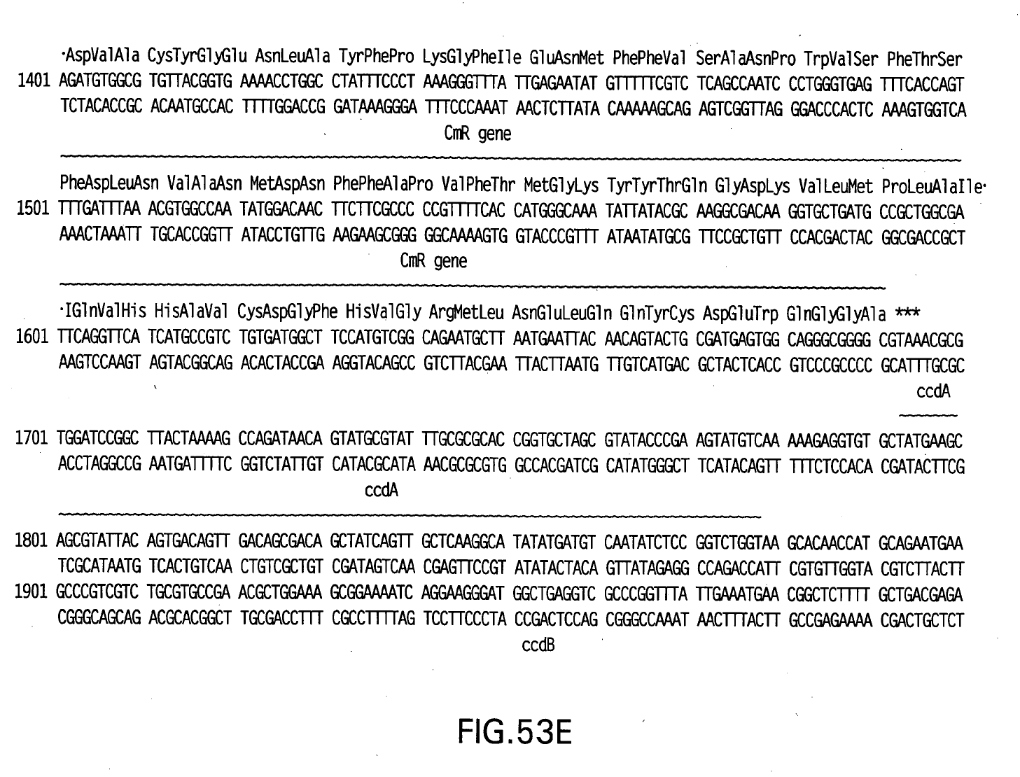

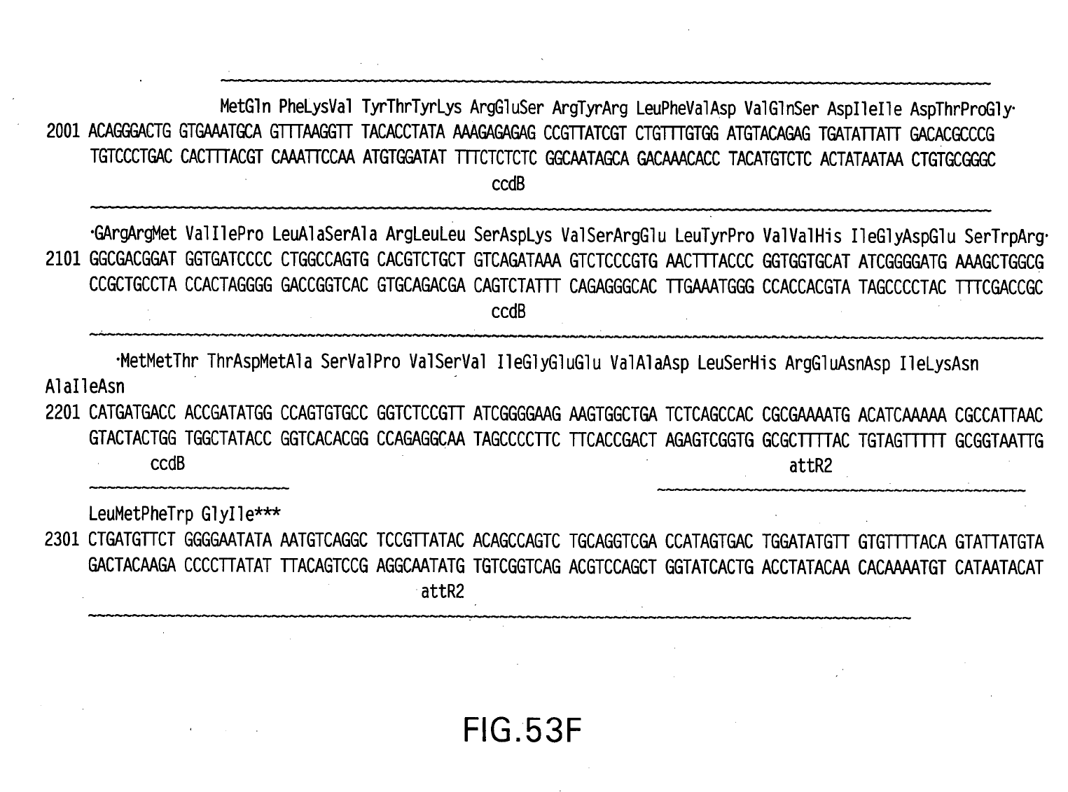

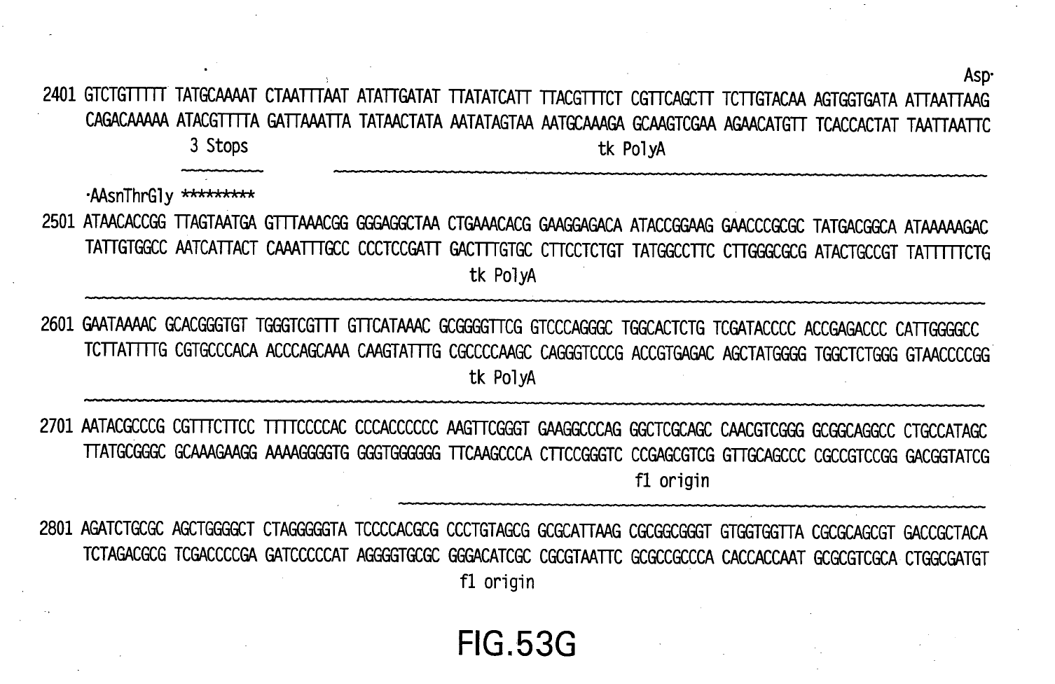

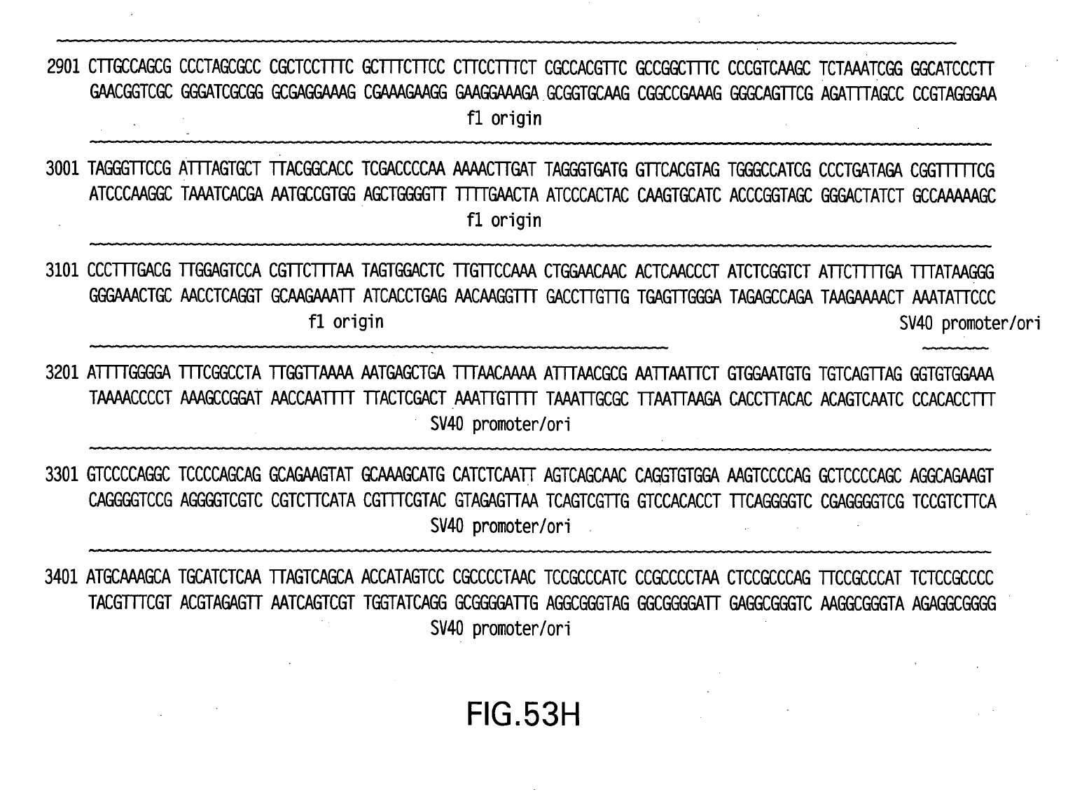

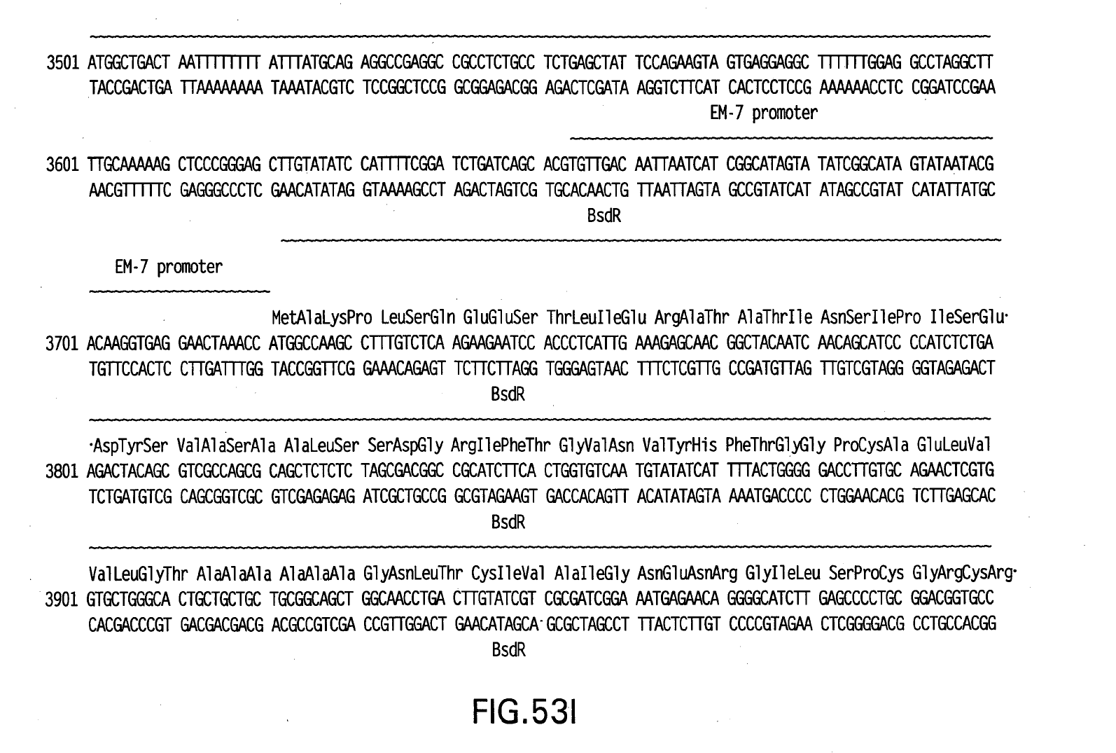

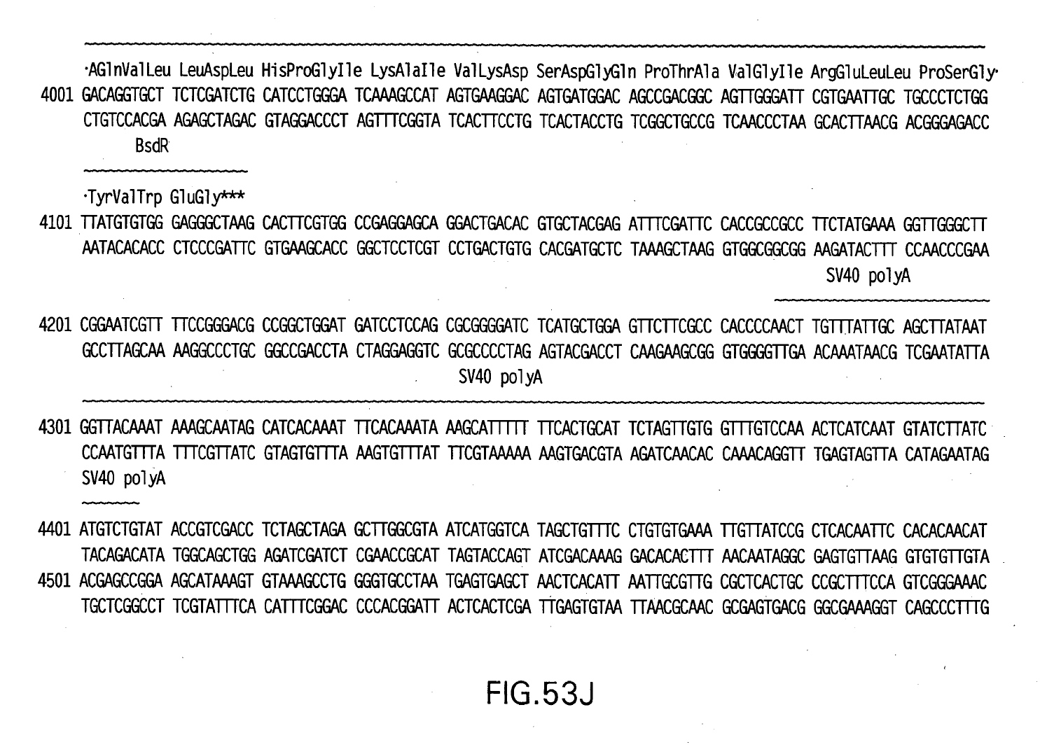

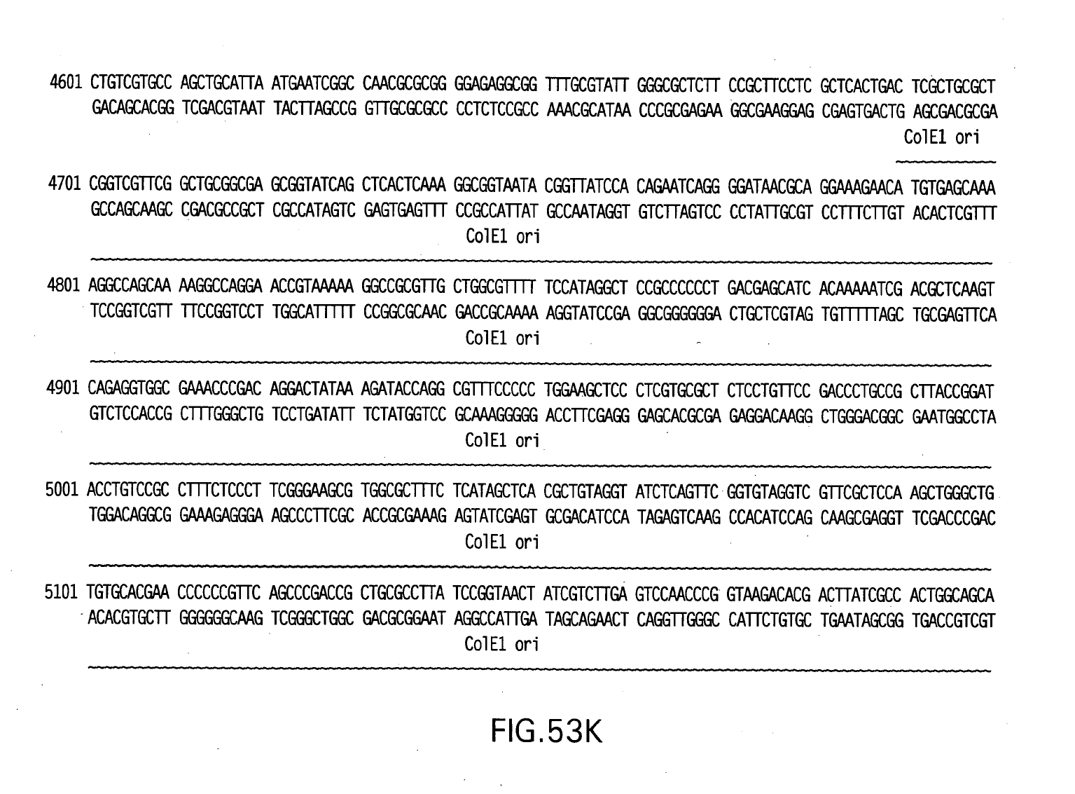

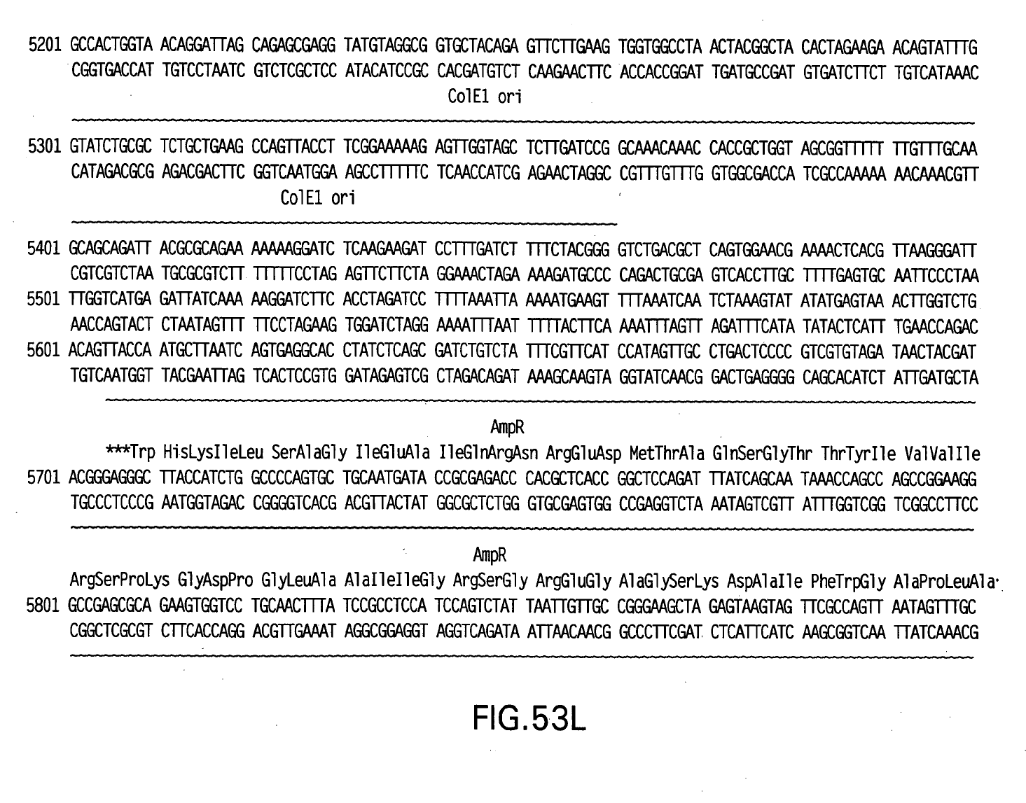

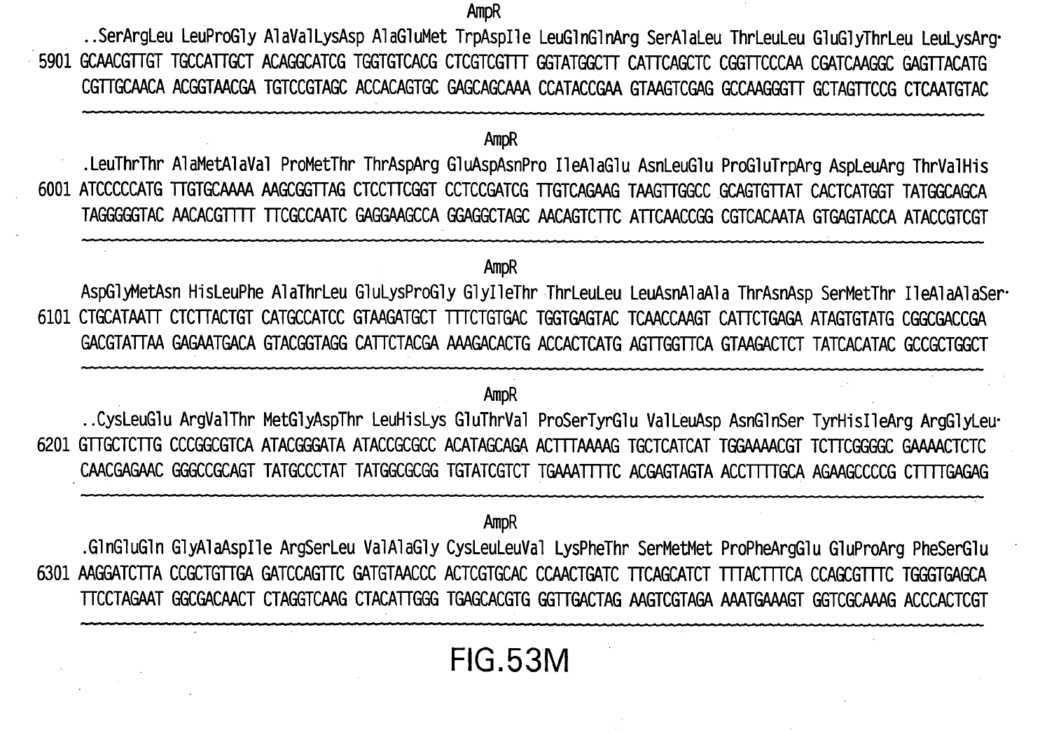

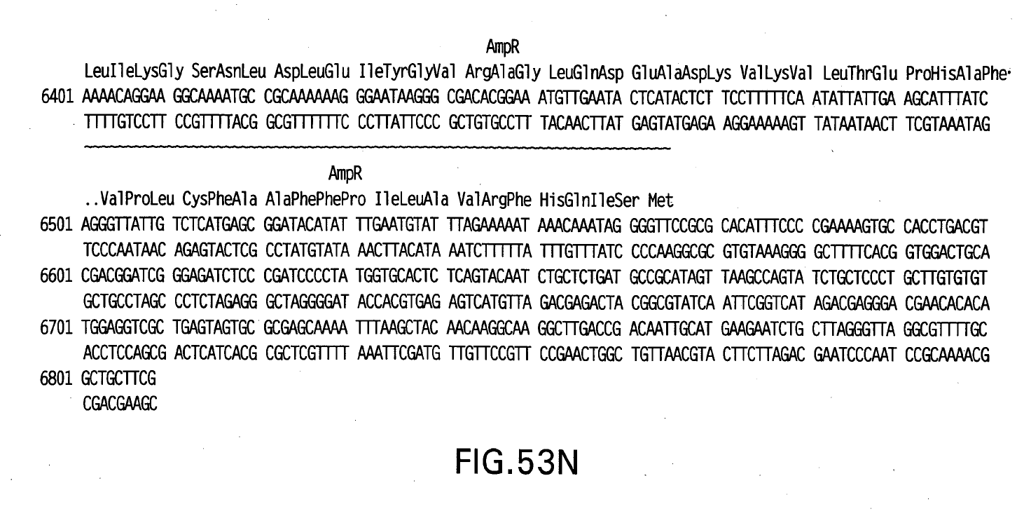

The vectors were prepared with the Qiagen Mega Prep kit from 500 ml LB with chloramphenicol. The pET160/LUMIO™-DEST and pET161/LUMIO™-DEST vectors were used in LR crosses with the entry vector pENTR-DT.2BaeIv2 ccdBDT (FIG. 53). This vector contained the TOPO™ site and ccdb gene flanked by an attLI and attL2 sites. The vector also contained two flanking Bael sites or NotI/AscI sites, both of which cleave the vector and allow for TOPO™ charging of the vector. The LR reactions were digested with EcoRI and transformed into DB3.1 cells and plated on LB ampicillin plates. Positive clones were sequenced.

TOPO™ Charging of pET160/D-TOPO™ and pET161/D-TOPO™.

The pET160 and 161 D-TOPO vectors were TOPO adapted essentially as follows:

Materials:

| | |
|---|---|
| 12° C. waterbath | Agarose gel apparatus and sterile 1 mm comb |
| 1X nuclease-free TAE | General Purpose Agarose |
| Buffer | |
| Nuclease-free TAE | Nuclease-free Medical Irrigation Water |
| Buffer pH 8.0 | |
| Nuclease-free 3M | Nuclease-free 80% Ethanol |
| Sodium Acetate pH 5.2 | |
| Isopropanol | Molecular Biology Grade Phenol/Chloroform |
| Ethanol | Supercoiled plasmid (Qiagen) |
| NotI, High concentration | AscI, High concentration |
| T4 DNA Ligase, High | 10X NEB Restriction Buffer #1 |
| concentration | |
| TopoD-74 oligo | TopoD-75 oligo |
| TopoD-90 oligo | TopoD-76 oligo |
| TopoD-70 oligo | 10X Stop Buffer |

Oligo Secquences:
TopoD-74 5′ PGGTGAAGGGGGC 3′
TopoD-75 5′ PCGCGCCCACCCTFITGACATAGTACAGTTG 3′
TopoD-76 5′ PAAGGGTGGG 3′
TopoD-90 5′ PGGCCGCCCCCTTCACCGACATAGTACAGTTG 3′
TopoD-70 5′ PCTGTACTATGTC 3′

Buffer Formulations:

10X Stop Buffer: 100 mM Tris 7.4, 110 mM EDTA, bromophenol blue
2X Wash Buffer: 60 mM Tris 7.4, 1 mM EDTA, 4 mM DTT,
200 μg/ml BSA, 100 mM bromophenol blue
Glycerol Mix: 90% Glycerol, 10% 50 mM TE pH 7.4 + 0.1%
Triton X-100

Final Vector Formulation: 10 ng/μl plasmid DNA in:
50% glycerol
50 mM Tris-HCl, pH 7.4 (at 25° C.)
1 mM EDTA
2 mM DTT
0.1% Triton X-100
100 μg/ml BSA
30 μM bromophenol blue

Procedure:

NotI/AscI Digest

100 μg of supercoiled DNA was digested with NotI using 6 Units/μg of DNA at a final vector concentration of 0.25 μg/ml. The mixture was then incubated in a 37° C. incubator for 3 hours with occasional mixing/spinning. The mixture was then extracted with ½ volume phenol/chloroform followed by precipitation with 1/10 volume 3M Sodium Acetate and 2× volume room temperature Ethanol. The pellet was then washed with 80% Ethanol. The pellet was then resuspended in nuclease-free water to a DNA concentration of 1 μg/μl and 1 μg was run on a 0.8% agarose gel to verify 100% digestion. Once complete digestion with NotI was confirmed, the linear DNA was digested with AscI as described above for NotI. Following AscI digestion and purification, the pellet was resuspended in nuclease-free water to a DNA concentration of 1 μg/μl.

Adaptor Ligation/Purification

Unless other wise stated, the following methods were performed at room temperature. With a final volume of 200 μl, quantities of the following components were calculated and added in the order listed below:

| Component | Amount Needed |
|---|---|
| pET160 or 161- | All of above |
| linearized | |
| M.I. Water | Adjust to final volume |
| TopoD-74 | 1.8 μg/10 μg vector |
| TopoD-75 | 4.0 μg/10 μg vector |
| TopoD-90 | 4.0 μg/10 μg vector |
| TopoD-76 | 1.2 μg/10 μg vector |
| 10X Ligation Buffer | 1X Final |
| | concentration |
| T4 DNA Ligase | ~2 Weiss units/10 μg |
| | vector |

The tube was inverted, spun briefly, and incubated at 12° C. overnight. The solution was then extracted with 1 volume of phenol chloroform and precipitated with 1/10 volume of 3M Sodium acetate and 2× volume Ethanol. The pellet was washed with 80% Ethanol and then resuspended in TE Buffer to a DNA concentration of 1 μg/μl. 1/10 volume 3M Sodium acetate and 0.7 volume Isopropanol was added at room temperature and the solution was mixed. The solution was then spun at high speed for 1 minute and the supernatant removed. The pellet was then resuspended in TE Buffer and twice reprecipitated as described above for a total of 2 isopropanol precipitations to ensure that excess adaptors were removed. The final pellet was then washed with 80% Ethanol. The pellet was then resuspended in TE Buffer to a DNA concentration of 1 μg/μl.

Topoisomerase Reaction and Gel Purification.

Unless other wise stated, the following methods were performed at room temperature. A 0.9% agarose gel containing a 1 mm thick comb was prepared. The comb was removed and a fresh solution of 1×TAE buffer was added to the reservoirs up to the top edges of the gel without allowing the buffer to touch the top of the gel or enter the wells.

TopoD-70 was added to the solution of linear DNA to a concentration of 0.325 μg/μg of linear DNA.

2.375 μl of 10×NEB Restriction Buffer #1 was then added per μg of linear DNA and the solution was mixed well.

The DNA concentration was adjusted to 0.042 μg/μl with Medical Irrigation water. Vaccinia Topoisomerase I enzyme was then added to a concentration of 1 μg/μg of linear DNA. The mixture was then incubated at 37° C. for 15 minutes with mixing once in the middle of the incubation. After 15 minutes, the reaction was stopped by adding 2.5 μl of 10× Stop Buffer per μg of linear DNA and brief mixing. The mixture was then spun and then the supernatant was collected.

The total volume of reaction was run on an agarose gel at 70 volts for 15 minutes until the bromophenol blue dye ran down about 12 inch into the gel. While the gel was running, a new sterile container was chilled on ice. The voltage was reversed and the gel was run backwards for 90 seconds, and then the power supply was turned off. The solution was removed from the wells and transferred to the pre-chilled container. 2× Wash Buffer (blue) was then added to the well and allowed to sit in the well for 1 minute.

The wells were then vigorously rinsed with the 2× Wash Buffer (blue) to resuspend any DNA trapped at the edges of the well and then transferred to the pre-chilled container. An equal volume of Glycerol Mix was added to the pre-chilled container and the solution was mixed gently by inversion to avoid the formation of bubbles. The solution was then spun and stored at −20° C. for 2 weeks or less, or at −80° C. for long term storage.

The prepared vectors were tested essentially as follows. Briefly the 750 bp BSD PCR Control was amplified using Pfu DNA Polymerase (Stratagene, La Jolla, Calif. 92037, cat. no. 600153). One microliter of the PCR reaction was used in a 6 μl directional TOPO™ reaction. Two microliters of the TOPO™ reaction were transformed into Top10 chemically competent cells. After a one hour incubation in 300p SOC shaking at 37° C., 100 μl was plated on LB/AMP plates. Colonies were counted to determine cloning efficiency and minipreps were checked by restriction analysis to determine percent of directional clones.

LR Recombination of Entry Clones with pET160-DEST and pET161-DEST.

Fusion protein expression constructs were generated using the pENTR kinase vectors. Kinase entry clones were obtained from the Ultimate ORF Collection. Constructs containing a stop codon and were used in the LR reaction with pET160-DEST. A pENTR-CAT construct, which is similar to the vectors of cat. nos. 12562-013 and 12562-039 sold by Invitrogen Corp, Carlsbad, Calif., containing a stop codon was also used to generate the GATEWAY® expression control plasmid. Constructs without a Shine-Dalgarno or stop codon were used in the LR reaction with pET161-DEST. These included a pENTR-CAT construct without a stop codon to create the C-terminal GW control plasmid. The LR reactions were set up using 4 μl of the LR Reaction Buffer, 2111 of the entry clone (50 μg/μl), 300 ng of the pET-DEST vector, and 4 μl of LR CLONASE™ Enzyme Mix (Invitrogen Corp, Carlsbad, Calif., cat. no. 11791-043) in a final volume of 20 μl. The reactions were incubated at 25° C. for 60 minutes. Then 211 of Proteinase K Solution was added to all reactions and incubated for 10 minutes at 37° C. One microliter of the reactions was used to transform 50 μl DH10B competent cells and plated on LB ampicillin. Colonies were minipreped and analyzed by restriction digest to confirm sequence and orientations. The LR cross of pET160-DEST was also transformed into TOP10 and Mach 1 competent cells.

D-TOPO™ Reaction with pET160/D-TOPO™ and pET161/D-TOPO™

Primers were designed to clone the CAT gene into the pET/D-TOPO™ vectors. The forward primers contained C/ACC/ATG sequence enabling directional cloning and an initiation codon. The dTOPO™CAT-STOP reverse primers had a TAG stop for cloning into the N-terminal vector. The dTOPOTMCAT-NS reverse primer did not contain a stop codon and allowed for translation of the C-terminal fusion. PCR products were amplified using the appropriate primers and 2.5 U Pfu Polymerase (Stratagene) in the 1×Pfu Buffer with 50 μM dNTP's for 25 cycles.

| | |
|---|---|
| DTOPO ™ CAT for | |
| 5′ CACCATGGAGAAAAAAATCACTGGATA 3′ | |
| DTOPO ™ CAT-STOP rev | |
| 5′ CTACGCCCGCCCTGCCACTCAT 3′ | (SEQ ID NO:141) |
| DTOPO ™ CAT-NS rev | |
| 5′ CGCCCCGCCCTGCCACTCATAGT 3′ | (SEQ ID NO:142) |

The PCR product was directionally TOPO™ cloned into the prepared pET-DEST160 D-TOPO™ vectors which were used to transform into DH5alpha cells and plated on plates containing LB agar plates containing ampicillin. Colonies were screened by DNA miniprep and restriction digests. A positive clone from each was transformed into BL21 Star cells for expression testing and are the positive control vectors for the kits.

BP Recombination of Expression clones with pDONR201 The pET160/CAT vector and pET161/Kinase C8 were used in BP reactions with pDONR201 (Invitrogen Corp, Carlsbad, Calif., cat. nos. 11798-014 and 11821-014) to regenerate entry clones. One hundred nanograms of each vector was linearized with XbaI for 1 hour at 37° C. The plasmid was gel purified and used in a 20 μl BP reaction with 300 ng pDONR201 (pET160 construct) and pDONR221 (pET161 construct) (Invitrogen Corp, Carlsbad, Calif., cat. nos. 12535-019 and 12536-017), 4 μl BP CLONASE™ Enzyme Mix (Invitrogen Corp, Carlsbad, Calif., cat. no. 11789-021), 1× Reaction Buffer and TE. After incubation at 25° C. for 1 hour, 211 of Proteinase K solution was added and incubated for 10 minutes at 37° C. One microliter of the reaction was used to transform DH5alpha competent cells and they were plated on LB agar plates containing 50 μg/ml kanamycin. Minipreps were performed on the resulting colonies and restriction analysis confirmed the fidelity of the recombination reactions.

Expression of Fusion Proteins in pET160-DEST and pET161-DEST.

The pET-DEST vectors carrying expression proteins were introduced into BL21 Star cells. Four ml LB ampicillin were innoculated with 20 μl of an overnight culture or with five colonies from an overnight plate. Cells were grown to A₆₀₀=0.4 and induced in 1 mM IPTG for three hours at 37° C. Cells (1 ml) were harvested by centrifugation, and resuspended in 0.1 ml of 1× Running Buffer. Cell lysates were prepared by equilibrating 10 ul of cell suspension to 50 mM FLASH™ Reagent (Invitrogen Corp, Carlsbad, Calif., cat. no. P3050) and IX Loading Dye (80 mM Tris, pH 6.8, 3% SDS, 15% Glycerol, 0.35 M beta-mercaptoethanol, and 300 μg/ml Bromphenol Blue) then heating to 96° C. for 3 minutes. Samples were analyzed by SDS-PAGE and detected by fluorescence, SIMPLYBLUE™ (Invitrogen Corp, Carlsbad, Calif., cat. no. LC6060) staining or immunoblotting.

Detection of Epitope Tags by Immunoblotting.

The mouse-anti-HisG or mouse-anti-His C antibody (Invitrogen Corp., cat. nos. R94025 and R93025) was used to detect epitope tags by immunoblotting (1:5000 dilution). After SDS/PAGE separation, proteins were blotted to nitrocellulose, and detected using the WESTERNBREEZE® Kit (Anti-Mouse) (Invitrogen, Corp, Carlsbad, Calif., cat. no. WB7104).

Purification of Recombinant Proteins by ProBond Metal Affinity Chromatography.

BL21 Star cells were transformed with pET160-GW/Kinase H5 (BC007462) or with pET161-GW/CAT. From a single colony, cells were grown overnight at 37° C., and inoculated 1/100 into 250 ml LB ampicillin. Cells were induced with 1 mM IPTG at A, =0.4. After a 3 hour induction at 37° C., cells were pelleted by centrifugation and lysed as described in the ProBond manual (Invitrogen Corp, Carlsbad, Calif., cat. no. K850-01) for denatured purification conditions at a 5× scale. The lysate (40 ml) was loaded onto a prepared ProBond (20 ml) column, and the column was gently agitated for 30 minutes to allow for binding. The resin was allowed to settle and the supernatant was aspirated off. The column was washed with Denaturing Binding Buffer by gentle mixing for 2 minutes. The resin was allowed to settle, the supernatant aspirated off, and the procedure repeated 1 more time. The column was washed twice with Denaturing Wash Buffer pH 6.0 of the ProBond kit and twice with Denaturing Wash Buffer pH 5.3. Protein was eluted by adding 10 ml Denaturing Elution Buffer of the ProBond kit. Two ml fractions were collected and monitored by SIMPLYBLUE™ staining.

Cleavage of MBP Produced by pET160-DEST by TEV Protease.

BL21 Star cells were transformed with pET160-GW/MBP. From a single colony, cells were grown overnight in LB ampicillin at 37° C., and inoculated 1/100 in 50 ml LB ampicillin. Cells were induced with 1 mM IPTG at A600=0.4. After a 3 hour induction. Cells were pelleted by centrifugation and were lysed as described in the ProBond manual for native purification conditions. The lysate (8 ml) was loaded onto a prepared native ProBond (4 ml) column, and the column was gently agitated for 20 minutes to allow for binding. The resin was allowed to settle and the supernatant was aspirated off. The column was washed with 8 ml of Native Wash Buffer by gentle mixing for 2 minutes. The resin was allowed to settle, the supernatant aspirated off and the procedure repeated 3 more times. The column was clamped in a vertical position and the cap snapped off the lower end. Protein was eluted by adding 8 ml Native Elution Buffer. One ml fractions were collected and monitored. The native substrate was used for digestion with TEV protease (Invitrogen Corp, Carlsbad, Calif., cat. no. 10127-017). Partially purified MBP was digested with TEV protease. Approximately 250 ng of protein isolated under native conditions was digested with 5 and 10 Units of TEV in 1×TEV Buffer for 3 hours at 37° C. Digested substrates were analyzed by SDS-PAGE (Novex, Tris-Glycine 4-20%).

In-Gel Detection of Protein in Lysates and Purified Proteins.

Protein lysates and purified protein samples were heated to 95-100° C. for 3 minutes in 1× Lanuneli Sample Buffer (Lammeli, V.K. 1970. Theor. Appl. Genet. 85:882-888) supplemented with 25CM FLASH™ substrate (Invitrogen Corp, Carlsbad, Calif., cat. no. P3050) (2 mM solution in 90% DMSO in water). The mixture was loaded onto 4-20% Tris-Glycine gel (Invitrogen) and separated by at SDS/PAGE. The gels were removed from the cassettes and visualized on standard UV boxes or by fluorescence imaging prior to SIMPLYBLUE™ staining.

GATEWAY® Adaptation and Expression of 96-Well Kinase Plate.

A 96-well plate containing 92 Ultimate ORF Kinase entry clones was used to generate expression clones and proteins in a high-throughput format. The remaining 4 wells were controls. A 96 LR cross of pENTR Kinase clones into pET160-DEST was directly transformed into BL21 Star cells and 25 μl of the transformation was used to inoculate 750 μl of LB ampicillin in a deep well 96-well plate. Overnight seed cultures were diluted 1:40 in fresh LB ampicillin (1.5 ml) and grown at 37° C. to induction at A₆₀₀=0.336 with 1 mM IPTG for 3 hours. Whole cell lysates were analyzed by SDS/PAGE as described above.

Results and Discussion

L×R Cloning and Expression Testing.

To demonstrate high-level expression from pET160-DEST, the vector was used in eight L×R reactions with pENTR-Kinases A1-H1 from the 96-well kinase plate. Positive clones were identified and transformed into BL21 Star cells for expression testing. The expression from lysates was seen in the SIMPLYBLUE™ stained gels (data not shown). Expression levels of the eight kinases from pET160-DEST were compared to the parental expression vector pET-DEST151 (which contains a V5 tag instead of the LUMIO™ tag). Protein levels were similar for both vectors, except for one case where less expression was observed for the LUMIO™ construct. The LUMIO™ tag also did not seem to adversely affect migration in SDS-PAGE gels as compared to the same protein containing the V5 tag. These results demonstrate that expression from the pET160-DEST vector is generally comparable to pET-DEST 151.

To test expression from pET161-DEST (the C-terminal LUMIO™ vector), the vector was used in a L×R cloning reaction with Entry clones containing CAT, kinase C8 (NM 004635) and D2 (BC000729) to create C-terminal fusion expression clones. As controls, pET-DEST42F, which is an intermediate vector made during the development of pET161-DEST (it contains a LUMIO™ tag in place of the V5 tag, but does not contain. a Shine-Dalgarno, start codon or T7 gene10 leader peptide) and pET161-DEST (ATG) (which contains the LUMIO™ tag, the Shine-Dalgarno and a start codon, but differs from pET161-DEST in that is does not contain the T7 gene10 leader peptide (amino acid sequence Met-Ala-Ser-Met-Thr-Gly-Gly-Gln-Gln-Met-Gly)) were used. The original parental vector pET42-DEST, which has the V5 tag rather than the LUMIO™ tag and pET-DEST42F were compared with each other by CAT and LacZ expression and verified to express equivalent levels of recombinant protein (data not shown). pET-DEST42F was used so that the expression could be compared when using the FLASH™ substrate and visualized by UV detection. CAT expression was better for the LUMIO™ construct, as compared to the pET-DEST42F and could be clearly seen by Coomassie staining. Additionally, these results demonstrated that the LUMIO™ tag and C-terminal His tag are both expressed properly as detected by western blot analysis using the appropriate antibodies. Therefore the pET161-DEST vector expresses at least as well as pET-DEST42.

When expressing non-E. coli genes, the kinases C8 and D2 both expressed significantly better from pET161-DEST and pET161-DEST (ATG) compared to the parental plasmid (data not shown). In fact, the kinases could not be detected by FLASH™ staining unless there was a translational leader attached to the N-terminus of the proteins. This may be because kinases with native N-termini are less stable or that the translational complexes do not initiate properly when expressing some native eukaryotic sequences. Regardless, both the T7 gene10 and the ATG translational leader sequences appeared to be effective at increasing expression, T7 gene10 for kinase C8 and ATG for kinase D2. However, it was decided that the T7 gene10 sequence would be used since it has been used successfully for a number of years in other pET vectors (Studier, F.W., Rosenberg, A. H., Dunn, J. J. and Dubendorff, J. W. (1990) Meth. Enzymol. 185:60-89). Therefore expression levels from pET161-DEST are satisfactory and the translational leader sequence appears to help expression of certain proteins.

The dTOPO™ (directional TOPO) Reaction and Expression Clone Induction

Both the pET-DEST/LUMIO™ constructs were TOPO™ charged to directly clone

PCR products. This allows those users who do not want to use the GATEWAY® pathway to directly create expression clones. The plasmids were TOPO™ adapted using the BaeI method. In brief, the plasmids were digested with Bael, oligonucleotide linkers with suitable topoisomerases recognition sequences were ligated to the ends, topoisomerase was added to vector, and the linkers were separated from the plasmids by gel electrophoresis. Expression clones were generated and tested for protein expression. The cloning efficiency for both the dTOPO™ reaction with the PCR Control was 99% and the directional efficiency was close to 80%. The vectors were also TOPO™ adapted using the NotI/AscI method as descibed above.

The CAT PCR product was directionally TOPO™ cloned into pET160/D-TOPO™. A positive clone was expressed in BL21 Star and compared to the pET160-GW/CAT construct. The cell lysate was detected by in-gel fluorescence, SIMPLYBLUE™, staining and by Western Blotting with and anti-His C antibody (FIG. 47, Panel A, B, and C). This demonstrates that the genes cloned into the attB D-TOPO™ vectors express similarly to genes cloned into pENTR. Interestingly, LUMIO™ tagged, FLASH™ labeled protein could be detected by UV exposure after transfer to the PVDF membrane (FIG. 47, Panel D).

### The BP Cloning Reaction

The functionality of the attB1 and attB2 sites of the pET160-GW/lacZ vector and pET161-GW/Kinase C8 were tested in BP reactions with pDONR201 or pDONR221 to regenerate entry clones. The BP reaction pET160-GW/lacZ into pDONR201 gave 171 cfu for the entire transformation in DH5 alpha chemically competent cells. Five of the six colonies screened by restriction digest were positive for the correct recombination. The (−) control gave 0 cfu for the entire transformation. The BP reaction of pET161-GW/Kinase C8 into pDONR221 resulted in approximately 1000 colonies and gave 6/9 positive for the correct recombination. The (−) control gave 42 cfu for the entire transformation. These results indicate that the attB sites of both vectors are functioning properly and can easily generate the desired entry clones.

Functionality of His tags in pET160 and pET161.

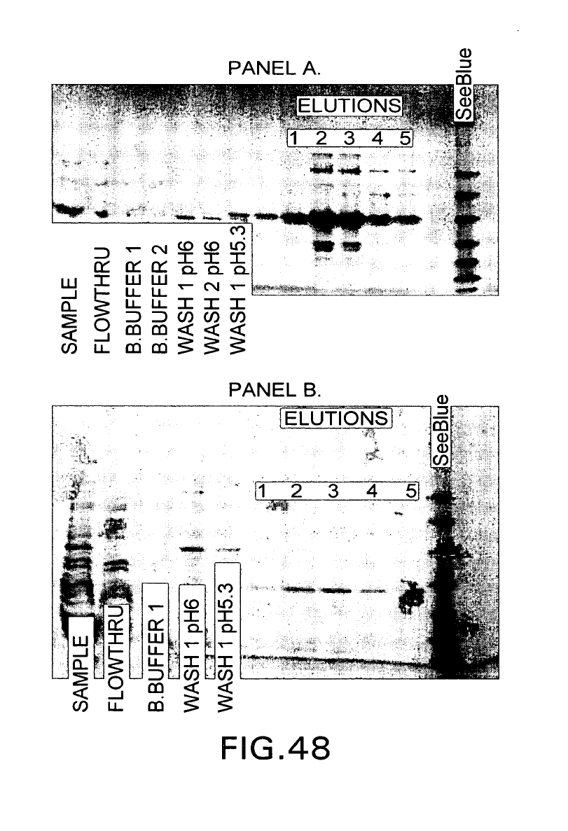

Both the HisG and C-terminal His antibodies recognize the His epitopes encoded by their respective vectors (data not shown). To verify that the His₆ tags functioned properly as purification epitopes, the pET161-GW/CAT and the pET160-GW/Kinase H5 (BC007462) construct were expressed in BL21 Star cells. The lysates were purified by Probond column using denaturing conditions and fractions from the lysate, washes and elution were analyzed (FIG. 48, panels A and B). The elution profiles showed the majority of the N-terminal LUMIO™-CAT protein eluting after 4 and 6 mls of denaturing elution buffer. For the C-terminal LUMIO™-Kinase H5 protein, a majority of the protein eluted after 2 and 3 ml of denaturing elution buffer. These results demonstrate that the His₆ purification epitope is functional.

Functional Testing of TEV Protease site in pET160.

To verify the recognition and proteolysis of the TEV cleavage site in the pET160 vector, the gene for Maltose Binding Protein (MBP) was crossed into pET160-DEST, expressed from BL21 Star and purified using ProBond nickel-chelating resin under native conditions. Approximately 250 ng of the partially purified protein was used in the reaction with TEV protease using standard conditions. In brief, MBP expression product was digested with either 5 or 10 Units of TEV protease for 3 hour digestion at 37° C. The digestion products we then analyzed by SDS-PAGE and compared against separate lanes which contained SEEBLUE® markers and partially purified His6/LUMIO™ tag/MBP protein. The results demonstrated that the pET160-DEST vector can encode a functional TEV cleavage site.

High Throughput Cloning, Expression, and In-Gel detection of the control 96-well Kinase Plate.

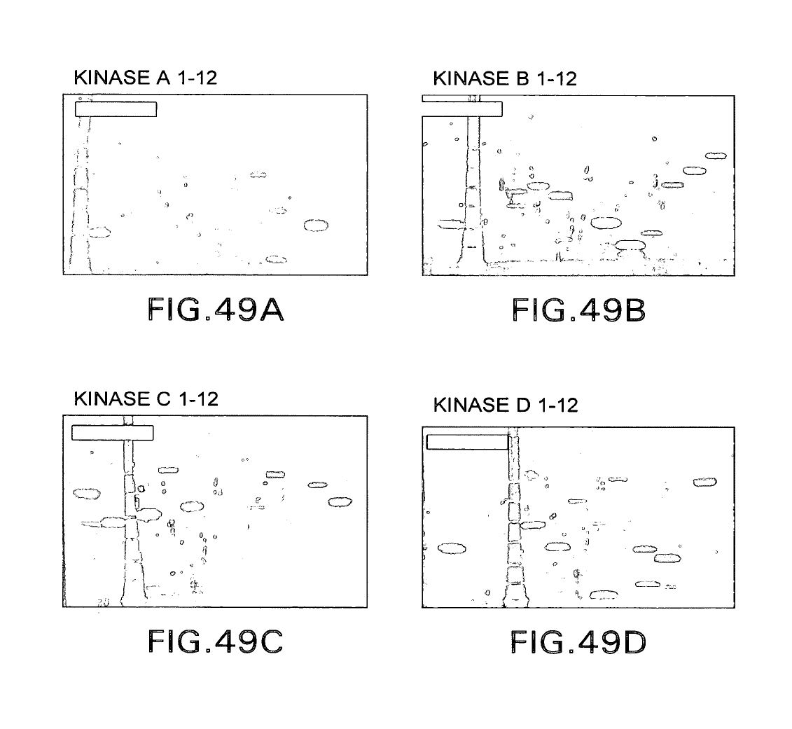

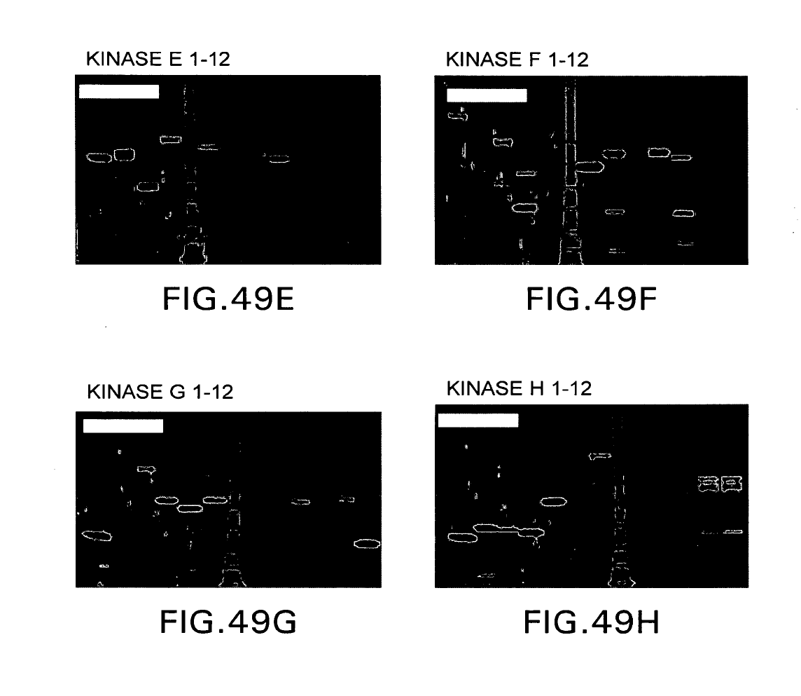

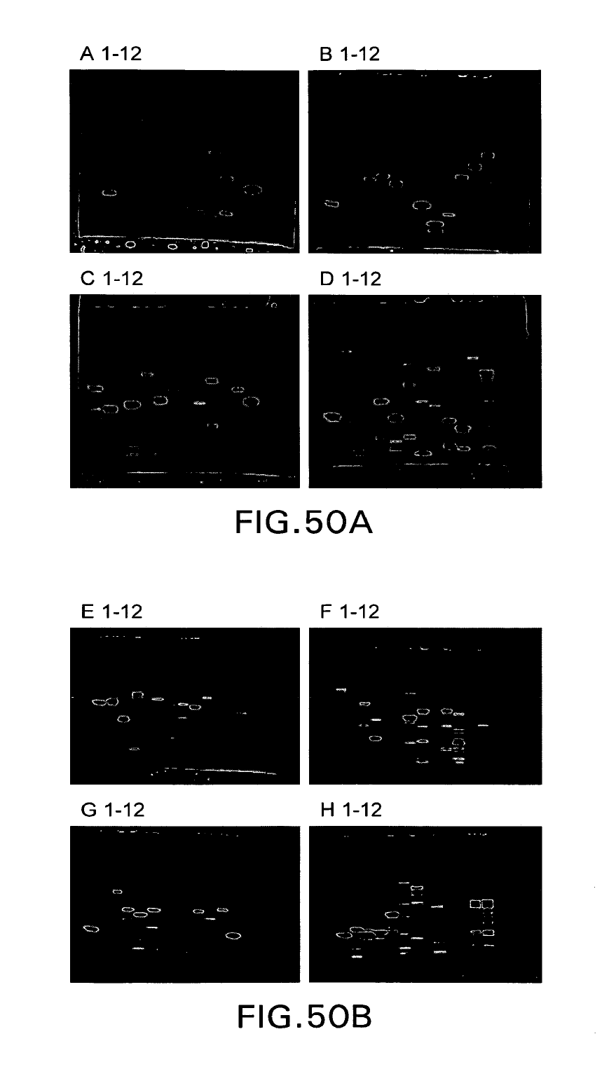

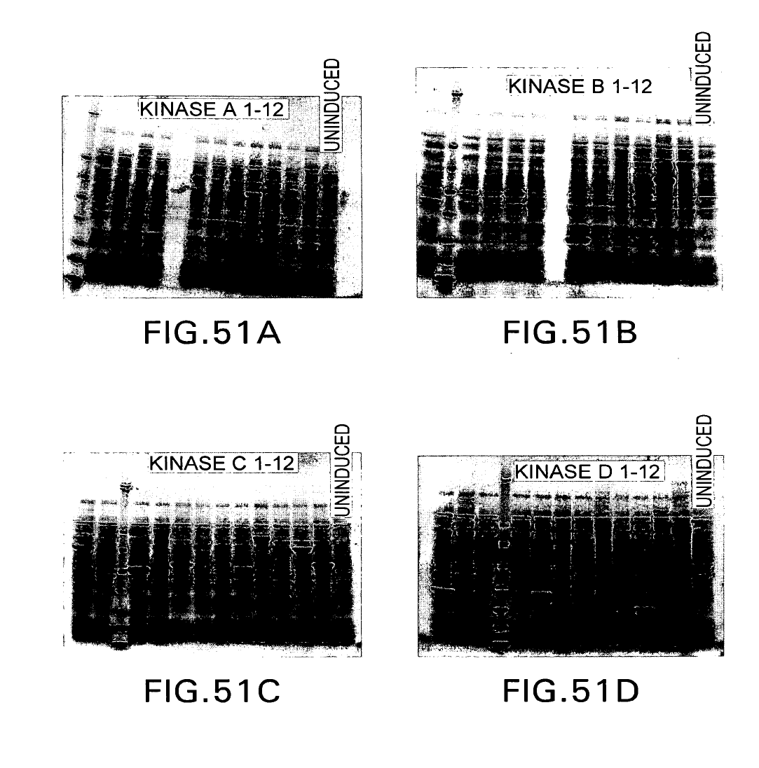

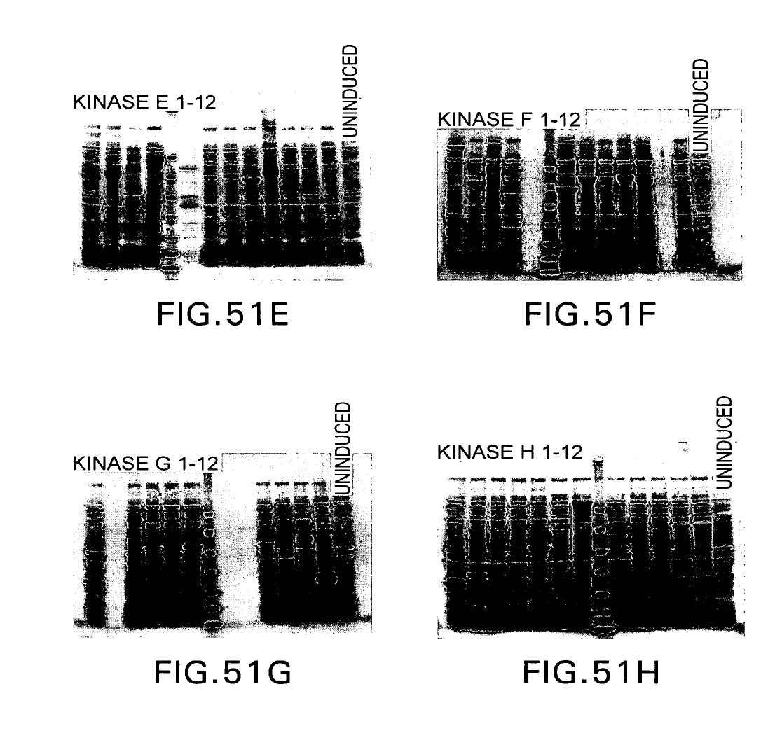

To demonstrate the utility of pET160-DEST in a high-throughput format, the vector was crossed with pENTR Human Kinase clones, which were stored in the individual wells of a 96-well plate, and then transformed directly into BL21 STAR cells for overnight growth. Wells G2, A5, F5, B6, G7, and F11 had little or no growth in the overnight cultures. After dilution into fresh media and growth at 37° C., the cultures were induced for 3 hours and lysates prepared from each well. The samples were reacted with FLASH™ substrate, separated on 4-20% SDS-PAGE gels and analyzed by UV fluorescence (FIG. 49) and the Typhoon imaging system (FIG. 50) which is a fluorescent detection instrument. 84% (69/82) of the kinases were detected at their predicted molecular weights. When the same gel was stained using SIMPLYBLUE™ Safe Stain 76% (62/82) of the kinases were detected at their respective molecular weights (FIG. 51). Identification of the expressed recombinant protein was significantly faster and easier using FLASH™ detection than with SIMPLYBLUE™ staining. Additionally, the Typhoon system was more sensitive than the standard UV box. Smears which were found to run down from the main protein band in each lane most likely represent degradation products or incomplete translation products. Also, each gel contained a bright fluorescent lane that was the SEEBLUE® molecular weight marker. High background fluorescence was observed which is due to the fluorescent dye that is a component of the marker.

Conclusions

We have validated the utility of GATEWAY® and d-TOPO™ cloning technology in generating LUMIO™ expression clones. Expression clones were induced using IPTG and found to express at levels comparable to existing pET vectors. A 96-well plate containing human kinase entry clones from the Ultimate ORF collection, referred to above, was used for high throughput cloning and expression using the GATEWAY® technology and FLASH™ detection. The epitope tags, purification and cleavage sequences perform as expected providing excellent functionality to the vectors. The TEV protease efficiently cleaves the N-terminal sequences from partially purified protein. The HisG and C-term His epitopes were detectable by using the appropriate antibody and not sterically hindered by the FLASH™ binding complex. Recombinant proteins were purified using the Probond column under standard native and denaturing conditions. The His₆ sequences may be removed.

Protein expression monitoring was greatly facilitated by in-gel detection of proteins containing the LUMIO™. This method of detection is quick and convenient, having virtually no additional processing. Much like EtBr staining of DNA samples, the FLASH™ substrate is simply added to the sample buffer prior to boiling, the samples run on SDS-PAGE gels and the labeled proteins visualized by UV light and EtBr filter (even while the gel is still in the cassette). An additional application is that transfer of the labeled proteins to a western blotting membrane can also be monitored under UV light. For the best results (most sensitivity), removing the gel from the plastic cassettes prior to visualization is recommended.

In a protein BLAST database homology search, it was observed that the LUMIO™ binding motif (CCPGCC) is rarely found in proteins, however the motif CCXXCC is more common and may contribute to some background staining. Some background staining is also likely due to protein breakdown and a large dye front that runs in the 3 kD range. Finally, some background is a result of the native E. coli protein Sly D, which contains a cluster of cysteines at the C-terminus of the protein.

The pET160/LUMIO™ and pET161/LUMIO™ vectors should be ideal for high-throughput cloning and expression of many different proteins. Researchers with a desire to clone and express many open reading frames for structural studies, antibody production, and other proteomic applications will find these vectors easy to use. Robotic liquid handling could be used to automate cloning and expression and now have the added advantage of rapid in-gel detection. In the future, the tetra cysteine LUMIO™ motif should be able to serve as a purification tag with all the advantages of the 6×His tag (small, purification using native and denaturing conditions, strong and specific binding).

### Example 9

### Introduction

LUMIO™ Green (also referred to herein as FLASH™) and LUMIO™ Red (also referred to herein as REASH™) may be used for in vivo protein labeling of mammalian cells. LUMIO™ Red is described for example in Adams S R, et al. (2002) J. Am. Chem. Soc. 124:6063-6076. Similar to LUMIO™ Green, LUMIO™ Red is non-fluorescent prior to binding the same tetracysteine motif (-CCXXCC-). After binding it has an excitation maximum of 593 nm and emission maximum at 608 nm, giving a red fluorescent color. This reagent allows for double labeling with LUMIO™ Green (Gaietta G. et al. (2002) Science 296:503-507) or use with other common green fluorescent molecules (e.g., GFP) in vivo.

Protocol for in vivo labeling of transfected mammalian cells is provided below.

### Materials And Methods

Materials:
Lipofectamine™ 2000 and protocol
GripTite™ 293 cells
DMEM+10% FBS growth media
6-well tissue culture plate
pcDNA6.2/nLUMIO™-GW/p64 DNA
OptiMEM (Invitrogen Corp., cat. nos. 11058-021, 31985-062, 31985-070, 31985-088, 51985-034)
LUMIO™ reagent, 2 mM in DMSO
Disperse Blue 3 stock solution, 20 mM in DMSO (see below for preparation instructions) (available from Sigma/Aldrich (catalog no. 21, 565-1) and Fisher (catalog no. AC20158-0500))
Fluorescent microscope with appropriate red and green filters (see below).

Preparation of 20 mM Disperse Blue Stock Solution:

Weigh 593 mg of Disperse Blue 3 powder (50 g, MW=296.32). Add 100 mL of dry DMSO to the Disperse Blue powder and mixed thoroughly by vortexing to dissolve. Pass solution through a 0.2 um filter.

To qualify the 20 mM Disperse Blue 3 stock solution, prepare 25 mL of 50% ethanol in water. Place 10 mL of the 50% ethanol solution in each of two 15 mL polypropylene tubes. Add 16.875 μl of the filtered 20 mM Disperse Blue 3 test sample prepared above to the first tube. This solution should be 3.375×10⁻⁵ M. Add 16.875 μl of 20 mM Disperse Blue 3 stock solution to the second tube. This solution is 3.375×10⁻⁵

M. Vortex each tube thoroughly. Using a spectrophotometer with a 1 cm path length, measure the absorbance of the diluted Disperse Blue 3 solutions at 638 nm, 593 nm and 256 nm wavelengths. Use quartz cuvettes and blank the instrument at each of these wavelengths with the extra 50% ethanol prepared above. Table 6 below provides the extinction coefficients for each wavelength and the range of absorbance values that should be obtained at each wavelength. These values will be used to evaluate the dye content of the test sample of Disperse Blue 3.

TABLE 6

| | 638 nm | 593 nm | 256 nm |
|---|---|---|---|
| Extinction | 6,600 | 5,300 | 13,000 |
| Coefficient | | | |
| Expected | 0.220 ± 0.022 | 0.175 ± 0.018 | 0.430 ± 0.043 |
| Absorbance | | | |
| Range | | | |

If the absorbance values of the Disperse Blue 3 do not fall within the range of expected absorbance values, repeat the qualification procedure described above.

If the absorbance values obtained for the test sample of Disperse Blue 3 fall below the expected absorbance range, this indicates that the dye content of the 20 mM stock solution is not concentrated enough and needs to be adjusted (see formula below).

(Absorbance)/(extinction coefficient)=concentration (in Molar units)
Eg. If absorbance 638 nm reads 0.180, then [0.180]/[6,600]=2.727×10⁻⁵ M 2.727×10⁻⁵ M is lower than the expected 3.375×10⁻⁵ M

Adjust accordingly and re-QC.

Conversely, if the absorbance values measured for the sample of Disperse Blue falls above the expected absorbance range, this indicates that the dye content of the 20 mM stock solution is too high and it needs to be diluted (see formula above).
Eg. If absorbance_{638 nm} reads 0.260, then [0.260]/[6,600]=3.939×10⁻⁵ M 3.939×10⁻⁵ M is higher than the expected 3.375×10⁻⁵ M
   Dilute accordingly and re-QC.

In vivo Labeling Protocol:

Day 1: Plate Cells For Transfection. Plate GripTite™ 293 cells into 6-well plates at 6×10⁵ cells per well. Four wells are used for every LUMIO™ test lot being evaluated (one “mock” well and one well in which a vector is to be introduced from which a protein or peptide with a LUMIO™ tag may be expressed, in duplicate).

Day 2: Transfection. Transfect cells with 4 μg of vector which expresses a protein or peptide with a LUMIO™ tag and 10 μL Lipofectamine™ 2000 per well exactly as described in the Lipofectamine™ 2000 protocol for 6-well plate. Make sure to do a mock transfection (no DNA, no lipid) for every LUMIO™ sample to evaluate background labeling.

Day 3: Change media on transfected cells to regular growth media.

Day 4: LUMIO™ label cells. Prepare 2 mL of 2.5 μM LUMIO™ labeling mix for each sample of LUMIO™ being evaluated, including the control sample. To prepare the labeling mix, add 2.5 μL of the 2 mM LUMIO™ stock to 2 mL OptiMEM. Vortex. Carefully remove medium from cells and rinse each well once with 2 mL OptiMEM. Carefully remove the OptiMEM and replace with 1 mL of LUMIO™ labeling mix per well. Label for 30 minutes at room temperature, in the dark. During the labeling, prepare 2 mL per well of 20 μM Disperse Blue in OptiMEM by adding 2 μL of the 20 mM stock for every 2 mL of OptiMEM. After the 30 minute labeling incubation, remove the labeling mix from the cells and discard it into an appropriate waste container. Carefully, rinse each well once with 2 mL OptiMEM, placing the rinse in the waste container. Gently add 2 mL 20 μM Disperse Blue in OptiMEM to each well.

Evaluate LUMIO™ labeled cells. Note: LUMIO™ Red photobleaches quickly; so visual and photographic evaluations must be performed quickly.
1. Turn UV source on (Mercury-100W box) and warm up for 10 minutes. Make sure the shutter to the UV source is in the closed position and the white light is off.
2. In order to properly view the fluorescent LUMIO™ labeling, the following filters are required:
   a. LUMIO™ Green: Excitation=508 nm, Emission=528 nm (standard FITC filters are suitable)
   b. LUMIO™ Red: Excitation=593 nm, Emission=608 nm (standard Texas Red filters are suitable)
3. The untransfected “mock” cells will appear lightly and uniformly green (or red, depending on which LUMIO™ reagent you are evaluating). The intensity of this “background” staining should be visually equivalent in both the “Gold Standard” and the test lots (see below for examples performed with qualified lots).
4. One example of a protein which may be expressed with a LUMIO™ tag is p64 (GenBank Accession No. BC000141, nucleolar c-myc variant). The p64 protein is localized to the nucleoli and should appear as discreet, brightly-labeled, punctate spots within the nuclei of cells upon use of the above procedures.

### Example 10

### Exemplary Product Instructions

The following example is intended to illustrate exemplary methods for carrying out the present invention. Variations on the methods set forth herein will be readliy appreciated by those skilled in the art. The information set forth in this or any other example should not be construed as limiting the scope of the invention described herein. All catalog numbers mentioned in this example refer to specific products and reagents avaliable from Invitrogen Corporation, Carlsbad, Calif., 92008. The exemplary methods described herein can be carried out using the products and reagents designated by the catalog numbers, or with equivalent products and reagents available from other sources.

TABLE 7
TOPO ® Cloning Procedure for Experienced Users

| Step | Action | | |
|---|---|---|---|
| Design PCR | Include the 4 base pair sequences (CACC) necessary for | | |
| Primers | directional cloning on the 5′ end of the forward primer. | | |
| | Design the primers such that your gene of interest will be | | |
| | optimally expressed and fused in frame with the β-lactamase | | |
| | reporter gene. | | |
| Amplify Your | 1. Use a thermostable, proofreading DNA polymerase and the | | |
| Gene of Interest | PCR primers above to produce your blunt-end PCR product. | | |
| | 2. Use agarose gel electrophoresis to check the integrity of your | | |
| | PCR product. | | |
| Perform the | 1. Set up the following TOPO ® Cloning reaction. For optimal | | |
| TOPO ® Cloning | results, use a 0.5:1 to 2:1 molar ratio of PCR product: TOPO ® | | |
| Reaction | vector. | | |
| | Note: If you plan to transform electrocompetent E. coli, use Dilute | | |
| | Salt Solution in the TOPO ® Cloning reaction. | | |
| | Fresh PCR product | 0.5 to 4 | μl |
| | Salt Solution | 1 | μl |
| | Sterile water | add to a final volume of 5 | μl |
| | TOPO ® vector | 1 | μl |
| | Total volume | 6 | μl |

| | |
|---|---|
| | 2. Mix gently and incubate for 5 minutes at room temperature. |
| | 3. Place on ice and proceed to transform One Shot ® Mach1 ™-T1^{R} |
| | chemically competent E. coli, below. |
| Transform | 1. Add 2 μl of the TOPO ® Cloning reaction into a vial of One |
| Mach1 ™-T1^{R} | Shot ® Mach1 ™-T1^{R} chemically competent E. coli and mix gently. |
| Chemically | 2. Incubate on ice for 5 to 30 minutes. |
| Competent | 3. Heat-shock the cells for 30 seconds at 42° C. without shaking. |
| E. coli | Immediately transfer the tube to ice. |
| | 4. Add 250 μl of room temperature S.O.C. medium. |
| | 5. Incubate at 37° C. for 1 hour with shaking. |
| | 6. Spread 50-200 μl of bacterial culture on a prewarmed selective |
| | plate and incubate at 37° C. Visible colonies should appear within |
| | 8 hours for ampicillin selection. Incubate plates overnight, if |
| | desired. |

TABLE 8
Types of Kits

| | Invitrogen |
|---|---|
| Product | Catalog no. |
| GeneBLAzer ™ C-terminal TOPO ® Fusion | 12578-076 |
| Kit for In Vitro Detection | |
| GeneBLAzer ™ C-terminal TOPO ® Fusion | 12578-084 |
| Kit for In Vivo Detection | |
| GeneBLAzer ™ N-terminal TOPO ® Fusion | 12578-092 |
| Kit for In Vitro Detection | |
| GeneBLAzer ™ N-terminal TOPO ® Fusion | 12578-100 |
| Kit for In Vivo Detection | |

TABLE 9
Kit Components

| | Invitrogen Catalog no. | | | |
|---|---|---|---|---|
| | 12578- | 12578- | 12578- | 12578- |
| Component | 076 | 084 | 092 | 100 |
| GeneBLAzer ™ | ✓ | ✓ | | |
| TOPO ® Reagents with | | | | |
| pcDNA6.2/cGeneBLAzer-GW/D- | | | | |
| TOPO ® | | | | |
| GeneBLAzer ™ TOPO ® | | | ✓ | ✓ |
| Reagents with | | | | |
| pcDNA6.2/nGeneBLAzer-GW/D- | | | | |
| TOPO ® | | | | |
| One Shot ® Mach1 ™- | ✓ | ✓ | ✓ | ✓ |
| T1^{R} Chemically Competent E. coli | | | | |
| GeneBLAzer ™ In | ✓ | | ✓ | |
| Vitro Detection Kit | | | | |
| GeneBLAzer ™ In | | ✓ | | ✓ |
| Vivo Detection Kit | | | | |

TABLE 10
Shipping and Storage

| Box | Item | Shipping | Storage |
|---|---|---|---|
| 1 | GeneBLAzer ™ TOPO ® | Dry ice | −20° C. |
| | Reagents | | |
| 2 | One Shot ® Mach1 ™- | Dry ice | −80° C. |
| | T1^{R} Chemically | | |
| | Competent E. coli | | |
| 3a | GeneBLAzer ™ In Vitro | Dry ice | CCF2-FA: −20° C., |
| | Detection Kit | | dessicated and |
| | | | protected from |
| | | | light |
| 3b | GeneBLAzer ™ In Vivo | Room | CCF2-AM: −20° C., |
| | Detection Kit | temperature | dessicated |
| | | | and protected |
| | | | from light |
| | | | Solutions: Room |
| | | | temperature, |
| | | | protected from |
| | | | light |

TABLE 11
GeneBLAzer ™ TOPO ® Reagents

| Item | Concentration | Amount |
|---|---|---|
| GeneBLAzer ™ vector, | 15-20 ng/μl linearized plasmid | 20 μl |
| linearized and TOPO ®-adapted | DNA in: | |
| (pcDNA6.2/cGeneBLAzer- | 50% glycerol | |
| GW/D-TOPO ® or | 50 mM Tris-HCl, pH 7.4 (at | |
| pcDNA6.2/nGeneBLAzer- | 25° C.) | |
| GW/D-TOPO ®) | 1 mM EDTA | |
| | 2 mM DTT | |
| | 0.1% Triton X-100 | |
| | 100 μg/ml BSA | |
| | 30 μM bromophenol blue | |
| dNTP Mix | 12.5 mM dATP | 10 μl |
| | 12.5 mM dCTP | |
| | 12.5 mM dGTP | |
| | 12.5 mM dTTP | |
| | in water, pH 8 | |
| Salt Solution | 1.2 M NaCl | 50 μl |
| | 0.06 M MgCl₂ | |
| Sterile Water | — | 1 ml |
| T7 Promoter Primer | 0.1 μg/μl in TE Buffer, pH 8 | 20 μl |
| (supplied with Catalog | | |
| nos. 12578-076 | | |
| and 12578-084 only) | | |
| TK polyA Reverse Primer | 0.1 μg/μl in TE Buffer, pH 8 | 20 μl |
| (supplied with Catalog | | |
| nos. 12578-092 | | |
| and 12578-100 only) | | |
| Control PCR Primers | 0.1 μg/μl each in TE Buffer, | 10 μl |
| | pH 8 | |
| Control PCR Template | 0.1 μg/μl in TE Buffer, pH 8 | 10 μl |
| Control Plasmid | 0.5 μg/μl in TE, pH 8.0 | 10 μl |
| (pcDNA ™ 6.2/cGeneBLAzer- | | |
| GW/lacZ or | | |
| pcDNA ™ 6.2/nGeneBLAzer- | | |
| GW/lacZ) | | |

TABLE 12
One Shot ® Mach1 ™-T1^{R} Reagents

| | | |
|---|---|---|
| Item | Composition | Amount |

| | | | |
|---|---|---|---|
| S.O.C. Medium | 2% Tryptone | 6 | ml |
| (may be stored at room | 0.5% Yeast Extract | | |
| temperature or +4° C.) | 10 mM NaCl | | |
| | 2.5 mM KCl | | |
| | 10 mM MgCl₂ | | |
| | 10 mM MgSO₄ | | |
| | 20 mM glucose | | |
| Mach1 ™-T1^{R} Cells | — | 21 × 50 | μl |
| pUC19 Control DNA | 10 pg/μl in 5 mM Tris-HCl, | 50 | μl |
| | 0.5 mM EDTA, pH 8 | | |

The Genotype of Mach1™-T1® Cells is as follows: F⁻ φ80(lacZ)ΔM15 ΔlacX74 hsdR(r_{K}⁻m_{K}⁺) ΔrecA1398 endA1 tonA Use this strain for cloning. Note that this strain cannot be used for single-strand rescue of DNA.

TABLE 13
Sequencing Primers

| Primer | Sequence |
|---|---|
| T7 Promoter Primer | 5′-TAATACGACTCACTATAGGG-3′ |
| (Catalog nos. 12578-076 | (SEQ ID NO: 143) |
| and 12578-084 only) | |
| TK polyA Reverse Primer | 5′-CTTCCGTGTTTCAGTTAGC-3′ |
| (Catalog nos. 12578-092 | (SEQ ID NO: 144) |
| and 125 78-100 only) | |

Additional products that may be used with the GeneBLAzer™ TOPO® Fusion Kits are available from Invitrogen and are listed in Table 14.

TABLE 14
Additional Products

| Item | Quantity | Catalog no. |
|---|---|---|
| GeneBLAzer ™ In Vitro Detection Kit | 100 μg | 12578-126 |
| GeneBLAzer ™ In Vivo Detection Kit | 50 μg | 12578-134 |
| One Shot ® Mach1 ™-T1^{R} Chemically | 20 reactions | C4040-03 |
| Competent E. coli | | |
| One Shot ® TOP10 Chemically | 10 reactions | C4040-10 |
| Competent E. coli | 20 reactions | C4040-03 |
| One Shot ® TOP10 Electrocompetent | 10 reactions | C4040-50 |
| E. coli | | |
| S.O.C. Medium | 10 × 10 ml | 15544-034 |
| PureLink ™ HQ Mini Plasmid | 100 reactions | K2100-01 |
| Purification Kit | | |
| Gateway ® BP Clonase ™ Enzyme Mix | 20 reactions | 11789-013 |
| | 100 reactions | 11789-021 |
| pDONR ™ 221 | 6 μg | 12213-013 |
| pDONR ™/Zeo | 6 μg | 12536-017 |
| Lipofectamine ™ 2000 | 0.75 ml | 11668-027 |
| | 1.5 ml | 11668-019 |
| Blasticidin | 50 mg | R210-01 |
| β-Gal Assay Kit | 100 reactions | K4155-01 |
| β-Gal Staining Kit | 1 kit | K1465-01 |
| β-Gal Antiserum* | 50 μl | R901-25 |

Introduction

The GeneBLAzer™ TOPO® Fusion Kits provide a highly efficient, 5-minute cloning strategy (“TOPO® Cloning”) to directionally clone a blunt-end PCR product into a reporter vector for expression in mammalian cells. The pcDNA6.2/GeneBLAzer-GW/D-TOPO® vector supplied with each kit facilitates in vivo or in vitro detection of β-lactamase reporter activity in mammalian cells using the GeneBLAzer™ Technology. Use of the GeneBLAzer™ Technology provides a highly sensitive and accurate method to quantitate gene expression in mammalian cells.

The pcDNA6.2/GeneBLAzer-GW/D-TOPO® vectors also allow easy transfer of your gene of interest into multiple vector systems using Gateways Technology.

Features of the pcDNA 6.2/GeneBLAzer-GW/D-TOPO® Vectors

The pcDNA6.2/cGeneBLAzer-GW/D-TOPO® and pcDNA6.2/nGeneBLAzer-GW/D-TOPO® vectors contain the following elements:

Human cytomegalovirus immediate-early (CMV) promoter/enhancer for high-level expression in a wide range of mammalian cells;

β-lactamase bla(M) reporter gene for C-terminal (pcDNA6.2/cGeneBLAzer-GW/D-TOPO®) or N-terminal (pcDNA6.2/nGeneBLAzer-GW/D-TOPO®) fusion to the gene of interest;

attB1 and attB2 sites for site-specific recombination of the expression clone with a Gateway® donor vector to generate an entry clone;

Directional TOPO® Cloning site for rapid and efficient directional cloning of blunt-end PCR products;

The V5 epitope tag for detection using Anti-V5 antibodies (pcDNA6.2/nGeneBLAzer-GW/D-TOPO® only);

The Herpes Simplex Virus thymidine kinase polyadenylation signal for proper termination and processing of the recombinant transcript;

f1 intergenic region for production of single-strand DNA in F plasmid-containing E. coli;

SV40 early promoter and origin for expression of the Blasticidin resistance gene and stable propagation of the plasmid in mammalian hosts expressing the SV40 large T antigen;

Blasticidin resistance gene for selection of stable cell lines;

The pUC origin for high copy replication and maintenance of the plasmid in E. coli;

The ampicillin resistance gene for selection in E. coli.

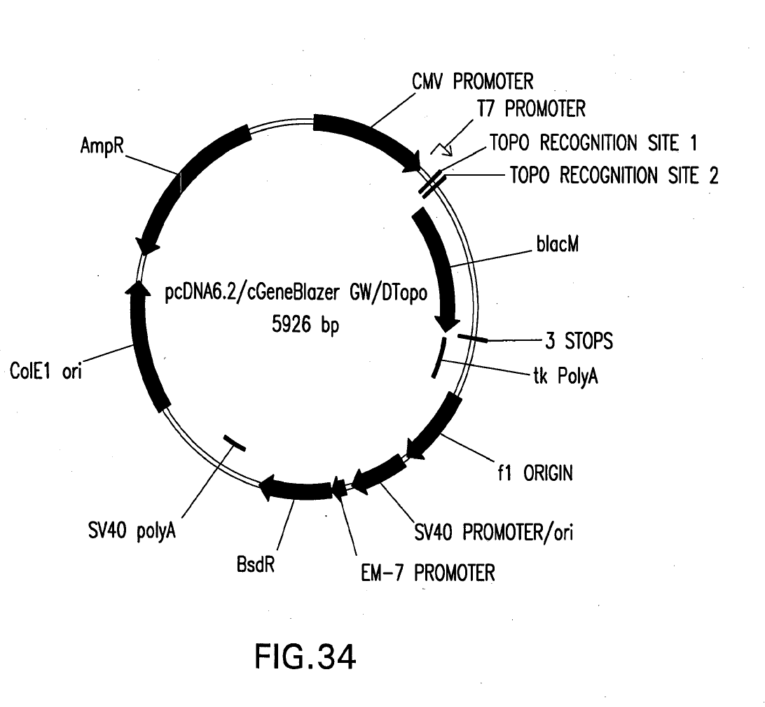

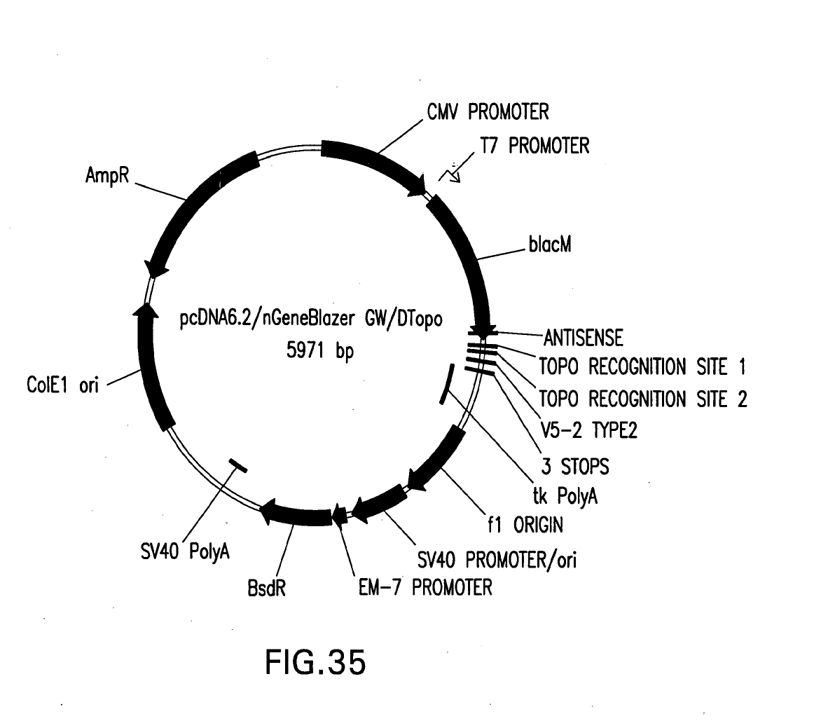

For a map of pcDNA6.2/cGeneBLAzer-GW/D-TOPO® or pcDNA6.2/nGeneBLAzer-GW/D-TOPO®, see FIGS. 34 and 35, respectively.

The Gateway® Technology

The Gateway® Technology is a universal cloning method that takes advantage of the site-specific recombination properties of bacteriophage lambda (Landy, A. (1989). Dynamic, Structural, and Regulatory Aspects of Lambda Site-specific Recombination. Annu. Rev. Biochem. 58, 913-949) to provide a rapid and highly efficient way to move your gene of interest into multiple vector systems. To express your gene of interest in mammalian cells, simply TOPO® Clone your blunt-end PCR product into a GeneBLAzer™ Directional TOPO® vector and transfect your expression clone into the mammalian cell line of choice.

To express your gene of interest in any other expression system:

1. Generate an entry clone by performing a BP recombination reaction between your expression clone and a Gateway® donor vector.

2. Perform an LR recombination reaction between the entry clone and a variety of Gateway™ destination vectors to generate an expression construct to express your protein of interest in virtually any expression system.

Advantages of the GeneBLAzer™ Technology

Using the GeneBLAzer™ Technology and the GeneBLAzer™ Detection System as a reporter of gene expression in mammalian cells provides the following advantages:

Suitable for use as a sensitive reporter of gene expression in living mammalian cells using fluorescence microscopy.

Provides a ratiometric readout to minimize differences due to variability in cell number, substrate concentration, fluorescence intensity, and emission sensitivity.

Compatible with a wide variety of in vivo and in vitro applications including microplate-based transcriptional assays and flow cytometry.

Provides a flexible and simple assay development platform for gene expression in mammalian cells.

Using a non-toxic substrate allows continued cell culturing after quantitative analysis.

One Shot® Mach1™-T1® E. coli

The Mach1™-T1®E. coli strain is modified from the wild-type W strain (ATCC #9637, S. A. Waksman) and has a faster doubling time compared to other standard cloning strains. With Mach^{T1}-T1® cells, you can visualize colonies 8 hours after plating on ampicillin selective plates. You can also prepare plasmid DNA 4 hours after inoculating a single, overnight-grown colony in the selective media of choice. Note that this feature is not limited to ampicillin selection.

Additional features of the Mach1™-T1® E. coli strain include:

lacZΔM15 for blue/white color screening of recombinants;

hsdR mutation for efficient transformation of unmethylated DNA from PCR applications;

ΔrecA1398 mutation for reduced occurrence of homologous recombination in cloned DNA;

endA1 mutation for increased plasmid yield and quality;

tonA mutation to confer resistance to T1 and T5 phage.

Tag-On-Demand™ System

The pcDNA6.2/GeneBLAzer-GW/D-TOPO® vectors are compatible with the Tag-On-Demand System which allows expression of both native and C-terminally-tagged recombinant protein from the same expression construct.

The System is based on stop suppression technology originally developed by RajBhandary and colleagues (Capone, J. P., Sharp, P. A., and RajBhandary, U. L. (1985). Amber, Ochre and Opal Suppressor tRNA Genes Derived from a Human Serine tRNA Gene. EMBO J. 4, 213-221) and consists of a recombinant adenovirus expressing a tRNAₛₑᵣ suppressor. When an expression vector encoding a gene of interest with the TAG (amber stop) codon is transfected into mammalian cells and the tRNAₛₑᵣ suppressor supernatant is present, the stop codon will be translated as serine, allowing translation to continue and resulting in production of a C-terminally-tagged fusion protein.

How Directional TOPO® Cloning Works

How Topoisomerase I Works

Topoisomerase I from Vaccinia virus binds to duplex DNA at specific sites (CCCTT) and cleaves the phosphodiester backbone in one strand (Shuman, S. (1991). Recombination Mediated by Vaccinia Virus DNA Topoisomerase I in Escherichia coli is Sequence Specific. Proc. Natl. Acad. Sci. USA 88, 10104-10108). The energy from the broken phosphodiester backbone is conserved by formation of a covalent bond between the 3′ phosphate of the cleaved strand and a tyrosyl residue (Tyr-274) of topoisomerase I.

The phospho-tyrosyl bond between the DNA and enzyme can subsequently be attacked by the 5′ hydroxyl of the original cleaved strand, reversing the reaction and releasing topoisomerase (Shuman, S. (1994). Novel Approach to Molecular Cloning and Polynucleotide Synthesis Using Vaccinia DNA Topoisomerase. J. Biol. Chem. 269, 32678-32684). TOPO® Cloning exploits this reaction to efficiently clone PCR products.

Directional TOPO® Cloning

Directional joining of double-strand DNA using TOPO®-charged oligonucleotides occurs by adding a 3′ single-stranded end (overhang) to the incoming DNA (Cheng, C., and Shuman, S. (2000). Recombinogenic Flap Ligation Pathway for Intrinsic Repair of Topoisomerase IB-Induced Double-Strand Breaks. Mol. Cell. Biol. 20, 8059-8068). This single-stranded overhang is identical to the 5′ end of the TOPO®-charged DNA fragment. At Invitrogen, this idea has been modified by adding a 4 nucleotide overhang sequence to the TOPO®-charged DNA and adapting it to a ‘whole vector’ format.

In this system, PCR products are directionally cloned by adding four bases to the forward primer (CACC). The overhang in the cloning vector (GTGG) invades the 5′ end of the PCR product, anneals to the added bases, and stabilizes the PCR product in the correct orientation. Inserts can be cloned in the correct orientation with efficiencies equal to or greater than 90%. See FIG. 6.

The GeneBLAzer™ Technology

Components of the GeneBLAzer™ System

The GeneBLAzer™ System facilitates fluorescence detection of β-lactamase reporter activity in mammalian cells, and consists of two major components:

The β-lactamase reporter gene, bla(M), a truncated form of the E. coli bla gene. When fused to a gene of interest, the blat) gene can be used as a reporter of gene expression in mammalian cells. For more information about the bla(M) gene, see below.

A fluorescence resonance energy transfer (FRET)-enabled substrate, CCF2 to facilitate fluorescence detection of β-lactamase activity. In the absence or presence of β-lactamase reporter activity, cells loaded with the CCF2 substrate fluoresce green or blue, respectively. Comparing the ratio of blue to green fluorescence in a population of live cells or in a cell extract of your sample to a negative control provides a means to quantitate gene expression. For more information about the CCF2 substrate and how FRET works, refer to the GeneBLAzer™ Detection Kits manual.

β-Lactamase (bla) Gene

β-lactamase is the product encoded by the ampicillin resistance gene (b/a) and is the bacterial enzyme that hydrolyzes penicillins and cephalosporins. The bla gene is present in many cloning vectors and allows ampicillin selection in E. coli. β-lactamase enzyme activity is not found in mammalian cells.

bla(M) Gene

The GeneBLAzer™ Technology uses a modified bla gene as a reporter in mammalian cells. This bla gene is derived from the E. coli TEM-1 gene present in many cloning vectors (Zlokarnik, G., Negulescu, P. A., Knapp, T. E., Mere, L., Burres, N., Feng, L., Whitney, M., Roemer, K., and Tsien, R. Y. (1998). Quantitation of Transcription and Clonal Selection of Single Living Cells with b-Lactamase as Reporter. Science 279, 84-88), and has been modified in the following ways:

72 nucleotides encoding the first 24 amino acids of β-lactamase were deleted from the N-terminal region of the gene. These 24 amino acids comprise the bacterial periplasmic signal sequence, and deleting this region allows cytoplasmic expression of β-lactamase in mammalian cells.

The amino acid at position 24 was mutated from His to Asp to create an optimal Kozak sequence for optimal translation initiation.

This modified reporter gene is named bla(M).

Note: The TEM-1 gene also contains 2 mutations (at nucleotide positions 452 and 753) that distinguish it from the bla gene in pBR322 (Sutcliffe, J. G. (1978). Nucleotide Sequence of the Ampicillin Resistance Gene of Escherichia coli Plasmid pBR322. Proc. Nat. Acad. Sci. USA 75, 3737-3741).

Experimental Outline

The table below describes the general steps needed to clone and express your gene of interest.

TABLE 15
Experimental Outline

| Step | Action |
|---|---|
| 1 | Design PCR primers to clone your gene of interest in frame |
| | with the β-lactamase reporter gene. |
| 2 | Produce your blunt-end PCR product. |
| 3 | TOPO ® Clone your PCR product into a pcDNA6.2/GeneBLAzer- |
| | GW/D-TOPO ® vector and transform into One |
| | Shot ® Mach1 ™-T1^{R} E. coli. |
| | Select for transformants on LB agar plates containing 100 μg/ml |
| | ampicillin. |
| 4 | Analyze transformants for the presence and orientation of the |
| | insert by restriction digestion, PCR, or sequencing. |
| 5 | Prepare purified plasmid DNA for transfection. |
| 6 | Transfect your mammalian cell line with the |
| | pcDNA6.2/GeneBLAzer-GW/D-TOPO ® construct |
| | using your method of choice. Select for stable |
| | transfectants using Blasticidin, if desired. |
| 7 | Assay for β-lactamase reporter activity using the appropriate |
| | GeneBLAzer ™ Detection Kit. |

General Requirements for Designing PCR Primers

Designing Your PCR Primers

The design of the PCR primers to amplify your gene of interest is critical for expression. Consider the following when designing your PCR primers.

Sequences required to facilitate directional cloning;

Sequences required for proper translation initiation of your PCR product;

Sequences required to fuse your PCR product in frame with the β-lactamase reporter gene.

General Requirementsfor the Forward Primer

To enable directional cloning, the forward PCR primer must contain the sequence, CACC, at the 5′ end of the primer. The 4 nucleotides, CACC, base pair with the overhang sequence, GTGG, in each pcDNA6.2/GeneBLAzer-GW/D-TOPO® vector.

Example of Forward Primer Design

Below is the DNA sequence of the N-terminus of a theoretical protein and the proposed sequence for your forward PCR primer. The ATG initiation codon is underlined.

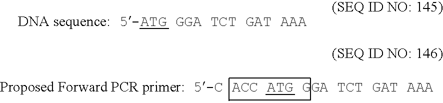

If you design the forward PCR primer as noted above, then the ATG initiation codon falls within the context of a Kozak sequence (see boxed sequence), allowing proper translation initiation of the PCR product in mammalian cells.

The first three base pairs of the PCR product following the 5′ CACC overhang will constitute a functional codon.

General Requirements for the Reverse Primer

In general, design the reverse PCR primer to allow you to clone your PCR product in frame with any C-terminal fusions, if desired. To ensure that your PCR product clones directionally with high efficiency, the reverse PCR primer MUST NOT be complementary to the overhang sequence GTGG at the 5′ end. A one base pair mismatch can reduce the directional cloning efficiency from 90% to 75%, and may increase the chances of your ORF cloning in the opposite orientation. We have not observed evidence of PCR products cloning in the opposite orientation from a two base pair mismatch, but this has not been tested thoroughly.

Example #1 of Reverse Primer Design

Below is the sequence of the C-terminus of a theoretical protein. You want to fuse the protein in frame with a C-terminal tag. The stop codon is underlined.

DNA sequence:

(SEQ ID NO: 147)

aag tcg gag cac tcg acg acG GTG tGA-3′

One possibility is to design the reverse PCR primer to start with the codon just up-stream of the stop codon, but the last two codons contain GTGG (underlined below), which is identical to the 4 bp overhang sequence. As a result, the reverse primer will be complementary to the 4 bp overhang sequence, increasing the probability that the PCR product will clone in the opposite orientation. You want to avoid this situation.

DNA sequence:

(SEQ ID NO:148)

aag tcg gag cac tcg acg acG GTG tGA-3′
Proposed Reverse PCR primer sequence:

(SEQ ID NO:149)

TG AGC TGG TGC GAG AAA-5′

Another possibility is to design the reverse primer so that it hybridizes just down-stream of the stop codon, but still includes the C-terminus of the ORF. Note that you will need to replace the stop codon with a codon for an innocuous amino acid such as glycine, alanine, or lysine (see below).

Example #2 of Reverse Primer Design

Below is the sequence for the C-terminus of a theoretical protein. The stop codon is underlined.

(SEQ ID NO:150)

. . . gcg gtt aag teg gag cac tcg acg act gca
tGA-3′

To fuse the ORF in frame with a C-terminal tag, remove the stop codon by starting with nucleotides homologous to the last codon (TGC) and continue upstream. The reverse primer will be:

(SEQ ID NO:151)

5′-TGC AGT CGT CGA GTG CTC CGA CTT-3′

This will amplify the C-terminus without the stop codon and allow you to join the ORF in frame with a C-terminal tag.

If you don't want to join the ORF in frame with a C-terminal tag, simply design the reverse primer to include the stop codon.

(SEQ ID NO:152)

5′-TCA TGC AGT CGT CGA GTG CTC CGA CTT-3′

Remember that the pcDNA6.2/GeneBLAzer-GW/D-TOPO® vectors accept blunt-end PCR products.

Do not add 5′ phosphates to your primers for PCR. This will prevent ligation into the pcDNA6.2/GeneBLAzer-GW/D-TOPO® vectors.

We recommend that you gel-purify your oligonucleotides, especially if they are long (>30 nucleotides).

Cloning into pcDNA 6.2/cGeneBLAzer-GW/D-TOPO®

Introduction

pcDNA6.2/cGeneBLAzer-GW/D-TOPO® allows expression of recombinant proteins containing a C-terminal β-lactamase reporter; however, you may use this vector to express native proteins or C-terminal fusion proteins. You may also use this vector in the Tag-On-Demand™ System.

Kozak Consensus Sequence

Your sequence of interest should contain a Kozak translation initiation sequence with an ATG initiation codon for proper initiation of translation (Kozak, M. (1987). An Analysis of 5′-Noncoding Sequences from 699 Vertebrate Messenger RNAs. Nucleic Acids Res. 15, 8125-8148; Kozak, M. (1991). An Analysis of Vertebrate mRNA Sequences: Intimations of Translational Control. J. Cell Biology 115, 887-903; Kozak, M. (1990). Downstream Secondary Structure Facilitates Recognition of Initiator Codons by Eukaryotic Ribosomes. Proc. Natl. Acad. Sci. USA 87, 8301-8305). An example of a Kozak consensus sequence is provided below. The ATG initiation codon is shown underlined.

(G/A)NNATGG

Other sequences are possible, but the G or A at position −3 and the G at position +4 are the most critical for function.

Additional Cloning Considerations

Consider the following when designing PCR primers to clone your DNA into pcDNA6.2/cGeneBLAzer-GW/D-TOPO®.

For all cases, design the forward PCR primer such that the ATG initiation codon is in the context of a Kozak consensus sequence (see above) and directly follows the 5′ CACC overhang. To design the reverse PCR primer, consider the following:

TABLE 16
Additional Cloning Considerations

| If you wish to . . . | Then . . . |
|---|---|
| include the | design the reverse PCR primer to remove the |
| β-lactamase reporter | native stop codon and preserve the reading frame |
| | through the bla(M) reporter gene |
| include the | design the reverse PCR primer to: |
| β-lactamase reporter | remove any native TAA or TGA stop codons |
| for use in the Tag-On- | include a TAG stop codon if one does not already |
| Demand ™ System | exist |
| | preserve the reading frame through the bla(M) |
| | reporter gene |
| not include the | design the reverse primer to include the native |
| β-lactamase reporter | sequence containing the stop codon or make sure |
| | the stop codon is upstream from the reverse PCR |
| | primer binding site |

TOPO® Cloning Site ofpcDNA 6.2/cGeneBLAzer-GW/D-TOPO®

Use FIG. 15 to help you design suitable PCR primers to clone your PCR product into pcDNA6.2/cGeneBLAzer-GW/D-TOPO®. The shaded region corresponds to sequences that will be transferred from the pcDNA6.2/cGeneBLAzer-GW/D-TOPO® vector into the entry clone following the BP recombination reaction.

Cloning into pcDNA 6.2/n GeneBLAzer-GW/D-TOPO®

Introduction

pcDNA6.2/nGeneBLAzer-GW/D-TOPO® allows expression of recombinant proteins containing an N-terminal β-lactamase reporter and a C-terminal V5 epitope tag, if desired, and contains an ATG initiation codon within the context of a Kozak consensus sequence. You may use this vector in the Tag-On-Demand System.

### Additional Cloning Considerations

TABLE 17
Additional Cloning Considerations

| If you wish to . . . | Then . . . |
|---|---|
| include the β-lactamase | design the forward primer to preserve the |
| reporter | reading frame with the bla(M) reporter gene |
| include the V5 epitope | design the reverse PCR primer to |
| tag | remove the native stop codon |
| | preserve the reading frame through the V5 |
| | epitope tag |
| include the V5 epitope | design the reverse PCR primer to |
| for use in the Tag-On- | remove any native TAA or TGA stop codons |
| Demand ™ System | include a TAG stop codon if one does not |
| | already exist |
| | preserve the reading frame through the V5 |
| | epitope tag |
| not include the V5 | design the reverse primer to include the native |
| epitope tag | sequence containing the stop codon or make |
| | sure the stop codon is upstream from the |
| | reverse PCR primer binding site |

TOPO® Cloning Site ofpcDNA 6.2/n GeneBLAzer-GW/D-TOPO®

Use FIG. 16 to help you design suitable PCR primers to clone your PCR product into pcDNA6.2/nGeneBLAzer-GW/D-TOPO®. The shaded region corresponds to sequences that will be transferred from the pcDNA6.2/nGeneBLAzer-GW/D-TOPO®vector into the entry clone following the BP recombination reaction.

Producing Blunt-End PCR Products

Introduction

Once you have decided on a PCR strategy and have synthesized the primers, you are ready to produce your blunt-end PCR product using any thermostable, proofreading polymerase. Follow the guidelines below to produce your blunt-end PCR product.

Materials Needed

You should have the following materials on hand before beginning.

Note: dNTPs (adjusted to pH 8) are provided in the kit.

Thermocycler and thermostable, proofreading polymerase

10×PCR buffer appropriate for your polymerase

DNA template and primers for PCR product

Producing PCR Products

Set up a 25 μl or 50 μl PCR reaction using the guidelines below:

Follow the instructions and recommendations provided by the manufacturer of your thermostable, proofreading polymerase to produce blunt-end PCR products.

Use the cycling parameters suitable for your primers and template. Make sure to optimize PCR conditions to produce a single, discrete PCR product.

Use a 7 to 30 minute final extension to ensure that all PCR products are completely extended.

After cycling, place the tube on ice or store at −20° C. for up to 2 weeks. Proceed to Checking the PCR Product, below.

Checking the PCR Product

After you have produced your blunt-end PCR product, use agarose gel electrophoresis to verify the quality and quantity of your PCR product. Check for the following outcomes below.

Be sure you have a single, discrete band of the correct size. If you do not have a single, discrete band, follow the manufacturer's recommendations for optimizing your PCR with the polymerase of your choice. Alternatively, you may gel-purify the desired product.

Estimate the concentration of your PCR product. You will use this information when setting up your TOPOS Cloning reaction.

Performing the TOPO® Cloning Reaction

Introduction

Once you have produced the desired PCR product, you are ready to TOPO® Clone it into a pcDNA6.2/GeneBLAzer-GW/D-TOPO® vector and transform the recombinant vector into Mach1™-T1® cells.

Amount of PCR Product to Use in the TOPO® Cloning Reaction

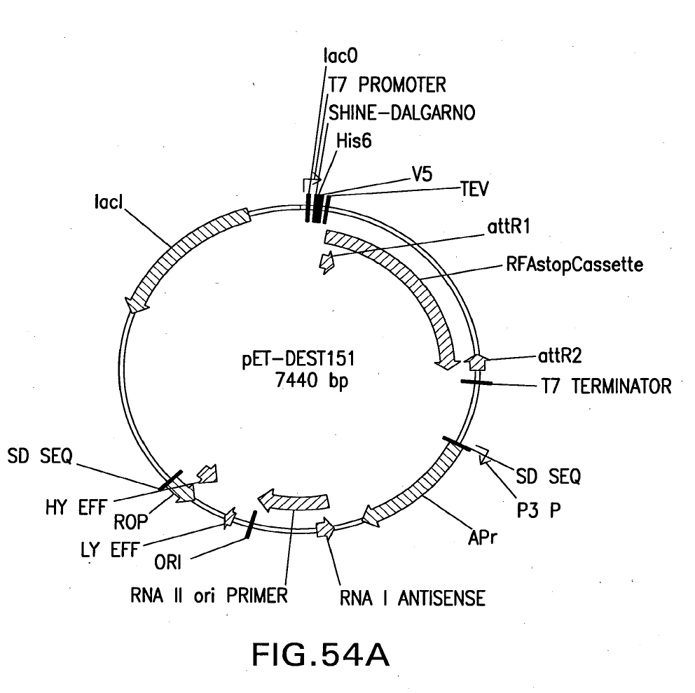

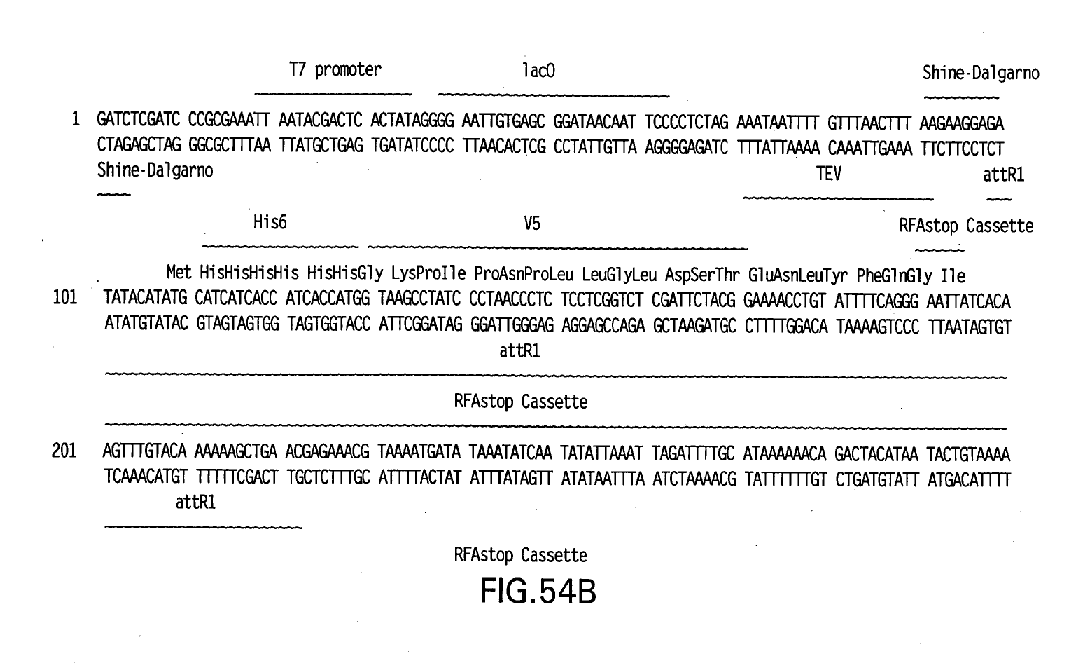

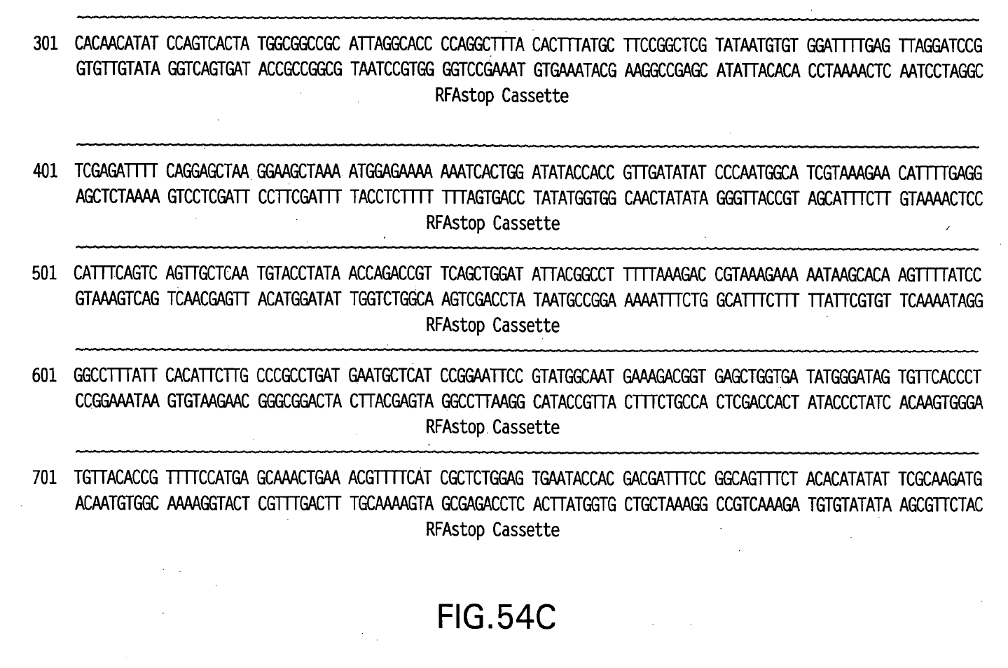

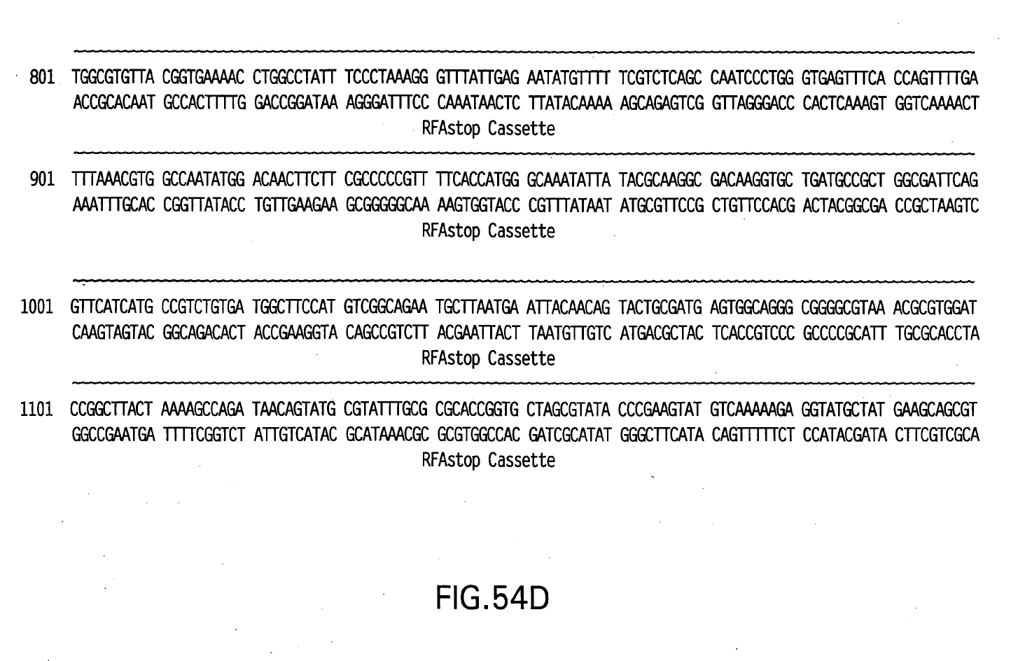

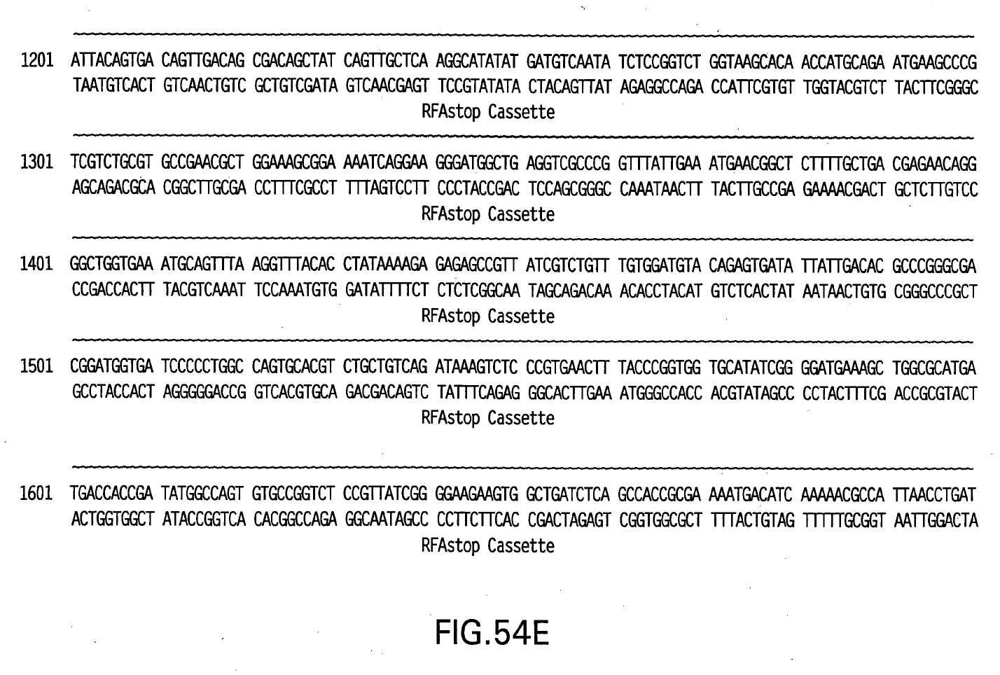

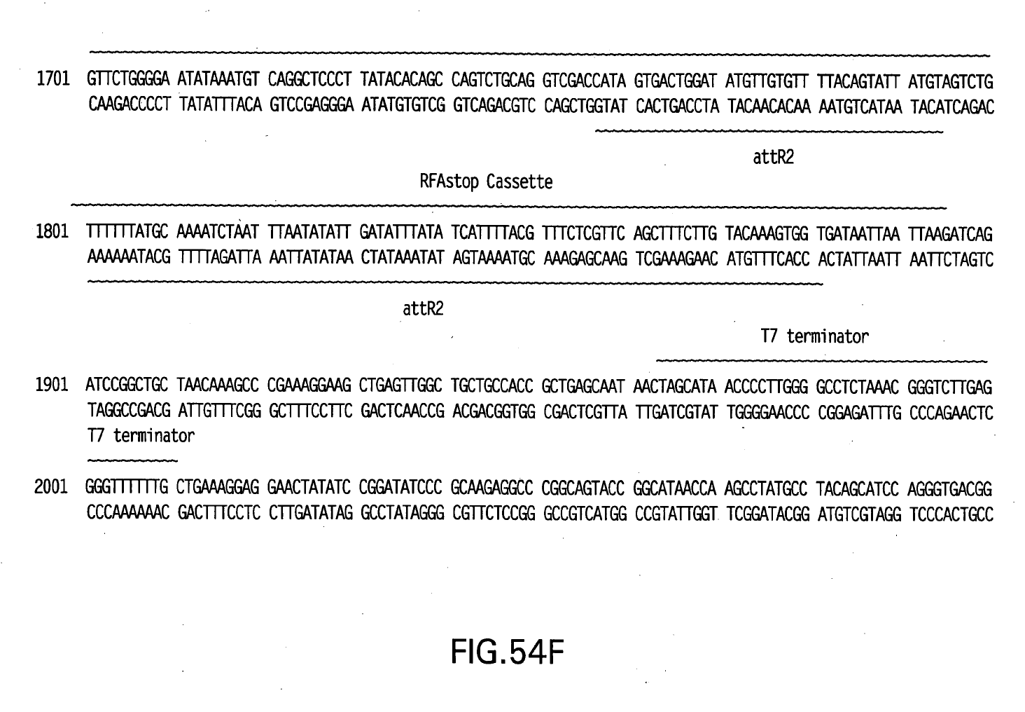

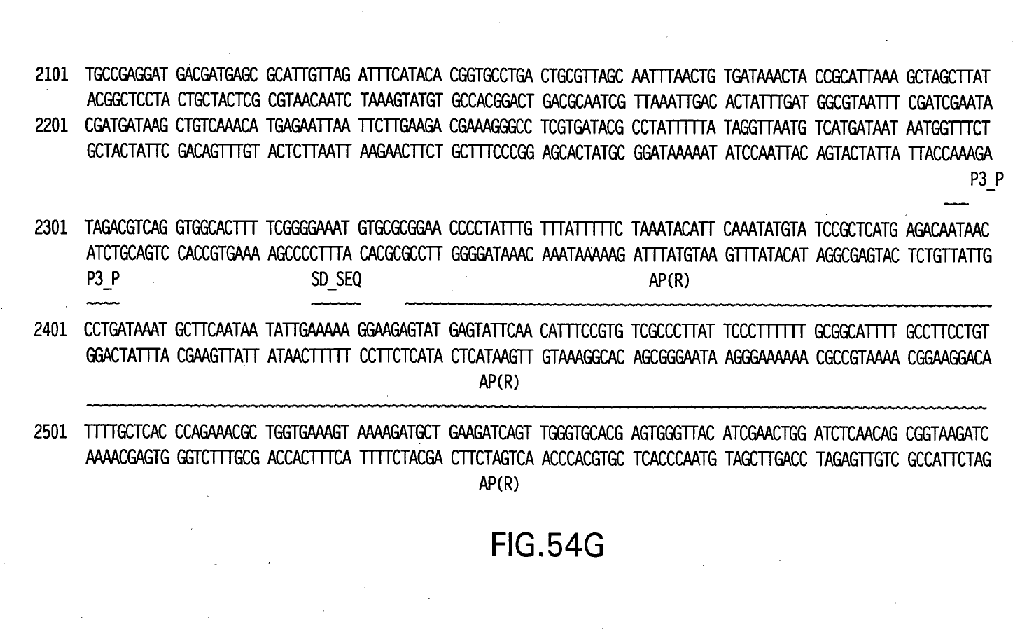

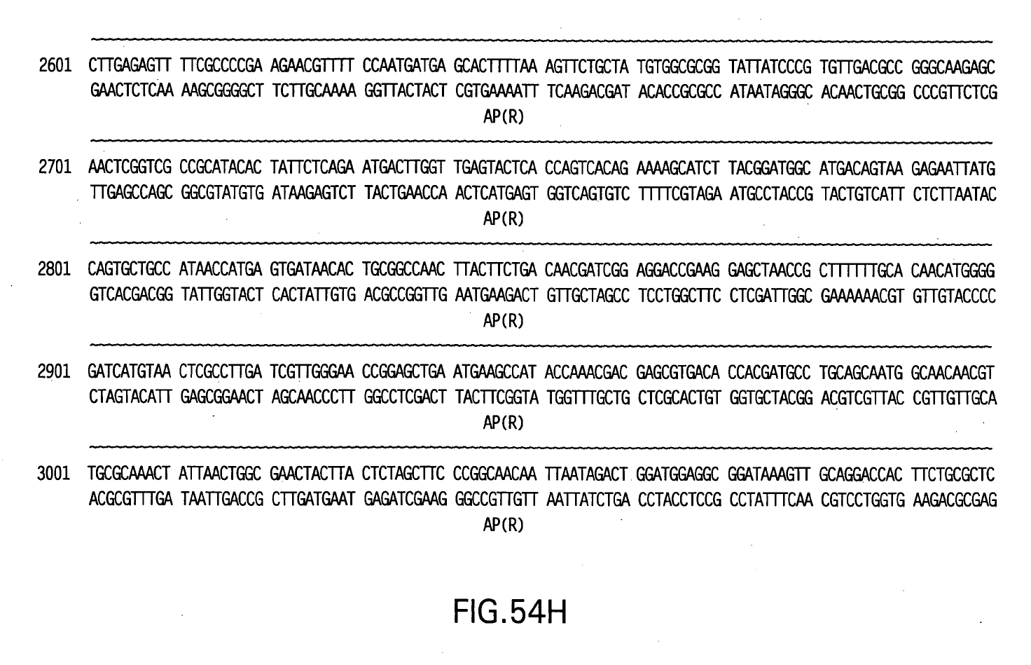

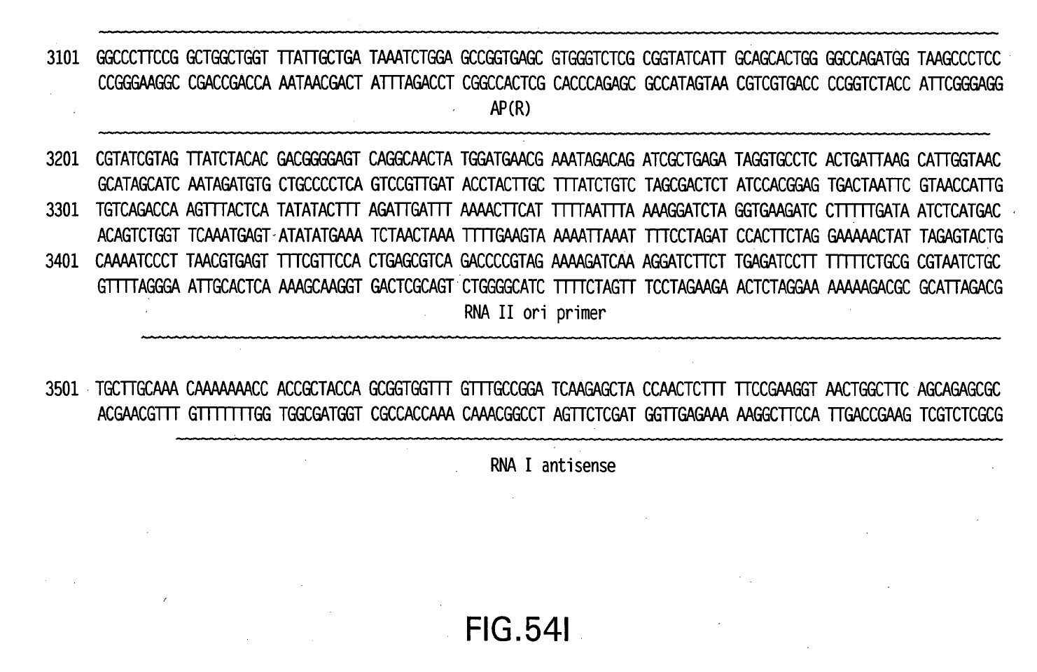

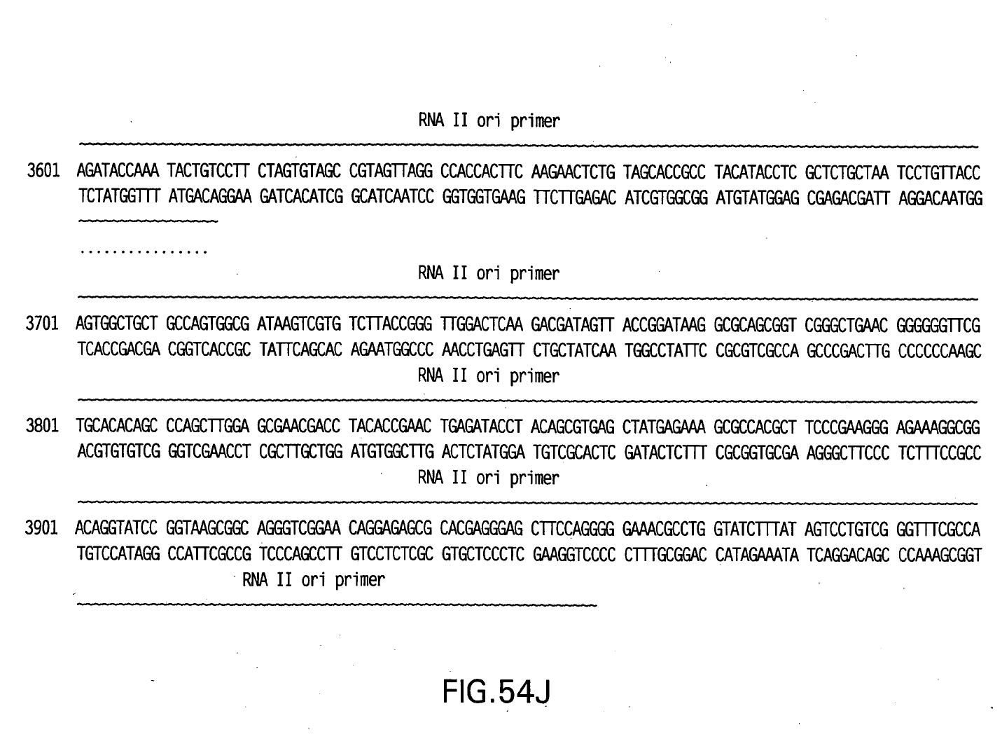

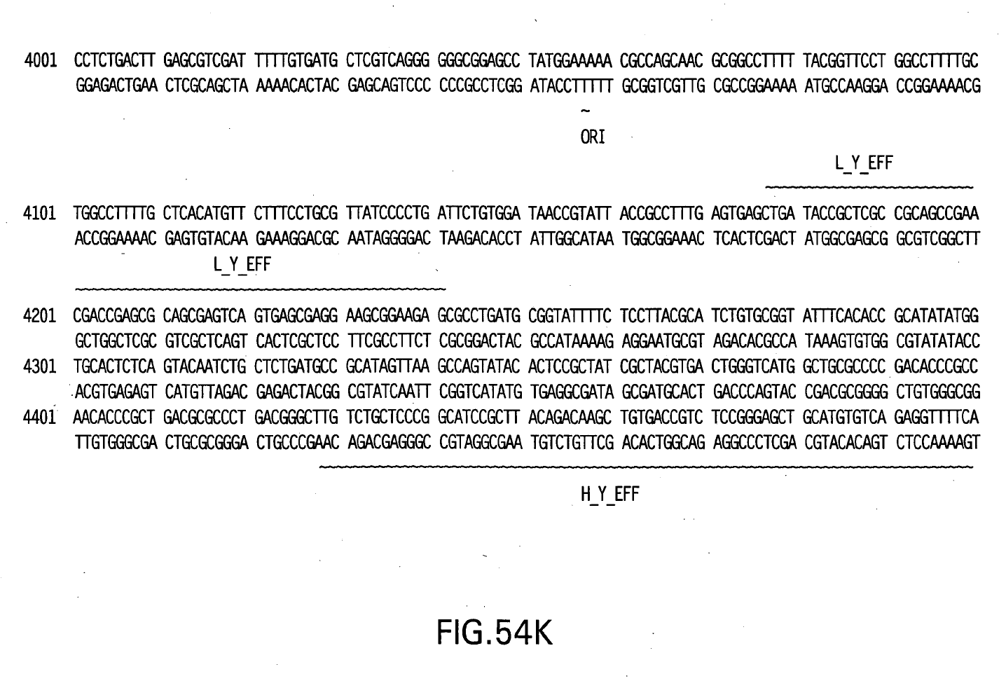

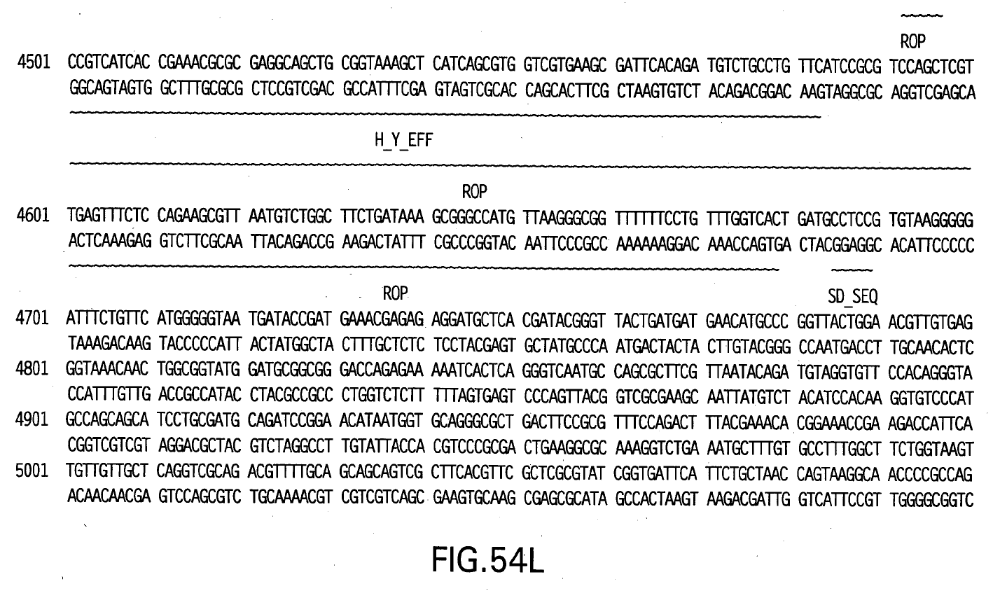

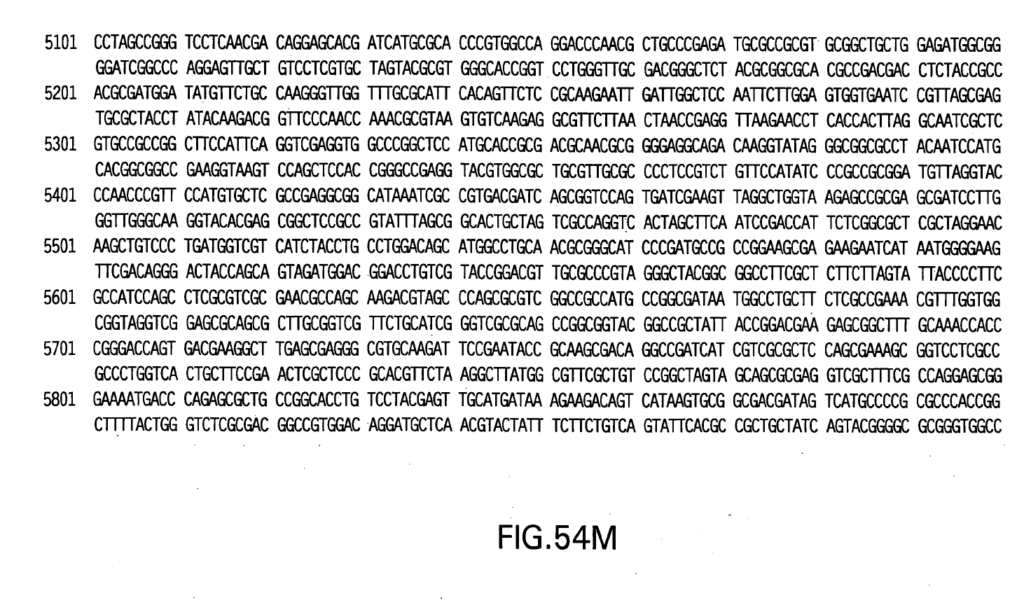

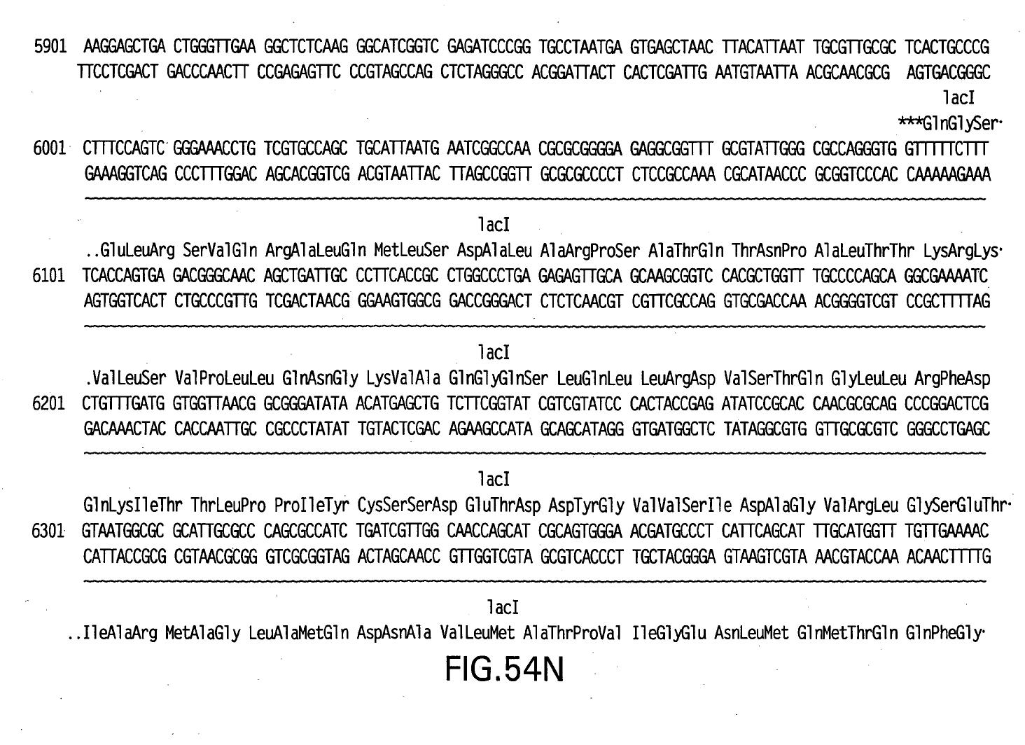

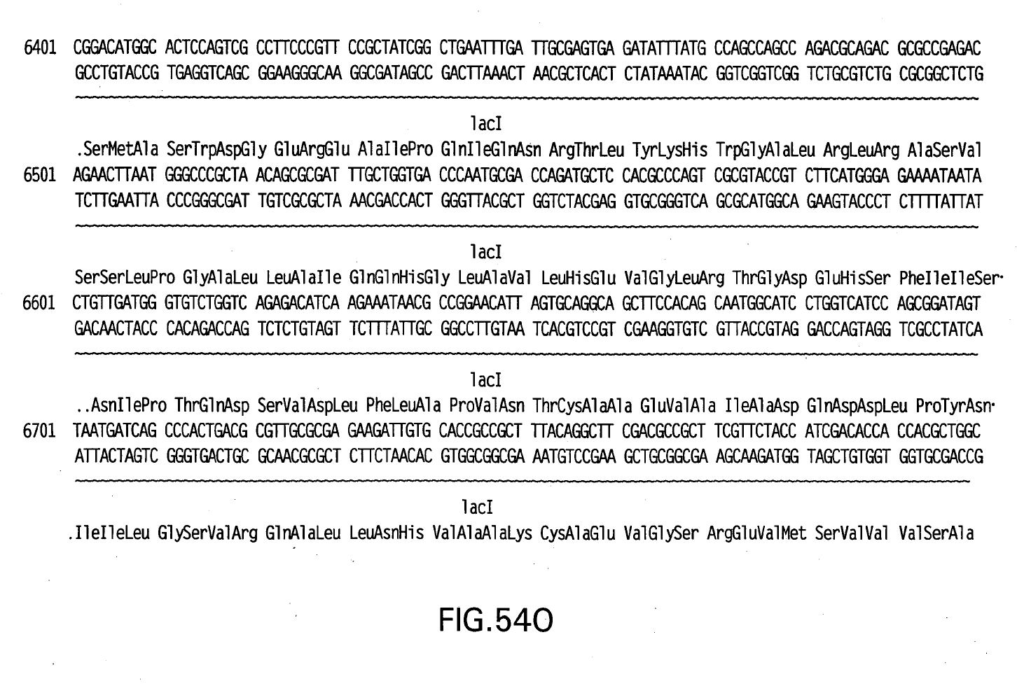

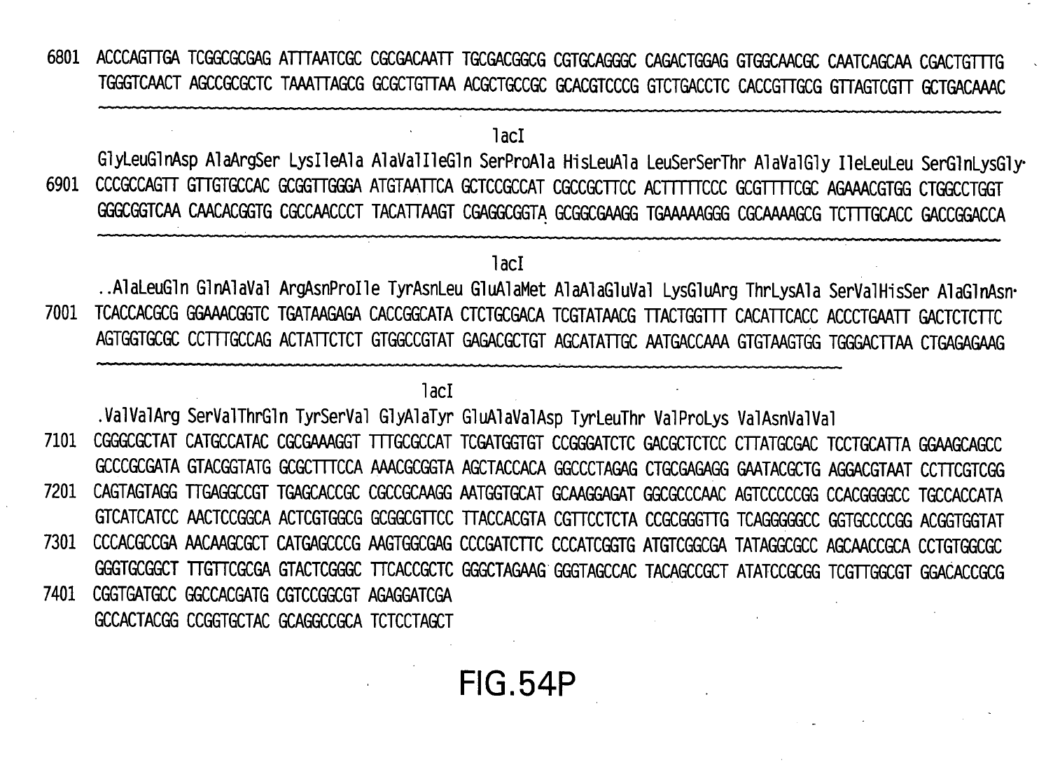

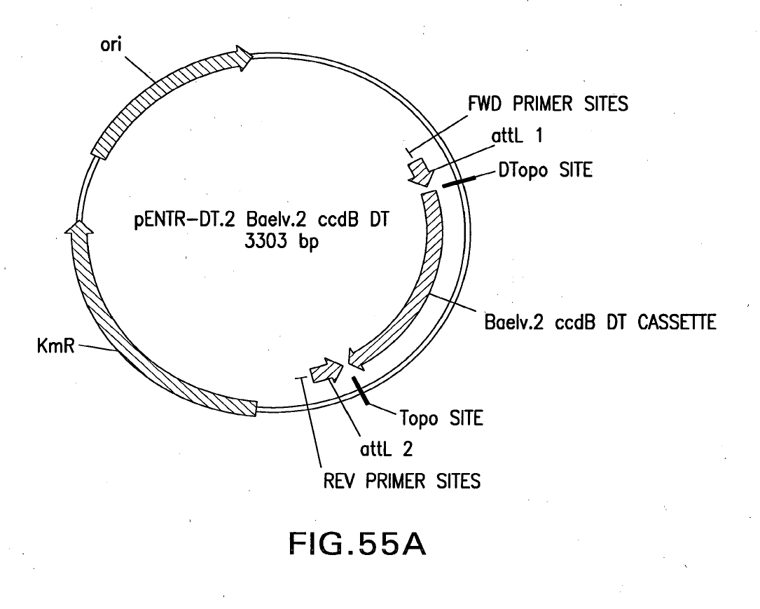

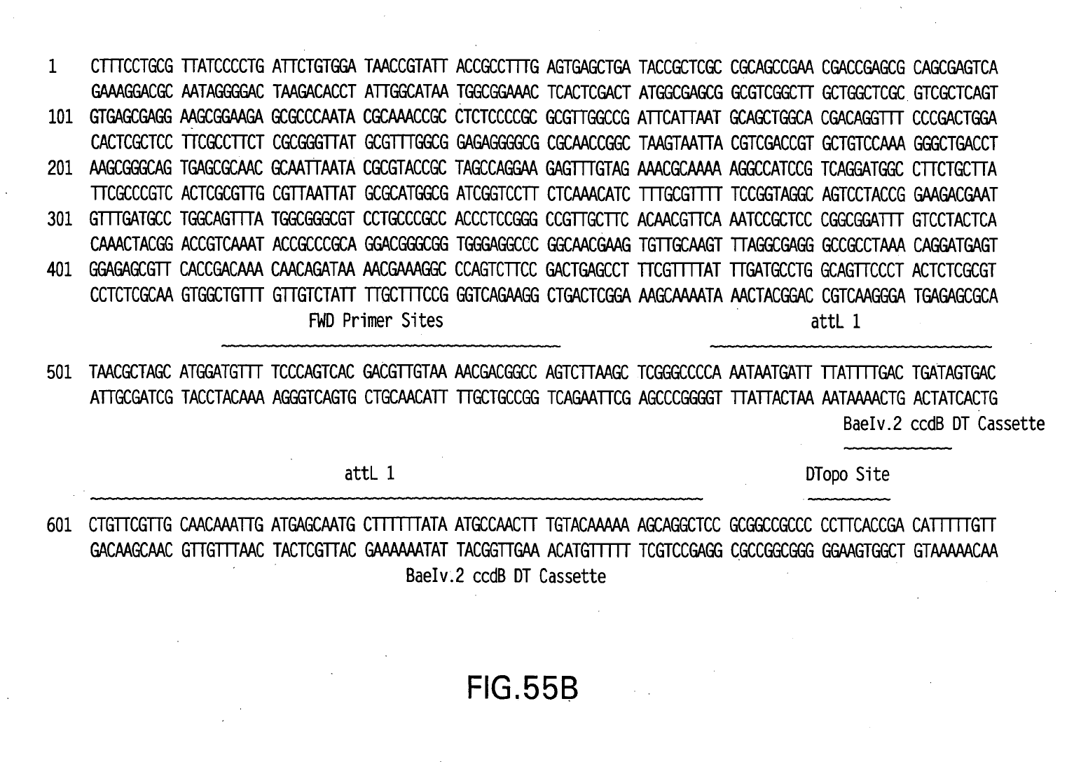

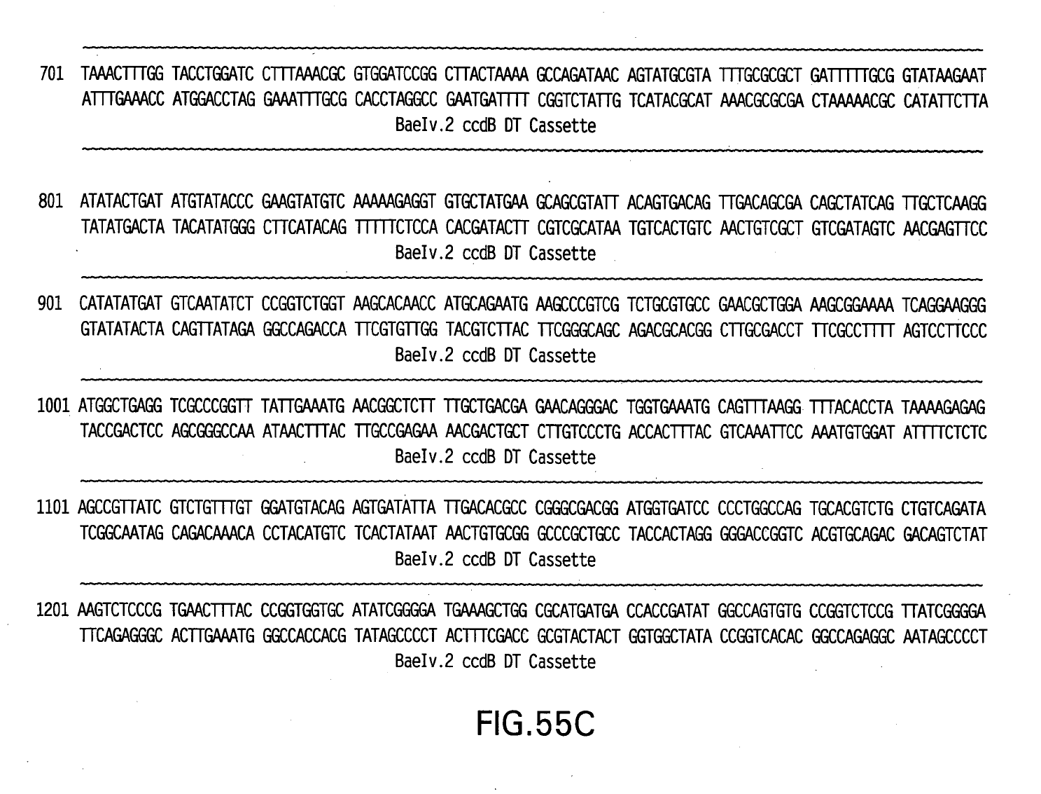

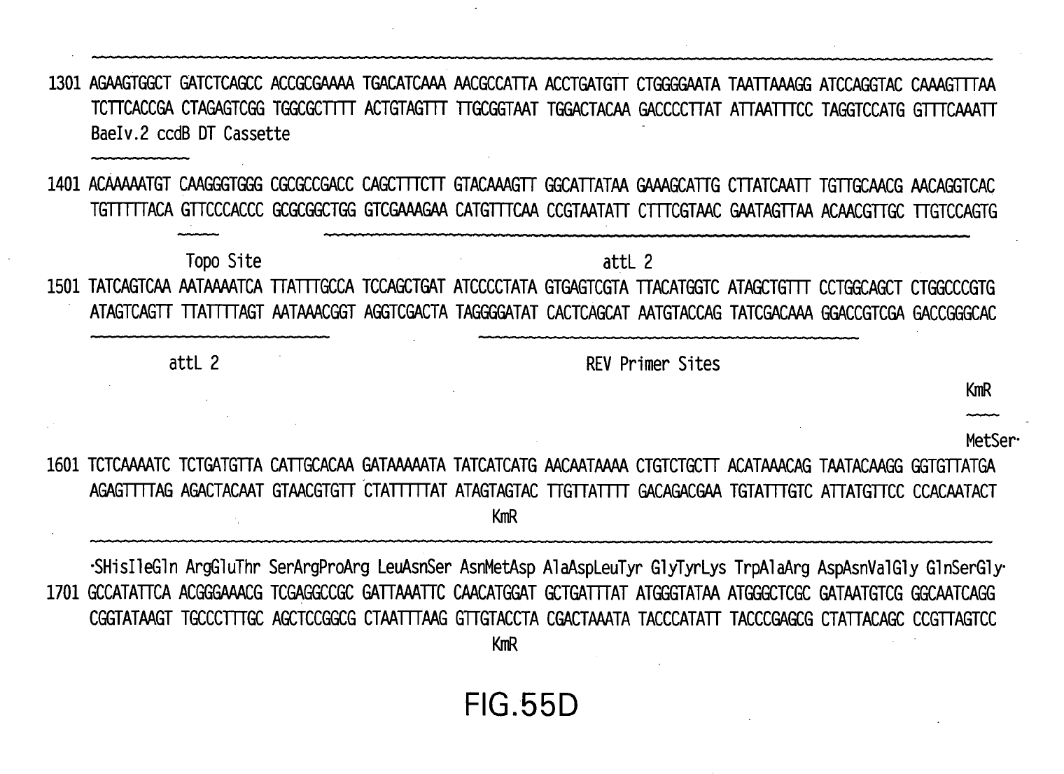

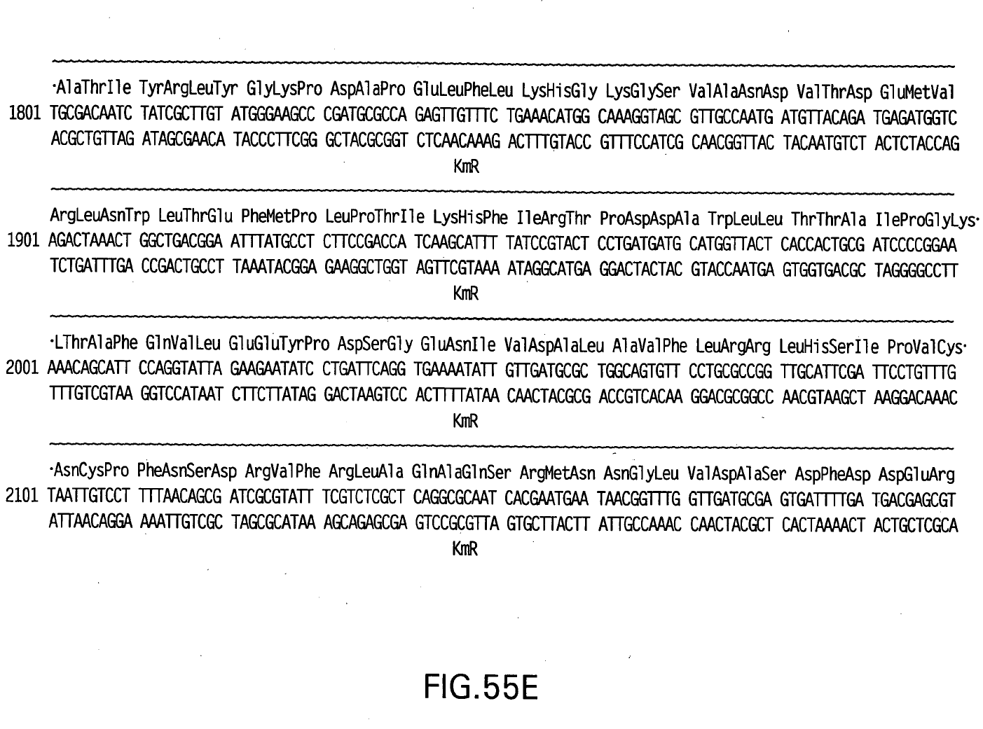

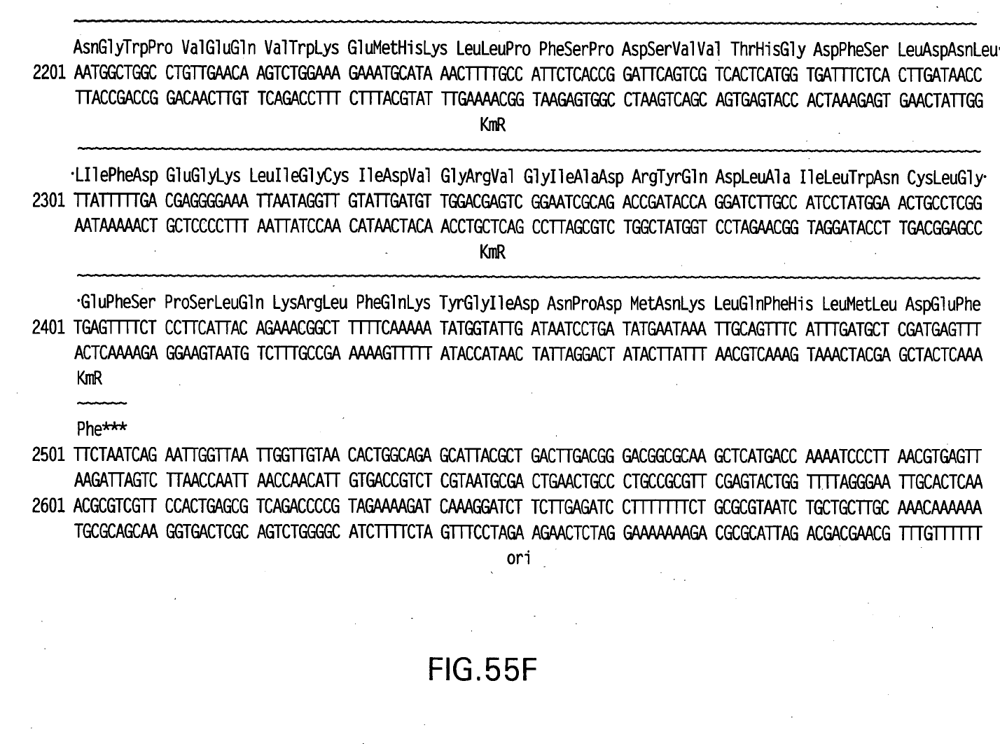

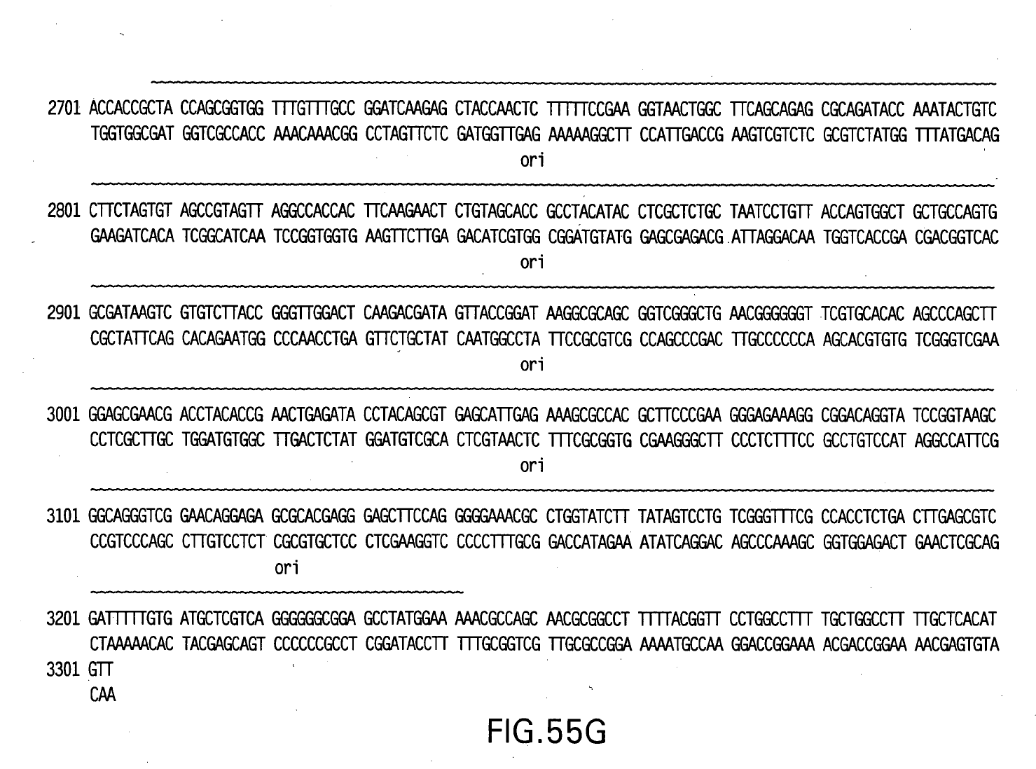

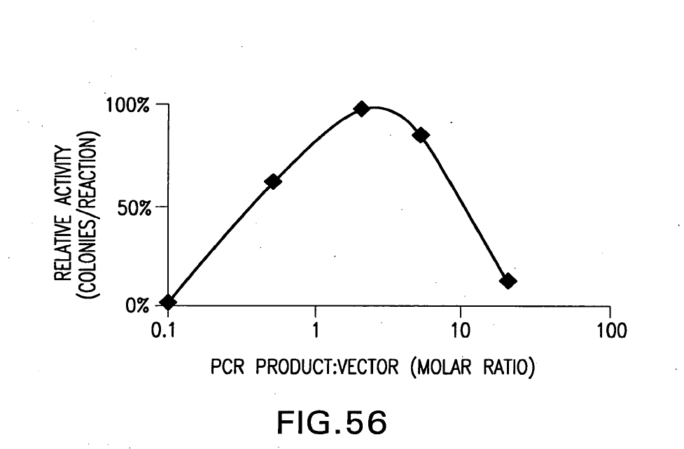

When performing directional TOPO® Cloning, we have found that the molar ratio of PCR product:TOPO® vector used in the reaction is critical to its success. To obtain the highest TOPO® Cloning efficiency, use a 0.5:1 to 2:1 molar ratio of PCR product:TOPO®vector (see FIG. 54). Note that the TOPO® Cloning efficiency decreases significantly if the ratio of PCR product: TOPO® vector is <0.1:1 or >5:1. These results are generally obtained if too little PCR product is used (i.e. PCR product is too dilute) or if too much PCR product is used in the TOPO® Cloning reaction. If you have quantitated the yield of your PCR product, you may need to adjust the concentration of your PCR product before proceeding to TOPO® Cloning.

Tip: For the pcDNA6.2/GeneBLAzer-GW/D-TOPO® vectors, using 1-5 ng of a 1 kb PCR product or 5-10 ng of a 2 kb PCR product in a TOPO® Cloning reaction generally results in a suitable number of colonies.

Using Salt Solution in the TOPO® Cloning Reaction

You will perform TOPO® Cloning in a reaction buffer containing salt (i.e. using the stock salt solution provided in the kit). Note that the amount of salt added to the TOPO® Cloning reaction varies depending on whether you plan to transform chemically competent cells (provided) or electrocompetent cells.

If you are transforming chemically competent E. coli, use the stock Salt Solution as supplied and set up the TOPO® Cloning reaction as directed below.

If you are transforming electrocompetent E. coli, the amount of salt in the TOPO® Cloning reaction must be reduced to 50 mM NaCl, 2.5 mM MgCl₂ to prevent arcing during electroporation. Dilute the stock Salt Solution 4-fold with water to prepare a 300 mM NaCl, 15 mM MgCl₂ Dilute Salt Solution. Use the Dilute Salt Solution to set up the TOPO® Cloning reaction as directed below.

Performing the TOPO® Cloning Reaction

Use the procedure below to perform the TOPO® Cloning reaction. Set up the TOPO® Cloning reaction depending on whether you plan to transform chemically competent E. coli or electrocompetent E. coli. Reminder: For optimal results, be sure to use a 0.5:1 to 2:1 molar ratio of PCR product:TOPO® vector in your TOPO® Cloning reaction.

Note: The blue color of the TOPO® vector solution is normal and is used to visualize the solution.

TABLE 18
TOPO Cloning Procedure

| | Chemically Competent | Electrocompetent |
|---|---|---|
| Reagents* | E. coli | E. coli |
| Fresh PCR Product | 0.5 to 4 μl | 0.5 to 4 μl |
| Salt Solution | 1 μl | — |
| Dilute Salt Solution (1:4) | — | 1 μl |
| Sterile Water | add to a final volume | add to a final volume |
| | of 5 μl | of 5 μl |
| pcDNA6.2/GeneBLAzer- | 1 μl | 1 μl |
| GW/D-TOPO ® Vector | | |
| Final Volume | 6 μl | 6 μl |

*Store all reagents at −20° C. when finished. Salt solution and water can be stored at room temperature or +4° C.

Mix reaction gently and incubate for 5 minutes at room temperature (22-23° C.).

Note: For most applications, 5 minutes will yield a sufficient number of colonies for analysis. Depending on your needs, the length of the TOPO® Cloning reaction can be varied from 30 seconds to 30 minutes. For routine subcloning of PCR products, 30 seconds may be sufficient. For large PCR products (>1 kb) or if you are TOPO® Cloning a pool of PCR products, increasing the reaction time may yield more colonies.

Place the reaction on ice and proceed to Transforming One Shot® Mach1™-TI® Competent Cells.

Note: You may store the TOPO® Cloning reaction at −20° C. overnight.

Transforming One Shot® Mach1™-T1® Competent Cells

Introduction

Once you have performed the TOPO® Cloning reaction, you will transform your GeneBLAzer™ Directional TOPO® construct into competent E. coli. One Shot® Mach1™-T1® Chemically Competent E. coli (Box 2) are included to facilitate transformation, however, you may also transform other chemically competent cells (e.g. TOP10) or electrocompetent cells. Protocols to transform chemically competent or electrocompetent E. coli are provided in this section.

Blasticidin Selection

The presence of the EM7 promoter and the Blasticidin resistance gene in the pcDNA6.2/GeneBLAzer-GW/-D-TOPO® vectors allows for selection of E. coli transformants using Blasticidin. For selection, use Low Salt LB agar plates containing 100 μg/ml Blasticidin. For Blasticidin to be active, the salt concentration of the medium must remain low (<90 mM) and the pH must be 7.0.

Blasticidin is available separately from Invitrogen.

The Mach1™-T1® strain allows you to visualize colonies 8 hours after plating on ampicillin selective plates. If you are using Blasticidin selection, you will need to incubate plates overnight in order to visualize colonies.

With the Mach1™-T1® strain, you may also prepare plasmid DNA 4 hours after inoculating a single, overnight-grown colony. Note that you will get sufficient growth of transformed cells within 4 hours with either ampicillin or Blasticidin selection.

Materials Needed

You should have the following materials on hand before beginning:

TOPO® Cloning reaction from Performing the TOPO® Cloning Reaction, Step 2

S.O.C. medium (included with the kit)

42° C. water bath (or electroporator with cuvettes, optional)

LB plates containing 100 pg/ml ampicillin or Low Salt LB plates containing 100 μg/ml Blasticidin (two for each transformation)

37° C. shaking and non-shaking incubator

There is no blue-white screening for the presence of inserts. Most transformants will contain recombinant plasmids with the PCR product of interest cloned in the correct orientation. Sequencing primers are included in the kit to sequence across an insert in the multiple cloning site to confirm orientation and reading frame.

### Preparing for Transformation

For each transformation, you will need one vial of competent cells and two selective plates.

Equilibrate a water bath to 42° C. (for chemical transformation) or set up your electroporator if you are using electrocompetent E. coli.

Warm the vial of S.O.C. medium from Box 2 to room temperature.

Warm selective plates at 37° C. for 30 minutes.

Thaw on ice 1 vial of One Shot® Mach1™-T1® cells from Box 2 for each transformation.

If you are using ampicillin selection and wish to visualize colonies within 8 hours of plating, it is essential that you prewarm your LB plates containing 100 μg/ml ampicillin prior to spreading.

One Shot® Mach1™-T1® Chemical Transformation Protocol

Add 2 μl of the TOPO® Cloning reaction from Performing the TOPO® Cloning Reaction into a vial of One Shot® Mach1™-T1® Chemically Competent E. coli and mix gently. Do not mix by pipetting up and down.

Incubate on ice for 5 to 30 minutes.

Note: Longer incubations on ice seem to have a minimal effect on transformation efficiency. The length of the incubation is at the user's discretion.

Heat-shock the cells for 30 seconds at 42° C. without shaking.

Immediately transfer the tubes to ice.

Add 250 μl of room temperature S.O.C. medium.

Cap the tube tightly and shake the tube horizontally (200 rpm) at 37° C. for 1 hour.

Spread 50-200 μl from each transformation on a prewarmed selective plate. We recommend plating two different volumes to ensure that at least one plate will have well-spaced colonies.

Incubate plates at 37° C. If you are using ampicillin selection, visible colonies should appear within 8 hours. For Blasticidin selection, incubate plates overnight.

An efficient TOPO® Cloning reaction should produce several hundred colonies. Pick ˜5 colonies for analysis.

Transformation by Electroporation

Use ONLY electrocompetent cells for electroporation to avoid arcing. Do not use the One Shot® Mach1-T1® chemically competent cells for electroporation.

Add 2 μl of the TOPO® Cloning reaction from Performing the TOPO® Cloning Reaction into a sterile microcentrifuge tube containing 50 μl of electrocompetent E. coli and mix gently. Do not mix by pipetting up and down. Avoid formation of bubbles. Transfer the cells to a 0.1 cm cuvette.

Electroporate your samples using your own protocol and your electroporator.

Note: If you have problems with arcing, see below.

Immediately add 250 μl of room temperature S.O.C. medium.

Transfer the solution to a 15 ml snap-cap tube (e.g. Falcon) and shake for at least 1 hour at 37° C. to allow expression of the ampicillin resistance gene.

Spread 20-100 μl from each transformation on a prewanned selective plate and incubate overnight at 37° C. To ensure even spreading of small volumes, add 20 μl of S.O.C. medium. We recommend that you plate two different volumes to ensure that at least one plate will have well-spaced colonies.

An efficient TOPO® Cloning reaction may produce several hundred colonies. Pick ˜5 colonies for analysis.

To prevent arcing of your samples during electroporation, the volume of cells should be between 50 and 80 μl (0.1 cm cuvettes) or 100 to 200 μl (0.2 cm cuvettes).

If you experience arcing during transformation, try one of the following suggestions:

Reduce the voltage normally used to charge your electroporator by 10%

Reduce the pulse length by reducing the load resistance to 100 ohms

Ethanol precipitate the TOPO® Cloning reaction and resuspend in water prior to electroporation

Analyzing Transformants

Analyzing Positive Clones

Pick 5 colonies and culture them overnight in LB or SOB medium containing 50-100 μg/ml ampicillin.

2. Isolate plasmid DNA using your method of choice. If you need ultra-pure plasmid DNA for automated or manual sequencing, we recommend using the PureLink HQ Mini Plasmid Purification Kit (Catalog no. K2100-01).

3. Analyze the plasmids by restriction analysis to confirm the presence and correct orientation of the insert. Use a restriction enzyme or a combination of enzymes that cut once in the vector and once in the insert.

Sequencing Primers for pcDNA 6.2/cGeneBLAzer-GW/D-TOPO®

To confirm that your gene of interest is in frame with the bla(M) reporter gene, you may sequence your construct, if desired. Keep the following in mind when designing your sequencing primers:

Use a forward primer which hybridizes within the 3′ end of your gene of interest to sequence through the 5′ region of the bla(M) reporter gene.

Do not use a reverse primer that hybridizes within the bla(M) reporter gene. Any primer that hybridizes within the bla(M) reporter gene will also hybridize within the ampicillin resistance gene, contaminating your results.

Note: Because you will not be using a reverse primer, you will only be able to sequence the sense strand of your construct.

Use the T7 Promoter primer (supplied with Catalog nos. 12578-076 and 12578-084) to sequence through the 5′ region of your gene of interest.

Sequencing Primers for pcDNA 6.2/n GeneBLAzer-GW/D-TOPO®

To confirm that your gene of interest is in frame with the bla(M) reporter gene or the V5 epitope tag, you may sequence your construct, if desired. Keep the following in mind when designing your sequencing primers:

Use a reverse primer which hybridizes within the 5′ end of your gene of interest to sequence through the 3′ region of the bla(M) reporter gene.

Do not use a forward primer that hybridizes within the bla(M) reporter gene. Any primer that hybridizes within the β-lactamase reporter gene will also hybridize within the ampicillin resistance gene, contaminating your results.

Note: Because you will not be using a forward primer, you will only be able to sequence the anti-sense strand of your construct.

Use the TK polyA Reverse primer (supplied with Catalog nos. 12578-092 and 12578-100) to sequence through the V5 epitope.

If you download the sequence for pcDNA6.2/cGeneBLAzer-GW/D-TOPO® or pcDNA6.2/cGeneBLAzer-GW/D-TOPO® from our Web site, note that the overhang sequence (GTGG) will be shown already hybridized to CACC. No DNA sequence analysis program allows us to show the overhang without the complementary sequence.

Analyzing Transformants by PCR

You may analyze positive transformants using PCR. If you are using pcDNA6.2/cGeneBLAzer-GW/D-TOPO®, use a combination of the T7 Promoter primer and a primer that hybridizes within your insert. If you are using pcDNA6.2/nGeneBLAzer-GW/D-TOPO®, use a combination of the TK polyA Reverse primer and a primer that hybridizes within your insert.

You will have to determine the amplification conditions. If you are using this technique for the first time, we recommend performing restriction analysis in parallel. Artifacts may be obtained because of mispriming or contaminating template. The protocol below is provided for your convenience. Other protocols are suitable.

Materials Needed

PCR SuperMix High Fidelity (Invitrogen, Catalog no. 10790-020) Appropriate forward and reverse PCR primers (20 μM each)

Procedure

1. For each sample, aliquot 48 μl of PCR SuperMix High Fidelity into a 0.5 ml microcentrifuge tube. Add 1 μl each of the forward and reverse PCR primer.

2. Pick 5 colonies and resuspend them individually in 50 μl of the PCR cocktail from Step 1, above.

3. Incubate reaction for 10 minutes at 94° C. to lyse cells and inactivate nucleases.

4. Amplify for 20 to 30 cycles.

5. For the final extension, incubate at 72° C. for 10 minutes. Store at +4° C.

6. Visualize by agarose gel electrophoresis.

Long-Term Storage

Once you have identified the correct clone, be sure to purify the colony and make a glycerol stock for long-term storage. We recommend that you store a stock of plasmid DNA at −20° C.

Streak the original colony out for single colony on LB plates containing 50-100 μg/ml ampicillin.

Isolate a single colony and inoculate into 1-2 ml of LB containing 50-100 μg/ml ampicillin.

Grow until culture reaches stationary phase.

Mix 0.85 ml of culture with 0.15 ml of sterile glycerol and transfer to a cryovial.

Store at −80° C.

Transfecting Cells

Introduction

This section provides general information to transfect your expression clone into the mammalian cell line of choice. We recommend that you include a positive control vector (pcDNA™6.2/cGeneBLAzer™-GW/lacZ or pcDNA™6.2/nGeneBLAzer™-GW/lacZ) and a mock transfection (negative control) in your experiments to evaluate your results.

If you plan to detect β-lactamase reporter activity in vivo using the GeneBLAzer In Vivo Detection Kit (supplied with Catalog nos. 12578-084 and 12578-100 only), note that a number of factors including cell type and cell density can influence the degree of the fluorescence signal detected. We recommend taking these factors into account when designing your transfection experiment.

Plasmid Preparation

Once you have generated your expression clone, you must isolate plasmid DNA for transfection. Plasmid DNA for transfection into eukaryotic cells must be very clean and free from phenol and sodium chloride. Contaminants will kill the cells, and salt will interfere with lipid complexing, decreasing transfection efficiency. We recommend isolating plasmid DNA using the PureLink HQ Mini Plasmid Purification Kit (Catalog no. K2100-01) or CsCl gradient centrifugation.

Positive Control

pcDNA™6.2/cGeneBLAzer™-GW/lacZ or pcDNA™6.2/nGeneBLAzer™-GW/lacZ is provided as a positive control vector for mammalian cell transfection and expression and may be used to optimize recombinant protein expression levels in your cell line. These vectors allow expression of the β-galactosidase gene with either an N-terminal or C-terminal fusion to the β-lactamase reporter.

To propagate and maintain the plasmid:|

Use the stock solution to transform a recA, endA E. coli strain like Mach1™, TOP10, DH5α™, or equivalent.

Select transformants on LB agar plates containing 50-100 μg/ml ampicillin.

Prepare a glycerol stock of a transformant containing plasmid for long-term storage.

Methods of Transfection

For established cell lines (e.g. HeLa), consult original references or the supplier of your cell line for the optimal method of transfection. We recommend that you follow exactly the protocol for your cell line. Pay particular attention to medium requirements, when to pass the cells, and at what dilution to split the cells. Further information is provided in Current Protocols in Molecular Biology (Ausubel, F. M., Brent, R., Kingston, R. E., Moore, D. D., Seidman, J. G., Smith, J. A., and Struhl, K. (1994). Current Protocols in Molecular Biology (New York: Greene Publishing Associates and Wiley-Interscience)).

Methods for transfection include calcium phosphate (Chen, C., and Okayama, H. (1987). High-Efficiency Transformation of Mammalian Cells by Plasmid DNA. Mol. Cell. Biol. 7, 2745-2752; Wigler, M., Silverstein, S., Lee, L.-S., Pellicer, A., Cheng, Y.-C., and Axel, R. (1977). Transfer of Purified Herpes Virus Thymidine Kinase Gene to Cultured Mouse Cells. Cell 11, 223-232), lipid-mediated (Felgner, P. L., Holm, M., and Chan, H. (1989). Cationic Liposome Mediated Transfection. Proc. West. Pharmacol. Soc. 32, 115-121; Felgner, P. L. a., and Ringold, G. M. (1989). Cationic Liposome-Mediated Transfection. Nature 337, 387-388) and electroporation (Chu, G., Hayakawa, H., and Berg, P. (1987). Electroporation for the Efficient Transfection of Mammalian Cells with DNA. Nucleic Acids Res. 15, 1311-1326; Shigekawa, K., and Dower, W. J. (1988). Electroporation of Eukaryotes and Prokaryotes: A General Approach to the Introduction of Macromolecules into Cells. BioTechniques 6, 742-751). For high efficiency transfection in a broad range of mammalian cell lines, we recommend using Lipofectamine™ 2000 Reagent (Catalog no. 11668-027) available from Invitrogen.

Creating Stable Cell Lines

Introduction

The GeneBLAzer™ Directional TOPO™ vectors contain the Blasticidin resistance gene to allow selection of stable cell lines. If you wish to create stable cell lines, transfect your construct into the mammalian cell line of choice and select for foci using Blasticidin. General information and guidelines are provided below.

To obtain stable transfectants, we recommend that you linearize your pcDNA6.2/GeneBLAzer-GW/D-TOPO® construct before transfection. While linearizing the vector may not improve the efficiency of transfection, it increases the chances that the vector does not integrate in a way that disrupts elements necessary for expression in mammalian cells. To linearize your construct, cut at a unique site that is not located within a critical element or within your gene of interest.

### Determining Blasticidin Sensitivity

To successfully generate a stable cell line expressing your protein of interest, you need to determine the minimum concentration of Blasticidin required to kill your untransfected host cell line by performing a kill curve experiment (see below). Typically, concentrations ranging from 2.5 to 10 μg/ml Blasticidin are sufficient to kill most untransfected mammalian cell lines. Blasticidin is available separately from Invitrogen (Catalog no. R210-01).

Plate cells at approximately 25% confluence. Prepare a set of 6 plates.

On the following day, replace the growth medium with fresh growth medium containing varying concentrations of Blasticidin (e.g. 0, 1, 3, 5, 7.5, and 10 μg/ml Blasticidin).

Replenish the selective media every 3-4 days, and observe the percentage of surviving cells.

Count the number of viable cells at regular intervals to determine the appropriate concentration of Blasticidin that prevents growth within 10-14 days after addition of Blasticidin.

### Generating Stable Cell Lines

Once you have determined the appropriate Blasticidin concentration to use for selection, you can generate a stable cell line expressing your pcDNA6.2/GeneBLAzer-GW/D-TOPO® construct.

Transfect the mammalian cell line of interest with the pcDNA6.2/cGeneBLAzer-GW/D-TOPO® or pcDNA6.2/nGeneBLAzer-GW/D-TOPO® construct using your transfection method of choice.

24 hours after transfection, wash the cells and add fresh growth medium.

48 hours after transfection, split the cells into fresh growth medium such that they are no more than 25% confluent. If the cells are too dense, the antibiotic will not kill the cells. Antibiotics work best on actively dividing cells.

Incubate the cells at 37° C. for 2-3 hours until they have attached to the culture dish.

Remove the growth medium and replace with fresh growth medium containing Blasticidin at the predetermined concentration required for your cell line.

Feed the cells with selective media every 3-4 days until Blasticidin-resistant colonies can be identified.

Pick at least 5 Blasticidin-resistant colonies and expand them to assay for recombinant protein expression.

Detecting Recombinant Fusion Proteins

Introduction

Depending on the kit you are using, you will assay for β-lactamase reporter activity through in vivo or in vitro detection methods. A brief description of each detection method is provided below. For detailed information, refer to the GeneBLAzer Detection Kits manual. If you have generated a pcDNA6.2/nGeneBLAzer-GW/D-TOPO® construct that contains your gene of interest fused to the V5 epitope tag, you may also detect your recombinant fusion protein by Western blot analysis using one of the Anti-V5 Antibodies available from Invitrogen.

In Vitro Detection

Using the GeneBLAzer™ In Vitro Detection Kit allows you to quantitate the amount of intracellular β-lactamase in cells based on the β-lactamase activity in lysates.

To detect β-lactamase activity in mammalian cell lysates, you will use the CCF2-FA substrate. CCF2-FA is the non-esterified, free acid form of CCF2, and is recommended for in vitro use because it is readily soluble in aqueous solution and may be added directly to pre-made cell lysates. Once added to cell lysates, you may quantitate the CCF2-FA fluorescence signal using a fluorescence plate reader or a fluorometer.

To prepare cell lysates from mammalian cells containing the bla(M) reporter gene, you must use a method that will preserve the activity of the β-lactamase enzyme. Refer to the GeneBLAzer™ Detection Kits manual for detailed guidelines and protocols to prepare CCF2-FA solution, prepare cell lysates and samples, and detect CCF2 signal.

In Vivo Detection

Using the GeneBLAzer™ In Vivo Detection Kit allows you to measure β-lactamase reporter activity in live mammalian cells. Once β-lactamase reporter activity has been measured, cells may be cultured further for use in additional assays or other downstream applications.

To detect β-lactamase activity in live mammalian cells, you will use the CCF2-AM substrate. CCF2-AM is the membrane-permeable, esterified form of CCF2, and is recommended for in vivo use because it is non-toxic, lipophilic, and readily enters the cell. Once cells are “loaded” with CCF2-AM, you may quantitate the CCF2 fluorescence signal using a variety of methods.

Refer to the GeneBLAzer Detection Kits manual for detailed guidelines and protocols to prepare CCF2-AM solution, load cells with CCF2-AM substrate, and detect CCF2 signal.

Detecting the V5 Epitope Tag

If you are using pcDNA6.2/nGeneBLAzer-GW/D-TOPO® vector and you have fused your gene of interest to the VS epitope tag, you may detect expression of your recombinant fusion protein using the Anti-V5 Antibody (Catalog no. R960-25), Anti-VS-HRP Antibody (Catalog no. R961-25), or Anti-VS-AP Antibody (Catalog no. R962-25) available from Invitrogen. In addition, the Positope™ Control Protein (Catalog no. R900-50) is available from Invitrogen for use as a positive control for detection of fusion proteins containing a VS epitope. The ready-to-use WesternBreeze® Chromogenic Kits and WesternBreeze® Chemiluminescent Kits are available from Invitrogen to facilitate detection of antibodies by colorimetric or chemiluminescent methods.

Expression of your protein fused to the β-lactamase reporter and/or to the V5 epitope tag will increase the size of your recombinant protein. The table below lists the increase in the molecular weight of your recombinant protein that you should expect from a particular fusion. Note that the expected sizes take into account any additional amino acids between the gene of interest and the fusion peptide.

TABLE 19
Expected Molecular Weight Increases

| | | Expected Size Increase |
|---|---|---|
| Vector | Fusion | (kDa) |
| pcDNA6.2/cGeneBLAzer- | β-lactamase | 31 kDa |
| GW/D-TOPO ® | (C-terminal) | |
| pcDNA6.2/nGeneBLAzer- | β-lactamase | 31 kDa |
| GW/D-TOPO ® | (N-terminal) | |
| | V5 | 3.5 kDa |
| | (C-terminal) | |

Assay for β-Galactosidase

If you use pcDNA m6.2/cGeneBLAzer m-GW/lacZ or pcDNA 6.2/nGeneBLAzer™-GW/lacZ) as a positive control vector, you may assay for β-galactosidase expression by Western blot analysis or activity assay (Miller, J. H. (1972). Experiments in Molecular Genetics (Cold Spring Harbor, N.Y.: Cold Spring Harbor Laboratory). Invitrogen offers β-Gal Antiserum, the β-Gal Assay Kit, and the β-Gal Staining Kit for fast and easy detection of β-galactosidase expression.

Creating an Entry Clone

Introduction

Once you have TOPO® Cloned your gene of interest into a GeneBLAzer™ Directional TOPO® vector, you may perform a BP recombination reaction between your expression construct and a Gateway® donor vector to generate an entry clone. Once you generate an entry clone, your gene of interest may then be easily shuttled into a large selection of destination vectors using the LR recombination reaction. To ensure that you obtain the best possible results, we recommend that you read this section and the next section entitled Performing the BP Recombination Reaction before beginning.

Recombining the Expression Clone with a Donor Vector

Before performing the BP recombination reaction, consider the following points:

The bla(M) reporter gene will not be recombined into the entry clone. If you are using pcDNA6.2/nGeneBLAzer-GW/D-TOPO®, the V5 epitope tag will also not be recombined into the entry clone. If you wish to fuse your gene of interest to any N-terminal or C-terminal peptides, the peptides will need to be provided by the destination vector in the LR recombination reaction.

If you cloned the gene of interest to be in frame with an N-terminal or C-terminal peptide in one of the GeneBLAzer™ Directional TOPO® vectors, the gene will remain in frame with any N-terminal or C-terminal tags provided by the destination vector following the LR recombination reaction.

Depending on the design of your forward and reverse primers, your gene in the entry clone may not contain an ATG initiation codon within the context of a Kozak consensus sequence or a stop codon. If either of these are required, they will need to be provided by the destination vector in the LR recombination reaction.

Experimental Outline

To generate an entry clone, you will:

Perform a BP recombination reaction between your pcDNA6.2/GeneBLAzer-GW/D-TOPO® expression clone and an attP-containing donor vector (see below)

Transform the reaction mixture into a suitable E. coli host

Select for entry clones

Gateway® Donor Vectors

Invitrogen offers a variety of Gateway® donor vectors to help you generate an entry clone containing your gene of interest.

For optimal efficiency, perform the BP recombination reaction using:

Linear pcDNA6.2/GeneBLAzer-GW/D-TOPO® expression clone (see below for guidelines to linearize expression clones)

Supercoiled attP-containing donor vector

Note: Supercoiled or relaxed attB expression clones may be used, but will react less efficiently than linear attB expression clones.

Linearizing Expression Clones

We recommend that you linearize your pcDNA6.2/GeneBLAzer-GW/D-TOPO®expression clone using a suitable restriction enzyme (see the guidelines below).

Linearize 1 to 2 μg of the expression clone with a unique restriction enzyme that does not digest within the gene of interest and is located outside the attB region. Ethanol precipitate the DNA after digestion by adding 0.1 volume of 3 M sodium acetate followed by 2.5 volumes of 100% ethanol.

Pellet the DNA by centrifugation. Wash the pellet twice with 70% ethanol.

Dissolve the DNA in TE Buffer, pH 8.0 to a final concentration of 50-150 ng/μl.

Performing the BP Recombination Reaction

Introduction

General guidelines and instructions are provided in this section to perform a BP recombination reaction using your pcDNA6.2/GeneBLAzer-GW/D-TOPO® expression clone and a donor vector, and to transform the reaction mixture into a suitable E. coli host to select for entry clones. We recommend that you include a positive control (see below) in your experiment to help you evaluate your results.

Positive Control

pEXP7-tet is provided with the BP Clonase™ enzyme mix as a positive control for the BP reaction. pEXP7-tet is an approximately 1.4 kb linear fragment and contains attB sites flanking the tetracycline resistance gene and its promoter (Tcr). Using the pEXP7-tet fragment in a BP reaction with a donor vector results in entry clones that express the tetracycline resistance gene. The efficiency of the BP recombination reaction can easily be determined by streaking entry clones onto LB plates containing 20 μg/ml tetracycline.

Determining How Much DNA to Use

For optimal efficiency, we recommend using the following amounts of linearized attB expression clone and donor vector in a 20 μl BP recombination reaction:

An equimolar amount of linearized attB expression clone and the donor vector

100 femtomoles (fmol) each of linearized attB expression clone and donor vector is preferred, but the amount of attB expression clone used may range from 40-100 fmol

Note: 100 fmol of donor vector (pDONR™201, pDONR™221, or pDONR™/Zeo) is approximately 300 ng

For a formula to convert fmol of DNA to nanograms (ng), see below.

Do not use more than 500 ng of donor vector in a 20 μl BP reaction as this will affect the efficiency of the reaction

Do not exceed more than 1 pg of total DNA (donor vector plus attB expression clone) in a 20 μl BP reaction as excess DNA will inhibit the reaction

Converting Femtomoles fmol) to Nanograms (ng)

Use the following formula to convert femtomoles (fmol) of DNA to nanograms (ng) of DNA:

$$ng = ( fmol ) ( N ) ( \frac{660 fg}{fmol} ) ( \frac{1 ng}{{10}^{6} fg} )$$

where N is the size of the DNA in bp.

Example of fmol to ng Conversion

In this example, you need to use 100 fmol of your pcDNA6.2/GeneBLAzer-GW/D-TOPO® expression clone which is 7.5 kb in size in the BP reaction. Calculate the amount of your pcDNA6.2/GeneBLAzer-GW/D-TOPO® expression clone required for the reaction (in ng) by using the equation above:

$$( 100 fmol ) ( 7500 bp ) ( \frac{660 fg}{fmol} ) ( \frac{1 ng}{{10}^{6 } fg} ) = 495 ng of expression clone required$$

Materials Needed

You should have the following materials on hand before beginning:

Linearized pcDNA6.2/GeneBLAzer-GW/D-TOPO® expression clone pDONR™ vector (resuspended to 150 ng/1)

BP Clonase™ enzyme mix

5×BP Clonase™ Reaction Buffer (supplied with the BP Clonase™ enzyme mix) pEXP7-tet positive control, optional (50 ng/μl; supplied with the BP Clonase™ enzyme mix)

TE Buffer, pH 8.0 (10 mm Tris-HCl, pH 8.0; 1 mM EDTA) 2 μg/μl Proteinase K solution (supplied with the BP Clonase™ enzyme mix; thaw and keep on ice until use)

Appropriate competent E. coli host and growth media for expression

S.O.C. medium

LB agar plates containing the appropriate antibiotic to select for entry clones

Setting Up the BP Recombination Reaction

Add the following components to 1.5 ml microcentrifuge tubes at room temperature and mix.

Note: To include a negative control, set up a second sample reaction and substitute TE Buffer, pH 8.0 for the BP Clonase™ enzyme mix (see Step 4).

TABLE 20
BP Recombination Reaction

| Components | Sample | | Positive Control |
|---|---|---|---|
| pcDNA6.2/GeneBLAzer-GW/D-TOPO ® | 1-10 | μl | — |
| expression clone (40-100 fmol) | | | |
| pDONR ™ vector (150 ng/μl) | 2 | μl | 2 μl |
| pEXP7-tet positive control (50 ng/μl) | — | | 2 μl |
| 5× BP Clonase ™ Reaction Buffer | 4 | μl | 4 μl |
| TE Buffer, pH 8.0 | to 16 | μl | 8 μl |

Remove the BP Clonase™ enzyme mix from −80° C. and thaw on ice (˜2 minutes).

Vortex the BP Clonase™ enzyme mix briefly twice (2 seconds each time).

To each sample above, add 4 μl of BP Clonase™ enzyme mix. Mix well by vortexing briefly twice (2 seconds each time).

Reminder: Return BP Clonase™ enzyme mix to −80° C. immediately after use.

Incubate reactions at 25° C. for 1 hour.

Note: For most applications, a 1 hour incubation will yield a sufficient number of entry clones. Depending on your needs, the length of the recombination reaction can be extended up to 18 hours. An overnight incubation typically yields 5-10 times more colonies than a 1 hour incubation.

Add 2 μl of the Proteinase K solution to each reaction. Incubate for 10 minutes at 37° C.

Transform 1 μl of the BP recombination reaction into a suitable E. coli host (follow the manufacturer's instructions) and select for entry clones.

Note: You may store the BP reaction at −20° C. for up to 1 week before

transformation,

if desired.

What You Should See

If you use E. coli cells with a transformation efficiency of 1×10⁸ cfu/μg, the BP recombination reaction should give >1500 colonies if the entire BP reaction is transformed and plated.

Verifying pEXP7-tet Entry Clones

If you included the pEXP7-tet control in your experiments, you may access the efficiency of the BP reaction by streaking entry clones onto LB plates containing 20 μg/ml tetracycline. True entry clones should be tetracycline-resistant.

Troubleshooting

TOPO® Cloning Reaction and Transformation

The table below lists some potential problems and possible solutions that may help you troubleshoot the TOPO® Cloning and transformation reactions. To help evaluate your results, we recommend that you perform the control reactions in parallel with your samples.

TABLE 21
Potential Problems, Reasons and Solutions

| Problem | Reason | Solution |
|---|---|---|
| Few or no | Suboptimal ratio of PCR | Use a 0.5:1 to 2:1 molar ratio |
| colonies | product:TOPO ® vector | of PCR product:TOPO ® |
| obtained from | used in the TOPO ® | vector. |
| sample reactio | Cloning reaction | |
| and then | Too much PCR product | Dilute the PCR product. |
| transformation | used in the TOPO ® | Use a 0.5:1 to 2:1 molar ratio |
| control gave | Cloning reaction | of PCR product:TOPO ® |
| | | vector. |
| colonies | PCR product too dilute | Concentrate the PCR product. |
| | | Use a 0.5:1 to 2:1 molar ratio |
| | | of PCR product:TOPO ® |
| | | vector. |
| | PCR primers contain 5′ | Do not add 5′ phosphates to |
| | phosphates | your PCR primers. |
| | Incorrect PCR primer | Make sure that the forward |
| | design | PCR primer contains the |
| | | sequence CACC at the 5′ end. |
| | | Make sure that the reverse |
| | | PCR primer does not contain |
| | | the sequence CACC at the 5′ |
| | | end. |
| | Used Taq polymerase or | Use a proofreading |
| | a Taq/proofreading | polymerase for PCR. |
| | polymerase mixture | |
| | for PCR | |
| | Long PCR product | Increase the incubation time |
| | | of the TOPO ® reaction from |
| | | 5 minutes to 30 minutes. |
| | | Gel-purify the PCR product |
| | | to remove primer-dimers and |
| | | other artifacts. |
| | PCR reaction contains | Optimize your PCR using the |
| | artifacts (i.e. does not | proofreading polymerase of |
| | run as a single, discrete | choice. |
| | band on an agarose gel) | Gel-purify your PCR product |
| | | to remove primer-dimers and |
| | | smaller PCR products. |
| | Cloning large pool of | Increase the incubation time |
| | PCR products or a | of the TOPO ® reaction from |
| | toxic gene | 5 minutes to 30 minutes. |
| | | Use a 0.5:1 to 2:1 molar ratio |
| | | of PCR product:TOPO ® |
| | | vector. |

TABLE 22
Potential Problems, Reasons and Solutions

| Problem | Reason | Solution |
|---|---|---|
| Large number of | PCR reaction contains artifacts | Optimize your PCR using the |
| incorrect inserts | (i.e. does not run as a single, | proofreading polymerase of |
| cloned | discrete band on an agarose gel) | choice. |
| | | Gel-purify your PCR product |
| | | to remove primer-dimers and |
| | | smaller PCR products. |
| | Incorrect PCR primer design | Make sure that the forward |
| | | PCR primer contains the |
| | | sequence CACC at the 5′ end. |
| | | Make sure that the reverse |
| | | PCR primer does not contain |
| | | the sequence CACC at the 5′ end. |
| Few or no | One Shot ® competent E. coli | Store One Shot ® competent |
| colonies obtained | stored incorrectly | E. coli at −80° C. |
| from sample | | If you are using another E. coli |
| reaction and the | | strain, follow the |
| transformation | | manufacturer's instructions. |
| control gave no | One Shot ® transformation | Follow the One Shot ® |
| colonies | protocol not followed correctly | transformation protocol. |
| | Insufficient amount of E. coli | Increase the amount of E. coli |
| | plated | plated. |
| | Selective plates not prewarmed | Warm selective plates at 37° C. |
| | before spreading | for 30 minutes prior to |
| | | spreading. |
| | Transformants plated on | Use the appropriate antibiotic |
| | selective plates containing the | for selection. |
| | wrong antibiotic | |
| No visible | Not using ampicillin selection | Colonies will appear 8 hours |
| colonies 8 hours | | after plating with ampicillin |
| after plating | | selection. If you are using |
| transformed | | Blasticidin selection, incubate |
| Mach1 ™-T1^{R} | | plates overnight at 37° C. |
| cells | Selective plates not prewarmed | Warm selective plates at 37° C. |
| | before spreading | for 30 minutes prior to spreading. |

Performing the Control Reactions

Introduction

We recommend performing the following control TOPO® Cloning reactions the first time you TOPO® Clone to help you evaluate your results. Performing the control reactions involves producing a control PCR product using the reagents included in the kit and using this product directly in a TOPO® Cloning reaction.

### Before Starting

For each transformation, prepare two LB plates containing 50-100 μg/ml ampicillin.

### Producing the Control PCR Product

Use your thermostable, proofreading polymerase and the appropriate buffer to amplify the control PCR product. Follow the manufacturer's recommendations for the polymerase you are using.

1. To produce the 750 bp control PCR product, set up the following 50 μl PCR:
Control DNA Template (100 ng) 1 μl
10×PCR Buffer (appropriate for enzyme) 5 μl
dNTP Mix 0.5 PI
Control PCR Primers (0.1 μg/μl each) 1 μl
Sterile Water 41.5 μl
Thermostable polymerase (1-2.5 units/μl) 1 μl
Total Volume 50 μl

2. Overlay with 70 μl (1 drop) of mineral oil, if required.

3. Amplify using the following cycling parameters:

TABLE 23
Cycling Parameters

| Step | Time | Temperature | Cycles |
|---|---|---|---|
| Initial Denaturation | 2 minutes | 94° C. | 1X |
| Denaturation | 1 minute | 94° C. | 25X |
| Annealing | 1 minute | 55° C. | |
| Extension | 1 minute | 72° C. | |
| Final Extension | 7 minutes | 72° C. | 1X |

Remove 10 μl from the reaction and analyze by agarose gel electrophoresis. A discrete 750 bp band should be visible.

Estimate the concentration of the PCR product, and adjust as necessary such that the amount of PCR produce used in the control TOPO® Cloning reaction results in an optimal molar ratio of PCR product:TOPO® vector (i.e. 0.5:1 to 2:1). Proceed to Control TOPO® Cloning Reactions.

Performing the Control Reactions, continued

Control TOPO® Cloning Reactions

Using the control PCR product produced and a pcDNA6.2/GeneBLAzer-GW/D-TOPO® vector, set up two 6 μl TOPO® Cloning reactions as described below. If you plan to transform electrocompetent E. coli, use Dilute Salt Solution in place of the Salt Solution.

Set up control TOPO® Cloning reactions:

TABLE 24
TOPO Cloning Reactions

| | “Vector | “Vector + PCR |
|---|---|---|
| Reagent | Only” | Insert” |
| Sterile Water | 4 μl | 3 μl |
| Salt Solution | 1 μl | 1 μl |
| Control PCR Product | — | 1 μl |
| pcDNA6.2/GeneBLAzer- | 1 μl | 1 μl |
| GW/D-TOPO ® | | |
| Final volume | 6 μl | 6 μl |

Incubate at room temperature for 5 minutes and place on ice.

Transform 2 μl of each reaction into separate vials of One Shot® Mach1™-T1® cells using the protocol.

Spread 50-200 μl of each transformation mix onto LB plates containing 50-100 μg/ml ampicillin. Be sure to plate two different volumes to ensure that at least one plate has well-spaced colonies.

Incubate overnight at 37° C.

Transformation Control

pUC19 plasmid is included to check the transformation efficiency of the One Shot® Mach™-T1® competent cells. Transform one vial of One Shot® Mach1™-T1® cells with 10 pg of pUC19 using the protocol. Plate 10 μl of the transformation mixture plus 20 μl of S.O.C. medium on LB plates containing 100 μg/ml ampicillin. Transformation efficiency should be ˜1×10⁹ cfu/μg DNA.

Analysis of Results

Hundreds of colonies from the vector+PCR insert reaction should be produced. To analyze the transformations, isolate plasmid DNA and digest with the appropriate restriction enzyme as listed below. Refer to the table below for expected digestion patterns.

TABLE 25
Expected Digestion Paterns

| | Restriction | |
|---|---|---|
| Vector | Enzyme | Expected Digestion Patterns (bp) |
| pcDNA6.2/cGeneBLAzer- | Ava I | Correct orientation: 4032, 2617 |
| GW/D-TOPO ® | | Reverse orientation: 4603, 2046 |
| | | Empty vector: 5900 |
| pcDNA6.2/nGeneBLAzer- | Ava I | Correct orientation: 4829, 1865 |
| GW/D-TOPO ® | | Reverse orientation: 5400, 1294 |
| | | Empty vector: 5945 |

Greater than 90% of the colonies should contain the 750 bp insert in the correct orientation. Relatively few colonies should be produced in the vector-only reaction.

Gel Purifying PCR Products

Introduction

Smearing, multiple banding, primer-dimer artifacts, or large PCR products (>3 kb) may necessitate gel purification. If you wish to purify your PCR product, be extremely careful to remove all sources of nuclease contamination. There are many protocols to isolate DNA fragments or remove oligonucleotides. Refer to Current Protocols in Molecular Biology, Unit 2.6 (Ausubel, F. M., Brent, R., Kingston, R. E., Moore, D. D., Seidman, J. G., Smith, J. A., and Struhl, K. (1994). Current Protocols in Molecular Biology (New York: Greene Publishing Associates and Wiley-Interscience) for the most common protocols. Three simple protocols are provided below. The cloning efficiency may decrease with purification of the PCR product (e.g. PCR product too dilute). You may wish to optimize your PCR to produce a single band (see Producing Blunt-End PCR Products).

Using the S.N.A.P.™ Gel Purification Kit

The S.N.A.P.™ Gel Purification Kit available from Invitrogen (Catalog no. K1999-25) allows you to rapidly purify PCR products from regular agarose gels.

1. Electrophorese amplification reaction on a 1 to 5% regular TAE agarose gel.

Note: Do not use TBE to prepare agarose gels. Borate interferes with the sodium iodide step, below.

2. Cut out the gel slice containing the PCR product and melt it at 65° C. in 2 volumes of the 6 M sodium iodide solution.

3. Add 1.5 volumes Binding Buffer.

4. Load solution (no more than 1 ml at a time) from Step 3 onto a S.N.A.P.™ column. Centrifuge 1 minute at 3000×g in a microcentrifuge and discard the supernatant.

5. If you have solution remaining from Step 3, repeat Step 4.

6. Add 900111 of the Final Wash Buffer.

7. Centrifuge 1 minute at full speed in a microcentrifuge and discard the flow-through.

8. Repeat Step 7.

9. Elute the purified PCR product in 40 μl of TE or sterile water. Use 4 μl for the TOPO® Cloning reaction and proceed.

Quick S.N.A.P.™ Method

An even easier method is to simply cut out the gel slice containing your PCR product, place it on top of the S.N.A.P. column bed, and centrifuge at full speed for 10 seconds. Use 1-2 μl of the flow-through in the TOPO® Cloning reaction. Be sure to make the gel slice as small as possible for best results.

Gel Purifying PCR Products, continued

Low-Melt Agarose Method

If you prefer to use low-melt agarose, use the procedure below. Note that gel purification will result in a dilution of your PCR product and a potential loss of cloning efficiency.

1. Electrophorese as much as possible of your PCR reaction on a low-melt agarose gel (0.8 to 1.2%) in TAE buffer.

2. Visualize the band of interest and excise the band.

3. Place the gel slice in a microcentrifuge tube and incubate the tube at 65° C. until the gel slice melts.

4. Place the tube at 37° C. to keep the agarose melted.

5. Add 4 μl of the melted agarose containing your PCR product to the TOPO® Cloning reaction as described.

6. Incubate the TOPOI Cloning reaction at 37° C. for 5 to 10 minutes. This is to keep the agarose melted.

7. Transform 2 to 4 μl directly into One Shote Mach1™-T1® cells.

The cloning efficiency may decrease with purification of the PCR product. You may wish to optimize your PCR to produce a single band.

Recipes

LB (Luria-Bertani) Medium and Plates

1.0% Tryptone

0.5% Yeast Extract

1.0% NaCl

pH 7.0

For 1 liter, dissolve 10 g tryptone, 5 g yeast extract, and 10 g NaCl in 950 ml deionized water.

Adjust the pH of the solution to 7.0 with NaOH and bring the volume up to 1 liter.

Autoclave on liquid cycle for 20 minutes at 15 psi. Allow solution to cool to 55° C. and add antibiotic (50-100 μg/ml ampicillin) if needed.

Store at room temperature or at +4° C.

LB Agar Plates

Prepare LB medium as above, but add 15 g/L agar before autoclaving.

Autoclave on liquid cycle for 20 minutes at 15 psi.

After autoclaving, cool to 55° C., add antibiotic (50-100 μg/ml of ampicillin), and pour into 10 cm plates.

Let harden, then invert and store at +4° C.

Low Salt LB Medium with Blasticidin

### Low Salt LB Medium:

10 g Tryptone

5 g NaCl

5 g Yeast Extract

Combine the dry reagents above and add deionized, distilled water to 950 ml. Adjust pH to 7.0 with 1 N NaOH. Bring the volume up to 1 liter. For plates, add 15 g/L agar before autoclaving.

Autoclave on liquid cycle at 15 psi and 121° C. for 20 minutes.

Allow the medium to cool to at least 55° C. before adding the Blasticidin to 100 μg/ml final concentration.

Store plates at +4° C. in the dark. Plates containing Blasticidin are stable for up to 2 weeks.

Blasticidin

Blasticidin S HCl is a nucleoside antibiotic isolated from Streptomyces griseochromogenes which inhibits protein synthesis in both prokaryotic and eukaryotic cells (Takeuchi, S., Hirayama, K., Ueda, K., Sakai, H., and Yonehara, H. (1958). Blasticidin S, A New Antibiotic. The Journal of Antibiotics, Series A 11, 1-5; Yamaguchi, H., Yamamoto, C., and Tanaka, N. (1965). Inhibition of Protein Synthesis by Blasticidin S. I. Studies with Cell-free Systems from Bacterial and Mammalian Cells. J. Biochem (Tokyo) 57, 667-677). Resistance is conferred by expression of either one of two Blasticidin S deaminase genes: bsdfrom Aspergillus terreus (Kimura, M., Takatsuki, A., and Yamaguchi, I. (1994). Blasticidin S Deaminase Gene from Aspergillus terreus (BSD): A New Drug Resistance Gene for Transfection of Mammalian Cells. Biochim. Biophys. ACTA 1219, 653-659) or bsr from Bacillus cereus (Izumi, M., Miyazawa, H., Kamakura, T., Yamaguchi, I., Endo, T., and Hanaoka, F. (1991). Blasticidin S-Resistance Gene (bsr): A Novel Selectable Marker for Mammalian Cells. Exp. Cell Res. 197, 229-233). These deaminases convert Blasticidin S to a non-toxic deaminohydroxy derivative (Izumi, M., Miyazawa, H., Kamakura, T., Yamaguchi, I., Endo, T., and Hanaoka, F. (1991). Blasticidin S-Resistance Gene (bsr): A Novel Selectable Marker for Mammalian Cells. Exp. Cell Res. 197, 229-233).

Molecular Weight, Formula, and Structure

The formula for Blasticidin S is C₁₇H₂₆N₈O₅—HCl, and the molecular weight is 458.9. The diagram below shows the structure of Blasticidin.

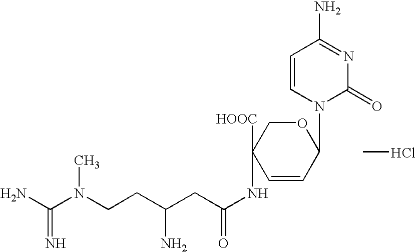

Handling Blasticidin

Always wear gloves, mask, goggles, and protective clothing (e.g. a laboratory coat) when handling Blasticidin. Weigh out Blasticidin and prepare solutions in a hood.

### Preparing and Storing Stock Solutions

Blasticidin may be obtained separately from Invitrogen (Catalog no. R210-01) in 50 mg aliquots. Blasticidin is soluble in water. Sterile water is generally used to prepare stock solutions of 5 to 10 mg/ml.
Dissolve Blasticidin in sterile water and filter-sterilize the solution.
Aliquot in small volumes suitable for one time use (see next to last point below) and freeze at −20° C. for long-term storage or store at +4° C. for short-term storage.
Aqueous stock solutions are stable for 1-2 weeks at +4° C. and 6-8 weeks at −20° C.
pH of the aqueous solution should be 7.0 to prevent inactivation of Blasticidin.
Do not subject stock solutions to freeze/thaw cycles (do not store in a frost-free freezer).
Upon thawing, use what you need and store the thawed stock solution at +4° C. for up to 2 weeks.
Medium containing Blasticidin may be stored at +4° C. for up to 2 weeks.
   Features of pcDNA6.2/cGeneBLAzer-GW/D-TOPO®

pcDNA6.2/cGeneBLAzer-GW/D-TOPO® (5900) contains the following elements. All features have been functionally tested.

TABLE 26
Features of pcDNA6.2/cGeneBLAzer-GW/D-TOPO ®

| Feature | Benefit |
|---|---|
| Human cytomegalovirus | Allows efficient, high-level expression of your |
| (CMV) immediate-early | recombinant protein (Andersson, S., Davis, D. L., |
| promoter/enhancer | Dahlback, H., Jornvall, H., and Russell, D. W. |
| | (1989). Cloning, Structure, and Expression of the |
| | Mitochondrial Cytochrome P-450 Sterol 26- |
| | Hydroxylase, a Bile Acid Biosynthetic Enzyme. J. |
| | Biol. Chem. 264, 8222-8229; Boshart, M., Weber, F., |
| | Jahn, G., Dorsch-Hasler, K., Fleckenstein, B., |
| | and Schaffner, W. (1985). A Very Strong Enhancer |
| | is Located Upstream of an Immediate Early Gene |
| | of Human Cytomegalovirus. Cell 41, 521-530; |
| | Nelson, J. A., Reynolds-Kohler, C., and Smith, B. A. |
| | (1987). Negative and Positive Regulation by a |
| | Short Segment in the 5′-Flanking Region of the |
| | Human Cytomegalovirus Major Immediate-Early |
| | Gene. Molec. Cell. Biol. 7, 4125-4129) |
| T7 promoter/priming site | Allows in vitro transcription in the sense |
| | orientation and sequencing through the insert |
| attB1 and attB2 sites | Allows recombinational cloning of the gene of |
| | interest to generate an entry clone |
| TOPO ® Cloning site | Allows directional cloning of your PCR product in |
| (directional) | frame with the C-terminal β-lactamase reporter gene |
| β-lactamase bla(M) | Allows fusion of the β-lactamase reporter to the C- |
| reporter gene | terminus of your protein for use as a reporter of |
| | gene expression (Zlokarnik, G., Negulescu, P. A., |
| | Knapp, T. E., Mere, L., Burres, N., Feng, L., |
| | Whitney, M., Roemer, K., and Tsien, R. Y. (1998). |
| | Quantitation of Transcription and Clonal Selection |
| | of Single Living Cells with b-Lactamase as |
| | Reporter. Science 279, 84-888) |
| Herpes Simplex Virus | Allows efficient transcription termination and |
| Thymidine Kinase (TK) | polyadenylation of mRNA (Cole, C. N., and Stacy, T. P. |
| polyadenylation signal | (1985). Identification of Sequences in the |
| | Herpes Simplex Virus Thymidine Kinase Gene |
| | Required for Efficient Processing and |
| | Polyadenylation. Mol. Cell. Biol. 5, 2104-2113) |
| f1 origin | Allows rescue of single-stranded DNA |
| SV40 early promoter and origin | Allows efficient, high-level expression of the |
| | Blasticidin resistance gene and episomal |
| | replication in cells expressing the SV40 large T antigen |
| EM7 promoter | Allows expression of the Blasticidin resistance |
| | gene in E. coli |
| Blasticidin (bsd) | Allows selection of stable transfectants in |
| resistance gene | mammalian cells (Kimura, M., Takatsuki, A., and |
| | Yamaguchi, I. (1994). Blasticidin S Deaminase |
| | Gene from Aspergillus terreus (BSD): A New Drug |
| | Resistance Gene for Transfection of Mammalian |
| | Cells. Biochim. Biophys. ACTA 1219, 653-659) |
| SV40 early | Allows efficient transcription termination and |
| polyadenylation signal | polyadenylation of mRNA |
| pUC origin | Allows high-copy number replication and growth |
| | in E. coli |
| Ampicillin resistance gene | Allows selection of transformants in E. coli |

Features of pcDNA6.2/nGeneBLAzer-GW/D-TOPO®

pcDNA6.2/nGeneBLAzer-GW/D-TOPO® (5945) contains the following elements. All features have been functionally tested.

TABLE 27
Features of pcDNA6.2/nGeneBLAzer-GW/D-TOPO ®

| Feature | Benefit |
|---|---|
| Human cytomegalovirus | Allows efficient, high-level expression of your |
| (CMV) immediate-early | recombinant protein (Andersson, S., Davis, D. L., |
| promoter/enhancer | Dahlback, H., Jornvall, H., and Russell, D. W. |
| | (1989). Cloning, Structure, and Expression of the |
| | Mitochondrial Cytochrome P-450 Sterol 26- |
| | Hydroxylase, a Bile Acid Biosynthetic Enzyme. J. |
| | Biol. Chem. 264, 8222-8229; Boshart, M., Weber, F., |
| | Jahn, G., Dorsch-Hasler, K., Fleckenstein, B., |
| | and Schaffner, W. (1985). A Very Strong Enhancer |
| | is Located Upstream of an Immediate Early Gene |
| | of Human Cytomegalovirus. Cell 41, 521-530; |
| | Nelson, J. A., Reynolds-Kohler, C., and Smith, B. A. |
| | (1987). Negative and Positive Regulation by a |
| | Short Segment in the 5′-Flanking Region of the |
| | Human Cytomegalovirus Major Immediate-Early |
| | Gene. Molec. Cell. Biol. 7, 4125-4129) |
| T7 promoter | Allows in vitro transcription in the sense |
| | orientation |
| β-lactamase bla(M) | Allows fusion of the β-lactamase reporter to the N- |
| reporter gene | terminus of your protein for use as a reporter of |
| | gene expression (Zlokarnik, G., Negulescu, P. A., |
| | Knapp, T. E., Mere, L., Burres, N., Feng, L., |
| | Whitney, M., Roemer, K., and Tsien, R. Y. (1998). |
| | Quantitation of Transcription and Clonal Selection |
| | of Single Living Cells with b-Lactamase as |
| | Reporter. Science 279, 84-888) |
| attB1 and attB2 sites | Allows recombinational cloning of the gene of |
| | interest to generate an entry clone |
| TOPO ® Cloning site | Allows directional cloning of your PCR product in |
| (directional) | frame with the N-terminal β-lactamase reporter |
| | gene |
| V5 epitope | Allows detection of the recombinant fusion protein |
| | by the Anti-V5 antibodies (Southern, J. A., Young, D. F., |
| | Heaney, F., Baumgartner, W., and Randall, R. E. |
| | (1991). Identification of an Epitope on the P |
| | and V Proteins of Simian Virus 5 That |
| | Distinguishes Between Two Isolates with Different |
| | Biological Characteristics. J. Gen. Virol. 72, 1551-1557). |
| Herpes Simplex Virus | Allows efficient transcription termination and |
| Thymidine Kinase (TK) | polyadenylation of mRNA (Cole, C. N., and Stacy, T. P. |
| polyadenylation signal | (1985). Identification of Sequences in the |
| | Herpes Simplex Virus Thymidine Kinase Gene |
| | Required for Efficient Processing and |
| | Polyadenylation. Mol. Cell. Biol. 5, 2104-2113) |
| TK polyA reverse | Allow sequencing through the insert |
| priming site | |
| f1 origin | Allows rescue of single-stranded DNA |
| SV40 early promoter and | Allows efficient, high-level expression of the |
| origin | Blasticidin resistance gene and episomal |
| | replication in cells expressing the SV40 large T |
| | antigen |
| EM7 promoter | Allows expression of the Blasticidin resistance |
| | gene in E. coli |
| Blasticidin (bsd) | Allows selection of stable transfectants in |
| resistance gene | mammalian cells (Kimura, M., Takatsuki, A., and |
| | Yamaguchi, I. (1994). Blasticidin S Deaminase |
| | Gene from Aspergillus terreus (BSD): A New Drug |
| | Resistance Gene for Transfection of Mammalian |
| | Cells. Biochim. Biophys. ACTA 1219, 653-659) |
| SV40 early | Allows efficient transcription termination and |
| polyadenylation signal | polyadenylation of mRNA |
| pUC origin | Allows high-copy number replication and growth |
| | in E. coli |
| Ampicillin resistance gene | Allows selection of transformants in E. coli |

### Example 11

### Exemplary Product Instructions

The following example is intended to illustrate exemplary methods for carrying out the present invention. Variations on the methods set forth herein will be readliy appreciated by those skilled in the art. The information set forth in this or any other example should not be construed as limiting the scope of the invention described herein. All catalog numbers mentioned in this example refer to specific products and reagents avaliable from Invitrogen Corporation, Carlsbad, Calif., 92008. The exemplary methods described in this example can be carried out using the products and reagents designated by the catalog numbers, or with equivalent products and reagents available from other sources.

Accessory Products

Additional Products Additional products that may be used with pENTR/GeneBLAzer™ are available from Invitrogen.

TABLE 28
Additional Products

| | | Catalog |
|---|---|---|
| Item | Quantity | no. |
| GeneBLAzer ™ In Vitro | 100 μg | 12578- |
| Detection Kit | | 126 |
| GeneBLAzer ™ In Vivo | 50 μg | 12578- |
| Detection Kit | | 134 |
| Gateway ® LR Clonase ™ | 20 reactions | 11791- |
| Enzyme Mix | | 019 |
| | 100 | 11791- |
| | reactions | 043 |
| One Shot ® TOP10 Chemically | 10 reactions | C4040-10 |
| Competent Cells | 20 reactions | C4040-03 |
| One Shot ® TOP10 | 10 reactions | C4040-50 |
| Electrocompetent Cells | 20 reactions | C4040-52 |
| Kanamycin Sulfate | 1 g | 11815- |
| | | 016 |

Gateway® Destination Vectors

A large selection of Gateway® destination vectors is available from Invitrogen to facilitate expression of your gene of interest in virtually any protein expression system.

Overview

Introduction

pENTR/GeneBLAzer™ is a Gateway® entry clone containing the β-lactamase gene. Following recombination with a mammalian Gateway® destination vector to generate an expression control, β lactamase activity can be detected in vivo or in vitro using GeneBLAzer™ Technology. Detection of β lactamase activity allows you to optimize transfection and expression studies, normalize for experimental variability, and provides a highly sensitive and accurate method to quantitate gene expression in mammalian cells.

Features of pENTR/GeneBLAzer™

pENTR/GeneBLAzer™ contains the following elements:

rrnB transcription termination sequences to prevent basal expression of the β-lactamase gene in E. coli

attL1 and attL2 sites for site-specific recombination of the entry clone with a Gateway® destination vector

Kozak consensus sequence for efficient translation initiation in eukaryotic systems

β-lactamase bla(M) gene for in vivo or in vitro fluorescence detection

Kanamycin resistance gene for selection in E. coli

pUC origin for high-copy replication and maintenance of the plasmid in E. coli

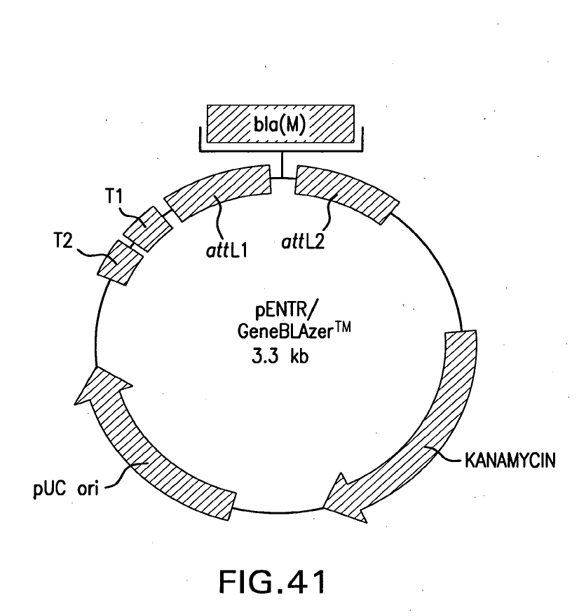

For a map of pENTR/GeneBLAzer™, refer to FIG. 41.

The Gateway® Technology

Gateway® is a universal cloning technology that takes advantage of the site-specific recombination properties of bacteriophage lambda (Landy, 1989) to provide a rapid and highly efficient way to move your gene of interest into multiple vector systems. To express the bla(M) gene in mammalian cells using Gateway® Technology, simply:

Generate an expression clone by performing an LR recombination reaction between pENTR/GeneBLAzer™ and a mammalian Gateway® destination vector of choice.

Transfect your expression clone into the cell line of choice and assay for transient expression of the bla(M) gene. Generate a stable cell line, if desired.

Advantages of pENTR/GeneBLAzer™

Using pENTR/GeneBLAzer™ and the GeneBLAzer™ Technology as a control for gene expression in mammalian cells provides the following advantages:

β-lactamase activity is detectable in living mammalian cells using fluorescence microscopy.

Provides a ratiometric readout to minimize differences due to variability in cell number, substrate concentration, fluorescence intensity, and emission sensitivity.

Compatible with a wide variety of in vivo and in vitro applications including microplate-based transcriptional assays and flow cytometry.

Using a non-toxic substrate allows continued cell culturing after quantitative analysis.

The GeneBLAzer™ Technology

Components of the GeneBLAzer™ System

The GeneBLAzer™ System facilitates fluorescence detection of β-lactamase activity in mammalian cells, and consists of two major components:

The β-lactamase gene, bla(M), a truncated form of the E. coli bla gene.

A fluorescence resonance energy transfer (FRET)-enabled substrate, CCF2 to facilitate fluorescence detection of β lactamase activity. In the absence or presence of β lactamase activity, cells loaded with the CCF2 substrate fluoresce green or blue, respectively. Comparing the ratio of blue to green fluorescence in a population of live cells or in a cell extract of your sample to a negative control provides a means to quantitate gene expression.

β-Lactamase (bla) Gene

β-lactamase is the product encoded by the ampicillin (bla) resistance gene and is the bacterial enzyme that hydrolyzes penicillins and cephalosporins. The bla gene is present in many cloning vectors and allows ampicillin selection in E. coli. β lactamase enzyme activity is not found in mammalian cells.

bLa(M) Gene

The GeneBLAzer™ Technology uses a modified bla gene for

expression in mammalian cells. This bla gene is derived from the E. coli TEM-1 gene present in many cloning vectors (Zlokarnik, G., Negulescu, P. A., Knapp, T. E., Mere, L., Burres, N., Feng, L., Whitney, M., Roemer, K., and Tsien, R. Y. (1998). Quantitation of Transcription and Clonal Selection of Single Living Cells with b-Lactamase as Reporter. Science 279, 84-88), and has been modified in the following ways:

72 nucleotides encoding the first 24 amino acids of β lactamase were deleted from the N-terminal region of the gene. These 24 amino acids comprise the bacterial periplasmic signal sequence, and deleting this region allows cytoplasmic expression of β-lactamase in mammalian cells.

The amino acid at position 24 was mutated from His to Asp to create an optimal Kozak sequence for optimal translation initiation.

This modified gene is named bla(M).

Note: The TEM-1 gene also contains 2 mutations (at nucleotide positions 452 and 753) that distinguish it from the bla gene in pBR322 (Sutcliffe, J. G. (1978). Nucleotide Sequence of the Ampicillin Resistance Gene of Escherichia coli Plasmid pBR322. Proc. Nat. Acad. Sci. USA 75, 3737-3741).

Methods

Creating an Expression Clone

Introduction

You will need to perform an LR recombination reaction to transfer the β-lactamase gene to your Gateway® destination vector of choice. To ensure that you obtain the best possible results, we recommend that you read this section and the next section entitled Performing the LR Recombination Reaction before beginning.

Resuspending pENTR/GeneBLAzer™

pENTR/GeneBLAzer™ is supplied as 10 μg of plasmid, lyophilized in TE, pH 8.0. To use, resuspend the vector in 100 μl of sterile water to a final concentration of 100 ng/μl.

### Tag-On-Demand™ System

The bla(M) gene in pENTR/GeneBLAzer™ contains a TAG (amber stop) codon, making it compatible with the Tag-On-Demand™ System which allows expression of both native and C-terminally-tagged recombinant protein from the same expression construct.

The System is based on stop suppression technology originally developed by RajBhandary and colleagues (Capone, J. P., Sharp, P. A., and RajBhandary, U. L. (1985). Amber, Ochre and Opal Suppressor tRNA Genes Derived from a Human Serine tRNA Gene. EMBO J. 4, 213-221) and consists of a recombinant adenovirus expressing a tRNAser suppressor. Following an LR recombination reaction with pENTR/GeneBLAzer™ and a destination vector containing a C-terminal tag, the bla(M) gene in the resulting expression clone will retain the TAG stop codon and will be fused in frame to the C-terminal tag. When the expression clone is transfected into mammalian cells and the tRNAser suppressor supernatant is present, the stop codon will be translated as serine, allowing translation to continue and resulting in production of C terminally-tagged P lactamase protein.

Recombination Region

Note the following features:

The bla(M) gene contains a Kozak consensus sequence with an ATG initiation codon (shown underlined) for proper initiation of translation (Kozak, M. (1987). An Analysis of 5′-Noncoding Sequences from 699 Vertebrate Messenger RNAs. Nucleic Acids Res. 15, 8125-8148; Kozak, M. (1991). An Analysis of Vertebrate mRNA Sequences: Intimations of Translational Control. J. Cell Biology 115, 887-903; Kozak, M. (1990). Downstream Secondary Structure Facilitates Recognition of Initiator Codons by Eukaryotic Ribosomes. Proc. Natl. Acad. Sci. USA 87, 8301-830).

The bla(M) gene contains a TAG stop codon and may be used with the Tag-On-Demand™ System to facilitate expression of a C-terminally-tagged protein, if desired (see previous page for more information).

Note: The C-terminal tag must be provided by the destination vector in the LR recombination reaction.

Shaded regions correspond to DNA sequences transferred from the pENTR/GeneBLAzer™ entry clone into the destination vector following recombination.

Performing the LR Recombination Reaction

Introduction

This section provides guidelines and protocols to perform an LR recombination reaction, transform the reaction mixture into a suitable E. coli host (see below), and to select for an expression clone.

E. coli Host

You may use any recA, endA E. coli strain including TOP10, DH5a™, or equivalent for transformation. Do not transform the LR reaction mixture into E. coli strains that contain the F′ episome (e.g. TOP10F′). These strains contain the ccdA gene and will prevent negative selection of your ccdB-containing destination vector.

Materials Needed

You should have the following materials on hand before beginning:

pENTR/GeneBLAzer™ entry clone (resuspended to 100 ng/μl)

Destination vector of choice (150 ng/μl in TE, pH 8.0)

LR Clonase™ enzyme mix (Invitrogen, Catalog no. 11791-019; keep at −80° C. until immediately before use)

5× LR Clonase™ Reaction Buffer (supplied with the LR Clonase™ enzyme mix)

TE Buffer, pH 8.0 (10 mM Tris-HCl, pH 8.0, 1 mM EDTA)

2 μg/μl Proteinase K solution (supplied with the LR Clonase™ enzyme mix; thaw and keep on ice until use)

Appropriate competent E. coli host and growth media for expression

S.O.C. Medium

LB agar plates containing the appropriate antibiotic to select for expression clones

Introduction

Add the following components to 1.5 ml microcentrifuge tubes at room temperature and mix.

Note: To include a negative control, set up a second sample reaction and substitute TE Buffer, pH 8.0 for the LR Clonase™ enzyme mix (see Step 4).

TABLE 29
Reaction

| Component | Sample |
|---|---|
| pENTR/GeneBLAzer ™ entry | 2 μl |
| clone (100 ng/μl) | |
| Destination vector (150 ng/μl) | 2 μl |
| 5X LR Clonase ™ Reaction Buffer | 4 μl |
| TE Buffer, pH 8.0 | 8 μl |

Remove the LR Clonase™ enzyme mix from −80° C. and thaw on ice (˜2 minutes).

Vortex the LR Clonase™ enzyme mix briefly twice (2 seconds each time).

To each sample above, add 4 μl of LR Clonase™ enzyme mix. Mix well by pipetting up and down.

Reminder: Return LR Clonase™ enzyme mix to −80° C. immediately after use.

Incubate reactions at 25° C. for 1 hour.

Note: Extending the incubation time to 18 hours typically yields more colonies.

Add 2 μl of the Proteinase K solution to each reaction. Incubate for 10 minutes at 37° C.

Transform 1 μl of the LR recombination reaction into a suitable E. coli host (follow the manufacturer's instructions) and select for expression clones.

Note: You may store the LR reaction at −20° C. for up to 1 week before transformation, if desired.

What You Should See

If you use E. coli cells with a transformation efficiency of 1×10⁸ cfu/μg, the LR reaction should give >5000 colonies if the entire LR reaction is transformed and plated.

You may sequence your expression clone to confirm the presence of the bla(M) gene, if desired. If your expression clone contains an ampicillin resistance gene, do not use primers that hybridize within the bla(M) gene as they will also hybridize within the ampicillin resistance gene, contaminating your results.

Transfecting Cells

Introduction

This section provides general information for transfecting your expression clone into the mammalian cell line of choice. We recommend that you include a mock transfection (negative control) in your experiments to help you evaluate your results.

If you plan to detect β-lactamase activity in vivo using the GeneBLAzer™ In Vivo Detection Kit, note that a number of factors including cell type and cell density can influence the degree of the fluorescence signal detected. We recommend taking these factors into account when designing your transfection experiment. For more information, refer to the section entitled General Guidelines to Use the GeneBLAzer™ In Vivo Detection Kit in the GeneBLAzer™ Detection Kits manual.

Methods of Transfection

For established cell lines (e.g. HeLa), consult original references or the supplier of your cell line for the optimal method of transfection. We recommend that you follow exactly the protocol for your cell line. Pay particular attention to medium requirements, when to pass the cells, and at what dilution to split the cells. Further information is provided in Current Protocols in Molecular Biology (Ausubel, F. M., Brent, R., Kingston, R. E., Moore, D. D., Seidman, J. G., Smith, J. A., and Struhl, K. (1994). Current Protocols in Molecular Biology (New York: Greene Publishing Associates and Wiley-Interscience)).

Methods for transfection include calcium phosphate (Chen, C., and Okayama, H. (1987). High-Efficiency Transformation of Mammalian Cells by Plasmid DNA. Mol. Cell. Biol. 7, 2745-2752; Wigler, M., Silverstein, S., Lee, L.-S., Pellicer, A., Cheng, Y.-C., and Axel, R. (1977). Transfer of Purified Herpes Virus Thymidine Kinase Gene to Cultured Mouse Cells. Cell 11, 223-232), lipid-mediated (Felgner, P. L., Holm, M., and Chan, H. (1989). Cationic Liposome Mediated Transfection. Proc. West. Pharmacol. Soc. 32, 115-121; Felgner, P. L. a., and Ringold, G. M. (1989). Cationic Liposome-Mediated Transfection. Nature 337, 387-388) and electroporation (Chu, G., Hayakawa, H., and Berg, P. (1987). Electroporation for the Efficient Transfection of Mammalian Cells with DNA. Nucleic Acids Res. 15, 1311-1326; Shigekawa, K., and Dower, W. J. (1988). Electroporation of Eukaryotes and Prokaryotes: A General Approach to the Introduction of Macromolecules into Cells. BioTechniques 6, 742-751). For high efficiency transfection in a broad range of mammalian cell lines, we recommend using Lipofectamine™ 2000 Reagent (Catalog no. 11668-027) available from Invitrogen.

Detecting β-Lactamase Activity

Introduction

To use the GeneBLAzer™ In Vivo Detection Kit or the GeneBLAzer™ In Vitro Detection Kit to detect 0 lactamase activity, refer to the GeneBLAzer™ Detection Kits manual for detailed information and protocols. A brief description of each detection method is provided below.

In Vitro Detection

Using the GeneBLAzer™ In Vitro Detection Kit allows you to quantitate the amount of intracellular β-lactamase in cells based on the β-lactamase activity in lysates.

To detect β-lactamase activity in mammalian cell lysates, you will use the CCF2-FA substrate. CCF2-FA is the non-esterified, free acid form of CCF2, and is recommended for in vitro use because it is readily soluble in aqueous solution and may be added directly to pre-made cell lysates. Once added to cell lysates, you may quantitate the CCF2-FA fluorescence signal using a fluorescence plate reader or a fluorometer.

To prepare cell lysates from mammalian cells containing the bla(M) gene, you must use a method that will preserve the activity of the β-lactamase enzyme. Refer to the GeneBLAzer™ Detection Kits manual for detailed guidelines and protocols.

In Vivo Detection

Using the GeneBLAzer™ In Vivo Detection Kit allows you to measure β-lactamase activity in live mammalian cells. Once β-lactamase activity has been measured, cells may be cultured further for use in additional assays or other downstream applications.

To detect β-lactamase activity in live mammalian cells, you will use the CCF2-AM substrate. CCF2-AM is the membrane-permeable, esterified form of CCF2, and is recommended for in vivo use because it is non-toxic, lipophilic, and readily enters the cell. Once cells are “loaded” with CCF2-AM, you may quantitate the CCF2 fluorescence signal using a variety of methods.

Features of pENTR/GeneBLAzer™

TABLE 30
Features of pENTR/GeneBLAzer

| Feature | Benefit |
|---|---|
| rrnB T1 and T2 | Prevents basal expression of the β- |
| transcription termination | lactamase gene in E. coli (Orosz, A., |
| sequences | Boros, I., and Venetianer, P. (1991). |
| | Analysis of the Complex |
| | Transcription Termination Region of |
| | the Escherichia coli rrnB Gene. Eur. |
| | J. Biochem. 201, 653-659) |
| attL1 and attL2 sites | Allows site-specific recombination of |
| | the entry clone with a Gateway ® |
| | destination vector (Landy, A. (1989). |
| | Dynamic, Structural, and Regulatory |
| | Aspects of Lambda Site-specific |
| | Recombination. Annu. Rev. Biochem. |
| | 58, 913-949) |
| β-lactamase bla(M) gene | Allows in vivo or in vitro detection of |
| | gene expression (Zlokarnik, G., |
| | Negulescu, P. A., Knapp, T. E., Mere, L., |
| | Burres, N., Feng, L., Whitney, M., |
| | Roemer, K., and Tsien, R. Y. (1998). |
| | Quantitation of Transcription and |
| | Clonal Selection of Single Living |
| | Cells with b-Lactamase as Reporter. |
| | Science 279, 84-88) |
| Kanamycin resistance | Allows selection of the plasmid in E. coli |
| gene | |
| pUC origin | Allows high-copy number replication |
| | and growth in E. coli |

### Nucleotide Sequence Tables

TABLE 31
Nucleotide Sequence of pGeneBLAzer-TOPO ®
(See FIG. 7) (SEQ ID NO: 153)

GACGGATCGGGAGATCTAATACGACTCACTATAGGGAGACCCAAGCTGGC
TAGCGTTTAAACTTAAGCTTGGTACCGAGCTCGGATCCACTAGTCCAGTG
TGGTGGAATTGCCCTTAAGGGCAATTCGCCCTTCACCATGGACCCAGAAA
CGCTGGTGAAAGTAAAAGATGCTGAAGATCAGTTGGGTGCCCGAGTGGGT
TACATCGAACTGGATCTCAACAGCGGTAAGATCCTTGAGAGTTTTCGCCC
CGAAGAACGTTTTCCAATGATGAGCACTTTTAAAGTTCTGCTATGTGGCG
CGGTATTATCCCGTATTGACGCCGGGCAAGAGCAACTCGGTCGCCGCATA
CACTATTCTCAGAATGACTTGGTTGAGTACTCACCAGTGACAGAAAAGCA
TCTTACGGATGGCATGACAGTAAGAGAATTATGCAGTGCTGCCATAACCA
TGAGTGATAACACTGCGGCCAACTTACTTCTGACAACGATCGGAGGACCG
AAGGAGCTAACCGCTTTTTTGCACAACATGGGGGATCATGTAACTCGCCT
TGATCGTTGGGAACCGGAGCTGAATGAAGCCATACCAAACGACGAGCGTG
ACACCACGATGCCTGTAGCAATGGCAACAACGTTGCGCAAACTATTAACT
GGCGAACTACTTACTCTAGCTTCCCGGCAACAATTAATAGACTGGATGGA
GGCGGATAAAGTTGCAGGACCACTTCTGCGCTCGGCCCTTCCGGCTGGCT
GGTTTATTGGTGATAAATCTGGAGCCGGTGAGCGTGGGTCTCGCGGTATC
ATTGCAGCACTGGGGCCAGATGGTAAGCCCTCCCGTATCGTAGTTATCTA
CACGACGGGGAGTCAGGCAACTATGGATGAACGAAATAGACAGATCGCTG
AGATAGGTGCCTCACTGATTAAGCATTGGTAAGATAAACGGGGGAGGCTA
ACTGAAACACGGAAGGAGACAATACCGGAAGGAACCCGCGCTATGACGGC
AATAAAAAGACAGAATAAAACGCACGGGTGTTGGGTCGTTTGTTCATAAA
CGCGGGGTTCGGTCCCAGGGCTGGCACTCTGTCGATACCCCACCGAGACC
CCATTGGGGCCAATACGCCCGCGTTTCTTCCTTTTCCCCACCCCACCCCC
CAAGTTCGGGTGAAGGCCCAGGGCTCGCAGCCAACGTCGGGGCGGCAGGC
CCTGCCATAGCAGATCTGCGCAGCTGGGGCTCTAGGGGGTATCCCCACGC
GCCCTGTAGCGGCGCATTAAGCGCGGCGGGTGTGGTGGTTACGCGCAGCG
TGACCGCTACACTTGCCAGCGGCCTAGCGCCCGCTCCTTTGGCTTTCTTG
CCTTCCTTTGTCGCCACGTTGGCCGGCTTTCCCCGTCAAGCTCTAAATCG
GGGCATCCCTTTAGGGTTCCGATTTAGTGCTTTACGGCACCTCGACCCCA
AAAAACTTGATTAGGGTGATGGTTCACGTAGTGGGCCATCGCCCTGATAG
ACGGTTTTTCGCCCTTTGACGTTGGAGTCCACGTTCTTTAATAGTGGACT
CTTGTTCCAAACTGGAACAACACTCAACCCTATCTCGGTCTATTCTTTTG
ATTTATAAGGGATTTTGGGGATTTCGGCCTATTGGTTAAAAAATGAGCTG
ATTTAACAAAAATTTAACGCGAATTAATTCTGTGGAATGTGTGTCAGTTA
GGGTGTGGAAAGTCCCCAGGCTCCCCAGCAGGCAGAAGTATGCAAAGCAT
GCATCTCAATTAGTCAGCAACCAGGTGTGGAAAGTCCCCAGGCTCCCCAG
CAGGCAGAAGTATGCAAAGCATGCATCTCAATTAGTCAGCAACCATAGTC
CCGCCCCTAACTCCGCCCATCCCGCCCCTAACTCCGCCCAGTTCCGCCCA
TTCTCCGCCCCATGGCTGACTAATTTTTTTTATTTATGCAGAGGCCGAGG
CCGCCTCTGCCTCTGAGCTATTCCAGAAGTAGTGAGGAGGCTTTTTTGGA
GGCCTAGGCTTTTGCAAAAAGCTCCCGGGAGCTTGTATATCCATTTTCGG
ATCTGATCAAGAGACAGGATGAGGATCGTTTCGCATGATTGAACAAGATG
GATTGCACGCAGGTTCTCCGGCCGCTTGGGTGGAGAGGCTATTCGGCTAT
GACTGGGCACAACAGACAATCGGCTGCTCTGATGCCGCCGTGTTCCGGCT
GTCAGCGCAGGGGCGCCCGGTTCTTTTTGTCAAGACCGACCTGTGCGGTG
CCCTGAATGAACTGCAGGACGAGGCAGCGCGGCTATCGTGGCTGGCCACG
ACGGGCGTTCCTTGCGCAGCTGTGCTCGACGTTGTCACTGAAGCGGGAAG
GGACTGGCTGCTATTGGGCGAAGTGCCGGGGCAGGATCTCCTGTCATCTC
ACCTTGCTCCTGCCGAGAAAGTATCCATCATGGCTGATGCAATGCGGCGG
CTGCATACGCTTGATCCGGCTACCTGCCCATTCGACCACCAAGCGAAACA
TCGCATCGAGCGAGCACGTACTCGGATGGAAGCCGGTCTTGTCGATCAGG
ATGATCTGGACGAAGAGCATCAGGGGCTCGCGCGAGCCGAACTGTTCGCC
AGGCTCAAGGCGCGCATGCCCGACGGCGAGGATCTCGTCGTGACCCATGG
CGATGCCTGCTTGCCGAATATCATGGTGGAAAATGGCCGCTTTTCTGGAT
TCATCGACTGTGGCCGGCTGGGTGTGGCGGACCGCTATCAGGACATAGCG
TTGGCTACCCGTGATATTGCTGAAGAGCTTGGCGGCGAATGGGCTGACCG
CTTCCTCGTGCTTTACGGTATCGCCGCTCCCGATTCGCAGCGCATCGCCT
TCTATCGCCTTCTTGACGAGTTCTTCTGAGCGGGACTCTGGGGTTCGAAA
TGACCGACCAAGCGACGCCCAACCTGCCATCACGAGATTTCGATTCCACC
GCCGCCTTCTATGAAAGGTTGGGCTTCGGAATCGTTTTCCGGGACGCCGG
CTGGATGATCCTCCAGCGCGGGGATCTCATGCTGGAGTTCTTCGCGGACC
CCAACTTGTTTATTGCAGCTTATAATGGTTACAAATAAAGCAATAGCATC
ACAAATTTCACAAATAAAGCATTTTTTTCACTGCATTCTAGTTGTGGTTT
GTCCAAACTCATCAATGTATCTTATCATGTCTGTATACCGTCGACCTCTA
GCTAGAGCTTGGCGTAATCATGGTCATAGCTGTTTCCTGTGTGAAATTGT
TATCCGCTCACAATTCCACACAACATACGAGCCGGAAGCATAAAGTGTAA
AGCCTGGGGTGCCTAATGAGTGAGCTAACTCACATTAATTGCGTTGCGCT
CACTGCCCGCTTTCCAGTCGGGAAACCTGTCGTGCCAGCTGCATTAATGA
ATCGGCGAACGCGCGGGGAGAGGCGGTTTGCGTATTGGGCGCTCTTCCGC
TTCCTCGGTCACTGACTCGCTGCGCTCGGTCGTTCGGCTGCGGCGAGCGG
TATCAGCTCACTCAAAGGCGGTAATACGGTTATCCACAGAATCAGGGGAT
AACGCAGGAAAGAACATGTGAGCAAAAGGCCAGCAAAAGGCCAGGAACCG
TAAAAAGGCCGCGTTGCTGGCGTTTTTCCATAGGCTCCGCCCCCCTGACG
AGCATCACAAAAATCGAGGCTCAAGTCAGAGGTGGCGAAACCCGACAGGA
CTATAAAGATACGAGGCGTTTCCCCCTGGAAGCTCCCTCGTGCGCTCTCC
TGTTCCGACCCTGCCGCTTACCGGATACCTGTCCGCCTTTCTCCCTTCGG
GAAGCGTGGCGCTTTCTCAATGCTCACGCTGTAGGTATCTCAGTTCGGTG
TAGGTCGTTCGCTCCAAGCTGGGCTGTGTGCACGAACCCCCCGTTCAGCC
CGACCGCTGCGCCTTATCCGGTAACTATCGTCTTGAGTCCAACCCGGTAA
GACACGACTTATCGCGACTGGCAGCAGCCACTGGTAACAGGATTAGCAGA
GCGAGGTATGTAGGCGGTGCTACAGAGTTCTTGAAGTGGTGGCGTAACTA
CGGCTACACTAGAAGGACAGTATTTGGTATCTGCGCTCTGCTGAAGCCAG
TTACCTTCGGAAAAAGAGTTGGTAGCTCTTGATCCGGCAAACAAACCACC
GCTGGTAGCGGTGGTTTTTTTGTTTGCAAGCAGCAGATTACGCGCAGAAA
AAAAGGATCTCAAGAAGATCCTTTGATCTTTTCTACGGGGTCTGACGCTC
AGTGGAACGAAAACTCACGTTAAGGGATTTTGGTCATGAGATTATCAAAA
AGGATCTTCACCTAGATCCTTTTAAATTAAAAATGAAGTTTTAAATCAAT
CTAAAGTATATATGAGTAAACTTGGTCTGACAGTTACCAATGCTTAATCA
GTGAGGCACCTATCTCAGCGATCTGTCTATTTGGTTCATCCATAGTTGCC
TGACTCCCCGTCGTGTAGATAACTACGATACGGGAGGGCTTACCATCTGG
CCCCAGTGCTGCAATGATACCGCGAGACCGACGCTCACCGGCTCCAGATT
TATCAGCAATAAACCAGCCAGCCGGAAGGGCCGAGCGCAGAAGTGGTCCT
GCAACTTTATCCGCCTCCATCCAGTCTATTAATTGTTGCCGGGAAGCTAG
AGTAAGTAGTTCGCCAGTTAATAGTTTGCGCAACGTTGTTGCCATTGCTA
CAGGCATCGTGGTGTCACGCTCGTCGTTTGGTATGGCTTCATTCAGCTCC
GGTTCCCAACGATCAAGGCGAGTTACATGATCCCCCATGTTGTGCAAAAA
AGCGGTTAGCTCCTTCGGTCCTCCGATCGTTGTCAGAAGTAAGTTGGCCG
CAGTGTTATCACTCATGGTTATGGCAGCACTGCATAATTCTCTTACTGTC
ATGCCATCCGTAAGATGCTTTTCTGTGACTGGTGAGTACTCAACCAAGTC
ATTCTGAGAATAGTGTATGCGGCGACCGAGTTGCTCTTGCCCGGCGTCAA
TACGGGATAATACCGCGCCACATAGCAGAACTTTAAAAGTGCTCATCATT
GGAAAACGTTCTTCGGGGCGAAAACTCTCAAGGATCTTACCGCTGTTGAG
ATCCAGTTCGATGTAACCCACTCGTGCACCCAACTGATCTTCAGCATCTT
TTACTTTCACCAGCGTTTCTGGGTGAGCAAAAACAGGAAGGCAAAATGCC
GCAAAAAAGGGAATAAGGGCGACACGGAAATGTTGAATACTCATACTCTT
CCTTTTTCAATATTATTGAAGCATTTATCAGGGTTATTGTCTCATGAGCG
GATACATATTTGAATGTATTTAGAAAAATAAACAAATAGGGGTTCCGCGC
ACATTTCCCCGAAAAGTGCCACCTGACGTC

TABLE 32
Nucleotide Sequence of
pcDNA ™6.2/cGeneBLAzer ™-DEST (See FIG. 13)
(SEQ ID NO:154)

GACGGATCGGGAGATCTCCCGATCCCCTATGGTGGACTCTGAGTACAATC
TGCTCTGATGCCGCATAGTTAAGCCAGTATCTGCTCCCTGCTTGTGTGTT
GGAGGTCGCTGAGTAGTGCGCGAGCAAAATTTAAGCTACAACAAGGCAAG
GCTTGACCGACAATTGCATGAAGAATCTGCTTAGGGTTAGGCGTTTTGCG
CTGCTTCGCGATGTACGGGCCAGATATACGCGTTGACATTGATTATTGAC
TAGTTATTAATAGTAATCAATTACGGGGTCATTAGTTCATAGCCCATATA
TGGAGTTCCGCGTTACATAACTTACGGTAAATGGCCCGCCTGGCTGACCG
CCCAACGACCCCCGCCCATTGACGTCAATAATGACGTATGTTCCCATAGT
AACGCCAATAGGGACTTTCCATTGACGTCAATGGGTGGAGTATTTACGGT
AAACTGCCCACTTGGCAGTACATCAAGTGTATCATATGCCAAGTACGCCC
CCTATTGACGTCAATGACGGTAAATGGCCCGCCTGGCATTATGCCCAGTA
CATGACCTTATGGGACTTTCCTACTTGGCAGTACATCTACGTATTAGTCA
TCGCTATTACCATGGTGATGCGGTTTTGGCAGTACATCAATGGGCGTGGA
TAGCGGTTTGACTCACGGGGATTTCCAAGTCTCCACCCCATTGACGTCAA
TGGGAGTTTGTTTTGGCACCAAAATCAACGGGACTTTCCAAAATGTCGTA
ACAACTCCGCCCCATTGACGCAAATGGGCGGTAGGCGTGTACGGTGGGAG
GTCTATATAAGCAGAGCTCTCTGGCTAACTAGAGAAGCCAGTGCTTACTG
GCTTATCGAAATTAATACGACTCACTATAGGGAGACCCAAGCTGGCTAGT
TAAGCTGAGGATCAACAAGTTTGTACAAAAAAGGTGAACGAGAAACGTAA
AATGATATAAATATCAATATATTAAATTAGATTTTGCATAAAAAACAGAC
TACATAATACTGTAAAACACAACATATCCAGTCACTATGGCGGCCGCATT
AGGCACCCCAGGCTTTACACTTTATGCTTCCGGCTCGTATAATGTGTGGA
TTTTGAGTTAGGATCCGTCGAGATTTTCAGGAGCTAAGGAAGCTAAAATG
GAGAAAAAAATCACTGGATATACCACCGTTGATATATCCCAATGGCATCG
TAAAGAACATTTTGAGGCATTTCAGTCAGTTGCTCAATGTACCTATAACC
AGACCGTTCAGGTGGATATTACGGCCTTTTTAAAGACCGTAAAGAAAAAT
AAGCACAAGTTTTATCCGGCCTTTATTCACATTCTTGCCCGCCTGATGAA
TGCTCATCCGGAATTGGGTATGGCAATGAAAGACGGTGAGCTGGTGATAT
GGGATAGTGTTCAGCCTTGTTACACCGTTTTCCATGAGCAAACTGAAACG
TTTTCATCGCTCTGGAGTGAATACCACGACGATTTCCGGCAGTTTCTACA
CATATATTCGCAAGATGTGGCGTGTTACGGTGAAAACCTGGCCTATTTCC
CTAAAGGGTTTATTGAGAATATGTTTTTCGTCTCAGCCAATCCCTGGGTG
AGTTTCACCAGTTTTGATTTAAACGTGGCCAATATGGACAACTTCTTCGC
CCCCGTTTTCACCATGGGCAAATATTATACGCAAGGCGACAAGGTGCTGA
TGCCGCTGGCGATTCAGGTTCATCATGCCGTTTGTGATGGCTTCCATGTC
GGCAGAATGCTTAATGAATTACAACAGTACTGCGATGAGTGGCAGGGCGG
GGCGTAAAGATCTGGATCCGGCTTACTAAAAGCCAGATAACAGTATGCGT
ATTTGCGCGCTGATTTTTGCGGTATAAGAATATATACTGATATGTATACC
CGAAGTATGTCAAAAAGAGGTATGCTATGAAGCAGCGTATTACAGTGACA
GTTGACAGCGACAGCTATCAGTTGCTCAAGGCATATATGATGTCAATATC
TCCGGTCTGGTAAGCACAACCATGCAGAATGAAGCCCGTCGTCTGCGTGC
CGAACGCTGGAAAGCGGAAAATCAGGAAGGGATGGCTGAGGTCGCCCGGT
TTATTGAAATGAACGGCTCTTTTGCTGACGAGAACAGGGGCTGGTGAAAT
GCAGTTTAAGGTTTACACCTATAAAAGAGAGAGCCGTTATCGTCTGTTTG
TGGATGTACAGAGTGATATTATTGACACGCCCGGGCGACGGATGGTGATC
CCCCTGGCCAGTGCACGTCTGCTGTCAGATAAAGTCTCCCGTGAACTTTA
CCCGGTGGTGCATATCGGGGATGAAAGCTGGCGCATGATGACCACCGATA
TGGCCAGTGTGCCGGTCTCCGTTATCGGGGAAGAAGTGGCTGATCTCAGC
CACCGCGAAAATGACATCAAAAACGCCATTAACCTGATGTTCTGGGGAAT
ATAAATGTCAGGCTCCCTTATACACAGCCAGTCTGCAGGTCGACCATAGT
GACTGGATATGTTGTGTTTTACAGTATTATGTAGTCTGTTTTTTATGCAA
AATCTAATTTAATATATTGATATTTATATCATTTTACGTTTCTCGTTCAG
CTTTCTTGTACAAAGTGGTTGATGCTGTTATGGACCCAGAAACGCTGGTG
AAAGTAAAAGATGCTGAAGATCAGTTGGGTGCACGAGTGGGTTACATCGA
ACTGGATCTCAACAGCGGTAAGATCCTTGAGAGTTTTCGCCCCGAAGAAC
GTTTTCCAATGATGAGCACTTTTAAAGTTCTGCTATGTGGCGCGGTATTA
TCCCGTATTGACGCCGGGCAAGAGCAACTCGGTCGCCGCATACACTATTC
TCAGAATGACTTGGTTGAGTACTCACCAGTCACAGAAAAGCATCTTACGG
ATGGCATGACAGTAAGAGAATTATGCAGTGCTGCCATAACCATGAGTGAT
AACACTGCGGCCAACTTACTTCTGACAACGATCGGAGGACCGAAGGAGCT
AACCGCTTTTTTGCACAACATGGGGGATCATGTAACTCGCCTTGATCGTT
GGGAACCGGAGCTGAATGAAGCCATACCAAACGACGAGCGTGACACCACG
ATGCCTGTAGCAATGGCAACAACGTTGCGCAAACTATTAACTGGCGAACT
ACTTAGTCTAGCTTCCCGGCAACAATTAATAGACTGGATGGAGGCGGATA
AAGTTGCAGGACCACTTCTGCGGTCGGCCCTTCCGGCTGGCTGGTTTATT
GCTGATAAATCTGGAGCCGGTGAGCGTGGGTCTCGCGGTATCATTGCAGC
ACTGGGGCCAGATGGTAAGCCCTCCCGTATCGTAGTTATCTACACGACGG
GGAGTCAGGCAACTATGGATGAACGAAATAGACAGATCGCTGAGATAGGT
GCCTCACTGATTAAGCATTGGTAACCGGTTAGTAATGAGTTTAAACGGGG
GAGGCTAACTGAAACACGGAAGGAGACAATACCGGAAGGAACCCGCGCTA
TGACGGCAATAAAAAGAGAGAATAAAACGCACGGGTGTTGGGTCGTTTGT
TCATAAACGcGGGGTTCGGTCCCAGGGCTGGCACTCTGTCGATACCCCAC
CGAGACCCCATTGGGGCCAATAGGCCCGCGTTTCTTCCTTTTCCCCACCC
CACCCCCGAAGTTCGGGTGAAGGCCCAGGGCTCGCAGCCAACGTCGGGGC
GGCAGGCCCTGCCATAGCAGATCTGCGCAGCTGGGGCTCTAGGGGGTATC
CCGACGCGCCCTGTAGCGGCGCATTAAGCGCGGCGGGTGTGGTGGTTACG
CGCAGCGTGACCGCTACACTTGCCAGCGCCCTAGCGCCCGCTCCTTTCGC
TTTCTTCCCTTCCTTTCTCGCCACGTTCGCCGGCTTTCCCCGTCAAGCTC
TAAATCGGGGCATCCCTTTAGGGTTCCGATTTAGTGCTTTACGGCACCTC
GACCCCAAAAAACTTGATTAGGGTGATGGTTCACGTAGTGGGCCATCGCC
CTGATAGACGGTTTTTCGCCCTTTGACGTTGGAGTCCACGTTCTTTAATA
GTGGACTCTTGTTCCAAACTGGAACAACACTCAACCCTATCTCGGTCTAT
TCTTTTGATTTATAAGGGATTTTGGGGATTTCGGCCTATTGGTTAAAAAA
TGAGCTGATTTAACAAAAATTTAACGCGAATTAATTCTGTGGAATGTGTG
TCAGTTAGGGTGTGGAAAGTCCCCAGGCTCCCCAGCAGGCAGAAGTATGC
AAAGCATGCATCTCAATTAGTCAGCAACCAGGTGTGGAAAGTCCCCAGGC
TCCCCAGCAGGCAGAAGTATGCAAAGCATGCATCTCAATTAGTCAGCAAC
CATAGTCCCGCCCCTAACTGCGCCCATCCCGCCCCTAACTCCGCCCAGTT
CCGCCCATTCTCCGCCCCATGGCTGACTAATTTTTTTTATTTATGCAGAG
GCCGAGGCCGCCTCTGCCTCTGAGCTATTCCAGAAGTAGTGAGGAGGCTT
TTTTGGAGGCCTAGGCTTTTGCAAAAAGCTCCCGGGAGCTTGTATATCCA
TTTTCGGATCTGATCAGCACGTGTTGACAATTAATCATCGGCATAGTATA
TCGGCATAGTATAATACGACAAGGTGAGGAACTAAACCATGGCCAAGCCT
TTGTCTCAAGAAGAATCCACCCTCATTGAAAGAGCAACGGCTACAATCAA
CAGCATCCCCATCTCTGAAGACTACAGCGTCGCCAGCGCAGCTCTCTCTA
GCGACGGCGGCATCTTCACTGGTGTCAATGTATATCATTTTACTGGGGGA
CCTTGTGCAGAACTCGTGGTGCTGGGCACTGCTGCTGCTGCGGCAGCTGG
CAACCTGACTTGTATCGTCGCGATCGGAAATGAGAACAGGGGCATCTTGA
GCCCCTGCGGACGGTGCCGACAGGTGCTTCTCGATCTGCATCCTGGGATC
AAAGCCATAGTGAAGGACAGTGATGGACAGCCGACGGCAGTTGGGATTCG
TGAATTGCTGCCCTCTGGTTATGTGTGGGAGGGCTAAGCACTTCGTGGCC
GAGGAGCAGGACTGACACGTGCTACGAGATTTCGATTCCACCGCCGCCTT
CTATGAAAGGTTGGGCTTCGGAATCGTTTTCCGGGACGCCGGCTGGATGA
TCCTCCAGCGCGGGGATCTCATGCTGGAGTTCTTCGCCCACCCCAACTTG
TTTATTGCAGCTTATAATGGTTACAAATAAAGCAATAGCATCACAAATTT
CACAAATAAAGCATTTTTTTCACTGCATTCTAGTTGTGGTTTGTCCAAAC
TCATCAATGTATCTTATCATGTCTGTATACCGTCGACCTCTAGCTAGAGC
TTGGCGTAATCATGGTCATAGCTGTTTCCTGTGTGAAATTGTTATCCGCT
CACAATTCCACACAACATACGAGCCGGAAGCATAAAGTGTAAAGCCTGGG
GTGCCTAATGAGTGAGCTAACTCACATTAATTGCGTTGCGCTCACTGCCC
GCTTTCCAGTCGGGAAACCTGTCGTGCCAGCTGCATTAATGAATCGGCCA
ACGCGCGGGGAGAGGCGGTTTGCGTATTGGGCGCTCTTCCGCTTCCTCGC
TCACTGACTCGCTGCGCTCGGTCGTTCGGCTGCGGCGAGCGGTATCAGCT
CACTCAAAGGCGGTAATACGGTTATCCACAGAATCAGGGGATAACGCAGG
AAAGAACATGTGAGCAAAAGGCCAGCAAAAGGCCAGGAACCGTAAAAAGG
CCGCGTTGCTGGCGTTTTTGCATAGGCTCCGCCCCCCTGACGAGGATCAC
AAAAATCGACGCTCAAGTCAGAGGTGGCGAAACCCGACAGGACTATAAAG
ATACCAGGCGTTTCCCCCTGGAAGCTCCCTCGTGCGCTCTCCTGTTCCGA
CCCTGGGGCTTAGCGGATACCTGTCCGCGTTTCTCCCTTCGGGAAGCGTG
GCGCTTTCTCATAGCTCACGCTGTAGGTATCTCAGTTCGGTGTAGGTCGT
TCGCTCCAAGCTGGGCTGTGTGCACGAACCCCCCGTTGAGCCCGACCGCT
GCGGCTTATCCGGTAACTATCGTCTTGAGTCCAACCCGGTAAGACACGAC
TTATCGCCACTGGCAGGAGCCACTGGTAAGAGGATTAGCAGAGCGAGGTA
TGTAGGCGGTGCTACAGAGTTCTTGAAGTGGTGGCCTAACTACGGCTACA
CTAGAAGAACAGTATTTGGTATCTGCGCTCTGCTGAAGCCAGTTAGCTTC
GGAAAAAGAGTTGGTAGCTCTTGATCCGGCAAACAAAGCACCGCTGGTAG
CGGTTTTTTTGTTTGCAAGCAGCAGATTACGCGCAGAAAAAAAGGATCTC
AAGAAGATCCTTTGATCTTTTCTACGGGGTCTGACGCTCAGTGGACGAAA
ACTCACGTTAAGGGATTTTGGTCATGAGATTATCAAAAAGGATCTTCACC
TAGATCCTTTTAAATTAAAAATGAAGTTTTAAATCAATCTAAAGTATATA
TGAGTAAACTTGGTCTGACAGTTACCAATGCTTAATCAGTGAGGCACCTA
TCTCAGCGATCTGTCTATTTCGTTCATCCATAGTTGCCTGACTCCCCGTC
GTGTAGATAAGTACGATACGGGAGGGCTTACCATCTGGCCCCAGTGCTGC
AATGATACCGCGAGACCCACGCTCACCGGCTCCAGATTTATCAGCAATAA
ACCAGCCAGCCGGAAGGGCCGAGCGCAGAAGTGGTCCTGCAACTTTATGC
GCGTCCATCCAGTCTATTAATTGTTGCCGGGAAGCTAGAGTAAGTAGTTC
GCCAGTTAATAGTTGCGCAACGTTGTTGCCATTGCTACAGGCATGGTGGT
GTCACGCTCGTCGTTTGGTATGGCTTCATTCAGCTCCGGTTCCCAACGAT
CAAGGCGAGTTACATGATCCCCCATGTTGTGCAAAAAAGCGGTTAGCTCC
TTCGGTCCTCCGATCGTTGTCAGAAGTAAGTTGGCGGCAGTGTTATCACT
CATGGTTATGGCAGCACTGCATAATTCTCTTACTGTCATGCCATCCGTAA
GATGCTTTTCTGTGACTGGTGAGTACTCAACCAAGTCATTCTGAGAATAG
TGTATGCGGCGACCGAGTTGCTCTTGCCCGGCGTCAATACGGGATAATAC
CGCGCCACATAGCAGAACTTTAAAAGTGCTCATCATTGGAAAACGTTCTT
CGGGGCGAAAACTCTCAAGGATCTTACCGCTGTTGAGATCCAGTTCGATG
TAACCCACTCGTGCACCCAACTGATCTTCAGCATCTTTTACTTTCACCAG
CGTTTCTGGGTGAGCAAAAACAGGAAGGCAAAATGCCGGAAAAAAGGGAA
TAAGGGCGACACGGAAATGTTGAATACTCATACTCTTCCTTTTTGAATAT
TATTGAAGCATTTATCAGGGTTATTGTCTCATGAGCGGATAGATATTTGA
ATGTATTTAGAAAAATAAACAAATAGGGGTTCCGCGCACATTTCCCCGAA
AAGTGCCACCTGACGTC

TABLE 33
Nucleotide Sequence of
pcDNA ™6.2/nGeneBLAzer ™-DEST (See FIG. 14)
(SEQ ID NO: 155)

GACGGATCGGGAGATCTCCCGATCCCCTATGGTGCACTCTCAGTACAATC
TGCTCTGATGCCGCATAGTTAAGCCAGTATCTGCTCCCTGCTTGTGTGTT
GGAGGTCGCTGAGTAGTGCGCGAGCAAAATTTAAGCTACAACAAGGCAAG
GCTTGACCGACAATTGCATGAAGAATCTGCTTAGGGTTAGGCGTTTTGCG
CTGCTTCGCGATGTACGGGCCAGATATACGCGTTGACATTGATTATTGAC
TAGTTATTAATAGTAATCAATTACGGGGTCATTAGTTCATAGCCCATATA
TGGAGTTCCGCGTTACATAACTTACGGTAAATGGCCCGCCTGGCTGACCG
CCCAACGACCCCCGCCCATTGACGTCAATAATGACGTATGTTCCCATAGT
AACGCCAATAGGGACTTTCCATTGACGTCAATGGGTGGAGTATTTACGGT
AAACTGCCCACTTGGCAGTACATCAAGTGTATCATATGCCAAGTACGCCC
CCTATTGACGTCAATGACGGTAAATGGCCCGCCTGGCATTATGCCCAGTA
CATGACCTTATGGGACTTTCCTACTTGGCAGTACATCTACGTATTAGTCA
TCGCTATTACCATGGTGATGCGGTTTTGGCAGTACATCAATGGGCGTGGA
TAGCGGTTTGACTCACGGGGATTTCCAAGTCTCCACCCCATTGACGTCAA
TGGGAGTTTGTTTTGGCACCAAAATCAACGGGACTTTCCAAAATGTCGTA
ACAACTCCGCCCCATTGACGCAAATGGGCGGTAGGCGTGTACGGTGGGAG
GTCTATATAAGCAGAGCTCTCTGGCTAACTAGAGAACCCACTGGTTACTG
GCTTATCGAAATTAATACGACTCACTATAGGGAGACCCAAGGTGGCTAGT
TAAGCTGAGCATCAACAAGTTTGTACAAAAAAGCTGAACGAGAAACGTAA
AATGATATAAATATCAATATATTAAATTAGATTTTGCATAAAAAACAGAC
TACATAATACTGTAAAACACAACATATCCAGTCACTATGGCGGCCGCATT
AGGCACCCCAGGCTTTACACTTTATGCTTCCGGCTCGTATAATGTGTGGA
TTTTGAGTTAGGATCCGTCGAGATTTTCAGGAGCTAAGGAAGCTAAAATG
GAGAAAAAAATCACTGGATATACCACCGTTGATATATCCCAATGGCATCG
TAAAGAACATTTTGAGGCATTTCAGTCAGTTGCTCAATGTACCTATAACC
AGACCGTTCAGCTGGATATTACGGCCTTTTTAAAGACCGTAAAGAAAAAT
AAGCACAAGTTTTATCCGGCCTTTATTCACATTCTTGCCCGCGTGATGAA
TGCTCATCCGGAATTCCGTATGGCAATGAAAGACGGTGAGCTGGTGATAT
GGGATAGTGTTCACCCTTGTTACACCGTTTTCCATGAGCAAACTGAAACG
TTTTGATCGCTCTGGAGTGAATACCACGACGATTTCCGGCAGTTTCTACA
CATATATTCGCAAGATGTGGCGTGTTACGGTGAAAACCTGGCCTATTTCC
CTAAAGGGTTTATTGAGAATATGTTTTTCGTCTCAGCCAATCCCTGGGTG
AGTTTCACCAGTTTTGATTTAAACGTGGCCAATATGGACAACTTCTTCGC
CCCCGTTTTCACCATGGGCAAATATTATACGCAAGGCGACAAGGTGCTGA
TGCCGCTGGCGATTCAGGTTCATCATGCCGTTTGTGATGGCTTCCATGTC
GGCAGAATGCTTAATGAATTACAACAGTACTGCGATGAGTGGCAGGGCGG
GGCGTAAAGATCTGGATCCGGCTTACTAAAAGCCAGATAACAGTATGCGT
ATTTGCGCGCTGATTTTTGCGGTATAAGAATATATACTGATATGTATACC
CGAAGTATGTCAAAAAGAGGTATGCTATGAAGCAGCGTATTACAGTGACA
GTTGACAGCGACAGCTATCAGTTGCTCAAGGCATATATGATGTCAATATC
TCCGGTCTGGTAAGCACAACCATGCAGAATGAAGCCCGTCGTCTGCGTGC
CGAACGCTGGAAAGCGGAAAATCAGGAAGGGATGGCTGAGGTCGCCCGGT
TTATTGAAATGAACGGCTCTTTTGCTGACGAGAACAGGGGCTGGTGAAAT
GCAGTTTAAGGTTTACACCTATAAAAGAGAGAGCCGTTATCGTCTGTTTG
TGGATGTACAGAGTGATATTATTGAGACGCCCGGGCGACGGATGGTGATC
CCCCTGGCCAGTGCACGTCTGCTGTCAGATAAAGTCTCCCGTGAACTTTA
CCCGGTGGTGCATATCGGGGATGAAAGCTGGCGCATGATGACCACCGATA
TGGCCAGTGTGCCGGTCTCCGTTATCGGGGAAGAAGTGGCTGATCTCAGC
CACCGCGAANATGACATCAAAAACGCCATTAACCTGATGTTCTGGGGAAT
ATAAATGTGAGGCTCCCTTATACACAGCCAGTCTGCAGGTCGACCATAGT
GACTGGATATGTTGTGTTTTACAGTATTATGTAGTCTGTTTTTTATGCAA
AATCTAATTTAATATATTGATATTTATATCATTTTACGTTTCTCGTTCAG
CTTTCTTGTACAAAGTGGTTGATGCTGTTATGGACCCAGAAACGCTGGTG
AAAGTAAAAGATGCTGAAGATCAGTTGGGTGCACGAGTGGGTTACATCGA
ACTGGATCTCAACAGCGGTAAGATCCTTGAGAGTTTTCGCCCCGAAGAAC
GTTTTCCAATGATGAGCACTTTTAAAGTTCTGCTATGTGGCGCGGTATTA
TCCCGTATTGACGCCGGGCAAGAGCAACTCGGTCGCCGCATACACTATTC
TCAGAATGACTTGGTTGAGTACTCACCAGTCACAGAAAAGCATCTTACGG
ATGGCATGACAGTAAGAGAATTATGCAGTGCTGCCATAACCATGAGTGAT
AACACTGCGGCCAACTTACTTCTGACAACGATCGGAGGACCGAAGGAGCT
AACCGCTTTTTTGCACAACATGGGGGATCATGTAACTCGCGTTGATCGTT
GGGAACCGGAGCTGAATGAAGCCATACCAAACGACGAGCGTGACACCACG
ATGCCTGTAGCAATGGCAACAACGTTGCGCAAACTATTAACTGGCGAACT
ACTTACTCTAGCTTCCCGGCAACAATTAATAGACTGGATGGAGGCGGATA
AAGTTGCAGGACCACTTCTGCGCTCGGCCCTTCCGGCTGGCTGGTTTATT
GCTGATAAATGTGGAGCCGGTGAGCGTGGGTCTCGCGGTATCATTGCAGC
ACTGGGGCCAGATGGTAAGCCCTCCCGTATCGTAGTTATCTACACGACGG
GGAGTCAGGCAACTATGGATGAACGAAATAGACAGATCGCTGAGATAGGT
GCCTCACTGATTAAGCATTGGTAACCGGTTAGTAATGAGTTTAAACGGGG
GAGGCTAACTGAAACAGGGAAGGAGACAATACCGGAAGGAACCCGCGCTA
TGACGGCAATAAAAAGACAGAATAAAACGCACGGGTGTTGGGTCGTTTGT
TCATAAACGCGGGGTTCGGTCCCAGGGCTGGCACTGTGTCGATACCCCAC
CGAGACCCCATTGGGGCCAATACGCCCGCGTTTCTTCCTTTTCCCCACCC
CACCCCCCAAGTTCGGGTGAAGGCCCAGGGCTCGCAGCCAACGTCGGGGC
GGCAGGCCCTGCCATAGCAGATCTGCGCAGCTGGGGCTCTAGGGGGTATC
CCCACGCGCCCTGTAGCGGCGCATTAAGCGCGGCGGGTGTGGTGGTTACG
CGCAGCGTGACCGCTACACTTGCCAGCGCCCTAGCGCCCGCTCCTTTCGC
TTTCTTCCCTTCCTTTCTCGCCACGTTCGCCGGCTTTCCCCGTCAAGCTC
TAAATCGGGGCATCCCTTTAGGGTTCCGATTTAGTGCTTTACGGCACCTC
GACCCCAAAAAACTTGATTAGGGTGATGGTTCACGTAGTGGGCCATCGCC
CTGATAGACGGTTTTTCGCCCTTTGACGTTGGAGTCCACGTTCTTTAATA
GTGGACTCTTGTTCCAAACTGGAACAACACTCAACCCTATCTCGGTCTAT
TCTTTTGATTTATAAGGGATTTTGGGGATTTCGGCCTATTGGTTAAAAAA
TGAGCTGATTTAACAAAAATTTAACGCGAATTAATTCTGTGGAATGTGTG
TCAGTTAGGGTGTGGAAAGTCCCCAGGCTCCCCAGCAGGCAGAAGTATGC
AAAGCATGCATCTCAATTAGTCAGCAACCAGGTGTGGAAAGTCCCCAGGC
TCCCCAGCAGGCAGAAGTATGCAAAGCATGCATCTCAATTAGTCAGCAAC
CATAGTCCCGCCCCTAACTCCGCCCATCCCGCCCCTAACTCCGCCCAGTT
CCGGCCATTCTCCGCCCCATGGCTGACTAATTTTTTTTATTTATGCAGAG
GCCGAGGCCGCCTCTGCCTCTGAGCTATTCCAGAAGTAGTGAGGAGGCTT
TTTTGGAGGCCTAGGCTTTTGCAAAAAGCTCCCGGGAGCTTGTATATCCA
TTTTCGGATGTGATCAGCACGTGTTGACAATTAATCATCGGCATAGTATA
TCGGCATAGTATAAACGACAAGGTGAGGAACTAAACCATGGCCAAGCCTT
TGTCTCAAGAAGAATCCACCCTCATTGAAAGAGCAACGGCTACAATCAAC
AGCATCCCCATCTCTGAAGACTACAGCGTCGCCAGCGCAGCTCTCTCTAG
CGACGGCCGCATCTTCACTGGTGTCAATGTATATCATTTTACTGGGGGAC
CTTGTGCAGAACTCGTGGTGCTGGGCACTGCTGCTGCTGCGGCAGCTGGC
AACCTGACTTGTATCGTCGCGATCGGAAATGAGAACAGGGGCATCTTGAG
CCCCTGCGGACGGTGCCGACAGGTGCTTCTCGATCTGCATCCTGGGATCA
AAGGCATAGTGAAGGACAGTGATGGACAGCCGACGGCAGTTGGGATTCGT
GAATTGCTGCCCTCTGGTTATGTGTGGGAGGGCTAAGCACTTCGTGGCCG
AGGAGCAGGACTGACACGTGCTAGGAGATTTCATTCCACCGCCGCCTTCT
ATGAAAGGTTGGGCTTCGGAATCGTTTTCCGGGACGCCGGCTGGATGATC
CTCCAGCGCGGGGATCTCATGGTGGAGTTCTTCGCCCACCCCAACTTGTT
TATTGCAGCTTATAATGGTTACAAATAAAGCAATAGCATCACAAATTTCA
CAAATAAAGCATTTTTTTCACTGCATTCTAGTTGTGGTTTGTCCAAACTC
ATCAATGTATCTTATCATGTCTGTATACCGTCGACCTCTAGCTAGAGCTT
GGCGTAATCATGGTCATAGCTGTTTCCTGTGTGAAATTGTTATCCGCTCA
CAATTCCACACAACATACGAGCCGGAAGCATAAAGTGTAAAGCCTGGGGT
GCCTAATGAGTGAGCTAACTCACATTAATTGCGTTGCGCTCACTGCCCGC
TTTCCAGTCGGGAAACCTGTCGTGCCAGCTGCATTAATGAATCGGCCAAC
GCGCGGGGAGAGGCGGTTTGCGTATTGGGCGCTCTTCCGCTTCCTCGCTC
ACTGACTCGCTGCGCTCGGTGGTTCGGCTGCGGCGAGCGGTATCAGGTCA
CTCAAAGGCGGTAATACGGTTATCCACAGAATCAGGGGATAACGCAGGAA
AGAACATGTGAGCAAAAGGCCAGCAAAAGGCCAGGAACCGTAAAAAGGCC
GCGTTGCTGGCGTTTTTCCATAGGCTCCGCCCCCCTGACGAGCATCACAA
AAATCGACGCTCAAGTCAGAGGTGGCGAAACCCGACAGGACTATAAAGAT
ACCAGGCGTTTCCCCGTGGAAGCTCCCTCGTGCGCTCTCCTGTTCCGAGC
CTGCCGCTTACCGGATACCTGTCCGCCTTTCTCCCTTCGGGAAGGGTGGC
GCTTTCTCATAGCTCACGCTGTAGGTATCTCAGTTCGGTGTAGGTCGTTC
GCTCCAAGCTGGGCTGTGTGCACGAACCCCCCGTTCAGCCGGACCGCTGC
GCCTTATCCGGTAACTATCGTCTTGAGTCCAACCCGGTAAGACACGACTT
ATCGCCACTGGCAGCAGCGACTGGTAACAGGATTAGCAGAGCGAGGTATG
TAGGCGGTGCTACAGAGTTCTTGAAGTGGTGGCGTAACTACGGCTACACT
AGAAGAACAGTATTTGGTATCTGCGCTCTGCTGAAGCCAGTTACCTTCGG
AAAAAGAGTTGGTAGCTCTTGATCGGGCAAACAAACCACCGCTGGTAGCG
GTTTTTTTGTTTGCAAGCAGCAGATTACGCGCAGAAAAAAAGGATCTCAA
GAAGATCCTTTGATCTTTTCTACGGGGTCTGACGCTCAGTGGAACGAAAA
CTCACGTTAAGGGATTTTGGTCATGAGATTATCAAAAAGGATCTTCACCT
AGATCCTTTTAAATTAAAAATGAAGTTTTAAATCAATCTAAAGTATATAT
GAGTAAACTTGGTCTGACAGTTACCAATGCTTAATCAGTGAGGCACCTAT
CTCAGCGATCTGTCTATTTCGTTCATCCATAGTTGCCTGACTCCCCGTCG
TGTAGATAACTACGATACGGGAGGGCTTACCATCTGGCCCCAGTGCTGCA
ATGATACCGCGAGACCGACGCTCACCGGCTCGAGATTTATCAGCAATAAA
CCAGGCAGCCGGAAGGGCCGAGCGCAGAAGTGGTCCTGCAACTTTATCCG
CCTCCATCCAGTCTATTAATTGTTGCCGGGAAGCTAGAGTAAGTAGTTCG
CCAGTTAATAGTTTGCGCAACGTTGTTGCCATTGCTACAGGCATCGTGGT
GTCACGCTCGTCGTTTGGTATGGCTTCATTCAGCTCCGGTTCCCAACGAT
CAAGGCGAGTTACATGATCCCCCATGTTGTGCAAAAAAGCGGTTAGCTCC
TTCGGTCCTCCGATCGTTGTCAGAAGTAAGTTGGCCGCAGTGTTATCACT
CATGGTTATGGCAGCACTGCATAATTCTCTTACTGTCATGCCATCCGTAA
GATGCTTTTCTGTGACTGGTGAGTACTCAACCAAGTCATTCTGAGAATAG
TGTATGCGGCGACCGAGTTGCTCTTGCCCGGCGTCAATACGGGATAATAC
CGCGCCACATAGCAGAACTTTAAAAGTGCTCATCATTGGAAAACGTTCTT
CGGGGCGAAAACTCTCAAGGATCTTACCGCTGTTGAGATCCAGTTCGATG
TAACCCACTCGTGCACCCAACTGATCTTCAGCATCTTTTACTTTCACCAG
CGTTTCTGGGTGAGCAAAAACAGGAAGGCAAAATGCCGCAAAAAAGGGAA
TAAGGGCGACACGGAAATGTTGAATACTCATACTCTTCCTTTTTCAATAT
TATTGAAGCATTTATCAGGGTTATTGTCTCATGAGCGGATACATATTTGA
ATGTATTTAGAAAAATAAACAAATAGGGGTTCCGCGCACATTTCCCCGAA
AAGTGCCACCTGACGTC

TABLE 34
Nucleotide Sequence of pcDNA ™ 6.2/cGeneBLAzer ™
GW/D-TOPO (See FIG. 34) (SEQ ID NO 156)

GACGGATCGGGAGATCTCCCGATCCCCTATGGTGCACTCTCAGTACAATC
TGCTCTGATGCCGCATAGTTAAGCCAGTATCTGCTCCCTGCTTGTGTGTT
GGAGGTCGCTGAGTAGTGCGCGAGCAAAATTTAAGCTACAACAAGGCAAG
GCTTGACCGACAATTGCATGAAGAATCTGCTTAGGGTTAGGCGTTTTGCG
CTGCTTCGCGATGTACGGGCCAGATATACGCGTTGACATTGATTATTGAC
TAGTTATTAATAGTAATCAATTACGGGGTCATTAGTTCATAGCCCATATA
TGGAGTTCCGCGTTACATAACTTACGGTAAATGGCCCGCCTGGCTGACCG
CCCAACGACCCCCGCCCATTGACGTCAATAATGACGTATGTTCCCATAGT
AACGCCAATAGGGACTTTCCATTGACGTCAATGGGTGGAGTATTTACGGT
AAACTGCCCACTTGGCAGTACATCAAGTGTATCATATGCCAAGTACGCCC
CCTATTGACGTCAATGACGGTAAATGGCCCGCCTGGCATTATGCCCAGTA
CATGACCTTATGGGACTTTCCTACTTGGCAGTACATCTACGTATTAGTCA
TCGCTATTACCATGGTGATGCGGTTTTGGCAGTACATCAATGGGCGTGGA
TAGCGGTTTGACTCACGGGGATTTCCAAGTCTCCACCCCATTGACGTCAA
TGGGAGTTTGTTTTGGCACCAAAATCAACGGGACTTTCCAAAATGTCGTA
ACAACTCCGCCCCATTGACGCAAATGGGCGGTAGGCGTGTACGGTGGGAG
GTCTATATAAGCAGAGCTCTCTGGCTAACTAGAGAACCCACTGCTTACTG
GCTTATCGAAATTAATACGACTCACTATAGGGAGACCCAAGCTGGCTAGT
TAAGCTGAGCATCAACAAGTTTGTACAAAAAAGCAGGCTCCGCGGCCGCC
CCCTTCACCAAGGGTGGGCGCGCCGACCCAGCTTTCTTGTACAAAGTGGT
TGATGCTGTTATGGACCCAGAAACGCTGGTGAAAGTAAAAGATGCTGAAG
ATCAGTTGGGTGCACGAGTGGGTTACATCGAACTGGATCTCAACAGCGGT
AAGATCCTTGAGAGTTTTCGCCCCGAAGAACGTTTTCCAATGATGAGCAC
TTTTAAAGTTCTGCTATGTGGCGCGGTATTATCCCGTATTGACGCCGGGC
AAGAGCAACTCGGTCGCCGCATACACTATTCTCAGAATGACTTGGTTGAG
TACTCACCAGTCACAGAAAAGCATCTTACGGATGGCATGACAGTAAGAGA
ATTATGCAGTGCTGCCATAACCATGAGTGATAACACTGCGGCCAACTTAC
TTCTGACAACGATCGGAGGACCGAAGGAGCTAACCGCTTTTTTGCACAAC
ATGGGGGATCATGTAACTCGCCTTGATCGTTGGGAACCGGAGCTGAATGA
AGCCATACCAAACGACGAGCGTGAGACCACGATGCGTGTAGCAATGGCAA
CAACGTTGCGCAAACTATTAACTGGCGAACTACTTACTCTAGCTTCCCGG
CAACAATTAATAGACTGGATGGAGGCGGATAAAGTTGCAGGACCACTTCT
GCGCTCGGCCCTTCCGGCTGGCTGGTTTATTGCTGATAAATCTGGAGCCG
GTGAGCGTGGGTCTCGCGGTATCATTGCAGCACTGGGGCCAGATGGTAAG
CCCTCCCGTATCGTAGTTATCTACACGACGGGGAGTCAGGCAACTATGGA
TGAACGAAATAGACAGATCGCTGAGATAGGTGCGTCACTGATTAAGCATT
GGTAACCGGTTAGTAATGAGTTTAAACGGGGGAGGCTAACTGAAACACGG
AAGGAGACAATACCGGAAGGAACCCGCGCTATGACGGCAATAAAAAGACA
GAATAAAACGCACGGGTGTTGGGTCGTTTGTTCATAAACGCGGGGTTCGG
TCCCAGGGCTGGCACTCTGTCGATACCCCACCGAGACCCCATTGGGGGCA
ATACGCCCGCGTTTCTTCCTTTTCCCCACCCCACCCCCCAAGTTCGGGTG
AAGGCCCAGGGCTCGCAGCCAACGTCGGGGCGGCAGGCCCTGCCATAGCA
GATCTGCGCAGCTGGGGCTCTAGGGGGTATCCCCACGCGCCCTGTAGCGG
CGCATTAAGCGCGGCGGGTGTGGTGGTTACGCGCAGCGTGACCGCTACAC
TTGCCAGCGCCCTAGCGCCCGCTCCTTTCGCTTTCTTCCCTTCCTTTCTC
GCCACGTTCGCCGGCTTTCCCCGTCAAGCTCTAAATCGGGGCATCCCTTT
AGGGTTCCGATTTAGTGCTTTACGGCACCTCGACCCCAAAAAACTTGATT
AGGGTGATGGTTCACGTAGTGGGCCATCGCCCTGATAGACGGTTTTTCGC
CCTTTGACGTTGGAGTCCACGTTCTTTAATAGTGGACTCTTGTTCCAAAC
TGGAACAACACTCAACCCTATCTCGGTCTATTCTTTTGATTTATAAGGGA
TTTTGGGGATTTCGGCCTATTGGTTAAAAAATGAGCTGATTTAACAAAAA
TTTAACGCGAATTAATTCTGTGGAATGTGTGTCAGTTAGGGTGTGGAAAG
TCCCCAGGCTCCCCAGCAGGCAGAAGTATGCAAAGCATGCATCTCAATTA
GTCAGCAACCAGGTGTGGAAAGTCCCCAGGCTCCCCAGCAGGCAGAAGTA
TGCAAAGCATGCATCTCAATTAGTCAGCAACCATAGTCCCGCCCCTAACT
CCGCCCATCCCGCCCCTAACTCCGCCCAGTTCCGCCCATTCTCCGCCCCA
TGGCTGACTAATTTTTTTTATTTATGCAGAGGCCGAGGCCGCCTCTGCCT
CTGAGCTATTCCAGAAGTAGTGAGGAGGCTTTTTTGGAGGCCTAGGCTTT
TGCAAAAAGCTCCCGGGAGCTTGTATATCCATTTTCGGATCTGATCAGCA
CGTGTTGACAATTAATCATCGGCATAGTATATCGGCATAGTATAATACGA
CAAGGTGAGGAACTAAACCATGGCCAAGCCTTTGTCTCAAGAAGAATCCA
CCCTCATTGAAAGAGCAACGGCTACAATCAACAGCATCCCCATCTCTGAA
GACTACAGCGTCGCCAGCGCAGCTCTCTCTAGCGACGGCCGCATCTTCAC
TGGTGTCAATGTATATCATTTTACTGGGGGACCTTGTGCAGAACTCGTGG
TGCTGGGCACTGGTGCTGCTGCGGCAGCTGGCAACCTGACTTGTATCGTC
GCGATCGGAAATGAGAACAGGGGCATCTTGAGCCCCTGCGGACGGTGCCG
ACAGGTGCTTCTCGATCTGCATCCTGGGATCAAAGGCATAGTGAAGGACA
GTGATGGACAGCCGACGGCAGTTGGGATTCGTGAATTGCTGCCCTCTGGT
TATGTGTGGGAGGGCTAAGCACTTCGTGGCCGAGGAGCAGGACTGACACG
TGCTACGAGATTTCGATTCCACGGCCGCGTTCTATGAAAGGTTGGGCTTC
GGAATCGTTTTCCGGGACGCCGGCTGGATGATCCTCCAGCGCGGGGATCT
CATGCTGGAGTTCTTCGCCCACCCCAACTTGTTTATTGCAGCTTATAATG
GTTACAAATAAAGCAATAGGATCACAAATTTCACAAATAAAGCATTTTTT
TCACTGCATTcTAGTTGTGGTTTGTCCAAACTCATCAATGTATGTTATCA
TGTCTGTATACCGTCGACCTCTAGCTAGAGCTTGGCGTAATCATGGTCAT
AGCTGTTTCCTGTGTGAAATTGTTATCGGCTCACAATTCCACACAACATA
GGAGCCGGAAGCATAAAGTGTAAAGCCTGGGGTGCCTAATGAGTGAGCTA
ACTCACATTAATTGCGTTGCGCTCACTGGCCGCTTTCCAGTCGGGAAACC
TGTCGTGCCAGCTGCATTAATGAATCGGCCAAGGCGCGGGGAGAGGCGGT
TTGCGTATTGGGCGCTCTTCCGCTTCCTCGCTCACTGACTCGCTGCGCTG
GGTCGTTCGGCTGCGGCGAGCGGTATCAGCTCACTCAAAGGCGGTAATAC
GGTTATCCACAGAATCAGGGGATAACGCAGGAAAGAAGATGTGAGCAAAA
GGCCAGCAAAAGGCCAGGAACCGTAAAAAGGCCGCGTTGCTGGCGTTTTT
CCATAGGCTCCGCCCCCCTGACGAGCATCACAAAAATCGACGCTCAAGTC
AGAGGTGGCGAAACCCGACAGGACTATAAAGATACCAGGCGTTTCCCCCT
GGAAGCTCCCTCGTGCGCTCTCCTGTTCCGACCCTGCCGCTTACCGGATA
CCTGTCCGCCTTTCTCCCTTCGGGAAGCGTGGCGCTTTCTCATAGCTCAC
GCTGTAGGTATCTCAGTTCGGTGTAGGTCGTTCGCTCCAAGCTGGGCTGT
GTGCACGAACGGCCGGTTCAGCCCGACCGCTGCGCCTTATCCGGTAACTA
TGGTCTTGAGTCCAACCCGGTAAGACACGAGTTATCGCCACTGGCAGCAG
CCACTGGTAACAGGATTAGCAGAGCGAGGTATGTAGGCGGTGCTACAGAG
TTCTTGAAGTGGTGGCCTAACTACGGCTACACTAGAAGAACAGTATTTGG
TATCTGCGCTCTGCTGAAGCCAGTTAGCTTCGGAAAAAGAGTTGGTAGCT
CTTGATCCGGCAAACAAACCACCGCTGGTAGCGGTTTTTTTGTTTGCAAG
CAGCAGATTACGCGCAGAAAAAAAGGATCTCAAGAAGATCCTTTGATCTT
TTCTACGGGGTCTGACGCTCAGTGGAACGAAAACTCACGTTAAGGGATTT
TGGTCATGAGATTATCAAAAAGGATCTTCACCTAGATCCTTTTAAATTAA
AAATGAAGTTTTAAATCAATCTAAAGTATATATGAGTAAACTTGGTCTGA
CAGTTACCAATGCTTAATCAGTGAGGCACGTATCTCAGCGATCTGTCTAT
TTCGTTCATCCATAGTTGCCTGACTCCCCGTCGTGTAGATAACTACGATA
CGGGAGGGCTTACCATCTGGCCCCAGTGCTGCAATGATACCGCGAGACCC
ACGCTCACCGGCTCCAGATTTATCAGCAATAAACCAGCCAGCCGGAAGGG
CCGAGCGCAGAAGTGGTCCTGCAACTTTATCCGCCTCCATCCAGTCTATT
AATTGTTGCCGGGAAGCTAGAGTAAGTAGTTCGCCAGTTAATAGTTTGCG
CAACGTTGTTGCCATTGCTAGAGGCATCGTGGTGTCACGCTCGTCGTTTG
GTATGGCTTCATTCAGCTCCGGTTCCGAACGATCAAGGGGAGTTACATGA
TCCCCCATGTTGTGCAAAAAAGCGGTTAGCTCCTTCGGTCCTCCGATCGT
TGTCAGAAGTAAGTTGGCCGCAGTGTTATCACTCATGGTTATGGCAGCAC
TGGATAATTCTCTTACTGTCATGCCATCCGTAAGATGCTTTTCTGTGACT
GGTGAGTACTCAACCAAGTCATTCTGAGAATAGTGTATGCGGCGACCGAG
TTGCTCTTGCCCGGCGTCAATACGGGATAATACCGCGCCACATAGCAGAA
CTTTAAAAGTGCTCATCATTGGAAAACGTTCTTCGGGGCGAAAACTCTCA
AGGATCTTACCGCTGTTGAGATCCAGTTCGATGTAACCCACTCGTGCACC
CAACTGATCTTCAGCATCTTTTACTTTCACCAGCGTTTCTGGGTGAGCAA
AAACAGGAAGGCAAAATGCCGCAAAAAAGGGAATAAGGGCGACACGGAAA
TGTTGAATACTCATACTCTTCCTTTTTCAATATTATTGAAGCATTTATCA
GGGTTATTGTCTCATGAGCGGATACATATTTGAATGTATTTAGAAAAATA
AACAAATAGGGGTTCCGCGCACATTTCCCCGAAAAGTGCGACCTGAGGTC

TABLE 35
Nucleotide Sequence of pcDNA ™ 6.2/nGeneBLAzer ™
GW/D-TOPO (See FIG. 35) (SEQ ID NO 157)

GACGGATCGGGAGATCTCCCGATCCCCTATGGTGCACTCTCAGTACAATC
TGCTCTGATGCCGCATAGTTAAGCCAGTATCTGCTCCCTGCTTGTGTGTT
GGAGGTCGCTGAGTAGTGCGCGAGCAAAATTTAAGCTACAACAAGGCAAG
GCTTGACCGACAATTGGATGAAGAATGTGCTTAGGGTTAGGGGTTTTGCG
CTGCTTCGCGATGTACGGGCCAGATATACGCGTTGACATTGATTATTGAC
TAGTTATTAATAGTAATCAATTACGGGGTCATTAGTTCATAGCCCATATA
TGGAGTTCCGCGTTACATAACTTACGGTAAATGGCCCGCCTGGCTGACCG
CCGAACGACCCCCGCCCATTGACGTCAATAATGACGTATGTTCCCATAGT
AACGCCAATAGGGACTTTCCATTGACGTCAATGGGTGGAGTATTTACGGT
AAACTGCCCACTTGGCAGTACATCAAGTGTATCATATGCCAAGTACGCCC
CCTATTGACGTCAATGACGGTAAATGGCCCGCCTGGCATTATGCCCAGTA
CATGACCTTATGGGACTTTCCTACTTGGCAGTACATCTACGTATTAGTCA
TCGCTATTACCATGGTGATGCGGTTTTGGCAGTACATCAATGGGCGTGGA
TAGCGGTTTGACTCACGGGGATTTCCAAGTCTCCACCCCATTGACGTCAA
TGGGAGTTTGTTTTGGCACCAAAATCAACGGGACTTTCCAAAATGTCGTA
ACAACTCCGCCCCATTGACGCAAATGGGCGGTAGGCGTGTACGGTGGGAG
GTCTATATAAGCAGAGCTCTCTGGCTAACTAGAGAACCCACTGCTTACTG
GCTTATCGAAATTAATACGAGTCACTATAGGGAGACCCAAGCTGGCTAGT
TAAGCTGAGCATCAACAAGTTTGTACAAAAAAGCAGGCTCCGCGGCCGCC
CCCTTCACCAAGGGTGGGCGCGCCGACCCAGCTTTCTTGTACAAAGTGGT
TGATGCTGTTATGGACCCAGAAACGCTGGTGAAAGTAAAAGATGCTGAAG
ATCAGTTGGGTGCACGAGTGGGTTACATCGAACTGGATCTCAACAGCGGT
AAGATCCTTGAGAGTTTTCGCCCCGAAGAACGTTTTCCAATGATGAGCAC
TTTTAAAGTTCTGCTATGTGGCGCGGTATTATCCCGTATTGACGCCGGGC
AAGAGCAACTCGGTCGCCGCATACACTATTCTCAGAATGACTTGGTTGAG
TACTCACCAGTCACAGAAAAGCATCTTACGGATGGCATGACAGTAAGAGA
ATTATGCAGTGCTGCCATAACCATGAGTGATAACACTGCGGCCAACTTAC
TTCTGACAACGATCGGAGGACCGAAGGAGCTAACCGCTTTTTTGCACAAC
ATGGGGGATCATGTAACTCGCCTTGATCGTTGGGAACCGGAGCTGAATGA
AGCCATACCAAACGACGAGCGTGACACCACGATGCCTGTAGCAATGGCAA
CAACGTTGCGCAAACTATTAACTGGCGAACTACTTACTCTAGCTTCCCGG
CAACAATTAATAGACTGGATGGAGGCGGATAAAGTTGCAGGACCACTTCT
GCGCTCGGCCCTTCCGGCTGGCTGGTTTATTGCTGATAAATCTGGAGCCG
GTGAGCGTGGGTCTCGCGGTATCATTGCAGCACTGGGGCCAGATGGTAAG
CCCTCCCGTATCGTAGTTATCTACACGACGGGGAGTCAGGCAACTATGGA
TGAACGAAATAGACAGATCGCTGAGATAGGTGCCTCACTGATTAAGCATT
GGTAACCGGTTAGTAATGAGTTTAAACGGGGGAGGCTAACTGAAACACGG
AAGGAGACAATACCGGAAGGAACCCGCGCTATGACGGCAATAAAAAGACA
GAATAAAACGCACGGGTGTTGGGTCGTTTGTTCATAAACGCGGGGTTCGG
TCCCAGGGCTGGCACTCTGTCGATACCCCACCGAGACCCCATTGGGGGCA
ATACGCCCGCGTTTCTTCCTTTTCCCCACCCCACCCCCCAAGTTCGGGTG
AAGGCCCAGGGCTCGCAGCCAACGTCGGGGCGGCAGGCCCTGCCATAGCA
GATCTGGGCAGCTGGGGCTCTAGGGGGTATCCCCACGCGCCCTGTAGCGG
CGCATTAAGCGCGGCGGGTGTGGTGGTTACGCGCAGCGTGACCGCTACAC
TTGCCAGCGCCCTAGCGCGCGCTCCTTTCGCTTTCTTCCCTTCCTTTCTC
GCCACGTTCGCCGGCTTTCCCCGTCAAGCTCTAAATCGGGGCATCCCTTT
AGGGTTCCGATTTAGTGCTTTACGGCACCTCGAGCCCAAAAAAGTTGATT
AGGGTGATGGTTCACGTAGTGGGCCATCGCCCTGATAGACGGTTTTTCGC
CCTTTGACGTTGGAGTCCACGTTCTTTAATAGTGGACTCTTGTTCCAAAC
TGGAACAACACTCAACCCTATCTCGGTCTATTGTTTTGATTTATAAGGGA
TTTTGGGGATTTCGGCCTATTGGTTAAAAAATGAGCTGATTTAACAAAAA
TTTAACGCGAATTAATTCTGTGGAATGTGTGTCAGTTAGGGTGTGGAAAG
TCCCCAGGCTCCCCAGCAGGCAGAAGTATGCAAAGCATGCATCTCAATTA
GTCAGCAACCAGGTGTGGAAAGTCCGCAGGCTCCCCAGCAGGCAGAAGTA
TGCAAAGCATGGATCTCAATTAGTCAGCAACCATAGTCCCGCCCCTAACT
CCGCCCATCCCGCCCCTAACTCCGGCCAGTTCCGCCCATTCTCCGCCGCA
TGGGTGAGTAATTTTTTTTATTTATGCAGAGGCCGAGGCCGCCTCTGCCT
CTGAGCTATTCCAGAAGTAGTGAGGAGGCTTTTTTGGAGGCCTAGGCTTT
TGGAAAAAGCTCCCGGGAGCTTGTATATCCATTTTCGGATCTGATCAGCA
CGTGTTGACAATTAATCATCGGCATAGTATATCGGCATAGTATAATACGA
CAAGGTGAGGAACTAAACCATGGCCAAGCCTTTGTCTCAAGAAGAATCCA
CCCTCATTGAAAGAGCAACGGCTACAATCAACAGCATCCCCATCTCTGAA
GACTACAGCGTCGCCAGCGCAGCTCTCTCTAGCGACGGCCGCATCTTCAC
TGGTGTCAATGTATATCATTTTACTGGGGGACCTTGTGCAGAACTCGTGG
TGCTGGGCACTGCTGCTGCTGCGGCAGCTGGCAACCTGACTTGTATCGTC
GCGATCGGAAATGAGAACAGGGGCATCTTGAGCCCCTGCGGACGGTGCCG
ACAGGTGCTTCTGGATCTGCATCGTGGGATCAAAGCCATAGTGAAGGACA
GTGATGGACAGCCGACGGCAGTTGGGATTGGTGAATTGCTGCCCTCTGGT
TATGTGTGGGAGGGCTAAGCACTTCGTGGCCGAGGAGCAGGACTGACACG
TGCTACGAGATTTCGATTCCACCGCCGCCTTCTATGAAAGGTTGGGCTTC
GGAATCGTTTTCCGGGACGCCGGCTGGATGATCGTGCAGCGCGGGGATCT
CATGCTGGAGTTCTTCGCCCACCCCAACTTGTTTATTGCAGCTTATAATG
GTTACAAATAAAGCAATAGCATCACAAATTTCACAAATAAAGCATTTTTT
TCACTGCATTCTAGTTGTGGTTTGTCCAAACTCATCAATGTATCTTATCA
TGTCTGTATACCGTCGACCTCTAGCTAGAGCTTGGCGTAATCATGGTCAT
AGCTGTTTCCTGTGTGAAATTGTTATCCGCTCACAATTCCACACAACATA
CGAGCCGGAAGCATAAAGTGTAAAGCCTGGGGTGCCTAATGAGTGAGCTA
ACTCACATTAATTGCGTTGCGCTCACTGCCCGCTTTCCAGTCGGGAAACC
TGTCGTGCCAGCTGCATTAATGAATCGGCCAACGCGCGGGGAGAGGCGGT
TTGCGTATTGGGCGCTCTTCCGCTTCCTCGCTCACTGACTCGCTGCGCTC
GGTCGTTCGGGTGCGGCGAGCGGTATCAGCTCACTCAAAGGCGGTAATAC
GGTTATCCACAGAATCAGGGGATAACGCAGGANAGAACATGTGAGCAAAA
GGCCAGCAAAAGGCCAGGAACCGTAAAAAGGCCGCGTTGCTGGCGTTTTT
CCATAGGCTCCGCCCCCCTGACGAGCATCACAAAAATCGACGCTCAAGTC
AGAGGTGGCGAAACCCGACAGGACTATAAAGATACCAGGCGTTTCCCCCT
GGAAGCTCCCTCGTGCGCTCTCCTGTTCCGACCCTGCCGCTTACCGGATA
CCTGTCCGCCTTTCTCCCTTCGGGAAGCGTGGCGCTTTCTCATAGCTCAC
GCTGTAGGTATCTCAGTTCGGTGTAGGTCGTTCGCTCCAAGCTGGGCTGT
GTGCACGAACCCCCCGTTCAGCCCGACCGCTGCGCCTTATCCGGTAACTA
TCGTCTTGAGTCCAACCCGGTAAGACACGACTTATCGCCACTGGCAGCAG
CCACTGGTAACAGGATTAGCAGAGCGAGGTATGTAGGCGGTGCTACAGAG
TTCTTGAAGTGGTGGCCTAACTACGGCTACACTAGAAGAACAGTATTTGG
TATCTGCGCTCTGCTGAAGCCAGTTACCTTCGGAAAAAGAGTTGGTAGCT
CTTGATCCGGCAAACAAACCACCGCTGGTAGCGGTTTTTTTGTTTGCAAG
CAGCAGATTACGCGCAGAAAAAAAGGATCTCAAGAAGATCCTTTGATCTT
TTCTACGGGGTCTGACGCTCAGTGGAACGAAAACTCACGTTAAGGGATTT
TGGTCATGAGATTATCAAAAAGGATCTTCACCTAGATCCTTTTAAATTAA
AAATGAAGTTTTAAATCAATCTAAAGTATATATGAGTAAACTTGGTCTGA
CAGTTACCAATGCTTAATCAGTGAGGCACCTATCTCAGCGATCTGTCTAT
TTCGTTCATCCATAGTTGCCTGACTCCCCGTCGTGTAGATAACTACGATA
CGGGAGGGCTTACCATCTGGCCCCAGTGCTGCAATGATACCGCGAGACCC
ACGCTCACCGGCTCCAGATTTATCAGCAATAAACCAGCCAGCCGGAAGGG
CCGAGCGCAGAAGTGGTCCTGCAACTTTATCCGCCTCCATCCAGTCTATT
AATTGTTGCCGGGAAGCTAGAGTAAGTAGTTCGCCAGTTAATAGTTTGCG
CAACGTTGTTGCCATTGCTACAGGCATCGTGGTGTCACGCTCGTCGTTTG
GTATGGCTTCATTCAGCTCCGGTTCCCAACGATCAAGGCGAGTTACATGA
TCCCCGATGTTGTGCAAAAAAGCGGTTAGCTCCTTCGGTCCTCCGATCGT
TGTCAGAAGTAAGTTGGCCGCAGTGTTATCACTCATGGTTATGGCAGCAC
TGCATAATTCTCTTACTGTCATGCCATCCGTAAGATGCTTTTCTGTGACT
GGTGAGTACTCAACCAAGTCATTCTGAGAATAGTGTATGCGGCGACCGAG
TTGCTCTTGCCCGGCGTCAATACGGGATAATACCGCGCCACATAGCAGAA
CTTTAAAAGTGCTCATCATTGGAAAACGTTCTTCGGGGCGAAAACTCTCA
AGGATCTTACCGCTGTTGAGATGGAGTTCGATGTAACGCACTCGTGCACC
CAACTGATCTTCAGCATCTTTTACTTTCACCAGCGTTTCTGGGTGAGCAA
AAACAGGAAGGCAAAATGCCGCAAAAAAGGGAATAAGGGCGACACGGAAA
TGTTGAATACTCATACTCTTCCTTTTTCAATATTATTGAAGCATTTATCA
GGGTTATTGTCTCATGAGCGGATACATATTTGAATGTATTTAGAAAAATA
AACAAATAGGGGTTCCGCGCACATTTCCCCGAAAAGTGCCACCTGACGTC

TABLE 36
Nucleotide Sequence of pENTR/GeneBLAzer ™
(See FIG. 41) (SEQ ID NO: 158)

CTTTCCTGCGTTATCCCCTGATTCTGTGGATAACCGTATTACCGCCTTTG
AGTGAGCTGATACCGCTCGCCGCAGCCGAACGACCGAGCGCAGCGAGTCA
GTGAGCGAGGAAGCGGAAGAGCGCCCAATACGCAAACCGCCTCTCCCCGC
GCGTTGGCCGATTCATTAATGCAGCTGGCACGACAGGTTTCCCGACTGGA
AAGCGGGCAGTGAGCGCAACGCAATTAATACGCGTACCGCTAGCCAGGAA
GAGTTTGTAGAAACGCAAAAAGGCCATCCGTCAGGATGGCCTTCTGCTTA
GTTTGATGCCTGGCAGTTTATGGCGGGCGTCCTGCCCGCCACCCTCCGGG
CCGTTGCTTCACAACGTTCAAATCCGCTCCCGGCGGATTTGTCCTACTCA
GGAGAGCGTTCACCGACAAACAACAGATAAAACGAAAGGCCCAGTCTTCC
GACTGAGCCTTTCGTTTTATTTGATGCCTGGCAGTTCCCTACTCTCGCGT
TAACGCTAGCATGGATGTTTTCCCAGTCACGACGTTGTAAAACGACGGCC
AGTCTTAAGCTCGGGCCCCAAATAATGATTTTATTTTGACTGATAGTGAC
CTGTTCGTTGCAACAAATTGATGAGCAATGCTTTTTTATAATGCCAACTT
TGTACAAAAAAGCAGGCACCATGGACCCAGAAAGGCTGGTGAAAGTAAAA
GATGCTGAAGATCAGTTGGGTGCACGAGTGGGTTACATCGAACTGGATCT
CAACAGCGGTAAGATCCTTGAGAGTTTTCGCCCCGAAGAACGTTTTCCAA
TGATGAGCACTTTTAAAGTTCTGGTATGTGGCGCGGTATTATCCCGTATT
GACGCCGGGCAAGAGCAACTCGGTCGCCGCATACACTATTCTCAGAATGA
CTTGGTTGAGTACTCACCAGTCACAGAAAAGCATCTTACGGATGGCATGA
CAGTAAGAGAATTATGCAGTGCTGCCATAACCATGAGTGATAACACTGCG
GCGAACTTACTTCTGACAACGATCGGAGGACCGAAGGAGCTAACCGCTTT
TTTGCACAACATGGGGGATCATGTAACTCGCCTTGATCGTTGGGAACCGG
AGCTGAATGAAGCCATACCAAACGACGAGCGTGACACCACGATGCCTGTA
GCAATGGCAACAACGTTGCGCAAACTATTAACTGGCGAACTACTTACTCT
AGCTTCCCGGCAACAATTAATAGACTGGATGGAGGCGGATAAAGTTGCAG
GACCACTTCTGCGCTCGGCCCTTCCGGCTGGCTGGTTTATTGCTGATAAA
TCTGGAGCCGGTGAGCGTGGGTCTCGCGGTATCATTGCAGCACTGGGGCC
AGATGGTAAGCCCTCCCGTATCGTAGTTATCTACACGACGGGGAGTCAGG
CAACTATGGATGAACGAAATAGACAGATCGCTGAGATAGGTGCCTCACTG
ATTAAGCATTGGTAGACAGCTTTCTTGTACAAAGTTGGCATTATAAGAAA
GCATTGCTTATCAATTTGTTGCAACGAACAGGTCACTATCAGTCAAAATA
AAATCATTATTTGCCATCCAGCTGATATCCGGTATAGTGAGTCGTATTAC
ATGGTCATAGCTGTTTCCTGGCAGCTCTGGCCCGTGTCTCAAAATCTCTG
ATGTTACATTGCACAAGATAAAAATATATCATCATGAAGAATAAAACTGT
CTGCTTACATAAACAGTAATACAAGGGGTGTTATGAGCCATATTCAACGG
GAAACGTCGAGGCCGCGATTAAATTCCAACATGGATGCTGATTTATATGG
GTATAAATGGGCTCGCGATAATGTCGGGCAATCAGGTGCGACAATCTATC
GCTTGTATGGGAAGCCCGATGCGCCAGAGTTGTTTCTGAAACATGGCAAA
GGTAGCGTTGCCAATGATGTTACAGATGAGATGGTCAGACTAAACTGGCT
GACGGAATTTATGCCTCTTCCGACCATCAAGCATTTTATCCGTACTCCTG
ATGATGCATGGTTACTCACCACTGCGATCCCCGGAAAAACAGCATTCCAG
GTATTAGAAGAATATCCTGATTCAGGTGAAAATATTGTTGATGCGCTGGC
AGTGTTCCTGCGCCGGTTGCATTCGATTCCTGTTTGTAATTGTCCTTTTA
ACAGCGATCGCGTATTTCGTCTCGCTCAGGCGCAATCACGAATGAATAAC
GGTTTGGTTGATGCGAGTGATTTTGATGACGAGCGTAATGGCTGGCCTGT
TGAACAAGTCTGGAAAGAAATGCATAAACTTTTGCCATTCTCACCGGATT
CAGTCGTCACTCATGGTGATTTCTCACTTGATAACCTTATTTTTGACGAG
GGGAAATTAATAGGTTGTATTGATGTTGGACGAGTCGGAATCGCAGACCG
ATACCAGGATCTTGCCATCCTATGGAACTGCCTCGGTGAGTTTTCTCCTT
CATTACAGAAACGGCTTTTTCAAAAATATGGTATTGATAATCCTGATATG
AATAAATTGCAGTTTCATTTGATGCTCGATGAGTTTTTCTAATCAGAATT
GGTTAATTGGTTGTAACACTGGCAGAGCATTACGCTGACTTGACGGGACG
GCGCAAGCTCATGACCAAAATCCCTTAACGTGAGTTACGCGTCGTTCCAC
TGAGCGTCAGACCCCGTAGAAAAGATCAAAGGATCTTCTTGAGATCCTTT
TTTTCTGCGCGTAATCTGCTGCTTGCAAACAAAAAAACCACCGCTACCAG
CGGTGGTTTGTTTGCCGGATCAAGAGCTACCAACTCTTTTTCCGAAGGTA
ACTGGCTTCAGCAGAGCGCAGATACCAAATACTGTCCTTCTAGTGTAGCC
GTAGTTAGGCCACCACTTCAAGAACTCTGTAGCACCGCCTACATACCTCG
CTCTGCTAATCCTGTTACCAGTGGCTGCTGCCAGTGGCGATAAGTCGTGT
CTTACCGGGTTGGACTCAAGACGATAGTTACCGGATAAGGCGCAGCGGTC
GGGCTGAACGGGGGGTTCGTGCACACAGCCCAGCTTGGAGCGAACGACCT
ACACCGAACTGAGATACCTACAGCGTGAGCATTGAGAAAGCGCCACGCTT
CCCGAAGGGAGAAAGGCGGACAGGTATCCGGTAAGCGGCAGGGTCGGAAC
AGGAGAGCGCACGAGGGAGCTTCCAGGGGGAAACGCCTGGTATCTTTATA
GTCCTGTCGGGTTTCGCCACCTCTGACTTGAGCGTCGATTTTTGTGATGC
TCGTCAGGGGGGCGGAGCCTATGGAAAAACGCCAGCAACGCGGCCTTTTT
ACGGTTCCTGGCCTTTTGCTGGCCTTTTGCTCACATGTT

TABLE 37

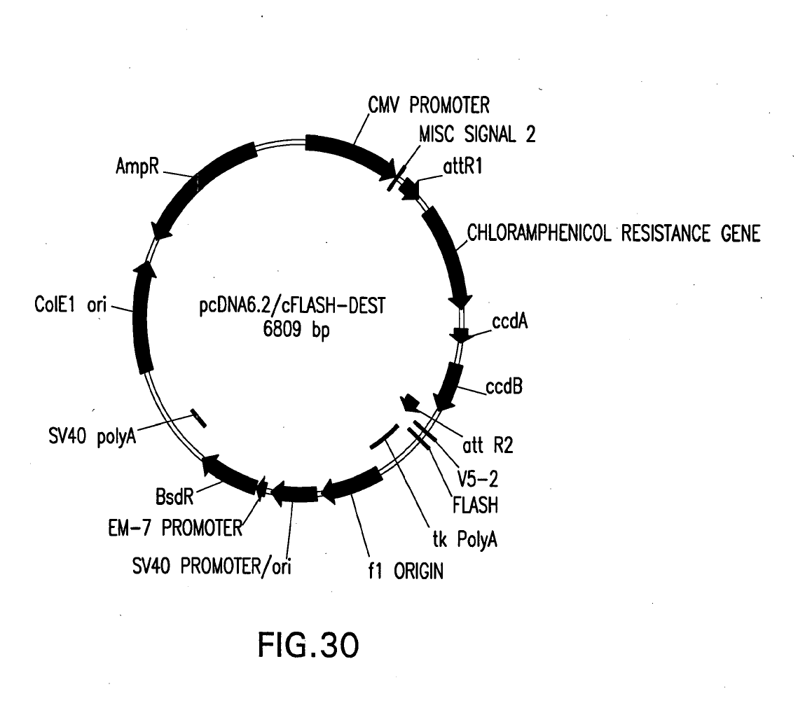

Nucleotide Sequence of pcDNA6.2/cFLASH-DEST
(See FIG. 30) (SEQ ID NO: 159)

| | |
|---|---|
| 1 | cgatgtacgg gccagatata cgcgttgaca ttgattattg actagttatt aatagtaatc |
| 61 | aattacgggg tcattagttc atagcccata tatggagttc cgcgttacat aacttacggt |
| 121 | aaatggcccg cctggctgac cgcccaacga cccccgccca ttgacgtcaa taatgacgta |
| 181 | tgttcccata gtaacgccaa tagggacttt ccattgacgt caatgggtgg agtatttacg |
| 241 | gtaaactgcc cacttggcag tacatcaagt gtatcatatg ccaagtacgc cccctattga |
| 301 | cgtcaatgac ggtaaatggc ccgcctggca ttatgcccag tacatgacct tatgggactt |
| 361 | tcctacttgg cagtacatct acgtattagt catcgctatt accatggtga tgcggttttg |
| 421 | gcagtacatc aatgggcgtg gatagcggtt tgactcacgg ggatttccaa gtctccaccc |
| 481 | cattgacgtc aatgggagtt tgttttggca ccaaaatcaa cgggactttc caaaatgtcg |
| 541 | taacaactcc gccccattga cgcaaatggg cggtaggcgt gtacggtggg aggtctatat |
| 601 | aagcagagct ctctggctaa ctagagaacc cactgcttac tggcttatcg aaattaatac |
| 661 | gactcactat agggagaccc aagctggcta gttaagctga gcatcaacaa gtttgtacaa |
| 721 | aaaagctgaa cgagaaacgt aaaatgatat aaatatcaat atattaaatt agattttgca |
| 781 | taaaaaacag actacataat actgtaaaac acaacatatc cagtcactat ggcggccgca |
| 841 | ttaggcaccc caggctftac actttatgct tccggctcgt ataatgtgtg gattttgagt |
| 901 | taggatccgg cgagattttc aggagctaag gaagctaaaa tggagaaaaa aatcactgga |
| 961 | tataccaccg ttgatatatc ccaatggcat cgtaaagaac attttgaggc atttcagtca |
| 1021 | gttgctcaat gtacctataa ccagaccgtt cagctggata ttacggcctt tttaaagacc |
| 1081 | gtaaagaaaa ataagcacaa gttttatccg gcctttattc acattcttgc ccgcctgatg |
| 1141 | aatgctcatc cggaattccg tatggcaatg aaagacggtg agctggtgat atgggatagt |
| 1201 | gttcaccctt gttacaccgt tttccatgag caaactgaaa cgttttcatc gctctggagt |
| 1261 | gaataccacg acgatttccg gcagtttcta cacatatatt cgcaagatgt ggcgtgttac |
| 1321 | ggtgaaaacc tggcctattt ccctaaaggg tttattgaga atatgttttt cgtctcagcc |
| 1381 | aatccctggg tgagtttcac cagttttgat ttaaacgtgg ccaatatgga caacttcttc |
| 1441 | gcccccgttt tcaccatggg caaatattat acgcaaggcg acaaggtgct gatgccgctg |
| 1501 | gcgattcagg ttcatcatgc cgtctgtgat ggcttccatg tcggcagaat gcttaatgaa |
| 1561 | ttacaacagt actgcgatga gtggcagggc ggggcgtaaa gatctggatc cggcttacta |
| 1621 | aaagccagat aacagtatgc gtatttgcgc gctgattttt gcggtataag aatatatact |
| 1681 | gatatgtata cccgaagtat gtcaaaaaga ggtgtgctat gaagcagcgt attacagtga |
| 1741 | cagttgacag cgacagctat cagttgctca aggcatatat gatgtcaata tctccggtct |
| 1801 | ggtaagcaca accatgcaga atgaagcccg tcgtctgcgt gccgaacgct ggaaagcgga |
| 1861 | aaatcaggaa gggatggctg aggtcgcccg gtttattgaa atgaacggct ctfftgctga |
| 1921 | cgagaacagg gactggtgaa atgcagttta aggtttacac ctataaaaga gagagccgtt |
| 1981 | atcgtctgtt tgtggatgta cagagtgata ttattgacac gcccgggcga cggatggtga |
| 2041 | tccccctggc cagtgcacgt ctgctgtcag ataaagtctc ccgtgaactt tacccggtgg |
| 2101 | tgcatatcgg ggatgaaagc tggcgcatga tgaccaccga tatggccagt gtgccggtct |
| 2161 | ccgttatcgg ggaagaagtg gctgatctca gccaccgcga aaatgacatc aaaaacgcca |
| 2221 | ttaacctgat gttctgggga atataaatgt caggctccgt tatacacagc cagtctgcag |
| 2281 | gtcgaccata gtgactggat atgttgtgtt ttacagtatt atgtagtctg ttttttatgc |
| 2341 | aaaatctaat ttaatatatt gataiftata tcattttacg tttctcgttc agctttcttg |
| 2401 | tacaaagtgg ttgatgctgt taacgggaag cctatcccta accctctcct cggtctcgat |
| 2461 | tetacgcgta ccggtgctgg tggctgttgt ectggctgtt gcggtggegg ctagtaatga |
| 2521 | gtttaaaegg gggaggctaa ctgaaacacg gaaggagaca ataccggaag gaaeccgcgc |
| 2581 | tatgaeggca ataaaaagac agaataaaae gcacgggtgt tgggtcgttt gttcataaac |
| 2641 | gcggggttcg gtcccaggge tggcaetetg tegataccee accgagaccc cattggggcc |
| 2701 | aataegeecg egtttcttec ttttccccac eccaccecec aagttcgggt gaaggcccag |
| 2761 | ggctegcage caaegteggg geggcaggcc etgeeatagc agatetgcge agetggggct |
| 2821 | ctagggggta tecccacgcg ccctgtageg gcgcattaag cgcggcgggt gtggtggtta |
| 2881 | cgcgcagcgt gacegetaca ettgecagcg ecctagcgee cgctectttc gctttcttce |
| 2941 | cttcctttct cgccaegttc gccggetttc cccgtcaagc tctaaatcgg ggcatcectt |
| 3001 | tagggttceg atttagtgct ttacggcacc tcgaecceaa aaaaettgat tagggtgatg |
| 3061 | gttcaegtag tgggeeateg ecetgataga eggttttteg eectttgaeg ttggagtcca |
| 3121 | cgttctttaa tagtggactc ttgttccaaa ctggaacaac actcaaccct atctcggtct |
| 3181 | attettttga tttataaggg attttgggga ttteggccta ttggttaaaa aatgagctga |
| 3241 | tttaacaaaa atttaacgcg aattaattct gtggaatgtg tgtcagttag ggtgtggaaa |
| 3301 | gtccceaggc teeccageag geagaagtat geaaageatg cateteaatt agtcagcaac |
| 3361 | eaggtgtgga aagtceeeag getececage aggcagaagt atgcaaagea tgcatcteaa |
| 3421 | ttagtcagca aecatagtcc cggccctaae tcgggcgate ccgcccctaa ctccgcccag |
| 3481 | ttccgcccat tcteegcccc atggctgact aatttttttt atttatgcag aggeegagge |
| 3541 | cgcetetgcc tetgagetat teeagaagta gtgaggaggc ttttttggag gectaggctt |
| 3601 | ttgcaaaaag ctcccgggag ettgtatatc cattttegga tetgateage acgtgttgac |
| 3661 | aattaateat eggeatagta tateggeata gtataataeg aeaaggtgag gaactaaacc |
| 3721 | atggeeaagc etttgtctca agaagaatec acectcattg aaagagcaac ggctacaatc |
| 3781 | aacagcatcc ccatetctga agactacagc gtcgccagcg cagctctctc tagcgacggc |
| 3841 | cgeatettca etggtgteaa tgtatatcat tttaetgggg gaeettgtge agaactcgtg |
| 3901 | gtgetgggea etgetgetge tgeggeagct ggeaaeetga ettgtatcgt egegategga |
| 3961 | aatgagaaca ggggeatctt gagcccctge ggaeggtgee gaeaggtgct tctcgatctg |
| 4021 | catcctggga tcaaagccat agtgaaggac agtgatggac agccgacggc agttgggatt |
| 4081 | cgtgaattgc tgccctctgg ttatgtgtgg gagggctaag cacttcgtgg ccgaggagca |
| 4141 | ggactgacac gtgctacgag atttcgattc caccgccgcc ttctatgaaa ggttgggctt |
| 4201 | cggaatcgtt ttccgggacg ccggctggat gatcctccag cgcggggatc tcatgctgga |
| 4261 | gttcttcgcc caccccaact tgtttattgc agcttataat ggttacaaat aaagcaatag |
| 4321 | catcacaaat ttcacaaata aagcattttt ttcactgcat tctagttgtg gtttgtccaa |
| 4381 | actcatcaat gtatcttatc atgtctgtat accgtcgacc tctagctaga gcttggcgta |
| 4441 | atcatggtca tagctgtttc ctgtgtgaaa ttgttatccg ctcacaattc cacacaacat |
| 4501 | acgagccgga agcataaagt gtaaagcctg gggtgcctaa tgagtgagct aactcacatt |
| 4561 | aattgcgttg cgctcactgc ccgctttcca gtcgggaaac ctgtcgtgcc agctgcatta |
| 4621 | atgaatcggc caacgcgcgg ggagaggcgg tttgcgtatt gggcgctctt ccgcttcctc |
| 4681 | gctcactgac tcgctgcgct cggtcgttcg gctgcggcga gcggtatcag otcactcaaa |
| 4741 | ggcggtaata cggttatcca cagaatcagg ggataacgca ggaaagaaca tgtgagcaaa |
| 4801 | aggccagcaa aaggccagga accgtaaaaa ggccgcgttg ctggcgtttt tccataggct |
| 4861 | ccgcccccct gacgagcatc acaaaaatcg acgctcaagt cagaggtggc gaaacccgac |
| 4921 | aggactataa agataccagg cgtttccccc tggaagctcc ctcgtgcgct ctcctgttcc |
| 4981 | gaccctgccg cttaccggat acctgtccgc ctttctccct tcgggaagcg tggcgctttc |
| 5041 | tcatagctca cgctgtaggt atctcagttc ggtgtaggtc gttcgctcca agctgggctg |
| 5101 | tgtgcacgaa ccccccgttc agcccgaccg ctgcgcctta tccggtaact atcgtcttga |
| 5161 | gtccaacccg gtaagacacg acttatcgcc actggcagca gccactggta acaggattag |
| 5221 | cagagcgagg tatgtaggcg gtgctacaga gttcttgaag tggtggccta actacggcta |
| 5281 | cactagaaga acagtatttg gtatctgcgc tctgctgaag ccagttacct tcggaaaaag |
| 5341 | agttggtagc tcttgatccg gcaaacaaac caccgctggt agcggttttt ttgtttgcaa |
| 5401 | gcagcagatt acgcgcagaa aaaaaggatc tcaagaagat cctttgatct tttctacggg |
| 5461 | gtctgacgct cagtggaacg aaaactcacg ttaagggatt ttggtcatga gattatcaaa |
| 5521 | aaggatcttc acctagatcc ttttaaatta aaaatgaagt tttaaatcaa tctaaagtat |
| 5581 | atatgagtaa acttggtctg acagttacca atgcttaatc agtgaggcac ctatctcagc |
| 5641 | gatctgtcta tttcgttcat ccatagttgc ctgactcccc gtcgtgtaga taactacgat |
| 5701 | acgggagggc ttaccatctg gccccagtgc tgcaatgata ccgcgagacc cacgctcacc |
| 5761 | ggctccagat ttatcagcaa taaaccagcc agccggaagg gccgagcgca gaagtggtcc |
| 5821 | tgcaacttta tccgcctcca tccagtctat taattgttgc cgggaagcta gagtaagtag |
| 5881 | ttcgccagtt aatagtttgc gcaacgttgt tgccattgct acaggcatcg tggtgtcacg |
| 5941 | ctcgtcgttt ggtatggctt cattcagctc cggttcccaa cgatcaaggc gagttacatg |
| 6001 | atcccccatg ttgtgcaaaa aagcggttag ctccttcggt cctccgatcg ttgtcagaag |
| 6061 | taagttggcc gcagtgttat cactcatggt tatggcagca ctgcataatt ctcttactgt |
| 6121 | catgccatcc gtaagatgct tttctgtgac tggtgagtac tcaaccaagt cattctgaga |
| 6181 | atagtgtatg cggcgaccga gttgctcttg cccggcgtca atacgggata ataccgcgcc |
| 6241 | acatagcaga actttaaaag tgctcatcat tggaaaacgt tcttcggggc gaaaactctc |
| 6301 | aaggatctta ccgctgttga gatccagttc gatgtaaccc actcgtgcac ccaactgatc |
| 6361 | ttcagcatct tttactttca ccagcgtttc tgggtgagca aaaacaggaa ggcaaaatgc |
| 6421 | cgcaaaaaag ggaataaggg cgacacggaa atgttgaata ctcatactct tcctttttca |
| 6481 | atattattga agcatttatc agggttattg tctcatgagc ggatacatat ttgaatgtat |
| 6541 | ttagaaaaat aaacaaatag gggttccgcg cacatttccc cgaaaagtgc cacctgacgt |
| 6601 | cgacggatcg ggagatctcc cgatccccta tggtgcactc tcagtacaat ctgctctgat |
| 6661 | gccgcatagt taagccagta tctgctccct gcttgtgtgt tggaggtcgc tgagtagtgc |
| 6721 | gcgagcaaaa tttaagctac aacaaggcaa ggcttgaccg acaattgcat gaagaatctg |
| 6781 | cttagggtta ggcgttttgc gctgcttcg |

TABLE 38

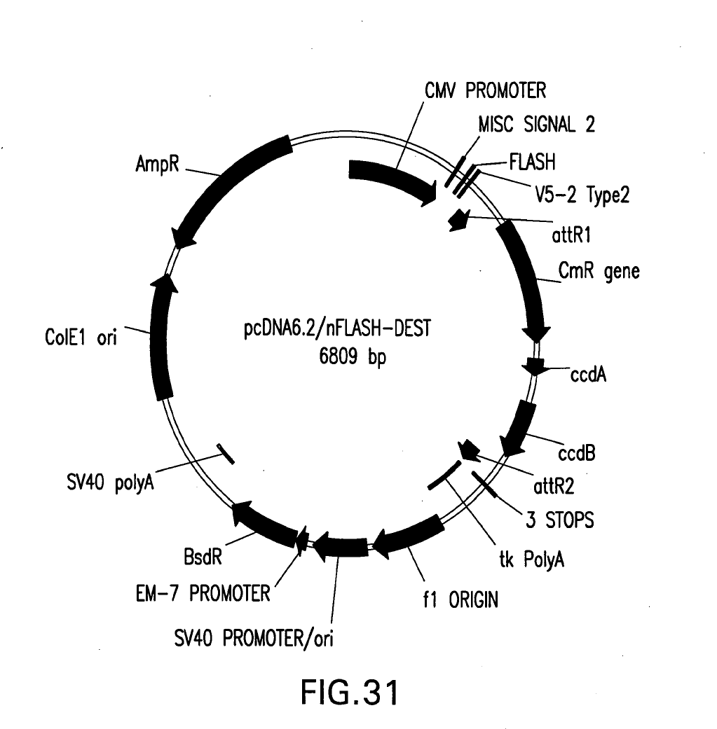

Nucleotide Sequence of pcDNA6.2/nELASH-DEST
(See FIG. 31) (SEQ ID NO: 160)

| | |
|---|---|
| 1 | cgatgtacgg gccagatata cgcgttgaca ttgattattg actagttatt aatagtaatc |
| 61 | aattacgggg tcattagttc atagcccata tatggagttc cgcgttacat aacttacggt |
| 121 | aaatggcccg cctggctgac cgcccaacga cccccgccca ttgacgtcaa taatgacgta |
| 181 | tgttcccata gtaacgccaa tagggacttt ccattgacgt caatgggtgg agtatttacg |
| 241 | gtaaactgcc cacttggcag tacatcaagt gtatcatatg ccaagtacgc cccctattga |
| 301 | cgtcaatgac ggtaaatggc ccgcctggca ttatgcccag tacatgacct tatgggactt |
| 361 | tcctacttgg cagtacatct acgtattagt catcgctatt accatggtga tgcggttttg |
| 421 | gcagtacatc aatgggcgtg gatagcggtt tgactcacgg ggatttccaa gtctccaccc |
| 481 | cattgacgtc aatgggagtt tgttttggca ccaaaatcaa cgggactttc caaaatgtcg |
| 541 | taacaactcc gccccattga cgcaaatggg cggtaggcgt gtacggtggg aggtctatat |
| 601 | aagcagagct ctctggctaa ctagagaacc cactgcttac tggcttatcg aaattaatac |
| 661 | gactcactat agggagaccc aagctggcta gttaagctgc accatggctg gtggctgttg |
| 721 | tcctggctgt tgcggtggcg gcaagctggg taagcctatc cctaaccctc tcctcggtct |
| 781 | cgattctacg agtgctgtta tcacaagttt gtacaaaaaa gctgaacgag aaacgtaaaa |
| 841 | tgatataaat atcaatatat taaattagat tttgcataaa aaacagacta cataatactg |
| 901 | taaaacacaa catatccagt cactatggcg gccgcattag gcaccccagg ctttacactt |
| 961 | tatgcttccg gctcgtataa tgtgtggatt ttgagttagg atccggcgag attttcagga |
| 1021 | gctaaggaag ctaaaatgga gaaaaaaatc actggatata ccaccgttga tatatcccaa |
| 1081 | tggcatcgta aagaacattt tgaggcattt cagtcagttg ctcaatgtac ctataaccag |
| 1141 | accgttcagc tggatattac ggccttttta aagaccgtaa agaaaaataa gcacaagttt |
| 1201 | tatccggcct ttattcacat tcttgcccgc ctgatgaatg ctcatccgga attccgtatg |
| 1261 | gcaatgaaag acggtgagct ggtgatatgg gatagtgttc acccttgtta caccgrntc |
| 1321 | catgagcaaa ctgaaacgtt ttcatcgctc tggagtgaat accacgacga tftccggcag |
| 1381 | tttctacaca tatattcgca agatgtggcg tgttacggtg aaaacctggc ctatttccct |
| 1441 | aaagggttta ttgagaatat gtttttcgtc tcagccaatc cctgggtgag tttcaccagt |
| 1501 | tttgatttaa acgtggccaa tatggacaac ttcttcgccc ccgttttcac catgggcaaa |
| 1561 | tattatacgc aaggcgacaa ggtgctgatg ccgctggcga ttcaggttca tcatgccgtc |
| 1621 | tgtgatggct tccatgtcgg cagaatgctt aatgaattac aacagtactg cgatgagtgg |
| 1681 | cagggcgggg cgtaaacgcg tggatccggc ttactaaaag ccagataaca gtatgcgtat |
| 1741 | ttgcgcgcac cggtgctagc gtatacccga agtatgtcaa aaagaggtgt gctatgaagc |
| 1801 | agcgtattac agtgacagtt gacagcgaca gctatcagtt gctcaaggca tatatgatgt |
| 1861 | caatatctcc ggtctggtaa gcacaaccat gcagaatgaa gcccgtcgtc tgcgtgccga |
| 1921 | acgctggaaa gcggaaaatc aggaagggat ggctgaggtc gcccggttta ttgaaatgaa |
| 1981 | cggctctttt gctgacgaga acagggactg gtgaaatgca gtttaaggtt tacacctata |
| 2041 | aaagagagag ccgttatcgt ctgtttgtgg atgtacagag tgatattatt gacacgcccg |
| 2101 | ggcgacggat ggtgatcccc ctggccagtg cacgtctgct gtcagataaa gtctcccgtg |
| 2161 | aactttaccc ggtggtgcat atcggggatg aaagctggcg catgatgacc accgatatgg |
| 2221 | ccagtgtgcc ggtctccgtt atcggggaag aagtggctga tctcagccac cgcgaaaatg |
| 2281 | acatcaaaaa cgccattaac ctgatgttct ggggaatata aatgtcaggc tccgttatac |
| 2341 | acagccagtc tgcaggtcga ccatagtgac tggatatgtt gtgttttaca gtattatgta |
| 2401 | gtctgttttt tatgcaaaat ctaatttaat atattgatat ttatatcatt ttacgtttct |
| 2461 | cgttcagctt tcttgtacaa agtggtgata attaattaag ataacaccgg ttagtaatga |
| 2521 | gtttaaacgg gggaggctaa ctgaaacacg gaaggagaca ataccggaag gaacccgcgc |
| 2581 | tatgacggca ataaaaagac agaataaaac gcacgggtgt tgggtcgttt gttcataaac |
| 2641 | gcggggttcg gtcccagggc tggcactctg tcgatacccc accgagaccc cattggggcc |
| 2701 | aatacgcccg cgtttcttcc ttttccccac cccacccccc aagttcgggt gaaggcccag |
| 2761 | ggctcgcagc caacgtcggg gcggcaggcc ctgccatagc agatctgcgc agctggggct |
| 2821 | ctagggggta tccccacgcg ccctgtagcg gcgcattaag cgcggcgggt gtggtggtta |
| 2881 | cgcgcagcgt gaccgctaca cttgccagcg ccctagcgcc cgctcctttc gctttcttcc |
| 2941 | cttcctttct cgccacgttc gccggctttc cccgtcaagc tctaaatcgg ggcatccctt |
| 3001 | tagggttccg atttagtgct ttacggcacc tcgaccccaa aaaacttgat tagggtgatg |
| 3061 | gttcacgtag tgggccatcg ccctgataga cggtttttcg ccctttgacg ttggagtcca |
| 3121 | cgttctttaa tagtggactc ttgttccaaa ctggaacaac actcaaccct atctcggtct |
| 3181 | attcttttga tttataaggg attttgggga tttcggccta ttggttaaaa aatgagctga |
| 3241 | tttaacaaaa atttaacgcg aattaattct gtggaatgtg tgtcagttag ggtgtggaaa |
| 3301 | gtccccaggc tccccagcag gcagaagtat gcaaagcatg catctcaatt agtcagcaac |
| 3361 | caggtgtgga aagtccccag gctccccagc aggcagaagt atgcaaagca tgcatctcaa |
| 3421 | ttagtcagca accatagtcc cgcccctaac tccgcccatc ccgcccctaa ctccgcccag |
| 3481 | ttccgcccat tctccgcccc atggctgact aatttttttt atttatgcag aggccgaggc |
| 3541 | cgcctctgcc tctgagctat tccagaagta gtgaggaggc ttttttggag gcctaggctt |
| 3601 | ttgcaaaaag ctcccgggag cttgtatatc cattttcgga tctgatcagc acgtgttgac |
| 3661 | aattaatcat cggcatagta tatcggcata gtataatacg acaaggtgag gaactaaacc |
| 3721 | atggccaagc ctttgtctca agaagaatcc accctcattg aaagagcaac ggctacaatc |
| 3781 | aacagcatcc ccatctctga agactacagc gtcgccagcg cagctctctc tagcgacggc |
| 3841 | cgcatcttca ctggtgtcaa tgtatatcat tttactgggg gaccttgtgc agaactcgtg |
| 3901 | gtgctgggca ctgctgctgc tgcggcagct ggcaacctga cttgtatcgt cgcgatcgga |
| 3961 | aatgagaaca ggggcatctt gagcccctgc ggacggtgcc gacaggtgct tctcgatctg |
| 4021 | catcctggga tcaaagccat agtgaaggac agtgatggac agccgacggc agttgggaft |
| 4081 | cgtgaattgc tgccctctgg ttatgtgtgg gagggctaag cacttcgtgg ccgaggagca |
| 4141 | ggactgacac gtgctacgag atttcgattc caccgccgcc ttctatgaaa ggttgggctt |
| 4201 | cggaatcgtt ttccgggacg ccggctggat gatcctccag cgcggggatc tcatgctgga |
| 4261 | gttcttcgcc caccccaact tgtttattgc agcttataat ggttacaaat aaagcaatag |
| 4321 | catcacaaat ttcacaaata aagcattttt ttcactgcat tctagttgtg gtttgtccaa |
| 4381 | actcatcaat gtatcttatc atgtctgtat accgtcgacc tctagctaga gcttggcgta |
| 4441 | atcatggtca tagctgtttc ctgtgtgaaa ttgttatccg ctcacaattc cacacaacat |
| 4501 | acgagccgga agcataaagt gtaaagcctg gggtgcctaa tgagtgagct aactcacatt |
| 4561 | aattgcgttg cgctcactgc ccgctttcca gtcgggaaac ctgtcgtgcc agctgcatta |
| 4621 | atgaatcggc caacgcgcgg ggagaggcgg tttgcgtatt gggcgctctt ccgcttcctc |
| 4681 | gctcactgac tcgctgcgct cggtcgttcg gctgcggcga gcggtatcag ctcactcaaa |
| 4741 | ggcggtaata cggttatcca cagaatcagg ggataacgca ggaaagaaca tgtgagcaaa |
| 4801 | aggccagcaa aaggccagga accgtaaaaa ggccgcgttg ctggcgtttt tccataggct |
| 4861 | ccgcccccct gacgagcatc acaaaaatcg acgctcaagt cagaggtggc gaaacccgac |
| 4921 | aggactataa agataccagg cgtttccccc tggaagctcc ctcgtgcgct ctcctgttcc |
| 4981 | gaccctgccg cttaccggat acctgtccgc ctttctccct tcgggaagcg tggcgctttc |
| 5041 | tcatagctca cgctgtaggt atctcagttc ggtgtaggtc gttcgctcca agctgggctg |
| 5101 | tgtgcacgaa ccccccgttc agcccgaccg ctgcgcctta tccggtaact atcgtcttga |
| 5161 | gtccaacccg gtaagacacg acttatcgcc actggcagca gccactggta acaggattag |
| 5221 | cagagcgagg tatgtaggcg gtgctacaga gttcttgaag tggtggccta actacggcta |
| 5281 | cactagaaga acagtatttg gtatctgcgc tctgctgaag ccagttacct tcggaaaaag |
| 5341 | agttggtagc tcttgatccg gcaaacaaac caccgctggt agcggttttt ttgtttgcaa |
| 5401 | gcagcagatt acgcgcagaa aaaaaggatc tcaagaagat cctttgatct tttctacggg |
| 5461 | gtctgacgct cagtggaacg aaaactcacg ttaagggatt ttggtcatga gattatcaaa |
| 5521 | aaggatcttc acctagatcc ttttaaatta aaaatgaagt tttaaatcaa tctaaagtat |
| 5581 | atatgagtaa acttggtctg acagttacca atgcttaatc agtgaggcac ctatctcagc |
| 5641 | gatctgtcta tttcgttcat ccatagttgc ctgactcccc gtcgtgtaga taactacgat |
| 5701 | acgggagggc ttaccatctg gccccagtgc tgcaatgata ccgcgagacc cacgctcacc |
| 5761 | ggctccagat ttatcagcaa taaaccagcc agccggaagg gccgagcgca gaagtggtcc |
| 5821 | tgcaacttta tccgcctcca tccagtctat taattgttgc cgggaagcta gagtaagtag |
| 5881 | ttcgccagtt aatagtttgc gcaacgttgt tgccattgct acaggcatcg tggtgtcacg |
| 5941 | ctcgtcgttt ggtatggctt cattcagctc cggttcccaa cgatcaaggc gagttacatg |
| 6001 | atcccccatg ttgtgcaaaa aagcggttag ctccttcggt cctccgatcg ttgtcagaag |
| 6061 | taagttggcc gcagtgttat cactcatggt tatggcagca ctgcataatt ctcttactgt |
| 6121 | catgccatcc gtaagatgct tttctgtgac tggtgagtac tcaaccaagt cattctgaga |
| 6181 | atagtgtatg cggcgaccga gttgctcttg cccggcgtca atacgggata ataccgcgcc |
| 6241 | acatagcaga actttaaaag tgctcatcat tggaaaacgt tcttcggggc gaaaactctc |
| 6301 | aaggatctta ccgctgttga gatccagttc gatgtaaccc actcgtgcac ccaactgatc |
| 6361 | ttcagcatct tttactttca ccagcgtttc tgggtgagca aaaacaggaa ggcaaaatgc |
| 6421 | cgcaaaaaag ggaataaggg cgacacggaa atgttgaata ctcatactct tcctttttca |
| 6481 | atattattga agcatttatc agggttattg tctcatgagc ggatacatat ttgaatgtat |
| 6541 | ttagaaaaat aaacaaatag gggttccgcg cacatttccc cgaaaagtgc cacctgacgt |
| 6601 | cgacggatcg ggagatctcc cgatccccta tggtgcactc tcagtacaat ctgctctgat |
| 6661 | gccgcatagt taagccagta tctgctccct gcttgtgtgt tggaggtcgc tgagtagtgc |
| 6721 | gcgagcaaaa tttaagctac aacaaggcaa ggcttgaccg acaattgcat gaagaatctg |
| 6781 | cttagggtta ggcgttttgc gctgcttcg |

TABLE 39

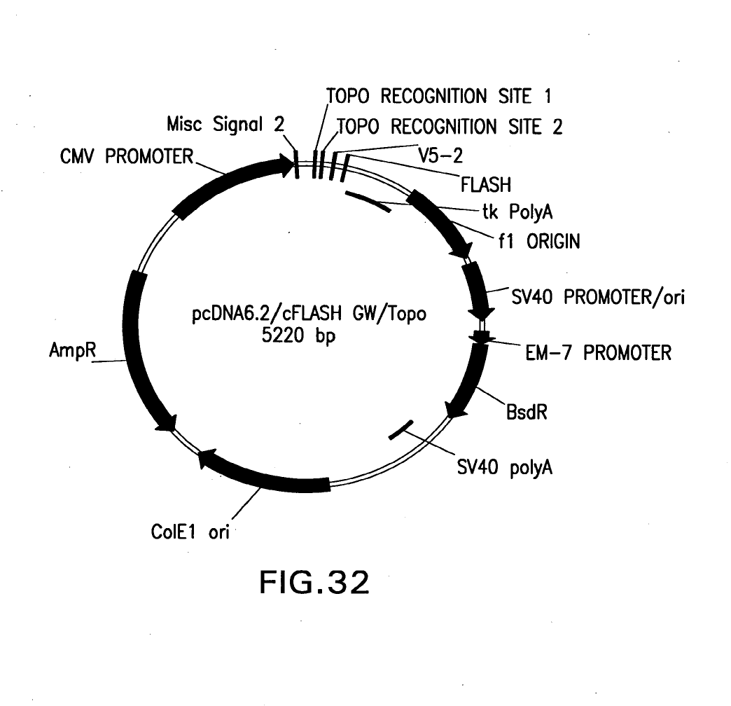

Nucleotide Sequence of pcDNA6.2/cFLASH GW/TOPO
(See Fig. 32) (SEQ ID NO: 161)

| | |
|---|---|
| 1 | cgatgtacgg gccagatata cgcgttgaca ttgattattg actagttatt aatagtaatc |
| 61 | aattacgggg tcattagttc atagcccata tatggagttc cgcgttacat aacttacggt |
| 121 | aaatggcccg cctggctgac cgcccaacga cccccgccca ttgacgtcaa taatgacgta |
| 181 | tgttcccata gtaacgccaa tagggacttt ccattgacgt caatgggtgg agtatttacg |
| 241 | gtaaactgcc cacttggcag tacatcaagt gtatcatatg ccaagtacgc cccctattga |
| 301 | cgtcaatgac ggtaaatggc ccgcctggca ttatgcccag tacatgacct tatgggactt |
| 361 | tcctacttgg cagtacatct acgtattagt catcgctatt accatggtga tgcggttttg |
| 421 | gcagtacatc aatgggcgtg gatagcggtt tgactcacgg ggatttccaa gtctccaccc |
| 481 | cattgacgtc aatgggagtt tgttttggca ccaaaatcaa cgggactttc caaaatgtcg |
| 541 | taacaactcc gccccattga cgcaaatggg cggtaggcgt gtacggtggg aggtctatat |
| 601 | aagcagagct ctctggctaa ctagagaacc cactgcttac tggcttatcg aaattaatac |
| 661 | gactcactat agggagaccc aagctggcta gttaagctga gcatcaacaa gtttgtacaa |
| 721 | aaaagcaggc tccgcggccg cccccttcac cgacattttg tttaaacttt ggtacctgga |
| 781 | tcctttaaac gcgtggatcc ggcttactaa aagccagata acagtatgcg tatttgcgcg |
| 841 | ctgatttttg cggtataaga atatatactg atatgtatac ccgaagtatg tcaaaaagag |
| 901 | gtgtgctatg aagcagcgta ttacagtgac agttgacagc gacagctatc agttgctcaa |
| 961 | ggcatatatg atgtcaatat ctccggtctg gtaagcacaa ccatgcagaa tgaagcccgt |
| 1021 | cgtctgcgtg ccgaacgctg gaaagcggaa aatcaggaag ggatggctga ggtcgcccgg |
| 1081 | tttattgaaa tgaacggctc ttttgctgac gagaacaggg actggtgaaa tgcagtttaa |
| 1141 | ggfttacacc tataaaagag agagccgtta tcgtctgttt gtggatgtac agagtgatat |
| 1201 | tattgacacg cccgggcgac ggatggtgat ccccctggcc agtgcacgtc tgctgtcaga |
| 1261 | taaagtctcc cgtgaacttt acccggtggt gcatatcggg gatgaaagct ggcgcatgat |
| 1321 | gaccaccgat atggccagtg tgccggtctc cgttatcggg gaagaagtgg ctgatctcag |
| 1381 | ccaccgcgaa aatgacatca aaaacgccat taacctgatg ttctggggaa tataattaaa |
| 1441 | ggatccaggt accaaagttt aaacaaaatg tcaagggtgg gcgcgccgac ccagctttct |
| 1501 | tgtacaaagt ggttgatgct gttaacggga agcctatccc taaccctctc ctcggtctcg |
| 1561 | attctacgcg taccggtgct ggtggctgtt gtcctggctg ttgcggtggc ggctagtaat |
| 1621 | gagtttaaac gggggaggct aactgaaaca cggaaggaga caataccgga aggaacccgc |
| 1681 | gctatgacgg caataaaaag acagaataaa acgcacgggt gttgggtcgt ttgttcataa |
| 1741 | acgcggggtt cggtcccagg gctggcactc tgtcgatacc ccaccgagac cccattgggg |
| 1801 | ccaatacgcc cgcgtttctt ccttttcccc accccacccc ccaagttcgg gtgaaggccc |
| 1861 | agggctcgca gccaacgtcg gggcggcagg ccctgccata gcagatctgc gcagctgggg |
| 1921 | ctctaggggg tatccccacg cgccctgtag cggcgcatta agcgcggcgg gtgtggtggt |
| 1981 | tacgcgcagc gtgaccgcta cacttgccag cgccctagcg cccgctcctt tcgctttctt |
| 2041 | cccttccttt ctcgccacgt tcgccggctt tccccgtcaa gctctaaatc ggggcatccc |
| 2101 | tttagggttc cgatttagtg ctttacggca cctcgacccc aaaaaacttg attagggtga |
| 2161 | tggttcacgt agtgggccat cgccctgata gacggttttt cgccctttga cgttggagtc |
| 2221 | cacgttcfft aatagtggac tcttgttcca aactggaaca acactcaacc ctatctcggt |
| 2281 | ctattctttt gatttataag ggattttggg gatttcggcc tattggttaa aaaatgagct |
| 2341 | gatttaacaa aaatttaacg cgaattaatt ctgtggaatg tgtgtcagtt agggtgtgga |
| 2401 | aagtccccag gctccccagc aggcagaagt atgcaaagca tgcatctcaa ttagtcagca |
| 2461 | accaggtgtg gaaagtcccc aggctcccca gcaggcagaa gtatgcaaag catgcatctc |
| 2521 | aattagtcag caaccatagt cccgccccta actccgccca tcccgcccct aactccgccc |
| 2581 | agttccgccc attctccgcc ccatggctga ctaatttttt ttatttatgc agaggccgag |
| 2641 | gccgcctctg cctctgagct attccagaag tagtgaggag gcttttttgg aggcctaggc |
| 2701 | ttttgcaaaa agctcccggg agcttgtata tccattttcg gatctgatca gcacgtgttg |
| 2761 | acaattaatc atcggcatag tatatcggca tagtataata cgacaaggtg aggaactaaa |
| 2821 | ccatggccaa gcctttgtct caagaagaat ccaccctcat tgaaagagca acggctacaa |
| 2881 | tcaacagcat ccccatctct gaagactaca gcgtcgccag cgcagctctc tctagcgacg |
| 2941 | gccgcatctt cactggtgtc aatgtatatc attttactgg gggaccttgt gcagaactcg |
| 3001 | tggtgctggg cactgctgct gctgcggcag ctggcaacct gacttgtatc gtcgcgatcg |
| 3061 | gaaatgagaa caggggcatc ttgagcccct gcggacggtg ccgacaggtg cttctcgatc |
| 3121 | tgcatcctgg gatcaaagcc atagtgaagg acagtgatgg acagccgacg gcagttggga |
| 3181 | ttcgtgaatt gctgccctct ggttatgtgt gggagggcta agcacttcgt ggccgaggag |
| 3241 | caggactgac acgtgctacg agatttcgat tccaccgccg ccttctatga aaggttgggc |
| 3301 | ttcggaatcg ttttccggga cgccggctgg atgatcctcc agcgcgggga tctcatgctg |
| 3361 | gagttcttcg cccaccccaa cttgtttatt gcagcttata atggttacaa ataaagcaat |
| 3421 | agcatcacaa atttcacaaa taaagcattt ttttcactgc attctagttg tggtttgtcc |
| 3481 | aaactcatca atgtatctta tcatgtctgt ataccgtcga cctctagcta gagcttggcg |
| 3541 | taatcatggt catagctgtt tcctgtgtga aattgttatc cgctcacaat tccacacaac |
| 3601 | atacgagccg gaagcataaa gtgtaaagcc tggggtgcct aatgagtgag ctaactcaca |
| 3661 | ttaattgcgt tgcgctcact gcccgctttc cagtcgggaa acctgtcgtg ccagctgcat |
| 3721 | taatgaatcg gccaacgcgc ggggagaggc ggtttgcgta ttgggcgctc ttccgcttcc |
| 3781 | tcgctcactg actcgctgcg ctcggtcgtt cggctgcggc gagcggtatc agctcactca |
| 3841 | aaggcggtaa tacggttatc cacagaatca ggggataacg caggaaagaa catgtgagca |
| 3901 | aaaggccagc aaaaggccag gaaccgtaaa aaggccgcgt tgctggcgtt tttccatagg |
| 3961 | ctccgccccc ctgacgagca tcacaaaaat cgacgctcaa gtcagaggtg gcgaaacccg |
| 4021 | acaggactat aaagatacca ggcgtttccc cctggaagct ccctcgtgcg ctctcctgtt |
| 4081 | ccgaccctgc cgcttaccgg atacctgtcc gcctttctcc cttcgggaag cgtggcgctt |
| 4141 | tctcatagct cacgctgtag gtatctcagt tcggtgtagg tcgttcgctc caagctgggc |
| 4201 | tgtgtgcacg aaccccccgt tcagcccgac cgctgcgcct tatccggtaa ctatcgtctt |
| 4261 | gagtccaacc cggtaagaca cgacttatcg ccactggcag cagccactgg taacaggatt |
| 4321 | agcagagcga ggtatgtagg cggtgctaca gagttcttga agtggtggcc taactacggc |
| 4381 | tacactagaa gaacagtatt tggtatctgc gctctgctga agccagttac cttcggaaaa |
| 4441 | agagttggta gctcttgatc cggcaaacaa accaccgctg gtagcggttt ttttgtttgc |
| 4501 | aagcagcaga ttacgcgcag aaaaaaagga tctcaagaag atcctttgat cttttctacg |
| 4561 | gggtctgacg ctcagtggaa cgaaaactca cgttaaggga ttttggtcat gagattatca |
| 4621 | aaaaggatct tcacctagat ccttttaaat taaaaatgaa gttttaaatc aatctaaagt |
| 4681 | atatatgagt aaacttggtc tgacagttac caatgcttaa tcagtgaggc acctatctca |
| 4741 | gcgatctgtc tatttcgttc atccatagtt gcctgactcc ccgtcgtgta gataactacg |
| 4801 | atacgggagg gcttaccatc tggccccagt gctgcaatga taccgcgaga cccacgctca |
| 4861 | ccggctccag atttatcagc aataaaccag ccagccggaa gggccgagcg cagaagtggt |
| 4921 | cctgcaactt tatccgcctc catccagtct attaattgtt gccgggaagc tagagtaagt |
| 4981 | agttcgccag ttaatagttt gcgcaacgtt gttgccattg ctacaggcat cgtggtgtca |
| 5041 | cgctcgtcgt ttggtatggc ttcattcagc tccggttccc aacgatcaag gcgagttaca |
| 5101 | tgatccccca tgttgtgcaa aaaagcggtt agctccttcg gtcctccgat cgttgtcaga |
| 5161 | agtaagttgg ccgcagtgtt atcactcatg gttatggcag cactgcataa ttctcttact |
| 5221 | gtcatgccat ccgtaagatg cttttctgtg actggtgagt actcaaccaa gtcattctga |
| 5281 | gaatagtgta tgcggcgacc gagttgctct tgcccggcgt caatacggga taataccgcg |
| 5341 | ccacatagca gaactttaaa agtgctcatc attggaaaac gttcttcggg gcgaaaactc |
| 5401 | tcaaggatct taccgctgtt gagatccagt tcgatgtaac ccactcgtgc acccaactga |
| 5461 | tcttcagcat cttttacttt caccagcgtt tctgggtgag caaaaacagg aaggcaaaat |
| 5521 | gccgcaaaaa agggaataag ggcgacacgg aaatgttgaa tactcatact cttcctfftt |
| 5581 | caatattatt gaagcattta tcagggttat tgtctcatga gcggatacat atttgaatgt |
| 5641 | atttagaaaa ataaacaaat aggggttccg cgcacatttc cccgaaaagt gccacctgac |
| 5701 | gtcgacggat cgggagatct cccgatcccc tatggtgcac tctcagtaca atctgctctg |
| 5761 | atgccgcata gttaagccag tatctgctcc ctgcttgtgt gttggaggtc gctgagtagt |
| 5821 | gcgcgagcaa aatttaagct acaacaaggc aaggcttgac cgacaattgc atgaagaatc |
| 5881 | tgcttagggt taggcgtttt gcgctgcttc g |

TABLE 40

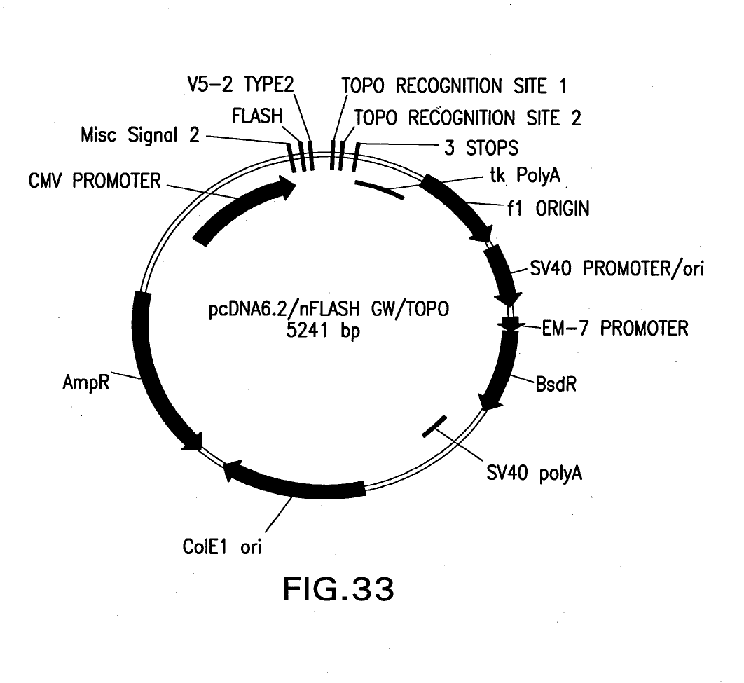

Nucleotide Sequence of pcDNA6.2/nFLASH GW/TOPO
(See FIG. 33) (SEQ ID NO: 162)

| | |
|---|---|
| 1 | cgatgtacgg gccagatata cgcgttgaca ttgattattg actagttatt aatagtaatc |
| 61 | aattaegggg teattagttc atageceata tatggagttc cgcgttacat aacttacggt |
| 121 | aaatggcccg cctggctgac cgcccaacga cccccgccca ttgacgtcaa taatgacgta |
| 181 | tgttcccata gtaacgecaa tagggacttt ccattgacgt eaatgggtgg agtatttaeg |
| 241 | gtaaactgcc cacttggcag tacatcaagt gtatcatatg ccaagtacgc cccctattga |
| 301 | cgtcaatgae ggtaaatgge cegcctggca ttatgcccag tacatgaeet tatgggaett |
| 361 | tcctacttgg cagtacatct acgtattagt catcgctatt accatggtga tgcggttttg |
| 421 | gcagtac ate aatgggcgtg gatagcggtt tgactcacgg ggatttccaa gtctccaccc |
| 481 | cattgaegtc aatgggagtt tgttttggea ccaaaateaa egggaetttc caaaatgtcg |
| 541 | taacaactcc gccccattga cgcaaatggg cggtaggcgt gtacggtggg aggtctatat |
| 601 | aageagaget etctggetaa etagagaacc cactgettac tggcttatcg aaattaatac |
| 661 | gacteactat agggagaccc aagetggcta gttaagctgc aceatggctg gtggctgttg |
| 721 | tcctggctgt tgcggtggcg gcaagctggg taagcctatc cctaaccctc tcctcggtct |
| 781 | cgattetacg agtgetgtta teaeaagttt gtacaaaaaa gcaggctccg eggeegecce |
| 841 | cttcaccgac attttgttta aactttggta ectggatcct ttaaacgcgt ggatccggct |
| 901 | tactaaaagc cagataacag tatgegtatt tgegegetga tttttgeggt ataagaatat |
| 961 | ataetgatat gtatacccga agtatgtcaa aaagaggtgt gctatgaagc agcgtattac |
| 1021 | agtgacagtt gacagcgaca gctatcagtt gcteaaggca tatatgatgt caatatetec |
| 1081 | ggtctggtaa gcacaaccat geagaatgaa gcccgtcgtc tgcgtgccga acgctggaaa |
| 1141 | gcggaaaatc aggaagggat ggctgaggtc gcccggttta ttgaaatgaa cggctctttt |
| 1201 | gctgacgaga acagggactg gtgaaatgca gtttaaggtt tacacetata aaagagagag |
| 1261 | ccgttatcgt etgtttgtgg atgtacagag tgatattatt gacacgcccg ggcgacggat |
| 1321 | ggtgatcccc etggccagtg eaegtctget gtcagataaa gtctcccgtg aactttaccc |
| 1381 | ggtggtgcat atcggggatg aaagctggcg catgatgacc accgatatgg ccagtgtgcc |
| 1441 | ggtctccgtt atcggggaag aagtggctga tctcagccac cgcgaaaatg acatcaaaaa |
| 1501 | cgccattaac ctgatgttct ggggaatata attaaaggat ccaggtacca aagtttaaac |
| 1561 | aaaatgtcaa gggtgggcgc gccgacccag ctttcttgta caaagtggtg ataattaatt |
| 1621 | aagataacac cggttagtaa tgagtttaaa cgggggaggc taactgaaac acggaaggag |
| 1681 | acaataccgg aaggaacccg cgctatgacg gcaataaaaa gacagaataa aacgcacggg |
| 1741 | tgttgggtcg tttgttcata aacgcggggt tcggtcccag ggctggcact ctgtcgatac |
| 1801 | cccaccgaga ccccattggg gccaatacgc ccgcgtttct tccttttccc caccccaccc |
| 1861 | cccaagttcg ggtgaaggcc cagggctcgc agccaacgtc ggggcggcag gccctgccat |
| 1921 | agcagatctg cgcagctggg gctctagggg gtatccccac gcgccctgta gcggcgcatt |
| 1981 | aagcgcggcg ggtgtggtgg ttacgcgcag cgtgaccgct acacttgcca gcgccctagc |
| 2041 | gcccgctcct ttcgctttct tcccttcctt tctcgccacg ttcgccggct ttccccgtca |
| 2101 | agctctaaat cggggcatcc ctttagggtt ccgatttagt gctttacggc acctcgaccc |
| 2161 | caaaaaactt gattagggtg atggttcacg tagtgggcca tcgccctgat agacggtttt |
| 2221 | tcgccctttg acgttggagt ccacgttctt taatagtgga ctcttgttcc aaactggaac |
| 2281 | aacactcaac cctatctcgg tctattcttt tgatttataa gggattttgg ggatttcggc |
| 2341 | ctattggtta aaaaatgagc tgatttaaca aaaatttaac gcgaattaat tctgtggaat |
| 2401 | gtgtgtcagt tagggtgtgg aaagtcccca ggctccccag caggcagaag tatgcaaagc |
| 2461 | atgcatctca attagtcagc aaccaggtgt ggaaagtccc caggctcccc agcaggcaga |
| 2521 | agtatgcaaa gcatgcatct caattagtca gcaaccatag tcccgcccct aactccgccc |
| 2581 | atcccgcccc taactccgcc cagttccgcc cattctccgc cccatggctg actaattttt |
| 2641 | tttatttatg cagaggccga ggccgcctct gcctctgagc tattccagaa gtagtgagga |
| 2701 | ggcttttttg gaggcctagg cttttgcaaa aagctcccgg gagcttgtat atccattttc |
| 2761 | ggatctgatc agcacgtgft gacaattaat catcggcata gtatatcggc atagtataat |
| 2821 | acgacaaggt gaggaactaa accatggcca agcctttgtc tcaagaagaa tccaccctca |
| 2881 | ttgaaagagc aacggctaca atcaacagca tccccatctc tgaagactac agcgtcgcca |
| 2941 | gcgcagctct ctctagcgac ggccgcatct tcactggtgt caatgtatat cattttactg |
| 3001 | ggggaccttg tgcagaactc gtggtgctgg gcactgctgc tgctgcggca gctggcaacc |
| 3061 | tgacttgtat cgtcgcgatc ggaaatgaga acaggggcat cttgagcccc tgcggacggt |
| 3121 | gccgacaggt gcttctcgat ctgcatcctg ggatcaaagc catagtgaag gacagtgatg |
| 3181 | gacagccgac ggcagttggg attcgtgaat tgctgccctc tggttatgtg tgggagggct |
| 3241 | aagcacttcg tggccgagga gcaggactga cacgtgctac gagatttcga ttccaccgcc |
| 3301 | gccttctatg aaaggttggg cttcggaatc gttttccggg acgccggctg gatgatcctc |
| 3361 | cagcgcgggg atctcatgct ggagttcttc gcccacccca acttgtttat tgcagcttat |
| 3421 | aatggttaca aataaagcaa tagcatcaca aatttcacaa ataaagcatt tttttcactg |
| 3481 | cattctagtt gtggtttgtc caaactcatc aatgtatctt atcatgtctg tataccgtcg |
| 3541 | acctctagct agagcttggc gtaatcatgg tcatagctgt ttcctgtgtg aaattgttat |
| 3601 | ccgctcacaa ttccacacaa catacgagcc ggaagcataa agtgtaaagc ctggggtgcc |
| 3661 | taatgagtga gctaactcac attaattgcg ttgcgctcac tgcccgcttt ccagtcggga |
| 3721 | aacctgtcgt gccagctgca ttaatgaatc ggccaacgcg cggggagagg cggtttgcgt |
| 3781 | attgggcgct cttccgcttc ctcgctcact gactcgctgc gctcggtcgt tcggctgcgg |
| 3841 | cgagcggtat cagctcactc aaaggcggta atacggffat ccacagaatc aggggataac |
| 3901 | gcaggaaaga acatgtgagc aaaaggccag caaaaggcca ggaaccgtaa aaaggccgcg |
| 3961 | ttgctggcgt ttttccatag gctccgcccc cctgacgagc atcacaaaaa tcgacgctca |
| 4021 | agtcagaggt ggcgaaaccc gacaggacta taaagatacc aggcgtttcc ccctggaagc |
| 4081 | tccctcgtgc gctctcctgt tccgaccctg ccgcttaccg gatacctgtc cgcctttctc |
| 4141 | ccttcgggaa gcgtggcgct ttctcatagc tcacgctgta ggtatctcag ttcggtgtag |
| 4201 | gtcgttcgct ccaagctggg ctgtgtgcac gaaccccccg ttcagcccga ccgctgcgcc |
| 4261 | ttatccggta actatcgtct tgagtccaac ccggtaagac acgacttatc gccactggca |
| 4321 | gcagccactg gtaacaggat tagcagagcg aggtatgtag gcggtgctac agagttcttg |
| 4381 | aagtggtggc ctaactacgg ctacactaga agaacagtat ttggtatctg cgctctgctg |
| 4441 | aagccagtta ccttcggaaa aagagttggt agctcttgat ccggcaaaca aaccaccgct |
| 4501 | ggtagcggtt tttttgtttg caagcagcag attacgcgca gaaaaaaagg atctcaagaa |
| 4561 | gatcctttga tcttttctac ggggtctgac gctcagtgga acgaaaactc acgttaaggg |
| 4621 | attttggtca tgagattatc aaaaaggatc ttcacctaga tccttttaaa ttaaaaatga |
| 4681 | agttttaaat caatctaaag tatatatgag taaacttggt ctgacagtta ccaatgctta |
| 4741 | atcagtgagg cacctatctc agcgatctgt ctatttcgtt catccatagt tgcctgactc |
| 4801 | cccgtcgtgt agataactac gatacgggag ggcttaccat ctggccccag tgctgcaatg |
| 4861 | ataccgcgag acccacgctc accggctcca gatttatcag caataaacca gccagccgga |
| 4921 | agggccgagc gcagaagtgg tcctgcaact ttatccgcct ccatccagtc tattaattgt |
| 4981 | tgccgggaag ctagagtaag tagttcgcca gttaatagft tgcgcaacgt tgttgccatt |
| 5041 | gctacaggca tcgtggtgtc acgctcgtcg tttggtatgg cttcattcag ctccggttcc |
| 5101 | caacgatcaa ggcgagttac atgatccccc atgttgtgca aaaaagcggt tagctccttc |
| 5161 | ggtcctccga tcgttgtcag aagtaagttg gccgcagtgt tatcactcat ggttatggca |
| 5221 | gcactgcata attctcttac tgtcatgcca tccgtaagat gcttttctgt gactggtgag |
| 5281 | tactcaacca agtcattctg agaatagtgt atgcggcgac cgagttgctc ttgcccggcg |
| 5341 | tcaatacggg ataataccgc gccacatagc agaactttaa aagtgctcat cattggaaaa |
| 5401 | cgttcttcgg ggcgaaaact ctcaaggatc ttaccgctgt tgagatccag ttcgatgtaa |
| 5461 | cccactcgtg cacccaactg atcttcagca tcttttactt tcaccagcgt ttctgggtga |
| 5521 | gcaaaaacag gaaggcaaaa tgccgcaaaa aagggaataa gggcgacacg gaaatgttga |
| 5581 | atactcatac tcttcctttt tcaatattat tgaagcattt atcagggtta ttgtctcatg |
| 5641 | agcggataca tatttgaatg tatttagaaa aataaacaaa taggggttcc gcgcacattt |
| 5701 | ccccgaaaag tgccacctga cgtcgacgga tcgggagatc tcccgatccc ctatggtgca |
| 5761 | ctctcagtac aatctgctct gatgccgcat agttaagcca gtatctgctc cctgcttgtg |
| 5821 | tgttggaggt cgctgagtag tgcgcgagca aaatttaagc tacaacaagg caaggcttga |
| 5881 | ccgacaattg catgaagaat ctgcttaggg ttaggcgttt tgcgctgctt cg |

TABLE 41

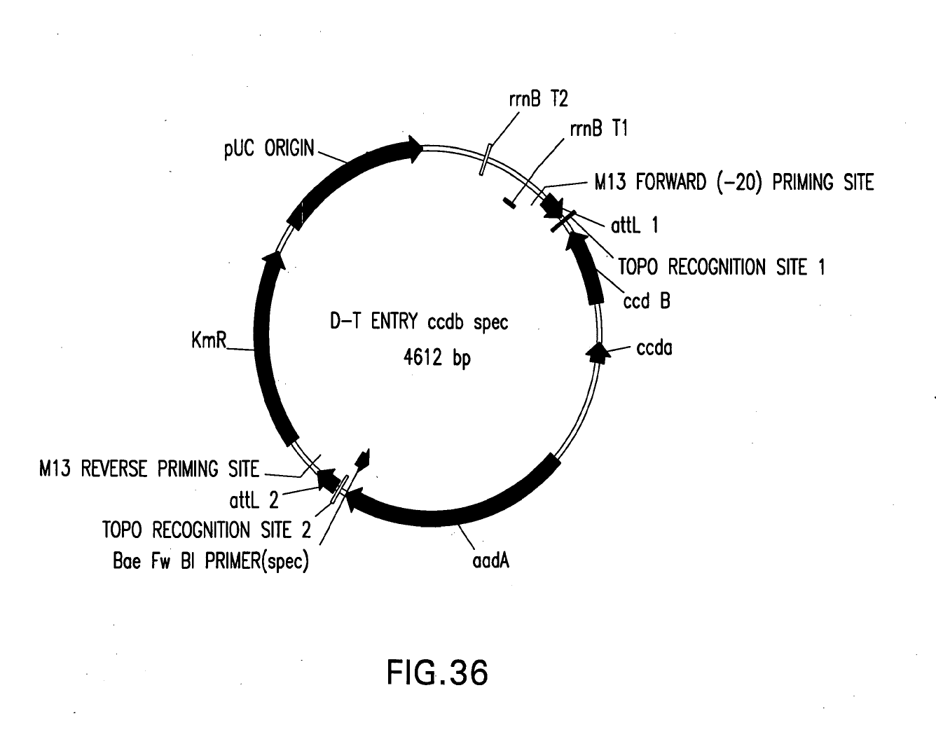

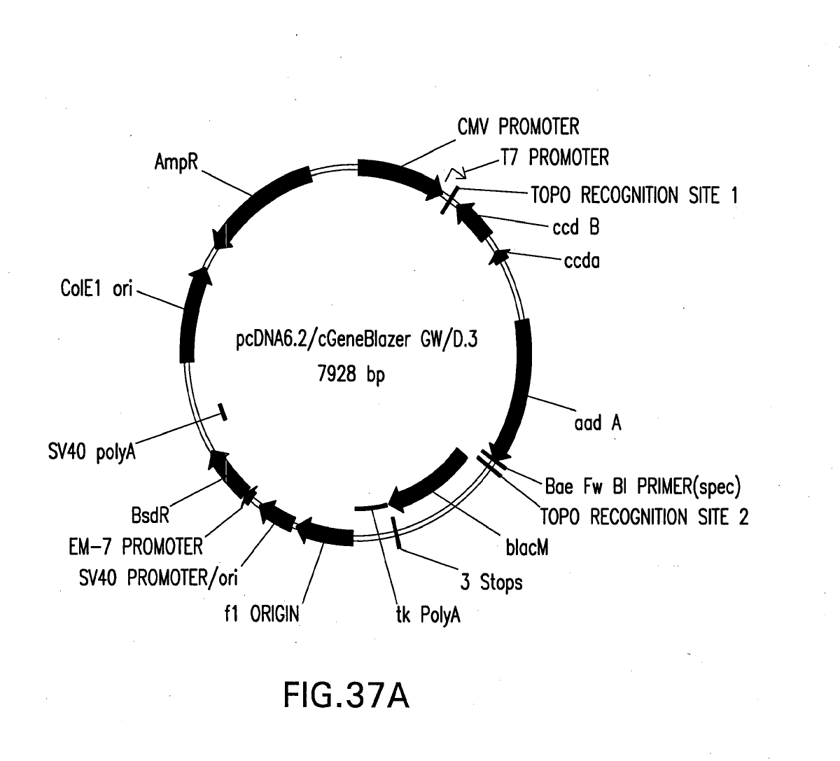

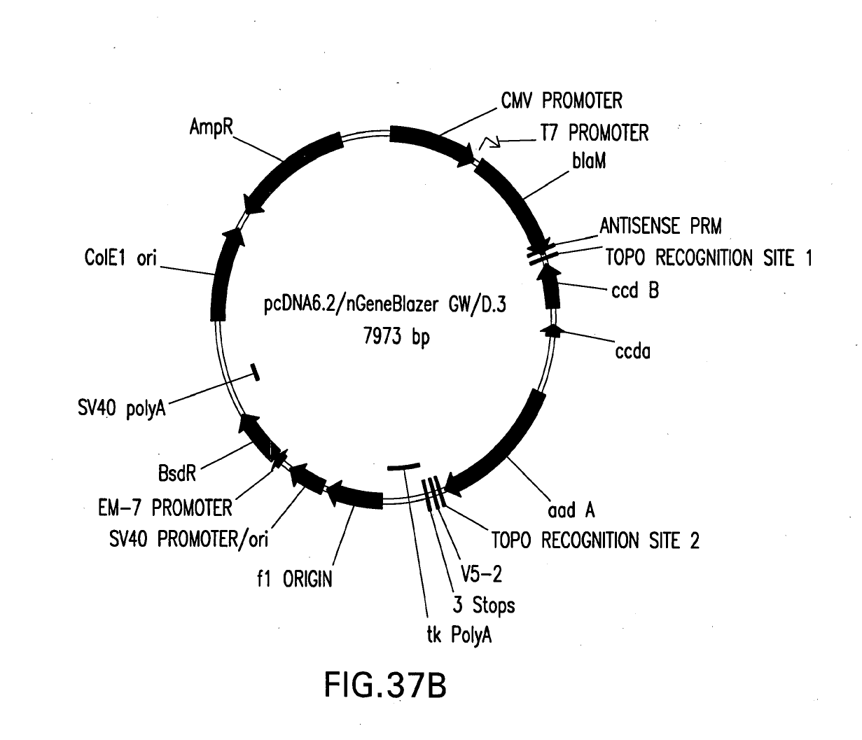

Nucleotide Sequence of D-T Entry ccdb spec
(See FIG. 36) (SEQ ID NO: 163)

| | |
|---|---|
| 1 | gtaaaacgac ggccagtctt aagctcgggc cccaaataat gattttattt tgactgatag |
| 61 | tgacctgttc gttgcaacaa attgatgagc aatgcttttt tataatgcca actttgtaca |
| 121 | aaaaagcagg ctccgcggcc gcccccttga catttttgtt taaactttgg tacctggatc |
| 181 | ctttaattat attccccaga acatcaggtt aatggcgttt ttgatgtcat tttcgcggtg |
| 241 | gctgagatca gccacttctt ccccgataac ggagaccggc acactggcca tatcggtggt |
| 301 | catcatgcgc cagctttcat ccccgatatg caccaccggg taaagttcac gggagacttt |
| 361 | atctgacagc agacgtgcac tggccagggg gatcaccatc cgtcgcccgg gcgtgtcaat |
| 421 | aatatcactc tgtacatcca caaacagacg ataacggctc tctcttttat aggtgtaaac |
| 481 | cttaaactgc atttcaccag cccctgttct cgtcagcaaa agagccgttc atttcaataa |
| 541 | accgggcgac ctcagccatc ccttcctgat tttccgcttt ccagcgttcg gcacgcagac |
| 601 | gacgggcttc attctgcatg gttgtgctta ccagaccgga gatattgaca tcatatatgc |
| 661 | cttgagcaac tgatagctgt cgctgtcaac tgtcactgta atacgctgct tcatagcata |
| 721 | cctctttttg acatacttcg ggtatacata tcagtatata ttcttatacc gcaaaaatca |
| 781 | gcgcgcaaat acgcatactg ttatctggct tttagtaagc cggatccacg cgtttacgcc |
| 841 | ccgccctgcc actcatcgca gtactgttgt aattcattaa gcattctgcc gacatggaag |
| 901 | ccatcacaaa cggcatgatg aacctgaatc gccagcggca tcagcacctt gtcgccttgc |
| 961 | gtataatatt tgcccatgaa acgaattcgc ccttcggagt actaggacag aaatgcctcg |
| 1021 | acttcgctgc tgcccaaggt tgccgggtga cgcacaccgt ggaaacggat gaaggcacga |
| 1081 | acccagtgga cataagcctg ttcggttcgt aagctgtaat gcaagtagcg tatgcgctca |
| 1141 | cgcaactggt ccagaacctt gaccgaacgc agcggtggta acggcgcagt ggcggttttc |
| 1201 | atggcttgtt atgactgttt ttttggggta cagtctatgc ctcgggcatc caagcagcaa |
| 1261 | gcgcgttacg ccgtgggtcg atgtttgatg ttatggagca gcaacgatgt tacgcagcag |
| 1321 | ggcagtcgcc ctaaaacaaa gttaaacatc atgagggaag cggtgatcgc cgaagtatcg |
| 1381 | actcaactat cagaggtagt tggcgtcatc gagcgccatc tcgaaccgac gttgctggcc |
| 1441 | gtacatttgt acggctccgc agtggatggc ggcctgaagc cacacagtga tattgatttg |
| 1501 | ctggttacgg tgaccgtaag gcttgatgaa acaacgcggc gagctttgat caacgacctt |
| 1561 | ttggaaactt cggcttcccc tggagagagc gagattctcc gcgctgtaga agtcaccatt |
| 1621 | gftgtgcacg acgacatcat tccgtggcgt tatccagcta agcgcgaact gcaatttgga |
| 1681 | gaatggcagc gcaatgacat tcttgcaggt atcttcgagc cagccacgat cgacattgat |
| 1741 | ctggctatct tgctgacaaa agcaagagaa catagcgttg ccttggtagg tccagcggcg |
| 1801 | gaggaactct ttgatccggt tcctgaacag gatctatttg aggcgctaaa tgaaacctta |
| 1861 | acgctatgga actcgccgcc tgactgggct ggcgatgagc gaaatgtagt gcttacgttg |
| 1921 | tcccgcattt ggtacagcgc agtaaccggc aaaatcgcgc cgaaggatgt cgctgccgac |
| 1981 | tgggcaatgg agcgcctgcc ggcccagtat cagcccgtca tacttgaagc tagacaggct |
| 2041 | tatcttggac aagaagaaga tcgcttggcc tcgcgcgcag atcagttgga agaatttgtc |
| 2101 | cactacgtga aaggcgagat caccaaggta gtcggcaaat aattaaagga tccaggtacc |
| 2161 | aaagtttaaa caaaaatgtc aagggtgggc gcgccgaccc agctttcttg tacaaagttg |
| 2221 | gcattataag aaagcattgc ttatcaattt gttgcaacga acaggtcact atcagtcaaa |
| 2281 | ataaaatcat tatttgccat ccagctgata tcccctatag tgagtcgtat tacatggtca |
| 2341 | tagctgtttc ctggcagctc tggcccgtgt ctcaaaatct ctgatgttac attgcacaag |
| 2401 | ataaaaatat atcatcatga acaataaaac tgtctgctta cataaacagt aatacaaggg |
| 2461 | gtgttatgag ccatattcaa cgggaaacgt cgaggccgcg attaaaffcc aacatggatg |
| 2521 | ctgatttata tgggtataaa tgggctcgcg ataatgtcgg gcaatcaggt gcgacaatct |
| 2581 | atcgcttgta tgggaagccc gatgcgccag agttgtttct gaaacatggc aaaggtagcg |
| 2641 | ttgccaatga tgttacagat gagatggtca gactaaactg gctgacggaa tttatgcctc |
| 2701 | ttccgaccat caagcatttt atccgtactc ctgatgatgc atggttactc accactgcga |
| 2761 | tccccggaaa aacagcattc caggtattag aagaatatcc tgattcaggt gaaaatattg |
| 2821 | ttgatgcgct ggcagtgttc ctgcgccggt tgcattcgat tcctgtttgt aattgtcctt |
| 2881 | ttaacagcga tcgcgtattt cgtctcgctc aggcgcaatc acgaatgaat aacggtttgg |
| 2941 | ttgatgcgag tgattttgat gacgagcgta atggctggcc tgttgaacaa gtctggaaag |
| 3001 | aaatgcataa acttttgcca ttctcaccgg attcagtcgt cactcatggt gatttctcac |
| 3061 | ttgataacct tatttttgac gaggggaaat taataggttg tattgatgtt ggacgagtcg |
| 3121 | gaatcgcaga ccgataccag gatcttgcca tcctatggaa ctgcctcggt gagttttctc |
| 3181 | cttcattaca gaaacggctt tttcaaaaat atggtattga taatcctgat atgaataaat |
| 3241 | tgcagtttca tttgatgctc gatgagtttt tctaatcaga attggttaat tggttgtaac |
| 3301 | actggcagag cattacgctg acttgacggg acggcgcaag ctcatgacca aaatccctta |
| 3361 | acgtgagtta cgcgtcgttc cactgagcgt cagaccccgt agaaaagatc aaaggatctt |
| 3421 | cttgagatcc tttttttctg cgcgtaatct gctgcttgca aacaaaaaaa ccaccgctac |
| 3481 | cagcggtggt ttgtttgccg gatcaagagc taccaactct ttttccgaag gtaactggct |
| 3541 | tcagcagagc gcagatacca aatactgtcc ttctagtgta gccgtagtta ggccaccact |
| 3601 | tcaagaactc tgtagcaccg cctacatacc tcgctctgct aatcctgtta ccagtggctg |
| 3661 | ctgccagtgg cgataagtcg tgtcttaccg ggttggactc aagacgatag ttaccggata |
| 3721 | aggcgcagcg gtcgggctga acggggggtt cgtgcacaca gcccagcttg gagcgaacga |
| 3781 | cctacaccga actgagatac ctacagcgtg agcattgaga aagcgccacg cttcccgaag |
| 3841 | ggagaaaggc ggacaggtat ccggtaagcg gcagggtcgg aacaggagag cgcacgaggg |
| 3901 | agcttccagg gggaaacgcc tggtatcttt atagtcctgt cgggtttcgc cacctctgac |
| 3961 | ttgagcgtcg atttttgtga tgctcgtcag gggggcggag cctatggaaa aacgccagca |
| 4021 | acgcggcctt tttacggttc ctggcctttt gctggccttt tgctcacatg ttctttcctg |
| 4081 | cgttatcccc tgattctgtg gataaccgta ttaccgcctt tgagtgagct gataccgctc |
| 4141 | gccgcagccg aacgaccgag cgcagcgagt cagtgagcga ggaagcggaa gagcgcccaa |
| 4201 | tacgcaaacc gcctctcccc gcgcgttggc cgattcatta atgcagctgg cacgacaggt |
| 4261 | ttcccgactg gaaagcgggc agtgagcgca acgcaattaa tacgcgtacc gctagccagg |
| 4321 | aagagtttgt agaaacgcaa aaaggccatc cgtcaggatg gccttctgct tagtttgatg |
| 4381 | cctggcagtt tatggcgggc gtcctgcccg ccaccctccg ggccgttgct tcacaacgtt |
| 4441 | caaatccgct cccggcggat ttgtcctact caggagagcg ttcaccgaca aacaacagat |
| 4501 | aaaacgaaag gcccagtctt ccgactgagc ctttcgtttt atttgatgcc tggcagttcc |
| 4561 | ctactctcgc gttaacgcta gcatggatgt tttcccagtc acgacgtt |

Having now fully described the present invention in some detail by way of illustration and example for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims

All publications, patents and patent applications mentioned in this specification are indicative of the level of skill of those skilled in the art to which this invention pertains, and are herein incorporated by reference to the same extent as if each individual publication, patent or patent application was specifically and individually indicated to be incorporated by reference.

1. An isolated nucleic acid molecule comprising a nucleotide sequence encoding a polypeptide having a detectable activity and further comprising at least one of: (a) one or more recombination sites; and (b) one or more topoisomerase recognition sites and/or one or more topoisomerases; wherein the nucleotide sequence encoding a polypeptide having a detectable activity is not operably linked to a promoter sequence.

2. The nucleic acid molecule of claim 1, wherein said nucleic acid molecule is a circular molecule.

3. The nucleic acid molecule of claim 1, wherein said nucleic acid molecule comprises two or more recombination sites.

4. The nucleic acid molecule of claim 3, wherein at least one of said two or more recombination sites flanks each end of a topoisomerase recognition site in said molecule.

5. The nucleic acid molecule of claim 1, wherein said polypeptide having a detectable activity is an enzyme.

6. The nucleic acid molecule of claim 5, wherein said enzyme is an enzyme having β-lactamase activity.

7. The nucleic acid molecule of claim 6, wherein said enzyme having β-lactamase activity is a cytoplasmic β-lactamase.

8. The nucleic acid molecule of claim 3, wherein said recombination sites are selected from the group consisting of:
(a) attB,
(b) attP,
(c) attL,
(d) attR,
(e) lox sites,
(f) psi sites,
(g) dif sites,
(h) cer sites,
(i) frt sites,
and mutants, variants, and derivatives of the recombination sites of (a), (b), (c), (d), (e), (f), (g), (h) or (i) which retain the ability to undergo recombination.

9-18. (canceled)

19. A method of joining at least a first nucleic acid molecule and a second nucleic acid molecule, said method comprising:
(a) contacting a first nucleic acid molecule which comprises (i) at least a first nucleotide sequence encoding a polypeptide having a detectable activity, and (ii) at least one topoisomerase site and/or topoisomerase, with at least a second nucleic acid molecule; wherein the nucleotide sequence encoding a polypeptide having a detectable activity is not operably linked to a promoter sequence; and
(b) incubating said first and second nucleic acid molecules under conditions sufficient to join said first and second nucleic acid molecules.

20. The method according to claim 19, wherein the second nucleic acid molecule comprises a nucleotide sequence to be assayed for promoter activity.

21. The method according to claim 19, wherein the first nucleic acid molecule further comprises one or more recombination sites.

22. The method according to claim 21, wherein the first nucleic acid molecule comprises two recombination sites that do not recombine with each other.

23. The method according to claim 19, wherein the second nucleic acid molecule comprises one or more topoisomerase recognition sites and/or one or more topoisomerases and/or one or more recombination sites.

24. The method according to claim 19, wherein the second nucleic acid molecule comprises two recombination sites that do not recombine with each other.

25-28. (canceled)

29. A method of making a nucleic acid molecule, said method comprising:
(a) providing a first nucleic acid molecule comprising (i) a first nucleotide sequence encoding a polypeptide having a detectable activity, and (ii) at least a first recombination site, wherein the nucleotide sequence encoding a polypeptide having a detectable activity is not operably linked to a promoter sequence;
(b) providing a second nucleic acid molecule comprising a nucleotide sequence encoding a polypeptide of interest and at least a second recombination site; and
(c) forming a mixture in vitro between said first and second nucleic acid molecules and at least one recombination protein, under conditions sufficient to cause recombination in vitro between said first and second recombination sites, thereby producing a third nucleic acid molecule comprising a third nucleotide sequence that encodes all or a portion of the polypeptide having a detectable activity and all or a portion of the polypeptide of interest in the same reading frame and comprising a third recombination site that is the product of the recombination of the first and second recombination sites.

30. The method according to claim 29, wherein the third recombination site is located between the nucleotide sequence encoding a polypeptide having a detectable activity and the nucleotide sequence encoding a polypeptide of interest.

31. The method according to claim 30, further comprising expressing a polypeptide from the third nucleic acid molecule.

32. The method according to claim 31, wherein the polypeptide is a fusion protein comprising all or a portion of the amino acid sequence of the polypeptide having a detectable activity, all or a portion of the amino acid sequence of the polypeptide interest, and at least one amino acid encoded by the third recombination site.

33-36. (canceled)

37. A kit comprising the isolated nucleic acid molecule of claim 1.

38. The kit of claim 37, further comprising one or more components selected from the group consisting of one or more topoisomerases, one or more recombination proteins, one or more vectors, one or more polypeptides having polymerase activity, and one or more host cells.

39-44. (canceled)


SEQUENCE LISTING (257 sequences)

SEQ ID NO: 1 - DNA, 211 bp, Artificial Sequence
  TOPO cloning site of pGeneBLAzer-TOPO
gagatctaatacgactcactatagggagacccaagctggctagcgtttaaacttaagcttggtaccgagctcggatccactagtccagtgtggtggaattgcccttaagggcaattcgcccttcaccatggacccagaaacgctggtgaaagtaaaagatgctgaagataagcattggtaagataaacgggggaggctaactgaaacacgg

SEQ ID NO: 2 - protein, 17 aa, Artificial Sequence
  Synthetic Construct
MDPETLVKVKDAEDKHW

SEQ ID NO: 3 - DNA, 283 bp, Artificial Sequence
  Recombination region of pcDNA 6.2/cGeneBLAzer-DEST
caaatgggcggtaggcgtgtacggtgggaggtctatataagcagagctctctggctaactagagaacccactgcttactggcttatcgaaattaatacgactcactatagggagacccaagctggctagttaagctgagcatcaacaagtttgtacaaaaaagcaggctacccagctttcttgtacaaagtggtcttgtacaaagtggttgatgctgttatggacccagaaacgctggtgaaagtaaaagatgctgaagataagcattggtaaccggttagta

SEQ ID NO: 4 - protein, 33 aa, Artificial Sequence
  Recombination region of pcDNA 6.2/cGeneBLAzer-DEST
PAFLYKVVLYKVVDAVMDPETLVKVKDAEDKHW

SEQ ID NO: 5 - DNA, 367 bp, Artificial Sequence
  Recombination region of pcDNA 6.2/nGeneBLAzer-DEST
caaatgggcggtaggcgtgtacggtgggaggtctatataagcagagctctctggctaactagagaacccactgcttactggcttatcgaaattaatacgactcactatagggagacccaagctggctagttaagctgagcatggacccagaaacgctggtgaaagtaaaagatgctgaagataagcattggctgttatcaacaagtttgtacaaaaaagcaggctacccagctttcttgtacaaagtggttgataacgggaagcctatccctaaccctctcctcggtctcgattctacgcgtaccggttagtaatgagtttaaacgggggaggctaactgaaacacggaaggagacaataccggaag

SEQ ID NO: 6 - protein, 56 aa, Artificial Sequence
  Recombination region of pcDNA 6.2/nGeneBLAzer-DEST
MDPETLVKVKDAEDKHWLLSTSLYKKAGYPAFLYKVVDNGKPIPNPLLGLDSTRTG

SEQ ID NO: 7 - DNA, 19 bp, Artificial Sequence
  Oligonucleotide useful for TOPO-adapting nucleic acid moelcules
ccttgacatagtacagttg

SEQ ID NO: 8 - DNA, 19 bp, Artificial Sequence
  Oligonucleotide useful for TOPO-adapting nucleic acid moelcules
caccgacatagtacagttg

SEQ ID NO: 9 - DNA, 31 bp, Artificial Sequence
  Oligonucleotide useful for TOPO-adapting nucleic acid moelcules
ggccgcccccttcaccgacatagtacagttg

SEQ ID NO: 10 - DNA, 52 bp, Artificial Sequence
  Oligonucleotide useful for TOPO-adapting nucleic acid moelcules
ggccgccttgtttaactttaagaaggagcccttcaccgacatagtacagttg

SEQ ID NO: 11 - DNA, 9 bp, Artificial Sequence
  Oligonucleotide useful for TOPO-adapting nucleic acid moelcules
aagggtggg

SEQ ID NO: 12 - DNA, 28 bp, Artificial Sequence
  Oligonucleotide useful for TOPO-adapting nucleic acid moelcules
cgcgcccacccttgacatagtacagttg

SEQ ID NO: 13 - DNA, 12 bp, Artificial Sequence
  Oligonucleotide useful for TOPO-adapting nucleic acid moelcules
ggtgaagggggc

SEQ ID NO: 14 - DNA, 31 bp, Artificial Sequence
  Oligonucleotide useful for TOPO-adapting nucleic acid moelcules
ggccgcccccttcaccgactatgtacagttg

SEQ ID NO: 15 - DNA, 33 bp, Artificial Sequence
  Oligonucleotide useful for TOPO-adapting nucleic acid moelcules
ggtgaagggctccttcttaaagttaaacaaggc

SEQ ID NO: 16 - DNA, 52 bp, Artificial Sequence
  Oligonucleotide useful for TOPO-adapting nucleic acid moelcules
ggccgccttgtttaactttaagaaggagcccttcaccgactatgtacagttg

SEQ ID NO: 17 - DNA, 12 bp, Artificial Sequence
  Oligonucleotide useful for TOPO-adapting nucleic acid moelcules
ctgtactatgtc

SEQ ID NO: 18 - protein, 264 aa, Artificial Sequence
  Polypeptide having beta-lactamase activity
MDPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHW

SEQ ID NO: 19 - DNA, 7437 bp, Artificial Sequence
  Nucleotide sequence of pET160-DEST
gatctcgatcccgcgaaattaatacgactcactataggggaattgtgagcggataacaattcccctctagaaataattttgtttaactttaagaaggagatatacatatgcatcatcaccatcaccatggtgctggtggctgttgtcctggctgttgcggtggcggcgaaaacctgtattttcagggaattatcacaagtttgtacaaaaaagctgaacgagaaacgtaaaatgatataaatatcaatatattaaattagattttgcataaaaaacagactacataatactgtaaaacacaacatatccagtcactatggcggccgcattaggcaccccaggctttacactttatgcttccggctcgtataatgtgtggattttgagttaggatccggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaaacgcgtggatccggcttactaaaagccagataacagtatgcgtatttgcgcgcaccggtgctagcgtatacccgaagtatgtcaaaaagaggtgtgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacagggactggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccgccgcgaaaatgacatcaaaaacgccattaacctgatgttctggggaatataaatgtcaggctcccttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggtgataattaattaagatcagatccggctgctaacaaagcccgaaaggaagctgagttggctgctgccaccgctgagcaataactagcataaccccttggggcctctaaacgggtcttgaggggttttttgctgaaaggaggaactatatccggatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagctagcttatcgatgataagctgtcaaacatgagaattaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcga

SEQ ID NO: 20 - protein, 28 aa, Artificial Sequence
  Nucleotide sequence of pET160-DEST
MHHHHHHGAGGCCPGCCGGGENLYFQGI

SEQ ID NO: 21 - DNA, 7480 bp, Artificial Sequence
  Nucleotide Sequence of pET161-DEST
agatctcgatcccgcgaaattaatacgactcactataggggaattgtgagcggataacaattcccctctagaaataattttgtttaactttaagaaggagatatacatatggctagcatgactggtggacagcaaatgggtattatgattatcacaagtttgtacaaaaaagctgaacgagaaacgtaaaatgatataaatatcaatatattaaattagattttgcataaaaaacagactacataatactgtaaaacacaacatatccagtcactatggcggccgcattaggcaccccaggctttacactttatgcttccggctcgtataatgtgtggattttgagttaggatcctgcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaacgcgtggatccggcttactaaaagccagataacagtatgcgtatttgcgcgctgatttttgcggtataagaatatatactgatatgtatacccgaagtatgtcaaaaagaggtgtgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacagggactggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctcccttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggtgatcaattcgaagcttgaagctggtggctgttgtcctggctgttgcggtggcggcaccggtcatcatcaccatcaccattgagtttgatccggctgctaacaaagcccgaaaggaagctgagttggctgctgccaccgctgagcaataactagcataaccccttggggcctctaaacgggtcttgaggggttttttgctgaaaggaggaactatatccggatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcg

SEQ ID NO: 22 - protein, 15 aa, Artificial Sequence
  Sequence of pET161-DEST
MASMTGGQQMGIMII

SEQ ID NO: 23 - protein, 219 aa, Artificial Sequence
  Sequence of pET161-DEST
MEKKITGYTTVDISQWHRKEHFEAFQSVAQCTYNQTVQLDITAFLKTVKKNKHKFYPAFIHILARLMNAHPEFRMAMKDGELVIWDSVHPCYTVFHEQTETFSSLWSEYHDDFRQFLHIYSQDVACYGENLAYFPKGFIENMFFVSANPWVSFTSFDLNVANMDNFFAPVFTMGKYYTQGDKVLMPLAIQVHHAVCDGFHVGRMLNELQQYCDEWQGGA

SEQ ID NO: 24 - protein, 101 aa, Artificial Sequence
  Sequence of pET161-DEST
MQFKVYTYKRESRYRLFVDVQSDIIDTPGRRMVIPLASARLLSDKVSRELYPVVHIGDESWRMMTTDMASVPVSVIGEEVADLSHRENDIKNAINLMFWGI

SEQ ID NO: 25 - protein, 26 aa, Artificial Sequence
  Sequence of pET161-DEST
INSKLEAGGCCPGCCGGGTGHHHHHH

SEQ ID NO: 26 - protein, 286 aa, Artificial Sequence [sequence omitted]

SEQ ID NO: 27 - protein, 35 aa, Artificial Sequence
  Sequence of pET161-DEST
ASSSLSFSRSVNVWLLIKRAMLRAVFSCLVTDASV

SEQ ID NO: 28 - protein, 28 aa, Artificial Sequence
  Sequence of pET161-DEST
GGFLFMGVMIPMKRERMLTIRVTDDEHA

SEQ ID NO: 29 - protein, 11 aa, Artificial Sequence
  Sequence of pET161-DEST
VFLFTSETGNS

SEQ ID NO: 30 - protein, 7 aa, Artificial Sequence
  Nucleotide Sequence of pET161-DEST
LPFTAWP

SEQ ID NO: 31 - protein, 26 aa, Artificial Sequence
  Sequence of pET161-DEST
ESCSKRSTLVCPSRRKSCLMVVNGGI

SEQ ID NO: 32 - protein, 33 aa, Artificial Sequence
  Sequence of pET161-DEST
HELSSVSSYPTTEISAPTRSPDSVMARIAPSAI

SEQ ID NO: 33 - protein, 20 aa, Artificial Sequence
  Sequence of pET161-DEST
SLATSIAVGTMPSFSICMVC

SEQ ID NO: 34 - protein, 15 aa, Artificial Sequence
  Sequence of pET161-DEST
KPDMALQSPSRSAIG

SEQ ID NO: 35 - protein, 29 aa, Artificial Sequence
  Sequence of pET161-DEST
LRVRYLCQPARRRRAETELNGPANSAICW

SEQ ID NO: 36 - protein, 52 aa, Artificial Sequence
  Sequence of pET161-DEST
PNATRCSTPSRVPSSWEKIILLMGVWSETSRNNAGTLVQAASTAMASWSSSG

SEQ ID NO: 37 - protein, 36 aa, Artificial Sequence
  Sequence of pET161-DEST
LMISPLTRCARRLCTAALQASTPLRSTIDTTTLAPS

SEQ ID NO: 38 - protein, 41 aa, Artificial Sequence
  Sequence of pET161-DEST
SARDLIAATICDGACRARLEVATPISNDCLPASCCATRLGM

SEQ ID NO: 39 - protein, 28 aa, Artificial Sequence
  Sequence of pET161-DEST
FSSAIAASTFSRVFAETWLAWFTTRETV

SEQ ID NO: 40 - protein, 21 aa, Artificial Sequence
  Sequence of pET161-DEST
ETPAYSATSYNVTGFTFTTLN

SEQ ID NO: 41 - protein, 30 aa, Artificial Sequence
  Sequence of pET161-DEST
LSSGRYHAIPRKVLRHSMVSGISTLSLMRL

SEQ ID NO: 42 - DNA, 6562 bp, Artificial Sequence
  plasmid pET160/D-TOPO
tcgatcccgcgaaattaatacgactcactataggggaattgtgagcggataacaattcccctctagaaataattttgtttaactttaagaaggagatatacatatgcatcatcaccatcaccatggtgctggtggctgttgtcctggctgttgcggtggcggcgaaaacctgtattttcagggaattatcacaagtttgtacaaaaaagcaggctccgcggccgcccccttcaccgacatttttgtttaaactttggtacctggatcctttaaacgcgtggatccggcttactaaaagccagataacagtatgcgtatttgcgcgctgatttttgcggtataagaatatatactgatatgtatacccgaagtatgtcaaaaagaggtgtgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacagggactggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtgtgatgttctggggaatataattaaaggatccaggtaccaaagtttaaacaaaaatgtcaagggtgggcgcgccgacccagctttcttgtacaaataattaattaagatcagatccggctgctaacaaagcccgaaaggaagctgagttggctgctgccaccgctgagcaataactagcataaccccttggggcagtggtggctgatctcagccaccgcgaaaatgacatcaaaaacgccattaaccctctaaacgggtcttgaggggttttttgctgaaaggaggaactatatccggatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagctagcttatcgatgataagctgtcaaacatgagaattaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatc

SEQ ID NO: 43 - protein, 42 aa, Artificial Sequence
  plasmid pET160/D-TOPO
MHHHHHHGAGGCCPGCCGGGENLYFQGIITSLYKKAGSAAAP

SEQ ID NO: 44 - protein, 35 aa, Artificial Sequence; identical to SEQ ID NO: 27
  plasmid pET160/D-TOPO

SEQ ID NO: 45 - protein, 28 aa, Artificial Sequence
  plasmid pET160/D-TOPO
GGFLFMGVMIPMKRERMLTIRVTDDEHA

SEQ ID NO: 46 - protein, 11 aa, Artificial Sequence
  plasmid pET160/D-TOPO
VFLFTSETGNS

SEQ ID NO: 47 - protein, 7 aa, Artificial Sequence
  Synthetic Construct
LPFTAWP

SEQ ID NO: 48 - protein, 26 aa, Artificial Sequence
  plasmid pET160/D-TOPO
ESCSKRSTLVCPSRRKSCLMVVNGGI

SEQ ID NO: 49 - protein, 33 aa, Artificial Sequence; identical to SEQ ID NO: 32
  plasmid pET160/D-TOPO

SEQ ID NO: 50 - protein, 20 aa, Artificial Sequence
  plasmid pET160/D-TOPO
SLATSIAVGTMPSFSICMVC

SEQ ID NO: 51 - protein, 15 aa, Artificial Sequence
  plasmid pET160/D-TOPO
KPDMALQSPSRSAIG

SEQ ID NO: 52 - protein, 29 aa, Artificial Sequence
  plasmid pET160/D-TOPO
LRVRYLCQPARRRRAETELNGPANSAICW

SEQ ID NO: 53 - protein, 52 aa, Artificial Sequence; identical to SEQ ID NO: 36
  plasmid pET160/D-TOPO

SEQ ID NO: 54 - protein, 36 aa, Artificial Sequence; identical to SEQ ID NO: 37
  plasmid pET160/D-TOPO

SEQ ID NO: 55 - protein, 41 aa, Artificial Sequence; identical to SEQ ID NO: 38
  plasmid pET160/D-TOPO

SEQ ID NO: 56 - protein, 28 aa, Artificial Sequence
  plasmid pET160/D-TOPO
FSSAIAASTFSRVFAETWLAWFTTRETV

SEQ ID NO: 57 - protein, 21 aa, Artificial Sequence
  plasmid pET160/D-TOPO
ETPAYSATSYNVTGFTFTTLN

SEQ ID NO: 58 - protein, 30 aa, Artificial Sequence; identical to SEQ ID NO: 41
  plasmid pET160/D-TOPO

SEQ ID NO: 59 - DNA, 6584 bp, Artificial Sequence
  plasmid pET161/D-TOPO
agatctcgatcccgcgaaattaatacgactcactataggggaattgtgagcggataacaattcccctctagaaataattttgtttaactttaagaaggagatatacatatggctagcatgactggtggacagcaaatgggtattatgattatcacaagtttgtacaaaaaagcaggctccgcggccgcccccttcaccgacatttttgtttaaactttggtacctggatcctttaaacgcgtggatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataattaaaggatccaggtaccaaagtttaaacaaaaatgtcaagggtgggcgcgccgacccagctttcttgtacaaagtggtgatcaattcgaagcttgaagctggtggctgttgtcctggctgttgcggtggcggcaccggtcatcatcaccatcaccattgagtttgatccggctgctaacaaagcccgaaaggaagctgagttggctgctgccaccgctgagcaataactagcataaccccttggggcctctaaacgggtcttgaggggttttttgctgaaaggaggaactatatccggatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcg

SEQ ID NO: 60 - protein, 28 aa, Artificial Sequence
  plasmid pET161/D-TOPO
MASMTGGQQMGIMIITSLYKKAGSAAAP

SEQ ID NO: 61 - protein, 20 aa, Artificial Sequence
  plasmid pET161/D-TOPO
CIPEVCQKEVCYEAAYYSDS

SEQ ID NO: 62 - protein, 63 aa, Artificial Sequence
  plasmid pET161/D-TOPO
QRQLSVAQGIYDVNISGLVSTTMQNEARRLRAERWKAENQEGMAEVARFIEMNGSFADENRGW

SEQ ID NO: 63 - protein, 4 aa, Artificial Sequence
  plasmid pET161/D-TOPO
GLHL

SEQ ID NO: 64 - protein, 13 aa, Artificial Sequence
  plasmid pET161/D-TOPO
KREPLSSVCGCTE

SEQ ID NO: 65 - protein, 18 aa, Artificial Sequence
  plasmid pET161/D-TOPO
HARATDGDPPGQCTSAVR

SEQ ID NO: 66 - protein, 9 aa, Artificial Sequence
  plasmid pET161/D-TOPO
TLPGGAYRG

SEQ ID NO: 67 - protein, 22 aa, Artificial Sequence
  plasmid pET161/D-TOPO
KLAHDDHRYGQCAGLRYRGRSG

SEQ ID NO: 68 - protein, 6 aa, Artificial Sequence
  plasmid pET161/D-TOPO
SQPPRK

SEQ ID NO: 69 - protein, 5 aa, Artificial Sequence
  plasmid pET161/D-TOPO
HQKRH

SEQ ID NO: 70 - protein, 60 aa, Artificial Sequence
  plasmid pET161/D-TOPO
PDVLGNIIKGSRYQSLNKNVKGGRADPAFLYKVVINSKLEAGGCCPGCCGGGTGHHHHHH

SEQ ID NO: 71 - protein, 286 aa, Artificial Sequence [sequence omitted]

SEQ ID NO: 72 - protein, 35 aa, Artificial Sequence; identical to SEQ ID NO: 27
  plasmid pET161/D-TOPO

SEQ ID NO: 73 - protein, 28 aa, Artificial Sequence
  plasmid pET161/D-TOPO
GGFLFMGVMIPMKRERMLTIRVTDDEHA

SEQ ID NO: 74 - protein, 11 aa, Artificial Sequence
  plasmid pET161/D-TOPO
VFLFTSETGNS

SEQ ID NO: 75 - protein, 7 aa, Artificial Sequence
  plasmid pET161/D-TOPO
LPFTAWP

SEQ ID NO: 76 - protein, 26 aa, Artificial Sequence
  plasmid pET161/D-TOPO
ESCSKRSTLVCPSRRKSCLMVVNGGI

SEQ ID NO: 77 - protein, 33 aa, Artificial Sequence; identical to SEQ ID NO: 32
  plasmid pET161/D-TOPO

SEQ ID NO: 78 - protein, 20 aa, Artificial Sequence
  plasmid pET161/D-TOPO
SLATSIAVGTMPSFSICMVC

SEQ ID NO: 79 - protein, 15 aa, Artificial Sequence
  plasmid pET161/D-TOPO
KPDMALQSPSRSAIG

SEQ ID NO: 80 - protein, 29 aa, Artificial Sequence
  plasmid pET161/D-TOPO
LRVRYLCQPARRRRAETELNGPANSAICW

SEQ ID NO: 81 - protein, 52 aa, Artificial Sequence; identical to SEQ ID NO: 36
  plasmid pET161/D-TOPO

SEQ ID NO: 82 - protein, 36 aa, Artificial Sequence; identical to SEQ ID NO: 37
  plasmid pET161/D-TOPO

SEQ ID NO: 83 - protein, 41 aa, Artificial Sequence; identical to SEQ ID NO: 38
  plasmid pET161/D-TOPO

SEQ ID NO: 84 - protein, 28 aa, Artificial Sequence
  plasmid pET161/D-TOPO
FSSAIAASTFSRVFAETWLAWFTTRETV

SEQ ID NO: 85 - protein, 21 aa, Artificial Sequence
  plasmid pET161/D-TOPO
ETPAYSATSYNVTGFTFTTLN

SEQ ID NO: 86 - protein, 30 aa, Artificial Sequence; identical to SEQ ID NO: 41
  plasmid pET161/D-TOPO

SEQ ID NO: 87 - DNA, 6809 bp, Artificial Sequence
  plasmid pcDNA6.2/cFLASH-DEST
cgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagctgagcatcaacaagtttgtacaaaaaagctgaacgagaaacgtaaaatgatataaatatcaatatattaaattagattttgcataaaaaacagactacataatactgtaaaacacaacatatccagtcactatggcggccgcattaggcaccccaggctttacactttatgcttccggctcgtataatgtgtggattttgagttaggatccggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaaagatctggatccggcttactaaaagccagataacagtatgcgtatttgcgcgctgatttttgcggtataagaatatatactgatatgtatacccgaagtatgtcaaaaagaggtgtgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacagggactggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctccgttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggttgatgctgttaacgggaagcctatccctaaccctctcctcggtctcgattctacgcgtaccggtgctggtggctgttgtcctggctgttgcggtggcggctagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcg

SEQ ID NO: 88 - protein, 219 aa, Artificial Sequence; identical to SEQ ID NO: 23
  plasmid pcDNA6.2/cFLASH-DEST

SEQ ID NO: 89 - protein, 101 aa, Artificial Sequence; identical to SEQ ID NO: 24
  plasmid pcDNA6.2/cFLASH-DEST

SEQ ID NO: 90 - protein, 33 aa, Artificial Sequence
  plasmid pcDNA6.2/cFLASH-DEST
DAVNGKPIPNPLLGLDSTRTGAGGCCPGCCGGG

SEQ ID NO: 91 - protein, 132 aa, Artificial Sequence
  plasmid pcDNA6.2/cFLASH-DEST
MAKPLSQEESTLIERATATINSIPISEDYSVASAALSSDGRIFTGVNVYHFTGGPCAELVVLGTAAAAAAGNLTCIVAIGNENRGILSPCGRCRQVLLDLHPGIKAIVKDSDGQPTAVGIRELLPSGYVWEG

SEQ ID NO: 92 - protein, 98 aa, Artificial Sequence
  plasmid pcDNA6.2/cFLASH-DEST
EGLPSGPSAAMIPRDPRSPAPDLSAINQPAGRAERRSGPATLSASIQSINCCREARVSSSPVNSLRNVVAIATGIVVSRSSFGMASFSSGSQRSRRVT

SEQ ID NO: 93 - protein, 58 aa, Artificial Sequence
  plasmid pcDNA6.2/cFLASH-DEST
SPMLCKKAVSSFGPPIVVRSKLAAVLSLMVMAALHNSLTVMPSVRCFSVTGEYSTKSF

SEQ ID NO: 94 - protein, 50 aa, Artificial Sequence
  plasmid pcDNA6.2/cFLASH-DEST
CMRRPSCSCPASIRDNTAPHSRTLKVLIIGKRSSGRKLSRILPLLRSSSM

SEQ ID NO: 95 - protein, 6 aa, Artificial Sequence
  plasmid pcDNA6.2/cFLASH-DEST
PTRAPN

SEQ ID NO: 96 - protein, 12 aa, Artificial Sequence
  plasmid pcDNA6.2/cFLASH-DEST
SSASFTFTSVSG

SEQ ID NO: 97 - protein, 19 aa, Artificial Sequence
  plasmid pcDNA6.2/cFLASH-DEST
AKTGRQNAAKKGIRATRKC

SEQ ID NO: 98 - protein, 10 aa, Artificial Sequence
  plasmid pcDNA6.2/cFLASH-DEST
ILILFLFQYY

SEQ ID NO: 99 - protein, 17 aa, Artificial Sequence
  plasmid pcDNA6.2/cFLASH-DEST
SIYQGYCLMSGYIFECI

SEQ ID NO: 100 - protein, 7 aa, Artificial Sequence
  plasmid pcDNA6.2/cFLASH-DEST
KNKQIGV

SEQ ID NO: 101 - DNA, 6809 bp, Artificial Sequence
  plasmid pcDNA6.2/nFLASH-DEST
cgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagctgcaccatggctggtggctgttgtcctggctgttgcggtggcggcaagctgggtaagcctatccctaaccctctcctcggtctcgattctacgagtgctgttatcacaagtttgtacaaaaaagctgaacgagaaacgtaaaatgatataaatatcaatatattaaattagattttgcataaaaaacagactacataatactgtaaaacacaacatatccagtcactatggcggccgcattaggcaccccaggctttacactttatgcttccggctcgtataatgtgtggattttgagttaggatccggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaaacgcgtggatccggcttactaaaagccagataacagtatgcgtatttgcgcgcaccggtgctagcgtatacccgaagtatgtcaaaaagaggtgtgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacagggactggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctccgttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggtgataattaattaagataacaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcg

SEQ ID NO: 102 - protein, 34 aa, Artificial Sequence
  plasmid pcDNA6.2/nFLASH-DEST
MAGGCCPGCCGGGKLGKPIPNPLLGLDSTSAVIT

SEQ ID NO: 103 - protein, 219 aa, Artificial Sequence; identical to SEQ ID NO: 23
  plasmid pcDNA6.2/nFLASH-DEST

SEQ ID NO: 104 - protein, 101 aa, Artificial Sequence; identical to SEQ ID NO: 24
  plasmid pcDNA6.2/nFLASH-DEST

SEQ ID NO: 105 - protein, 4 aa, Artificial Sequence
  plasmid pcDNA6.2/nFLASH-DEST
DNTG

SEQ ID NO: 106 - protein, 132 aa, Artificial Sequence; identical to SEQ ID NO: 91
  plasmid pcDNA6.2/nFLASH-DEST

SEQ ID NO: 107 - protein, 98 aa, Artificial Sequence; identical to SEQ ID NO: 92
  plasmid pcDNA6.2/nFLASH-DEST

SEQ ID NO: 108 - protein, 58 aa, Artificial Sequence; identical to SEQ ID NO: 93
  plasmid pcDNA6.2/nFLASH-DEST

SEQ ID NO: 109 - protein, 50 aa, Artificial Sequence; identical to SEQ ID NO: 94
  plasmid pcDNA6.2/nFLASH-DEST

SEQ ID NO: 110 - protein, 6 aa, Artificial Sequence
  plasmid pcDNA6.2/nFLASH-DEST
PTRAPN

SEQ ID NO: 111 - protein, 12 aa, Artificial Sequence
  plasmid pcDNA6.2/nFLASH-DEST
SSASFTFTSVSG

SEQ ID NO: 112 - protein, 19 aa, Artificial Sequence
  plasmid pcDNA6.2/nFLASH-DEST
AKTGRQNAAKKGIRATRKC

SEQ ID NO: 113 - protein, 10 aa, Artificial Sequence
  plasmid pcDNA6.2/nFLASH-DEST
ILILFLFQYY

SEQ ID NO: 114 - protein, 17 aa, Artificial Sequence
  plasmid pcDNA6.2/nFLASH-DEST
SIYQGYCLMSGYIFECI

SEQ ID NO: 115 - protein, 7 aa, Artificial Sequence
  plasmid pcDNA6.2/nFLASH-DEST
KNKQIGV

SEQ ID NO: 116 - DNA, 7440 bp, Artificial Sequence
  plasmid pET-DEST151
gatctcgatcccgcgaaattaatacgactcactataggggaattgtgagcggataacaattcccctctagaaataattttgtttaactttaagaaggagatatacatatgcatcatcaccatcaccatggtaagcctatccctaaccctctcctcggtctcgattctacggaaaacctgtattttcagggaattatcacaagtttgtacaaaaaagctgaacgagaaacgtaaaatgatataaatatcaatatattaaattagattttgcataaaaaacagactacataatactgtaaaacacaacatatccagtcactatggcggccgcattaggcaccccaggctttacactttatgcttccggctcgtataatgtgtggattttgagttaggatccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaaacgcgtggatccggcttactaaaagccagataacagtatgcgtatttgcgcgcaccggtgctagcgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctcccttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggtgataattaattaagatcagatccggctgctaacaaagcccgaaaggaagctgagttggctgctgccaccgctgagcaataactagcataaccccttggggcctctaaacgggtcttgaggggttttttgctgaaaggaggaactatatccggatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagctagcttatcgatgataagctgtcaaacatgagaattaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcga

SEQ ID NO: 117 - protein, 29 aa, Artificial Sequence
  plasmid pET-DEST151
MHHHHHHGKPIPNPLLGLDSTENLYFQGI

SEQ ID NO: 118 - protein, 11 aa, Artificial Sequence
  plasmid pET-DEST151
VFLFTSETGNS

SEQ ID NO: 119 - protein, 7 aa, Artificial Sequence
  plasmid pET-DEST151
LPFTAWP

SEQ ID NO: 120 - protein, 26 aa, Artificial Sequence
  plasmid pET-DEST151
ESCSKRSTLVCPSRRKSCLMVVNGGI

SEQ ID NO: 121 - protein, 33 aa, Artificial Sequence; identical to SEQ ID NO: 32
  plasmid pET-DEST151

SEQ ID NO: 122 - protein, 20 aa, Artificial Sequence
  plasmid pET-DEST151
SLATSIAVGTMPSFSICMVC

SEQ ID NO: 123 - protein, 15 aa, Artificial Sequence
  plasmid pET-DEST151
KPDMALQSPSRSAIG

SEQ ID NO: 124 - protein, 29 aa, Artificial Sequence
  plasmid pET-DEST151
LRVRYLCQPARRRRAETELNGPANSAICW

SEQ ID NO: 125 - protein, 52 aa, Artificial Sequence; identical to SEQ ID NO: 36
  plasmid pET-DEST151

SEQ ID NO: 126 - protein, 36 aa, Artificial Sequence; identical to SEQ ID NO: 37
  plasmid pET-DEST151

SEQ ID NO: 127 - protein, 41 aa, Artificial Sequence; identical to SEQ ID NO: 38
  plasmid pET-DEST151

SEQ ID NO: 128 - protein, 28 aa, Artificial Sequence
  plasmid pET-DEST151
FSSAIAASTFSRVFAETWLAWFTTRETV

SEQ ID NO: 129 - protein, 21 aa, Artificial Sequence
  plasmid pET-DEST151
ETPAYSATSYNVTGFTFTTLN

SEQ ID NO: 130 - protein, 30 aa, Artificial Sequence; identical to SEQ ID NO: 41
  plasmid pET-DEST151

SEQ ID NO: 131 - DNA, 3303 bp, Artificial Sequence
  plasmid pENTR-DT.2 BaeIv.2 ccdB DT
ctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgctagcatggatgttttcccagtcacgacgttgtaaaacgacggccagtcttaagctcgggccccaaataatgattttattttgactgatagtgacctgttcgttgcaacaaattgatgagcaatgcttttttataatgccaactttgtacaaaaaagcaggctccgcggccgcccccttcaccgacatttttgtttaaactttggtacctggatcctttaaacgcgtggatccggcttactaaaagccagataacagtatgcgtatttgcgcgctgatttttgcggtataagaatatatactgatatgtatacccgaagtatgtcaaaaagaggtgtgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacagggactggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataattaaaggatccaggtaccaaagtttaaacaaaaatgtcaagggtgggcgcgccgacccagctttcttgtacaaagttggcattataagaaagcattgcttatcaatttgttgcaacgaacaggtcactatcagtcaaaataaaatcattatttgccatccagctgatatcccctatagtgagtcgtattacatggtcatagctgtttcctggcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtt

SEQ ID NO: 132 - protein, 269 aa, Artificial Sequence
  plasmid pENTR-DT.2 BaeIv.2 ccdB DT
MSHIQRETSRPRLNSNMDADLYGYKWARDNVGQSGATIYRLYGKPDAPELFLKHGKGSVANDVTDEMVRLNWLTEFMPLPTIKHFIRTPDDAWLLTTAIPGKTAFQVLEEYPDSGENIVDALAVFLRRLHSIPVCNCPFNSDRVFRLAQAQSRMNNGLVDASDFDDERNGWPVEQVWKEMHKLLPFSPDSVVTHGDFSLDNLIFDEGKLIGCIDVGRVGIADRYQDLAILWNCLGEFSPSLQKRLFQKYGIDNPDMNKLQFHLMLDEFF

SEQ ID NO: 133 - protein, 32 aa, Artificial Sequence
  attL2 recombination site
PAFLYKVGIIRKHCLSICCNEQVTISQNKIII

SEQ ID NO: 134 - protein, 32 aa, Artificial Sequence; identical to SEQ ID NO: 133
  attL2 recombination site

SEQ ID NO: 135 - protein, 6 aa, Artificial Sequence
  Tag sequence
CCRECC

SEQ ID NO: 136 - protein, 6 aa, Artificial Sequence
  Tag sequence
CCPGCC

SEQ ID NO: 137 - protein, 12 aa, Artificial Sequence
  Tag sequence
AGGCCPGCCGGG

SEQ ID NO: 138 - protein, 12 aa, Artificial Sequence
  Tag sequence
AGGCCPGCCGGG

SEQ ID NO: 139 - DNA, 36 bp, Artificial Sequence
  Sequence encoding Tag
gctggtggctgttgtcctggctgttgcggtggcggc

SEQ ID NO: 140 - DNA, 36 bp, Artificial Sequence
  Sequence encoding Tag
gccggcggctgttgtcctggctgttgcggtggcggc

SEQ ID NO: 141 - DNA, 36 bp, Artificial Sequence
  Sequence encoding Tag
gctggtggctgctgccctggctgttgcggtggcggc

SEQ ID NO: 142 - DNA, 36 bp, Artificial Sequence; identical to SEQ ID NO: 139
  Sequence encoding Tag

SEQ ID NO: 143 - DNA, 36 bp, Artificial Sequence
  Sequence encoding Tag
gctggtggctgttgtccaggctgttgcggtggcggc

SEQ ID NO: 144 - protein, 6 aa, Artificial Sequence
  Tag sequence
CCPGCC

SEQ ID NO: 145 - protein, 10 aa, Artificial Sequence
  Internally located Tag sequence
GGCCPGCCGG

SEQ ID NO: 146 - protein, 8 aa, Artificial Sequence
  attB1 recombination site
PAFLYKVV

SEQ ID NO: 147 - protein, 7 aa, Artificial Sequence
  attB1 recombination site
AFLYKVV

SEQ ID NO: 148 - protein, 6 aa, Artificial Sequence
  attB1 recombination site
FLYKVV

SEQ ID NO: 149 - protein, 5 aa, Artificial Sequence
  attB1 recombination site
LYKVV

SEQ ID NO: 150 - protein, 42 aa, Artificial Sequence
  attB1 recombination site
PAFLYKVVXXXXXXXXXXXXXXXXXXXXMDPETLVKVKDAED

SEQ ID NO: 151 - protein, 24 aa, Artificial Sequence
  attB1 recombination site
VVXXXXXXXXXXXXXXXXXXXXMD

SEQ ID NO: 152 - protein, 32 aa, Artificial Sequence
  attB1 recombination site
KVVXXXXXXXXXXXXXXXXXXXXMDPETLVKV

SEQ ID NO: 153 - protein, 26 aa, Artificial Sequence
  attB1 recombination site
VVXXXXXXXXXXXXXXXXXXXXMDPE

SEQ ID NO: 154 - protein, 265 aa, Artificial Sequence
  Polypeptide having beta-lactamase activity
MGHPETLVKVKDAEDQLGARVGYIELDLNSGKILESFRPEERFPMMSTFKVLLCGAVLSRDDAGQEQLGRRIHYSQNDLVEYSPVTEKHLTDGMTVRELCSAAITMSDNTAANLLLTTIGGPKELTAFLHNMGDHVTRLDHWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHW

SEQ ID NO: 155 - protein, 12 aa, Artificial Sequence
  Tag sequence
AGGCCPGCCGGG

SEQ ID NO: 156 - DNA, 15 bp, Artificial Sequence
  Core region of the wild type att sites
gcttttttatactaa

SEQ ID NO: 157 - DNA, 21 bp, Artificial Sequence
  Core sequence of each att site
caacttttttatacaaagttg

SEQ ID NO: 158 - DNA, 25 bp, Artificial Sequence
  attB1 sequence
agcctgcttttttgtacaaacttgt

SEQ ID NO: 159 - DNA, 233 bp, Artificial Sequence
  attP1 sequence
tacaggtcactaataccatctaagtagttgattcatagtgactggatatgttgtgttttacagtattatgtagtctgttttttatgcaaaatctaatttaatatattgatatttatatcattttacgtttctcgttcagcttttttgtacaaagttggcattataaaaaagcattgctcatcaatttgttgcaacgaacaggtcactatcagtcaaaataaaatcattatttg

SEQ ID NO: 160 - DNA, 100 bp, Artificial Sequence
  attL1 sequence
caaataatgattttattttgactgatagtgacctgttcgttgcaacaaattgataagcaatgcttttttataatgccaactttgtacaaaaaagcaggct

SEQ ID NO: 161 - DNA, 125 bp, Artificial Sequence
  attR1 sequence
acaagtttgtacaaaaaagctgaacgagaaacgtaaaatgatataaatatcaatatattaaattagattttgcataaaaaacagactacataatactgtaaaacacaacatatccagtcactatg

SEQ ID NO: 162 - DNA, 27 bp, Artificial Sequence
  attB0 sequence
agcctgcttttttatactaacttgagc

SEQ ID NO: 163 - DNA, 27 bp, Artificial Sequence
  attP0 sequence
gttcagcttttttatactaagttggca

SEQ ID NO: 164 - DNA, 27 bp, Artificial Sequence
  attL0 sequence
agcctgcttttttatactaagttggca

SEQ ID NO: 165 - DNA, 27 bp, Artificial Sequence
  attR0 sequence
gttcagcttttttatactaacttgagc

SEQ ID NO: 166 - DNA, 25 bp, Artificial Sequence
  attB1 sequence
agcctgcttttttgtacaaacttgt

SEQ ID NO: 167 - DNA, 27 bp, Artificial Sequence
  attP1 sequence
gttcagcttttttgtacaaagttggca

SEQ ID NO: 168 - DNA, 27 bp, Artificial Sequence
  attL1 sequence
agcctgcttttttgtacaaagttggca

SEQ ID NO: 169 - DNA, 25 bp, Artificial Sequence
  attR1 sequence
gttcagcttttttgtacaaacttgt

SEQ ID NO: 170 - DNA, 25 bp, Artificial Sequence
  attR2 sequence
acccagctttcttgtacaaagtggt

SEQ ID NO: 171 - DNA, 27 bp, Artificial Sequence
  attP2 sequence
gttcagctttcttgtacaaagttggca

SEQ ID NO: 172 - DNA, 27 bp, Artificial Sequence
  attL2 sequence
acccagctttcttgtacaaagttggca

SEQ ID NO: 173 - DNA, 25 bp, Artificial Sequence
  attR2 sequence
gttcagctttcttgtacaaagtggt

SEQ ID NO: 174 - DNA, 22 bp, Artificial Sequence
  attB5 sequence
caactttattatacaaagttgt

SEQ ID NO: 175 - DNA, 27 bp, Artificial Sequence
  attP5 sequence
gttcaactttattatacaaagttggca

SEQ ID NO: 176 - DNA, 24 bp, Artificial Sequence
  attL5 sequence
caactttattatacaaagttggca

SEQ ID NO: 177 - DNA, 25 bp, Artificial Sequence
  attR5 sequence
gttcaactttattatacaaagttgt

SEQ ID NO: 178 - DNA, 22 bp, Artificial Sequence
  attB11 sequence
caacttttctatacaaagttgt

SEQ ID NO: 179 - DNA, 27 bp, Artificial Sequence
  attP11 sequence
gttcaacttttctatacaaagttggca

SEQ ID NO: 180 - DNA, 24 bp, Artificial Sequence
  attL11 sequence
caacttttctatacaaagttggca

SEQ ID NO: 181 - DNA, 25 bp, Artificial Sequence
  attR11 sequence
gttcaacttttctatacaaagttgt

SEQ ID NO: 182 - DNA, 22 bp, Artificial Sequence
  attB17 sequence
caacttttgtatacaaagttgt

SEQ ID NO: 183 - DNA, 27 bp, Artificial Sequence
  attP17 sequence
gttcaacttttgtatacaaagttggca

SEQ ID NO: 184 - DNA, 24 bp, Artificial Sequence
  attL17 sequence
caacttttgtatacaaagttggca

SEQ ID NO: 185 - DNA, 25 bp, Artificial Sequence
  attR17 sequence
gttcaacttttgtatacaaagttgt

SEQ ID NO: 186 - DNA, 22 bp, Artificial Sequence
  attB19 sequence
caactttttcgtacaaagttgt

SEQ ID NO: 187 - DNA, 27 bp, Artificial Sequence
  attP19 sequence
gttcaactttttcgtacaaagttggca

SEQ ID NO: 188 - DNA, 24 bp, Artificial Sequence
  attL19 sequence
caactttttcgtacaaagttggca

SEQ ID NO: 189 - DNA, 25 bp, Artificial Sequence
  attR19 sequence
gttcaactttttcgtacaaagttgt

SEQ ID NO: 190 - DNA, 22 bp, Artificial Sequence
  attB20 sequence
caactttttggtacaaagttgt

SEQ ID NO: 191 - DNA, 27 bp, Artificial Sequence
  attP20 sequence
gttcaactttttggtacaaagttggca

SEQ ID NO: 192 - DNA, 24 bp, Artificial Sequence
  attL20 sequence
caactttttggtacaaagttggca

SEQ ID NO: 193 - DNA, 25 bp, Artificial Sequence
  attR20 sequence
gttcaactttttggtacaaagttgt

SEQ ID NO: 194 - DNA, 22 bp, Artificial Sequence
  attB21 sequence
caactttttaatacaaagttgt

SEQ ID NO: 195 - DNA, 27 bp, Artificial Sequence
  attP21 sequence
gttcaactttttaatacaaagttggca

SEQ ID NO: 196 - DNA, 24 bp, Artificial Sequence
  attL21 sequence
caactttttaatacaaagttggca

SEQ ID NO: 197 - DNA, 25 bp, Artificial Sequence
  attR21 sequence
gttcaactttttaatacaaagttgt

SEQ ID NO: 198 - DNA, 63 bp, Artificial Sequence
  Sense strand of the oligonucleotides forming the V5-2/FLS-1 polylinker
agctgagcgctgttaacgggaagcctatccctaaccctctcctcggtctcgattctacgcgta

SEQ ID NO: 199 - DNA, 63 bp, Artificial Sequence
  complementary strand of the oligonucleotides forming the V5-2/FLS-1 polylinker
ccggtacgcgtagaatcgagaccgaggagagggttagggataggcttcccgttaacagcgctc

SEQ ID NO: 200 - DNA, 24 bp, Artificial Sequence
  Oligonucleotide, ntermbla5
caccatggacccagaaacgctggt

SEQ ID NO: 201 - DNA, 20 bp, Artificial Sequence
  Oligonucleotide, ntermbla3
caatgcttaatcagtgaggc

SEQ ID NO: 202 - DNA, 20 bp, Artificial Sequence
  Oligonucleotide, ctermbla5
atggacccagaaacgctggt

SEQ ID NO: 203 - DNA, 20 bp, Artificial Sequence
  Oligonucleotide, ctbla3stop
ttaccaatgcttaatcagtg

SEQ ID NO: 204 - DNA, 53 bp, Artificial Sequence
  B1beta primer used to create pENTR/GeneBlazerNS
ggggacaagtttgtacaaaaaagcaggcaccatggacccagaaacgctggtga

SEQ ID NO: 205 - DNA, 52 bp, Artificial Sequence
  b2betaTAGA primer used to create pENTR/GeneBlazerNS
ggggaccactttgtacaagaaagctgtctaccaatgcttaatcagtgaggca

SEQ ID NO: 206 - DNA, 40 bp, Artificial Sequence
  SC1 primer used to amplify the Spectinomycin and ccdB genes
caccgacatttttgtttaaactttggtacctggatccttt

SEQ ID NO: 207 - DNA, 59 bp, Artificial Sequence
  SC2 primer used to amplify the Spectinomycin and ccdB genes
gacatttttgtttaaactttggtacctggatcctttaattatttgccgactaccttggt

SEQ ID NO: 208 - protein, 12 aa, Artificial Sequence
  Optimized LUMIO Binding Motif
AGGCCPGCCGGG

SEQ ID NO: 209 - DNA, 36 bp, Artificial Sequence; identical to SEQ ID NO: 139
  Encodes the optimized LUMIO Binding Motif

SEQ ID NO: 210 - DNA, 70 bp, Artificial Sequence
  Primer, For151tev1
ccatggtgctggtggctgttgtcctggctgttgcggtggcggcgaaaccctgtatattcagggaattatc

SEQ ID NO: 211 - DNA, 24 bp, Artificial Sequence
  Primer, 448 PCR.BLUNTT137
agactttatctgacagcagacgtg

SEQ ID NO: 212 - DNA, 77 bp, Artificial Sequence
  Primer, 5′42TOP
cgaagcttgaagctggtggctgttgtcctggctgttgcggtggcggcaccggtcatcatcaccatcaccatggttga

SEQ ID NO: 213 - DNA, 79 bp, Artificial Sequence
  Primer, 5′42BOT
ccggtcaaccatggtgatggtgatgatgaccggtgccgccaccgcaacagccaggacaacagccaccagcttcaagctt

SEQ ID NO: 214 - DNA, 28 bp, Artificial Sequence
  Primer, For Nde 42
cggaggtcatatgattatcacaagtttg

SEQ ID NO: 215 - DNA, 24 bp, Artificial Sequence
  Primer, Rev NdeI 42
gaaaatctcgccggatcctaactc

SEQ ID NO: 216 - DNA, 21 bp, Artificial Sequence
  T7 Forward Primer
taatacgactcactatagggg

SEQ ID NO: 217 - DNA, 28 bp, Artificial Sequence
  T7 Tag Rev Primer
cggtcggattaatacccatttgctgtcc

SEQ ID NO: 218 - DNA, 22 bp, Artificial Sequence
  Primer, DTOPO CAT-STOP rev
ctacgcccgccctgccactcat

SEQ ID NO: 219 - DNA, 23 bp, Artificial Sequence
  Primer, DTOPO CAT-NS rev
cgccccgccctgccactcatagt

SEQ ID NO: 220 - DNA, 20 bp, Artificial Sequence
  T7 Promoter Primer
taatacgactcactataggg

SEQ ID NO: 221 - DNA, 19 bp, Artificial Sequence
  TK polyA Reverse Primer
cttccgtgtttcagttagc

SEQ ID NO: 222 - DNA, 15 bp, Artificial Sequence
  Sequence of the N-terminus of a theoretical protein
atgggatctgataaa

SEQ ID NO: 223 - DNA, 18 bp, Artificial Sequence
  Proposed sequence forward PCR primer
accatgggatctgataaa

SEQ ID NO: 224 - DNA, 27 bp, Artificial Sequence
  Sequence of the C-terminus of a theoretical protein
aagtcggagcactcgacgacggtgtga

SEQ ID NO: 225 - DNA, 27 bp, Artificial Sequence
  Sequence of the C-terminus of a theoretical protein
aagtcggagcactcgacgacggtgtga

SEQ ID NO: 226 - DNA, 17 bp, Artificial Sequence
  Proposed Reverse PCR primer sequence
tgagctgctgccacaaa

SEQ ID NO: 227 - DNA, 33 bp, Artificial Sequence
  Sequence of the C-terminus of a theoretical protein
gcggttaagtcggagcactcgacgactgcatga

SEQ ID NO: 228 - DNA, 24 bp, Artificial Sequence
  Reverse Primer
tgcagtcgtcgagtgctccgactt

SEQ ID NO: 229 - DNA, 27 bp, Artificial Sequence
  Reverse primer with stop codon
tcatgcagtcgtcgagtgctccgactt

SEQ ID NO: 230 - DNA, 5380 bp, Artificial Sequence
  Sequence for pGeneBLAzer-TOPO
gacggatcgggagatctaatacgactcactatagggagacccaagctggctagcgtttaaacttaagcttggtaccgagctcggatccactagtccagtgtggtggaattgcccttaagggcaattcgcccttcaccatggacccagaaacgctggtgaaagtaaaagatgctgaagatcagttgggtgcccgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaagataaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 231 - DNA, 7519 bp, Artificial Sequence
  Sequence for pcDNA6.2/cGeneBLAzer-DEST
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagctgagcatcaacaagtttgtacaaaaaagctgaacgagaaacgtaaaatgatataaatatcaatatattaaattagattttgcataaaaaacagactacataatactgtaaaacacaacatatccagtcactatggcggccgcattaggcaccccaggctttacactttatgcttccggctcgtataatgtgtggattttgagttaggatccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaagatctggatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctcccttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggttgatgctgttatggacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 232 - DNA, 7519 bp, Artificial Sequence; identical to SEQ ID NO: 231
  Sequence for pcDNA6.2/nGeneBLAzer-DEST

SEQ ID NO: 233 - DNA, 5900 bp, Artificial Sequence
  Sequence for pcDNA6.2/cGeneBLAzer GW/D-TOPO
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagctgagcatcaacaagtttgtacaaaaaagcaggctccgcggccgcccccttcaccaagggtgggcgcgccgacccagctttcttgtacaaagtggttgatgctgttatggacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 234 - DNA, 5900 bp, Artificial Sequence; identical to SEQ ID NO: 233
  Sequence for pcDNA6.2/nGeneBLAzer GW/D-TOPO

SEQ ID NO: 235 - DNA, 4124 bp, Artificial Sequence
  Sequence for pENTR/GeneBLAzer
ctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgctagcatggatgttttcccagtcacgacgttgtaaaacgacggccagtcttaagctcgggccccaaataatgattttattttgactgatagtgacctgttcgttgcaacaaattgatgagcaatgcttttttataatgccaactttgtacaaaaaagcaggcaccatggacccagaaacgctggtgaaagtaaaagatgctgaagatcagttgggtgcacgagtgggttacatcgaactggatctcaacagcggtaagatccttgagagttttcgccccgctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgctagcatggatgttttcccagtcacgacgttgtaaaacgacggccagtcttaagctcgggccccaaataatgattttattttgactgatagtgacctgttcgttgcaacaaattgatgagcaatgcttttttataatgccaactttgtacaaaaaagcaggcaccatggacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtagacagctttcttgtacaaagttggcattataagaaagcattgcttatcaatttgttgcaacgaacaggtcactatcagtcaaaataaaatcattatttgccatccagctgatatcccctatagtgagtcgtattacatggtcatagctgtttcctggcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtt

SEQ ID NO: 236 - DNA, 6809 bp, Artificial Sequence; identical to SEQ ID NO: 87
  Sequence for pcDNA6.2/cFLASH-DEST

SEQ ID NO: 237 - DNA, 6809 bp, Artificial Sequence; identical to SEQ ID NO: 101
  Sequence for pcDNA6.2/nFLASH-DEST

SEQ ID NO: 238 - DNA, 5911 bp, Artificial Sequence
  Sequence for pcDNA6.2/cFLASH GW/TOPO
cgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagctgagcatcaacaagtttgtacaaaaaagcaggctccgcggccgcccccttcaccgacattttgtttaaactttggtacctggatcctttaaacgcgtggatccggcttactaaaagccagataacagtatgcgtatttgcgcgctgatttttgcggtataagaatatatactgatatgtatacccgaagtatgtcaaaaagaggtgtgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacagggactggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataattaaaggatccaggtaccaaagtttaaacaaaatgtcaagggtgggcgcgccgacccagctttcttgtacaaagtggttgatgctgttaacgggaagcctatccctaaccctctcctcggtctcgattctacgcgtaccggtgctggtggctgttgtcctggctgttgcggtggcggctagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcg

SEQ ID NO: 239 - DNA, 5932 bp, Artificial Sequence
  Sequence for pcDNA6.2/nFLASH GW/TOPO
cgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagctgcaccatggctggtggctgttgtcctggctgttgcggtggcggcaagctgggtaagcctatccctaaccctctcctcggtctcgattctacgagtgctgttatcacaagtttgtacaaaaaagcaggctccgcggccgcccccttcaccgacattttgtttaaactttggtacctggatcctttaaacgcgtggatccggcttactaaaagccagataacagtatgcgtatttgcgcgctgatttttgcggtataagaatatatactgatatgtatacccgaagtatgtcaaaaagaggtgtgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacagggactggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataattaaaggatccaggtaccaaagtttaaacaaaatgtcaagggtgggcgcgccgacccagctttcttgtacaaagtggtgataattaattaagataacaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcg

SEQ ID NO: 240 - DNA, 4608 bp, Artificial Sequence
  Sequence for D-T Entry ccdb spec
gtaaaacgacggccagtcttaagctcgggccccaaataatgattttattttgactgatagtgacctgttcgttgcaacaaattgatgagcaatgcttttttataatgccaactttgtacaaaaaagcaggctccgcggccgcccccttgacatttttgtttaaactttggtacctggatcctttaattatattccccagaacatcaggttaatggcgtttttgatgtcattttcgcggtggctgagatcagccacttcttccccgataacggagaccggcacactggccatatcggtggtcatcatgcgccagctttcatccccgatatgcaccaccgggtaaagttcacgggagactttatctgacagcagacgtgcactggccagggggatcaccatccgtcgcccgggcgtgtcaataatatcactctgtacatccacaaacagacgataacggctctctcttttataggtgtaaaccttaaactgcatttcaccagcccctgttctcgtcagcaaaagagccgttcatttcaataaaccgggcgacctcagccatcccttcctgattttccgctttccagcgttcggcacgcagacgacgggcttcattctgcatggttgtgcttaccagaccggagatattgacatcatatatgccttgagcaactgatagctgtcgctgtcaactgtcactgtaatacgctgcttcatagcatacctctttttgacatacttcgggtatacatatcagtatatattcttataccgcaaaaatcagcgcgcaaatacgcatactgttatctggcttttagtaagccggatccacgcgtttacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatgaaacgaattcgcccttcggagtactaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcctgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataattaaaggatccaggtaccaaagtttaaacaaaaatgtcaagggtgggcgcgccgacccagctttcttgtacaaagttggcattataagaaagcattgcttatcaatttgttgcaacgaacaggtcactatcagtcaaaataaaatcattatttgccatccagctgatatcccctatagtgagtcgtattacatggtcatagctgtttcctggcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgctagcatggatgttttcccagtcacgacgtt

SEQ ID NO: 241 - protein, 6 aa, Artificial Sequence
  Tag sequence
CCXXCC

SEQ ID NO: 242 - protein, 4 aa, Artificial Sequence
  Peptides which may be located at either the N-terminus, the C-terminus, or both termini of the tag
GGGG

SEQ ID NO: 243 - protein, 5 aa, Artificial Sequence
  Peptides which may be located at either the N-terminus, the C-terminus, or both termini of the tag
GGGGS

SEQ ID NO: 244 - protein, 4 aa, Artificial Sequence
  attB1 recombination site
YKVV

SEQ ID NO: 245 - DNA, 12 bp, Artificial Sequence
  Primer, TopoD-74
ggtgaagggggc

SEQ ID NO: 246 - DNA, 28 bp, Artificial Sequence
  Primer, Topo-75
cgcgcccacccttgacatagtacagttg

SEQ ID NO: 247 - DNA, 9 bp, Artificial Sequence
  Primer, TopoD-76
aagggtggg

SEQ ID NO: 248 - DNA, 31 bp, Artificial Sequence; identical to SEQ ID NO: 9
  Primer, TopoD-90

SEQ ID NO: 249 - DNA, 12 bp, Artificial Sequence
  Primer, TopoD-70
ctgtactatgtc

SEQ ID NO: 250 - DNA, 27 bp, Artificial Sequence
  Primer, DTOPO CAT for
caccatggagaaaaaaatcactggata

SEQ ID NO: 251 - DNA, 29 bp, Artificial Sequence
  Forward Primer used to generate the beta-lactamase gene
caccatggacccagaaacgctggtgaaag

SEQ ID NO: 252 - DNA, 30 bp, Artificial Sequence
  Reverse Primer used to generate the beta-lactamase gene
cgattacttaccaatgcttaatcagtgagg

SEQ ID NO: 253 - DNA, 18 bp, Artificial Sequence
  Forward Primer used to amplify the UbC promoter
gacggatcgggagatctg

SEQ ID NO: 254 - DNA, 20 bp, Artificial Sequence
  Reverse Primer used to amplify the UbC promoter
ggtaccaagcttcgtctaac

SEQ ID NO: 255 - DNA, 19 bp, Artificial Sequence
  Primer, CATcacc
caccatggagaaaaaaatc

SEQ ID NO: 256 - DNA, 23 bp, Artificial Sequence
  Primer, CATantiNS
ctacgccccgccctgccactcat

SEQ ID NO: 257 - DNA, 20 bp, Artificial Sequence
  Primer, CATantiS
cgccccgccctgccactcat